(19) United States
(12) Patent Application Publication
(10) Pub. No.: US 2025/0276074 A1
(43) Pub. Date: Sep. 4, 2025

(54) BRM AND BRG1 TARGETING ANTIBODY-DRUG CONJUGATES AND METHODS OF USE THEREOF

(71) Applicant: Prelude Therapeutics Incorporated, Wilmington, DE (US)

(72) Inventors: Andrew P. COMBS, Kenneth Square, PA (US); Andrew W. BUESKING, Wilmington, DE (US); Soham MAITY, Waltham, MA (US); Chun CHEN, Claymont, DE (US); Ryan HOLMES, Wilmington, DE (US); Sarah PAWLEY, Landenberg, PA (US); Jack David CARTER, Newark, DE (US); Koichi ITO, Philadelphia, PA (US); Max J. FOROUTAN, Bensalem, PA (US); Norman FULTANG, Philadelphia, PA (US); Peggy SCHERLE, Wayne, PA (US)

(21) Appl. No.: 19/067,422

(22) Filed: Feb. 28, 2025

Related U.S. Application Data
(60) Provisional application No. 63/743,815, filed on Jan. 10, 2025.
(60) Provisional application No. 63/710,947, filed on Oct. 23, 2024.
(60) Provisional application No. 63/687,997, filed on Aug. 28, 2024.
(60) Provisional application No. 63/560,527, filed on Mar. 1, 2024.

(51) Int. Cl. A61K 47/68; A61P 35/00

(52) U.S. Cl. CPC A61K 47/6803; A61K 47/6843; A61K 47/6849; A61K 47/6869; A61K 47/6889; A61P 35/00

(57) ABSTRACT

The present disclosure provides antibody drug conjugates and drug-linker compounds comprising one or more degrader compounds which comprise a target protein binding moiety and an E3 ubiquitin ligase binding moiety that binds a Von Hippel-Lindau E3 Ubiquitin Ligase or a Cereblon E3 Ubiquitin Ligase, and methods of use thereof.

### CROSS-REFERENCE(S) TO RELATED APPLICATION(S)

This application claims priority to U.S. Provisional Patent Application Nos. 63/560,527, filed Mar. 1, 2024, 63/687,997, filed Aug. 28, 2024, 63/710,947, filed Oct. 23, 2024, and 63/743,815, filed Jan. 10, 2025, each of which is incorporated herein by reference in their entirety.

### BACKGROUND OF THE INVENTION

The human SWItch/Sucrose Non-Fermentable (SWI/SNF) complexes are ATP-dependent chromatin remodelers. These large complexes play important roles in essential cellular processes, such as transcription, DNA repair and replication by regulating DNA accessibility.

Mutations in the genes encoding up to 20 canonical SWI/SNF subunits are observed in nearly 20% of all human cancers with the highest frequency of mutations observed in rhabdoid tumors, female cancers (including ovarian, uterine, cervical and endometrial), lung adenocarcinoma, gastric adenocarcinoma, melanoma, esophageal, and renal clear cell carcinoma.

SMARCA2 (BRM) and SMARCA4 (BRG1) are the subunits containing catalytic ATPase domains, and they are essential for the function of SWI/SNF in perturbation of histone-DNA contacts, thereby providing access points to transcription factors and cognate DNA elements that facilitate gene activation and repression.

SMARCA2 and SMARCA4 share a high degree of homology (up to 75%). SMARCA4 is frequently mutated in primary tumors (i.e., deleted or inactivated), particularly in lung cancer (12%), melanoma, liver cancer and pancreatic cancer. SMARCA2 is one of the top essential genes in SMARCA4-mutant (deleted) cancer cell lines. This is because SMARCA4 deleted cancer cells exclusively rely on SMARCA2 ATPase activity for their chromatin remodeling activity for cellular functions such as cell proliferation, survival and growth. Thus, targeting SMARCA2 may be a promising therapeutic approach in SMARCA4-deficient cancers (genetic synthetic lethality).

Previous studies have demonstrated the strong synthetic lethality using gene expression manipulation such as RNAi; downregulating SMARCA2 gene expression in SMARCA4 mutated cancer cells results in suppression of cancer cell proliferation. However, SMARCA2/4 bromodomain inhibitors (e.g. PFI-3) exhibit none to minor effects on cell proliferation inhibition [Vangamudi et al. Cancer Res 2015]. This phenotypic discrepancy between gene expression downregulation and a small molecule-based approach led us to investigating protein degradation bispecific molecules in SMARCA4 deficient cancers.

SMARCA2 is also reported to play roles in multiple myeloma expressing t(4;14) chromosomal translocation [Chooi et al. Cancer Res abstract 2018]. SMARCA2 interacts with NSD2 and regulates gene expression such as PRL3 and CCND1. SMARCA2 gene expression downregulation with shRNA reduces cell cycle S phase and suppresses cell proliferation of t(4;14) MM cells.

Therapeutic compounds that inhibit or degrade SMARCA2 and/or SMARCA4 have been reported. However, in some instances unwanted toxicities can occur. There remains a need for compounds that inhibit or degrade SMARCA2 and/or SMARCA4 with more specific targeting that reduces these effects. Antibody-drug conjugates described herein comprising an antibody that targets specific antigens and comprising a compound that leads to the degradation of SMARCA2 and/or SMARCA4 address this unmet need.

### BRIEF SUMMARY OF THE INVENTION

The present disclosure provides Antibody Drug Conjugate compounds, compositions thereof, methods of using said compounds and compositions thereof for the treatment of cancer, and Degrader-Linker compounds.

In one aspect, provided is an Antibody Drug Conjugate compound, or a pharmaceutically acceptable salt thereof, having the structure of:

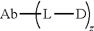

wherein,
Ab is an antibody or an antigen-binding fragment thereof,
L is a linker;
D is a degrader compound of Formula (I):

PTM-ULM (I)

wherein,
PTM is a moiety of Formula IA:

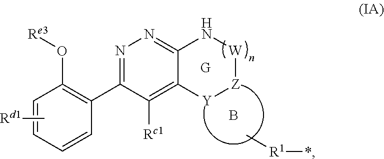

wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase; and
   subscript z is an integer ranging from 1 to 14.

In some aspects, provided is an Antibody Drug Conjugate compound, or a pharmaceutically acceptable salt thereof, of claim 1 having the structure of:

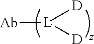

wherein,
Ab is an antibody or an antigen-binding fragment thereof,
each D is independently a degrader compound of Formula (I):

PTM-ULM (I),
wherein,
   PTM is a moiety of Formula IA:

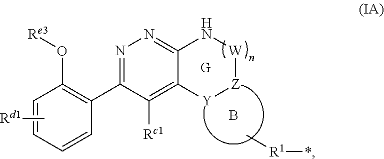

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃. 8 substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (III):

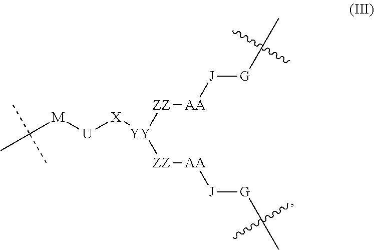

wherein,
   M is selected from the group consisting of:

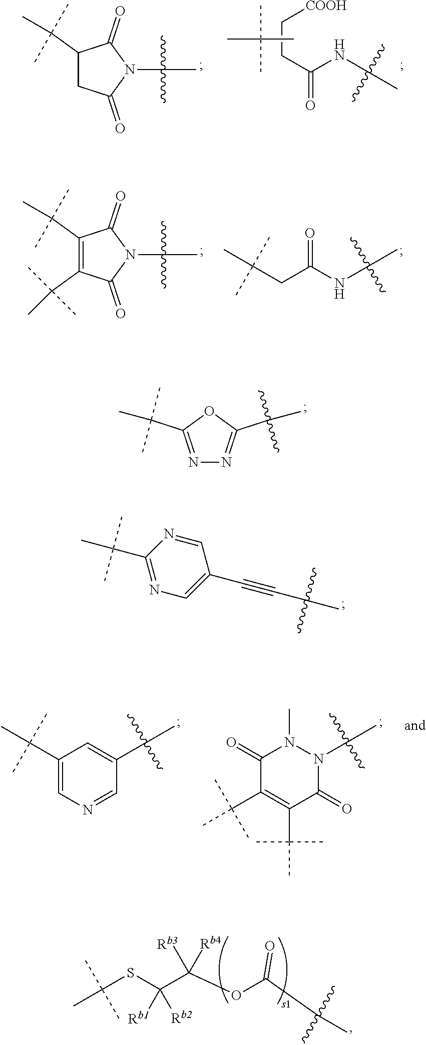

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1,
wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃,—CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—OC(O)—, wherein R^{x5} is hydrogen or C₁-C₆ alkyl and R^{x6} is hydrogen, C₁-C₆ alkyl, or

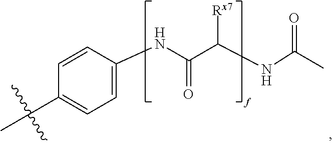

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of:

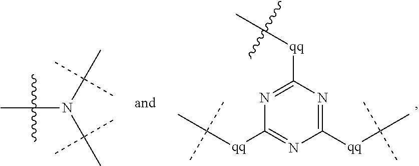

wherein
   each qq is independently —N(R^{y1})— or —O—, wherein R^{y1} is hydrogen or C₁-C₆ alkyl;
   the wavy line indicates the point of attachment to X, when present, or U, when X is absent, or M, when X and U are absent; and
   each dashed line indicates the point of attachment to ZZ when ZZ is present, or to AA when ZZ is absent, or to J when AA and ZZ are absent, or to G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of:

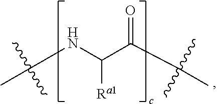

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

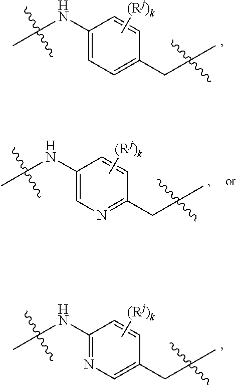

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
each G is independently absent,

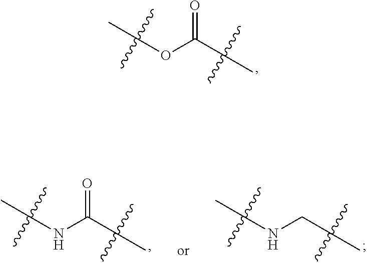

and
wherein the wavy line to M of Formula (III) indicates the point of covalent attachment to Ab and the wavy line to each G of Formula (III) indicates the point of covalent attachment to D,
wherein subscript z is an integer ranging from 1 to 14.

In some aspects, provided is a pharmaceutical composition comprising an Antibody Drug Conjugate compound described herein and at least one pharmaceutically acceptable excipient.

In some aspects, provided is a method of treating cancer in a subject in need thereof comprising administering to the subject an effective amount of an Antibody Drug Conjugate compound described herein, or a pharmaceutically acceptable salt thereof, or a pharmaceutical composition described herein.

In some aspects, provided is a Degrader-Linker compound, or a pharmaceutically acceptable salt or solvate thereof, having the structure of:

L′-D

wherein,
L′ is a linker precursor; and
D is a degrader compound of Formula (I):

PTM-ULM (I),
wherein,
   PTM is a moiety of Formula IA:

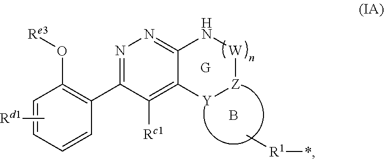

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z; and
   ULM is a small molecule E3 Ubiquitin Ligase binding moiety that binds a Von Hippel-Lindau E3 Ubiquitin Ligase or a Cereblon E3 Ubiquitin Ligase.

In some aspects, provided is a Degrader-Linker compound, or a pharmaceutically acceptable salt thereof, represented by the structure of:

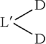

wherein,
each D is independently a degrader compound of Formula (I):

PTM-ULM (I),
wherein,
   PTM is a moiety of Formula IA:

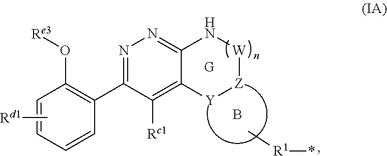

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L′ is a linker precursor of Formula (iii)

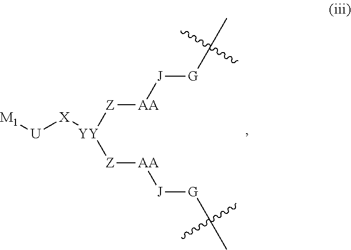

wherein
   M′ is selected from the group consisting of:

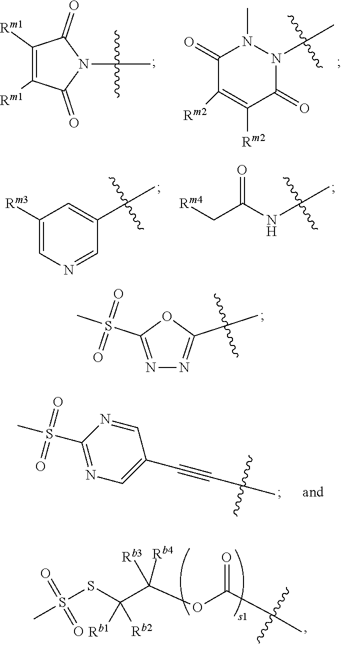

wherein
   each R^{m1} and R^{m2} is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b}a and R^{b4} are each independently hydrogen or C₁-C₆ alkyl, subscript s1 is 0 or 1;
   wherein the wavy line of M′ indicates the point of covalent attachment to the remainder of the structure of L′ and the wavy line of Formula (iii) indicates the point of covalent attachment to the degrader compound (D); and
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R², wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—OC(O)—, wherein R^{x5} is hydrogen or C₁-C₆ alkyl and R^{x6} is hydrogen, C₁-C₆ alkyl, or

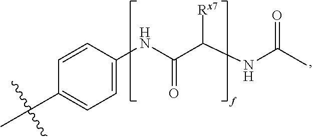

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of:

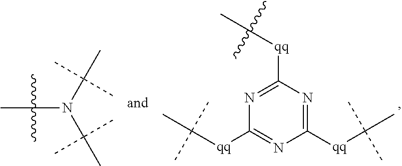

wherein
   each qq is independently —N(R^{y1})— or —O—, wherein R^{y1} is hydrogen or C₁-C₆ alkyl;
   the wavy line indicates the point of attachment to X, when present, or U, when X is absent, or M′, when X and U are absent; and
   each dashed line indicates the point of attachment to ZZ when ZZ is present, or AA when ZZ is absent, or J when AA and ZZ are absent, or G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of:

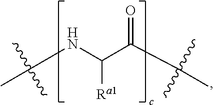

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

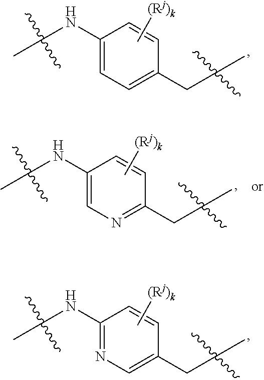

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
each G is independently absent,

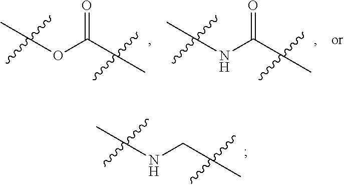

and
wherein the wavy line to each G of Formula (iii) indicates the point of covalent attachment to D.

### BRIEF DESCRIPTION OF THE DRAWINGS

FIGS. 1A and 1B show evaluation of anti-tumor activity (e.g., tumor growth inhibition) of Compound 1 and Compound 2 against PSMA+ prostate cancer cell line derived xenograft tumors (LNCaP) in CB17 SCID male mice and associated percent change in body weight, respectively.

FIGS. 2A-2C show analysis of SMARCA2 and SMARCA4 expression by Western blot.

FIGS. 3A-3B show evaluation of anti-tumor activity (e.g., tumor growth inhibition) of Compound 1 (squares), Compound 2 (triangles), and a vehicle (circles) against PSMA− prostate cancer cell line derived xenograft tumors (PC3) in BALC/c nude mice and associated percent change in body weight, respectively.

FIG. 4 shows anti-tumor activity (e.g., tumor growth inhibition) of Compound 3 and a comparator compound against PSMA+ prostate cancer cell line derived xenograft tumors (LNCaP) in CB17 SCID male mice.

FIG. 5A shows a ¹H-NMR (400 MHz, DMSO-d₆) spectrum of Compound 2.63.

FIG. 5B shows a ¹H-NMR (400 MHz, DMSO-d₆) spectrum of Compound 2.65.

FIG. 5C shows a ¹H-NMR (400 MHz, DMSO-d₆) spectrum of Compound 2.69.

### DETAILED DESCRIPTION OF THE INVENTION

Provided herein are antibody-drug conjugates that can target SMARCA2 and/or SMARCA4 and may be used in treatment of conditions such as cancer and autoimmune diseases.

### Definitions

The following description is presented to enable a person of ordinary skill in the art to make and use the various embodiments. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Reference to “about” a value or parameter herein includes (and describes) embodiments that are directed to that value or parameter per se. For example, description referring to “about X” includes description of “X”. In some embodiments, the term “about” when used in association with a measurement, or used to modify a value, a unit, a constant, or a range of values, refers to variations of +/−2%.

Reference to “between” two values or parameters herein includes (and describes) embodiments that include those two values or parameters per se. For example, description referring to “between x and y” includes description of “x” and “y” per se.

It is understood that aspects and variations described herein also include “consisting of” and/or “consisting essentially of” aspects and variations.

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these recited ranges are included in the range.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain aspects, one or more of the present conjugates described herein, are co-administered in combination with at least one additional bioactive agent, especially including an anticancer agent. In certain aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium. In some embodiments, “compound” refers to a degrader compound described herein, including tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in some embodiments, to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

The term “conjugate”, as used herein, unless otherwise indicated, refers to any specific antibody-drug conjugate disclosed herein, as well as pharmaceutically acceptable salts and derivatives thereof. In some instances, the term “conjugate” includes compositions and pharmaceutically acceptable compositions of antibody-drug conjugates described herein.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome.

However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

As used herein, “Von Hippel-Lindau (VHL) E3 Ubiquitin Ligase” and “Cereblon (CRBN) E3 Ubiquitin Ligase” refer to the substrate recognition subunit of Cullin RING E3 ubiquitin ligase complexes. Both VHL and CRBN are popular E3 ligases recruited by bifunctional Proteolysis-targeting chimeras (PROTACs) to induce ubiquitination and subsequent proteasomal degradation of a target protein (Girardini, M. et al., Bioorg Med Chem. 2019, 27(12): 2466-2479).

As used herein, the term “alkyl”, by itself or as part of another substituent, means, unless otherwise stated, a straight or branched chain hydrocarbon radical having up to twelve carbon atoms. In some embodiments, the number of carbon atoms is designated (i.e., C₁-C₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is a C₁-C₆ alkyl; in some embodiments, it is a C₁-C₄ alkyl.

When a range of carbon atoms is used herein, for example, C₁-C₆, all ranges, as well as individual numbers of carbon atoms are encompassed. For example, “C₁-C₃” includes C₁-C₃, C₁. C₂, C₂-C₃, C₁, C₂, and C₃.

The term “optionally substituted”, as used in combination with a substituent defined herein, means that the substituent may, but is not required to, have one or more hydrogens replaced with one or more suitable functional groups or other substituents as provided herein.

For example, a substituent may be optionally substituted with one or more of: —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C⁸alkyl), —OH, —NH₂, —SH, —SO₂C₁-C₈alkyl, SO(NH)C₁-C₅alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂. In some embodiments, each of the above optional substituents are themselves optionally substituted by one or two groups.

The term “optionally substituted —CH₂—,” refers to “—CH₂—” or “substituted —CH₂—.” A substituted —CH₂— may also be referred to as —CH(substituent)- or —C(substituent)(substituent)-, wherein each substituent is independently selected from the optional substituents described herein.

The term “cycloalkyl” as used herein refers to a 3-12 membered cyclic alkyl group, and includes bridged and spirocycles (e.g., adamantine). Cycloalkyl groups may be fully saturated or partially unsaturated. The term “cycloalkyl” also includes multiple condensed ring systems (e.g., ring systems comprising 2, 3 or 4 rings) wherein a single cycloalkyl ring (as defined above) can be condensed with one or more groups selected from heterocycles, carbocycles, aryls, or heteroaryls to form the multiple condensed ring system. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a cycloalkyl) can be at any position of the cycloalkylic ring. Examples of cycloalkyl groups include cyclopropyl, cyclobutyl, cyclopentyl, cycloheptyl, cyclohexyl, cycloheptyl, cyclooctyl, indenyl, bicyclo[2.2.1]heptanyl, bicyclo[3.1.1]heptanyl, bicyclo[4.1.0]heptanyl, spiro[3.3]heptanyl, and spiro[3.4]octanyl. In some embodiments, the cycloalkyl group is a 3-7 membered cycloalkyl.

The term “alkenyl” as used herein refers to C₂-C₁₂ alkyl group that contains at least one carbon-carbon double bond. In some embodiments, the alkenyl group is optionally substituted.

In some embodiments, the alkenyl group is a C₂-C₆ alkenyl.

The term “akynyl” as used herein refers to C₂-C₁₂ alkyl group that contains at least one carbon-carbon triple bond. In some embodiments, the alkenyl group is optionally substituted. In some embodiments, the alkynyl group is a C₂-C₆ alkynyl.

The terms “alkoxy,” “alkylamino” and “alkylthio”, are used in their conventional sense, and refer to those alkyl groups attached to the remainder of the molecule via an oxygen atom (“oxy”), an amino group (“amino”) or thio group. The term “alkylamino” includes mono-di-alkylamino groups, the alkyl portions can be the same or different.

The terms “halo” or “halogen”, by itself or as part of another substituent, means a fluorine, chlorine, bromine, or iodine atom.

The term “heteroalkyl” refers to an alkyl group in which one or more carbon atom has been replaced by a heteroatom selected from S, O, P and N. Exemplary heteroalkyls include alkyl ethers, secondary and tertiary alkyl amines, alkyl amides, alkyl sulfides, and the like. The group may be a terminal group or a bridging group. As used herein reference to the normal chain when used in the context of a bridging group refers to the direct chain of atoms linking the two terminal positions of the bridging group.

The term “aryl” as used herein refers to a single, all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the aromatic ring. Non-limiting examples of aryl groups include, but are not limited to, phenyl, indenyl, naphthyl, 1, 2, 3, 4-tetrahydronaphth-yl, and the like.

The term “Ph” as used herein refers to phenyl.

The term “heteroaryl” as used herein refers to a single aromatic ring that has at least one atom other than carbon in the ring, wherein the atoms are selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example a naphthyridinyl such as 1,8-naphthyridinyl), heterocycles, (to form for example a 1, 2, 3, 4-tetra-hydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 12 or about 5 to 10 members within the heteroaryl ring. Multiple condensed ring systems may be optionally substituted with one or more (e.g., 1, 2, 3 or 4) oxo groups on the carbocycle or heterocycle portions of the condensed ring. The rings of a multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the heteroaryl ring. It is also to be understood that the point of attachment for a heteroaryl or heteroaryl multiple condensed ring system can be at any suitable atom of the heteroaryl ring including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The term “heterocyclyl” or “heterocycle” as used herein refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. Accordingly, a heterocycle (a single saturated or single partially unsaturated ring or multiple condensed ring system) has about 3-20 atoms including about 1-6 heteroatoms within the heterocycle ring system. It is also to be understood that the point of attachment of a multiple condensed ring system (as defined above for a heterocylyl) can be at any position of the heterocyclic ring. It is also to be understood that the point of attachment for a heterocycle or heterocycle multiple condensed ring system can be at any suitable atom of the heterocyclic ring including a carbon atom and a heteroatom (e.g., a nitrogen). In one embodiment the term heterocycle includes a C₂₋₂₀ heterocycle. In one embodiment the term heterocycle includes a C₂₋₇ heterocycle. In one embodiment the term heterocycle includes a C₂₋₅ heterocycle. In one embodiment the term heterocycle includes a C₂₋₄ heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydro-quinolyl, benzoxazinyl, dihydrooxazolyl, chromanyl, 1,2-dihydropyridinyl, 2,3-dihydrobenzo-furanyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrahydrothiophene, quinuclidine, tropane, 2-azaspiro[3.3]-heptane, (1R,5S)-3-azabicyclo[3.2.1]octane, (1s,4s)-2-azabicyclo[2.2.2]octane, (1R,4R)-2-oxa-5-azabicyclo[2.2.2]octane and pyrrolidin-2-one. In one embodiment the term “heterocycle” refers to a monocyclic, saturated or partially unsaturated, 3-8 membered ring having at least one heteroatom. For example, the term includes a monocyclic, saturated or partially unsaturated, 4, 5, 6, or 7 membered ring having at least one heteroatom. Non-limiting examples of heterocycle include aziridine, azetidine, pyrrolidine, piperidine, piperidine, piperazine, oxirane, morpholine, and thiomorpholine. The term “9- or 10-membered heterobicycle” as used herein refers to a partially unsaturated or aromatic fused bicyclic ring system having at least one heteroatom. For example, the term 9- or 10-membered heterobicycle includes a bicyclic ring system having a benzo ring fused to a 5-membered or 6-membered saturated, partially unsaturated, or aromatic ring that contains one or more heteroatoms.

As used herein, the term “heteroatom” is meant to include oxygen (O), nitrogen (N), sulfur (S) and silicon (Si). The nitrogen and sulfur can be in an oxidized form when feasible.

A “branching unit” as used herein refers to a chemical moiety that comprises at least three attachment sites (e.g., at least trifunctional). For example, an at least tri-substituted carbocycle, an at least tri-substituted heterocycle, a tertiary carbon atom, and a tertiary nitrogen atom can serve as a branching unit. In some embodiments, the branching unit comprises at least three functional groups independently selected from an amine, a carboxylate, a thiocarboxylate, hydroxyl, thiol, carbamate, thiocarbamate, sulfonate, sulfonamide, phosphonate, and phosphinate. The branching unit is typically present when more than one degrader compounds, as described herein, is to be conjugated to a linker or a linker precursor, as described herein. However, a branching unit can also be used to conjugate only one degrader compound to a linker or only one degrader to a linker precursor, as described herein. In some embodiments, a branching unit comprises three attachment sites.

As used herein, the term “stereoisomers” refers to compounds which have identical chemical constitution but differ with regard to the arrangement of the atoms or groups in space, e.g., enantiomers, diastereomers, tautomers.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition, conjugate, or component which, when used within the context of its intended use, effects an intended result. The term effective subsumes all other effective amount or effective concentration terms, which are otherwise described or used in the present application.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound or a conjugate of the disclosure that is pharmaceutically acceptable and that possesses the desired pharmacological activity of the parent compound or conjugate. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound or conjugate either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound or conjugate contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder. As used herein, including the appended claims, the singular forms of words such as “a,” “an,” and “the,” include their corresponding plural references unless the context clearly dictates otherwise.

An “antibody-drug conjugate” or “ADC” refers to an antibody conjugated to a drug moiety, such as a cytotoxic agent. Typically, an ADC binds to a target antigen on a cell surface, followed by internalization of the ADC into the cell and subsequent release of the drug into the cell. In some embodiments, the target antigen is PSMA, CD33, HER2, Trop-2, HER3, B7H3, B7H4, CEACAM5, MET, NECTIN4, CALR, or CD123.

The term “drug loading”, “drug-antibody ratio” or “DAR” used herein is the average number of drug moieties conjugated to each individual antibody.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures. Substituents such as carbohydrate groups are generally not specified, but may be present nonetheless.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

In antibodies or other proteins described herein, reference to amino acid residues corresponding to those specified by sequence identification number (i.e., SEQ ID NO) includes post-translational modifications of such residues.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The term “antibody” denotes immunoglobulin proteins produced by the body in response to the presence of an antigen and that bind to the antigen, as well as antigen-binding fragments and engineered variants thereof. Hence, the term “antibody” includes, for example, full length antibodies as well as antigen-binding antibody fragments, such as a F(ab′)2, a Fv fragment, a diabody, a single-chain antibody, an scFv fragment, or an scFv-Fc. Genetically, engineered antibodies and antibody fragments such as chimeric antibodies, humanized antibodies, fully human antibodies, single-chain Fv fragments, single-chain antibodies, diabodies, minibodies, linear antibodies, multivalent or multi-specific (e.g., bispecific) hybrid antibodies, and the like, are also included. The antibodies provided herein may be “half antibodies”, comprising a single light chain and a single heavy chain.

The term antibody or antigen-binding fragment thereof includes a “conjugated” antibody or antigen-binding fragment thereof or an “antibody-drug conjugate (ADC)” in which an antibody or antigen-binding fragment thereof is covalently or non-covalently bound to a pharmaceutical agent, e.g., to a drug moiety, such as a cytotoxic agent.

The term “genetically engineered antibodies” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, e.g., complement binding and other effector functions. Typically, changes in the variable region are made to improve antigen-binding characteristics, improve variable region stability, and/or reduce the risk of immunogenicity.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “human” antibody or antigen-binding fragment thereof means an antibody or antigen-binding fragment thereof having an amino acid sequence derived from a human immunoglobulin gene locus, where such antibody or antigen-binding fragment is made using techniques known in the art. This definition of a human antibody or antigen-binding fragment thereof includes intact or full-length antibodies and fragments thereof.

An “antigen-binding site of an antibody” is that portion of an antibody that is sufficient to bind to its antigen. The minimum such region is typically a variable domain or a genetically engineered variant thereof. Single domain binding sites can be generated from camelid antibodies (see Muyldermans and Lauwereys, Mol. Recog. 12: 131-140, 1999; Nguyen et al., EMBO J. 19:921-930, 2000) or from VH domains of other species to produce single-domain antibodies (“dAbs,” see Ward et al., Nature 341: 544-546, 1989; U.S. Pat. No. 6,248,516 to Winter et al). Commonly, an antigen-binding site of an antibody comprises both a heavy chain variable (V_{H}) domain and a light chain variable (V_{L}) domain that bind to a common epitope. Within the context of the present disclosure, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al., FEBS Letters 450:23-26, 1999), a cytotoxic agent, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, triabodies, tetrabodies, minibodies, nanobodies, single domain VNARs, Fab-scFv fusions, bispecific (scFv)4-IgG, and bispecific (scFv)2-Fab. (See, e.g., Hu et al., Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

The term “immunoglobulin” refers to a protein consisting of one or more polypeptides substantially encoded by immunoglobulin gene(s). One form of immunoglobulin constitutes the basic structural unit of native (i.e., natural or parental) antibodies in vertebrates. This form is a tetramer and consists of two identical pairs of immunoglobulin chains, each pair having one light chain and one heavy chain. In each pair, the light and heavy chain variable regions (V_{L} and V_{H}) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that consists of constant region protein domains (CH1, hinge, CH2, and CH3; IgG3 also contains a CH4 domain) that are essentially invariant for a given subclass in a species.

DNA sequences encoding human and non-human immunoglobulin chains are known in the art. (See, e.g., Ellison et al, DNA 1: 11-18, 1981; Ellison et al, Nucleic Acids Res. 10:4071-4079, 1982; Kenten et al., Proc. Natl. Acad. Set USA 79:6661-6665, 1982; Seno et al., Nucl. Acids Res. 11:719-726, 1983; Riechmann et al., Nature 332:323-327, 1988; Amster et al., Nucl. Acids Res. 8:2055-2065, 1980; Rusconi and Kohler, Nature 314:330-334, 1985; Boss et al., Nucl. Acids Res. 12:3791-3806, 1984; Bothwell et al., Nature 298:380-382, 1982; van der Loo et al., Immunogenetics 42:333-341, 1995; Karlin et al., J. Mol. Evol. 22: 195-208, 1985; Kindsvogel et al., DNA 1:335-343, 1982; Breiner et al., Gene 18: 165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31: 169-217, 1994.

Full-length immunoglobulin “light chains” (about 25 kDa or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 kDa or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“V_{L} domain”) or “heavy chain variable domain” (“VH domain”), respectively) consists of a “framework” region and three “complementarity determining regions” or “CDRs.” The framework regions align the CDRs for specific binding to an epitope of an antigen. Thus, the term “CDR” refers to the amino acid residues of an antibody that are primarily responsible for antigen binding. From amino-terminus to carboxyl-terminus, both V_{L} and VH domains comprise the following framework (FR) and CDR regions: FR1, CDR1, FR2, CDR2, FR3, CDR3, FR4.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a V_{L} domain are also referred to herein, respectively, as CDR-L1, CDR-L2 and CDR-L3. CDRs 1, 2 and 3 of a V_{H} domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

Numbering of the heavy chain constant region is via the EU index as set forth in Kabat (Kabat, Sequences of Proteins of Immunological Interest, National Institutes of Health, Bethesda, MD, 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

A “humanized antibody” is an antibody comprising one or both of a humanized VH domain and a humanized VL domain. Immunoglobulin constant region(s) need not be present, but if they are, they are entirely or substantially from human immunoglobulin constant regions.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence.

Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

Although humanized antibodies often incorporate all six CDRs (preferably as defined by Kabat or IMGT®) from a mouse antibody, they can also be made with fewer than all six CDRs (e.g., at least 3, 4, or 5) CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### I. Antibody Drug Conjugates (ADC)

Provided herein are Antibody Drug Conjugate compounds, or pharmaceutically acceptable salts thereof, having the structure of:

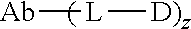

wherein, Ab is an antibody or an antigen-binding fragment thereof; L is a linker; D is a degrader compound; and subscript z is an integer ranging from 1 to 14, wherein L is covalently bound to Ab and D.

In some embodiments, subscript z represents the number of drug linker moieties conjugated to an antibody of an antibody-drug conjugate. In some embodiments, subscript z is an integer ranging from 1 to 8. In some embodiments, subscript z is an integer ranging from 1 to 4. In some embodiments, subscript z is 2. In some embodiments, subscript z is 4. In some embodiments, subscript z is 8. In some embodiments, there are 1, 2, 3, 4, 5, 6, 7, 8, 9, 10, 11, 12, 13, or 14 drug linker moieties conjugated to an antibody of an antibody-drug conjugate.

In some embodiments, descriptions herein of antibody-drug conjugates or compositions thereof relate to a number of antibody-drug conjugates that substantially differ only in the number of drug linker moieties conjugated to the antibody. In those embodiments, subscript z is a number that represents the average number of drug linker moieties conjugated to the antibody. In those embodiments, subscript z is a number ranging from about 1 to about 14, from about 1 to about 8, about 1 to about 4, about 2 to about 8, about 2 to about 4, or about 4 to about 8. In some of those embodiments, subscript z is about 8. In some of those embodiments, subscript z is about 2. In some of those embodiments, subscript z is about 4. In some embodiments, subscript z represents the average drug loading of the number of antibody-drug conjugates. In some embodiments, subscript z represents the drug loading of the predominate antibody-drug conjugate in the composition comprising a number of antibody-drug conjugates.

In some embodiments, reduced interchain disulfides will be the site of conjugation of a drug linker compound to an antibody. In some embodiments, 1 to 8 drug linker compounds will be conjugated to an antibody via reduced interchain disulfides. In some embodiments, 1 to 4 drug linker compounds will be conjugated to an antibody via reduced interchain disulfides. In some embodiments, an introduced cysteine will be the site of conjugation of a drug linker compound to an antibody. In some embodiments, 1 to 8 drug linker compounds will be conjugated to an antibody via an introduced cysteine. In some embodiments, 1 to 4 drug linker compounds will be conjugated to an antibody via an introduced cysteine. In some embodiments, reduced interchain disulfides as well as one or more introduced cysteines will be the site of conjugation of drug linker compounds to an antibody. In some embodiments, 1 to 8 drug linker compounds will be conjugated to an antibody via reduced interchain disulfides as well as one or more introduced cysteines. In some embodiments, 1 to 4 drug linker compounds will be conjugated to an antibody via reduced interchain disulfides as well as one or more introduced cysteines.

In some embodiments described herein, the sulfur atom of a side chain of the cysteine of an antibody is represented as “S”. For example, a sulfur atom of the cysteine of the antibody of the Antibody Drug Conjugate compounds shown in Table 1 is denoted as “S”.

### Antibodies, Antigen-Binding Fragments, and Antigens

A conventional antibody, also known as an immunoglobulin (Ig), is a large hetero-tetrameric protein which is composed of two heavy chains and two light chains. The heavy chain of a conventional IgG consists of a heavy chain variable (VH) domain and three constant domains (CH1, CH2 and CH3), wherein the CH1 and CH₂ are joined together by a hinge region, whereas the light chain consists of a light chain variable (VL) domain and a constant domain (CL). The fragment antigen-binding (Fab) is the antibody region that binds to antigens. The Fab is composed of one constant and one variable domain of each of the heavy and the light chain (i.e., VH-CH1 paired with VL-CL). Together, these domains shape the paratope and the antigen-binding site at the amino terminal end of the antibody Fab. The Fv region is similar to the Fab, but only contains one variable domain of each of the heavy and the light chain. The Fc (fragment, crystallizable) region is composed of two heavy chain fragments that contribute two (i.e., CH2 and CH3) or three constant domains, depending on the class of the antibody, and plays a role in modulating immune cell activity. Finally, the Fd region is the initial 220 residues of the amino terminal side of the heavy chain, which often includes the heavy chain variable (VH) domain and CH1 domain. The antibodies suitable for use in the ADCs disclosed herein can be full-length antibodies or antigen-binding fragments thereof.

In some aspects, provided herein is an ADC comprising an antibody or antigen-binding fragment thereof. A number of suitable antibodies and antigen-binding fragments can be used in the compositions and methods of the present invention. Antibodies suitable for use in the present invention are useful for a number of applications, including in vitro or in vivo diagnosis, in vivo imaging, and therapy for diseases and conditions associated with various target antigens. Five human antibody classes (IgG, IgA, IgM, IgD and IgE), as well as various subclasses (e.g., IgG1, IgG2, IgG3, IgG4, IgA1 and IgA2) within these classes, are recognized on the basis of structural differences, such as the number of immunoglobulin units in a single antibody molecule, the disulfide bridge structure of the individual units, and differences in chain length and sequence. The class and subclass of an antibody is referred to as the antibody's isotype.

The antibody suitable for use in the ADCs of the present disclosure can be a full-length antibody or an antigen-binding antibody fragment thereof. In some embodiments, the antibody or antigen-binding fragment contains at least one inter-chain disulfide bond. In some embodiments, the antibody is a full-length antibody. In some embodiments, the antibody is a full-length antibody selected from the group consisting of IgA, IgD, IgE, IgG, and IgM. In some embodiments, the antibody is selected from the group consisting of IgG, IgA, IgM, IgD and IgE antibody. In some embodiments, the antibody is an IgG antibody. In some embodiments, the antibody is of an isotype selected from the group consisting of IgG1, IgG2, IgG3, and IgG4 antibody. In some embodiments, the antibody is an IgA antibody. In some embodiments, the antibody is an IgA1 antibody or IgA2 antibody. In some embodiments, the light chain is a human kappa light chain. In some embodiments, the light chain is a human kappa lambda chain. In some embodiments, the antibody is monoclonal. In some embodiments, the antibody is polyclonal.

The antibodies suitable for use in the ADCs of the present disclosure can be post-translationally or chemically modified in ways that alter their activity, stability, half-life, secretion, immunogenicity, and/or and function. Antibody glycosylation is a major post-translational modification that influences antibody function. Particular asparagine residues in the antibody Fc region are modified by N-glycans, but residues in the Fab region can also be glycosylated. These residues are often considered and manipulated when engineering new antibody therapeutics to help tune the host immunological response and antibody antigenicity, respectively. Antibody glycosylation may include fucosylation, galactosylation, bisection and sialylation modifications. In IgG antibodies, heavy chain residue asparagine 297 (N297) according to Kabat numbering is glycosylated. In other antibody isotypes, additional residues in the heavy chain are glycosylated, wherein IgM (5), IgD (3), IgE (7), IgA1 (2) and IgA2 (5) each have the indicated number of N-linked glycosylation sites in their heavy chains, and wherein the glycans are more complex. IgD and IgA1 also possess multiple O-linked glycosylation sites. In some embodiments, the antibody or antigen-binding fragment glycosylation is N-linked. In some embodiments, the antibody or antigen-binding fragment glycosylation is O-linked. In some embodiments, the antibody or antigen-binding fragment glycosylation is biantennary. In some embodiments, the antibody or antigen-binding fragment glycosylation is triantennary or tetraantennary. In some embodiments, the antibody or antigen-binding fragment thereof is fucosylated. In some embodiments, the antibody or antigen-binding fragment thereof comprises a bisecting GlcNAc moiety. In some embodiments, the antibody or antigen-binding fragment thereof is monogalactosylated or digalactosylated. In some embodiments, the antibody or antigen-binding fragment thereof is sialyated. In some embodiments, the antibody or antigen-binding fragment thereof is modified such that sites of glycosylation within the antibody can no longer be glycosylated. In some embodiments, the antibody or antigen-binding fragment thereof comprises an amino acid substitution at the site of glycosylation. In some embodiments, the antibody or antigen-binding fragment thereof is afucosylated. In some embodiments, the antibody or antigen-binding fragment thereof is agalactosylated. In some embodiments, the antibody is asialylated.

Typically, the antibodies suitable for use in the ADCs of the present disclosure are human, rodent (e.g., mouse and rat), donkey, sheep, rabbit, goat, guinea pig, camelid, horse, or chicken. The antibody can be, for example, a murine, a chimeric, humanized, or fully human antibody produced by techniques well-known to one of skill in the art. In some embodiments, the antibody is chimeric, human or humanized. In some embodiments, the antibody is chimeric. In some embodiments, the antibody is human. In some embodiments, the antibody is murine. In some embodiments, the antibody is humanized.

Recombinant antibodies, such as chimeric and humanized monoclonal antibodies, comprising both human and non-human portions which can be made using standard recombinant DNA techniques, may be used as the antibodies in the ADCs of the present disclosure. A chimeric antibody is a molecule in which different portions are derived from different animal species, such as those having a variable region derived from a murine monoclonal and human immunoglobulin constant regions. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art. As used herein, “human” antibodies include antibodies having the amino acid sequence of a human immunoglobulin and include antibodies isolated from human immunoglobulin libraries, from human B cells, or from animals transgenic for one or more human immunoglobulin, as described for example in U.S. Pat. Nos. 5,939,598 and 6,111,166.

The antibodies may be monospecific, bispecific, trispecific, or of greater multispecificity. A wide variety of multispecific recombinant antibody formats have been developed, for example, tetravalent bispecific antibodies by fusion of, e.g., an IgG antibody format and single chain domains (see e.g., Coloma, M. J., et al., Nature Biotech 15 (1997) 159-163; WO 2001/077342; and Morrison, S. L., Nature Biotech 25 (2007) 1233-1234). In some embodiments, the antibody or antigen-binding fragment thereof is monospecific. In some embodiments, the antibody or antigen-binding fragment thereof is bispecific. In some embodiments, the antibody or antigen-binding fragment thereof is trispecific. In some embodiments, the antibody or antigen-binding fragment thereof is multispecific. In some embodiments, the antibody or antigen-binding fragment thereof is bivalent, trivalent, tetravalent, pentavalent, or more, wherein the valency refers to the number of antibody antigen binding sites. In some embodiments, the monospecific, bispecific, trispecific or multispecific antibody or antigen-binding fragment thereof is a chimeric antibody or antigen-binding fragment thereof. In some embodiments, the monospecific, bispecific, trispecific or multispecific antibody or antigen-binding fragment thereof is a recombinant antibody or antigen-binding fragment thereof produced by techniques well-known to one of skill in the art.

Several other formats have been developed wherein the antibody core structure (IgA, IgD, IgE, IgG or IgM) is no longer retained such as diabodies, triabodies or tetrabodies, minibodies, several single chain formats (scFv, Bis-scFv), which are capable of binding two or more antigens, have been developed (Holliger, P., et al, Nature Biotech 23 (2005) 1126-1136; Fischer, N., and Léger, O., Pathobiology 74 (2007) 3-14; Shen, J., et al., Journal of immunological Methods 318 (2007) 65-74; Wu, C., et al., Nature Biotech. 25 (2007) 1290-1297). In some embodiments, the antibody or antigen-binding fragment thereof is a single chain fragment variable (scFv). In some embodiments, the antibody or antigen-binding fragment thereof is a bispecific (Bis)-scFv. In some embodiments, the antibody or antigen-binding fragment thereof is a diabody, triabody or tetrabody. In some embodiments, the antibody or antigen-binding fragment thereof is a diabody. In some embodiments, the antibody or antigen-binding fragment thereof is a triabody. In some embodiments, the antibody or antigen-binding fragment thereof is a tetrabody. In some embodiments, the antibody or antigen-binding fragment thereof is a minibody, wherein the minibody is bispecific and comprises two Fab regions (Fab₂).

More recently, camelids and sharks were discovered to possess unconventional antibodies in their immune system, known as camelid heavy-chain antibodies (HCAbs) and immunoglobulin new antigen receptors (IgNARs) respectively, reviewed in Cheong et al., Int J Macromol, 2020; 147:369-375. IgNARs are homodimers that are naturally devoid of light chain, where each heavy chain consists of five constant domains followed by one variable domain. VNARs contain autonomous function as single-domain antibodies, which are promising therapeutic candidates due to their small size, high specificities for a cognate antigen, and high physiochemical stability. VNARs have structural diversity in their antigen binding sites and are capable of accessing more cryptic epitopes and catalytic clefts of enzymes. In some embodiments, the antibody or antigen-binding fragment thereof is a single-domain antibody. In some embodiments, the antibody or antigen-binding fragment suitable for use in the ADCs of the present disclosure is a variable domain of an IgNAR (VNAR).

The variable domain of an IgNAR (i.e., the VNAR), contains only two complementarity-determining regions (CDRs), CDR1 and CDR3. Diversity of VNAR is achieved by long variable protruding CDR3, while the CDR1 is connected through two hypervariable regions (HV), HV2 and HV4. The structure of CDR3 combined with supporting intermolecular disulfide bridging confers the VNAR domain unique access to confined epitopes. Without a CDR2, VNARs are the smallest naturally occurring immunoglobulin-based protein scaffolds (roughly 12 kDa), providing a simple, highly stable scaffold useful in targeting new and difficult antigens. VNARs are categorized into four isotypes based on the position and the number of non-canonical cysteine residues.

Non-limiting examples of antibody-like backbones that may be used in an ADC according to the invention include monospecific and bispecific fragments such as multimerizing scFv fragments (diabodies, triabodies, tetrabodies), disulfide stabilized antibody variable (Fv) fragments, disulfide stabilized antigen-binding (Fab) fragments consisting of the VL, VH, CL and CH1 domains, bivalent F(ab′)2 fragments, Fd fragments consisting of the heavy chain and CH1 domains, dimeric CH2 domain fragments (CH2D), FC antigen binding domains (Fcabs), single chain FV-CH3 minibodies, bispecific minibodies, isolated complementary determining region 3 (CDR3) fragments, constrained FR3-CDR3-FR4 polypeptides, SMTP domains, variable domains of these shark antibodies (VNARs) and any genetically manipulated counterparts of the foregoing

All such antibody or antigen-binding fragment formats use linkers either to fuse the antibody core (IgA, IgD, IgE, IgG or IgM) to a further binding protein (e.g. scFv) or to fuse, for example, two Fab fragments or scFvs (Fischer, N., and Leger, O., Pathobiology 74 (2007) 3-14). The antibody or antigen-binding fragment thereof may be engineered to retain effector functions, such as, for example, complement-dependent cytotoxicity (CDC) or antibody dependent cellular cytotoxicity (ADCC), which are mediated through the receptor binding, by maintaining a high degree of similarity to naturally occurring antibodies.

In some embodiments, the antibody or antigen-binding fragment thereof is directed to a target antigen. The antibody or antigen-binding fragment thereof may be directed against any target antigen of interest, such as of medical and/or therapeutic interest. For example, the antigen can be one associated a particular medical condition, such as cancer. In the case of a tumor-associated antigen (TAA), the cancer may be of the immune system, lung, colon, rectum, breast, ovary, prostate gland, head, neck, bone, or any other anatomical location. Antigens of interest include, but are not limited to, PSMA, CD33, HER2, Trop-2, HER3, B7H3, B7H4, CEACAM5, MET, NECTIN4, CALR, or CD123.

Useful antibodies include polyclonal antibodies, which are heterogeneous populations of antibody molecules derived from the sera of immunized animals. Other useful antibodies are monoclonal antibodies, which are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The antibody can be a functionally active fragment, derivative or analog of an antibody that immunospecifically binds to targeted cells (e.g., cancer cell antigens, viral antigens, or microbial antigens) or other antibodies bound to tumor cells or matrix. In this regard, “functionally active” means that the fragment, derivative or analog is able to immunospecifically bind to target cells. To determine which CDR sequences bind the antigen, synthetic peptides containing the CDR sequences can be used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Other useful antibodies include fragments of antibodies such as, but not limited to, F(ab′)₂ fragments, Fab fragments, Fvs, single chain antibodies, diabodies, triabodies, tetrabodies, scFv, scFv-FV, bis-scFv, single domain antibodies or any other molecule with the same specificity as the antibody. Engineered antibodies that are multivalent and/or multispecific can also be used, for example, a tetravalent bispecific antibody or any other molecule engineered antibody described herein.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety). Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety). Such chimeric and humanized monoclonal antibodies can be produced by recombinant DNA techniques known in the art, for example using methods, each of which is specifically incorporated herein by reference, as described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., Science (1988) 240:1041-1043; Liu et al., Proc. Natl. Acad. Sci. (USA) (1987) 84: 3439-3443; Liu et al., J. Immunol. (1987) 139: 3521-3526; Sun et al. Proc. Natl. Acad. Sci. (USA) (1987) 84: 214-218; Nishimura et al. Cancer. Res. (1987) 47: 999-1005; Wood et al., Nature (1985) 314:446-449; Shaw et al., J. Natl. Cancer Inst. (1988) 80: 1553-1559; Morrison, Science (1985) 229:1202-1207; Oi et al. BioTechniques (1986) 4: 214; U.S. Pat. No. 5,225,539; Jones et al., Nature (1986) 321: 552-525; Verhoeyan et al., Science (1988) 239: 1534; and Beidler et al., J. Immunol. (1988) 141: 4053-4060.

Completely human antibodies can be produced using transgenic mice or other transgenic animals that are incapable of expressing endogenous immunoglobulin heavy and light chains genes, but which can express human heavy and light chain genes.

Antibodies include analogs and derivatives that are either modified, i.e., by the covalent attachment of any type of molecule if such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivitization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Further, associated heterologous molecules which may be fused, linked, joined covalently or non-covalently, or otherwise engineered together in accordance with the disclosure may comprise, e.g., one or more biologically active molecules and/or imaging agents. Exemplary biologically active molecules include, e.g., toxins for targeted cell death (useful e.g., in certain hyperproliferative diseases or disorders such as cancers or aberrant proliferative conditions). Other exemplary biologically active molecules which may be used in association with the antibody or antigen-binding fragment thereof of the ADCs of the disclosure include, e.g., polypeptides, such as an antibody or antibody fragment; a therapeutic peptide such as a hormone, cytokine, growth factor, enzyme, antigen or antigenic peptide, transcription factor, or any functional domain thereof. Other exemplary biologically active molecules which may be used in association with the antibody or antigen-binding fragment thereof suitable for use in the ADCs of the disclosure include, e.g., nucleic acid molecules, such as an oligonucleotide (e.g., single, double or more stranded RNA and/or DNA molecules, and analogs and derivatives thereof); small regulatory RNA such as shRNA, miRNA, siRNA and the like; and a plasmid or fragment thereof.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor (see, e.g., International Publication No. WO 97/34631, which is incorporated herein by reference in its entirety).

In specific embodiments, known antibodies for the treatment of cancer are used. In some embodiments, the antibody will selectively bind to a cancer antigen of a hematological malignancy.

In some embodiments, the antibody or antigen-binding fragment thereof is a full-length antibody, a Fab, a Fab′, a (Fab′)₂, an Fv, or a single chain Fv (scFv). In some embodiments, the antibody or antigen-binding fragment thereof is a half antibody. In some embodiments, the antibody is a full-length antibody. In some embodiments, the antibody is an asymmetric antibody. In some embodiments, the antibody comprises at least one heavy chain variable domain (VH) and at least one light chain variable domain (VL). In some embodiments, the heavy chain variable domain comprises three heavy chain complementarity determining regions (CDR-H1-3). In some embodiments, the light chain variable domain comprises three light chain complementarity determining regions (CDR-L1-3).

Exemplary antigens for the antibodies are provided below. Exemplary antibodies that bind the indicated antigen are shown in parentheses.

In some embodiments, the antigen (e.g., the antigen for the antibody) is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD147 (exemplary antibodies include gavilimomab and metuzumab), CD19, CD20 (exemplary antibodies include divozilimab and ibritumomab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab, gemtuzumab, and vadastuximab), CD352, CD45 (exemplary antibodies include apamistamab), CD47 (exemplary antibodies include letaplimab and magrolimab), CLPTM1L, CD205 also known as LY75 and CLEC13B, DPP4, EGFR, ERVMER34-1, FASL, FSHR, FZD5, FZD8, GUCY2C (exemplary antibodies include indusatumab), IFNA1 (exemplary antibodies include faralimomab), IFNAR1, IFNAR2, LMP2, MHLANA, SIT1, TLR2/4/1 (exemplary antibodies include tomaralimab), TM4SF5, TMEM132A, TMEM40, UPK1B, VGFR1, VGRF2, and VEGFR2 (exemplary antibodies include gentuximab).

In some embodiments, the tumor-associated antigen is a growth factor protein. For example, the following antigen is a growth factor protein: VEGF (exemplary antibodies include bevacizumab).

In some embodiments, the tumor-associated antigen is a transmembrane transport protein. For example, the following antigens are transmembrane transport proteins: ASCT2 (exemplary antibodies include idactamab), MFSD13A, Mincle, NOX1, SLC10A2, SLC12A2, SLC17A2, SLC38A1, SLC34A2, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD 112, CD155, CD24, CD247, CD37 (exemplary antibodies include lilotomab), CD38 (exemplary antibodies include felzartamab and daratumumab), CD3D, CD3E (exemplary antibodies include foralumab and teplizumab), CD3G, CD96, CDCP1, CDH17, CDH3, CDH6, CEACAM1, CEACAM6, CLDN1, CLDN6, CLDN16, CLDN18.1 (exemplary antibodies include zolbetuximab), CLDN18.2 (exemplary antibodies include zolbetuximab), CLDN19, CLDN2, DPEP1, DPEP3, DSG2, endosialin (exemplary antibodies include ontuxizumab), ENPP1, EPCAM (exemplary antibodies include adecatumumab), FN, FN1, Gp100, Globo H, GPA33, gpNMB (exemplary antibodies include glembatumumab), ICAM1, L1CAM, LAMP1, MELTF also known as CD228, NCAM1, Nectin-4 (exemplary antibodies include enfortumab), PDPN, PROM1, PSCA, PSMA (exemplary antibodies include J591/MLN591 and rosopatamab), Siglecs 1-16, SIRPa, SIRPg, TACSTD2 also know as TROP2 (exemplary antibodies include sacituzumab and datoptamab), TAG-72, Tenascin, Tissue Factor also known as TF (exemplary antibodies include tisotumab), ULBP1/2/3/4/5/6, and 5T4.

In some embodiments, the tumor-associated antigen is a transmembrane or membrane-associated receptor kinase. For example, the following antigens are transmembrane or membrane-associated receptor kinases: ALK, AXL (exemplary antibodies include tilvestamab and enapotamab), BMPR2, DCLK1, DDR1, EPHA receptors, EPHA2, ERBB2 also known as HER2 (exemplary antibodies include trastuzumab, pertuzumab, margetuximab, and disitamab), ERBB3, FLT3, MET (exemplary antibodies include telisotuzumab), IGF1R, PDGFR-B (exemplary antibodies include rinucumab), PTK7 (exemplary antibodies include cofetuzumab), RET, ROR1 (exemplary antibodies include cirmtuzumab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab and mirvetuximab), IL13Ra2, IL1RAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS1R.

In some embodiments, the tumor-associated antigen is cell-surface-associated or a cell-surface receptor. For example, the following antigens are cell-surface-associated and/or cell-surface receptors: B7-DC, BCMA, CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3, GITR (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, OY-TES1, PLEC, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD 115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, ITGAV (exemplary antibodies include abituzumab), ITGB6, and ITGB8.

In some embodiments, the tumor-associated antigen is a glycolipid. For example, the following are glycolipid antigens: FucGM1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM12, ADAM9, TMPRSS11D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C₅ complement, CCNB1, CSPG4, CYP1B1, De2-7 EGFR, EGF, Fas-related antigen 1, FBP, G250, GAGE, HAS3, HPV E6 E7, hTERT, IDO1, LCK, Legumain, LYPD1, MAD-CT-1, MAD-CT-2, MAGEA3, MAGEA4, MAGEC2, MerTk, ML-IAP, NA17, NY-BR-1, p53, p53 mutant, PAP, PLAVI, polysialic acid, PR1, PSA, Sarcoma translocation breakpoints, SART3, sLe, SSX2, Survivin, Tn, TRAIL, TRAIL1, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD163, CD19, CD20 (exemplary antibodies include rituximab, ocrelizumab, divozilimab; ibritumomab), CD22, CD25 (exemplary antibodies include basiliximab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD46, CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SIT1, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD 112, CD155, CD24, CD247, CD28, CD30L, CD37 (exemplary antibodies include lilotomab), CD38 (exemplary antibodies include felzartamab and daratumumab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: AXL (exemplary antibodies include tilvestamab and enapotamab) and FLT3.

In some embodiments, the immune-cell-associated antigen is a membrane-associated or membrane-localized protein. For example, the following antigens are membrane-associated or membrane-localized proteins: CD83, IL1RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR7, CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD71, CD74 (exemplary antibodies include milatuzumab), CD79A, CD262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD 115 (exemplary antibodies include axatilimab, cabiralizumab, and emactuzumab), CD123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

### Linkers (L)

In some embodiments, a “linker” (L) is a chemical moiety that covalently links an antibody, or an antigen-binding fragment thereof, to a degrader compound to form an Antibody Drug Conjugate compound as described herein. For example, in some embodiments, a cysteine thiol of an antibody, or an antigen-binding fragment thereof, can form a covalent bond with a reactive group of a linker moiety.

In some embodiments, a “linker” (L) is a chemical moiety that is covalently bonded to a degrader compound to form a Degrader-Linker compound as described herein.

In some embodiments, the linker comprises one or more cleavable moieties. In some embodiments, the cleavable moiety is susceptible to cleavage under certain conditions. Cleavage of the cleavable moiety of an Antibody Drug Conjugate compound as described herein results in the release of the degrader compound as a free drug.

In some embodiments, the cleavable moiety comprises a peptide group. In some embodiments, the peptide group is susceptible to cleavage by an intracellular or regulatory protease but remains stable in plasma. In some embodiments, the peptide moiety comprises one or more amino acids. In some embodiments, the peptide moiety is a dipeptide. In some embodiments, the peptide moiety is a tripeptide. In some embodiments, the peptide moiety is a tetrapeptide.

In some embodiments, the cleavable moiety comprises a disulfide group. In some embodiments, the disulfide group is susceptible to cleavage in a reducing environment, such as the cytoplasm of a cell but remains stable in plasma. In some embodiments, the disulfide group is cleaved by the reducing environment caused by the concentration of intracellular glutathione.

In some embodiments, the cleavable moiety comprises an acid-labile group. In some embodiments, the acid-labile group is susceptible to hydrolysis in mildly acidic environments, such as weakly acidic endosomes (e.g., pH 5.0-6.5) or lysosomes (pH 4.5-5.0), but remains stable in neutral environments, such as blood (pH 7.3-7.5). In some embodiments, the acid-labile group is a hydrazine. In some embodiments, the acid-labile group is carbonate. In some embodiments, the acid-labile group is acetal. In some embodiments, the acid-labile group is ketal.

In some embodiments, L is a linker of Formula (II):

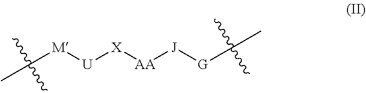

wherein,
M is selected from the group consisting of:

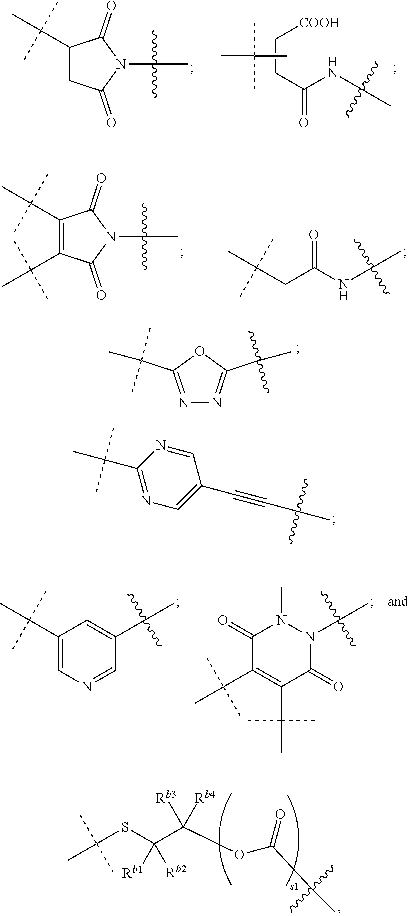

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1,
   wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or
   —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
      —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
      -heterocycloalkyl-O(CH₂)ᵢC(O)—, wherein subscript i is an integer ranging from 0 to 6;
      —C(O)—C(R^{x5})(R^{x6})—OC(O)—, wherein R^{x5} is hydrogen or C₁-C₆ alkyl and R^{x6} is hydrogen, C₁-C₆ alkyl, or

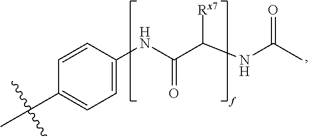

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
AA is absent or has the structure of:

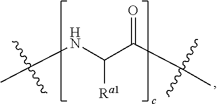

wherein subscript c is an integer ranging from 1 to 12; each R^{a1} is independently —COOH, —NH₂, or an independently selected side chain of an amino acid;
J is absent, —(R^{j3})N(C(R^{j1})(R^{j2}))ₘ₁—, or has the structure of:

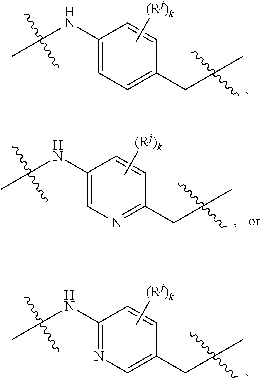

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
G is absent,

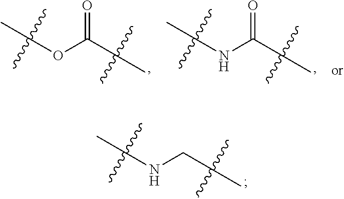

and
   wherein the wavy line to M of Formula (II) indicates the point of covalent attachment to Ab and the wavy line to G of Formula (II) indicates the point of covalent attachment to D.

In some embodiments, M has the structure of

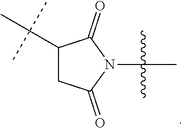

In some embodiments, M comprises a succinimide ring. In some embodiments, a carbonyl-nitrogen bond of the succinimide ring is hydrolyzed. In some embodiments, M exists in a hydrolyzed form. In some embodiments, M comprises a succinic acid. In some embodiments, M has the structure of

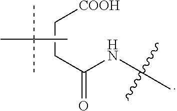

In some embodiments, M has the structure of

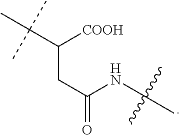

In some embodiments, M has the structure of

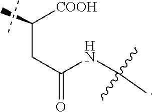

In some embodiments, M has the structure of

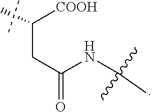

In some embodiments, M has the structure of

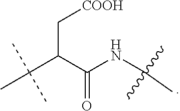

In some embodiments, M has the structure of

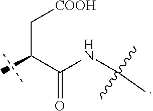

In some embodiments, M has the structure of

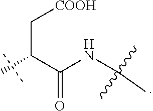

In some embodiments, the amide nitrogen corresponds to the imide nitrogen of the succinimide. It will be understood that where an ADC has a succinimide group connected to an antibody, the succinimide group may also exist in a ring-opened form.

In some embodiments, M has the structure of:

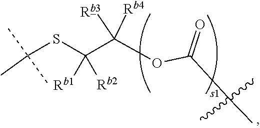

wherein
R^{b1} and R^{b2} are each independently hydrogen or C₁-C₆ alkyl, or R^{b1} and R^{b2}together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1,
wherein the dashed line indicates the point of covalent attachment to the Ab and the wavy line indicates the point of covalent attachment to the remainder of the structure of L.

In some embodiments, M has the structure of:

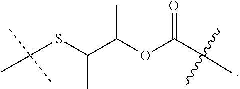

In some embodiments, M has the structure of:

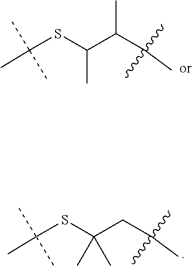

In some embodiments, M has the structure of:

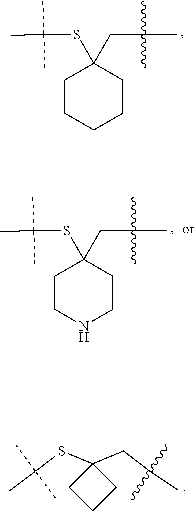

In some embodiments, U is absent or is —(CH₂)_{b}(R^{v1})ₛₛ(C═O)ᵤ(NH)ᵥ—, wherein subscript b is 0, 1, 2, 3, 4, or 5; subscript ss is 0 or 1; subscript u is 0 or 1; subscript v is 0 or 1; and R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl. In some embodiments, U is absent.

In some embodiments, U is present is and is —(CH₂)_{b}(R^{v1})ₛₛ(C═O)ᵤ(NH)ᵥ—, wherein subscript b is 1, 2, 3, 4, or 5; subscript ss is 0; subscript u is 0 or 1; subscript v is 0 or 1. In some embodiments, U has the structure of:

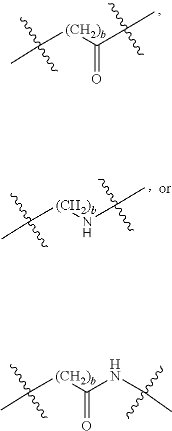

wherein
subscript b is 1, 2, 3, 4, or 5, the dashed line indicates the point of covalent attachment to M, and the wavy line indicates the point of covalent attachment to the remaining structure of L.

In some embodiments, U has the structure of:

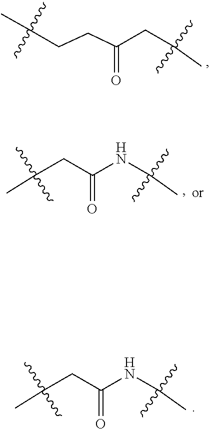 [SMILES: *CCC(=O)C* ; *CC(=O)N* ; *CC(=O)N*]

In some embodiments, U has the structure of:

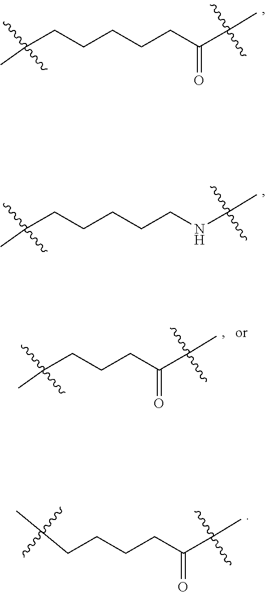 [SMILES: *CCCCCC(*)=O ; *CCCCCN* ; *CCCC(*)=O ; *CCCCC(*)=O]

In some embodiments, U is present and is —(CH₂)_{b}(R^{v1})ₛₛ(C═O)ᵤ(NH)ᵥ—, wherein subscript b is 0, 1, 2, 3, 4, or 5; subscript ss is 1; subscript u is 0 or 1; subscript v is 0 or 1; and R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl.

In some embodiments, U has the structure of:

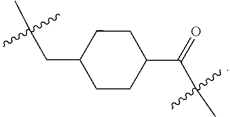 [SMILES: *CC1CCC(C(*)=O)CC1]

In some embodiments, U is present and is —(CH₂)_{b}(R^{v1})ₛₛ(C═O)ᵤ(NH)ᵥ—, wherein subscript b is 0, 1, 2, 3, 4, or 5; subscript ss is 1; subscript u is 0 or 1; subscript v is 0 or 1; and R^{v1} is —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl. In some embodiments, U has the structure of:

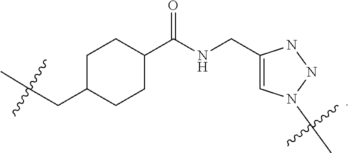 [SMILES: *CC1CCC(C(=O)NCC2=CN(*)NN2)CC1]

In some embodiments, U is present and is —(CH₂CH₂O)_{w}CH₂CH₂(NH)—, wherein subscript w is an integer ranging from 1 to 16. In some embodiments, subscript w is an integer ranging from 1 to 12. In some embodiments, subscript w is an integer ranging from 2 to 10. In some embodiments, subscript w is an integer ranging from 4 to 8. In some embodiments, subscript w is 1. In some embodiments, subscript w is 2. In some embodiments, subscript w is 4. In some embodiments, subscript w is 8.

In some embodiments, U has the structure of:

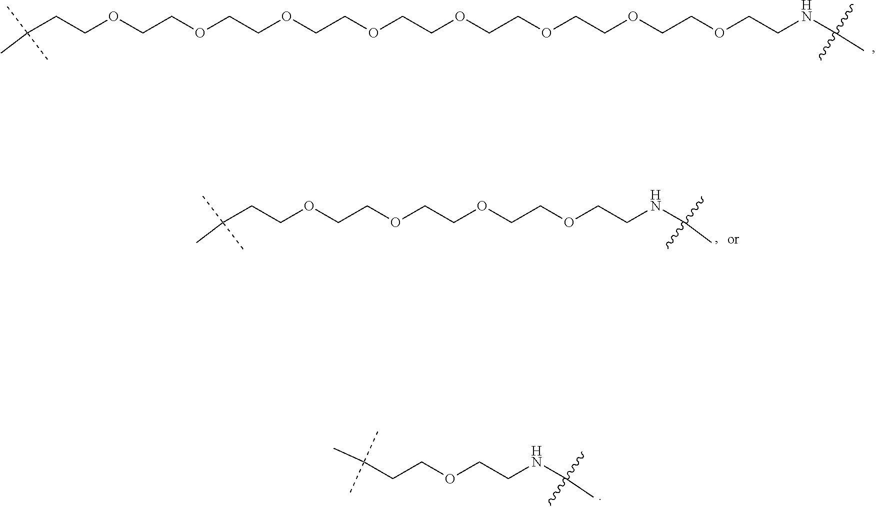

In some embodiments, X is absent.

In some embodiments, X is present and is —(CH₂CH₂O)_{w}CH₂CH₂—(C═O)_{d}—, wherein subscript w is an integer ranging from 0 to 16 and subscript d is 0 or 1. In some embodiments, subscript w is an integer ranging from 1 to 12. In some embodiments, subscript w is an integer ranging from 2 to 10. In some embodiments, subscript w is an integer ranging from 4 to 8. In some embodiments, subscript w is 1. In some embodiments, subscript w is 2. In some embodiments, subscript w is 4. In some embodiments, subscript w is 8. In some embodiments, d is 0. In some embodiments, d is 1.

In some embodiments, X has the structure of:

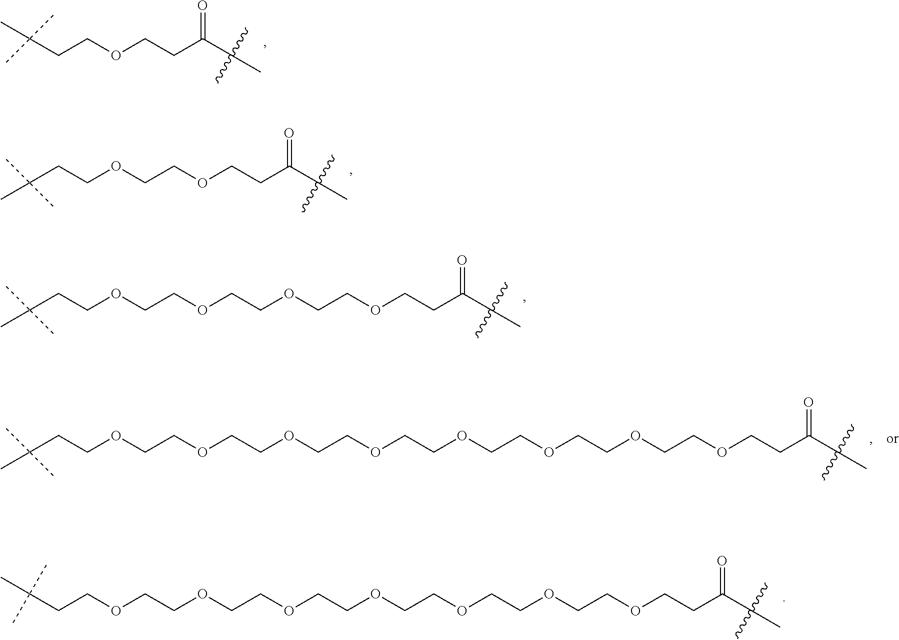

In some embodiments, X is present and is —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; and subscript x and subscript y are each independently an integer ranging from 0 to 8. In some embodiments, subscript nn is an integer ranging from 2 to 4. In some embodiments, subscript nn is 2. In some embodiments, subscript x is 5. In some embodiments, subscript x is 1. In some embodiments, subscript y is 8. In some embodiments, subscript y is 1.

In some embodiments, R^{x1} has the structure of:

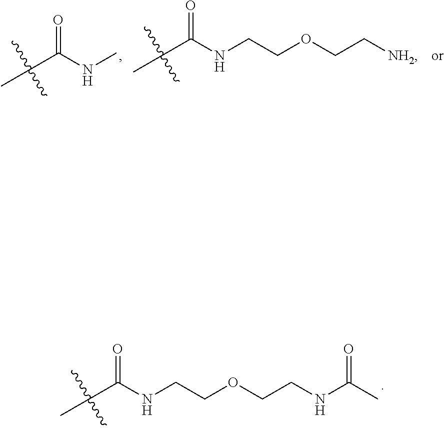

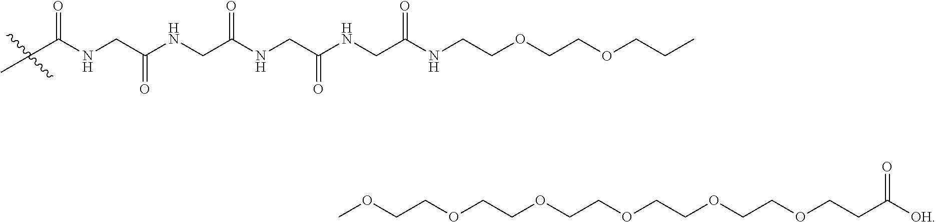

In some embodiments, R^{x1} has the structure of:

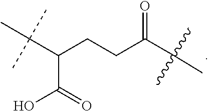

In some embodiments, X is present and is —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl.

In some embodiments, X has the structure of:

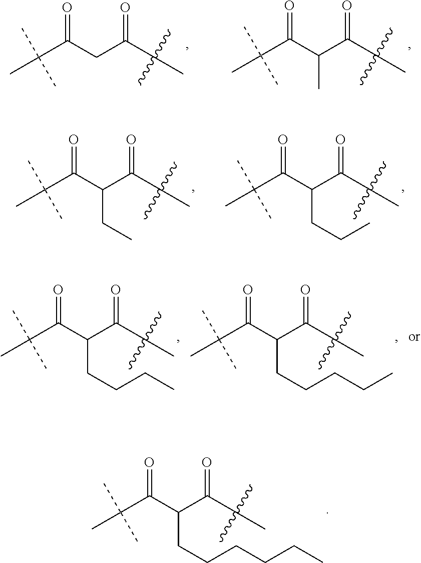

In some embodiments, X has the structure of:

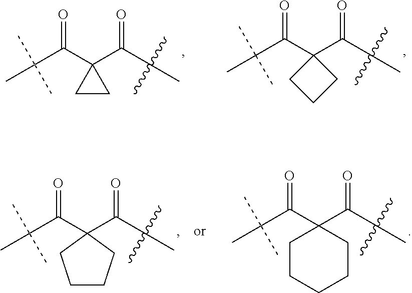

In certain embodiments, X has the structure of:

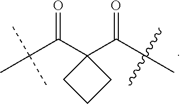

In some embodiments, X is present and is -heterocycloalkyl-O(CH₂)ᵢC(O)—, wherein subscript i is an integer ranging from 0 to 6. In some embodiments, subscript i is 1. In some embodiments, X has the structure of:

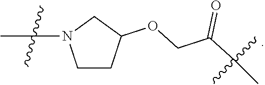 [SMILES: *C(=O)COC1CCN(*)C1]

In some embodiments, X is present and is —C(O)—C(R^{x5})(R^{x6})—OC(O)—, wherein R^{x5} is hydrogen or C₁-C₆ alkyl and R^{x6} is hydrogen, C₁-C₆ alkyl, or

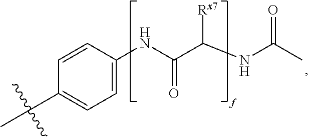

wherein subscript f is an integer ranging from 0 to 4, and each R^{x7} is independently hydrogen, C₁-C₆ alkyl, —COOH, —NH₂, or an independently selected side chain of an amino acid. In some embodiments, the amino acid is selected from the group consisting of alanine, valine, glutamic acid, citrulline, tryptophan, glycine, phenylalanine, and lysine.

In some embodiments, R^{x6} has the structure of:

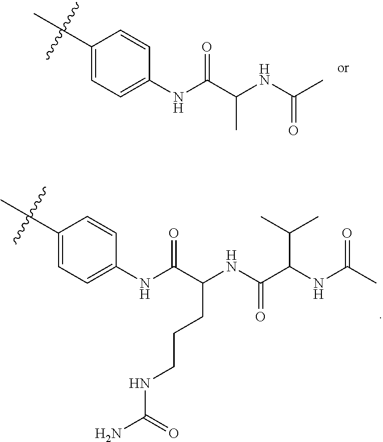 [SMILES: *c1ccc(NC(=O)C(C)NC(C)=O)cc1 ; *c1ccc(NC(=O)C(CCCNC(N)=O)NC(=O)C(NC(C)=O)C(C)C)cc1]

In some embodiments, X is —(CH₂CH₂O)_{yy}CH₂CH₂NHC(O)CH₂OCH₂C(O)—, wherein subscript yy is an integer ranging from 0 to 16. In some embodiments, yy is an integer ranging from 1 to 12. In some embodiments, yy is an integer ranging from 2 to 10. In some embodiments, yy is an integer ranging from 4 to 8. In some embodiments, yy is 8.

In some embodiments, X has the structure of:

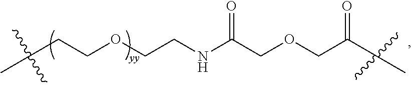

wherein yy is 8.

In some embodiments, AA is absent or has the structure of:

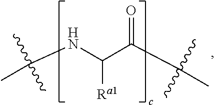

wherein subscript c is an integer ranging from 1 to 12; each R^{a1} is independently —COOH, —NH₂, or an independently selected side chain of an amino acid. In some embodiments, each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine.

In some embodiments, AA is present and subscript c in an integer ranging from 1 to 10. In some embodiments, subscript c is an integer ranging from 1 to 8. In some embodiments, subscript c is an integer ranging from 1 to 6. In some embodiments, subscript c is an integer ranging from 1 to 4. In some embodiments, subscript c is 1, 2, 3, 4, or 5. In some embodiments, subscript c is 1. In some embodiments, subscript c is 2. In some embodiments, subscript c is 3. In some embodiments, subscript c is 4.

In some embodiments, c is 1 and R^{a1} is —COOH, —NH₂, or an independently selected side chain of an amino acid selected from the group consisting of alanine, valine, glutamic acid, citrulline, tryptophan, glycine, phenylalanine, and lysine. In some embodiments, c is 1 and R^{a1} is citrulline.

In some embodiments, c is 2 and each R^{a1} is —COOH, —NH₂, or an independently selected side chain of an amino acid, wherein each amino acid is selected from the group consisting of alanine, valine, glutamic acid, citrulline, tryptophan, glycine, phenylalanine, and lysine. In some embodiments, c is 2 and R^{a1} is valine and alanine. In some embodiments, c is 2 and R^{a1} is valine and glycine. In some embodiments, c is 2 and R^{a1} is valine and citrulline. In some embodiments, c is 2 and R^{a1} is tryptophan and citrulline. In some embodiments, c is 2 and R^{a1} is phenylalanine and citrulline.

In some embodiments, c is 3 and each R^{a1} is —COOH, —NH₂, or an independently selected side chain of an amino acid, wherein each amino acid is selected from the group consisting of alanine, valine, glutamic acid, citrulline, tryptophan, glycine, phenylalanine, and lysine. In some embodiments, c is 3 and R^{a1} is glutamic acid, glycine, and citrulline.

In some embodiments, c is 4 and each R^{a1} is —COOH, —NH₂, or an independently selected side chain of an amino acid, wherein each amino acid is selected from the group consisting of alanine, valine, glutamic acid, citrulline, tryptophan, glycine, phenylalanine, and lysine. In some embodiments, c is 4 and R^{a1} is glycine, glycine, phenylalanine, and glycine.

In some embodiments, AA has the structure of:

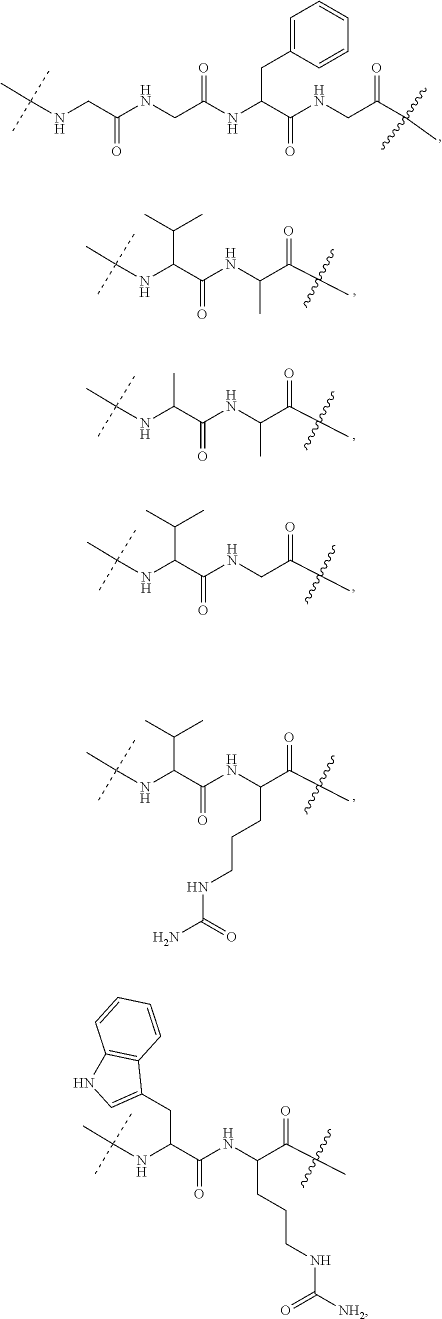

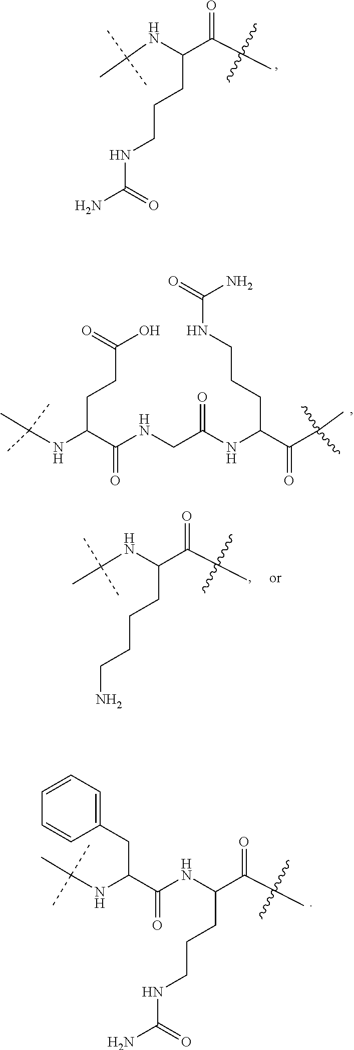

In some embodiments, J is absent, —(R^{j3})N(C(R^{j1})(R^{j2}))ₘ₁—, or has the structure of:

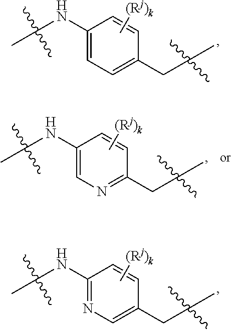

wherein R^{j3} is hydrogen, C₁-C₆ alkyl, or C₃-C₆ cycloalkyl; each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl; subscript m1 is an integer ranging from 1 to 6; each R³ is independently halogen, C₁-C₆ alkyl, or —C(O)NH(C₁-C₆ alkyl); and subscript k is an integer ranging from 0 to 4.

In some embodiments, R^{j} is halogen. In some embodiments, R^{j} is fluorine. In some embodiments, R^{j} is chlorine. In some embodiments, R^{j} is fluorine. In some embodiments, R^{j} is C₁-C₆ alkyl. In some embodiments, R^{j} is methyl. In some embodiments, R^{j} is —C(O)NH(Cf₁—C₆ alkyl). In some embodiments, R^{j} is —C(O)NHCH₃. In some embodiments, R^{j} is methyl, —F, —Cl, or —C(O)NHCH₃.

In some embodiments, J has the structure of:

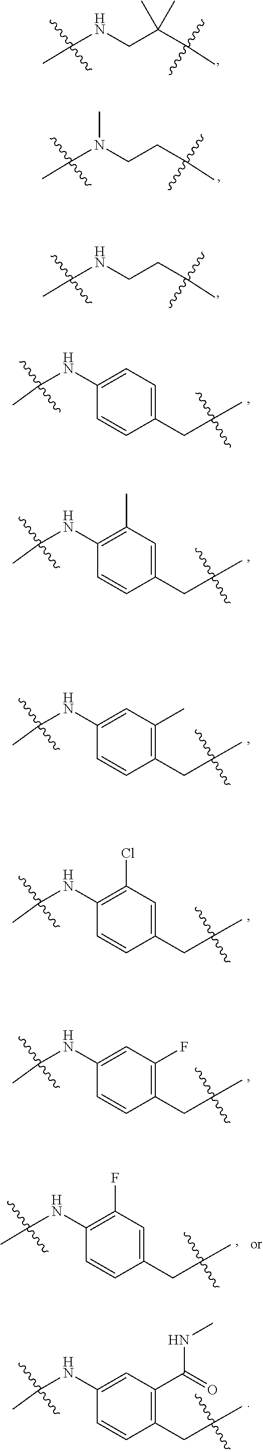

In some embodiments, G is absent,

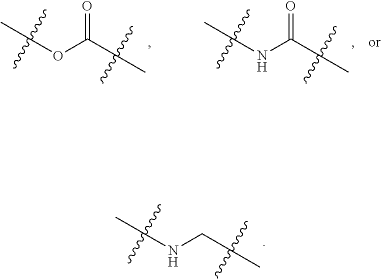

In some embodiments, G is

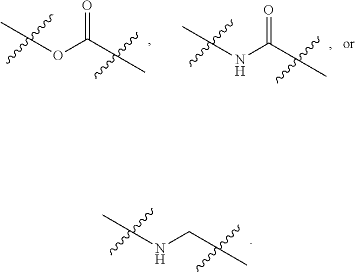

In some embodiments, G is absent. In some embodiments, G is

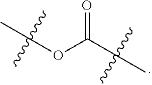 [SMILES: *OC(*)=O]

In some embodiments, G is

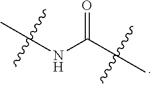 [SMILES: *NC(*)=O]

In some embodiments, G is

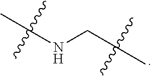 [SMILES: *CN*]

In some embodiments, the wavy line to M of Formula (II) indicates the point of covalent attachment to Ab and the wavy line to G of Formula (II) indicates the point of covalent attachment to D. In some embodiments, the wavy line to G of Formula (II) indicates the point of covalent attachment to a secondary alcohol group or a secondary amine group of D. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, L is a linker of Formula (IIa):

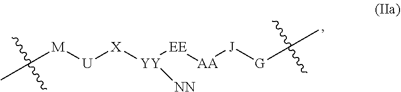

wherein
M is selected from the group consisting of:

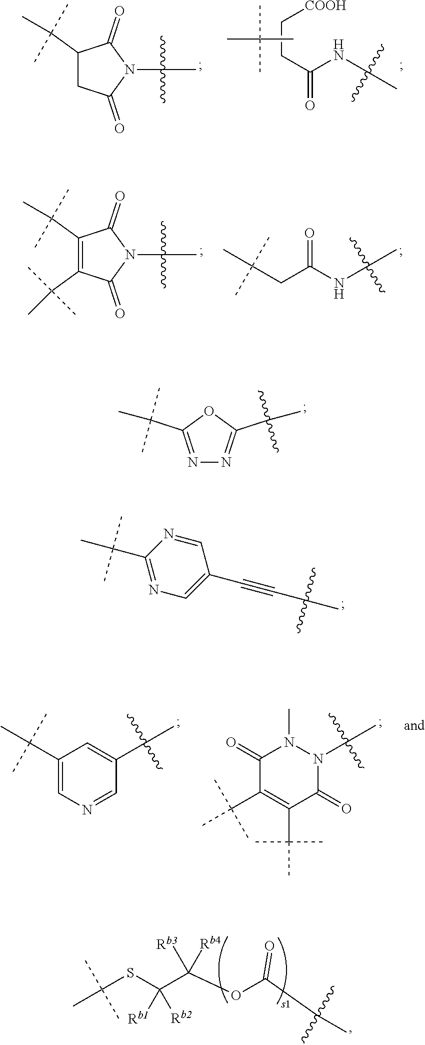

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1,
   wherein the dashed line indicates the point of covalent attachment to the
Ab and the wavy line indicates the point of covalent attachment to the remainder of the structure of L;
U is absent or is —(CH₂)_{b} (R^{v1})ₛₛ(C═O)ᵤ(NH)ᵥ—, wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6, and subscript x and subscript y are each independently an integer ranging from 0 to 8;
   —C(O)—C(R^{x3})(R^{x4})—C(O)—, wherein R^{x3} and R^{x4} are each independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript I is an integer ranging from 0 to 6;
   —C(O)—C(R^{x5})(R^{x6})—OC(O)—, wherein R^{x5} is hydrogen or C₁-C₆ alkyl, R^{x6} is hydrogen, C₁-C₆ alkyl, or

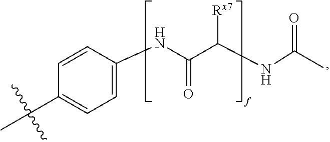

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit;
NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
EE is absent or —(CH₂CH₂O)_{x′}CH₂CH₂C(O)—, wherein subscript x′ is an integer ranging from 0 to 16;
AA is absent or has the structure of:

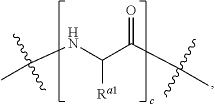

wherein subscript c is an integer ranging from 1 to 12; each R^{a1} is independently —COOH, —NH₂, or an independently selected side chain of an amino acid;
J is independently absent, —(R^{j3})N(C(R^{j1})(R^{j2}))ₘ₁—, or has the structure of:

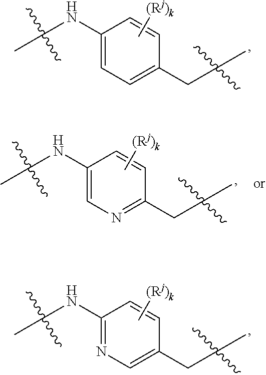

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
G is absent,

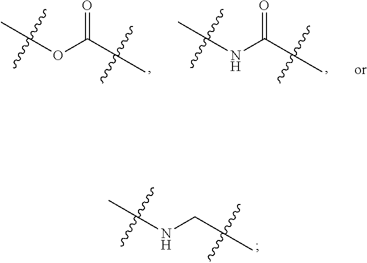

and
wherein the wavy line to M of Formula (IIa) indicates the point of covalent attachment to Ab and the wavy line to G of Formula (IIa) indicates the point of covalent attachment to D.

In some embodiments, YY is a branching unit selected from the group consisting of:

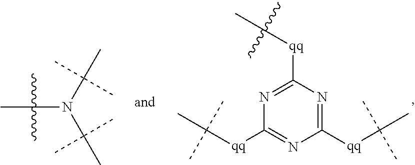

wherein each qq is independently —N(R^{y1})— or —O—, wherein R^{y1} is hydrogen or C₁-C₆ alkyl, wherein one dashed line indicates the point of covalent attachment to EE when EE is present, or to AA when EE is absent, or to J when AA and EE are absent, or to G when AA, EE, and J are absent, and the other dashed line indicates the point of covalent attachment to NN, wherein the wavy line indicates the point of covalent attachment to X, when X is present, or to U, when X is absent, or to M, when X and U are absent.

In some embodiments, the branching unit (YY) is an at least tri-substituted carbocycle, an at least tri-substituted heterocycle, a tertiary carbon atom, or a tertiary nitrogen atom. In some embodiments, the branching unit comprises at least three functional groups independently selected from the group consisting of an amine, a carboxylate, a thiocarboxylate, hydroxyl, thiol, carbamate, thiocarbamate, sulfonate, sulfonamide, phosphonate, and phosphinate.

In some embodiments, YY is a branching unit selected from the group consisting of:

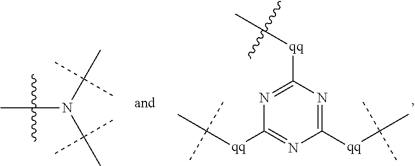

wherein each qq is independently —N(R^{y1})— or —O—, wherein R^{y1} is hydrogen or C₁-C₆ alkyl. In some embodiments, the branching unit (YY) is

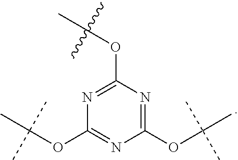

In some embodiments, the branching unit (YY) is

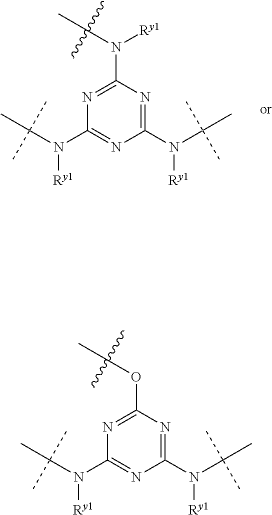

and each R^{y1} is independently hydrogen or C₁-C₆ alkyl. In some embodiments, R^{y1} is hydrogen. In some embodiments, R^{y1} is C₁ alkyl. In some embodiments, R^{y1} is C₂ alkyl. In some embodiments, R^{y1} is C₃ alkyl. In some embodiments, R^{y1} is C₄ alkyl. In some embodiments, R^{y1} is C₅ alkyl. In some embodiments, R^{y1} is C₆ alkyl. In some embodiments, the
branching unit (YY) is

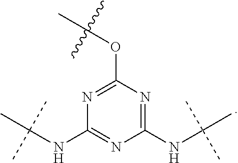

In some embodiments, the wavy line to M of Formula (IIa) indicates the point of covalent attachment to Ab and the wavy line to G of Formula (IIa) indicates the point of covalent attachment to D. In some embodiments, the wavy line to G of Formula (IIa) indicates the point of covalent attachment to a secondary alcohol group or a secondary amine group of D. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, L is a linker of Formula (IIb):

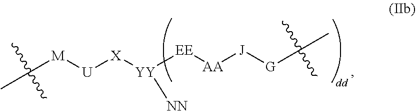

wherein
subscript dd is 1 or 2;
M is selected from the group consisting of:

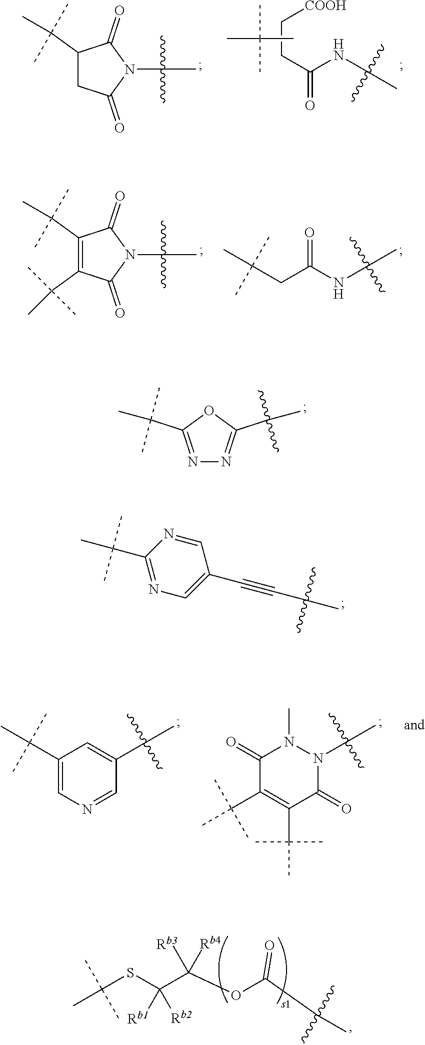

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1,
   wherein the dashed line indicates the point of covalent attachment to the Ab and the wavy line indicates the point of covalent attachment to the remainder of the structure of L;
   U is absent or is —(CH₂)_{b} (R^{v1})ₛₛ(C═O)ᵤ(NH)ᵥ—, wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6, and subscript x and subscript y are each independently an integer ranging from 0 to 8;
   —C(O)—C(R^{x3})(R^{x4})—C(O)—, wherein R^{x3} and R^{x4} are each independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript I is an integer ranging from 0 to 6;
   —C(O)—C(R^{x5})(R^{x6})—OC(O)—, wherein R^{x5} is hydrogen or C₁-C₆ alkyl, R^{x6} is hydrogen, C₁-C₆ alkyl, or

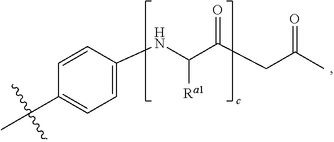

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit, wherein
   when subscript dd is 1, YY has the structure of:

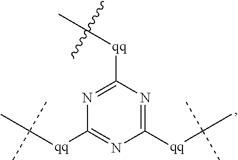

wherein
   each qq is independently —N(R^{y1})— or —O—, wherein R^{y1} is hydrogen or C₁-C₆ alkyl, and
wherein subscript dd is 2, YY has the structure of:

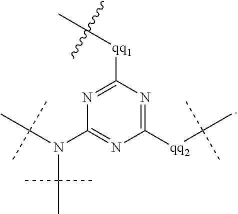

wherein
   qq₁ and qq₂ are each independently —N(R^{y1})— or —O—, wherein R^{y1} is hydrogen or C₁-C₆ alkyl, and
   the wavy line of YY indicates the point of attachment to X, when present, or U, when X is absent, or M, when X and U are absent; and
   the dashed lines of YY indicate the point of attachment to EE when EE is present, or to AA when EE is absent, or to J when AA and EE are absent, or to G when AA, EE, and J are absent, and to NN;
NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
each EE is independently absent or —(CH₂CH₂O)_{x′}CH₂CH₂C(O)—, wherein subscript x′ is an integer ranging from 0 to 16;
each AA is independently absent or has the structure of:

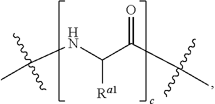

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

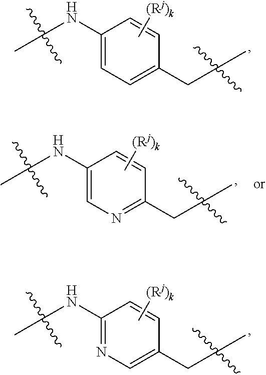

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
each G is independently absent,

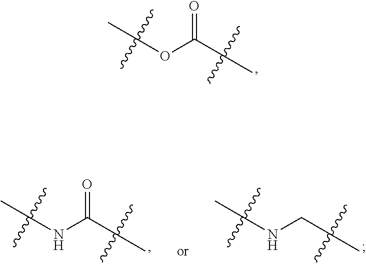

and
wherein the wavy line to M of Formula (IIb) indicates the point of covalent attachment to Ab and the wavy line to G of Formula (IIb) indicates the point of covalent attachment to an independently selected D.

In some embodiments, qq₁ and qq₂ are each —N(R^{y1})—, wherein R^{y1} is hydrogen or C₁-C₆ alkyl. In some embodiments, qq₁ and qq₂ are each —O—. In some embodiments, qq₁ is —N(R^{y1})—, wherein R^{y1} is hydrogen or C₁-C₆ alkyl, and qq₂ is —O—. In some embodiments, R^{y1} is hydrogen.

In some embodiments, YY is:

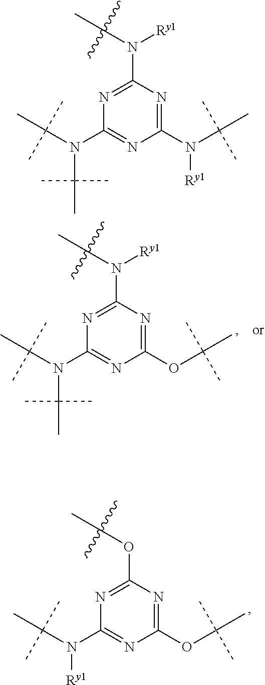

wherein R^{y1} is hydrogen or C₁-C₆ alkyl.

In some embodiments, the linker of Formula (IIb) has the structure of:

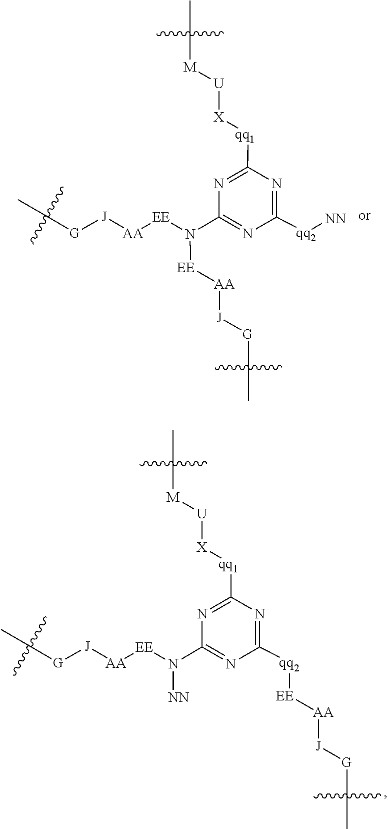

wherein qq₁ and qq₂ are each independently —N(R^{y1})— or —O—, wherein R^{y1} is hydrogen or C₁-C₆ alkyl.

In some embodiments, L is a linker of Formula (IIb) has the structure of:

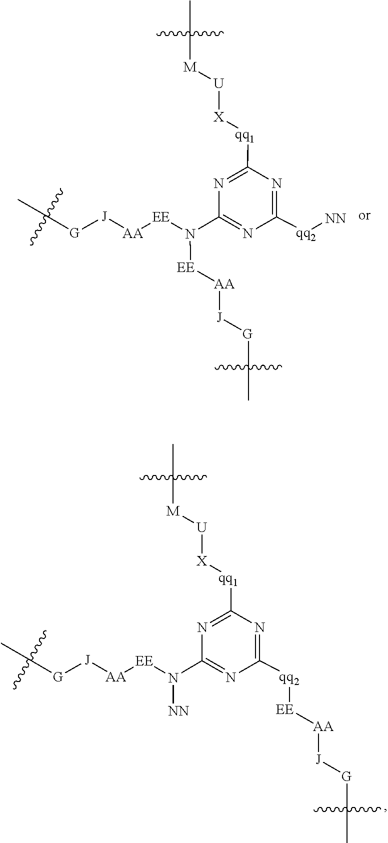

wherein
M is

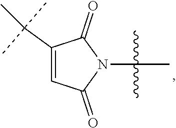

wherein the dashed line indicates the point of covalent attachment to the Ab and the wavy line indicates the point of covalent attachment to the remainder of the structure of the linker;
U is —(CH₂)_{b} (R^{v1})ₛₛ(C═O)ᵤ(NH)ᵥ—, wherein
   subscript b is 1, 2, 3, 4, or 5;
   subscript ss is 0;
   subscript u is 0 or 1; and
   subscript v is 0 or 1; or
   —(CH₂CH₂O)_{w}CH₂CH₂(NH)—, wherein subscript w is an integer ranging from 1 to 16;
X is —(CH₂CH₂O)_{w}CH₂CH₂—(C═O)_{d}—, wherein subscript w is an integer ranging from 0 to 16 and subscript d is 0 or 1;
   qq₁ and qq₂ are each independently —N(R^{y1})— or —O—, wherein R^{y1} is hydrogen or C₁-C₆ alkyl;
   NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
EE is absent or is —(CH₂CH₂O)_{x′}CH₂CH₂C(O)—, wherein subscript x′ is an integer ranging from 0 to 16;
AA is absent or has the structure of:

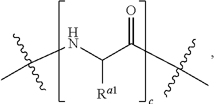

wherein subscript c is an integer ranging from 1 to 12; each R^{a1} is independently —COOH, —NH₂, or an independently selected side chain of an amino acid;
each J is independently absent or has the structure of:

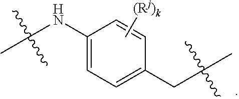

wherein
   each R^{j} is independently halogen, C₁-C₆ alkyl, or —C(O)NH(C₁-C₆ alkyl); and
   subscript k is an integer ranging from 0 to 4;
G is absent or

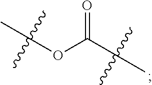 [SMILES: *OC(*)=O]

and wherein the wavy line to M indicates the point of covalent attachment to Ab and the wavy line to G indicates the point of covalent attachment to an independently selected D.

In some embodiments, the wavy line to M of Formula (IIb) indicates the point of covalent attachment to Ab and the wavy line to G of Formula (IIb) indicates the point of covalent attachment to an independently selected D. In some embodiments, the wavy line to G of Formula (IIb) indicates the point of covalent attachment to a secondary alcohol group, a secondary amine group of D, or any combination thereof. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, L is a linker of Formula (III):

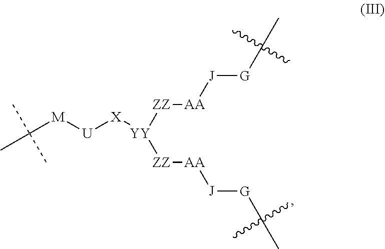

wherein
M is selected from the group consisting of:

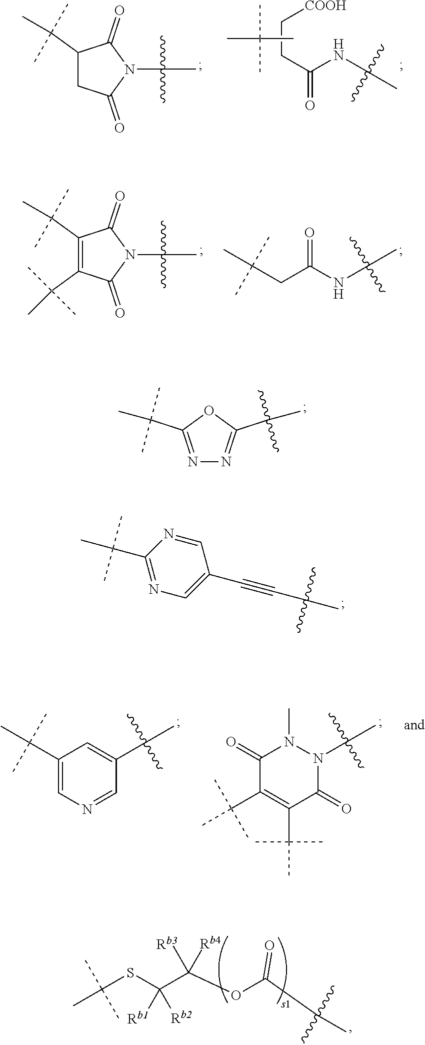

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1,
   wherein the dashed line indicates the point of covalent attachment to the Ab and the wavy line indicates the point of covalent attachment to the remainder of the structure of L;
U is absent or is —(CH₂)_{b} (R^{v1})ₛₛ(C═O)ᵤ(NH)ᵥ—, wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)˜C(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6, and subscript x and subscript y are each independently an integer ranging from 0 to 8;
   —C(O)—C(R^{x3})(R^{x4})—C(O)—, wherein R^{x3} and R^{x4} are each independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—OC(O)—, wherein R^{x5} is hydrogen or C₁-C₆ alkyl, R^{x6} is hydrogen, C₁-C₆ alkyl, or

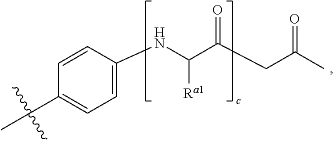

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of:

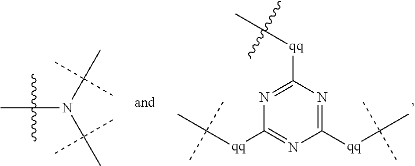

wherein
   each qq is independently —N(R^{y1})— or —O—, wherein R^{y1} is hydrogen or C₁-C₆alkyl; and
wherein
   the wavy line indicates the point of attachment to X, when present, or U, when X is absent, or M, when X and U are absent; and
   each dashed line indicates the point of attachment to ZZ when ZZ is present, or to AA when ZZ is absent, or to J when AA and ZZ are absent, or to G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of:

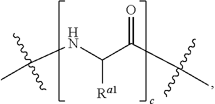

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

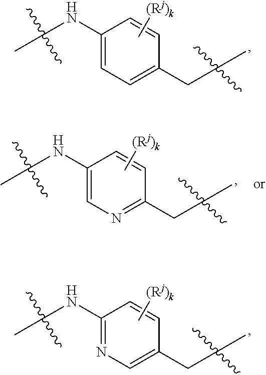

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
each G is independently absent,

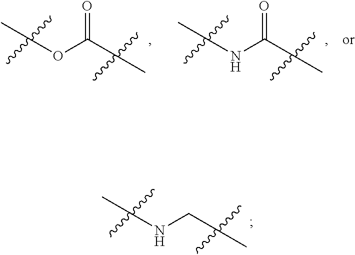

and
wherein the wavy line to M of Formula (III) indicates the point of covalent attachment to Ab and the wavy line to each G of Formula (III) indicates the point of covalent attachment to D,
wherein subscript z is an integer ranging from 1 to 14.

In some embodiments, YY is a branching unit selected from the group consisting of:

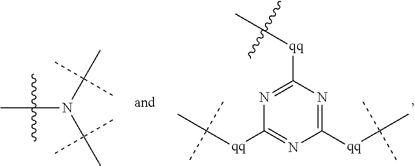

wherein each qq is independently —N(R^{y1})— or —O—, wherein R^{y1} is hydrogen or C₁-C₆ alkyl.

In some embodiments, the branching unit (YY) is

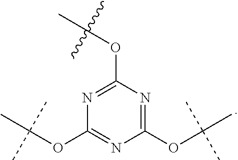

In some embodiments, the branching unit (YY) is

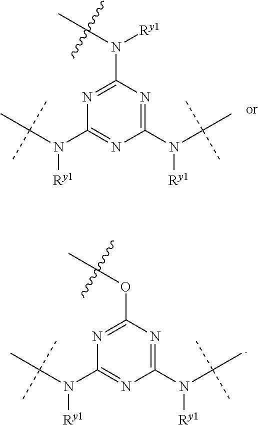

and each R^{y1} is independently hydrogen or C₁-C₆ alkyl. In some embodiments, R^{y1} is hydrogen. In some embodiments, R^{y1} is C₁ alkyl. In some embodiments, R^{y1} is C₂ alkyl. In some embodiments, R^{y1} is C₃ alkyl. In some embodiments, R^{y1} is C₄ alkyl. In some embodiments, R^{y1} is C₅ alkyl. In some embodiments, R^{y1} is C₆ alkyl.

In some embodiments, the branching unit (YY) is

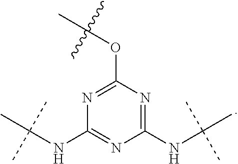

In some embodiments, the branching unit (YY) is an at least tri-substituted carbocycle, an at least tri-substituted heterocycle, a tertiary carbon atom, or a tertiary nitrogen atom. In some embodiments, the branching unit comprises at least three functional groups independently selected from the group consisting of an amine, a carboxylate, a thiocarboxylate, hydroxyl, thiol, carbamate, thiocarbamate, sulfonate, sulfonamide, phosphonate, and phosphinate.

In some embodiments, each ZZ is independently —(CH₂CH₂O)_{w′}CH₂CH₂C(O)—, wherein subscript w′ is an integer ranging from 0 to 16. In some embodiments, ZZ is —(CH₂CH₂O)_{w′}CH₂CH₂C(O)—, wherein w′ is an integer ranging from 0 to 8. In some embodiments, ZZ has the structure of:

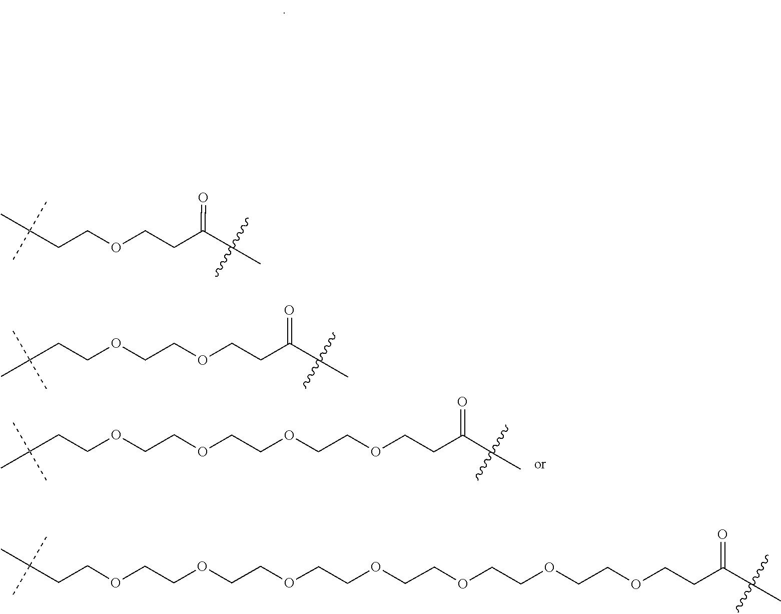

In some embodiments, the linker of Formula (III) has the structure of:

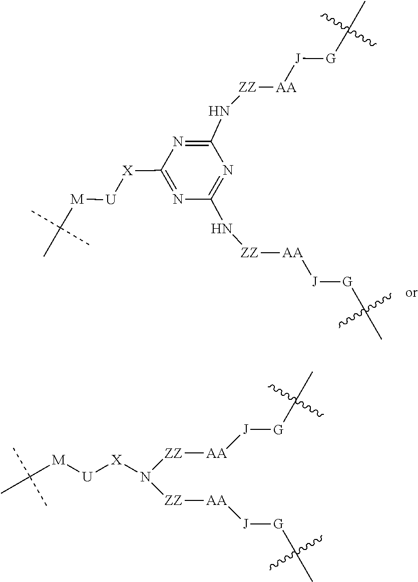

wherein
M is

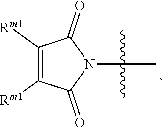

U is absent or is —(CH₂)_{b}(R^{v1})ₛₛ(C═O)ᵤ(NH)ᵥ—, wherein subscript b is 0, 1, 2, 3, 4, or 5; subscript ss is 0 or 1; subscript u is 0 or 1; subscript v is 0 or 1; R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or —(CH₂CH₂O)_{w}CH₂CH₂(NH)—, wherein subscript w is an integer ranging from 1 to 16;
X is absent or is —(CH₂CH₂O)_{w}CH₂CH₂—(C═O)_{d}—, wherein subscript w is an integer ranging from 0 to 16 and subscript d is 0 or 1;
each ZZ is independently —(CH₂CH₂O)_{w′}CH₂CH₂C(O)—, wherein subscript w′ is an integer ranging from 0 to 16;
each AA is independently absent or has the structure of:

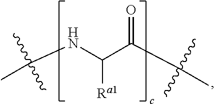

wherein subscript c is an integer ranging from 1 to 12; each R^{a} is independently —COOH, —NH₂, or an independently selected side chain of an amino acid;
each J is independently absent, —(R^{j3})N(C(R^{j1})(R^{j2}))ₘ₁—, or has the structure of:

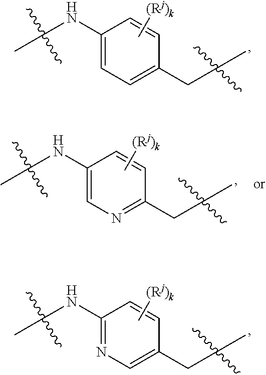

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl); subscript k is an integer ranging from 0 to 4;
each G is independently absent,

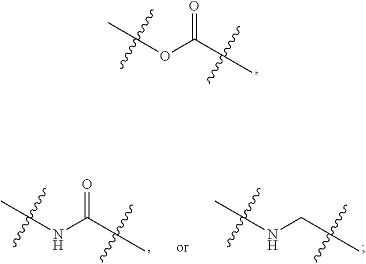

and
wherein the wavy line to M of Formula (III) indicates the point of covalent attachment to Ab and the wavy lined to G of Formula (III) indicates the point of covalent attachment to D. In some embodiments, the wavy lined to G of Formula (III) indicates the point of covalent attachment to a secondary alcohol group or secondary amine group of Formula (IA) of the degrader compound (D).

In some embodiments, the linker has the structure of:

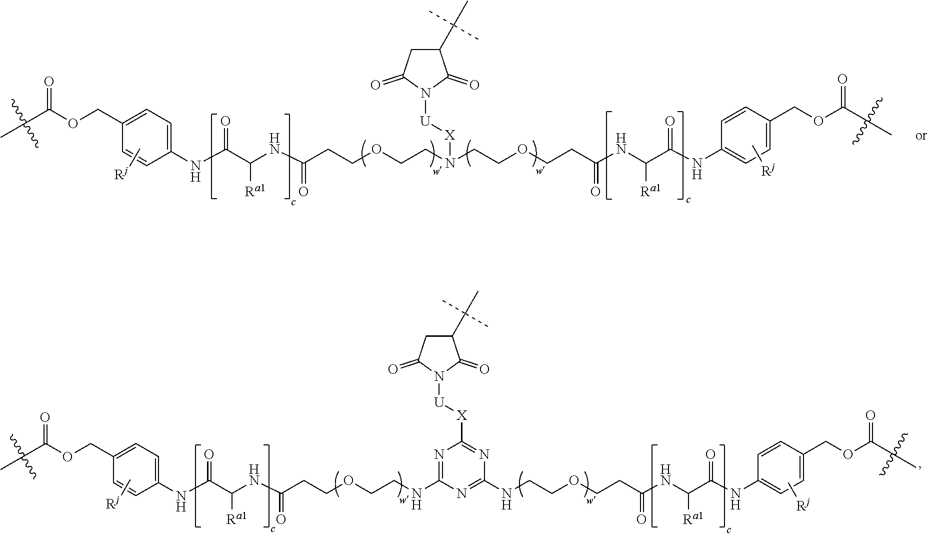

wherein
U is absent or is —(CH₂)_{b}(R^{v1})ₛₛ(C═O)ᵤ(NH)ᵥ—, wherein subscript b is 0, 1, 2, 3, 4, or 5; subscript ss is 0 or 1; subscript u is 0 or 1; subscript v is 0 or 1; R^{V1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or —(CH₂CH₂O)_{w}CH₂CH₂(NH)—, wherein subscript w is an integer ranging from 1 to 16;
X is absent or is —(CH₂CH₂O)_{w}CH₂CH₂—(C═O)_{d}—, wherein subscript w is an integer ranging from 0 to 8 and subscript d is 0 or 1;
subscript c is an integer ranging from 1 to 12 and each R^{a1} is —COOH, —NH₂, or an independently selected side chain of an amino acid, wherein each amino acid is selected from the group consisting of alanine, valine, citrulline, glutamic acid, tryptophan, glycine, phenylalanine, and lysine;
each R^{j} is independently halogen, C₁-C₆ alkyl, or —C(O)NH(C₁-C₆ alkyl); and subscript k is an integer ranging from 0 to 4.

In some embodiments, the linker has the structure of:

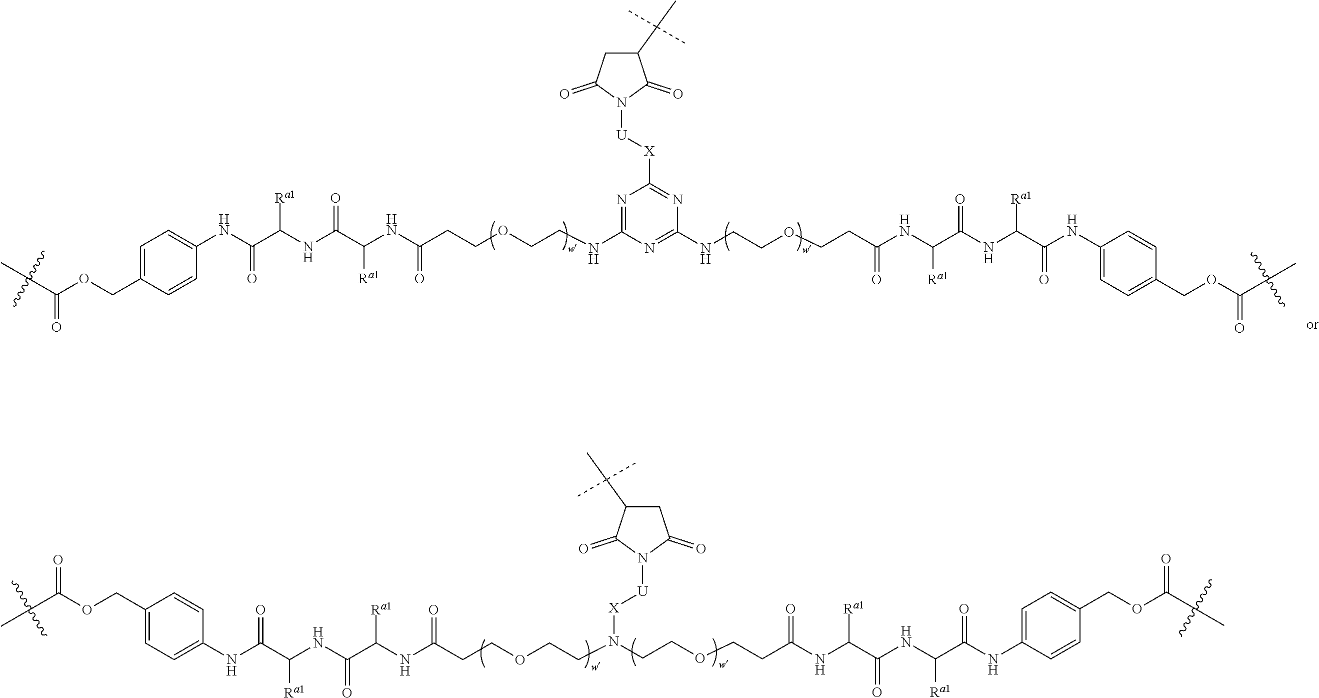

wherein
U is absent or is —(CH₂)_{b}(R^{v1})ₛₛ(C═O)ᵤ(NH)ᵥ—, wherein subscript b is 0, 1, 2, 3, 4, or 5; subscript ss is 0 or 1; subscript u is 0 or 1; subscript v is 0 or 1; R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or —(CH₂CH₂O)_{w}CH₂CH₂(NH)—, wherein subscript w is an integer ranging from 1 to 16;
X is absent or is —(CH₂CH₂O)_{w}CH₂CH₂—(C═O)_{d}—, wherein subscript w is an integer ranging from 0 to 8 and subscript d is 0 or 1;
subscript c is an integer ranging from 1 to 12 and each R^{a1} is —COOH, —NH₂, or an independently selected side chain of an amino acid, wherein each amino acid is selected from the group consisting of alanine, valine, glutamic acid, citrulline, tryptophan, glycine, phenylalanine, and lysine.

In some embodiments, the linker has the structure of:

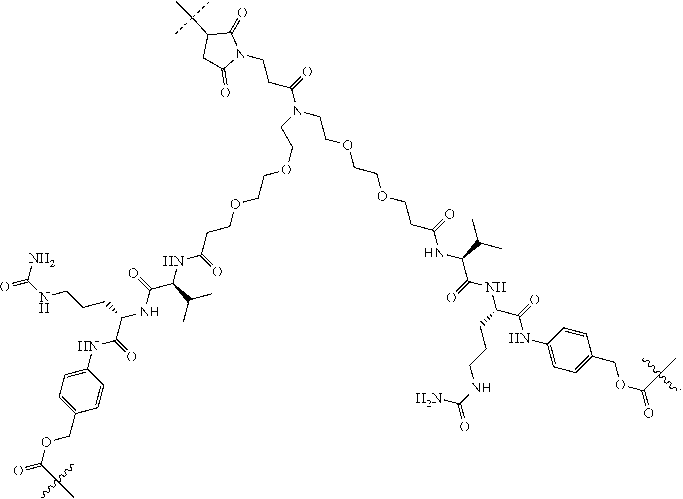

In some embodiments, the linker has the structure of:

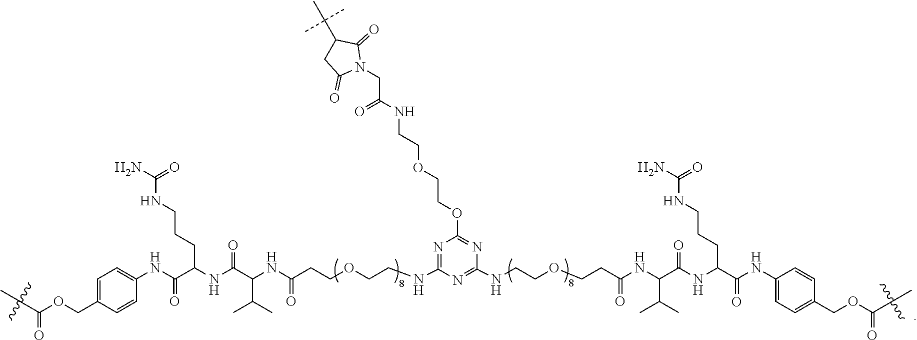

In some embodiments, L is a linker of Formula (III), and each D is independently a degrader compound of Formula (I). In some embodiments, each D independently comprises a small molecule E3 Ubiquitin Ligase binding moiety that binds a Von Hippel-Lindau E3 Ubiquitin Ligase. In some embodiments, each D independently comprises a small molecule E3 Ubiquitin Ligase binding moiety that binds a Cereblon E3 Ubiquitin Ligase. In some embodiments, one D comprises a small molecule E3 Ubiquitin Ligase binding moiety that binds a Von Hippel-Lindau E3 Ubiquitin Ligase, and the other D comprises a small molecule E3 Ubiquitin Ligase binding moiety that binds a Cereblon E3 Ubiquitin Ligase. In some embodiments, each D is the same degrader compound of Formula (I). In some embodiments, each D is a different degrader compound of Formula (I). In some embodiments, each D has a structure of:

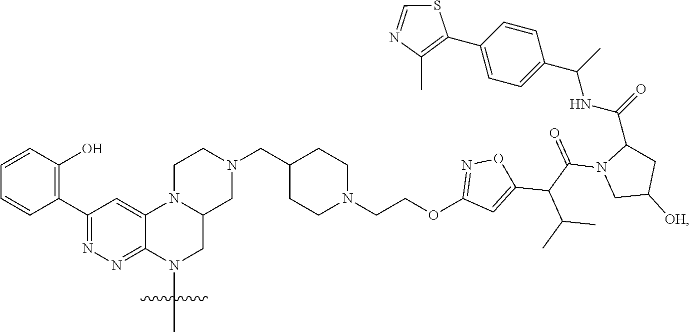 [SMILES: *N1CC2CN(CC3CCN(CCOc4cc(C(C(=O)N5CC(O)CC5C(=O)NC(C)c5ccc(-c6scnc6C)cc5)C(C)C)on4)CC3)CCN2c2cc(-c3ccccc3O)nnc21]

wherein the wavy line indicates the point of covalent attachment to L. In some embodiments, each D has a structure of:

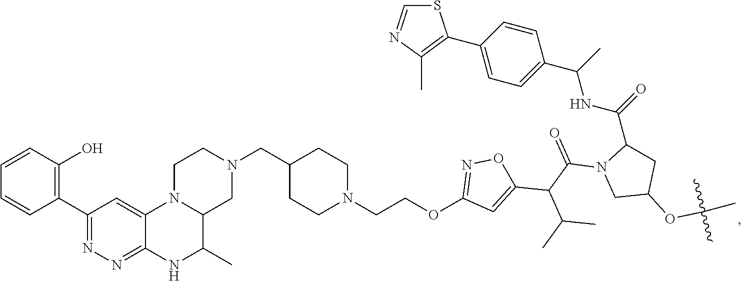 [SMILES: *OC1CC(C(=O)NC(C)c2ccc(-c3scnc3C)cc2)N(C(=O)C(c2cc(OCCN3CCC(CN4CCN5c6cc(-c7ccccc7O)nnc6NC(C)C5C4)CC3)no2)C(C)C)C1]

wherein the wavy line indicates the point of covalent attachment to L. In some embodiments, each D has a structure of:

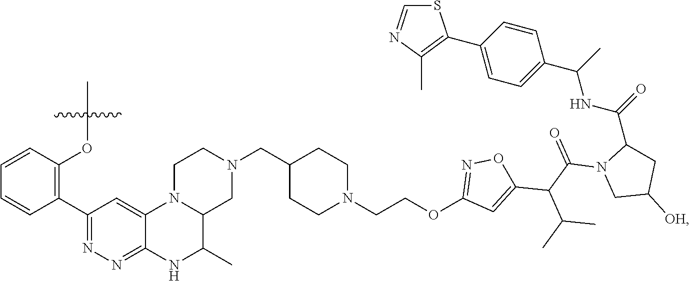 [SMILES: *Oc1ccccc1-c1cc2c(nn1)NC(C)C1CN(CC3CCN(CCOc4cc(C(C(=O)N5CC(O)CC5C(=O)NC(C)c5ccc(-c6scnc6C)cc5)C(C)C)on4)CC3)CCN21]

wherein the wavy line indicates the point of covalent attachment to L. In some embodiments, each D has a structure of

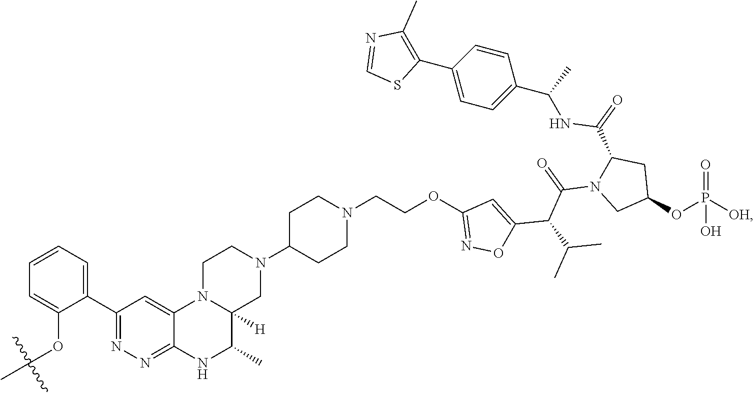 [SMILES: *Oc1ccccc1-c1cc2c(nn1)N[C@@H](C)[C@@H]1CN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)CCN21]

wherein the wavy line indicates the point of covalent attachment to L. In some embodiments, each D has a structure of

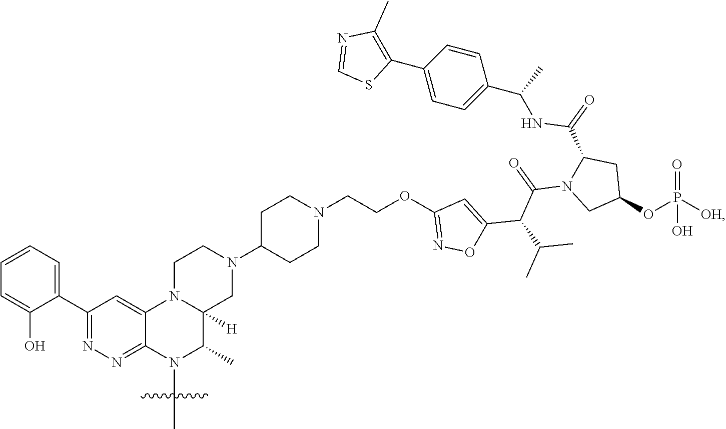 [SMILES: *N1c2nnc(-c3ccccc3O)cc2N2CCN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)C[C@H]2[C@@H]1C]

wherein the wavy line indicates the point of covalent attachment to L. In some embodiments, each D has a structure of

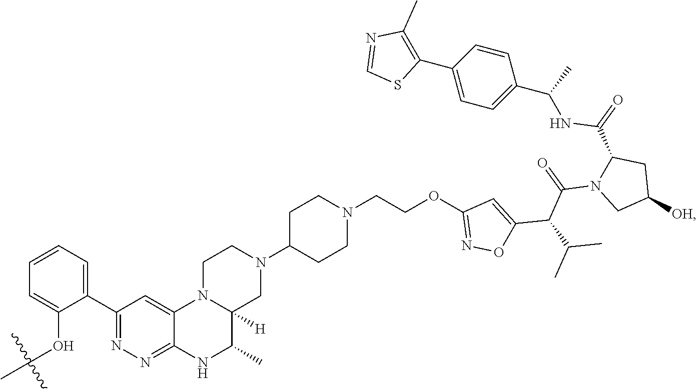 [SMILES: *Oc1ccccc1-c1cc2c(nn1)N[C@@H](C)[C@@H]1CN(C3CCN(CCOc4cc([C@H](C(=O)N5C[C@H](O)C[C@H]5C(=O)N[C@@H](C)c5ccc(-c6scnc6C)cc5)C(C)C)on4)CC3)CCN21]

wherein the wavy line indicates the point of covalent attachment to L. In some embodiments, each D has a structure of

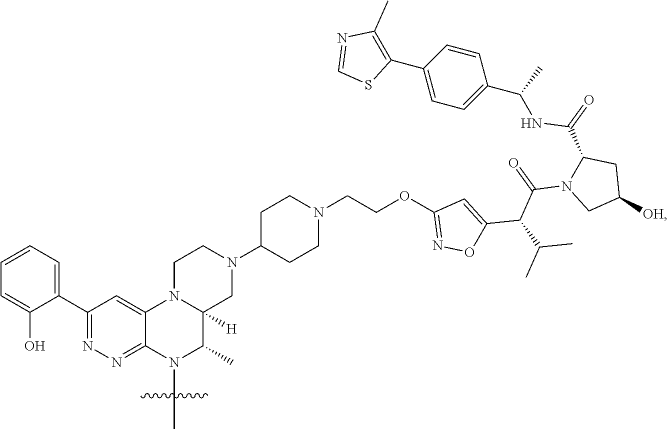 [SMILES: *N1c2nnc(-c3ccccc3O)cc2N2CCN(C3CCN(CCOc4cc([C@H](C(=O)N5C[C@H](O)C[C@H]5C(=O)N[C@@H](C)c5ccc(-c6scnc6C)cc5)C(C)C)on4)CC3)C[C@H]2[C@@H]1C]

wherein the wavy line indicates the point of covalent attachment to L.

### Degrader Compounds (D)

Exemplary degrader compounds that can be used in the antibody-drug conjugates and drug-linker compounds provided herein are described in WO 2021/252666, WO 2022/099117, WO 2023/287787, WO 2023/220577, PCT/US2023/074324, and PCT/US2023/074324, each of which is incorporated herein by reference in its entirety.

In some aspects, provided is an antibody-drug conjugate or a drug-linker compound comprising a degrader compound, wherein the degrader compound is of Formula (I):

PTM-ULM (I)

or a pharmaceutically acceptable salt or solvate thereof, wherein PTM (Protein Targeting Moiety) is a moiety of Formula IA:

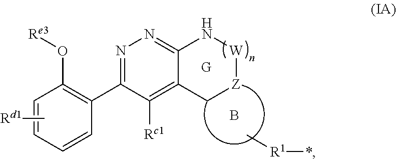

wherein
R¹ is a covalent bond or a chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is H, —C(O)R^{f}, —CH₂—O—P(O)(OR^{g})₂, or —P(O)(OR^{g})₂; wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N, or CR^{h} wherein R^{h}=H, C₁₋₃ alkyl, or absent or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z; and ULM is a small molecule E3 Ubiquitin Ligase binding moiety that binds a Von Hippel-Lindau E3 Ubiquitin Ligase or a Cereblon E3 Ubiquitin Ligase.

### PTM

In some aspects, the degrader compounds of Formula I includes a PTM. According to the disclosure, the PTM in the degrader compounds of Formula I is a moiety of Formula IA

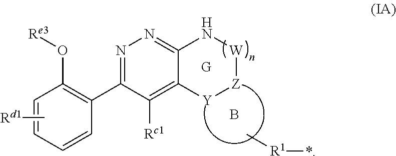

According to the disclosure, B is a ring fused to ring “G” via Y and Z.

In some aspects, B in Formula IA is an optionally substituted 5-7 membered cycloalkyl ring, an optionally substituted 5-7 membered heteroaryl ring, or an optionally substituted 5-7 membered heterocyclic ring. In some embodiments, B in Formula IA is an optionally substituted 5-7 membered cycloalkyl ring. In some embodiments, B in Formula IA is an unsubstituted 5-7 membered cycloalkyl ring. In some embodiments, B is Formula IA is a substituted 5-7 membered cycloalkyl ring wherein the substituents are hydroxy, halogen, alkoxy, alkyl, haloalkyl, amino, alkylamino, or cyano.

In some embodiments, B in Formula IA is an unsubstituted 5-7 membered heteroaryl ring. In some embodiments, B in Formula IA is an unsubstituted 5-7 membered heteroaryl ring. In some embodiments, B in Formula IA is a substituted 5-7 membered heteroaryl ring, wherein substituents are hydroxy, halogen, alkoxy, alkyl, haloalkyl, amino, alkylamino, or cyano.

In other embodiments, B in Formula IA is an unsubstituted 5-7 membered heterocyclic ring. In some embodiments, B in Formula IA is an unsubstituted 5-7 membered heterocyclic ring. In some embodiments, B in Formula IA is a substituted 5-7 membered heterocyclic ring, wherein the substituents are hydroxy, halogen, alkoxy, alkyl, haloalkyl, amino, alkylamino, cyano.

In some aspects, n in Formula IA is 0, 1, 2 or 3. In some embodiments, n=0. In other embodiments, n=1. In other embodiments, n=2. In other embodiments, n=3.

In some aspects, each W in Formula IA is independently optionally substituted —CH₂—, —C(O)—, —S(O)—, or —S(O)₂—, wherein when n=2 or 3, only one W may be —C(O)—, —S(O)—, or —S(O)₂—and the other W are —CH₂— or substituted —CH₂—. Preferred substituents when W is substituted —CH₂— include deuterium, C₁₋₃alkyl, C₁₋₃haloalkyl, and C₁₋₄alkoxyl.

In some embodiments, W in Formula IA is optionally substituted —CH₂—. In other embodiments, W in Formula IA is —CH₂—. Preferred substituents when W is substituted —CH₂— include deuterium, C₁₋₃alkyl, C₁₋₃haloalkyl, and C₁₋₄alkoxyl. In some embodiments, W in Formula IA is —C(O)—. In some embodiments, W in Formula IA is —S(O)—. In some embodiments, W in Formula IA is —S(O)₂—. In embodiments of the disclosure wherein n is 2 or 3, then only one W may be —C(O)—, —S(O)—, or —S(O)₂—and the other W are —CH₂— or substituted —CH₂—. Preferred substituents when W is substituted —CH₂— include deuterium, C₁₋₃alkyl, C₁₋₃haloalkyl, and C₁₋₄alkoxyl.

In some aspects, R^{c1} and R^{d1} in Formula IA are independently H, deuterium, halo, C₁₋₃ alkyl, C₁₋₃ haloalkyl, or C₁₋₄ alkoxyl. In some embodiments, R^{c1} is H. In some embodiments, R^{c1} is deuterium. In some embodiments, R^{c1} is halo, e.g., —F, —Cl, —Br, or —I. In some embodiments, R^{c1} is C₁₋₃ alkyl, e.g., —C₁ alkyl, —C₂ alkyl, —C₃ alkyl, —CH₃, —CH₂CH₃, and the like. In some embodiments, R^{c1} is C₁₋₃ haloalkyl, e.g., —C₁ haloalkyl, —C₂ haloalkyl, —C₃ haloalkyl, —CF₃, —CH₂CF₃, and the like. In some embodiments, R^{c1} is C₁₋₄ alkoxyl, e.g., —C₁ alkoxyl, —C₂ alkoxyl, —C₃ alkoxyl, —C₄ alkoxyl, —OCH₃, —OCH₂CH₃, and the like. In some embodiments, R^{d1} is H. In some embodiments, R^{d1} is deuterium. In some embodiments, R^{d1} is halo, e.g., —F, —Cl, —Br, or —I. In some embodiments, R^{d1} is C₁₋₃ alkyl, e.g., —C₁ alkyl, —C₂ alkyl, —C₃ alkyl, —CH₃, —CH₂CH₃, and the like. In some embodiments, R^{d1} is C₁₋₃ haloalkyl, e.g., —C₁ haloalkyl, —C₂ haloalkyl, —C₃ haloalkyl, —CF₃, —CH₂CF₃, and the like. In some embodiments, R^{d1} is C₁₋₄ alkoxyl, e.g., —C₁ alkoxyl, —C₂ alkoxyl, —C₃ alkoxyl, —C₄ alkoxyl, —OCH₃, —OCH₂CH₃, and the like.

In some aspects, R^{e3} in Formula IA is H, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl. In some embodiments, R^{e3} is H.

In other embodiments, R^{e3} is —C(O)R^{f} wherein R^{f}is H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl. In other embodiments, R^{e3} is —C(O)R^{f} wherein R^{f}is H. In other embodiments, R^{e3} is —C(O)R wherein R^{f}is C₁₋₄ alkyl, e.g., —C₁ alkyl, —C₂ alkyl, —C₃ alkyl, —C₄ alkyl, —CH₃, —CH₂CH₃, and the like. In other embodiments, R^{e3} is —C(O)R^{f}wherein R^{f}is C₁₋₄ substituted alkyl, e.g., —C₁ substituted alkyl, —C₂ substituted alkyl, —C₃ substituted alkyl, and —C₄ substituted alkyl.

In other embodiments, R^{e3} is —C(O)R^{f} wherein R^{f}is C₃₋₈ cyclcoalkyl, e.g., C₃ cyclcoalkyl, C₄ cyclcoalkyl, C₅ cyclcoalkyl, C₆ cyclcoalkyl, C₇ cyclcoalkyl, and C₈ cyclcoalkyl. In other embodiments, R^{e3} is —C(O)R^{f}wherein R^{f}is C₃₋₈ substituted cyclcoalkyl, e.g., C₃ substituted cyclcoalkyl, C₄ substituted cyclcoalkyl, C₅ substituted cyclcoalkyl, C₆ substituted cyclcoalkyl, C₇ substituted cyclcoalkyl, and C₈ substituted cyclcoalkyl.

In other embodiments, R^{e3} is —C(O)R^{f} wherein R^{f}is C₃₋₈ heterocyclcoalkyl, e.g., C₃ heterocyclcoalkyl, C₄ heterocyclcoalkyl, C₅ heterocyclcoalkyl, C₆ heterocyclcoalkyl, C₇ heterocyclcoalkyl, and C₈ heterocyclcoalkyl. In other embodiments, R^{e3} is —C(O)R^{f} wherein R^{f}is C₃₋₈ substituted heterocyclcoalkyl, e.g., C₃ substituted heterocyclcoalkyl, C₄ substituted heterocyclcoalkyl, C₅ substituted heterocyclcoalkyl, C₆ substituted heterocyclcoalkyl, C₇ substituted heterocyclcoalkyl, and C₈ substituted heterocyclcoalkyl.

In other embodiments, R^{e3} is —P(O)(OR^{g})₂; wherein each R^{g} is independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl. In other embodiments, R^{e3} is —P(O)(OR^{g})₂; wherein each R^{g} is H. In other embodiments, R^{e3} is —P(O)(OR^{g})₂; wherein each R^{g} is C₁₋₄ alkyl, e.g., —C₁ alkyl, —C₂ alkyl, —C₃ alkyl, —C₄ alkyl, —CH₃, —CH₂CH₃, and the like. In other embodiments, R^{e3} is —P(O)(OR^{g})₂; wherein one R^{j} is H and the other R^{g} is C₁₋₄ alkyl, e.g., —C₁ alkyl, —C₂ alkyl, —C₃ alkyl, —C₄ alkyl, —CH₃, —CH₂CH₃, and the like. In other embodiments, R^{e3} is —P(O)(OR^{g})₂; wherein at least one R^{g} is C₁₋₄ substituted alkyl, e.g., —C₁ substituted alkyl, —C₂ substituted alkyl, —C₃ substituted alkyl, and —C₄ substituted alkyl. In other embodiments, R^{e3} is —P(O)(OR^{g})₂; wherein at least one R^{g} is C₃₋₈ cyclcoalkyl, e.g., C₃ cyclcoalkyl, C₄ cyclcoalkyl, C₅ cyclcoalkyl, C₆ cyclcoalkyl, C₇ cyclcoalkyl, and C₈ cyclcoalkyl. In other embodiments, R^{e3} is —P(O)(OR^{g})₂; wherein at least one R^{g} is C₃₋₈ substituted cyclcoalkyl, e.g., C₃ substituted cyclcoalkyl, C₄ substituted cyclcoalkyl, C₈ substituted cyclcoalkyl, C₆ substituted cyclcoalkyl, C₇ substituted cyclcoalkyl, and C₈ substituted cyclcoalkyl. In other embodiments, R^{e3} is —P(O)(OR^{g})₂; wherein at least one R^{g} is C₃₋₈ heterocyclcoalkyl, e.g., C₃ heterocyclcoalkyl, C₄ heterocyclcoalkyl, C₅ heterocyclcoalkyl, C₆ heterocyclcoalkyl, C₇ heterocyclcoalkyl, and C₈ heterocyclcoalkyl. In other embodiments, R^{e3} is —P(O)(OR^{g})₂; wherein at least one R^{g} is C₃₋₈ substituted heterocyclcoalkyl, e.g., C₃ substituted heterocyclcoalkyl, C₄ substituted heterocyclcoalkyl, C₅ substituted heterocyclcoalkyl, C₆ substituted heterocyclcoalkyl, C₇ substituted heterocyclcoalkyl, and C₈ substituted heterocyclcoalkyl. In other embodiments, R^{e3} is —CH₂—O—P(O)(OR^{g})₂; wherein each R^{g} is H. In other embodiments, R^{e3} is —CH₂—O—P(O)(OR^{g})₂; wherein each R^{g} is C₁₋₄ alkyl, e.g., —C₁ alkyl, —C₂ alkyl, —C₃ alkyl, —C₄ alkyl, —CH₃, —CH₂CH₃, and the like. In other embodiments, R^{e3} is —CH₂—O—P(O)(OR^{g})₂; wherein one R^{g} is H and the other R^{g} is C₁₋₄ alkyl, e.g., —C₁ alkyl, —C₂ alkyl, —C₃ alkyl, —C₄ alkyl, —CH₃, —CH₂CH₃, and the like. In other embodiments, R^{e3} is —CH₂—O—P(O)(OR^{g})₂; wherein at least one R^{j} is C₁₋₄ substituted alkyl, e.g., —C₁ substituted alkyl, —C₂ substituted alkyl, —C₃ substituted alkyl, and —C₄ substituted alkyl. In other embodiments, R^{e3} is —CH₂—O—P(O)(OR^{g})₂; wherein at least one R^{j} is C₃₋₈ cyclcoalkyl, e.g., C₃ cyclcoalkyl, C₄ cyclcoalkyl, C₅ cyclcoalkyl, C₆ cyclcoalkyl, C₇ cyclcoalkyl, and C₈ cyclcoalkyl. In other embodiments, R^{e3} is —CH₂—O—P(O)(OR^{g})₂; wherein at least one R^{g} is C₃₋₈ substituted cyclcoalkyl, e.g., C₃ substituted cyclcoalkyl, C₄ substituted cyclcoalkyl, C₅ substituted cyclcoalkyl, C₆ substituted cyclcoalkyl, C₇ substituted cyclcoalkyl, and C₈ substituted cyclcoalkyl. In other embodiments, R^{e3} is —CH₂—O—P(O)(OR^{g})₂; wherein at least one R^{g} is C₃₋₈ heterocyclcoalkyl, e.g., C₃ heterocyclcoalkyl, C₄ heterocyclcoalkyl, C₅ heterocyclcoalkyl, C₆ heterocyclcoalkyl, C₇ heterocyclcoalkyl, and C₈ heterocyclcoalkyl. In other embodiments, R^{e3} is —CH₂—O—P(O)(OR^{g})₂; wherein at least one R^{g} is C₃₋₈ substituted heterocyclcoalkyl, e.g., C₃ substituted heterocyclcoalkyl, C₄ substituted heterocyclcoalkyl, C₅ substituted heterocyclcoalkyl, C₆ substituted heterocyclcoalkyl, C₇ substituted heterocyclcoalkyl, and C₈ substituted heterocyclcoalkyl.

In some aspects, Z and Y in Formula IA are each independently N or CR^{h}, wherein R^{h}=H or C₁₋₃ alkyl or may be absent when n=1-3 such that a double bond is formed between Z and Y, or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl. Examples of these embodiments include:

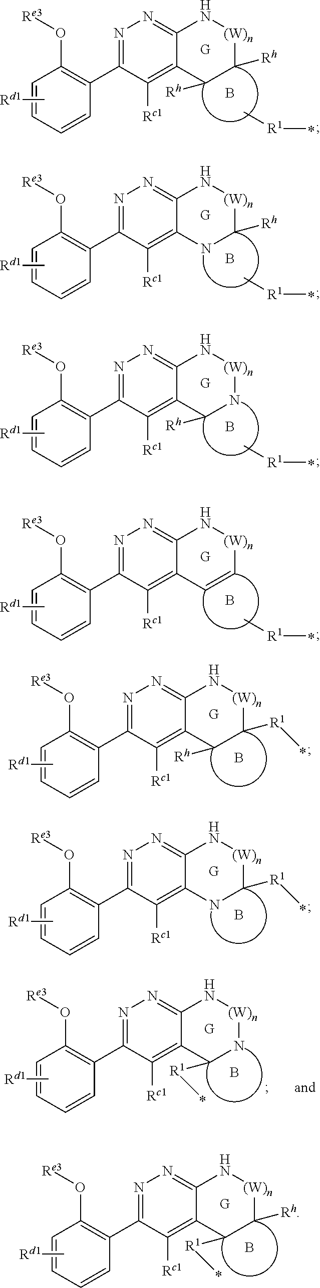

In some embodiments, Z is N. In other embodiments, Z is CR^{h} wherein R^{h}=H or C₁₋₃ alkyl. In other embodiments, Z is CR^{h} wherein R^{h}=H. In other embodiments, Z is CR^{h} wherein R^{h}=H or C₁₋₃ alkyl. In other embodiments, Z is CR^{h} wherein R^{h}=methyl. In other embodiments, Z is CR^{h} wherein R^{h}=ethyl. In other embodiments, Z is CR^{h} wherein R^{h}=propyl.

In other embodiments, Z is CR^{h}wherein R^{h}=absent, and Z is bonded to Y by a double bond. In some embodiments, Z is C and is attached to R¹.

In some embodiments, Y is N. In other embodiments, Y is CR^{h} wherein R^{h}=H or C₁₋₃ alkyl. In other embodiments, Y is CR^{h} wherein R^{h}=absent, and Y is bonded to Z by a double bond. In some embodiments, Y is C and is attached to R¹. In other embodiments, Y is CR^{h}wherein R^{h}=H. In other embodiments, Y is CR^{h}wherein R^{h}=H or C₁₋₃ alkyl. In other embodiments, Y is CR^{h} wherein R^{h}=methyl. In other embodiments, Y is CR^{h} wherein R^{h}=ethyl. In other embodiments, Y is CR^{h} wherein R^{h}=propyl.

In some embodiments, the PTM is a moiety of Formula IA wherein * is a point of attachment to ULM. In some aspects, R¹ in Formula IA is a covalent bond, or chemical moiety that links PTM and ULM.

In some embodiments, R¹ in Formula IA is a covalent bond. In other embodiments, R¹ in Formula IA is a chemical moiety that links PTM and ULM.

Chemical moieties that are used to link PTM and ULM moieties are known in the art. In some embodiments, R^{j} in Formula IA is a chemical moiety that is used to link a PTM and ULM that is known in the art.

In some embodiments, R^{j} in Formula IA is a chemical moiety that is used to link a PTM and ULM as described in U.S. Patent Application Publication No. 2019/0300521, the entirety of which is incorporated by reference herein. In other embodiments, R^{j} in Formula IA is a chemical moiety that is used to link a PTM and ULM as described in U.S. Patent Application Publication No. 2019/0255066, the entirety of which is incorporated by reference herein. In other embodiments, R^{j} in Formula IA is a chemical moiety that is used to link a PTM and ULM as described in WO 2019/084030, the entirety of which is incorporated by reference herein. In other embodiments, R^{j} in Formula IA is a chemical moiety that is used to link a PTM and ULM as described in WO 2019/084026, the entirety of which is incorporated by reference herein.

In some embodiments, R^{j} in Formula IA is a chemical structural unit represented by the formula:

-(A)_{q}-,

wherein:
q is an integer from 1 to 14;
each A is independently selected from the group consisting of a bond, CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b} groups, 3-11 membered heteocyclyl optionally substituted with 0-6 R^{1a} and/or R^{1b} groups, aryl optionally substituted with 0-6 R^{1a} and/or R^{1b} groups, and heteroaryl optionally substituted with 0-6 R^{1a} and/or R^{1b} groups,
and R^{1a}, R^{1b}, R^{1c}, R^{1d} and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups.

In these embodiments, q represents the number of connected A groups. For example, when q=1, -(A)_{q}- is -A₁-; when q=2, -(A)_{q}- is -A₁-A₂-; when q=3, -(A)_{q}- is -A₁-A₂-A₃-; when q=4, -(A)_{q}- is -A1-A₂-A₃-A₄-; when q=5, -(A)_{q}- is -A1-A₂-A₃-A₄-A₅-; when q=6, -(A)_{q}- is -A1-A₂-A₃-A₄-A₅-A₆-; when q=7, -(A)_{q}- is -A1-A₂-A₃-A₄-A₅-A₆-A₇-; when q=8, -(A)_{q}- is -A₁-A₂-A₃-A₄-A₅-A₆-A₇-A₈-; when q=9, -(A)_{q}- is -A1-A₂-A₃-A₄-A₅-A₆-A₇-A₈-A₉-; when q=10, -(A)_{q}- is -A1-A₂-A₃-A₄-A₅-A₆-A₇-A₈-A₉-A₁₀-; when q=11, -(A)_{q}- is -A₁-A₂-A₃-A₄-A₅-A₆-A₇-A₈-A₉-A₁₀-A₁₁-; when q=12, -(A)_{q}- is -A₁-A₂-A₃-A₄-A₅-A₆-A₇-A₈-A₉-A₁₀-A₁₁-A₁₂-; when q=13, -(A)_{q}- is -A₁-A₂-A₃-A₄-A₅-A₆-A₇-A₈-A₉-A₁₀-A₁₁-A₁₂-A₁₃-; and when q=14, -(A)_{q}- is -A₁-A₂-A₃-A₄-A₅-A₆-A₇-A₈-A₉-A₁₀-A₁₁-A₁₂-A₁₃-A₁₄-.

In some embodiments, q=5 and R¹ is a chemical moiety represented by the formula: -A₁-A₂-A₃-A₄-A₅-, wherein each of A₁, A₃ and A₅ is independently selected from the group consisting of a bond, —(CR^{1a}R^{1b})₀₋₄O(CR^{1a}R^{1b})₀₋₄, —(CR^{1a}R^{1b})₀₋₄S(CR^{1a}R^{1b})₀₋₄, —(CR^{1a}R^{1b})₀₋₄NR^{1c}(CR^{1a}R^{1b})₀₋₄, —(CR^{1a}R^{1b})₀₋₄SO(CR^{1a}R^{1b})₀₋₄, —(CR^{1a}R^{1b})₀₋₄SO₂(CR^{1a}R^{1b})₀₋₄, —(CR^{1a}R^{1b})₀₋₄ SO₂NR^{1c}(CR^{1a}R^{1b})₀₋₄, —(CR^{1a}R^{1b})₀₋₄SONR^{1c}(CR^{1a}R^{1b})₀₋₄, —(CR^{1a}R^{1b})₀₋₄SO(═NR^{1c})(CR^{1a}R^{1b})₀₋₄, —(CR^{1a}R^{1b})₀₋₄ SO(═NR^{1c})NR^{1d}(CR^{1a}R^{1b})₀₋₄, —(CR^{1a}R^{1b})₀₋₄CONR^{1c}(CR^{1a}R^{1b})₀₋₄, —(CR^{1a}R^{1b})₀₋₄C(O)O(CR^{1a}R^{1b})₀₋₄, —(CR^{1a}R^{1b})₀₋₄NR^{1c}CONR^{1d}(CR^{1a}R^{1b})₀₋₄, —(CR^{1a}R^{1b})₀₋₄NR^{1c}C(O)O(CR^{1a}R^{1b})₀₋₄, —(CR^{1a}R^{1b})₀₋₄NR^{1c}SO₂NR^{1d}(CR^{1a}R^{1b})₀₋₄, —(CR^{1a}R^{1b})₀₋₄C(O)(CR^{1a}R^{1b})₀₋₄, —(CR^{1a}R^{1b})₀₋₄CR^{1a}═CR^{1b}(CR^{1a}R^{1b})₀₋₄, —(CR^{1a}R^{1b})₀₋₄C≡C(CR^{1a}R^{1b})₀₋₄, —(CR^{1a}R^{1b})₀₋₄SiR^{1a}R^{1b}(CR^{1a}R^{1b})₀₋₄, —(CR^{1a}R^{1b})₀₋₄P(O)R^{1a}(CR^{1a}R^{1b})₀₋₄, —(CR^{1a}R^{1b})₀₋₄P(O)OR^{1a}(CR^{1a}R^{1b})₀₋₄, (CR^{1a}R^{1b})₁₋₄, optionally substituted 3-11 membered cycloalkyl, 3-11 membered heterocyclyl, aryl, and heteroaryl; wherein each of A₂ and A₄ is independently selected from the group consisting of is independently selected from the group consisting of a bond, (CR^{1a}R^{1b})₁₋₄, optionally substituted 3-11 membered cycloalkyl, 3-11 membered heterocyclyl, aryl, and heteroaryl; wherein R^{1a} and R^{1b} are each independently selected from the group consisting of —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC1-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, and —NHSO₂NH₂; and R^{1c} and R^{1d} are each independently selected from the group consisting of H, deuterium, optionally substituted C₁₋₄ alkyl, C₃₋₈ cyclcoalkyl, C₃₋₈ heterocyclcoalkyl, aryl, and heteroaryl.

In some embodiments, q=4 and R¹ is a chemical moiety represented by the formula: -A₁-A₂-A₃-A₄-, wherein each of A₁₋₄ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl; wherein R^{1a} and R^{1b} are each independently selected from the group consisting of —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and R^{1c} and R^{1d} are each independently selected from the group consisting of H, deuterium, optionally substituted C₁₋₄ alkyl, C₃₋₈ cyclcoalkyl, C₃₋₈ heterocyclcoalkyl, aryl, and heteroaryl.

In other embodiments, q=3 and R¹ is a chemical moiety represented by the formula: -A₁-A₂-A₃-, wherein each of A₁₋₃ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl; wherein R^{1a} and R^{1b} are each independently selected from the group consisting of —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, and —NHSO₂NH₂; and R^{1c} and R^{1d} are each independently selected from the group consisting of H, deuterium, optionally substituted C₁₋₄ alkyl, C₃₋₈ cyclcoalkyl, C₃₋₈ heterocyclcoalkyl, aryl, and heteroaryl.

In other embodiments, q=2 and R¹ is a chemical moiety represented by the formula: -A₁-A₂-, wherein each of A₁₋₂ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d} NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl; wherein R^{1a} and R^{1b} are each independently selected from the group consisting of —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC1-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, and —NHSO₂NH₂; and R^{1c} and R^{1d} are each independently selected from the group consisting of H, deuterium, optionally substituted C₁₋₄ alkyl, C₃₋₈ cyclcoalkyl, C₃₋₈ heterocyclcoalkyl, aryl, and heteroaryl.

In other embodiments, q=1 and R^{j} is a chemical moiety represented by the formula: -A₁, wherein A₁ is selected from the group consisting of O, S, SO, SO₂, NR^{1c}SO₂NR^{1c}SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl; wherein R^{1a} and R^{1b} are each independently selected from the group consisting of —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, and —NHSO₂NH₂; and R^{1c} and R^{1d} are each independently selected from the group consisting of H, deuterium, optionally substituted C₁₋₄ alkyl, C₃₋₈ cyclcoalkyl, C₃₋₈ hetero-cyclcoalkyl, aryl, and heteroaryl.

In some embodiments, R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}—(CR^{1a}R^{1b})₁₋₅-A-(CR^{1a}R^{1b})₁₋₅— wherein A is O, S, or NR^{1c}—(CR^{1a}R^{1b})₁₋₅-A-(CR^{1a}R^{1b})₁₋₅-A- wherein A is O, S, or NR^{1c}—(CR^{1a}R^{1b})₁₋₅—(CR^{1a}═CR^{1b})—(CR^{1a}R^{1b})₁₋₅—, —(CR^{1a}R^{1b})₁₋₅₋(CR^{1a}═CR^{1b})—(CR^{1a}R^{1b})₁₋₅-A- wherein A is O, S, or NR^{1c}—(CR^{1a}R^{1b})₁₋₅, —(C≡C)—(CR^{1a}R^{1b})₁₋₅—, —(CR^{1a}R^{1b})₁₋₅—(C≡C—(CR^{1a}R^{1b})₁₋₅-A- wherein A is O, S, or NR^{1c}, —(C≡C—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b} groups)-, -(3-11 membered cycloalkyl optionally substituted with 0-6 R^{1a} and/or R^{1b} groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b}groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-(3-11 membered heterocyclyl optionally substituted with 0-6 R^{1a} and/or R^{1b} groups)-A- wherein A is O, S, or NR^{1c}—(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(CR^{1a}R^{1b})₁₋₅-A- wherein A is O, S, or NR^{1c}—(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}—(CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}—(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b} groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}, -(3-11 membered cycloalkyl optionally substituted with 0-6 R^{1a} and/or R^{1b} groups)-(CR^{1a}R^{1b})₁₋₅—, or -(3-11 membered heterocyclyl optionally substituted with 0-6 R^{1a} and/or R^{1b} groups)-(CR^{1a}R^{1b})₁₋₅.

In some embodiments, R¹ is —CR^{1a}═CR^{1b}—, such as, for example, —CH═CH—. In some embodiments, R¹ is —(CR^{1a}R^{1b})₁₋₅, for example —(CH₂)₁₋₅—, —CH₂—, —CH₂CH₂CH₂—and the like. In some embodiments, R¹ is —(CR^{1a}R^{1b})₁₋₅-A- wherein A is O, S, or NR^{1c}, such as for example, —(CH₂)₁₋₅—O—, —(CH₂)₁₋₅—S—, —(CH₂)₁₋₅—NH—, or —(CH₂)₀₋₂—(C(CH₃)₂)—(CH₂)₀₋₂—O—. In other embodiments, R¹ is —(CR^{1a}R^{1b})₁₋₅-A-(CR^{1a}R^{1b})₁₋₅— wherein A is O, S, or NR^{1c}, such as, for example, —(CH₂)₁₋₅—O—(CH₂)₁₋₅—, —(CH₂)₁₋₅—S—(CH₂)₁₋₅—, —(CH₂)₁₋₅—NH—(CH₂)₁₋₅—. In some embodiments, R¹ is —(C≡C)—(CR^{1a}R^{1b})₁₋₅, such as, for example, —(C≡C)—(CH₂)₂—, and the like. In some embodiments, R¹ is —(CR^{1a}R^{1b})₁₋₅-(3-11 membered cycloalkyl optionally substituted with 0-6 R^{1a} and/or R^{1b} groups)-, such as, for example, —CH₂-cyclobutyl-. In some embodiments, R¹ is —(CR^{1a}R^{1b})₁₋₅-(3-11 membered cycloalkyl optionally substituted with 0-6 R^{1a} and/or R^{1b} groups)-(CR^{1a}R^{1b})₁₋₅, such as, for example, —CH₂-cyclobutyl-CH₂—and the like. In some embodiments, R¹ is —(CR^{1a}R^{1b})₁₋₅-(3-11 membered heterocyclyl optionally substituted with 0-6 R^{1a} and/or R^{1b}groups)-(CR^{1a}R^{1b})₁₋₅, such as, for example, —CH₂-azetidinyl-CH₂—. In some embodiments, R¹ is —(CR^{1a}R^{1b})₁₋₅-(3-11 membered heterocyclyl optionally substituted with 0-6 R^{1a} and/or R^{1b}groups)-, such as, for example, —CH₂-azetidinyl-. In some embodiments, R¹ is -(3-11 membered heterocyclyl optionally substituted with 0-6 R^{1a} and/or R^{1b} groups)-(CR^{1a}R^{1b})₁₋₅—, such as, for example, -azetidinyl-CH₂—, -pyrolidnyl-CH₂—, -piperidinyl-CH₂—, and the like. In some embodiments, R¹ is —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, such as, for example, —CH₂— cyclopropyl-CH₂—O—, and the like. In some embodiments, R¹ is —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, such as, for example, —CH₂-piperidinyl-CH₂CH₂—O—, and the like. In some embodiments, R¹ is —(CR^{1a}R^{1b})₁₋₅-(3-11 membered heterocyclyl optionally substituted with 0-6 R^{1a} and/or R^{1b} groups)-A- wherein A is O, S, or NR^{1c}, such as, for example, —CH₂-azetidinyl-O—, and the like. In some embodiments, R¹ is —(CR^{1a}R^{1b})₁₋₅-A-(3-11 membered heterocyclyl optionally substituted with 0-6 R^{1a} and/or R^{1b} groups)- wherein A is O, S, or NR, such as, for example, —CH₂—O-azetidinyl-, —CH₂—NH-azetidinyl-, and the like. In other embodiments, R^{j} is —(CR^{1a}R^{1b})₁₋₅-A-(3-11 membered cycloalkyl optionally substituted with 0-6 R^{1a} and/or R^{1b}groups)- wherein A is O, S, or NR, such as —CH₂—O-cyclobutylene-, —CH₂—NH-cyclobutylene-, and the like. In some embodiments, R^{j} is —(CR^{1a}R^{1b})₁₋₅-A-(CR^{1a}R^{1b})₁₋₅-A- wherein A is O, S, or NR^{1c} such as, for example, —CH₂—O—CH₂CH₂—O—.

In some aspects, the Y in the degrader compound of Formula IA is CR^{h} wherein R^{h} is H or C₁₋₃ alkyl, and the degrader compound of Formula IA has Formula IA-1:

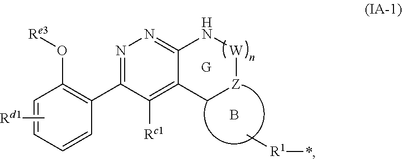

wherein R^{c1}, R^{d1}, R^{e3}, W, Z, B, n, and R¹ are as described above for Formula IA.

In some embodiments, n in Formula IA-1 is 1.

In some embodiments of the degrader compound of Formula IA-1, at least one W is optionally substituted —CH₂—. In some embodiments of the degrader compound of Formula IA-1, at least one W is —CH₂— or substituted —CH₂— wherein the substituents are alkyl, alkoxy, alkylamino. In some embodiments of the degrader compound of Formula IA-1, at least one W is —CH₂—. In some embodiments of the degrader compound of Formula IA-1, one W is —C(O)—. In some embodiments of the degrader compound of Formula IA-1, one W is —S(O)—. In some embodiments of the degrader compound of Formula IA-1, one W is —S(O)₂—.

In some embodiments, B in Formula IA-1 is an optionally substituted 5-7 membered cycloalkyl ring. In some embodiments, B in Formula IA-1 is an optionally substituted 5-7 membered cycloalkyl ring wherein the optional substituents are hydroxy, halogen, alkoxy, alkyl, haloalkyl, amino, alkylamino, or cyano. In other embodiments, B in Formula IA-1 is an optionally substituted 5-7 membered heterocyclic ring. In some embodiments, B in Formula IA-1 is an optionally substituted 5-7 membered heterocyclic ring wherein the optional substituents are hydroxy, halogen, alkoxy, alkyl, haloalkyl, amino, alkylamino, cyano.

In other aspects, the Y in the degrader compound of Formula IA is N, and Z is CR^{h}wherein R^{h} is H or C₁₋₃ alkyl, and the degrader compound of Formula IA has Formula IA-2:

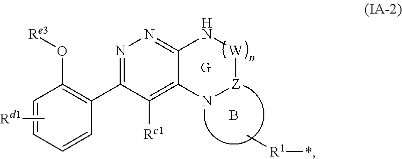

wherein R^{c1}, R^{d1}, R^{e3}, W, B, n, and R¹ are as described above for Formula IA.

In some embodiments, n in Formula IA-2 is 1.

In some embodiments of the degrader compound of Formula IA-2, at least one W is —CH₂— or substituted —CH₂—. In some embodiments of the degrader compound of Formula IA-2, at least one W is —CH₂— or substituted —CH₂— wherein the substituents are alkyl, alkoxy, alkylamino.

In some embodiments of the degrader compound of Formula IA-2, at least one W is —CH₂—. In some embodiments of the degrader compound of Formula IA-2, one W is —C(O)—. In some embodiments of the degrader compound of Formula IA-2, one W is —S(O)—. In some embodiments of the degrader compound of Formula IA-2, one W is —S(O)₂—.

In some embodiments, B in Formula IA-2 is an optionally substituted 5-7 membered heterocyclic ring. In some embodiments, B in Formula IA-2 is an optionally substituted 5-7 membered heterocyclic ring wherein the optional substituents are hydroxy, halogen, alkoxy, alkyl, haloalkyl, amino, alkylamino, cyano. In other embodiments, B in Formula IA-2 is an optionally substituted 5-7 membered heterocyclic ring. In some embodiments, B in Formula IA-2 is an optionally substituted 5-7 membered heterocyclic ring wherein the optional substituents are hydroxy, halogen, alkoxy, alkyl, haloalkyl, amino, alkylamino, or cyano.

In some aspects, the degrader compound of Formula IA is a degrader compound of Formula IA-3:

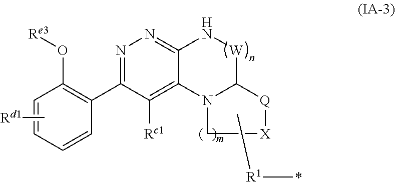

wherein m=1 to 3;
X is optionally substituted —CH₂—, or NH; or, if R¹ is attached to X, then X is —CH— or N;
Q is optionally substituted —CH₂—, optionally substituted —(CH₂)₂—, —C(O)—, optionally substituted —CH₂C(O)—, —S(O)—, —S(O)₂—, optionally substituted —CH₂S(O)₂—, or optionally substituted —CH₂S(O)—; and wherein R^{c1}, R^{d1}, R^{e3}, W, Z, B, n, and R¹ are as described above for Formula IA.

In some embodiments of the degrader compound of Formula IA-3, n=1. In other embodiments of the degrader compound of Formula IA-3, n=2. In other embodiments of the degrader compound of Formula IA-3, n=3.

In some embodiments of the degrader compound of Formula IA-3, X is —CH—. In other embodiments of the degrader compound of Formula IA-3, X is NH. In some of those embodiments of the degrader compound of Formula IA-3 wherein R¹ is attached to X, then X is CH. In other of those embodiments of the degrader compound of Formula IA-3 wherein R¹ is attached to X, then X is N.

In some embodiments of the degrader compound of Formula IA-3, Q is optionally substituted —CH₂—. In some embodiments of the degrader compound of Formula IA-3, Q is optionally substituted —CH₂— wherein the optional substituents are alkyl, alkoxy, or alkylamino. In some embodiments of the degrader compound of Formula IA-3, Q is optionally substituted —(CH₂)₂—. In some embodiments of the degrader compound of Formula IA-3, Q is optionally substituted —(CH₂)₂— wherein the optional substituents are alkyl, alkoxy, or alkylamino. In some embodiments of the degrader compound of Formula IA-3, Q is —C(O)—. In some embodiments of the degrader compound of Formula IA-3, Q is optionally substituted —CH₂C(O)—. In some embodiments of the degrader compound of Formula IA-3, Q is —S(O)—. In some embodiments of the degrader compound of Formula IA-3, Q is —S(O)₂—. In some embodiments of the degrader compound of Formula IA-3, Q is optionally substituted —CH₂S(O)₂—. In some embodiments of the degrader compound of Formula IA-3, Q is optionally substituted —CH₂S(O)—.

In some aspects, the degrader compound of Formula IA is a degrader compound of Formula IA-4:

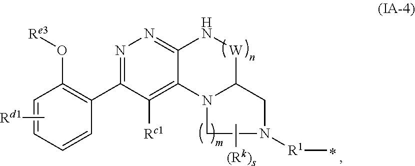

wherein R^{k}=H, deuterium, F, C₁₋₃ alkyl, C₁₋₃ haloalkyl, C₁₋₄ alkoxyl, substituted C₁₋₃ alkyl, substituted C₁₋₃ haloalkyl, or substituted C₁₋₄ alkoxyl; s=0-7; m=1-3; and wherein R^{c1}, R^{d1}, R^{e3}, W, n, and R¹ are as described above for Formula IA.

In some embodiments of the degrader compound of Formula IA-4, n=1. In other embodiments of the degrader compound of Formula IA-4, n=2. In other embodiments of the degrader compound of Formula IA-4, n=3.

In some embodiments of the degrader compound of Formula IA-4, m=1. In other embodiments of the degrader compound of Formula IA-4, m=2. In other embodiments of the degrader compound of Formula IA-4, m=3.

In some embodiments of the degrader compound of Formula IA-4, s=0. In some embodiments of the degrader compound of Formula IA-4, s=1. In other embodiments of the degrader compound of Formula IA-4, s=2. In other embodiments of the degrader compound of Formula IA-4, s=3.

In some embodiments of the degrader compound of Formula IA-4, R^{k}=H. In some embodiments of the degrader compound of Formula IA-4, R^{k}=deuterium. In some embodiments of the degrader compound of Formula IA-4, R^{k}=F. In some embodiments of the degrader compound of Formula IA-4, R^{k}═C₁₋₃ alkyl, for example, C₁ alkyl, C₂ alkyl, C₃ alkyl, —CH₃, —CH₂CH₃, and the like. In some embodiments of the degrader compound of Formula IA-4, R^{k}═C₁₋₃ haloalkyl, for example, C₁ haloalkyl, C₂ haloalkyl, C₃ haloalkyl, —CF₃, —CH₂CF₃, and the like. In some embodiments of the degrader compound of Formula IA-4, R^{k}═C₁₋₄ alkoxyl, for example, C₁ alkoxyl, C₂ alkoxyl, C₃ alkoxyl, —OCH₃, —OCH₂CH₃, and the like. In some embodiments of the degrader compound of Formula IA-4, R^{k}=substituted C₁₋₃ alkyl, for example, substituted C₁ alkyl, substituted C₂ alkyl, substituted C₃ alkyl, and the like. In some embodiments of the degrader compound of Formula IA-4, R^{k}=substituted C₁₋₃ haloalkyl, for example, substituted C₁ haloalkyl, substituted C₂ haloalkyl, substituted C₃ haloalkyl, and the like.

In some embodiments of the degrader compound of Formula IA-4, R^{k}=substituted C₁₋₄ alkoxyl, for example, substituted C₁ alkoxyl, substituted C₂ alkoxyl, substituted C₃ alkoxyl, and the like.

In some aspects, the degrader compound of Formula IA is a degrader compound of Formula IA-5:

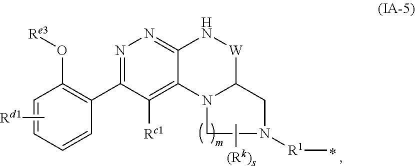

wherein R^{k}=H, deuterium, F, C₁₋₃ alkyl, C₁₋₃ haloalkyl, or C₁₋₄ alkoxyl; m=1-3; and s=0-3, and wherein R^{c1}, R^{d1}, R^{e3}, W, and R¹ are as described above for Formula IA.

In some embodiments of the degrader compound of Formula IA-5, m=1. In other embodiments of the degrader compound of Formula IA-5, m=2. In other embodiments of the degrader compound of Formula IA-5, m=3.

In some embodiments of the degrader compound of Formula IA-5, s=0. In some embodiments of the degrader compound of Formula IA-5, s=1. In other embodiments of the degrader compound of Formula IA-5, s=2. In other embodiments of the degrader compound of Formula IA-5, s=3.

In some embodiments of the degrader compound of Formula IA-5, R^{k}=H. In some embodiments of the degrader compound of Formula IA-5, R^{k}=deuterium. In some embodiments of the degrader compound of Formula IA-5, R^{k}=F. In some embodiments of the degrader compound of Formula IA-5, R^{k}═C₁₋₃ alkyl, for example, C₁ alkyl, C₂ alkyl, C₃ alkyl, —CH₃, —CH₂CH₃, and the like. In some embodiments of the degrader compound of Formula IA-5, R^{k}=C₁₋₃ haloalkyl, for example, C₁ haloalkyl, C₂ haloalkyl, C₃ haloalkyl, —CF₃, —CH₂CF₃, and the like. In some embodiments of the degrader compound of Formula IA-5, R^{k}=H. or C₁₋₄ alkoxyl, for example, C₁ alkoxyl, C₂ alkoxyl, C₃ alkoxyl, —OCH₃, —OCH₂CH₃, and the like.

In some aspects, the degrader compound of Formula IA is a degrader compound of Formula IA-6, Formula IA-6a or Formula IA-6b:

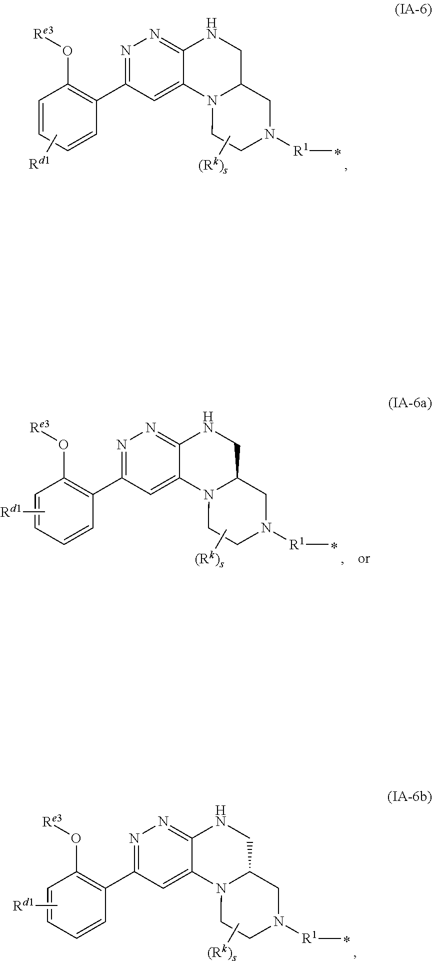

wherein R^{k}=H, deuterium, F, C₁₋₃ alkyl, C₁₋₃ haloalkyl, or C₁₋₄ alkoxyl; and s=0-3, and wherein R^{c1}, R^{d1}, R^{e3}, and R¹ are as described above for Formula IA.

In some embodiments, the degrader compound is a degrader compound of Formula IA-6. In some embodiments, the degrader compound is a degrader compound of Formula IA-6a. In some embodiments, the degrader compound is a degrader compound of Formula IA-6b.

In some embodiments of the degrader compound of Formula IA-6, IA-6a or IA-6b, s=0. In some embodiments of the degrader compound of Formula IA-6, IA-6a or IA-6b, s=1. In other embodiments of the degrader compound of Formula IA-6, IA-6a or IA-6b, s=2. In other embodiments of the degrader compound of Formula IA-6, IA-6a or IA-6b, s=3.

In some embodiments of the degrader compound of Formula IA-6, IA-6a or IA-6b, R^{k}=H. In some embodiments of the degrader compound of Formula IA-6, IA-6a or IA-6b, R^{k}=deuterium. In some embodiments of the degrader compound of Formula IA-6, IA-6a or IA-6b, R^{k}=F. In some embodiments of the degrader compound of Formula IA-6, IA-6a or IA-6b, R^{k}=C₁₋₃ alkyl, for example, C₁ alkyl, C₂ alkyl, C₃ alkyl, —CH₃, —CH₂CH₃, and the like. In some embodiments of the degrader compound of Formula IA-6, IA-6a or IA-6b, R^{k}≡C₁₋₃ haloalkyl, for example, C₁ haloalkyl, C₂ haloalkyl, C₃ haloalkyl, —CF₃, —CH₂CF₃, and the like. In some embodiments of the degrader compound of Formula IA-6, IA-6a or IA-6b, R^{k}=H. or C₁₋₄ alkoxyl, for example, C₁ alkoxyl, C₂ alkoxyl, C₃ alkoxyl, —OCH₃, —OCH₂CH₃, and the like.

### ULM

In some aspects, the ULM moiety in the degrader compounds of the disclosure is a small molecule E3 Ubiquitin Ligase binding moiety that binds a Von Hippel-Lindau E3 Ubiquitin Ligase (VHL) or a Cereblon E3 Ubiquitin Ligase (CRBN). Such ULM moieties that bind to VHL are known to those of skill in the art. Methods of determining whether a small molecule binds a Von Hippel-Lindau E3 Ubiquitin Ligase are known in the art. Such ULM moieties that bind to CRBN are known to those of skill in the art. Methods of determining whether a small molecule binds a Cereblon E3 Ubiquitin Ligase are known in the art, for example, see Lai A. C., Crews C. M. Nat Rev Drug Discov. 2017; 16(2):101-114.

### Von Hippel-Lindau E3 Ubiquitin Ligase (VHL)

In some aspects, the ULM moiety in the degrader compounds of the disclosure is a small molecule E3 Ubiquitin Ligase binding moiety that binds a Von Hippel-Lindau E3 Ubiquitin Ligase (VHL). Such ULM moieties that bind to VHL are known to those of skill in the art. Methods of determining whether a small molecule binds a Von Hippel-Lindau E3 Ubiquitin Ligase are known in the art.

In some embodiments, the ULM is a previously described ULM. In some embodiments, the ULM is a ULM moiety described in U.S. Patent Application Publication No. 2019/0300521, the entirety of which is incorporated by reference herein. In other embodiments, the ULM is a ULM moiety described in U.S. Patent Application Publication No. 2019/0255066, the entirety of which is incorporated by reference herein. In other embodiments, the ULM is a ULM moiety described in WO 2019/084030, the entirety of which is incorporated by reference herein. In other embodiments, the ULM is a ULM moiety described in WO 2019/084026, the entirety of which is incorporated by reference herein.

In some embodiments, the ULM is a moiety having the Formula ULM-I-VHL

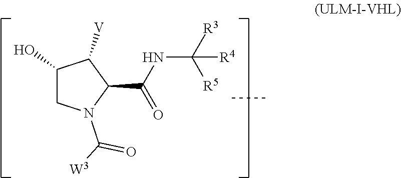

wherein
[symbol] indicates the position of attachment of the ULM to R¹;
V is H or F;
R³ is optionally substituted phenyl, optionally substituted napthyl, or an optionally substituted 5-10 membered heteroaryl;
one of R⁴ or R⁵ is H, deuterium, haloalkyl, optionally substituted alkyl, optionally substituted cycloalkyl, optionally substituted heterocycloalkyl, —COR^{dv}, or CONR^{e1}R^{e2}; the other of R⁴ or R⁵ is H or deuterium; or R⁴ and R⁵, together with the carbon atom to which they are both attached, form an optionally substituted 3-5 membered cycloalkyl, or heterocyclyl;
W³ is an optionally substituted aryl, optionally substituted heteroaryl, or

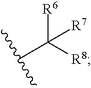

R⁶ and R⁷ are independently H, deuterium, optionally substituted alkyl, optionally substituted cycloalkyl, or optionally substituted haloalkyl, or R⁶, R⁷, and the carbon atom to which they are attached form an optionally substituted cycloalkyl or optionally substituted heterocyclyl;
R⁸ is an optionally substituted heterocyclyl, optionally substituted heteroaryl, optionally substituted aryl, —C(O)NR^{av}R^{bv}, —NR^{av}R^{bv},

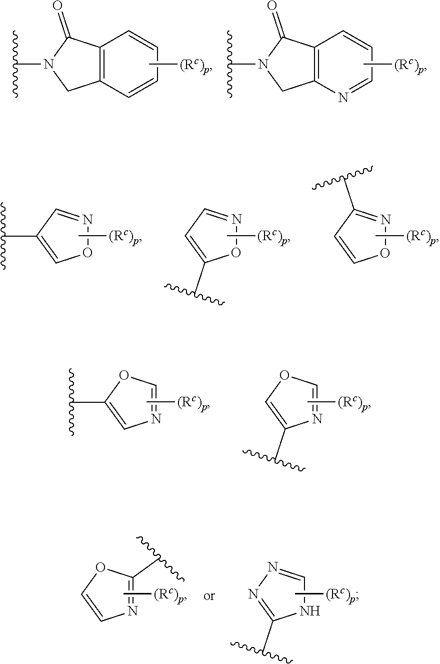

R^{av} is H or optionally substituted alkyl;
R^{bv} is H, —C(O)—* wherein * is a point of attachment to R¹, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl)carbonyl, or optionally substituted aralkyl;
each R^{c} is independently H, halo, optionally substituted alkoxy, cyano, optionally substituted alkyl, haloalkyl, or haloalkoxy;
each R^{dv} is independently H, optionally substituted alkyl or NR^{e1}R^{e2};
each R^{e1} and R^{e2} is independently H, deuterium, or optionally substituted alkyl, or R^{e1} and R^{e2} together with the nitrogen atom to which they are attached form a 4-7 membered heterocyclyl; and
p is 0, 1, 2, 3, or 4.

In some embodiments of ULM-I-VHL, V is H. In other embodiments of ULM-I-VHL, V is F.

In some embodiments of ULM-I-VHL, R³ is optionally substituted phenyl having the formula:

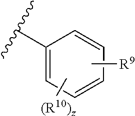

wherein
R⁹ is H, deuterium, halo, —CN, —OH, —NO₂, —NR^{e1}R^{e2}, —OR^{e1}, —CONR^{e1}R^{e2}, —NR^{e1}COR^{e2}, —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R¹⁰ is H, deuterium, halo, CN, optionally substituted alkyl, optionally substituted haloalkyl, hydroxy, optionally substituted alkoxy, or optionally substituted haloalkoxy; and
z is 0, 1, 2, 3, or 4.

In some embodiments wherein R³ is optionally substituted phenyl, R¹⁰ is —F or —OCH₃. In some embodiments wherein R³ is optionally substituted phenyl, R⁹ is —CN. In some embodiments wherein R³ is optionally substituted phenyl, R⁹ is an optionally substituted heteroaryl. In some embodiments wherein R³ is optionally substituted phenyl, R⁹ is

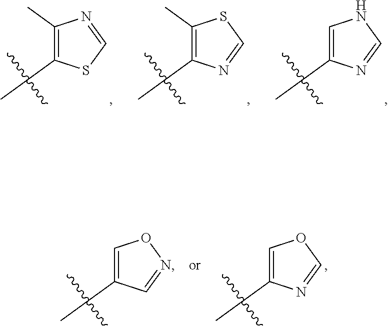

each optionally substituted.

In other embodiments wherein R³ is optionally substituted phenyl, R⁹ is

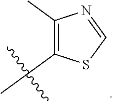 [SMILES: *c1scnc1C]

In other embodiments wherein R³ is optionally substituted phenyl, R⁹ is

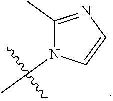 [SMILES: *n1ccnc1C]

In other embodiments, R³ is

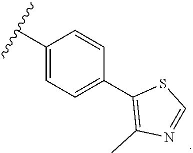 [SMILES: *c1ccc(-c2scnc2C)cc1]

In some embodiments wherein R³ is optionally substituted phenyl, R¹⁰ is hydroxy, halogen, —NH(C₁-C₄alkyl), or C₁-C₆alkoxy, and z is 0, 1, 2, 3, or 4.

In some embodiments of ULM-I-VHL, one of R⁴ or R⁵ is H, and the other of R⁴ or R⁵ is H or optionally substituted alkyl. In other embodiments of ULM-I-VHL, one of R⁴ or R⁵ is H, and the other of R⁴ or R⁵ is optionally substituted C₁-C₆alkyl. In other embodiments of ULM-I-VHL, one of R⁴ or R⁵ is H, and the other of R⁴ or R⁵ is C₁-C₆alkyl. In other embodiments of ULM-I-VHL, one of R⁴ or R⁵ is H, and the other of R⁴ or R⁵ is —CH₃. In other embodiments of ULM-I-VHL, one of R⁴ or R⁵ is H, and the other of R⁴ or R⁵ is —CH₂OH. In other embodiments of ULM-I-VHL, both R⁴ and R⁵ are H.

In some embodiments of ULM-I-VHL, W³ is

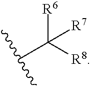

In some embodiments of ULM-I-VHL, R⁶ is H. In some embodiments of ULM-I-VHL, R⁷ is H, or optionally substituted alkyl. In some embodiments of ULM-I-VHL, R⁷ is H. In some embodiments of ULM-I-VHL, R⁷ is optionally substituted alkyl. In some embodiments of ULM-I-VHL, R⁷ is optionally substituted C₁-C₆alkyl. In some embodiments of ULM-I-VHL, R⁷ is C₁-C₆alkyl. In some embodiments of ULM-I-VHL, R⁷ is C₁-C₆alk-OH, C₁-C₆alk-NH₂, —C₁-C₆alk-CONH—*, or —C₁-C₆alk-NHCO—* wherein * is a point of attachment to R¹. In some embodiments of ULM-I-VHL, R⁷ is -t-butyl or -isopropyl. In some embodiments of ULM-I-VHL, R⁷ is -t-butyl. In some embodiments of ULM-I-VHL, R⁷ is -isopropyl.

In some embodiments of ULM-I-VHL, R⁸ is NR^{av}R^{bv}. In some embodiments, R^{av} is H or optionally substituted alkyl. In some embodiments, R^{av} is H. In some embodiments, R^{bv} is H, optionally substituted alkyl, —C(O)—* wherein * is a point of attachment to R¹, optionally substituted (cycloalkyl)carbonyl, or optionally substituted alkylcarbonyl. In some embodiments, R^{bv} is optionally substituted alkylcarbonyl. In some embodiments, R^{bv} is —C(O)—* wherein * is a point of attachment to R¹. In some embodiments of ULM-I-VHL, R⁸ is CONR^{av}R^{bv}.

In some embodiments of ULM-I-VHL, R⁸ is

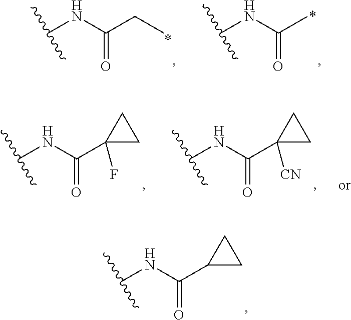

wherein * is a point of attachment to R¹. In some embodiments of ULM-I-VHL, R⁸ is

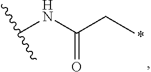 [SMILES: *NC(=O)CC]

wherein * is a point of attachment to R¹. In some embodiments of ULM-I-VHL, R⁸ is

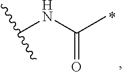 [SMILES: *NC(C)=O]

wherein * is a point of attachment to R¹. In some embodiments of ULM-I-VHL, R⁸is

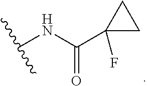 [SMILES: *NC(=O)C1(F)CC1]

In some embodiments, R⁸ is —NH—* wherein * is a point of attachment to R¹. In some embodiments of ULM-I-VHL, R⁸ is optionally substituted heteroaryl.

In some embodiments of ULM-I-VHL, R⁸ is

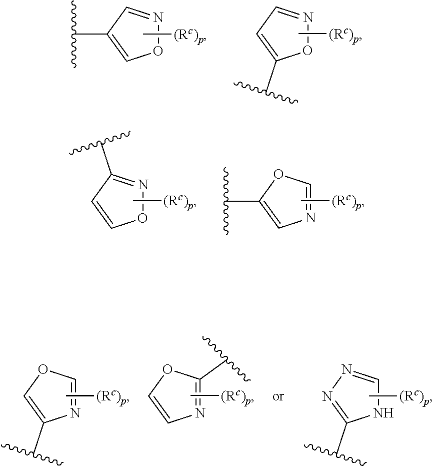

wherein each R^{c} is independently halo, optionally substituted alkoxy, cyano, optionally substituted alkyl, haloalkyl, or haloalkoxy, and p is 0, 1, or 2.

In some embodiments, R⁸ is

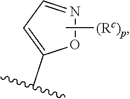

wherein each R^{c} is independently halo, optionally substituted alkoxy, cyano, optionally substituted alkyl, haloalkyl, or haloalkoxy, and p is 0, 1, or 2.

In some embodiments, R⁸ is

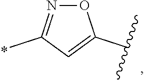 [SMILES: *c1cc(C)no1]

wherein * is a point of attachment to R¹. In some embodiments, R⁸ is

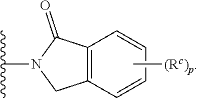

In some embodiments, R⁸ is

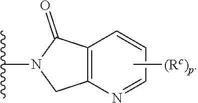

In some embodiments, R⁸ is

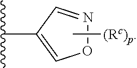

In some embodiments, R⁸ is

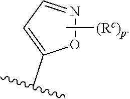

In some embodiments, R⁸ is

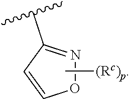

In some embodiments, R⁸ is

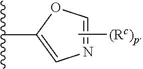

In some embodiments, R⁸ is

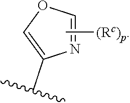

In some embodiments, R⁸ is

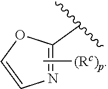

In some embodiments, R⁸ is

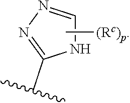

In some embodiments, ULM-I-VHL is a compound of formula:

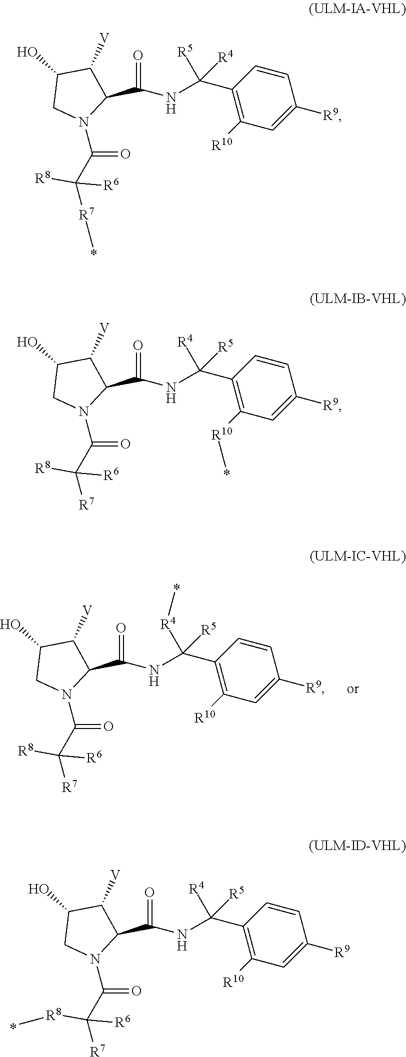

* is a point of attachment of the ULM to R¹.

In some embodiments of ULM-IA-VHL, ULM-IB-VHL, ULM-IC-VHL, or ULM-ID-VHL, R⁹ is optionally substituted

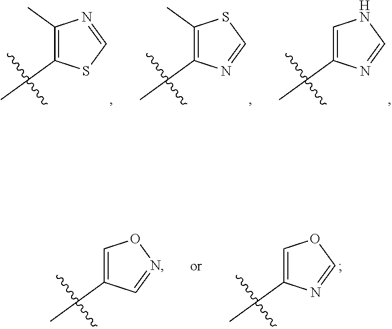

and R¹⁰ is H, deuterium, hydroxy, halogen, aminoC₁₋₄alkyl, or C₁₋₄alkyloxy.

In some embodiments, the ULM is a moiety having the Formula ULM-I-VHL-1:

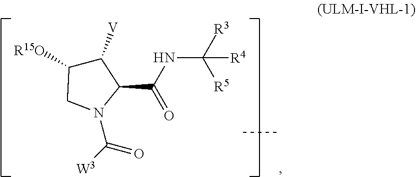

wherein
the dashed line ( [symbol] ) indicates the position of attachment of ULM-I-VHL-1 to R¹;
R¹⁵ is hydrogen or —PO₃H₂; and all of the other variables have the same scope as set forth above with respect to ULM-I-VHL.

In some embodiments, R¹⁵ in ULM-I-VHL-1 is hydrogen. In other embodiments, R¹⁵ in ULM-I-VHL-1 is —PO₃H₂.

In some embodiments, the ULM is a moiety having the Formula ULM-II-VHL

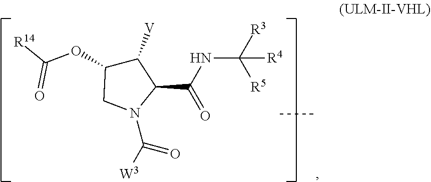

wherein
[symbol] indicates the position of attachment of the ULM to R¹;
R¹⁴ is C₁-C₆alkyl, such as, for example, —CH₃, —CH₂CH₃, —CH(CH₃)₂, and the like; and all of the other variables have the same scope as set forth above with respect to ULM-I-VHL.

In some embodiments, R¹⁴ in ULM-II-VHL is —CH₃. In other embodiments, R¹⁴ in ULM-II-VHL is —CH(CH₃)₂.

In some aspects, the degrader compounds of Formula I are those having the formula IA-7-VHL or IA-8-VHL:

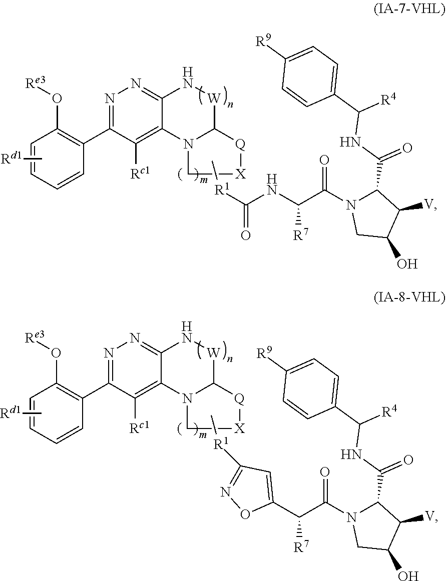

wherein V is H or F;
W is optionally substituted —CH₂—, —C(O)—, —S(O)—, or —S(O)₂—; wherein when n=2 or 3,
only one W may be —C(O)—, —S(O)—, or —S(O)₂—;
n=0-3;
m=1-3;
R^{k}=H, deuterium, F, C₁₋₃ alkyl, C₁₋₃ haloalkyl, or C₁₋₄ alkoxyl;
R^{c1} and R^{d1} are independently H, deuterium, Halo, C₁₋₃ alkyl, C₁₋₃ haloalkyl, or C₁₋₄ alkoxyl;
R^{e3} is H, —C(O)R^{f}, —CH₂—O—P(O)(OR^{g})₂, or —P(O)(OR^{g})₂; wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or
R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}, —(CR^{1a}R^{1b})₁₋₅-A-(CR^{1a}R^{1b})₁₋₅— wherein A is O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})₁₋₅—(CR^{1a}═CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b}groups)-, -(3-11 membered cycloalkyl optionally substituted with 0-6 R^{1a} and/or R^{1b}groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-(3-11 membered heterocyclyl optionally substituted with 0-6 R^{1a} and/or R^{1b} groups)-A- wherein A is O, S, or NR^{1c}, —(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b}groups)-(CR^{1a}R^{1b})₁₋₅-A- wherein each A is independently O, S, or NR^{1c}, —(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}, —(CR^{1a}R^{1b})₁₋₅-A-(CR^{1a}R^{1b})₁₋₅-A- wherein A is O, S, or NR^{1c}—(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}—(CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}, —(CR^{1a}R^{1b})₁₋₅—(C≡C—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b}groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}, -(3-11 membered heterocyclyl optionally substituted with 0-6 R^{1a} and/or R^{1b} groups)-(CR^{1a}R^{1b})₁₋₅-A- wherein A is O, S, or NR^{1c}; —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}, -(3-11 membered cycloalkyl optionally substituted with 0-6 R^{1a} and/or R^{1b} groups)-(CR^{1a}R^{1b})₁₋₅—, or -(3-11 membered heterocyclyl optionally substituted with 0-6 R^{1a} and/or R^{1b} groups)-(CR^{1a}R^{1b})₁₋₅—;
R⁴ is H, optionally substituted alkyl, optionally substituted C₁-C₆alkyl, or —CH₃;
R⁷ is optionally substituted alkyl, preferably optionally substituted C₁-C₆alkyl, and more preferably C₁-C₆alkyl; and
R⁹ is H, deuterium, halo, —CN, —OH, —NO₂, —NR^{e1}R^{e2}, —OR^{e1}, —CONR^{e1}R^{e2}, —NR^{e1}COR^{e2}, —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R^{1a}, R^{1b}, R^{1c}, and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC1-C₈alkyl)(C₁-C₈alkyl), —P(O)(OC1-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups; and
each R^{e1} and R^{e2} is independently H, deuterium, or optionally substituted alkyl, or R^{e1}
and R^{e2} together with the nitrogen atom to which they are attached form a 4-7 membered heterocyclyl.

In some aspects, the degrader compounds of Formula I are those having the formula IA-9-VHL or IA-10-VHL:

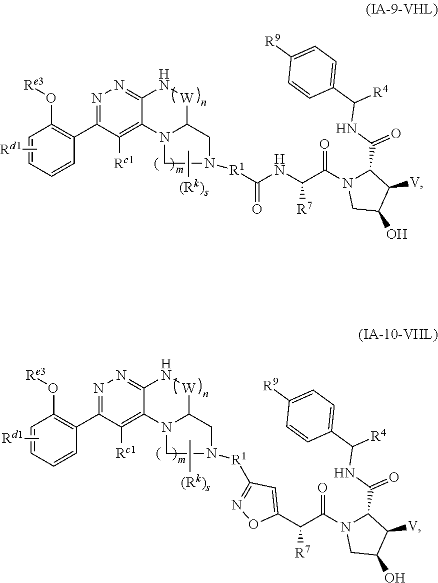

wherein V is H or F;
W is optionally substituted —CH₂—, —C(O)—, —S(O)—, or —S(O)₂—; wherein when n=2 or 3, only one W may be —C(O)—, —S(O)—, or —S(O)₂—;
n=0-3;
m=1-3;
R^{k}=H, deuterium, F, C₁₋₃ alkyl, C₁₋₃ haloalkyl, or C₁₋₄ alkoxyl;
s=0-3;
R^{c1} and R^{d1} are independently H, deuterium, Halo, C₁₋₃ alkyl, C₁₋₃ haloalkyl, or C₁₋₄ alkoxyl;
R^{e3} is H, —C(O)R, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b} groups, —(CR^{1a}R^{1b})₁₋₅, —(CR^{1a}═CR^{1b})—(CR^{1a}R^{1b})₁₋₅-A- wherein A is O, S, or NR^{1c}, —(CR^{1a}R^{1b})₁₋₅-A-(CR^{1a}R^{1b})₁₋₅— wherein A is O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})₁₋₅—(CR^{1a}═CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b}groups)-, -(3-11 membered cycloalkyl optionally substituted with 0-6 R^{1a} and/or R^{1b}groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-(3-11 membered heterocyclyl optionally substituted with 0-6 R^{1a} and/or R^{1b} groups)-A- wherein A is O, S, or NR^{1c}, —(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b}groups)-(CR^{1a}R^{1b})₁₋₅-A- wherein each A is independently O, S, or NR^{1c}, —(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(CR^{1a}R^{1b})₁₋₅-A- wherein A is O, S, or NR^{1c}—(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}—(CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}—(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b}groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}, -(3-11 membered cycloalkyl optionally substituted with 0-6 R^{1a} and/or R^{1b} groups)-(CR^{1a}R^{1b})₁₋₅—, or -(3-11 membered heterocyclyl optionally substituted with 0-6 R^{1a} and/or R^{1b} groups)-(CR^{1a}R^{1b})₁₋₅; or R^{j} is -(3-11 membered heterocyclyl optionally substituted with 0-6 R^{1a} and/or R^{1b} groups)-(CO)—(CR^{1a}R^{1b})₁₋₅-A- wherein A is O, S, or NR^{1c}; -(3-11 membered heterocyclyl optionally substituted with 0-6 R^{1a} and/or R^{1b} groups)-(CO)-A-(CR^{1a}R^{1b})₁₋₅— wherein A is O, S, or NR^{1c}; -A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}; -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-A-(CR^{1a}R^{1b})₁₋₅-A- wherein each A is independently O, S, or NR^{1c}; -(heteroaryl optionally substituted with 0-4 R^{1a} and/or R^{1b} groups)-A-(CR^{1a}R^{1b})₁₋₅— wherein A is O, S, or NR^{1c}; -(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)—(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or —(CO)-(3-11 membered cycloalkyl optionally substituted with 0-6 R^{1a} and/or R^{1b} groups)-(CR^{1a}R^{1b})₁₋₅-A- wherein A is O, S, or NR^{1c};
R⁴ is H, optionally substituted alkyl, optionally substituted C₁-C₆alkyl, or —CH₃;
R⁷ is optionally substituted alkyl, preferably optionally substituted C₁-C₆alkyl, and more preferably C₁-C₆alkyl; and
R⁹ is H, deuterium, halo, —CN, —OH, —NO₂, —NR^{e1}R^{e2}, —OR^{e1}, —CONR^{e1}R^{e2}, —NR^{e1}COR^{e2}, —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R^{1a}, R^{1b}, R^{1c}, and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC1-C₈alkyl)(C₁-C₈alkyl), —P(O)(OC1-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups; and
each R^{e1} and R^{e2} is independently H, deuterium, or optionally substituted alkyl, or R^{e1} and R^{e2} together with the nitrogen atom to which they are attached form a 4-7 membered heterocyclyl.

In some embodiments of the degrader compound of Formula IA-9-VHL or IA-10-VHL, n=1. In other embodiments of the degrader compound of Formula IA-9-VHL or IA-10-VHL, n=2. In other embodiments of the degrader compound of Formula IA-9-VHL or IA-10-VHL, n=3.

In some embodiments of the degrader compound of Formula IA-7-VHL or IA-8-VHL, m=1. In other embodiments of the degrader compound of Formula IA-7-VHL or IA-8-VHL, m=2. In other embodiments of the degrader compound of Formula IA-7-VHL or IA-8-VHL, m=3.

In some embodiments of the degrader compound of Formula IA-9-VHL or IA-10-VHL, s=0. In some embodiments of the degrader compound of Formula IA-9-VHL or IA-10-VHL, s=1. In other embodiments of the degrader compound of Formula IA-9-VHL or IA-10-VHL, s=2.

In other embodiments of the degrader compound of Formula IA-9-VHL or IA-10-VHL, p=3.

In some embodiments of the degrader compound of Formula IA-9-VHL or IA-10-VHL, R^{k}=H. In some embodiments of the degrader compound of Formula IA-9-VHL or IA-10-VHL, R^{k}=deuterium. In some embodiments of the degrader compound of Formula IA-9-VHL or IA-10-VHL, R^{k}=F. In some embodiments of the degrader compound of Formula IA-9-VHL or IA-10-VHL, R^{k}═C₁₋₃ alkyl, for example, C₁ alkyl, C₂ alkyl, C₃ alkyl, —CH₃, —CH₂CH₃, and the like.

In some embodiments of the degrader compound of Formula IA-9-VHL or IA-10-VHL, R^{k}═C₁₋₃ haloalkyl, for example, C₁ haloalkyl, C₂ haloalkyl, C₃ haloalkyl, —CF₃, —CH₂CF₃, and the like. In some embodiments of the degrader compound of Formula IA-9-VHL or IA-10-VHL, R^{k}=H. or C₁₋₄ alkoxyl, for example, C₁ alkoxyl, C₂ alkoxyl, C₃ alkoxyl, —OCH₃, —OCH₂CH₃, and the like.

In some embodiments of the degrader compound of Formula IA-9-VHL or IA-10-VHL, R^{c1} and R^{d1} are each H. In some embodiments of the degrader compound of Formula IA-9-VHL or IA-10-VHL, R^{e3} is H. In some embodiments of the degrader compound of Formula IA-9-VHL or IA-10-VHL, R^{c1}, R^{d1}, and R^{e3} are each H.

In some aspects, the degrader compounds of Formula I are degrader compounds of Formula IA-9a-VHL or IA-10a-VHL.

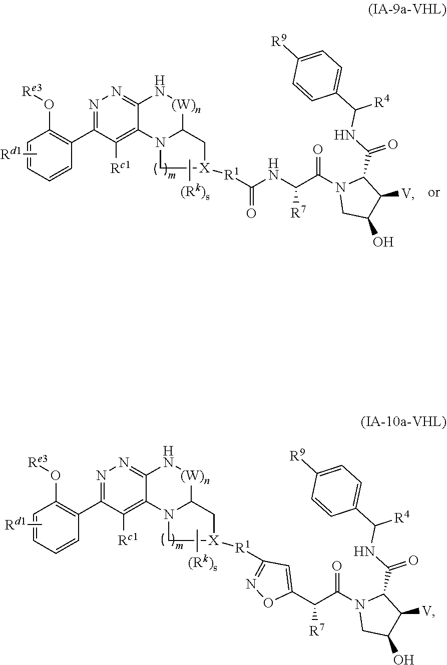

wherein X is N or CH, and the other variables are as set forth above with respect to Formula IA-9-VHL and IA-10-VHL.

In some aspects, the degrader compounds of Formula I are those having the formula IA-11-VHL or IA-12-VHL:

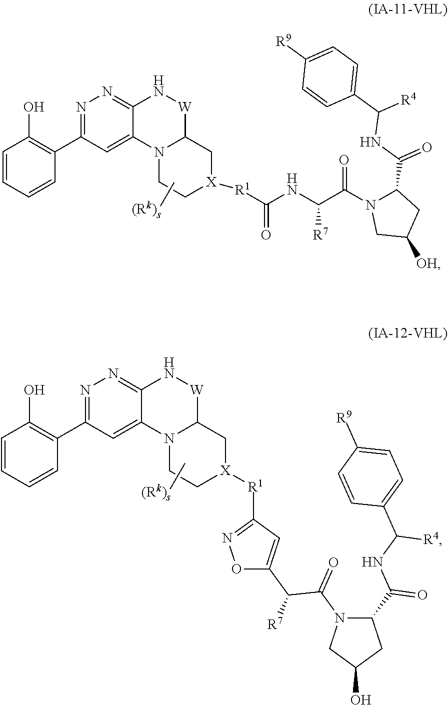

wherein
W is —CH₂— or —CH(CH₃)—
X is N or CH;
R¹ is a covalent bond;
   3-11 membered cycloalkyl optionally substituted with 0-6 R^{1a} and/or R^{1b} groups; 3-11 membered heterocyclyl optionally substituted with 0-6 R^{1a} and/or R^{1b} groups;
   —(CR^{1a}R^{1b})₁₋₅;
   —(CR^{1a}═CR^{1b})—;
   —(CR^{1a}R^{1b})₁₋₅-A- wherein A is O, S, or NR^{1c};
   —(CR^{1a}R^{1b})₁₋₅-A-(CR^{1a}R^{1b})₁₋₅— wherein A is O, S, or NR^{1c};
   —(CR^{1a}R^{1b})₁₋₅-A-(CR^{1a}R^{1b})₁₋₅-A- wherein A is O, S, or NR^{1c};
   —(CR^{1a}R^{1b})₁₋₅—(CR^{1a}═CR^{1b})—(CR^{1a}R^{1b})₁₋₅—;
   —(CR^{1a}R^{1b})₁₋₅—(CR^{1a}═CR^{1b})—(CR^{1a}R^{1b})₁₋₅-A- wherein A is O, S, or NR^{1c};
   —(CR^{1a}R^{1b})₁₋₅—(C≡C)—(CR^{1a}R^{1b})₁₋₅—;
   —(CR^{1a}R^{1b})₁₋₅—(C≡C)—(CR^{1a}R^{1b})₁₋₅-A- wherein A is O, S, or NR^{1c};
   —(C≡C)—(CR^{1a}R^{1b})₁₋₅-A-(CR^{1a}R^{1b})₁₋₅- wherein A is O, S, or NR^{1c};
   —(C≡C)—(CR^{1a}R^{1b})₁₋₅;
   —(CR^{1a}R^{1b})₁₋₅-(3-11 membered cycloalkyl optionally substituted with 0-6 R^{1a} and/or R^{1b}groups)-;
   —(CR^{1a}R^{1b})₁₋₅-(3-11 membered heterocyclyl optionally substituted with 0-6 R^{1a} and/or R^{1b} groups)-;
   -(3-11 membered cycloalkyl optionally substituted with 0-6 R^{1a} and/or R^{1b} groups)-(CR^{1a}R^{1b})₁₋₅—;
   -(3-11 membered cycloalkyl optionally substituted with 0-6 R^{1a} and/or R^{1b} groups)-(CR^{1a}R^{1b})₁₋₅-A- wherein A is O, S, or NR^{1c};
   -(3-11 membered heterocyclyl optionally substituted with 0-6 R^{1a} and/or R^{1b} groups)-(CR^{1a}R^{1b})₁₋₅-A- wherein A is O, S, or NR^{1c};
   -(3-11 membered heterocyclyl optionally substituted with 0-6 R^{1a} and/or R^{1b} groups)-(CR^{1a}R^{1b})₁₋₅—;
   —(CR^{1a}R^{1b})₁₋₅-(3-11 membered cycloalkyl optionally substituted with 0-6 R^{1a} and/or R^{1b}groups)-A- wherein A is O, S, or NR^{1c};
   —(CR^{1a}R^{1b})₁₋₅-(3-11 membered heterocyclyl optionally substituted with 0-6 R^{1a} and/or R^{1b}groups)-A- wherein A is O, S, or NR^{1c};
   —(CR^{1a}R^{1b})₁₋₅-(3-11 membered cycloalkyl optionally substituted with 0-6 R^{1a} and/or R^{1b}groups)-(CR^{1a}R^{1b})₁₋₅;
   —(CR^{1a}R^{1b})₁₋₅-(3-11 membered cycloalkyl optionally substituted with 0-6 R^{1a} and/or R^{1b}groups)-A- wherein A is O, S, or NR^{1c};
   —(CR^{1a}R^{1b})₁₋₅-(3-11 membered cycloalkyl optionally substituted with 0-6 R^{1a} and/or R^{1b}groups)-(CR^{1a}R^{1b})₁₋₅-A- wherein A is O, S, or NR^{1c};
   —(CR^{1a}R^{1b})₁₋₅-A-(3-11 membered cycloalkyl optionally substituted with 0-6 R^{1a} and/or R^{1b} groups)- wherein A is O, S, or NR^{1c};
   —(CR^{1a}R^{1b})₁₋₅-(3-11 membered heterocyclyl optionally substituted with 0-6 R^{1a} and/or R^{1b} groups)-(CR^{1a}R^{1b})₁₋₅;
   —(CR^{1a}R^{1b})₁₋₅-(3-11 membered heterocyclyl optionally substituted with 0-6 R^{1a} and/or R^{1b}groups)-(CR^{1a}R^{1b})₁₋₅-A- wherein A is O, S, or NR^{1c};
   —(CR^{1a}R^{1b})₁₋₅-(3-11 membered heterocyclyl optionally substituted with 0-6 R^{1a} and/or R^{1b}groups)-A- wherein A is O, S, or NR^{1c};
   —(CR^{1a}R^{1b})₁₋₅-A-(3-11 membered heterocyclyl optionally substituted with 0-6 R^{1a} and/or R^{1b} groups)- wherein A is O, S, or NR^{1c};
   —(CR^{1a}R^{1b})₁₋₅-(3-11 membered cycloalkyl optionally substituted with 0-6 R^{1a} and/or R^{1b}groups)-(CR^{1a}R^{1b})₁₋₅-A- wherein A is O, S, or NR^{1c};
   —(CR^{1a}R^{1b})₁₋₅-A-(3-11 membered cycloalkyl optionally substituted with 0-6 R^{1a} and/or R^{1b} groups)- wherein A is O, S, or NR^{1c};
   —(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c};
   —(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c};
   —(CR^{1a}R^{1b})₁₋₅-A-(CR^{1a}R^{1b})₁₋₅-A- wherein A is O, S, or NR^{1c};
   —(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein each A is independently O, S, or NR^{1c};
   —(CR^{1a}R^{1b})₁₋₅-A-(CO) wherein A is O, S, or NR^{1c};
   —(CR^{1a}R^{1b})₁₋₅—(CR^{1a}═CR^{1b})—(CR^{1a}R^{1b})₁₋₅-A-(CO)— wherein A is O, S, or NR^{1c};
   —(CR^{1a}R^{1b})₁₋₅—(C≡C)—(CR^{1a}R^{1b})₁₋₅-A-(CO)— wherein A is O, S, or NR^{1c};
   —(CR^{1a}R^{1b})₁₋₅-(3-11 membered cycloalkyl optionally substituted with 0-6 R^{1a} and/or R^{1b}groups)-(CR^{1a}R^{1b})₁₋₅-A-(CO)— wherein A is O, S, or NR^{1c};
   —(CR^{1a}R^{1b})₁₋₅-A-(CO)-(3-11 membered cycloalkyl optionally substituted with 0-6 R^{1a} and/or R^{1b} groups)- wherein A is O, S, or NR^{1c};
   —(CR^{1a}R^{1b})₁₋₅-(3-11 membered heterocyclyl optionally substituted with 0-6 R^{1a} and/or R^{1b}groups)-(CR^{1a}R^{1b})₁₋₅-A-(CO)— wherein A is O, S, or NR^{1c};
   —(CR^{1a}R^{1b})₁₋₅-A-(CO)-(3-11 membered heterocyclyl optionally substituted with 0-6 R^{1a} and/or R^{1b} groups)- wherein A is O, S, or NR^{1c};
   —(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c};
   -(3-11 membered cycloalkyl optionally substituted with 0-6 R^{1a} and/or R^{1b} groups)-CO—(CR^{1a}R^{1b})₁₋₅-A- wherein A is O, S, or NR^{1c};
   —(CR^{1a}R^{1b})₁₋₅-(3-11 membered cycloalkyl optionally substituted with 0-6 R^{1a} and/or R^{1b}groups)-(CR^{1a}R^{1b})₁₋₅-A-(CO)— wherein A is O, S, or NR^{1c};
   —(CR^{1a}R^{1b})₁₋₅-(3-11 membered heterocyclyl optionally substituted with 0-6 R^{1a} and/or R^{1b}groups)-(CR^{1a}R^{1b})₁₋₅-A-(CO)— wherein A is O, S, or NR^{1c};
   -(3-11 membered cycloalkyl optionally substituted with 0-6 R^{1a} and/or R^{1b} groups)-(CR^{1a}R^{1b})₁₋₅—;
   -(3-11 membered heterocyclyl optionally substituted with 0-6 R^{1a} and/or R^{1b} groups)-(CR^{1a}R^{1b})₁₋₅—; -A-(3-11 membered heterocyclyl optionally substituted with 0-6 R^{1a} and/or R^{1b} groups)-(CR^{1a}R^{1b})₁₋₅-A- wherein each A is independently O, S, or NR^{1c};
   -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b}groups)-(CR^{1a}R^{1b})₁₋₅-A- wherein A is O, S, or NR^{1c};
   -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c};
   -(3-11 membered heterocyclyl optionally substituted with 0-6 R^{1a} and/or R^{1b} groups)-A-(CR^{1a}R^{1b})₁₋₅-A- wherein each A is independently O, S, or NR^{1c};
   -(3-11 membered heterocyclyl optionally substituted with 0-6 R^{1a} and/or R^{1b} groups)-(CO)-A-(CR^{1a}R^{1b})₁₋₅— wherein A is O, S, or NR^{1c};
-(heteroaryl optionally substituted with 0-4 R^{1a} and/or R^{1b} groups)-A-(CR^{1a}R^{1b})₁₋₅— wherein A is O, S, or NR^{1c};
   -(3-11 membered cycloalkyl optionally substituted with 0-6 R^{1a} and/or R^{1b} groups)-(CO)—(CR^{1a}R^{1b})₁₋₅-A- wherein A is O, S, or NR^{1c};
   -(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or
   —(CO)-(3-11 membered cycloalkyl optionally substituted with 0-6 R^{1a} and/or R^{1b} groups)-(CR^{1a}R^{1b})₁₋₅-A- wherein A is O, S, or NR^{1c};
   R⁴ is H, —CH₃, or —CH₂OH;
   R⁷ is C₁-C₆alkyl, preferably —C(CH₃)₃ or —CH(CH₃)₂; and
   R⁹ is —CN or optionally substituted heteroaryl, preferably,

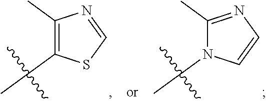

R^{1a}, R^{1b}, R^{1c}, and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups.

In some embodiments, W is —CH₂—. In some embodiments, X is —N. In other embodiments, X is —CH. In some embodiments, R⁴ is —CH₃. In some embodiments, R⁷ is ably —C(CH₃)₃ or —CH(CH₃)₂. In some embodiments, R^{k} is —CH₃ and s=1. In some embodiments, s=0. In some embodiments, R⁹ is S

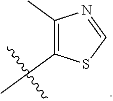 [SMILES: *c1scnc1C]

In some embodiments of the degrader compound of formula I, such as formula IA-1I-VHL or formula IA-12-VHL, R¹ is:
—(CR^{1a}R^{1b})₁₋₅;
—(CR^{1a}R^{1b})₁₋₅-A- wherein A is O, S, or NR^{1c};
—(CR^{1a}R^{1b})₁₋₅-A-(CR^{1a}R^{1b})₁₋₅— wherein A is O, S, or NR^{1c};
-(3-11 membered heterocyclyl optionally substituted with 0-6 R^{1a} and/or R^{1b} groups)-(CO)—(CR^{1a}R^{1b})₁₋₅-A- wherein A is O, S, or NR^{1c};
-(3-11 membered heterocyclyl optionally substituted with 0-6 R^{1a} and/or R^{1b} groups)-(CO)-A-(CR^{1a}R^{1b})₁₋₅— wherein A is O, S, or NR^{1c};
—(CR^{1a}R^{1b})₁₋₅-(3-11 membered cycloalkyl optionally substituted with 0-6 R^{1a} and/or R^{1b}groups)-A- wherein A is O, S, or NR^{1c};
—(CR^{1a}R^{1b})₁₋₅-(3-11 membered heterocyclyl optionally substituted with 0-6 R^{1a} and/or R^{1b}groups)-(CR^{1a}R^{1b})₁₋₅;
—(CR^{1a}R^{1b})₁₋₅-(3-11 membered heterocyclyl optionally substituted with 0-6 R^{1a} and/or R^{1b}groups)-(CR^{1a}R^{1b})₁₋₅-A- wherein A is O, S, or NR^{1c};
-(3-11 membered heterocyclyl optionally substituted with 0-6 R^{1a} and/or R^{1b} groups)-(CR^{1a}R^{1b})₁₋₅-A- wherein A is O, S, or NR^{1c};-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c};
-(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b}groups)-(CR^{1a}R^{1b})₁₋₅-A- wherein A is O, S, or NR^{1c};
-(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c};
-(3-11 membered heterocyclyl optionally substituted with 0-6 R^{1a} and/or R^{1b} groups)-A-(CR^{1a}R^{1b})₁₋₅-A- wherein each A is independently O, S, or NR^{1c};
-(3-11 membered heterocyclyl optionally substituted with 0-6 R^{1a} and/or R^{1b} groups)-(CO)-A-(CR^{1a}R^{1b})₁₋₅— wherein A is O, S, or NR^{1c};
-(heteroaryl optionally substituted with 0-4 R^{1a} and/or R^{1b} groups)-A-(CR^{1a}R^{1b})₁₋₅— wherein A is O, S, or NR^{1c};
-(3-11 membered cycloalkyl optionally substituted with 0-6 R^{1a} and/or R^{1b} groups)-(CO)—(CR^{1a}R^{1b})₁₋₅-A- wherein A is O, S, or NR^{1c};
-(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or
—(CO)-(3-11 membered cycloalkyl optionally substituted with 0-6 R^{1a} and/or R^{1b}groups)-(CR^{1a}R^{1b})₁₋₅-A- wherein A is O, S, or NR^{1c}.

In some embodiments of the degrader compounds of formula IA- 11-VHL or formula IA-12-VHL, each R^{1a}, each R^{1b}, and each R^{1c} is independently H or C₁-C₆alkyl. In some embodiments of the degrader compound of formula IA- 11-VHL or formula IA-12-VHL, R¹ is —(CR^{1a}R^{1b})₁₋₅, such as, for example, —CH₂—, —CH₂CH₂—, —CH₂CH₂CH₂—, —CH₂CH₂CH₂CH₂—, —CH₂CH₂CH₂CH₂CH₂—, and the like.

In some embodiments of the degrader compound of formula IA- 11-VHL or formula IA-12-VHL, R¹ is —(CR^{1a}R^{1b})₁₋₅-A- wherein A is O, S, or NR^{1c}, such as, for example, —CH₂—O—, —CH₂CH₂—O—, —CH₂CH₂CH₂—O—, and the like. In some embodiments of the compounds of formula I, R¹ is —(CR^{1a}R^{1b})₁₋₅-A-(CR^{1a}R^{1b})₁₋₅— wherein A is O, S, or NR^{1c}, such as, for example, —CH₂CH₂CH₂—N(CH₃)—CH₂CH₂—, —CH₂CH₂—N(CH₃)—CH₂CH₂—, —CH₂CH₂—O—CH₂—, and the like.

In some embodiments of the degrader compounds of formula I, R¹ is -(3-11 membered heterocyclyl optionally substituted with 0-6 R^{1a} and/or R^{1b} groups)-(CO)—(CR^{1a}R^{1b})₁₋₅-A- wherein A is O, S, or NR^{1c}, such as, for example, -piperidinyl-(CO)—CH(CH₃)—O—, -pyrrolidinyl-(CO)—CH(CH₃)—O—, -piperidinyl-(CO)—CH₂—O—, -methylpiperidinyl-(CO)—CH₂—O—, and the like. In some embodiments of the degrader compounds of formula I, R¹ is -(3-11 membered heterocyclyl optionally substituted with 0-6 R^{1a} and/or R^{1b} groups)-(CO)-A-(CR^{1a}R^{1b})₁₋₅— wherein A is O, S, or NR^{1c}, such as, for example, -piperidinyl-(CO)—O—CH₂—, and the like.

In some embodiments of the degrader compounds of formula I, R¹ is -(3-11 membered heterocyclyl optionally substituted with 0-6 R^{1a} and/or R^{1b} groups)-(CR^{1a}R^{1b})₁₋₅-A- wherein A is O, S, or NR^{1c}, such as, for example, -azabicyclo[3.1.1]heptanyl-CH₂CH₂—O—, -azaspiro[3.3]heptanyl-CH₂CH₂—O—, -piperidinyl-CH₂CH₂—O—, -fluoropiperidinyl-CH₂CH₂—O—, -azepanyl-CH₂CH₂—O—, -pyrrolidinyl-CH₂CH₂—O—, -piperidinyl-CH₂CH(CH₃)—O—, -octahydrocyclopenta[c]pyrrolyl-CH₂CH₂—O—, -pyrrolidinyl-CH₂CH(CH₃)—O—, -methylpiperidinyl-CH₂CH₂—O—, -piperidinyl-CH₂CH(CH₂CH₃)—O—, -pyrrolidinyl-CH₂CH(CH₂CH₃)—O—, -pyrrolidinyl-CH₂CH(CH₃)—O—, -hydroxypyrrolidinyl-CH₂CH₂—O—, -hydroxypiperidinyl-CH₂CH₂—O—, and the like.

In some embodiments of the degrader compounds of formula I, R¹ is -(3-11 membered heterocyclyl optionally substituted with 0-6 R^{1a} and/or R^{1b} groups)-(CR^{1a}R^{1b})₁₋₅—, such as, for example, -piperidinyl-CH₂—, -piperidinyl-CH₂CH₂—, -piperidinyl-CH₂CH₂CH₂—, azetidinyl-CH₂CH₂CH₂—, -aziridinyl-CH₂—, -pyrrolidinyl-CH₂CH₂—, and the like.

In some embodiments of the degrader compounds of formula I, R¹ is -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c}, such as, for example, -piperidinyl-pyrrolidinyl-CH₂CH₂—O—, -piperidinyl- piperidinyl-CH₂CH₂—O—, -pyrrolidinyl-piperidinyl-CH₂CH₂—O—, and the like.

In some embodiments of the degrader compounds of formula I, R¹ is -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, such as, for example, -piperidinyl-CH₂-piperidinyl-CH₂CH₂—O—, -piperidinyl-CH₂CH₂-piperidinyl-CH₂CH₂—O—, and the like.

In some embodiments of the degrader compounds of formula I, R¹ is -(3-11 membered cycloalkyl optionally substituted with 0-6 R^{1a} and/or R^{1b} groups)-A-(CR^{1a}R^{1b})₁₋₅— wherein each A is independently O, S, or NR^{1c}, such as, for example, -cyclohexyl-N(CH₃)—CH₂CH₂—O—, and the like.

In some embodiments of the degrader compounds of formula I, R¹ is —(CO)-(3-11 membered heterocyclyl optionally substituted with 0-6 R^{1a} and/or R^{1b} groups)-(CR^{1a}R^{1b})₁₋₅-A- wherein A is O, S, or NR^{1c}, such as, for example, —(CO)-piperidinyl-CH₂CH₂—O—, and the like.

In some embodiments of the degrader compounds of formula I, R^{j} is —(CR^{1a}R^{1b})₁₋₅-(3-11 membered heterocyclyl optionally substituted with 0-6 R^{1a} and/or R^{1b} groups)-(CR^{1a}R^{1b})₁₋₅₋, such as, for example, —CH₂-piperidinyl-CH₂—, —CH₂-piperidinyl-CH₂CH₂—, —CH₂-piperidinyl-CH₂CH₂CH₂—, and the like.

In some embodiments of the degrader compounds of formula I, R^{j} is —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-, wherein each A is independently O, S, or NR, such as, for example, —CH₂-pyrrolidinyl-CH₂CH₂—O—, —CH₂-pyridinyl-CH₂CH(CH₃)—O—, —CH₂-pyridinyl-CH₂CH₂—O—, —CH(CH₃)-pyridinyl-CH₂CH₂—O—, —CH₂-azepanyl-CH₂CH(CH₃)—O—, —CH₂-azabicyclo[3.2.1]octanyl-CH₂CH₂—O—, —CH₂-(dimethyl)piperidinyl-CH₂CH₂—O—, —CH₂dihydropiperidinyl-CH₂CH₂—O—, and the like.

In some embodiments of the degrader compounds of formula I, R¹ is —(CR^{1a}R^{1b})₁₋₅-(3-11 membered heterocyclyl optionally substituted with 0-6 R^{1a} and/or R^{1b} groups)-A- wherein A is O, S, or NR^{1c}, such as, for example, —CH₂CH₂CH₂-pyrrolidinyl-O—, and the like. In some embodiments of the degrader compounds of formula I, R¹ is -(heteroaryl optionally substituted with 0-4 R^{1a} and/or R^{1b} groups)-A-(CR^{1a}R^{1b})₁₋₅— wherein A is O, S, or NR^{1c}, such as, for example, -pyridinyl-O—CH₂—, and the like. In some embodiments of the degrader compounds of formula I, R¹ is -A-(3-11 membered heterocyclyl optionally substituted with 0-6 R^{1a} and/or R^{1b}groups)-(CR^{1a}R^{1b})₁₋₅-A- wherein each A is independently O, S, or NR^{1c}, such as, for example, —N(CH₃)-piperidinyl-CH₂CH₂—O—, and the like.

In some aspects, the degrader compounds of Formula I are those having the formula IA-13a-VHL, IA-13b-VHL, IA-14a-VHL or IA-14b-VHL:

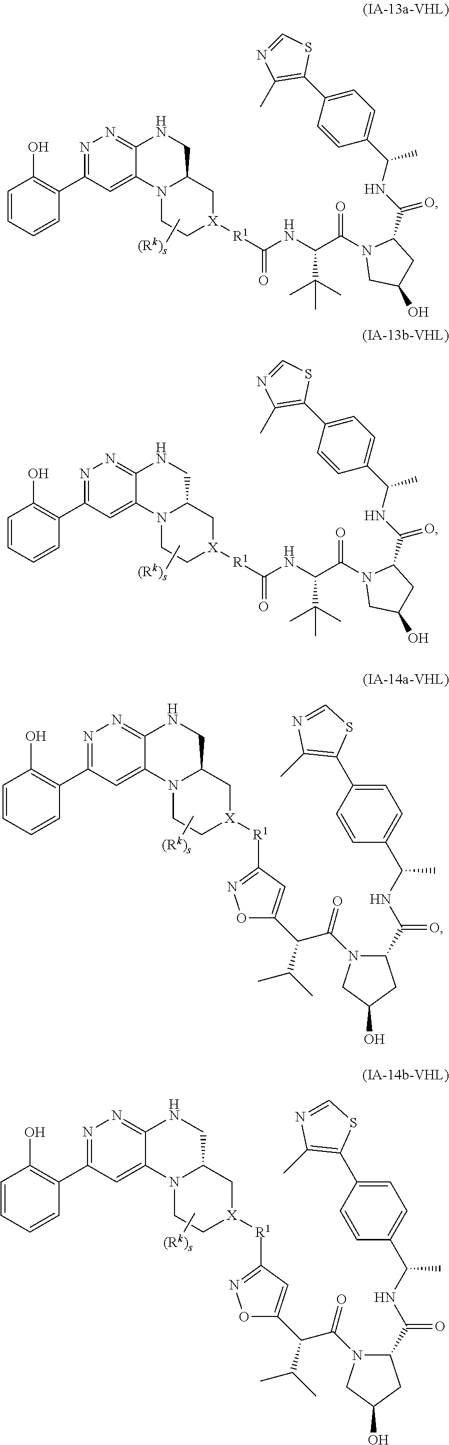

wherein X is N or CH.

In some embodiments of the degrader compounds of formula IA-13a-VHL, IA-13b-VHL, IA-14a-VHL or IA-14b-VHL, R¹ is -(3-11 membered heterocyclyl optionally substituted with 0-6 R^{1a} and/or R^{1b} groups)-(CO)—(CR^{1a}R^{1b})₁₋₅-A- wherein A is O, S, or NR^{1c}, or -(3-11 membered heterocyclyl optionally substituted with 0-6 R^{1a} and/or R^{1b} groups)-(CR^{1a}R^{1b})₁.s-A- wherein A is O, S, or NR^{1c}, R^{1a}, R^{1b}, R^{1c}, and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1c} groups.

In some embodiments of the degrader compounds of formula IA-13a-VHL, IA-13b-VHL, IA-14a-VHL or IA-14b-VHL, R¹ is-(3-11 membered heterocyclyl)-(CO)—(CR^{1a}R^{1b})₁₋₃—O— wherein each R^{1a} is H, C₁₋₃ alkyl, or halo, and each R^{1b} is independently H or —C₁-C₈alkyl, preferably —CH₃.

In other embodiments of the degrader compounds of formula IA-13a-VHL, IA-13b-VHL-VHL, IA-14a-VHL or IA-14b-VHL, R¹ is -(3-11 membered heterocyclyl)-(CR^{1a}R^{1b})₁₋₃—O—, wherein each R^{1a} is H, C₁₋₃ alkyl, or halo, and each R^{1b} is independently H or —C₁-C₈alkyl, preferably —CH₃.

In other embodiments of the degrader compounds of formula IA-13a-VHL, IA-13b-VHL, IA-14a-VHL or IA-14b-VHL, R¹ is -azabicyclo[3.1.1]heptanyl-CH₂CH₂—O—, -azaspiro[3.3]heptanyl-CH₂CH₂—O—, -piperidinyl-CH₂CH₂—O—, -fluoropiperidinyl-CH₂CH₂—O—, -azepanyl-CH₂CH₂—O—, -pyrrolidinyl-CH₂CH₂—O—, -piperidinyl-CH₂CH(CH₃)—O—, -octahydrocyclopenta[c]pyrrolyl-CH₂CH₂—O—, -pyrrolidinyl-CH₂CH(CH₃)—O—, -methylpiperidinyl-CH₂CH₂—O—, -piperidinyl-CH₂CH(CH₂CH₃)—O—, -pyrrolidinyl-CH₂CH(CH₂CH₃)—O—, -pyrrolidinyl-CH₂CH(CH₃)—O—, -hydroxypyrrolidinyl-CH₂CH₂—O—, -hydroxypiperidinyl-CH₂CH₂—O—, and the like.

In some aspects, the degrader compounds of Formula I are those having the formula IA-15a-VHL, IA-15b-VHL, IA-16a-VHL, or IA-16b-VHL:

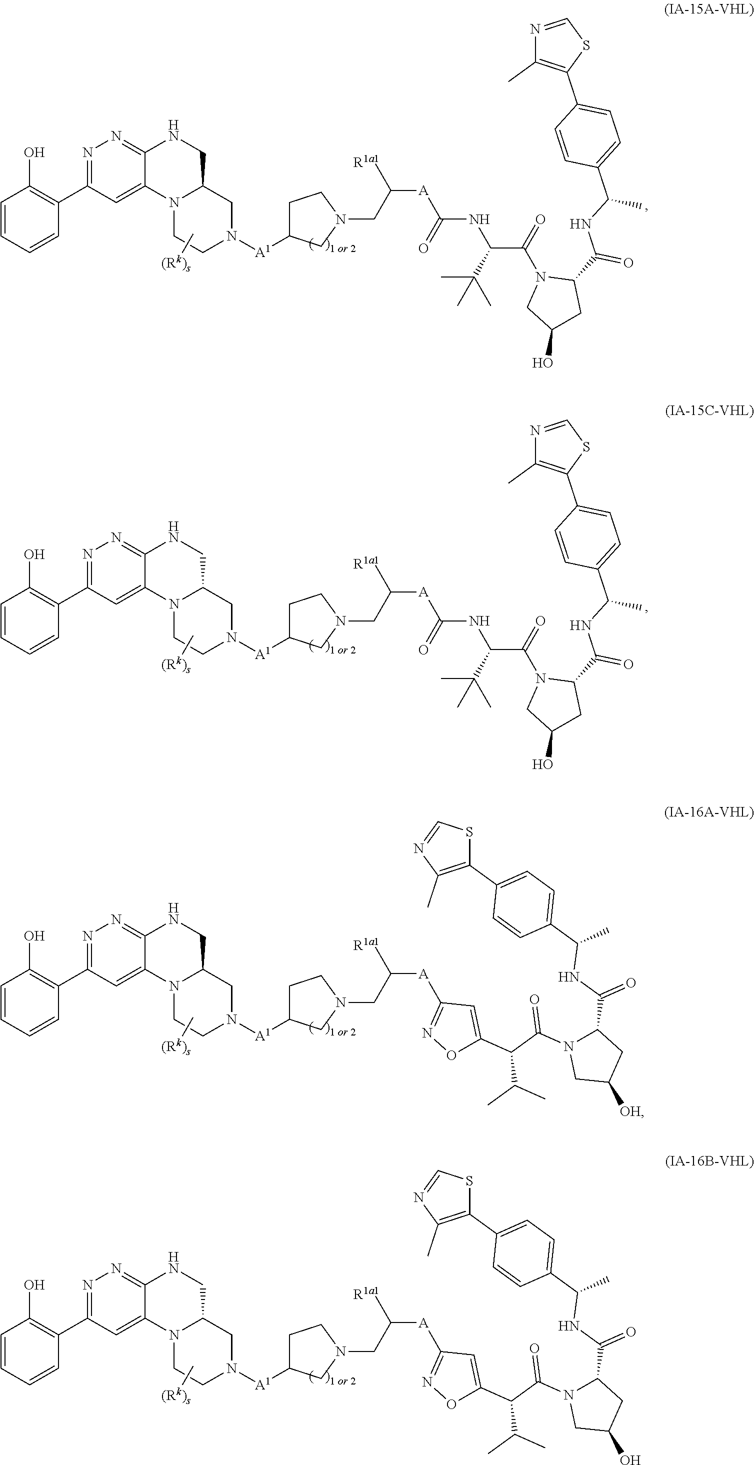

wherein A is O, S, or NR^{1c},
R^{1a1} is H or —C₁-C₈alkyl, preferably —CH₂CH₃, or —CH₃;
   R^{1c} is —H, or —C₁-C₈alkyl, preferably —CH₃;
   A¹ is a covalent bond or —(CR^{1a}R^{1b})₁₋₃; and
   each R^{k} is independently H, deuterium, F, C₁₋₃ alkyl, C₁₋₃ haloalkyl, C₁₋₄ alkoxyl, substituted C₁₋₃ alkyl, substituted C₁.3 haloalkyl, or substituted C₁₋₄ alkoxyl; and s=0-7.

In some embodiments of the degrader compound of formula IA-15a-VHL, IA-15b-VHL, IA-16a-VHL, or IA-16b-VHL, A is O and R^{1a1} is —C₁-C₈alkyl, preferably —CH₂CH₃, or —CH₃. In some embodiments of the degrader compound of formula IA-15a-VHL, IA-15b-VHL, IA-16a-VHL, or IA-16b-VHL, A is O and R^{1a1} is —CH₃.

In some embodiments of the degrader compound of formula IA-15a-VHL, IA-15b-VHL, IA-16a-VHL, or IA-16b-VHL, A¹ is a covalent bond. In some embodiments of the degrader compound of formula IA-15a-VHL, IA-15b-VHL, IA-16a-VHL, or IA-16b-VHL, A¹ is —(CR^{1a}R^{1b})₁₋₃.

In some embodiments of the degrader compound of formula IA-15a-VHL, IA-15b-VHL, IA-16a-VHL, or IA-16b-VHL, s=0. In some embodiments of the degrader compound of formula IA-15a-VHL, IA-15b-VHL, IA-16a-VHL, or IA-16b-VHL, s=1 and R^{k} is —CH₃.

In some embodiments, the degrader compound comprises a small molecule E3 Ubiquitin Ligase binding moiety that binds a Von Hippel-Lindau E3 Ubiquitin Ligase (VHL). In some embodiments, the degrader compound is:
(2S,4R)-4-hydroxy-1-((S)-2-(2-((R)-2-(2-hydroxyphenyl)-5,6,6a,7,9,10-hexahydro-8H-pyrazino[1′,2′:4,5]pyrazino[2,3-c]pyridazin-8-yl)acetamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((S)-2-(2-((S)-2-(2-hydroxyphenyl)-5,6,6a,7,9,10-hexahydro-8H-pyrazino[1′,2′:4,5]pyrazino[2,3-c]pyridazin-8-yl)acetamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((S)-2-(2-((S)-2-(2-hydroxyphenyl)-5,6,6a,7,9,10-hexahydro-8H-pyrazino[1′,2′:4,5]pyrazino[2,3-c]pyridazin-8-yl)acetamido)-3,3-dimethylbutanoyl)-N-(4-(5-methylthiazol-4-yl)benzyl)pyrrolidine-2-carboxamide;
(2S,4R)-4-hydroxy-1-((S)-2-(2-((R)-2-(2-hydroxyphenyl)-5,6,6a,7,9,10-hexahydro-8H-pyrazino[1′,2′:4,5]pyrazino[2,3-c]pyridazin-8-yl)acetamido)-3,3-dimethylbutanoyl)-N-(4-(5-methylthiazol-4-yl)benzyl)pyrrolidine-2-carboxamide;
(2S,4R)-4-hydroxy-1-((S)-2-(2-(4-(((S)-2-(2-hydroxyphenyl)-5,6,6a,7,9,10-hexahydro-8H-pyrazino[1′,2′:4,5]pyrazino[2,3-c]pyridazin-8-yl)methyl)-4-methylpiperidin-1-yl)acetamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((S)-2-(2-(4-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)acetamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-2-(2-hydroxyphenyl)-5,6,6a,7,9,10-hexahydro-8H-pyrazino[1′,2′:4,5]pyrazino[2,3-c]pyridazin-8-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-2-(2-hydroxyphenyl)-5,6,6a,7,9,10-hexahydro-8H-pyrazino[1′,2′:4,5]pyrazino[2,3-c]pyridazin-8-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((S)-2-(3-(2-(4-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((S)-2-(3-(2-(4-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((S)-2-(4-((R)-2-(2-hydroxyphenyl)-5,6,6a,7,9,10-hexahydro-8H-pyrazino[1′,2′:4,5]pyrazino[2,3-c]pyridazin-8-yl)butanamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((S)-2-(4-((S)-2-(2-hydroxyphenyl)-5,6,6a,7,9,10-hexahydro-8H-pyrazino[1′,2′:4,5]pyrazino[2,3-c]pyridazin-8-yl)butanamido)-3,3-dimethylbutanoyl)-N—((S)-1-(4-(4-methylthiazol-5-yl)phenyl)ethyl)pyrrolidine-2-carboxamide;
2-((S)-2-(2-hydroxyphenyl)-5,6,6a,7,9,10-hexahydro-8H-pyrazino[1′,2′:4,5]pyrazino[2,3-c]pyridazin-8-yl)ethyl ((R)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamate;
2-((R)-2-(2-hydroxyphenyl)-5,6,6a,7,9,10-hexahydro-8H-pyrazino[1′,2′:4,5]pyrazino[2,3-c]pyridazin-8-yl)ethyl ((R)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamate;
(2S,4R)-4-hydroxy-1-((S)-2-(3-(4-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propanamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((S)-2-(3-(4-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propanamido)-3,3-dimethylbutanoyl)-N—((S)-1-(4-(4-methylthiazol-5-yl)phenyl)ethyl)pyrrolidine-2-carboxamide;
(3R,5S)-1-((R)-2-(3-(2-((S)-2-(2-hydroxyphenyl)-5,6,6a,7,9,10-hexahydro-8H-pyrazino[1′,2′:4,5]pyrazino[2,3-c]pyridazin-8-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl acetate;
(3R,5S)-1-((R)-2-(3-(2-((R)-2-(2-hydroxyphenyl)-5,6,6a,7,9,10-hexahydro-8H-pyrazino[1′,2′:4,5]pyrazino[2,3-c]pyridazin-8-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl acetate;(3R,5S)-1-((R)-2-(3-(2-((S)-2-(2-hydroxyphenyl)-5,6,6a,7,9,10-hexahydro-8H-pyrazino[1′,2′:4,5]pyrazino[2,3-c]pyridazin-8-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl isobutyrate;
(3R,5S)-1-((R)-2-(3-(2-((R)-2-(2-hydroxyphenyl)-5,6,6a,7,9,10-hexahydro-8H-pyrazino[1′,2′:4,5]pyrazino[2,3-c]pyridazin-8-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl isobutyrate;
(2S,4R)-4-hydroxy-1-((S)-2-(2-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)acetamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((S)-2-(2-(4-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)acetamido)-3,3-dimethylbutanoyl)-N—((S)-1-(4-(4-methylthiazol-5-yl)phenyl)ethyl)pyrrolidine-2-carboxamide;
(2S,4R)-4-hydroxy-1-((2R)-2-(3-(2-(6-((S)-2-(2-hydroxyphenyl)-5,6,6a,7,9,10-hexahydro-8H-pyrazino[1′,2′:4,5]pyrazino[2,3-c]pyridazin-8-yl)-3-azabicyclo[3.1.1]heptan-3-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((2R)-2-(3-(2-(6-((R)-2-(2-hydroxyphenyl)-5,6,6a,7,9,10-hexahydro-8H-pyrazino[1′,2′:4,5]pyrazino[2,3-c]pyridazin-8-yl)-3-azabicyclo[3.1.1]heptan-3-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(6-((S)-2-(2-hydroxyphenyl)-5,6,6a,7,9,10-hexahydro-8H-pyrazino[1′,2′:4,5]pyrazino[2,3-c]pyridazin-8-yl)-2-azaspiro[3.3]heptan-2-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(6-((R)-2-(2-hydroxyphenyl)-5,6,6a,7,9,10-hexahydro-8H-pyrazino[1′,2′:4,5]pyrazino[2,3-c]pyridazin-8-yl)-2-azaspiro[3.3]heptan-2-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-(4-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((S)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((S)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((R)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((R)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((S)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((S)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((R)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((R)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-(4-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-(4-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-(4-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-(4-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-(4-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4R)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4S)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4R)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4S)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4R)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4R)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4S)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4S)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)-2-oxoethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-((S)-2-(2-hydroxyphenyl)-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-((R)-2-(2-hydroxyphenyl)-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((R)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((S)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((S)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((S)-1-((R)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((R)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((S)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((S)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(((R)-1-((R)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)propan-2-yl)oxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2R,4R)-4-((S)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2S,4R)-4-((S)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2R,4S)-4-((S)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2S,4S)-4-((S)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2R,4R)-4-((R)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2S,4R)-4-((R)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2R,4S)-4-((R)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((2S,4S)-4-((R)-2-(2-hydroxyphenyl)-5,6,6a,7,9,10-hexahydro-8H-pyrazino[1′,2′:4,5]pyrazino[2,3-c]pyridazin-8-yl)-2-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4S)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4R)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4R)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4S)-4-((S)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4S)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4R)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3R,4R)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((3 S,4S)-4-((R)-2-(2-hydroxyphenyl)-5,6,6a,7,9,10-hexahydro-8H-pyrazino[1′,2′:4,5]pyrazino[2,3-c]pyridazin-8-yl)-3-methylpiperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((S)-1-((S)-2-(2-hydroxyphenyl)-5,6,6a,7,9,10-hexahydro-8H-pyrazino[1′,2′:4,5]pyrazino[2,3-c]pyridazin-8-yl)ethyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((R)-1-((S)-2-(2-hydroxyphenyl)-5,6,6a,7,9,10-hexahydro-8H-pyrazino[1′,2′:4,5]pyrazino[2,3-c]pyridazin-8-yl)ethyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((S)-1-((R)-2-(2-hydroxyphenyl)-5,6,6a,7,9,10-hexahydro-8H-pyrazino[1′,2′:4,5]pyrazino[2,3-c]pyridazin-8-yl)ethyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((R)-1-((R)-2-(2-hydroxyphenyl)-5,6,6a,7,9,10-hexahydro-8H-pyrazino[1′,2′:4,5]pyrazino[2,3-c]pyridazin-8-yl)ethyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((R)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((S)-3-((S)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((S)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((R)-3-((R)-2-(2-hydroxyphenyl)-5,6,6a,7,9,10-hexahydro-8H-pyrazino[1′,2′:4,5]pyrazino[2,3-c]pyridazin-8-yl)pyrrolidin-1-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((R)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-((S)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(3-((S)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propyl)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(3-((R)-3-(((S)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propyl)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(3-((R)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propyl)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(3-((S)-3-(((R)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)propyl)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((6R,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide; or
(2S,4R)-4-hydroxy-1-((R)-2-(3-(2-(4-((6S,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide.

### Cereblon E3 Ubiquitin Ligase (CRBN)

In some aspects, the ULM moiety in the degrader compounds of the disclosure is a small molecule E3 Ubiquitin Ligase binding moiety that binds a Cereblon E3 Ubiquitin Ligase (CRBN). Such ULM moieties that bind to CRBN are known to those of skill in the art. Methods of determining whether a small molecule binds a Cereblon E3 Ubiquitin Ligase are known in the art, for example, see Lai A-C., Crews C. M. Nat Rev Drug Discov. 2017; 16(2):101-114.

In some embodiments, the ULM is a previously described ULM.

In some embodiments, the ULM is a ULM moiety described in WO 2020/010227, the entirety of which is incorporated by reference herein. In other embodiments, the ULM is a ULM moiety described in WO 2020/081450, the entirety of which is incorporated by reference herein.

In other embodiments, the ULM is a ULM moiety described in WO 2018/102725, the entirety of which is incorporated by reference herein.

In some embodiments, the ULM is a moiety having the Formula ULM-I

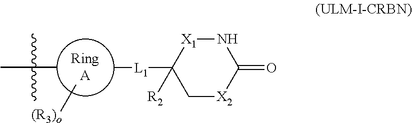

wherein:
[symbol] is a point of attachment to R¹ of PTM Formula IA;
Ring A is a monocyclic, bicyclic or tricyclic aryl, heteroaryl or heterocycloalkyl group,
L₁ is a bond, —O—, —S—, —NR^{a}—, —C(R^{a})₂—, or —C(O)NR^{a}—;
X₁ is a bond, —C(O)—, —C(S)—, —CH₂—, —CHCF₃—, SO₂—, —S(O), P(O)R^{b}—or —P(O)OR^{b}—;
X₂ is —C(R^{a})₂—, —NR^{a}— or —S—;
R₂ is H, deuterium, optionally substituted C₁₋₄ alkyl, C₁₋₄ alkoxyl, C₁₋₄ haloalkyl, —CN, —OR^{a}, —OR^{b} or —SR^{b};
each R₃ is independently H, deuterium, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c2}R^{d}, —NR^{a}R^{c2}, —C(O)R^{b}, —OC(O)R^{a}, —C(O)OR^{a}, —C(O)NR^{c2}R^{d}, —S(O)R^{b}, —S(O)₂NR^{c2}R^{d}, —S(O)(═NR^{b})R^{b}, —SF₅, —P(O)R^{b}R^{b}, —P(O)(OR^{b})(OR^{b}), —B(OR^{d})(OR^{c2}) or —S(O)₂R^{b};
each R^{a} is independently H, deuterium, —C(O)R^{b}, —C(O)OR^{c2}, —C(O)NR^{c2}R^{d}, —C(═NR^{b})NR^{b}R^{c2}, —C(═NOR^{b})NR^{b}R^{c2}, —C(═NCN)NR^{b}R^{c2}, —P(OR^{c2})₂, —P(O)R^{c2}R^{b}, —P(O)OR^{c2}OR^{b}, —S(O)R^{b}, —S(O)NR^{c2}R^{d}, —S(O)₂R^{b}, —S(O)₂NR^{c2}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, deuterium, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c2} or R^{d} is independently H, deuterium, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c2} and R^{d}, together with the atom to which they are both attached, form a monocyclic or multicyclic heterocycloalkyl, or a monocyclic or multicyclic heterocyclo-alkenyl group;
o is 1, 2, 3, 4, or 5.

In some embodiments of ULM-I-CRBN, Ring A is a bicyclic or tricyclic heteroaryl or heterocycloalkyl group. In some embodiments of ULM-1, Ring A is heteroaryl bicyclic. In some embodiments of ULM-1, Ring A is heteroaryl tricyclic. In some embodiments of ULM-1, Ring A is heterobicycloalkyl. In some embodiments of ULM-1, Ring A is heterotricycloalkyl.

In other embodiments of ULM-I-CRBN, Ring A is a monocyclic heteroaryl having at least one N atom. In other embodiments of ULM-I-CRBN, Ring A is a pyridine or a pyridazine. In other embodiments of ULM-I-CRBN, Ring A is

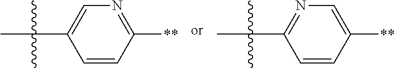

or wherein [symbol] is a point of attachment to PTM and ** is a point of attachment to L₁.

In yet other embodiments, Ring A is

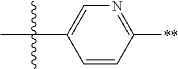

wherein [symbol] is a point of attachment to PTM and ** is a point of attachment to L₁. In yet other embodiments, Ring A is

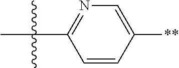

wherein [symbol] is a point of attachment to PTM and ** is a point of attachment to L₁.

In other embodiments of ULM-I-CRBN, Ring A is a bicyclic heteroaryl having at least one N atom. In other embodiments of ULM-I-CRBN, Ring A is an isoindolin-one, an isoindolin-dione, an isoquinolin-one or an isoquinolin-dione. In other embodiments of ULM-I-CRBN, Ring A is or

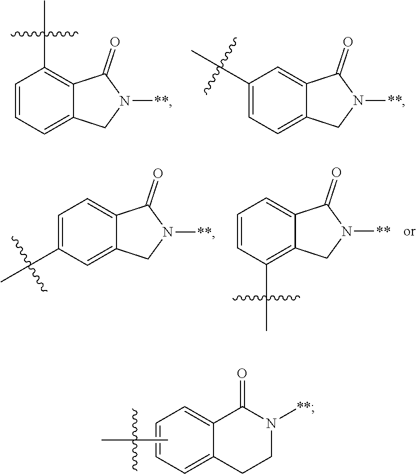

wherein [symbol] is a point of attachment to PTM and ** is a point of attachment to L₁.

In yet other embodiments, Ring A is

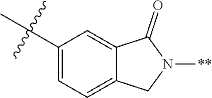

wherein [symbol] is a point of attachment to PTM and ** is a point of attachment to L₁. In yet other embodiments, Ring A is

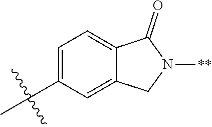

wherein [symbol] is a point of attachment to PTM and ** is a point of attachment to L₁.

In yet other embodiments of ULM-I-CRBN, Ring A is

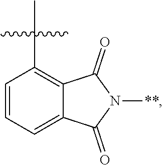

wherein [symbol] is a point of attachment to PTM and ** is a point of attachment to L₁. In yet other embodiments of ULM-I-CRBN, Ring A is

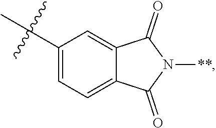

wherein [symbol] is a point of attachment to PTM and ** is a point of attachment to L₁. In yet other embodiments of ULM-I-CRBN, Ring A is

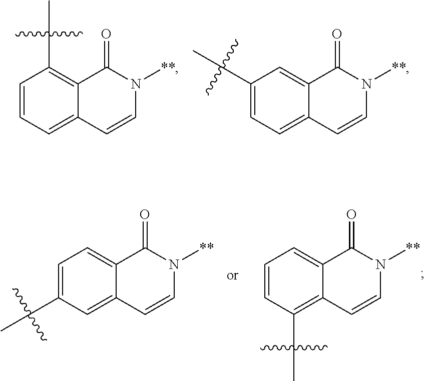

wherein [symbol] is a point of attachment to PTM and ** is a point of attachment to L₁.

In yet other embodiments of ULM-I-CRBN, Ring A is a tricyclic heteroaryl having at least one N atom. In yet other embodiments of ULM-I-CRBN, Ring A is a carbazole, a pyrido-indole or a pyrrolo-dipyridine. In yet other embodiments of ULM-I-CRBN, Ring A is

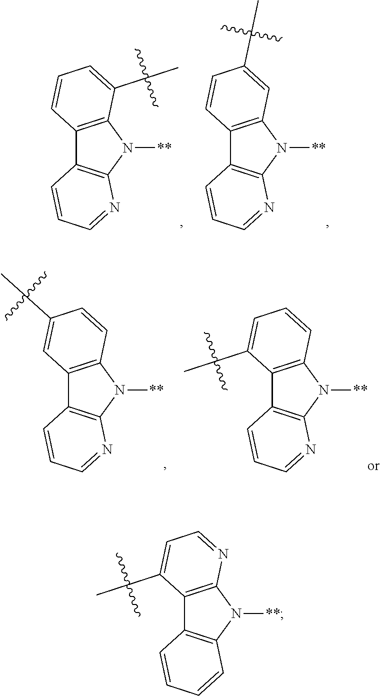

wherein [symbol] is a point of attachment to PTM and ** is a point of attachment to L₁.

In yet other embodiments of ULM-I-CRBN, Ring A is

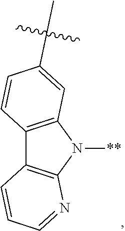

wherein [symbol] is a point of attachment to PTM and ** is a point of attachment to L₁.

In yet other embodiments of ULM-I-CRBN, Ring A is

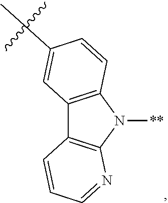

wherein [symbol] is a point of attachment to PTM and ** is a point of attachment to L₁.

In some embodiments of ULM-I-CRBN, L₁ is a bond, —O—, —S—, —NR^{a}—, —C(R^{a})₂—, or —C(O)NR^{a}—. In some embodiments of ULM-I-CRBN, L¹ is a bond. In some embodiments of ULM-I-CRBN, L¹ is C₁-C₆ alkylene. In some embodiments of ULM-I-CRBN, L¹ is —C(O)NR^{a}—.

In some embodiments of ULM-I-CRBN, X₁ is a bond, —C(O)—, —C(S)—, —CH₂—, —CHCF₃—, SO₂—, —S(O), P(O)R^{b}—or —P(O)OR^{b}—. In some embodiments of ULM-I-CRBN, X₁ is a bond. In some embodiments of ULM-I-CRBN, X₁ is —C(O)—. In some embodiments of ULM-I-CRBN, X₁ is —CH₂—. In some embodiments of ULM-I-CRBN, X₁ is —CHCF₃—.

In some embodiments of ULM-I-CRBN, X₂ is —C(R^{a})₂—, —NR^{a}— or —S—. In some embodiments, X₂ is —C(R^{a})₂—.

In some embodiments of ULM-I-CRBN, R₂ is H, deuterium, optionally substituted C₁₋₄ alkyl, C₁₋₄ alkoxyl, C₁₋₄ haloalkyl, —CN, —OR^{a}, —OR^{b} or —SR^{b}. In some embodiments of ULM-I-CRBN, R₂ is H. In some embodiments of ULM-I-CRBN, R₂ is optionally substituted C₁₋₄ alkyl.

In some embodiments of ULM-I-CRBN, each R₃ is independently H, deuterium, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c2}R^{d} —NR^{a}R^{c2}, —C(O)R^{b}, —OC(O)R^{a}, —C(O)OR^{a}, —C(O)NR^{c2}R^{d}, —S(O)R^{b}, —S(O)₂NR^{e2}R^{d}, —S(O)(═NR^{b})R^{b}, —SF₅, —P(O)R^{b}R^{b}, —P(O)(OR^{b})(OR^{b}), —B(OR^{d})(OR^{c2}) or —S(O)₂R^{b}. In some embodiments of ULM-I-CRBN, at least one R₃ is H. In some embodiments of ULM-I-CRBN, each R₃ is H. In some embodiments of ULM-I-CRBN, at least one R₃ is C₁₋₆alkyl.

In some embodiments of ULM-I-CRBN, each R^{a} is independently H, deuterium, —C(O)R^{b}, —C(O)OR^{c2}, —C(O)NR^{c2}R^{d}, —C(═NR^{b})NR^{c2}, —C(═NOR^{b})NR^{c2}, —C(═NCN)NR^{b}R^{c2}, —P(OR^{c2})₂, —P(O)R^{c2}R^{b}, —P(O)OR^{c2}OR^{b}, —S(O)R^{b}, —S(O)NR^{c2}R^{d}, —S(O)₂R^{b}, —S(O)₂NR^{c2}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl. In some embodiments of ULM-I-CRBN, R^{a} is H. In some embodiments, R^{a} is deuterium. In some embodiments, R^{a} is —C(O)R^{b}. In some embodiments, R^{a} is —C(O)OR^{c2}. In some embodiments, R^{a} is —C(O)NR^{c2}R^{d}. In some embodiments, R^{a} is —C(═NR^{b})NR^{b}R^{c2}. In some embodiments, R^{a} is C(═NOR^{b})NR^{b}R^{c2}. In some embodiments, R^{a} is —C(═NCN)NR^{b}R^{c2}. In other embodiments, R^{a} is —P(OR^{c2})₂, —P(O)R^{c2}R^{b}, —P(O)OR^{c2}OR^{b}, —S(O)R^{b}, —S(O)NR^{c2}R^{d}, —S(O)₂R^{b}, —S(O)₂NR^{c2}R^{d}, SiR^{b3}, and the like. In yet other embodiments, R^{a} is —C₁-C₁₀alkyl, —C₂-C₁₀ alkenyl, —C₂-C₁₀ alkynyl, aryl, cycloalkyl, cycloalkenyl, heteroaryl, heterocycloalkyl, heterocycloalkenyl, and the like.

In some embodiments of ULM-I-CRBN, each R^{b}, is independently H, deuterium, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl. In some embodiments, R^{b} is H. In some embodiments, R^{b} is deuterium. In some embodiments, R^{b} is —C₁-C₆ alkyl. In some embodiments, R^{b} is —C₂-C₆ alkenyl. In some embodiments, R^{b} is —C₂-C₆ alkynyl. In other embodiments, R^{b} is aryl. In other embodiments, R^{b} is cycloalkyl. In other embodiments, R^{b} is cycloalkenyl. In other embodiments, R^{b} is heteroaryl. In other embodiments, R^{b} is heterocycloalkyl. In other embodiments, R^{b} is heterocycloalkenyl.

In some embodiments of ULM-I-CRBN, each R^{c2} or R^{d} is independently H, deuterium, —C₁-C₁₀ alkyl, —C₂-C₆ alkenyl, —C₂-C₆ alkynyl, -OC₁-C₆alkyl, —O-cycloalkyl, aryl, heteroaryl, cycloalkyl, cycloalkenyl, heterocycloalkyl, or heterocycloalkenyl. In some embodiments, R^{c2} or R^{d} is H. In some embodiments, R^{c2} or R^{d} is deuterium. In some embodiments, R^{c2} or R^{d} is —C₁-C₁₀ alkyl. In some embodiments, R^{c2} or R^{d} is —C₂-C₆ alkenyl. In some embodiments, R^{c2} or R^{d} is —C₂-C₆ alkynyl. In other embodiments, R^{c2} or R^{d} is -OC1-C₆alkyl. In other embodiments, R^{c2} or R^{d} is —O-cycloalkyl. In other embodiments, R^{c2} or R^{d} is aryl. In other embodiments, R^{c2} or R^{d} is cycloalkyl. In other embodiments, R^{c2} or R^{d} is cycloalkenyl. In other embodiments, R^{c2} or R^{d} is heteroaryl. In other embodiments, R^{c2} or R^{d} is heterocycloalkyl.

In other embodiments of ULM-I-CRBN, R^{c2} and R^{d}, together with the atom to which they are both attached, form a monocyclic or multicyclic heterocycloalkyl, or a monocyclic or multicyclic heterocyclo-alkenyl group. In other embodiments, R^{c2} or R^{d} is heterocycloalkenyl. In yet other embodiments, R^{c2} and R^{d}, together with the atom to which they are both attached, form a monocyclic or multicyclic heterocycloalkyl, or a monocyclic or multicyclic heterocyclo-alkenyl group. In yet other embodiments, R^{c2} and R^{d} form a monocyclic heterocycloalkyl. In yet other embodiments, R^{c2} and R^{d} form a multicyclic heterocycloalkyl. In yet other embodiments, R^{c2} and R^{d} form a monocyclic heterocyclo-alkenyl group. In yet other embodiments, R^{c2} and R^{d}form a multicyclic heterocyclo-alkenyl group.

In some embodiments of ULM-1-CRBN, o is 1, 2, 3, 4 or 5. In some embodiments, o is 1. In some embodiments, o is 2. In other embodiments, o is 3. In other embodiments, o is 4. In yet other embodiments, o is 5.

In some embodiments, ULM-I-CRBN is a compound of formula:

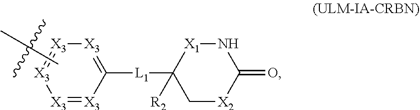

wherein each X₃ is independently N, N-oxide or CR₃ and at least one X₃ is N or N-oxide;
wherein [symbol] is a point of attachment to PTM; or

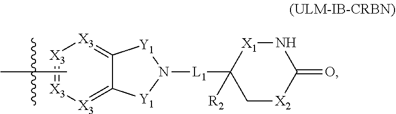

wherein each X₃ is independently N, N-oxide or CR₃;
wherein each Y₁ is independently —C(O)— or —C(R^{a})₂— and at least one Y₁ is —C(O)—; and wherein [symbol] is a point of attachment to PTM; or

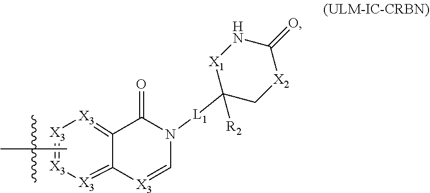

wherein each X₃ is independently N, N-oxide or CR₃ and wherein [symbol] is a point of attachment to PTM; or

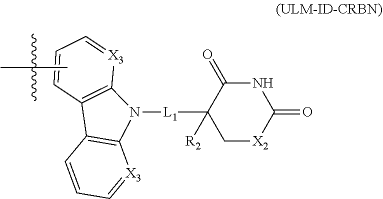

wherein each X₃ is independently N, N-oxide or CR₃ and wherein [symbol] is a point of attachment to PTM.

In some embodiments of ULM-IA-CRBN, ULM-IB-CRBN, ULM-IC-CRBN, or ULM-ID-CRBN, X₂ is —C(R^{a})₂—and R₂ is H.

In some embodiments, the degrader compounds of Formula I are those having the Formula IA-7-CRBN, Formula IA-8-CRBN, Formula IA-9-CRBN, Formula IA-10-CRBN, Formula IA- 11-CRBN, Formula IA-12-CRBN or Formula IA-13-CRBN:

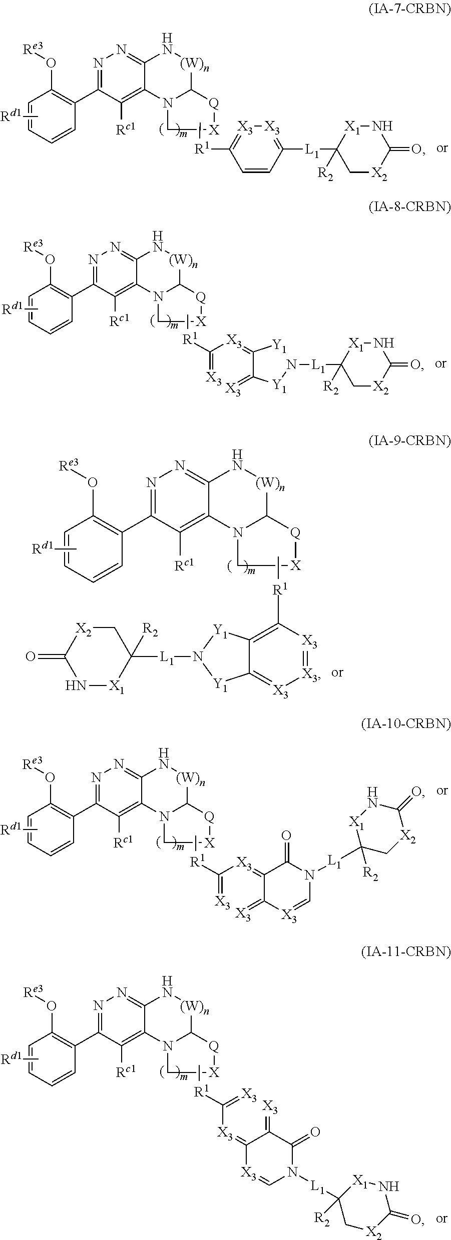

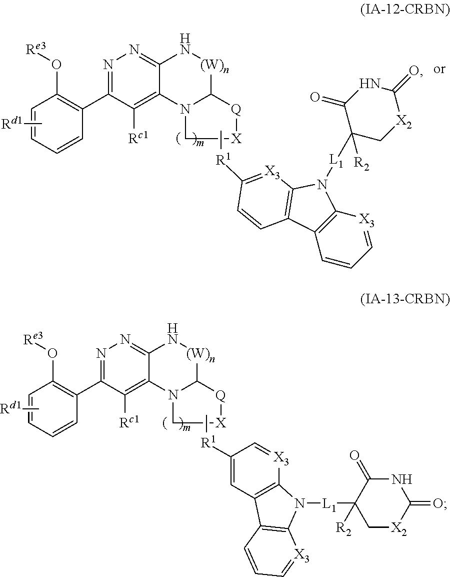

wherein:
each W is independently optionally substituted —CH₂—, —C(O)—, —S(O)—, or —S(O)₂—; wherein when n=2 or 3, only one W may be —C(O)—, —S(O)—, or —S(O)₂—, and the other W are —CH₂— or substituted —CH₂—;
n=0-3;
m=1-3;
X is optionally substituted —CH₂—, or NH; or, if R¹ is attached to X, then X is —CH— or N; Q is optionally substituted —CH₂—, optionally substituted —(CH₂)₂—, —C(O)—, optionally substituted —CH₂C(O)—, —S(O)—, —S(O)₂—, optionally substituted —CH₂S(O)₂—, or optionally substituted —CH₂S(O)—;
R^{c1} and R^{d1} are independently H, deuterium, Halo, C₁₋₃ alkyl, C₁₋₃ haloalkyl, or C₁₋₄ alkoxyl;
R^{e3} is H, —C(O)R, —CH₂—O—P(O)(OR^{g})₂, or —P(O)(OR^{g})₂; wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}—(CR^{1a}R^{1b})₁₋₅-A-(CR^{1a}R^{1b})₁₋₅— wherein A is O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})₁₋₅—(CR^{1a}═CR^{1b})—(CR^{1a}R^{1b})₁₋₅—, —(CR^{1a}R^{1b})₁₋₅₋(CR^{1a}=CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b}groups)-, -(3-11 membered cycloalkyl optionally substituted with 0-6 R^{1a} and/or R^{1b}groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-(3-11 membered heterocyclyl optionally substituted with 0-6 R^{1a} and/or R^{1b} groups)-A- wherein A is O, S, or NR^{1c}, —(CR^{1a}R^{1b})₁₋₅-A-(3-11 membered heterocyclyl optionally substituted with 0-6 R^{1a} and/or R^{1b} groups)- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}—(CR^{1a}R^{1b})₁₋₅-A-(3-11 membered cycloalkyl optionally substituted with 0-6 R^{1a} and/or R^{1b}groups)-(CR^{1a}R^{1b})₁₋₅-A- wherein each A is independently O, S, or NR^{1c}—(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}, —(CR^{1a}R^{1b})₁₋₅-A-(CR^{1a}R^{1b})₁₋₅-A- wherein A is O, S, or NR^{1c}—(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}—(CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}—(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}, —(CR^{1a}R^{1b})₁₋₅-A-(CO)-(3-11 membered heterocyclyl optionally substituted with 0-6 R^{1a} and/or R^{1b}groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}, -(3-11 membered cycloalkyl optionally substituted with 0-6 R^{1a} and/or R^{1b} groups)-(CR^{1a}R^{1b})₁₋₅—, or -(3-11 membered heterocyclyl optionally substituted with 0-6 R^{1a} and/or R^{1b} groups)-(CR^{1a}R^{1b})₁₋₅₋;
L₁ is a bond, —O—, —S—, —NR^{a}—, —C(R^{a})₂—, or —C(O)NR^{a}—;
X₁ is a bond, —C(O)—, —C(S)—, —CH₂—, —CHCF₃—, SO₂—, —S(O), P(O)R^{b}—or —P(O)OR^{b}—;
X₂ is —C(R^{a})₂—, —NR^{a}— or —S—;
R₂ is H, deuterium, optionally substituted C₁₋₄ alkyl, C₁₋₄ alkoxyl, C₁₋₄ haloalkyl, —CN, —OR^{a}, —OR^{b} or —SR^{b};
each X₃ is independently N, N-oxide or CR₃; and
each Y is independently —C(O)— or —C(R^{a})₂— and at least one Y is —C(O)—.

In some embodiments of the degrader compound of Formula IA-7-CRBN, Formula IA-8-CRBN, Formula IA-9-CRBN, Formula IA-10-CRBN, Formula IA-11-CRBN, Formula IA-12-CRBN and Formula IA-13-CRBN, n=1. In other embodiments of the degrader compound of Formula IA-7-CRBN, Formula IA-8-CRBN, Formula IA-9-CRBN, Formula IA-10-CRBN, Formula IA-11-CRBN, Formula IA-12-CRBN and Formula IA-13-CRBN, n=2. In other embodiments of the degrader compound of Formula IA-7-CRBN, Formula IA-8-CRBN, Formula IA-9-CRBN, Formula IA-10-CRBN, Formula IA-11-CRBN, Formula IA-12-CRBN and Formula IA-13-CRBN, n=3.

In some embodiments of the degrader compound of Formula IA-7-CRBN, Formula IA-8-CRBN, Formula IA-9-CRBN, Formula IA-10, Formula IA-11-CRBN, Formula IA-12-CRBN and Formula IA-13-CRBN, m=1. In other embodiments of the degrader compound of Formula IA-7-CRBN, Formula IA-8, Formula IA-9-CRBN, Formula IA-10-CRBN, Formula IA-11-CRBN, Formula IA-12-CRBN and Formula IA-13-CRBN, m=2. In other embodiments of the degrader compound of Formula IA-7-CRBN, Formula IA-8-CRBN, Formula IA-9-CRBN, Formula IA-10-CRBN, Formula IA-11-CRBN, Formula IA-12-CRBN and Formula IA-13-CRBN, m=3.

In some embodiments of the degrader compound of Formula IA-7-CRBN, Formula IA-8-CRBN, Formula IA-9-CRBN, Formula IA-10-CRBN, Formula IA-II-CRBN, Formula IA-12-CRBN and Formula IA-13-CRBN, R^{c1} and R^{d1} are each H.

In some embodiments of the degrader compound of Formula IA-7-CRBN, Formula IA-8-CRBN, Formula IA-9-CRBN, Formula IA-10-CRBN, Formula IA-11-CRBN, Formula IA-12-CRBN and Formula IA-13-CRBN, R^{e3} is H.

In some embodiments of the degrader compound of Formula IA-7-CRBN, Formula IA-8-CRBN, Formula IA-9-CRBN, Formula IA-10-CRBN, Formula IA-11-CRBN, Formula IA-12-CRBN and Formula IA-13-CRBN, R^{c1}, R^{d1}, and R^{e3} are each H.

In some embodiments, the degrader compounds of Formula I are those having the Formula IA-7a-CRBN, Formula IA-8a-CRBN, Formula IA-9a-CRBN, Formula IA-10a-CRBN, Formula IA-11a-CRBN, Formula IA-12a-CRBN or Formula IA-13a-CRBN:

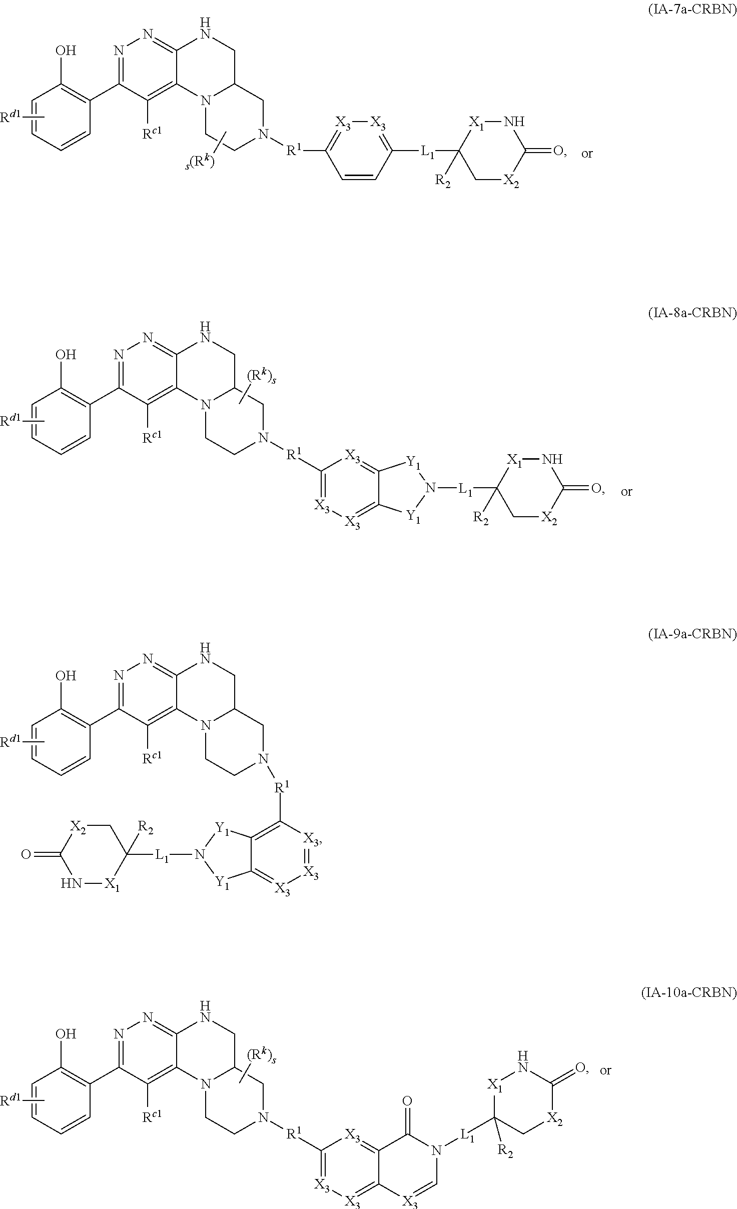

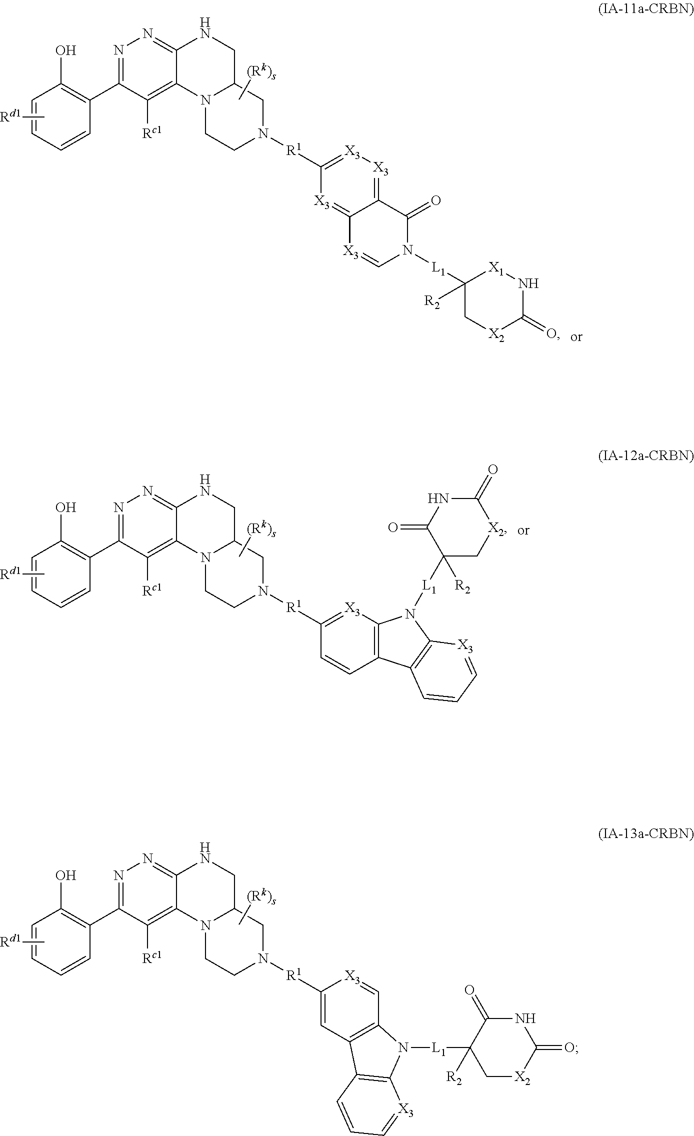

wherein
each R^{k} is independently H, deuterium, F, C₁₋₃ alkyl, C₁₋₃ haloalkyl, C₁₋₄ alkoxyl, substituted C₁₋₃ alkyl, substituted C₁.3 haloalkyl, or substituted C₁₋₄ alkoxyl;
s is 0, 1, 2, 3 or 4;
each Y₁ is independently —C(O)— or —CH₂— and at least one Y₁ is —C(O)—; and
R^{d1}, R^{c1}, R¹, R², X₁, X₂ and X₃ are as defined herein.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, s is 0. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, S is 1. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, S is 2. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, S is 3. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, S is 4.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least one R^{k} is H. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least two R^{k} are H. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, each R^{k} is H.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least one R^{k} is C₁₋₆alkyl. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least two R^{k} are C₁₋₆alkyl. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, each R^{k} is C₁₋₆alkyl.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least one R^{k} is methyl. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least two R^{k} are methyl. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, each R^{k} is methyl.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least one Y₁ is —C(O)—. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, each Y₁ is —C(O)—.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, at least one Y₁ is —CH₂—. In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN, each Y₁ is —CH₂—.

In some embodiments of the degrader compounds of Formula IA-7a-CRBN, Formula IA-8a-CRBN, Formula IA-9a-CRBN, Formula IA-10a-CRBN, Formula IA-11a-CRBN, Formula IA-12a-CRBN and Formula IA-13a-CRBN—CRBN, one Y₁ is —CH₂— and the other Y₁ is —C(O)—.

In some embodiments, the degrader compounds of Formula I are those having the Formula IA-7b-CRBN, Formula IA-8b-CRBN, Formula IA-9b-CRBN, Formula IA-10b-CRBN, Formula IA-11b-CRBN, Formula IA-12b-CRBN or Formula IA-13b-CRBN:

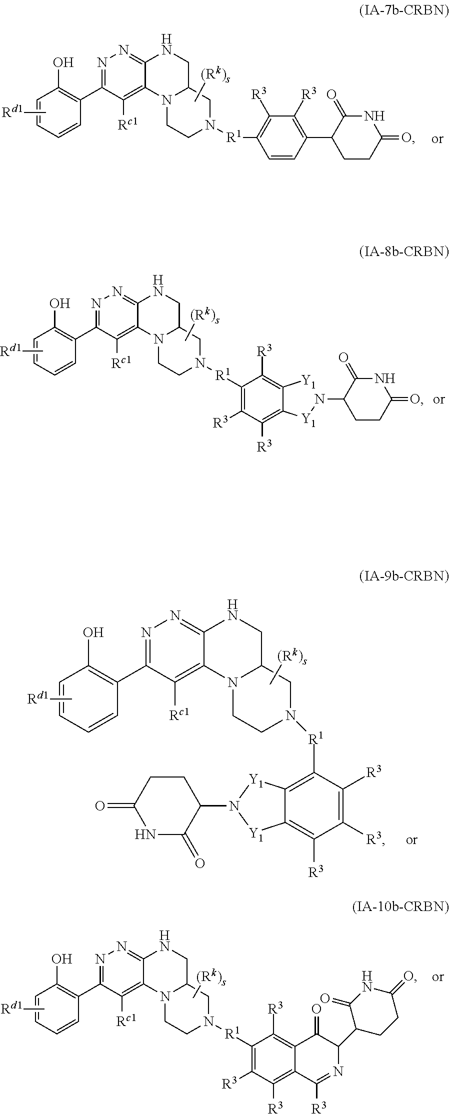

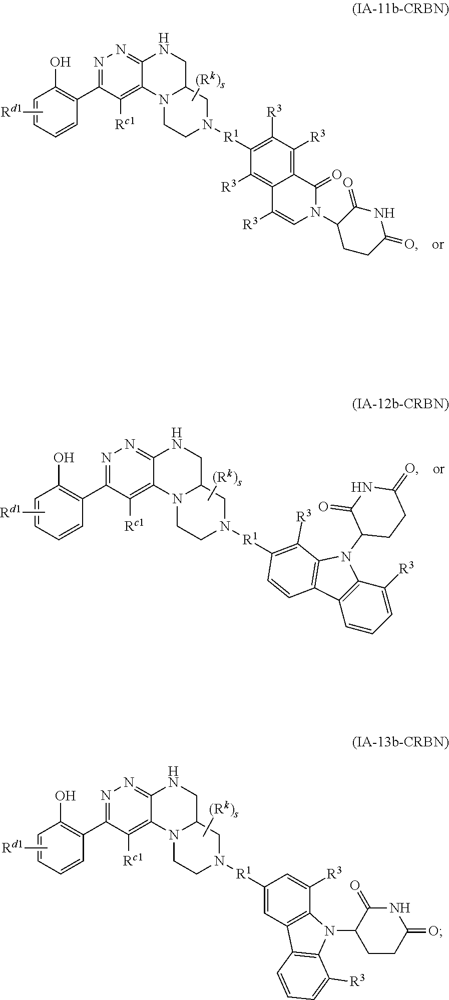

wherein
each R^{k} is independently H, deuterium, F, C₁₋₃ alkyl, C₁₋₃ haloalkyl, C₁₋₄ alkoxyl, substituted C₁₋₃ alkyl, substituted C₁₋₃ haloalkyl, or substituted C₁₋₄ alkoxyl;
s is 0, 1, 2, 3 or 4;
each Y₁ is independently —C(O)— or —CH₂— and at least one Y₁ is —C(O)—; and
R^{d1}, R^{c1}, R¹ and R₃ are as defined herein.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, s is 0. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, s is 1. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, s is 2. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, s is 3. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, s is 4.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least one R^{k} is H. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least two R^{k} are H. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, each R^{k} is H.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least one R^{k} is C₁₋₆alkyl. In some embodiments of the degrader compounds of Formula IA-7b-CRBN, Formula IA-8b-CRBN, Formula IA-9b-CRBN, Formula IA-10b-CRBN, Formula IA-1 b-CRBN, Formula IA-12b-CRBN and Formula IA-13b-CRBN, at least two R^{k} are C₁₋₆alkyl. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, each R^{k} is C₁₋₆alkyl.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least one R^{k} is methyl. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least two R^{k} are methyl. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, each R^{k} is methyl.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least one Y₁ is —C(O)—. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, each Y₁ is —C(O)—.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, at least one Y₁ is —CH₂—. In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, each Y₁ is —CH₂—.

In some embodiments of the degrader compounds of Formula IA-7b-CRBN, Formula IA-8b-CRBN, Formula IA-9b-CRBN, Formula IA-10b-CRBN, Formula IA-11b-CRBN, Formula IA-12b-CRBN and Formula IA-13b-CRBN, one Y₁ is —CH₂— and the other Y₁ is —C(O)—.

In some embodiments, the degrader compounds of Formula I are those having the Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN:

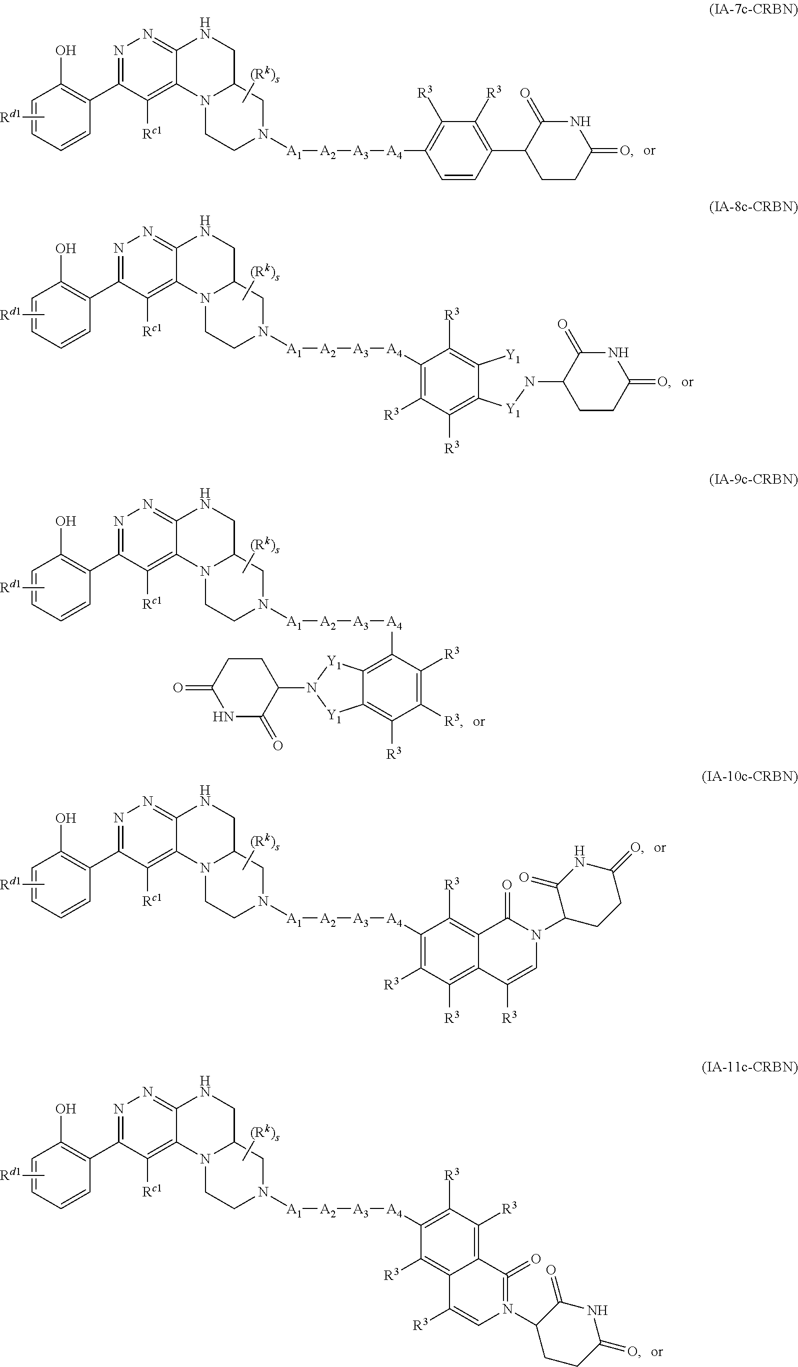

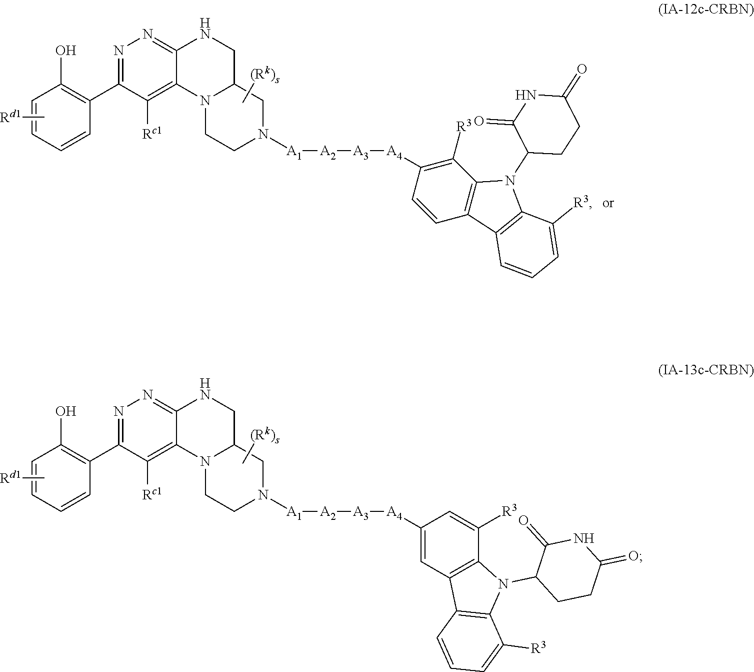

wherein
each R^{k} is independently H, deuterium, F, C₁₋₃ alkyl, C₁₋₃ haloalkyl, C₁₋₄ alkoxyl, substituted C₁₋₃ alkyl, substituted C₁.3 haloalkyl, or substituted C₁₋₄ alkoxyl;
s is 0, 1, 2, 3 or 4;
each Y₁ is independently —C(O)— or —CH₂— and at least one Y₁ is —C(O)—;
A₁ is a bond, —(CR¹R²)ₙ, —C═O, —C(═O)O, —C(═O)NR₃, —SO₂, —SO, aryl, heteroaryl, cycloalkyl, or heterocycloalkyl;
A₂ is a bond, alkyl, cycloalkyl, heteroaryl or heterocycloalkyl;
A₃ is a bond, —(CR¹R²)ₙ, —C═O, —SO₂, SO, aryl, heteroaryl, cycloalkyl or heterocycloalkyl;
A₄ is a bond, alkyl, cycloalkyl, heteroaryl or heterocycloalkyl;
wherein each of A₁, A₂, A₃ and A₄ is optionally substituted with deuterium, halo, alkyl, haloalkyl, —CN, —OR₃, NR^{c2}R^{d}, NO₂, —SR₃, —C═OR^{b}, —C(═O)OR^{b}, —C(═O)NR₃R₃, —SO₂R^{b}, —SOR^{b}, —S(═O)(═NR^{b})N, cycloalkyl or heterocycloalkyl; and
wherein two substituents on each A₁, A₂, A₃, A₄ can be joined to form an additional 3-8 membered ring, such as a spirocycle; and
R^{d1}, R^{c1} and R₃ are as defined herein.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is a bond. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —(CR¹R²)ₙ. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —C═O. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is-C(═O)O. In some embodiments of the degrader compounds of Formula IA-7c-CRBN, Formula IA-8c-CRBN, Formula IA-9c-CRBN, Formula IA-10c-CRBN, Formula IA-11 c-CRBN, Formula IA-12c-CRBN or Formula IA-13c-CRBN, A₁ is —C(═O)NR₃. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —SO₂. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —SO. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is aryl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is heteroaryl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is cycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is heterocycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is optionally substituted with deuterium, halo, alkyl, haloalkyl, —CN, —OR₃, NR^{1c}R^{d}, NO₂, —SR₃, —C═OR^{b}, —C(═O)OR^{b}, —C(═O)NR₃R₃, —SO₂R^{b}, —SOR^{b}, —S(═O)(═NR^{b})N, cycloalkyl or heterocycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a bond. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is alkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is heterocycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is heteroaryl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is cycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is heteroaryl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is optionally substituted with deuterium, halo, alkyl, haloalkyl, —CN, —OR₃, NR^{c2}R^{d}, NO₂, —SR₃, —C═OR^{b}, —C(═O)OR^{b}, —C(═O)NR₃R₃, —SO₂R^{b}, —SOR^{b}, —S(═O)(═NR^{b})N, cycloalkyl or heterocycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is a bond. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is —(CR¹R²)ₙ. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is —C═O. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is —SO₂. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is SO. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is aryl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is heteroaryl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c or Formula IA-13c-CRBN, A₃ is cycloalkyl. In some embodiments of the degrader compounds of Formula IA-7c-CRBN, Formula IA-8c-CRBN, Formula IA-9c-CRBN, Formula IA-10c-CRBN, Formula IA-11 c-CRBN, Formula IA-12c-CRBN or Formula IA-13c-CRBN, A₃ is heterocycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is optionally substituted with deuterium, halo, alkyl, haloalkyl, —CN, —OR₃, NR^{c2}R^{d}, NO₂, —SR₃, —C═OR^{b}, —C(═O)OR^{b}, —C(═O)NR₃R³, —SO₂R^{b}, —SOR^{b}, —S(═O)(═NR^{b})N, cycloalkyl or heterocycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is a bond. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is alkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is heterocycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is heteroaryl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is cycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is heteroaryl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is optionally substituted with deuterium, halo, alkyl, haloalkyl, —CN, —OR₃, NR^{c2}R^{d}, NO₂, —SR₃, —C═OR^{b}, —C(═O)OR^{b}, —C(═O)NR₃R₃, —SO₂R^{b}, —SOR^{b}, —S(═O)(═NR^{b})N, cycloalkyl or heterocycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, two substituents on each A₁, A₂, A₃, A₄ can be joined to form an additional 3-8 membered ring. In some embodiments, the 3-8 membered ring is a spirocycle.

In some embodiments, the degrader compounds of Formula I are those having the Formula IA-7d-CRBN, Formula IA-8d1-CRBN, Formula IA-8d2-CRBN, Formula IA-8d3-CRBN, Formula IA-9d1-CRBN, Formula IA-9d2-CRBN, Formula IA-9d3-CRBN, Formula IA-10d-CRBN, Formula IA-11d-CRBN, Formula IA-12d-CRBN or Formula IA-13d-CRBN:

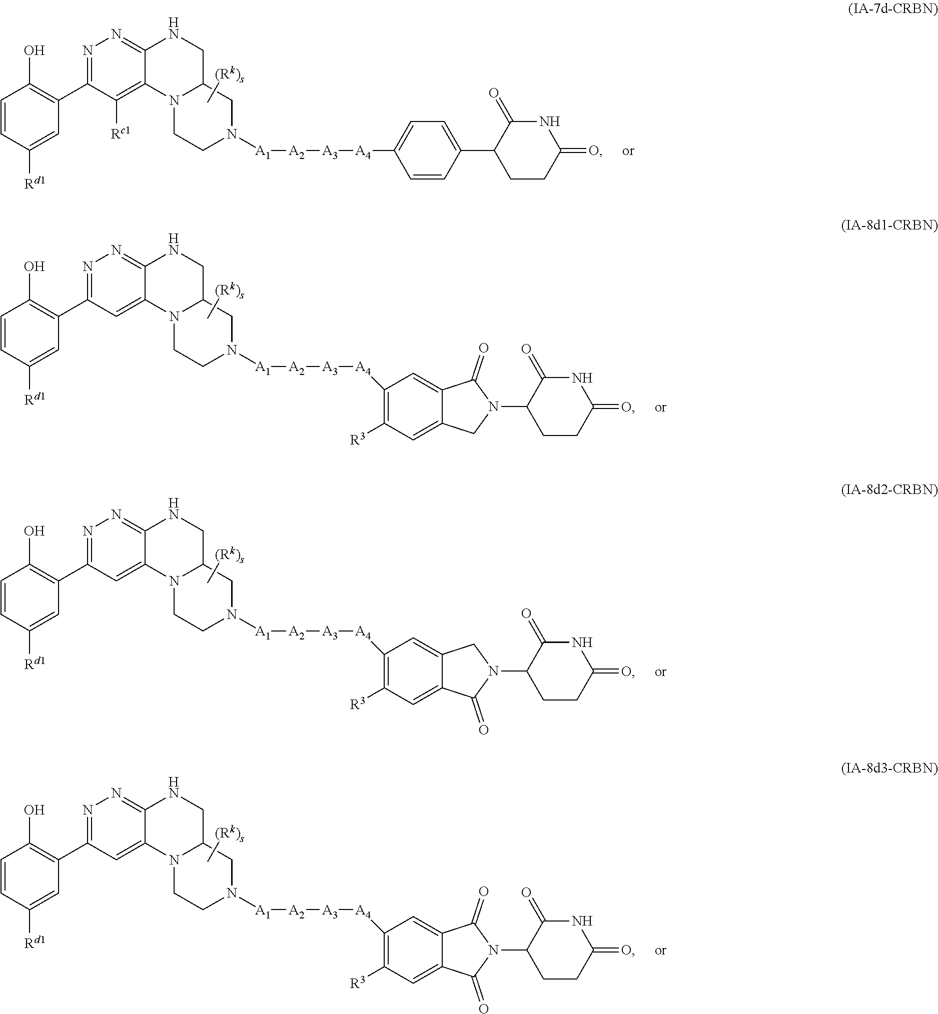

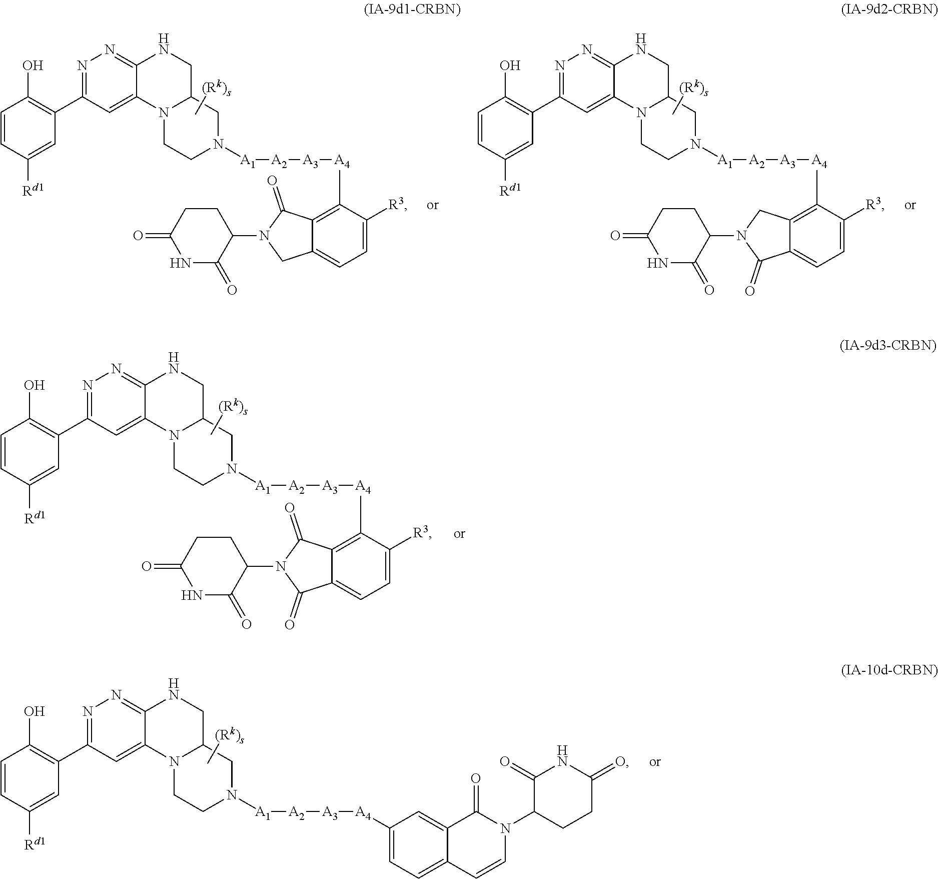

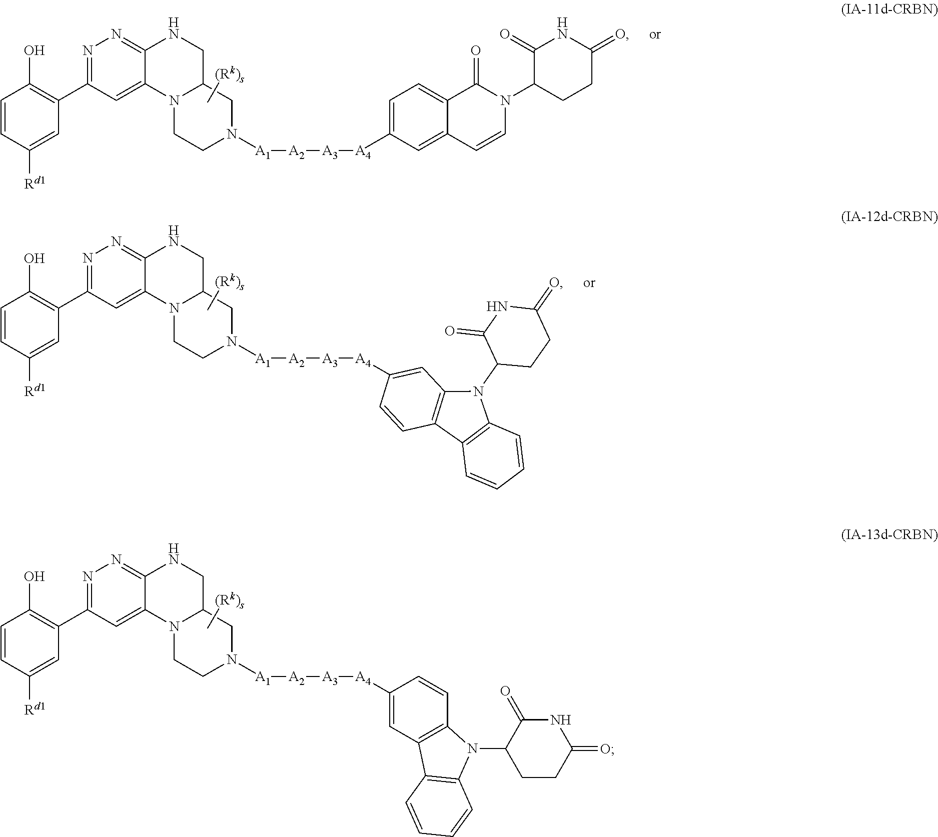

wherein each R^{k} is independently H or C₁₋₆alkyl;
s is 0, 1, 2, 3 or 4;
R^{d1} is H or F;
R₃ is H or F;
A₁ is —CR¹R² or —C═O;
A₂ is 3-8 membered heterocycloalkyl or 3-8 membered cycloalkyl;
A₃ is —CR¹R² or —C═O; and
A₄ is 3-8 membered heterocycloalkyl or 3-8 membered cycloalkyl.

In some embodiments of the degrader compounds of Formula IA-7c-CRBN, Formula IA-8c-CRBN, Formula IA-9c-CRBN, Formula IA-10c-CRbN, Formula IA-11c-CRBN, Formula IA-12c-CRBN or Formula IA-13c-CRBN, R^{d1} is H or F. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, R^{d1}is H. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, R^{d1} is F.

In some embodiments of the degrader compounds of Formula IA-7c-CRBN, Formula IA-8c-CRBN, Formula IA-9c-CRBN, Formula IA-10c-cRBN, Formula IA-11c-CRBN, Formula IA-12c-CRBN or Formula IA-13c-CRBN, R₃ is H or F. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, R₃ is H. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, R₃ is F.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —CR¹R² or —C═O. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —CR¹R². In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —C═O. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₁ is —CH₂.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is 3-8 membered heterocycloalkyl or 3-8 membered cycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a 3-8 membered heterocycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a 3-8 membered cycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is —CR¹R² or —C═O. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₃ is —CR¹R². In some embodiments of the degrader compounds of Formula IA-7c-CRBN, Formula IA-8c-CRBN, Formula IA-9c-CRBN, Formula IA-10c-CRBN, Formula IA-11 c-CRBN, Formula IA-12c-CRBN or Formula IA-13c-CRBN, A₃ is —C═O.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is 3-8 membered heterocycloalkyl or 3-8 membered cycloalkyl. In some embodiments of the degrader compounds of Formula IA-7c-CRBN, Formula IA-8c-CRBN, Formula IA-9c-CRBN, Formula IA-10c-CRBN, Formula IA-11 c-CRBN, Formula IA-12c-CRBN or Formula IA-13c-CRBN, A₄ is a 3-8 membered heterocycloalkyl. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is a 3-8 membered cycloalkyl.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a piperidine, a piperazine, an azetidine or a pyrrolidine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a piperidine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a piperazine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is a pyrrolidine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₂ is an azetidine.

In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is a piperidine, a piperazine, an azetidine or a pyrrolidine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is a piperidine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is a piperazine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is a pyrrolidine. In some embodiments of the degrader compounds of Formula IA-7c-CRBN, Formula IA-8c-CRBN, Formula IA-9c-CRBN, Formula IA-10c-CRBN, Formula IA-11c-CRBN, Formula IA-12c-CRBN or Formula IA-13c-CRBN, A₄ is an azetidine.

In some embodiments, the degrader compounds of Formula I are those having the Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN:

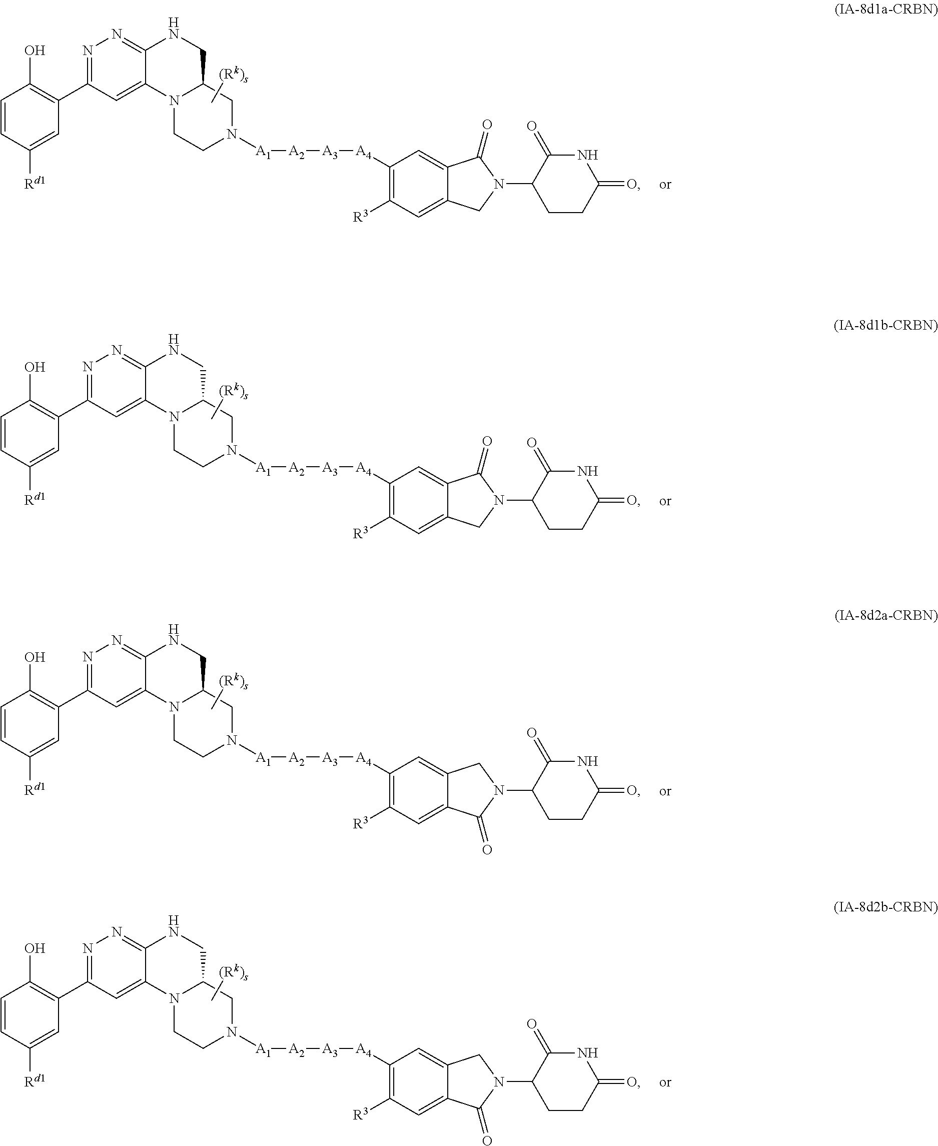

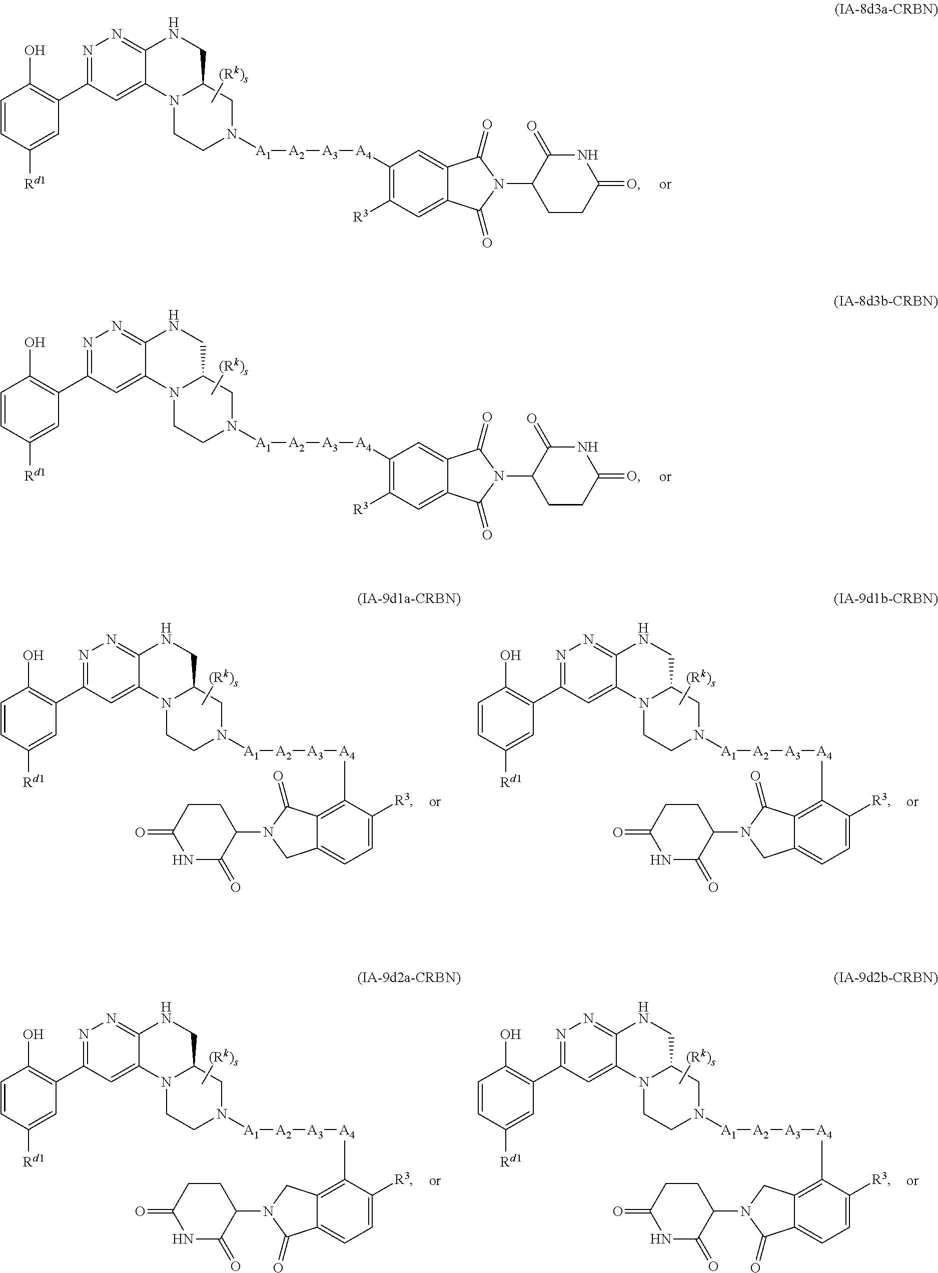

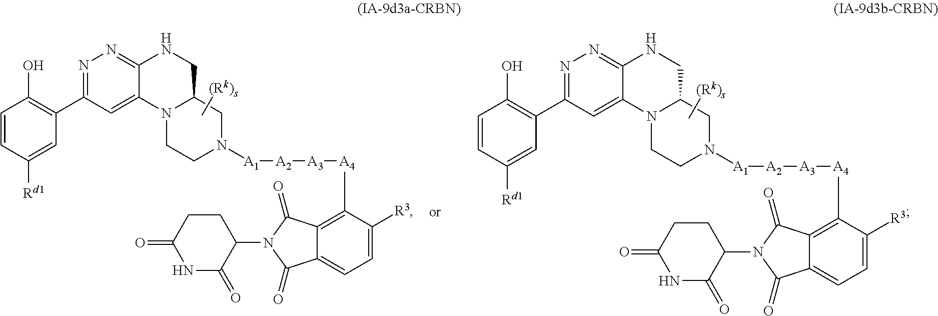

wherein
each R^{k} is independently H or C₁₋₆alkyl;
s is 0, 1, 2, 3 or 4;
R^{d1} is H or F;
R³ is H or F;
A₁ is —CH₂ or —C═O;
A₂ is 3-8 membered heterocycloalkyl or 3-8 membered cycloalkyl;
A₃ is —CR¹R² or —C═O; and
A₄ is 3-8 membered heterocycloalkyl or 3-8 membered cycloalkyl.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, R^{d1} is H or F. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, R^{d1} is H. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, R^{d1} is F.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, R₃ is H or F. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, R₃ is H. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, R₃ is F.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₁ is —CH₂ or —C═O. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₁ is —CH₂. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₁ is —C═O.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is 3-8 membered heterocycloalkyl or 3-8 membered cycloalkyl. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is 3-8 membered heterocycloalkyl. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is 3-8 membered cycloalkyl.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is a piperidine, a piperazine, an azetidine or a pyrrolidine.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is a piperidine. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is a piperazine. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is an azetidine. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₂ is a pyrrolidine.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₃ is —CR¹R² or —C═O. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₃ is —CR¹R². In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₃ is —C═O.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is 3-8 membered heterocycloalkyl or 3-8 membered cycloalkyl. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is 3-8 membered heterocycloalkyl. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is 3-8 membered cycloalkyl.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is a piperidine, a piperazine, an azetidine or a pyrrolidine.

In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is a piperidine. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄is a piperazine. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is an azetidine. In some embodiments of the degrader compounds of Formula IA-8d1a-CRBN, Formula IA-8d1b-CRBN, Formula IA-8d2a-CRBN, Formula IA-8d2b-CRBN, Formula IA-8d3a-CRBN, Formula IA-8d3b-CRBN, Formula IA-9d1a-CRBN, Formula IA-9d1b-CRBN, Formula IA-9d2a-CRBN, Formula IA-9d2b-CRBN, Formula IA-9d3a-CRBN, or Formula IA-9d3b-CRBN, A₄ is a pyrrolidine.

In some embodiments, the degrader compound of Formula (I) has the structure of.

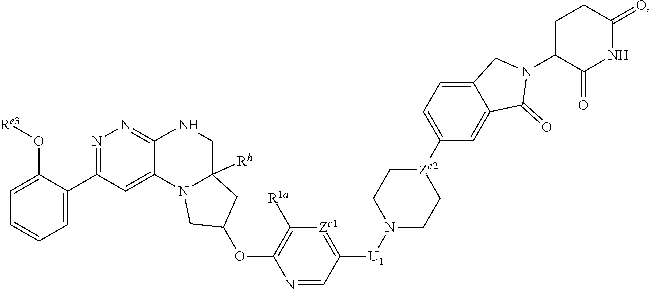

or a pharmaceutically acceptable salt or solvate thereof, wherein
R^{e3} is H, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R^{1a} is H, C₁₋₃ alkyl, or halo;
R^{h} is C₁₋₃ alkyl;
Z^{c1} and Z^{c2} are each independently CH or N; and
U₁ is —CH₂— or —C(O)—.

In some embodiments, R^{1a} in Formula (I) is H, C₁₋₃ alkyl, or halo. In some embodiments, R^{1a} in Formula (I) is H. In some embodiments, R^{1a} in Formula (I) is halo, such as, for example, F, Cl, Br, or I. In other embodiments, R^{1a} in Formula (I) is F. In other embodiments, R^{1a} in Formula (I) is C₁₋₃ alkyl, such as, for example, C₁ alkyl, C₂, alkyl, C₃ alkyl, methyl, ethyl, propyl, isopropyl, and the like. In yet other embodiments, R^{1a} in Formula (I) is methyl. In some embodiments, R^{h} in Formula (I) is C₁₋₃ alkyl, such as, for example, C₁ alkyl, C₂ alkyl, C₃ alkyl, methyl, ethyl, propyl, isopropyl, and the like. In some embodiments, R^{h} in Formula (I) is methyl. In other embodiments, R^{h} in Formula (I) is ethyl. In some embodiments, R^{h} in Formula (I) is propyl. In some embodiments, Z^{c1} in Formula (I) is CH or N. In some embodiments, Z^{c1} in Formula (I) is CH. In other embodiments, Z^{c1} in Formula (I) is N. In some embodiments, Z^{c2} in Formula (I) is CH or N. In some embodiments, Z^{c2}in Formula (I) is CH. In other embodiments, Z^{c2} in Formula (I) is N. In some embodiments, A in Formula (I) is CH₂ or CO (i.e., C═O). In some embodiments, A in Formula (I) is CH₂. In other embodiments, A in Formula (I) is CO. In some embodiments, R^{e3} is H, —C(O)R^{f}, or —P(O)(OR^{g})₂, wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl. In some embodiments, R^{e3} is —CH₂—O—P(O)(OR^{g})₂, wherein each R^{g} is independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl. In some embodiments, each R^{g} is H.

In some embodiments, the degrader compound of Formula (I) has the structure of:

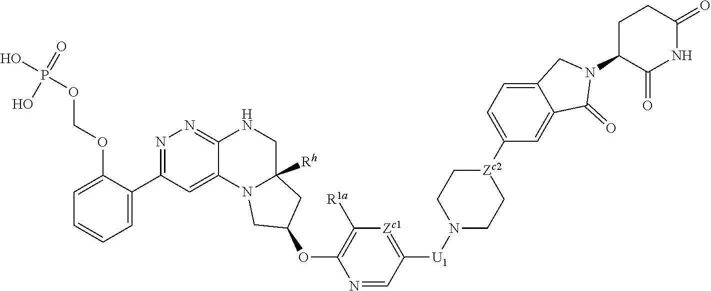

or a pharmaceutically acceptable salts or solvates thereof; wherein
R^{1a} is H, methyl, or F;
R^{h} is methyl or ethyl;
Z^{c1} and Z^{c2} are each independently CH or N; and
U₁ is —CH₂— or —C(O)—.
   In some embodiments, R^{h} is ethyl, R^{1a} methyl, Z^{c1} is CH, U₁ is —CH₂—, and Z^{c2} is N.

In some embodiments, the degrader compound of Formula (I) has the structure of:

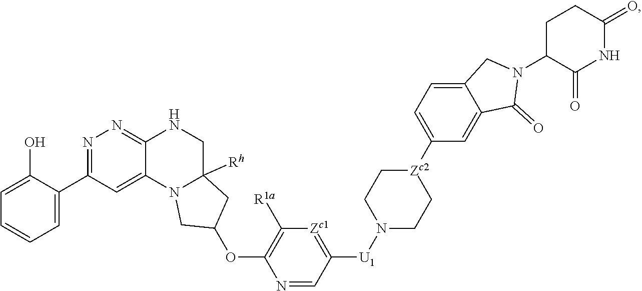

or a pharmaceutically acceptable salt or solvate thereof, wherein
R^{1a} is H, C₁₋₃ alkyl, or halo;
R^{h} is C₁₋₃ alkyl;
Z^{c1} and Z^{c2} are each independently CH or N; and
U₁ is —CH₂— or —C(O)—.

In some embodiments, R^{1a} in Formula (I) is H, C₁₋₃ alkyl, or halo. In some embodiments, R^{1a} in Formula (I) is H. In some embodiments, R^{1a} in Formula (I) is halo, such as, for example, F, Cl, Br, or I. In other embodiments, R^{1a} in Formula (I) is F. In other embodiments, R^{1a} in Formula (I) is C₁₋₃ alkyl, such as, for example, C₁ alkyl, C₂, alkyl, C₃ alkyl, methyl, ethyl, propyl, isopropyl, and the like. In yet other embodiments, R^{1a} in Formula (I) is methyl. In some embodiments, R^{h} in Formula (I) is C₁₋₃ alkyl, such as, for example, C₁ alkyl, C₂ alkyl, C₃ alkyl, methyl, ethyl, propyl, isopropyl, and the like. In some embodiments, R^{h} in Formula (I) is methyl. In other embodiments, R^{h} in Formula (I) is ethyl. In some embodiments, R^{h} in Formula (I) is propyl. In some embodiments, Z^{c1} in Formula (I) is CH or N. In some embodiments, Z^{c1}in Formula (I) is CH. In other embodiments, Z^{c1} in Formula (I) is N. In some embodiments, Z^{c2} in Formula (I) is CH or N. In some embodiments, Z^{c2}in Formula (I) is CH. In other embodiments, Z,2 in Formula (I) is N. In some embodiments, A in Formula (I) is CH₂ or CO (i.e., C═O). In some embodiments, A in Formula (I) is CH₂. In other embodiments, A in Formula (I) is CO.

In some embodiments, the degrader compound of Formula (I) has the structure of:

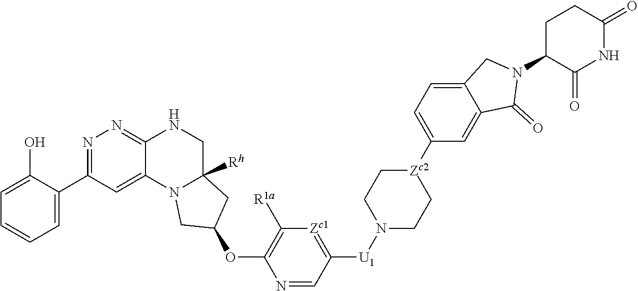

or a pharmaceutically acceptable salts or solvates thereof; wherein
R^{1a} is H, methyl, or F;
R^{h} is methyl or ethyl;
Z^{c1} and Z^{c2} are each independently CH or N; and
U₁ is —CH₂— or —C(O)—.

In some embodiments, R^{1a} in Formula (I) is H, methyl, or F. In some embodiments, R^{1a} in Formula (I) is H. In other embodiments, R^{1a} in Formula (I) is F. In yet other embodiments, R^{1a} in Formula (I) is methyl.

In some embodiments, R^{h} in Formula (I) is methyl or ethyl. In some embodiments, R^{h} in Formula (I) is methyl. In other embodiments, R^{h} in Formula (I) is ethyl. In other embodiments, R^{h} in Formula (I) is propyl.

In some embodiments, Z^{c1} in Formula (I) is CH or N. In some embodiments, Z^{c1}in Formula (I) is CH. In other embodiments, Z^{c1} in Formula (I) is N.

In some embodiments, Z^{c2} in Formula (I) is CH or N. In some embodiments, Z,2 in Formula (I) is CH. In other embodiments, Z,2 in Formula (I) is N.

In some embodiments, A in Formula (I) is CH₂ or CO. In some embodiments, Ain Formula (I) is CH₂. In other embodiments, A in Formula (I) is CO.

In another aspect, the disclosure is directed to a compound of Formula (I) that is:
(S)-3-(6-(4-((6-(((6aS,8R)-2-(2-Hydroxyphenyl)-6a-methyl-5, 6,6a,7,8,9-hexahydropyrrolo[1′,2′:4,5]pyrazino[2,3-c]pyridazin-8-yl)oxy)pyridin-3-yl)methyl)piperazin-1-yl)-1-oxoisoindolin-2-yl)piperidine-2,6-dione;
(S)-3-(6-(4-((5-Fluoro-6-(((6aS,8R)-2-(2-hydroxyphenyl)-6a-methyl-5,6,6a,7,8,9-hexahydropyrrolo[1′,2′:4,5]pyrazino[2,3-c]pyridazin-8-yl)oxy)pyridin-3-yl)methyl)piperazin-1-yl)-1-oxoisoindolin-2-yl)piperidine-2,6-dione;
(S)-3-(6-(1-((6-(((6aS,8R)-2-(2-Hydroxyphenyl)-6a-methyl-5, 6,6a,7,8,9-hexahydropyrrolo[1′,2′:4,5]pyrazino[2,3-c]pyridazin-8-yl)oxy)-5-methylpyridin-3-yl)methyl)piperidin-4-yl)-1-oxoisoindolin-2-yl)piperidine-2,6-dione;
(S)-3-(6-(4-((6-(((6aS,8R)-2-(2-Hydroxyphenyl)-6a-methyl-5, 6,6a,7,8,9-hexahydropyrrolo[1′,2′:4,5]pyrazino[2,3-c]pyridazin-8-yl)oxy)-5-methylpyridin-3-yl)methyl)piperazin-1-yl)-1-oxoisoindolin-2-yl)piperidine-2,6-dione;
(S)-3-(6-(4-(6-(((6aS,8R)-2-(2-Hydroxyphenyl)-6a-methyl-5,6,6a,7,8,9-hexahydropyrrolo[1′,2′:4,5]pyrazino[2,3-c]pyridazin-8-yl)oxy)-5-methylnicotinoyl)piperazin-1-yl)-1-oxoisoindolin-2-yl)piperidine-2,6-dione;
(3S)-3-[5-[4-[[6-[[(4R,6S)-6-Ethyl-12-(2-hydroxyphenyl)-2,8,10,11-tetrazatricyclo-[7.4.0.02,6]trideca-1(9),10,12-trien-4-yl]oxy]-5-methyl-3-pyridinyl]methyl]piperazin-1-yl]-3-oxo-1H-isoindol-2-yl]piperidine-2,6-dione;
(S)-3-(6-(4-(6-(((6aS,8R)-6a-Ethyl-2-(2-hydroxyphenyl)-5,6,6a,7,8,9-hexahydropyrrolo[1′,2′:4,5]pyrazino[2,3-c]pyridazin-8-yl)oxy)-5-methylnicotinoyl)piperazin-1-yl)-1-oxoisoindolin-2-yl)piperidine-2,6-dione;
(S)-3-(6-(4-((5-(((6aS,8R)-6a-Ethyl-2-(2-hydroxyphenyl)-5,6,6a,7,8,9-hexahydropyrrolo[1′,2′:4,5]pyrazino[2,3-c]pyridazin-8-yl)oxy)-6-methylpyrazin-2-yl)methyl)piperazin-1-yl)-1-oxoisoindolin-2-yl)piperidine-2,6-dione;
(S)-3-(6-(4-((6-(((6aS,8R)-6a-Ethyl-2-(2-hydroxyphenyl)-5,6,6a,7,8,9-hexahydropyrrolo[1′,2′:4,5]pyrazino[2,3-c]pyridazin-8-yl)oxy)pyridin-3-yl)methyl)piperazin-1-yl)-1-oxoisoindolin-2-yl)piperidine-2,6-dione;
(S)-3-(6-(1-((6-(((6aS,8R)-6a-ethyl-2-(2-hydroxyphenyl)-5,6,6a,7,8,9-hexahydropyrrolo[1′,2′:4,5]pyrazino[2,3-c]pyridazin-8-yl)oxy)pyridin-3-yl)methyl)piperidin-4-yl)-1-oxoisoindolin-2-yl)piperidine-2,6-dione;
   or a pharmaceutically acceptable salt thereof.

In some embodiments, the degrader compound has the structure of:

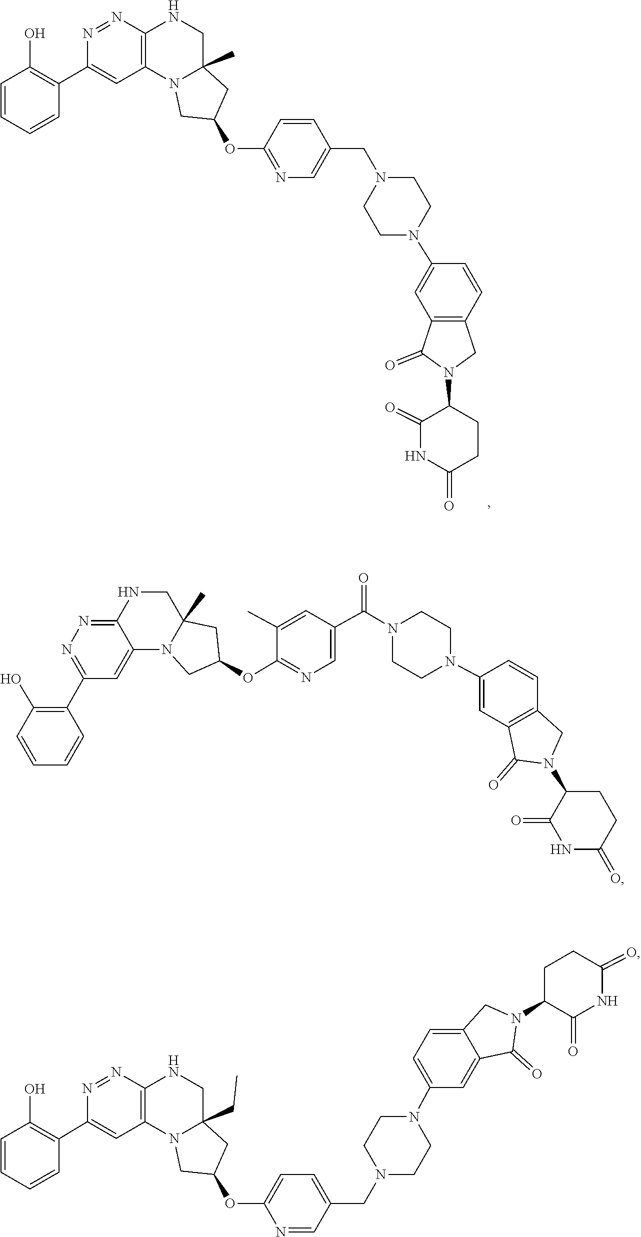 [SMILES: C[C@]12CNc3nnc(-c4ccccc4O)cc3N1C[C@H](Oc1ccc(CN3CCN(c4ccc5c(c4)C(=O)N([C@H]4CCC(=O)NC4=O)C5)CC3)cn1)C2 ; Cc1cc(C(=O)N2CCN(c3ccc4c(c3)C(=O)N([C@H]3CCC(=O)NC3=O)C4)CC2)cnc1O[C@H]1CN2c3cc(-c4ccccc4O)nnc3NC[C@]2(C)C1 ; CC[C@]12CNc3nnc(-c4ccccc4O)cc3N1C[C@H](Oc1ccc(CN3CCN(c4ccc5c(c4)C(=O)N([C@H]4CCC(=O)NC4=O)C5)CC3)cn1)C2]

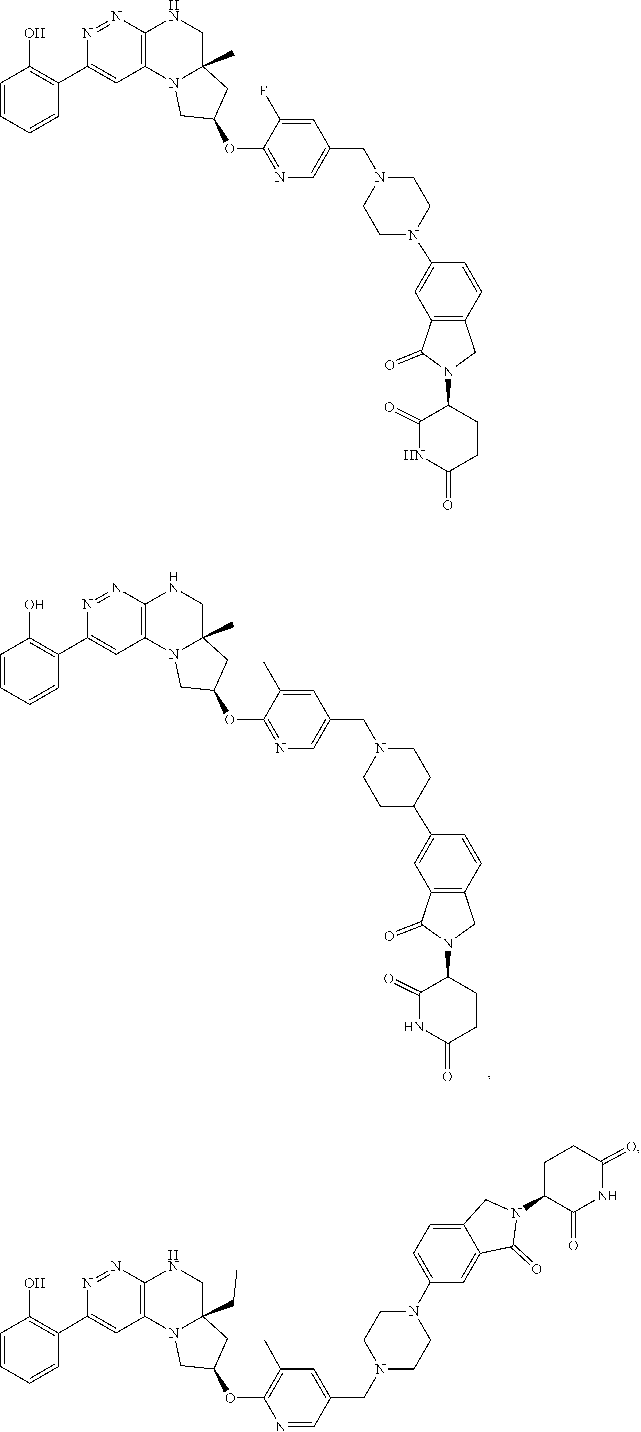 [SMILES: C[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)cc1F)C2 ; Cc1cc(CN2CCC(c3ccc4c(c3)C(=O)N([C@H]3CCC(=O)NC3=O)C4)CC2)cnc1O[C@H]1CN2c3cc(-c4ccccc4O)nnc3NC[C@]2(C)C1 ; CC[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)cc1C)C2]

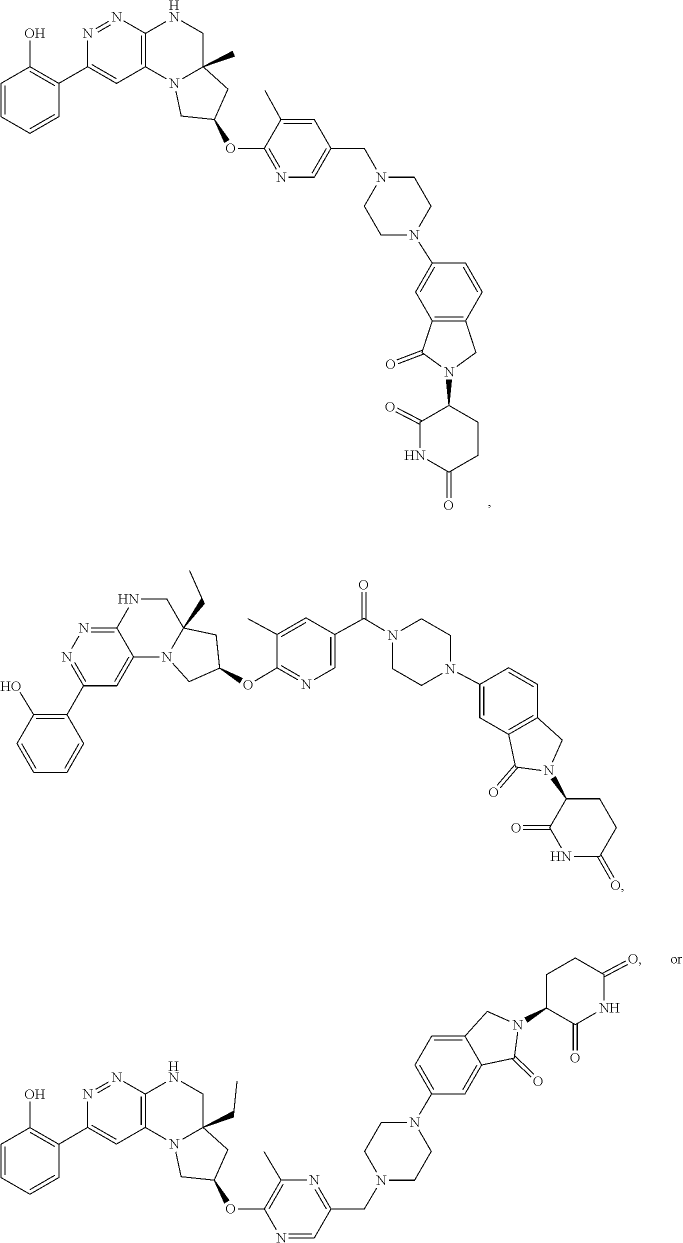 [SMILES: Cc1cc(CN2CCN(c3ccc4c(c3)C(=O)N([C@H]3CCC(=O)NC3=O)C4)CC2)cnc1O[C@H]1CN2c3cc(-c4ccccc4O)nnc3NC[C@]2(C)C1 ; CC[C@]12CNc3nnc(-c4ccccc4O)cc3N1C[C@H](Oc1ncc(C(=O)N3CCN(c4ccc5c(c4)C(=O)N([C@H]4CCC(=O)NC4=O)C5)CC3)cc1C)C2 ; CC[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)nc1C)C2]

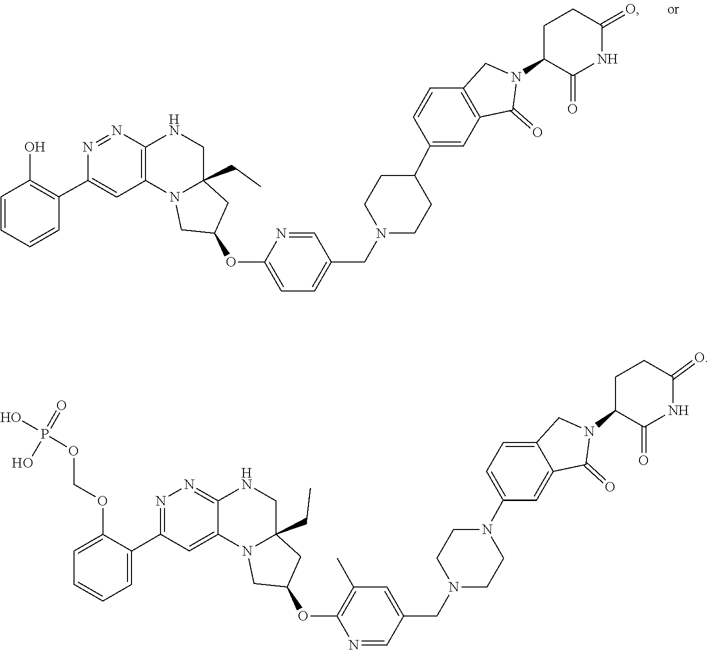 [SMILES: CC[C@]12CNc3nnc(-c4ccccc4O)cc3N1C[C@H](Oc1ccc(CN3CCC(c4ccc5c(c4)C(=O)N([C@H]4CCC(=O)NC4=O)C5)CC3)cn1)C2 ; CC[C@]12CNc3nnc(-c4ccccc4OCOP(=O)(O)O)cc3N1C[C@H](Oc1ncc(CN3CCN(c4ccc5c(c4)C(=O)N([C@H]4CCC(=O)NC4=O)C5)CC3)cc1C)C2]

In some embodiments, the degrader compound has the structure of:

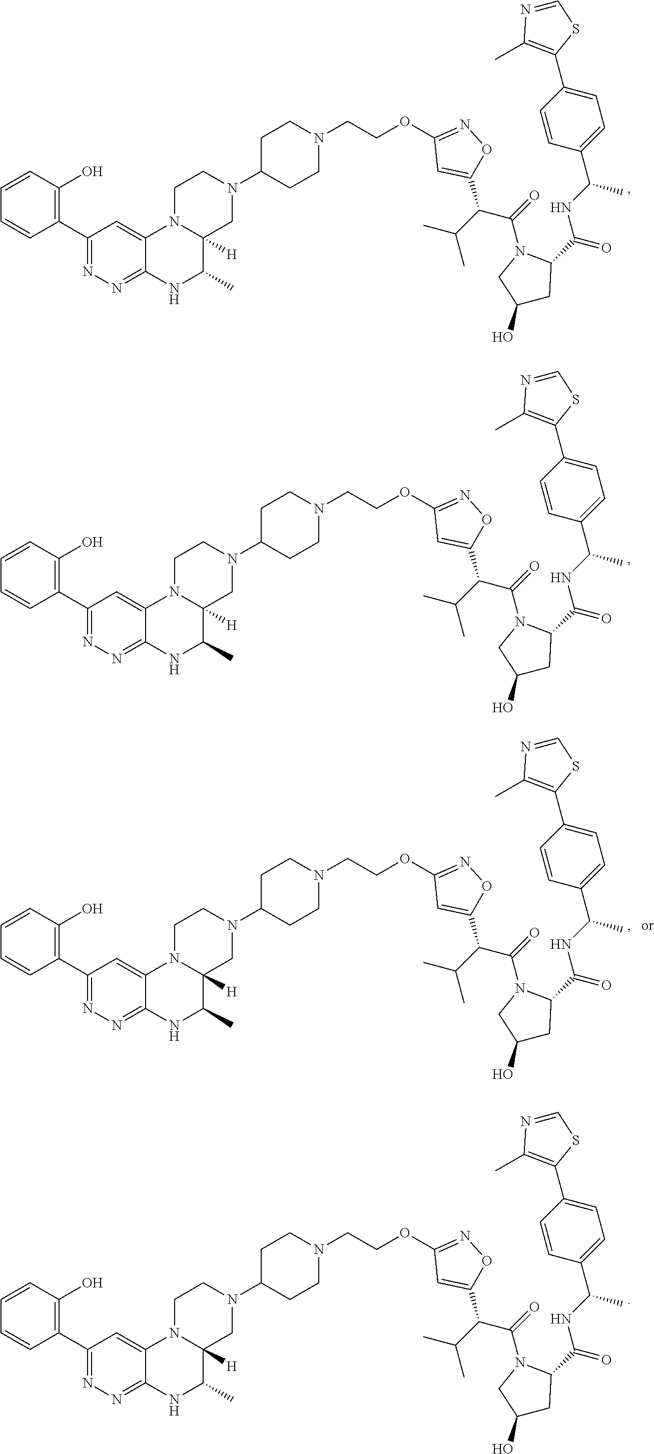

In some embodiments, the degrader compound has the structure of:

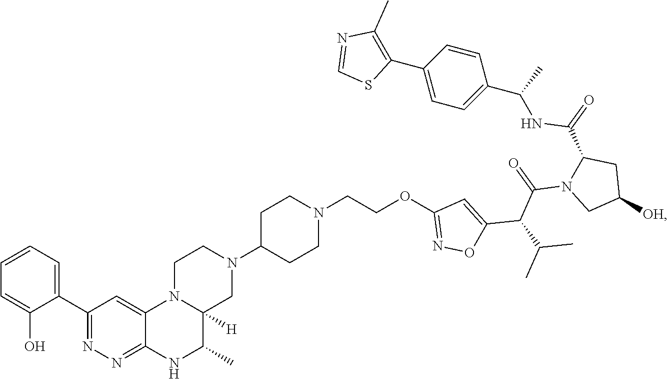 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@@H](C)[C@@H]5C4)CC3)no2)C(C)C)cc1]

or a pharmaceutically acceptable salt thereof.

In some embodiments, the degrader compound of Formula (I) has the structure of:

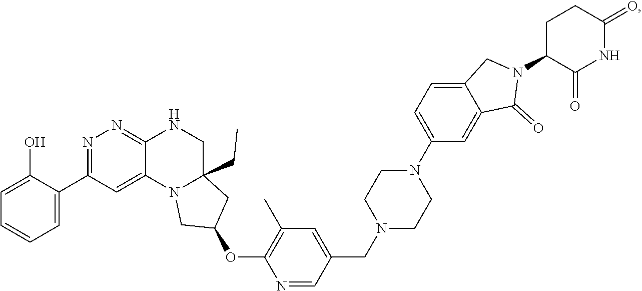 [SMILES: CC[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)cc1C)C2]

or a pharmaceutically acceptable salt or solvate thereof.

In some embodiments, the degrader compound of Formula (I) has the structure of:

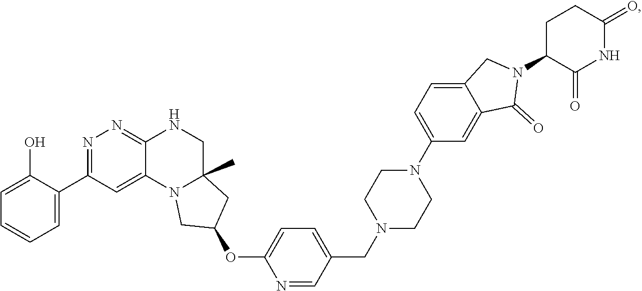 [SMILES: C[C@]12CNc3nnc(-c4ccccc4O)cc3N1C[C@H](Oc1ccc(CN3CCN(c4ccc5c(c4)C(=O)N([C@H]4CCC(=O)NC4=O)C5)CC3)cn1)C2]

or a pharmaceutically acceptable salt or solvate thereof.

### Prodrugs

In some embodiments, the degrader compound as described herein is in the form of a prodrug (e.g., a pharmacologically acceptable derivative of the degrader compounds described herein). In some embodiments, the prodrug is in the form of a phosphate. In some embodiments, the prodrug is in the form of a carbohydrate. In some embodiments, the carbohydrate is selected from glucose, galactose, lactose, mannose, fucose, n-acetylglucosamine, xylose, sialic acid, or glucuronic acid. In some embodiments, the carbohydrate is glucuronic acid. In some embodiments, at least one functional group of the degrader compound is used as the site of attachment to form the prodrug. In some embodiments, the functional group is a hydroxyl group, a hydroxy group of a phenol, an amine group, a carboxylic acid group, a phosphate/phosphonate group, and a carbonyl group.

In some embodiments, the degrader compound in the form of a prodrug comprises a small molecule E3 Ubiquitin Ligase binding moiety that binds a Cereblon E3 Ubiquitin Ligase.

In some embodiments, the degrader compound in the form of a prodrug comprises a small molecule E3 Ubiquitin Ligase binding moiety that binds a Von Hippel-Lindau E3 Ubiquitin Ligase. In some embodiments, the degrader compound in the form of a phosphate has the structure of:

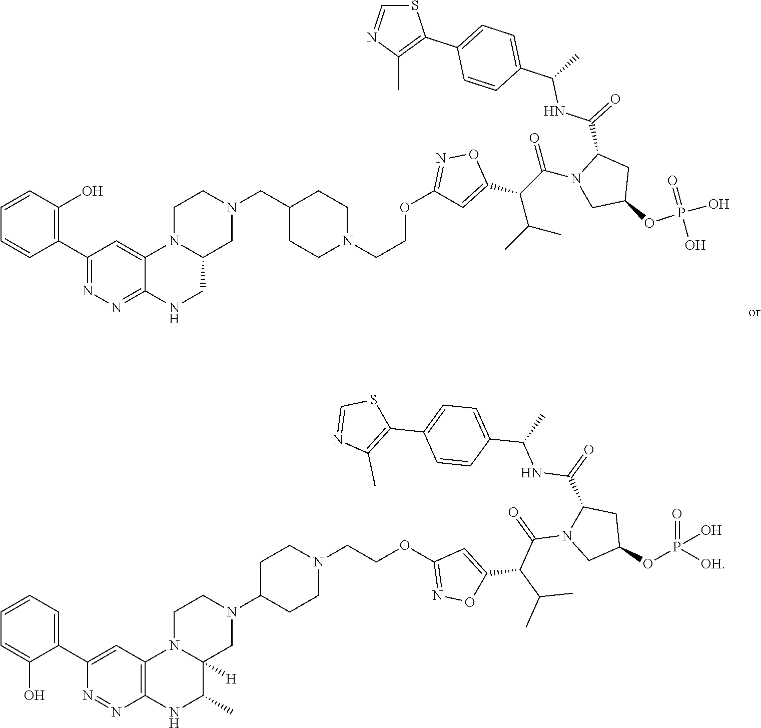 [SMILES: Cc1ncsc1-c1ccc([C@H](C)NC(=O)[C@@H]2C[C@@H](OP(=O)(O)O)CN2C(=O)[C@@H](c2cc(OCCN3CCC(CN4CCN5c6cc(-c7ccccc7O)nnc6NC[C@H]5C4)CC3)no2)C(C)C)cc1 ; Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7O)nnc6N[C@@H](C)[C@@H]5C4)CC3)no2)C(C)C)cc1]

It will be apparent that the degrader compounds and resulting conjugates and degrader-linker compounds of the invention, including all subgenera described herein, may have multiple stereogenic centers. As a result, there exist multiple stereoisomers (enantiomers and diastereomers) of the compounds (and subgenera described herein) and conjugates. The present disclosure contemplates and encompasses each stereoisomer of any compound or conjugate encompassed by the disclosure as well as mixtures of said stereoisomers.

References to formula I or subgenera thereof (e.g., formula IA, IA-1, IA-2, IA-3, IA-4, IA-5, IA-6, IA-7-VHL, IA-8-VHL, IA-9-VHL, IA-10-VHL, IA-7-CRBN, IA-8-CRBN, IA-9-CRBN, etc.) are meant to encompass the identified formula and all applicable subgenera.

Pharmaceutically acceptable salts and solvates of the compounds and conjugates of the disclosure (including all subgenera described herein) are also within the scope of the disclosure.

Also contemplated herein are isotopic variants and isotopically labeled forms of the compounds, such as an Antibody Drug Conjugate compound or a Degrader-Linker compound (including all subgenera described herein). Isotopic variants and isotopically labeled forms of the compounds described herein have structures as depicted and/or described herein, except that one or more atoms are replaced by an atom having a selected atomic mass or mass number.

Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C, are contemplated. Such compounds are synthesized by means well known in the art, for example by employing starting materials in which one or more hydrogens have been replaced by deuterium.

### Antibody Drug Conjugates

In some embodiments, the Antibody Drug Conjugate compound is represented by the structure of:

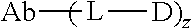

wherein,
Ab is an antibody or an antigen-binding fragment thereof,
L is a linker;
D is a degrader compound of Formula (I):

PTM-ULM (I),
wherein,
   PTM is a moiety of Formula IA:

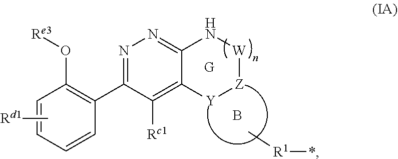

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase; and subscript z is an integer ranging from 1 to 14.

In some embodiments, R¹ is a covalent bond that links PTM and ULM. In some embodiments, R¹ is a chemical moiety that links PTM and ULM. In some embodiments, n is 0. In some embodiments, n is 1. In some embodiments, n is 0 or 1. In some embodiments, n is 2. In some embodiments, n is 3. In some embodiments, each W is independently optionally substituted —CH₂. In some embodiments, each W is independently —C(O)—. In some embodiments, each W is independently —S(O)—. In some embodiments, each W is independently —S(O)₂—. In some embodiments, R^{c1} and R^{d1} are both hydrogen. In some embodiments, R^{e3} is hydrogen. In some embodiments, Z and Y are both N. In some embodiments, Y and Z are each independently CR^{h}, wherein R^{h}=H, C₁₋₃ alkyl, or absent. In some embodiments, one of Z and Y is N, and the other is CR^{h} wherein R^{h}=H, C₁₋₃ alkyl, or absent. In some embodiments, R¹ is attached to Z, and Z is C and Y is N or CR^{h} wherein R^{h} is H or C₁₋₃ alkyl. In some embodiments, R¹ is attached to Y, and Y is C and Z is N or CR^{h} wherein R^{h} is H or C₁₋₃ alkyl. In some embodiments, B is an optionally substituted 5-7 membered cycloalkyl ring. In some embodiments, B is, an optionally substituted 5-7 membered heteroaryl ring. In some embodiments, B is an optionally substituted 5-7 membered heterocyclic ring. In some embodiments, ULM binds to a Von Hippel-Lindau E3 Ubiquitin Ligase. In some embodiments, ULM binds to a Cereblon E3 Ubiquitin Ligase. In some embodiments, z is 1. In some embodiments, z is 2. In some embodiments, z is 4. In some embodiments, z is 8. In some embodiments, z is an integer ranging from 1 to 8. In some embodiments, z is 10. In some embodiments, z is 12. In some embodiments, z is 14.

In some embodiments, the Antibody Drug Conjugate compound is represented by the structure of:

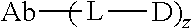

wherein,
Ab is an antibody or an antigen-binding fragment thereof,
D is a degrader compound of Formula (I):

PTM-ULM (I)
wherein,
   PTM is a moiety of Formula IA:

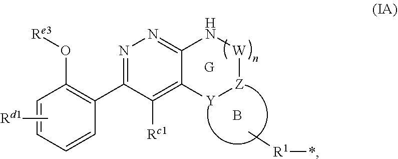

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃. 8 substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (II):

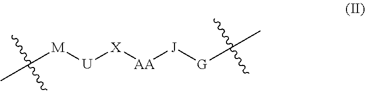

wherein,
   M is selected from the group consisting of:

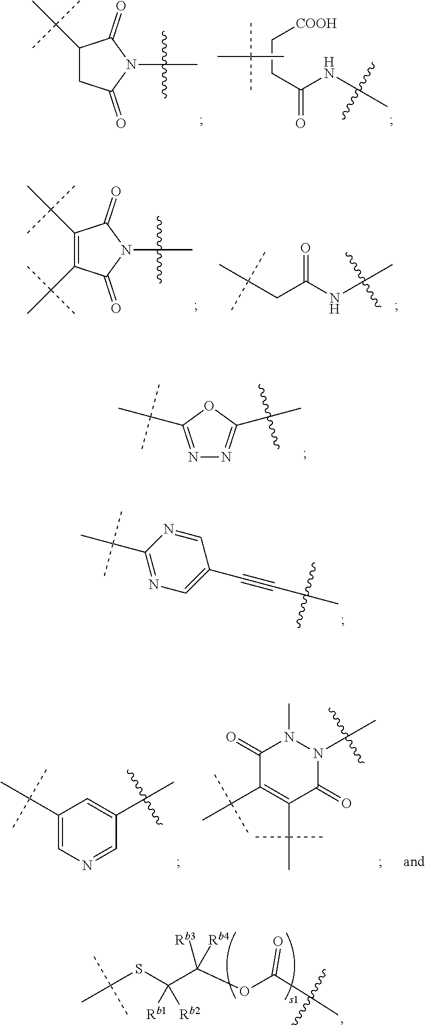

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl, subscript s1 is 0 or 1,
wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—, wherein R^{x5} is hydrogen or C₁-C₆ alkyl and R^{x6} is hydrogen, C₁-C₆ alkyl, or

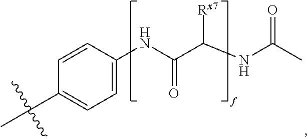

wherein subscript f is an integer ranging from 0 to 4 and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
AA is absent or has the structure of:

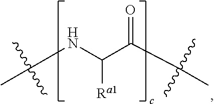

wherein subscript c is an integer ranging from 1 to 12; each R^{a1} is independently —COOH, —NH₂, or an independently selected side chain of an amino acid;
J is absent, —(R^{j3})N(C(R^{j1})(R^{j2}))ₘ₁—, or has the structure of:

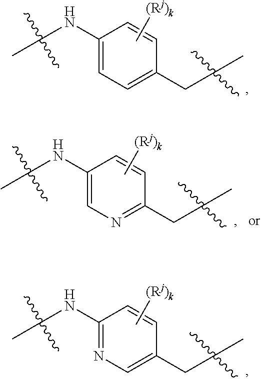

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
G is absent,

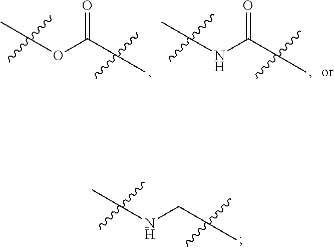

and
wherein the wavy line to M of Formula (II) indicates the point of covalent attachment to Ab and the wavy line to G of Formula (II) indicates the point of covalent attachment to D,
wherein subscript z is an integer ranging from 1 to 14.

In some embodiments, G is

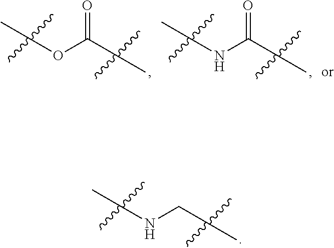

In some embodiments, the wavy line to M of Formula (II) indicates the point of covalent attachment to Ab and the wavy line to G of Formula (II) indicates the point of covalent attachment to D. In some embodiments, the wavy line to G of Formula (II) indicates the point of covalent attachment to a secondary alcohol group or a secondary amine group of D. In some embodiments, the secondary alcohol group is the alcohol group of a phenol-group of D. In so-e embodiments, the secondary amine group is the imido nitrogen of a glutarimide group of D. In some embodiments, the secondary amine group is the amine nitrogen of a piperazine group of D.

In some embodiments, the Antibody Drug Conjugate compound has the structure of:

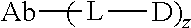

wherein,
Ab is an antibody or an antigen-binding fragment thereof,
D is a degrader compound of Formula (I):

PTM-ULM (I),
wherein,
   PTM is a moiety of Formula IA:

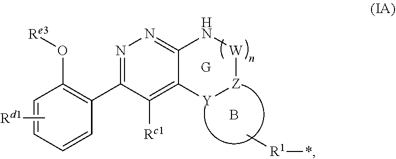

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (IIa):

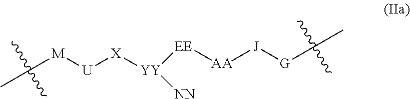

wherein,
   M is selected from the group consisting of:

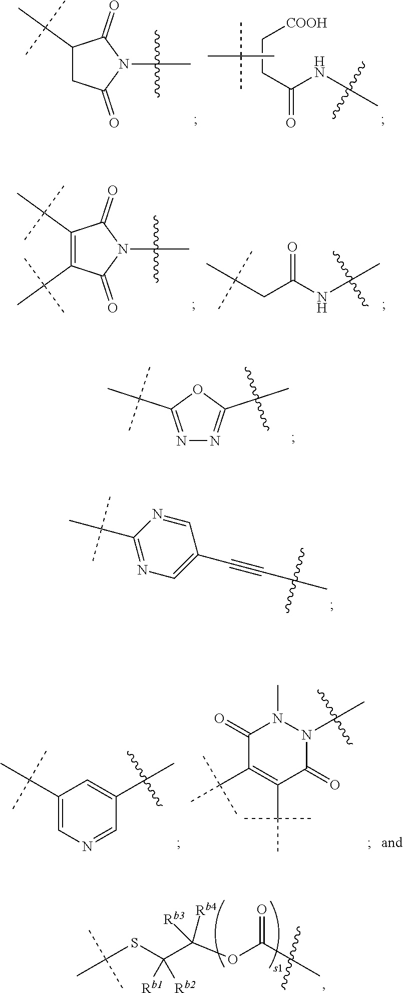

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1,
wherein the dashed line indicates the point of covalent attachment to the Ab and the wavy line indicates the point of covalent attachment to the remainder of the structure of L′;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—, wherein R^{x5} is hydrogen or C₁-C₆ alkyl and R^{x6} is hydrogen, C₁-C₆ alkyl, or

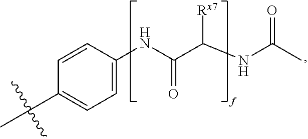

wherein subscript f is an integer ranging from 0 to 4 and each R^{x7} is independently hydrogen, —COOH, —NH₂, C₁-C₆ alkyl, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of:

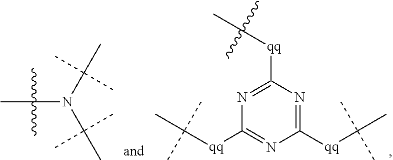

wherein
   each qq is independently —N(R^{y1})— or —O—, wherein R^{y1} is hydrogen or C₁-C₆ alkyl,
   one dashed line indicates the point of covalent attachment to EE when EE is present, or to AA when EE is absent, or to J when AA and EE are absent, or to G when AA, EE, and J are absent, and the other dashed line indicates the point of covalent attachment to NN, wherein the wavy line indicates the point of covalent attachment to X, when X is present, or to U, when X is absent, or to M, when X and U are absent;
NN is —(CH₂CH₂O)_{k′}CH₂CH₂C(O)—Rⁿⁿ¹-, wherein subscript k′ is an integer ranging from 0 to 16 and Rⁿⁿ¹ is hydrogen, C₁-C₆ alkyl, or —OH;
EE is absent or —(CH₂CH₂O)_{x′}CH₂CH₂C(O)—, wherein subscript x′ is an integer ranging from 0 to 16;
AA is absent or has the structure of:

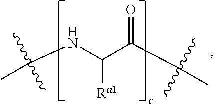

wherein subscript c is an integer ranging from 1 to 12; each R^{a1} is independently —COOH, —NH₂, or an independently selected side chain of an amino acid;
J is independently absent, —(R^{j3})N(C(R^{j1})(R^{j2}))ₘ₁—, or has the structure of:

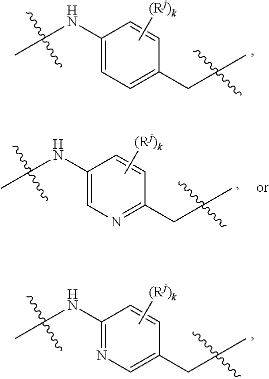

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
G is absent,

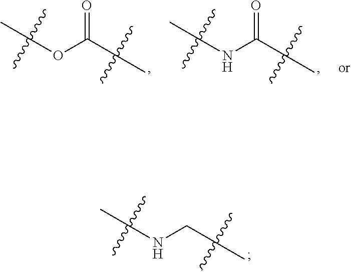

and
wherein the wavy line to M of Formula (IIa) indicates the point of covalent attachment to Ab and the wavy line to G of Formula (IIa) indicates the point of covalent attachment to D. In some embodiments, R¹ is a covalent bond. In some embodiments, R¹ is a chemical moiety represented by the formula -(A)_{q}-, wherein q is an integer from 1 to 14; each A is independently selected from the group consisting of CR^{1a}R^{1b} o, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b}groups, 3-11 membered heterocyclyl optionally substituted with 0-6 R^{1a} and/or R^{1b} groups, aryl optionally substituted with 0-6 R^{1a} and/or R^{1b} groups, and heteroaryl optionally substituted with 0-6 R^{1a} and/or R^{1b} groups, wherein R^{1a}, R^{1b}, R^{1c}, R^{1d} and R^{1e} are each independently, —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC1-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups. In some embodiments, q is 1. In some embodiments, q is 2. In some embodiments, q is 3. In some embodiments, q is 4. In some embodiments, q is an integer from 1 to 4. In some embodiments, q is 5. In some embodiments, q is 6. In some embodiments, q is 7. In some embodiments, q is 8. In some embodiments, q is an integer from 1 to 8. In some embodiments, q is 10. In some embodiments, q is 12. In some embodiments, q is 14.

In some embodiments, ULM binds Von Hippel-Lindau E3 Ubiquitin Ligase. In some embodiments, ULM is a moiety having the Formula ULM-I-VHL:

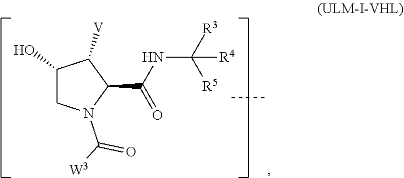

wherein
the dashed line ( [symbol] ) indicates the position of attachment of ULM-I-VHL to R¹;
V is H or F;
R³ is optionally substituted phenyl, optionally substituted napthyl, or an optionally substituted 5-10 membered heteroaryl;
one of R⁴ or R⁵ is H, deuterium, haloalkyl, optionally substituted alkyl, optionally substituted cycloalkyl, optionally substituted heterocycloalkyl, —COR^{dv}, or CON^{e1}R^{e2};
the other of R⁴ or R⁵ is H or deuterium;
or R⁴ and R⁵, together with the carbon atom to which they are both attached, form an optionally substituted 3-5 membered cycloalkyl or heterocyclyl;
W³ is an optionally substituted aryl, optionally substituted heteroaryl, or

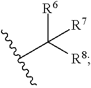

R⁶ and R⁷ are independently H, deuterium, optionally substituted alkyl, optionally substituted cycloalkyl, or optionally substituted haloalkyl,
or R⁶, R⁷, and the carbon atom to which they are attached form an optionally substituted cycloalkyl or optionally substituted heterocyclyl;
R⁸ is an optionally substituted heterocyclyl, optionally substituted heteroaryl, optionally substituted aryl, CONR^{av}R^{bv}, NR^{av}R^{bv},

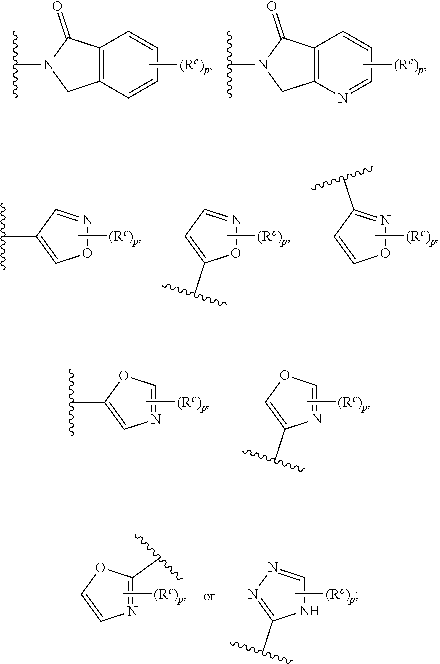

R^{av} is H or optionally substituted alkyl;
R^{bv} is H, —C(O)—* wherein * is a point of attachment to R¹, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{c} is independently H, halo, optionally substituted alkoxy, cyano, optionally substituted alkyl, haloalkyl, or haloalkoxy;
each R^{dv} is independently H, optionally substituted alkyl or NR^{e1}R^{e2};
each R^{e1} and R^{e2} is independently H, deuterium, or optionally substituted alkyl,
or R^{e1} and R^{e2} together with the nitrogen atom to which they are attached form a 4-7 membered heterocyclyl; and
p is 0, 1, 2, 3, or 4.

In some embodiments, V is H. In some embodiments, V is F. In some embodiments, R³ is optionally substituted phenyl. In some embodiments, R³ is optionally substituted napthyl. In some embodiments, R³ is an optionally substituted 5-10 membered heteroaryl. In some embodiments, one of R⁴ or R⁵ is H, deuterium, haloalkyl, optionally substituted alkyl, optionally substituted cycloalkyl, optionally substituted heterocycloalkyl, —COR^{dv}, or CONR^{e1}R^{e2}; and the other of R⁴ or R⁵ is H. In some embodiments, R⁴ and R⁵, together with the carbon atom to which they are both attached, form an optionally substituted 3-5 membered cycloalkyl or heterocyclyl. In some embodiments, W³ is an optionally substituted aryl. In some embodiments, W³ is an optionally substituted heteroaryl. In some embodiments, W³ is

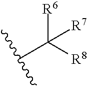

wherein R⁶ and R⁷ are independently H, deuterium, optionally substituted alkyl, optionally substituted cycloalkyl, or optionally substituted haloalkyl. In some embodiments, W³ is

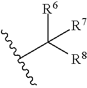

R⁸ wherein R⁶, R⁷, and the carbon atom to which they are attached form an optionally substituted cycloalkyl or optionally substituted heterocyclyl. In some embodiments, R⁸ is an optionally substituted heterocyclyl, optionally substituted heteroaryl, optionally substituted aryl, CONR^{av}R^{bv}, or NR^{av}R^{bv}. In some embodiments, R⁸ is

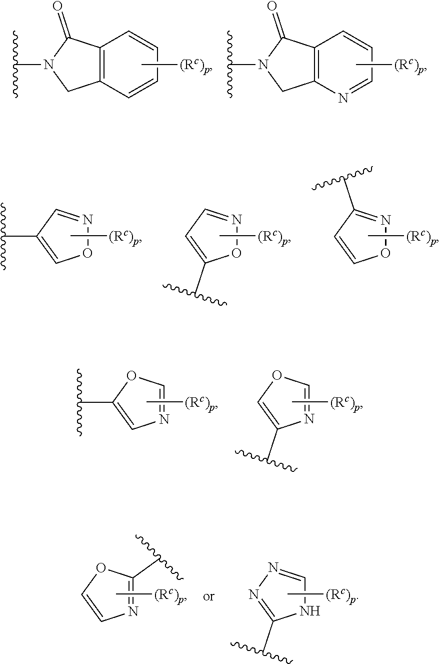

In some embodiments, ULM-I-VHL is a compound of formula:

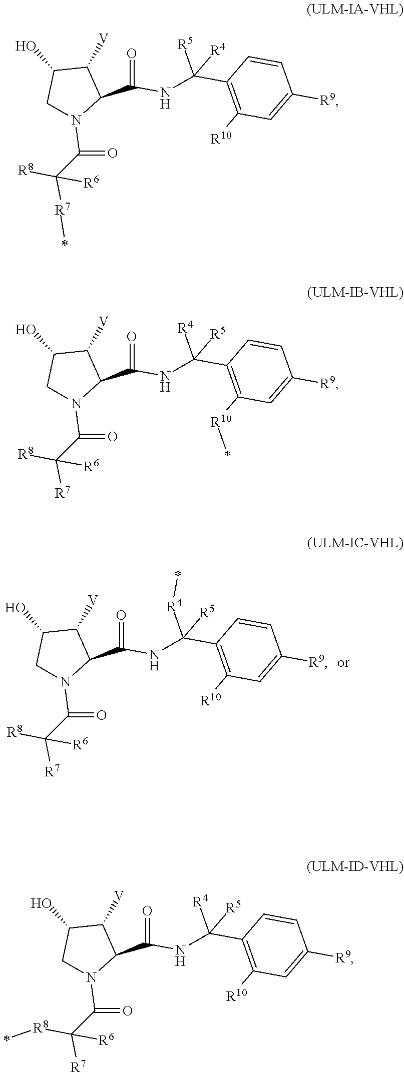

wherein * is a point of attachment of the ULM to R¹.

In some embodiments, ULM-I-VHL is a compound of formula (ULM-IA-VHL). In some embodiments, ULM-I-VHL is a compound of formula (ULM-IB-VHL). In some embodiments, ULM-I-VHL is a compound of formula (ULM-IC-VHL). In some embodiments, ULM-I-VHL is a compound of formula (ULM-ID-VHL).

In some embodiments, D has a structure of:

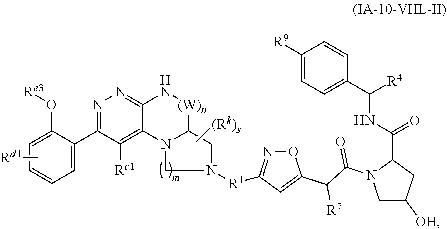

wherein
W is optionally substituted —CH₂—, —C(O)—, —S(O)—, or —S(O)₂—; wherein when n=2 or 3, only one W may be —C(O)—, —S(O)—, or —S(O)₂—;
n=0-3;
m=1-3;
R^{k}=H, deuterium, F, C₁₋₃ alkyl, C₁₋₃ haloalkyl, or C₁₋₄ alkoxyl;
s=0-3;
R^{c1} and R^{d1} are independently H, deuterium, Halo, C₁₋₃ alkyl, C₁₋₃ haloalkyl, or C₁₋₄ alkoxyl;
R^{e3} is H, —C(O)R, —CH₂—O—P(O)(OR^{g})₂, or —P(O)(OR^{g})₂; wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b}groups, —(CR^{1a}R^{1b})₁₋₅, —(CR^{1a}═CR^{1b})—(CR^{1a}R^{1b})₁—.-A- wherein A is O, S, or NR^{1c}—(CR^{1a}R^{1b})₁₋₅-A-(CR^{1a}R^{1b})₁₋₅— wherein A is O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})i5-(CR^{1a}═CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b} groups)-, -(3-11 membered cycloalkyl optionally substituted with 0-6 R^{1a} and/or R^{1b} groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}—(CR^{1a}R^{1b})₁₋₅-(3-11 membered heterocyclyl optionally substituted with 0-6 R^{1a} and/or R^{1b}groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-(3-11 membered heterocyclyl optionally substituted with 0-6 R^{1a} and/or R^{1b} groups)-A- wherein A is O, S, or NR^{1c}—(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b}groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR¹¹, (CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}, —(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b} groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with-0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}-(3-11 membered cycloalkyl optionally substituted with 0-6 R^{1a} and/or R^{1b} groups)-(CR^{1a}R^{1b})₁₋₅₋, or -(3-11 membered heterocyclyl optionally substituted with 0-6 R^{1a} and/or R^{1b} groups)-(CR^{1a}R^{1b})₁₋₅.; or R^{j} is -(3-11 membered heterocyclyl optionally substituted with 0-6 R^{1a} and/or R^{1b} groups)-(CO)—(CR^{1a}R^{1b})₁₋₅-A- wherein A is O, S, or NR^{1c}; -(3-11 membered heterocyclyl optionally substituted with 0-6 R^{1a} and/or R^{1b} groups)-(CO)-A-(CR^{1a}R^{1b})₁₋₅— wherein A is O, S, or NR^{1c}; -A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}; -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-A-(CR^{1a}R^{1b})₁₋₅-A- wherein each A is independently O, S, or NR^{1c}; -(heteroaryl optionally substituted with 0-4 R^{1a} and/or R^{1b} groups)-A-(CR^{1a}R^{1b})₁₋₅— wherein A is O, S, or NR^{1c}; -(3-11 membered heterocyclyl optionally substituted with 0-6 R^{1a} and/or R^{1b}groups)-(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)—(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or —(CO)-(3-11 membered cycloalkyl optionally substituted with 0-6 R^{1a} and/or R^{1b} groups)-(CR^{1a}R^{1b})₁₋₅-A- wherein A is O, S, or NR^{1c};
R⁴ is H, optionally substituted alkyl, optionally substituted C₁-C₆alkyl, or —CH₃;
R⁷ is optionally substituted alkyl, preferably optionally substituted C₁-C₆alkyl, and more preferably C₁-C₆alkyl; and
R⁹ is H, deuterium, halo, —CN, —OH, —NO₂, —NR^{e1}R^{e2}, —OR^{e1}, —CONR^{e1}R^{e2}, —NR^{e1}COR^{e2}, —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R^{1a}, R^{1b}, R^{1c}, and R^{1c} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC1-C₈alkyl)(C₁-C₈alkyl), —P(O)(OC1-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC1-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups; and
each R^{e1} and R^{e2} is independently H, deuterium, or optionally substituted alkyl, or R^{c1} and R^{e2} together with the nitrogen atom to which they are attached form a 4-7 membered heterocyclyl.

In some embodiments, D has a structure of:

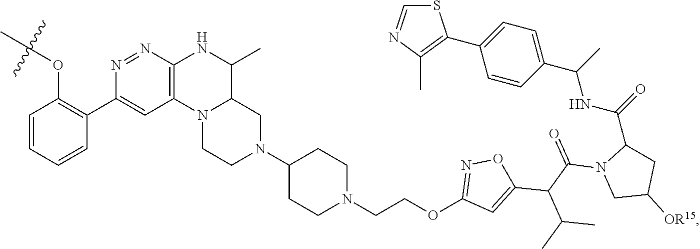

wherein R¹⁵ is hydrogen or —PO₃H₂.

In some embodiments, D has a structure of:

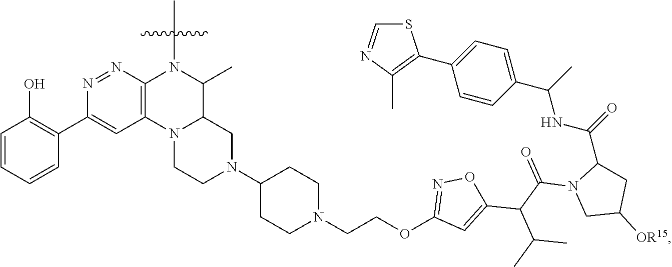

wherein R¹⁵ is hydrogen or —PO₃H₂and the wavy line indicates the point of covalent attachment to L.

In some embodiments, D has a structure of:

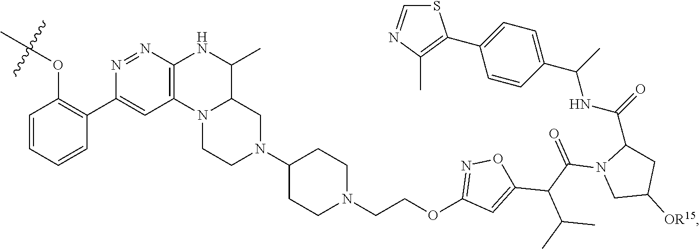

wherein R¹⁵ is hydrogen or —PO₃H₂and the wavy line indicates the point of covalent attachment to L.

In some embodiments, D has a structure of:

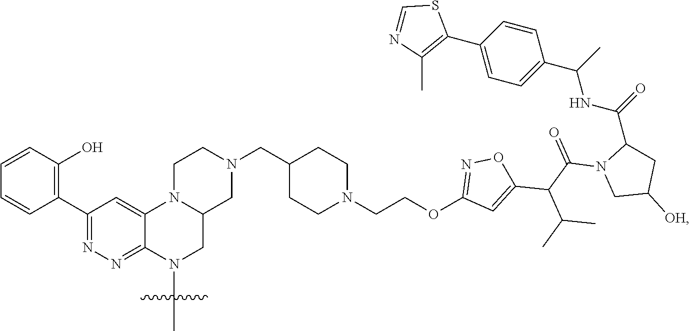 [SMILES: *N1CC2CN(CC3CCN(CCOc4cc(C(C(=O)N5CC(O)CC5C(=O)NC(C)c5ccc(-c6scnc6C)cc5)C(C)C)on4)CC3)CCN2c2cc(-c3ccccc3O)nnc21]

wherein the wavy line indicates the point of covalent attachment to L.

In some embodiments, D has a structure of:

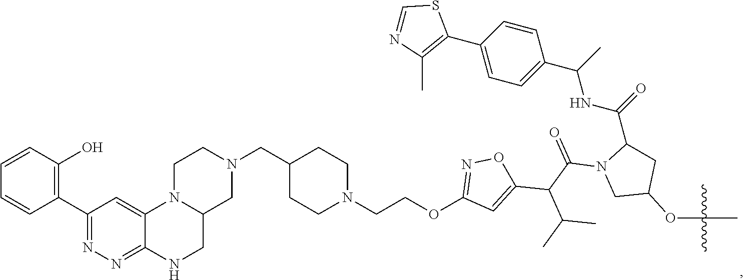 [SMILES: *OC1CC(C(=O)NC(C)c2ccc(-c3scnc3C)cc2)N(C(=O)C(c2cc(OCCN3CCC(CN4CCN5c6cc(-c7ccccc7O)nnc6NCC5C4)CC3)no2)C(C)C)C1]

wherein the wavy line indicates the point of covalent attachment to L.

In some embodiments, D has a structure of:

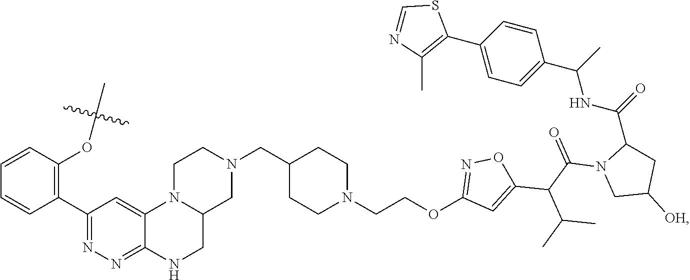 [SMILES: *Oc1ccccc1-c1cc2c(nn1)NCC1CN(CC3CCN(CCOc4cc(C(C(=O)N5CC(O)CC5C(=O)NC(C)c5ccc(-c6scnc6C)cc5)C(C)C)on4)CC3)CCN21]

wherein the wavy line indicates the point of covalent attachment to L.

In some embodiments, D has a structure of:

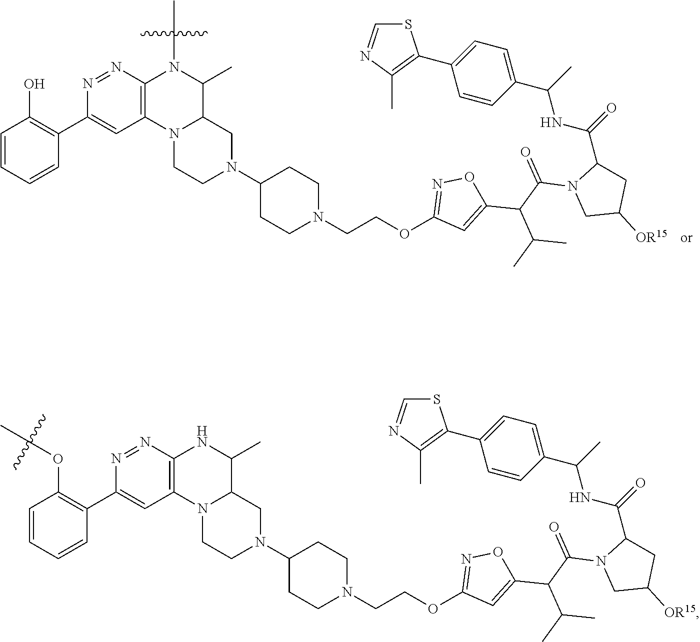

wherein R¹⁵ is hydrogen or —PO₃H₂and the wavy line indicates the point of covalent attachment to L.

In some embodiments, D has a structure of:

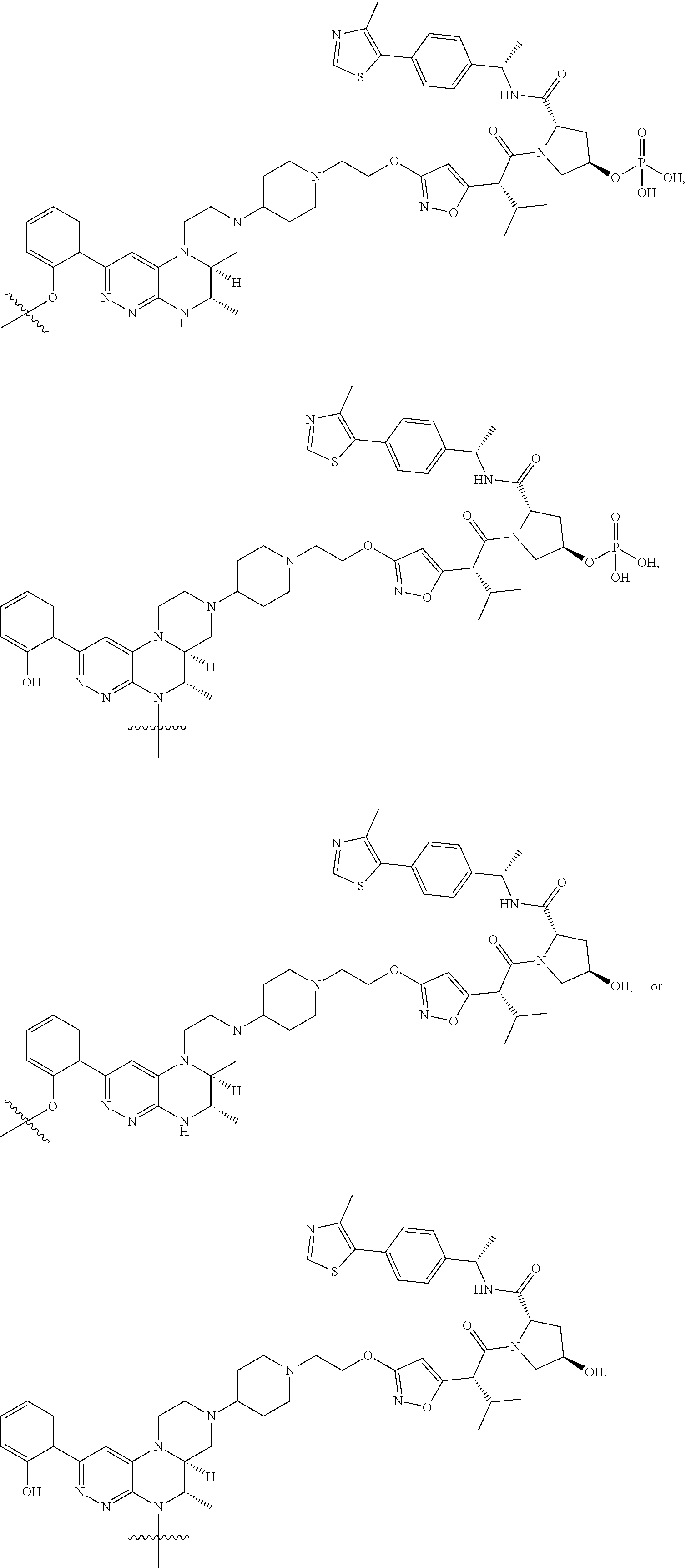

In some embodiments, ULM binds Cereblon E3 Ubiquitin Ligase. In some embodiments, ULM is a moiety having the Formula ULM-II-CRBN:

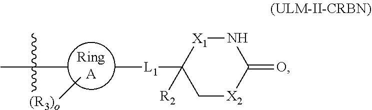

wherein
[symbol] is a point of attachment to PTM; Ring A is a monocyclic, bicyclic or tricyclic aryl, heteroaryl or heterocycle group,
L₁ is a bond, —O—, —S—, —NR^{a}—, —C(R^{a})₂—, or —C(O)NR^{a}—;
X₁ is a bond, —C(O)—, —C(S)—, —CH₂—, —CHCF₃—, SO₂—, —S(O), P(O)R^{b}—or —P(O)OR^{b}—;
X₂ is —C(R^{a})₂—, —NR^{a}— or —S—;
R₂ is H, deuterium, optionally substituted C₁₋₄ alkyl, C₁₋₄ alkoxyl, C₁₋₄haloalkyl, —CN, —OR^{a}, —OR^{b} or —SR^{b};
each R₃ is independently H, deuterium, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{e2}R^{d} —NR^{a}R^{c2}, —C(O)R^{b}, —OC(O)R^{a}, —C(O)OR^{a}, —C(O)NR^{e2}R^{d}, —S(O)R^{b}, —S(O)₂NR^{e2}R^{d}, —S(O)(═NR^{b})R^{b}, —SF₅, —P(O)R^{b}R^{b}, —P(O)(OR^{b})(OR^{b}), —B(OR^{d})(OR^{c2}) or —S(O)₂R^{b};
each R^{a} is independently H, deuterium, —C(O)R^{b}, —C(O)OR^{c2}, —C(O)NR^{c2}R^{d}, —C(═NR^{b})NR^{b}R^{c2}, —C(═NOR^{b})NR^{b}R^{c2}, —C(═NCN)NR^{b}R^{c2}, —P(OR^{c2})₂, —P(O)R^{e2}R^{b}, —P(O)OR^{e2}OR^{b}, —S(O)R^{b}, —S(O)NR^{c2}R^{d}, —S(O)₂R^{b}, —S(O)₂NR^{c2}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, deuterium, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c2} or R^{d} is independently H, deuterium, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c2} and R^{d}, together with the atom to which they are both attached, form a monocyclic or multicyclic heterocycloalkyl, or a monocyclic or multicyclic heterocyclo-alkenyl group; and
is 1, 2, 3, 4, or 5.

In some embodiments, ULM-II-CRBN is a compound of formula:

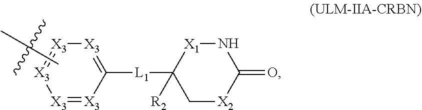

wherein each X₃ is independently N, N-oxide or CR₃ and at least one X₃ is N or N-oxide;
wherein [symbol] is a point of attachment to PTM; or

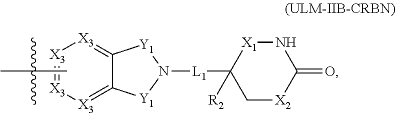

wherein each X₃ is independently N, N-oxide or CR₃;
wherein each Y₁ is independently —C(O)— or —C(R^{a})₂—and at least one Y₁ is —C(O)—; and
wherein [symbol] is a point of attachment to PTM; or

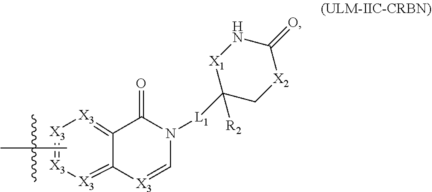

wherein each X₃ is independently N, N-oxide or CR₃ and wherein [symbol] is a point of attachment to PTM; or

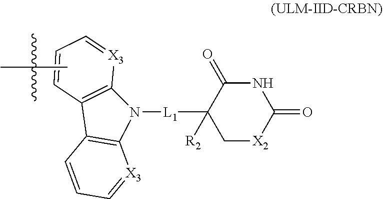

wherein each X₃ is independently N, N-oxide or CR₃ and wherein [symbol] is a point of attachment to PTM.

In some embodiments, ULM-II-CRBN is a compound of formula (ULM-IIA-CRBN). In some embodiments, ULM-II-CRBN is a compound of formula (ULM-IIB—CRBN). In some embodiments, ULM-II-CRBN is a compound of formula (ULM-IIC—CRBN). In some embodiments, ULM-II-CRBN is a compound of formula (ULM-IID-CRBN).

In some embodiments, D has a structure of:

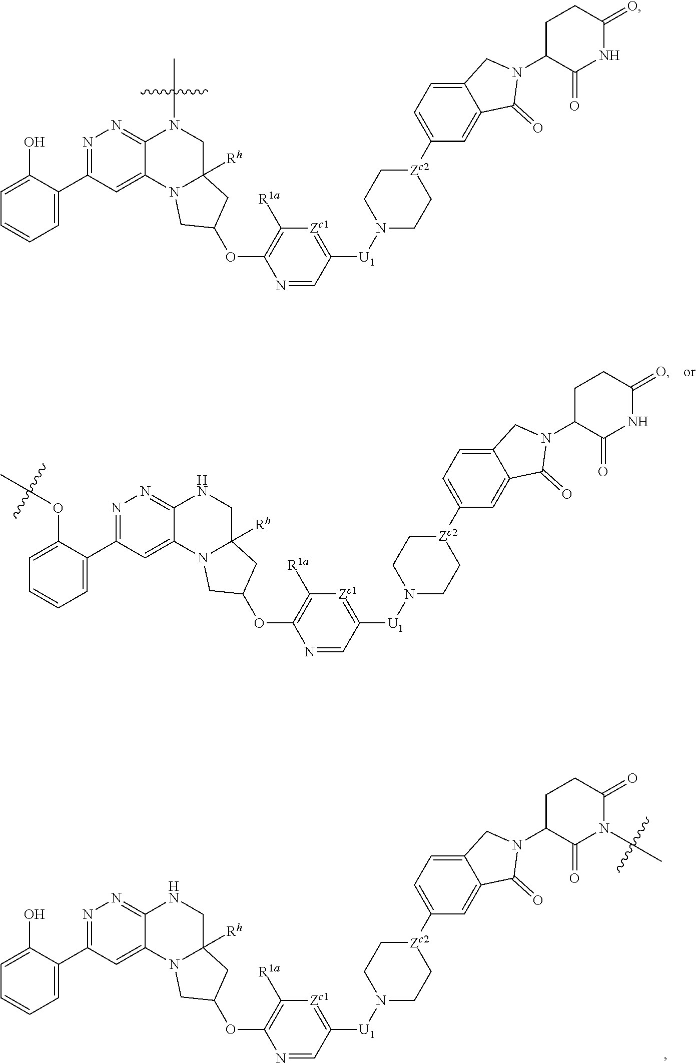

wherein
the wavy line indicates the point of covalent attachment to L;
R^{h} is C₁₋₃ alkyl;
R^{1a} is hydrogen, C₁₋₃ alkyl, or halogen;
Z^{c1} and Z^{c2} are each independently CH or N; and
U₁ is —CH₂— or —C(O)—.

In some embodiments, D has a structure of:

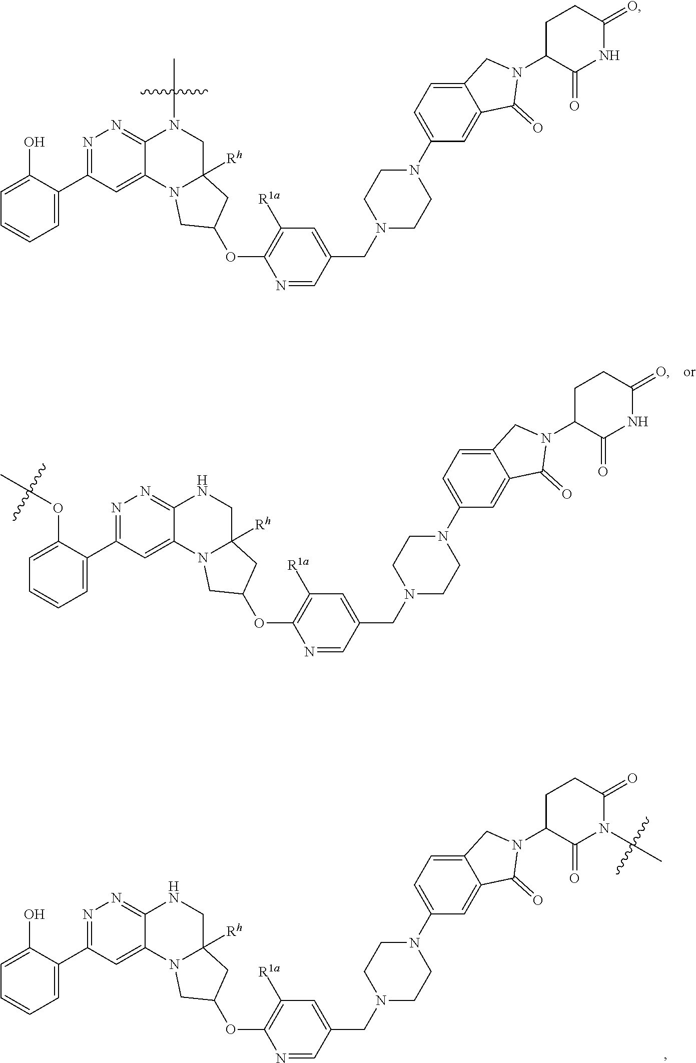

wherein
the wavy line indicates the point of covalent attachment to L;
R^{h} is methyl or ethyl; and
R^{1a} is hydrogen, methyl, or fluorine.

In some embodiments, D has a structure of:

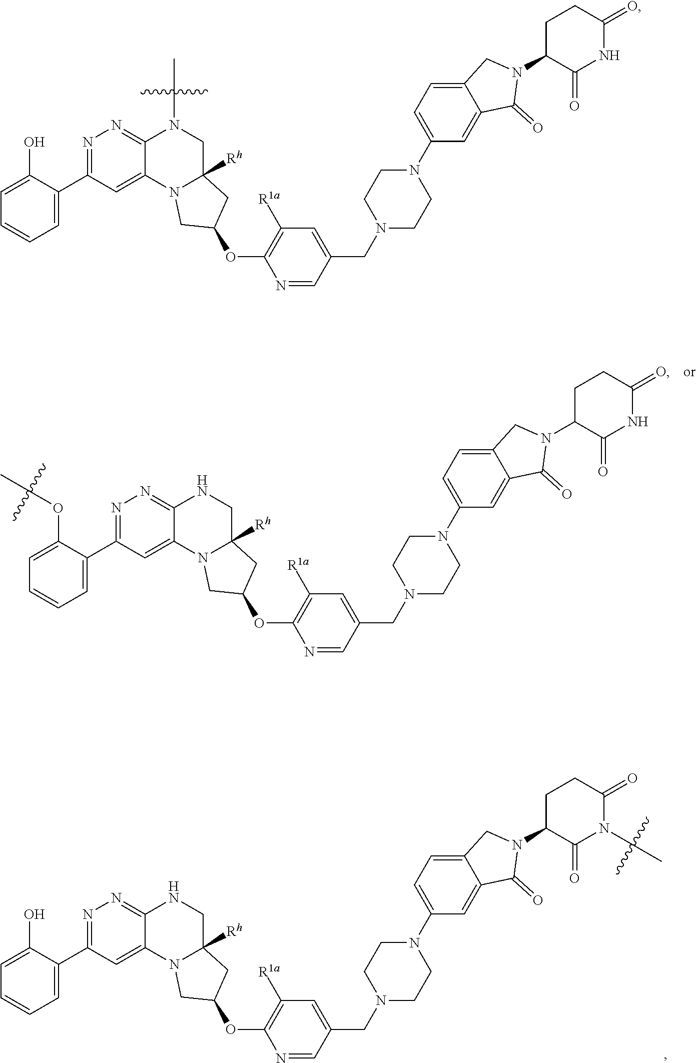

wherein
the wavy line indicates the point of covalent attachment to L;
R^{h} is methyl or ethyl; and
R^{1a} is hydrogen, methyl, or fluorine.

In some embodiments, the degrader compound of Formula (I) has the structure of:

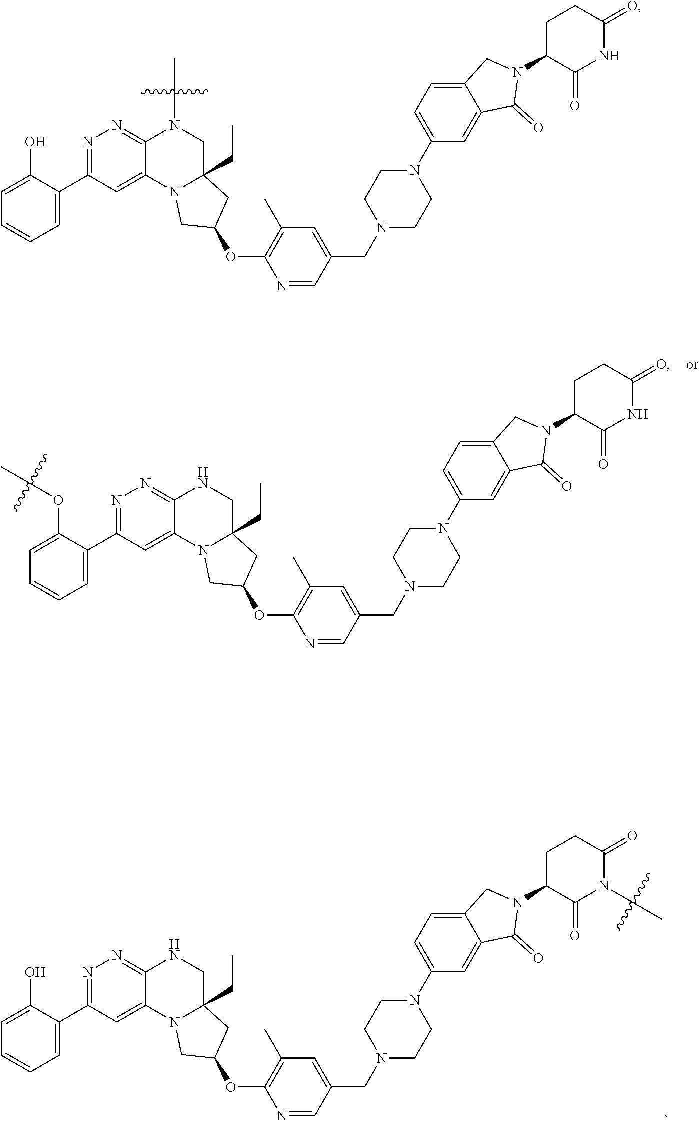 [SMILES: *N1C[C@]2(CC)C[C@@H](Oc3ncc(CN4CCN(c5ccc6c(c5)C(=O)N([C@H]5CCC(=O)NC5=O)C6)CC4)cc3C)CN2c2cc(-c3ccccc3O)nnc21 ; *Oc1ccccc1-c1cc2c(nn1)NC[C@]1(CC)C[C@@H](Oc3ncc(CN4CCN(c5ccc6c(c5)C(=O)N([C@H]5CCC(=O)NC5=O)C6)CC4)cc3C)CN21 ; *N1C(=O)CC[C@H](N2Cc3ccc(N4CCN(Cc5cnc(O[C@H]6CN7c8cc(-c9ccccc9O)nnc8NC[C@]7(CC)C6)c(C)c5)CC4)cc3C2=O)C1=O]

or a pharmaceutically acceptable salt or solvate thereof, wherein the wavy line indicates the point of attachment to L.

In some embodiments, the degrader compound of Formula (I) has the structure of:

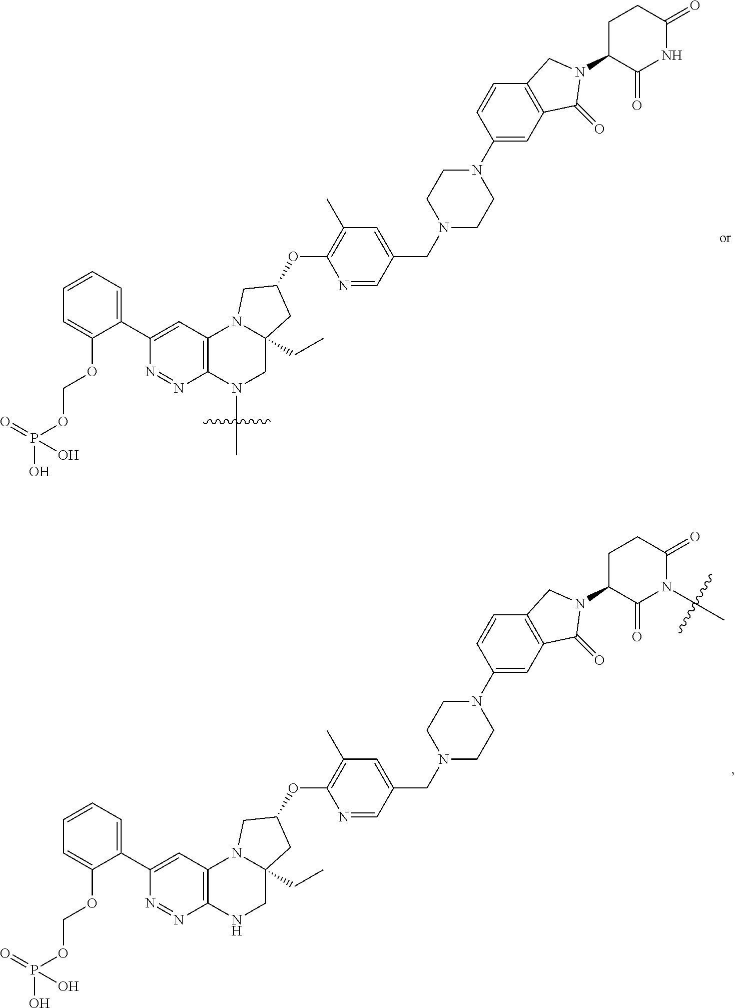 [SMILES: *N1C[C@]2(CC)C[C@@H](Oc3ncc(CN4CCN(c5ccc6c(c5)C(=O)N([C@H]5CCC(=O)NC5=O)C6)CC4)cc3C)CN2c2cc(-c3ccccc3OCOP(=O)(O)O)nnc21 ; *N1C(=O)CC[C@H](N2Cc3ccc(N4CCN(Cc5cnc(O[C@H]6CN7c8cc(-c9ccccc9OCOP(=O)(O)O)nnc8NC[C@]7(CC)C6)c(C)c5)CC4)cc3C2=O)C1=O]

or a pharmaceutically acceptable salt or solvate thereof, wherein the wavy line indicates the point of attachment to L.

In some embodiments, the degrader compound of Formula (I) has the structure of:

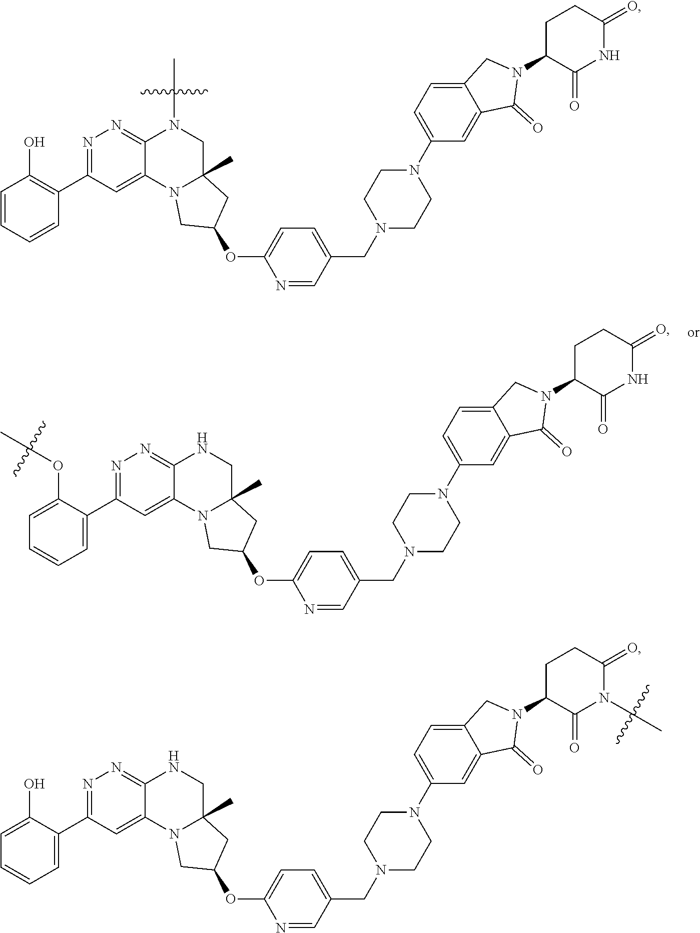 [SMILES: *N1C[C@]2(C)C[C@@H](Oc3ccc(CN4CCN(c5ccc6c(c5)C(=O)N([C@H]5CCC(=O)NC5=O)C6)CC4)cn3)CN2c2cc(-c3ccccc3O)nnc21 ; *Oc1ccccc1-c1cc2c(nn1)NC[C@]1(C)C[C@@H](Oc3ccc(CN4CCN(c5ccc6c(c5)C(=O)N([C@H]5CCC(=O)NC5=O)C6)CC4)cn3)CN21 ; *N1C(=O)CC[C@H](N2Cc3ccc(N4CCN(Cc5ccc(O[C@H]6CN7c8cc(-c9ccccc9O)nnc8NC[C@]7(C)C6)nc5)CC4)cc3C2=O)C1=O]

or a pharmaceutically acceptable salt or solvate thereof, wherein the wavy line indicates the point of attachment to L.

In some embodiments, the Antibody Drug Conjugate compound has a formula of:

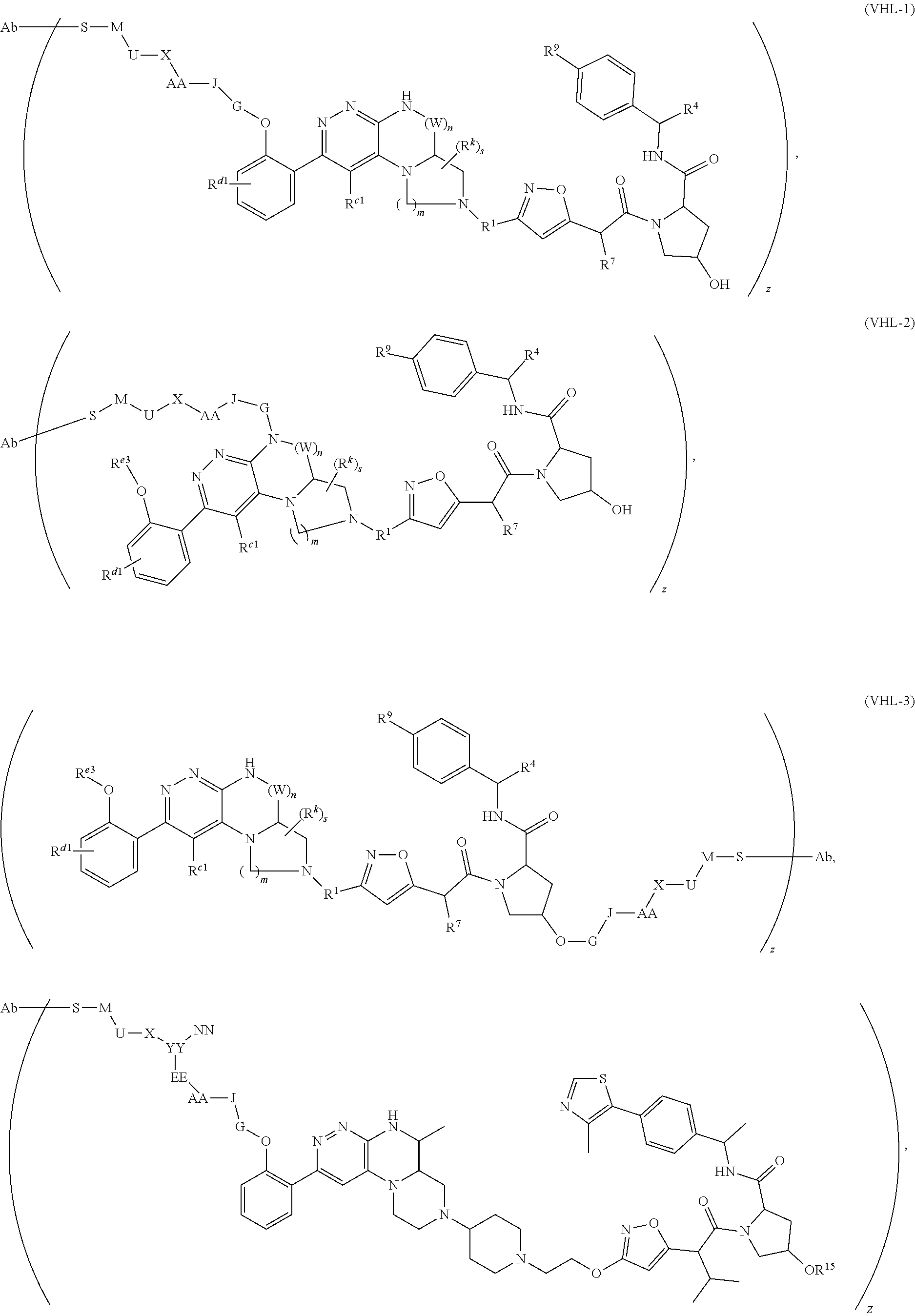

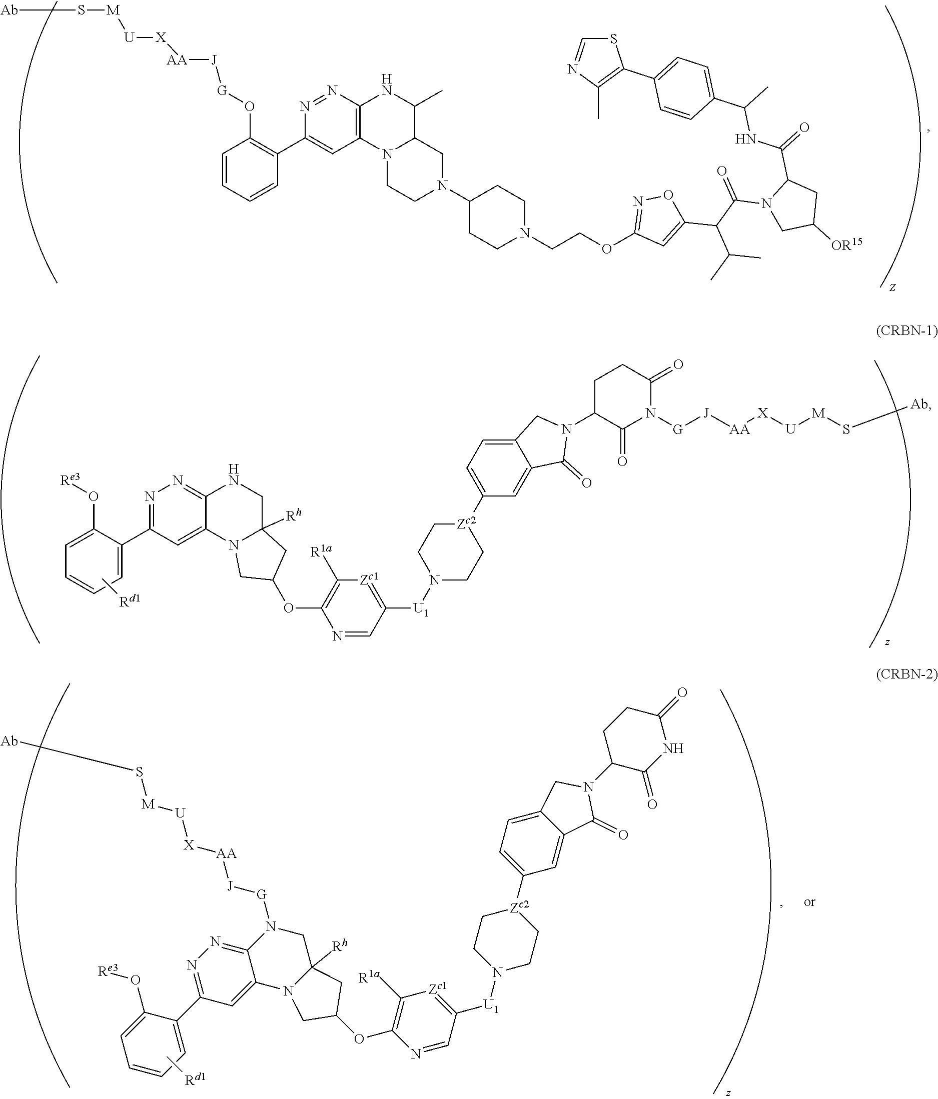

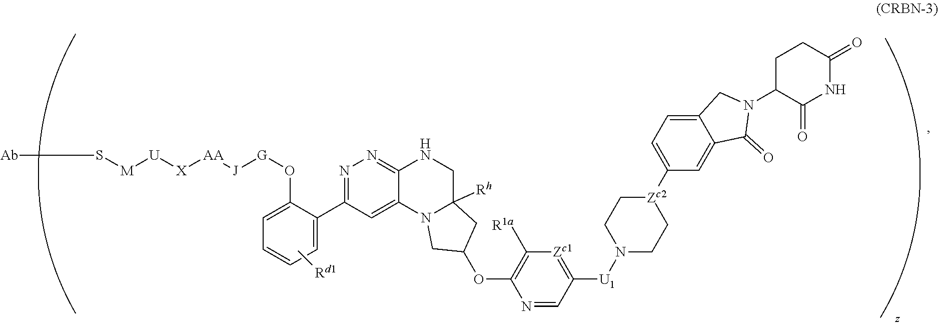

wherein R^{h} is methyl or ethyl; R^{1a} is hydrogen, methyl, or fluorine; each of Z^{c1} and Z^{c2} is M independently CH or N; and U₁ is —CH₂— or —C(O)—.

In some embodiments, ULM-II-CRBN is a compound of formula (ULM-IID-CRBN). In some embodiments, the Antibody Drug Conjugate compound has a formula of (VHL-1). In some embodiments, the Antibody Drug Conjugate compound has a formula of (VHL-2). In some embodiments, the Antibody Drug Conjugate compound has a formula of (VHL-3). In some embodiments, the Antibody Drug Conjugate compound has a formula of (CRBN-1). In some embodiments, the Antibody Drug Conjugate compound has a formula of (CRBN-2). In some embodiments, the Antibody Drug Conjugate compound has a formula of (CRBN-3).

In some embodiments, R^{e3} is H. In some embodiments, R^{c1} is H. In some embodiments, R^{d1} is H. In some embodiments, R^{h} is ethyl. In some embodiments, R^{h} is methyl. In some embodiments, R^{1a} is H. In some embodiments, R^{1a} is methyl. In some embodiments, R^{1a} is F. In some embodiments, Z^{c1} is CH. In some embodiments, Z^{c1} is N. In some embodiments, Z^{c2} is N. In some embodiments, Z^{c2} is CH. In some embodiments, U₁ is —CH₂—. In some embodiments, U₁ is —C(O)—. In some embodiments, R^{c1} is H, R^{d1} is H, and R^{h} is methyl. In some embodiments, R^{c1} is H, R^{d1} is H, and R^{h} is ethyl. In some embodiments, Z^{c1} is CH and Z^{c2} is N. In some embodiments, Z^{c1} is CH and Z^{c2} is CH. In some embodiments, Z^{c1} is CH and Z^{c2} is N. In some embodiments, Z^{c1} is N and Z^{c2} is CH. In some embodiments, Z^{c1} is N and Z^{c2} is N.

In some embodiments, AA is present. In some embodiments, subscript c is an integer ranging between 1 and 12. In some embodiments, each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine. In some embodiments, AA is present, subscript c is an integer ranging between 1 and 12, and each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine. In some embodiments, each R^{a1} is independently —COOH or —NH₂. In some embodiments, each R^{a1} is independently —COOH. In some embodiments, each R^{a1} is independently —NH₂. In some embodiments, each R^{a1} is an independently selected side chain of an amino acid, wherein each amino acid is selected from the group consisting of alanine, valine, glutamic acid, citrulline, tryptophan, glycine, phenylalanine, and lysine.

In some embodiments, the Antibody Drug Conjugate compound is represented by the structure of

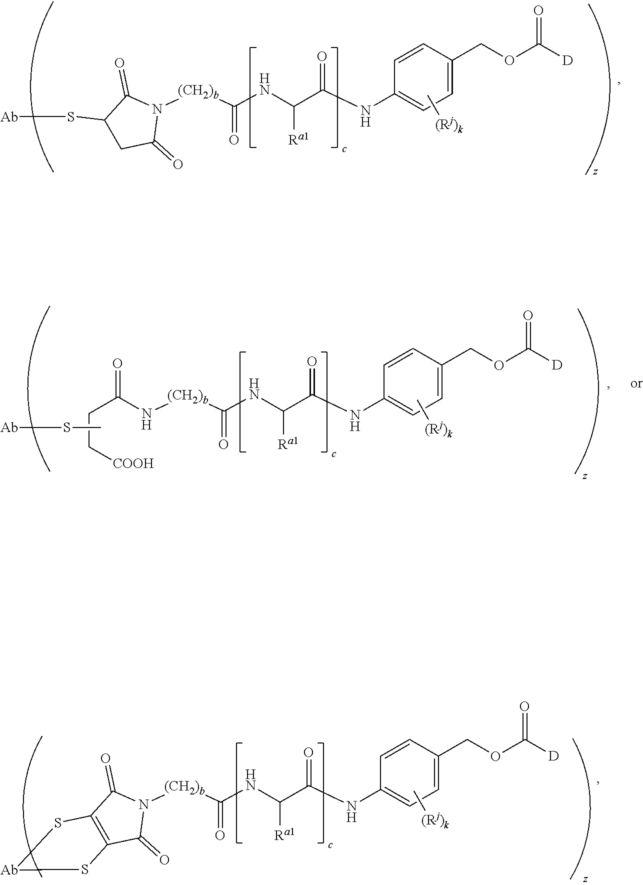

wherein S is a sulfur atom of the Ab; subscript b is an integer ranging from 1 to 5; subscript c is an integer ranging from 1 to 5; each R^{a1} is independently the side chain of valine, glutamic acid, tryptophan, glycine, citrulline, lysine, alanine, or phenylalanine; each R^{j} is independently fluorine, chlorine, methyl, or —C(O)NHCH₃; and subscript k is an integer ranging from 0 to 4.

In some embodiments, the Antibody Drug Conjugate compound is represented by the structure of

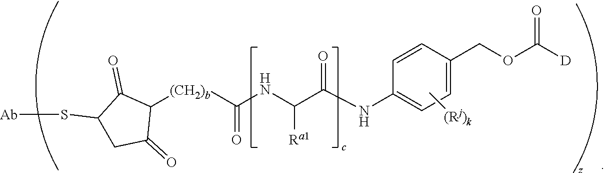

In some embodiments, the Antibody Drug Conjugate compound is represented by the structure of

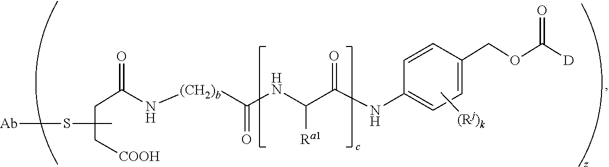

In some embodiments, the Antibody Drug Conjugate compound is represented by the structure of

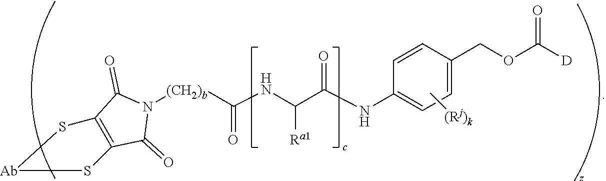

In some embodiments, the Antibody Drug Conjugate compound is represented by the structure of

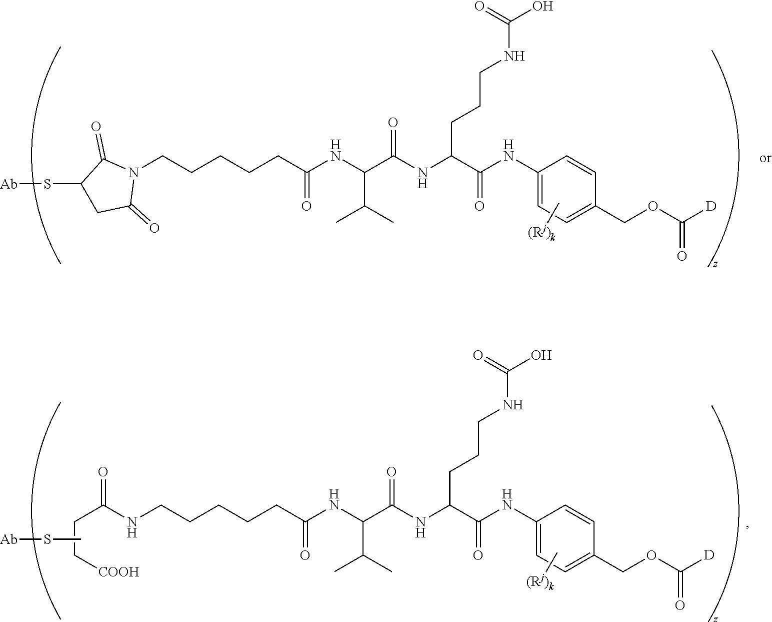

wherein R^{j} is methyl, —F, —Cl, or —C(O)NHCH₃, and subscript k is 0 or 1.

In some embodiments, the Antibody Drug Conjugate compound is represented by the structure of

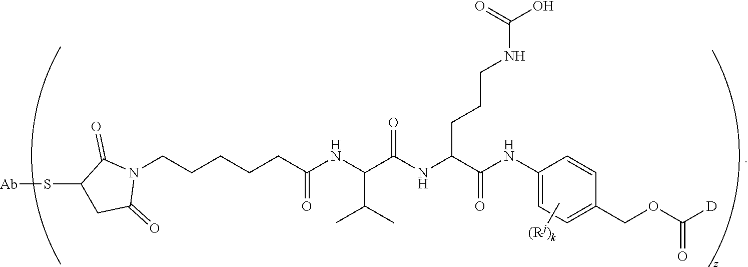

In some embodiments, the Antibody Drug Conjugate compound is represented by the structure of

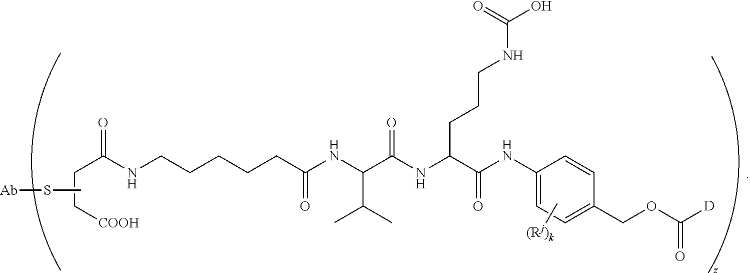

In some embodiments, the Antibody Drug Conjugate compound is represented by the structure of

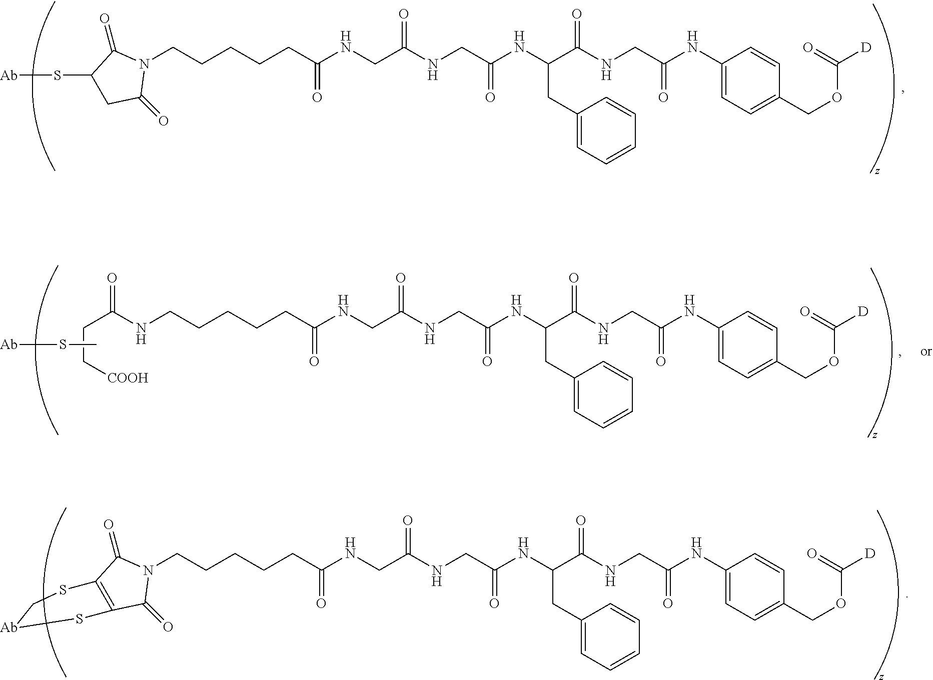

In some embodiments, the Antibody Drug Conjugate compound is represented by the structure of

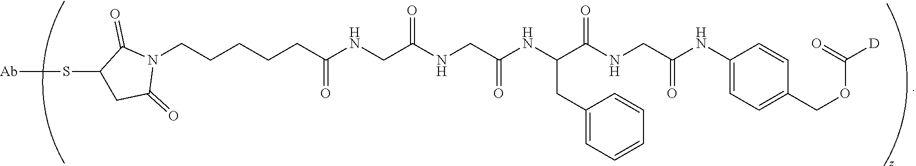

In some embodiments, the Antibody Drug Conjugate compound is represented by the structure of

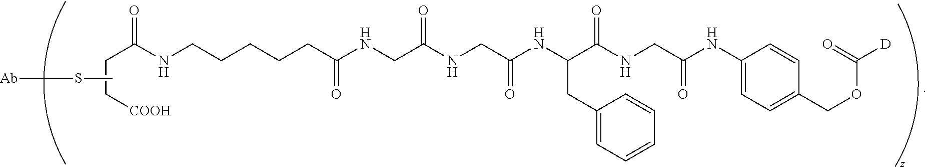

In some embodiments, the Antibody Drug Conjugate compound is represented by the structure of

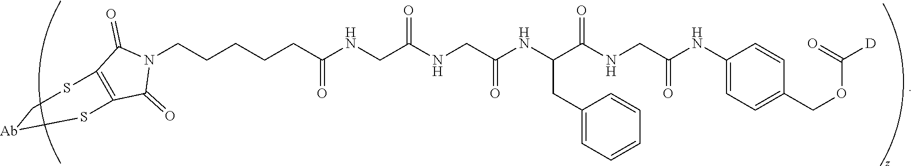

In some embodiments, the Antibody Drug Conjugate compound is represented by the structure of

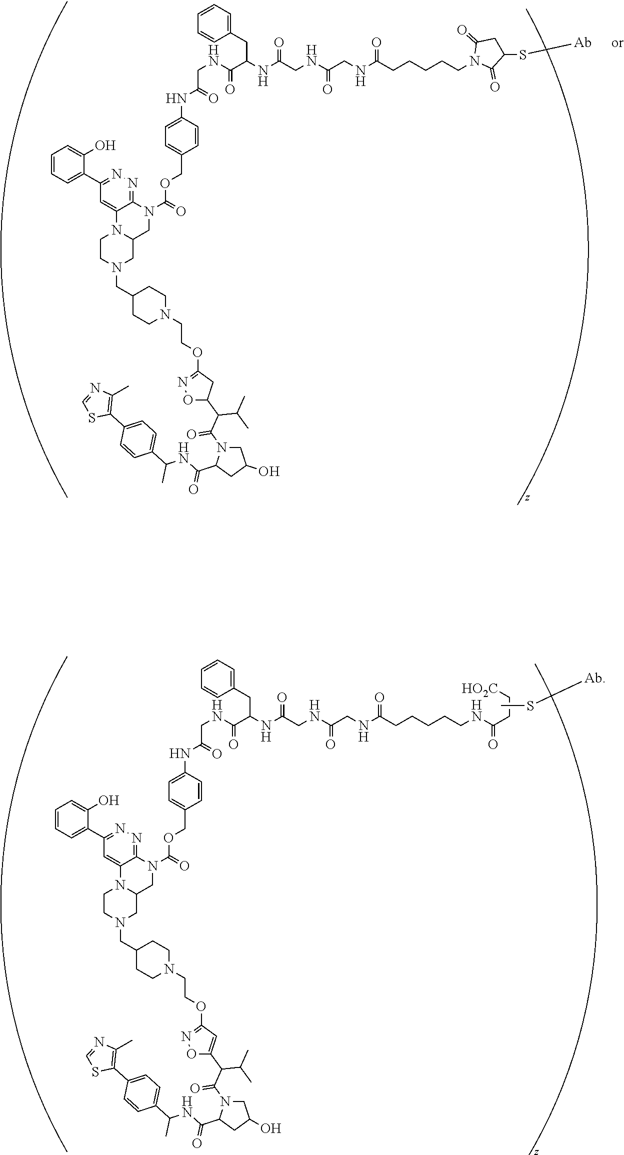

In some embodiments, the Antibody Drug Conjugate compound is represented by the structure of

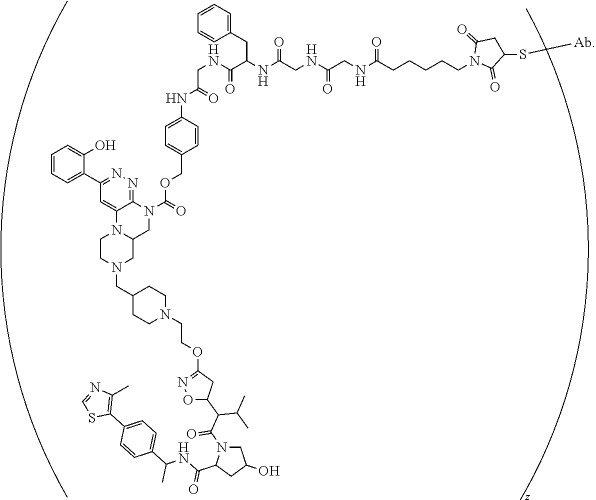

In some embodiments, the Antibody Drug Conjugate compound is represented by the structure of

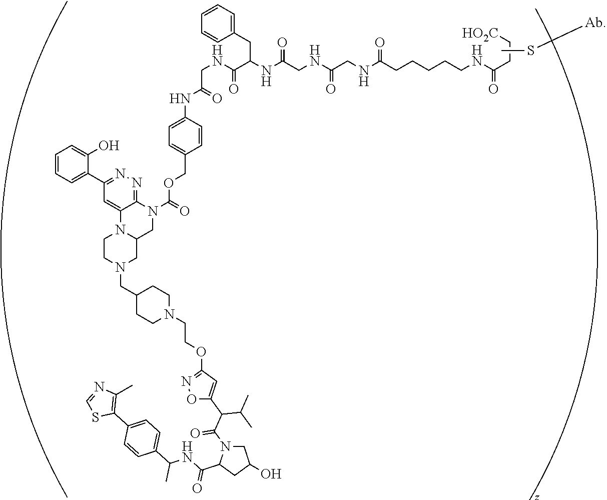

In some embodiments, the Antibody Drug Conjugate compound is represented by the structure of

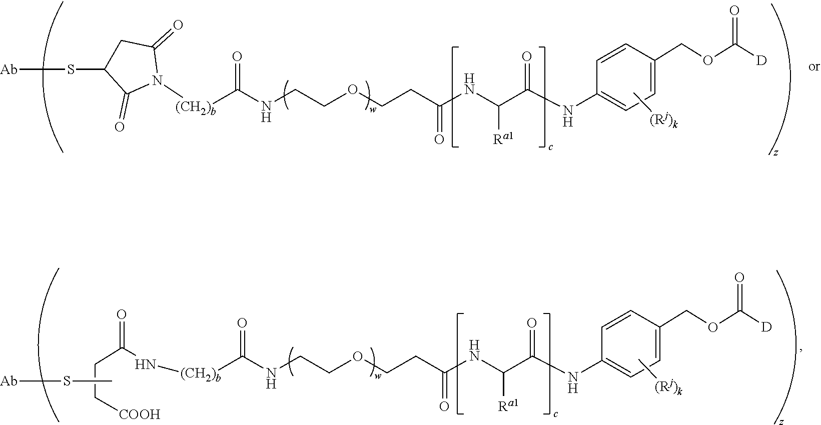

wherein S is a sulfur atom of the Ab; subscript b is an integer ranging from 1 to 5; subscript w is an integer ranging from 1 to 8; subscript c is an integer ranging from 1 to 3; each R^{a1} is independently the side chain of valine, glutamic acid, tryptophan, glycine, citrulline, lysine, alanine, or phenylalanine; each R^{j} is independently fluorine, chlorine, methyl, or —C(O)NHCH₃; and subscript k is an integer ranging from 0 to 4.

In some embodiments, the Antibody Drug Conjugate compound is represented by the structure of

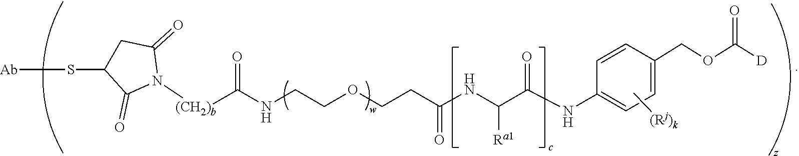

In some embodiments, the Antibody Drug Conjugate compound is represented by the structure of

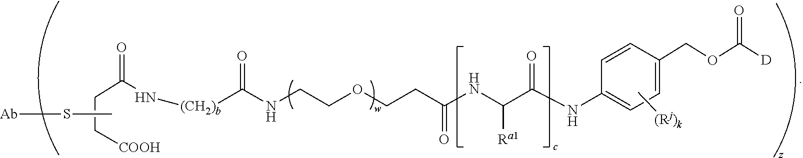

In some embodiments, the Antibody Drug Conjugate compound is represented by the structure of

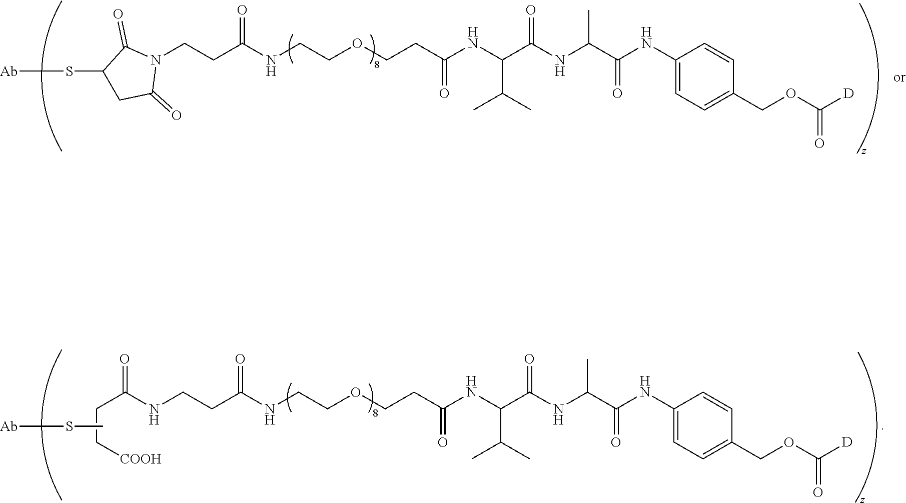

In some embodiments, the Antibody Drug Conjugate compound is represented by the structure of

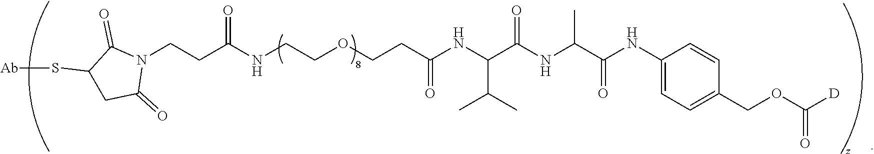

In some embodiments, the Antibody Drug Conjugate compound is represented by the structure of

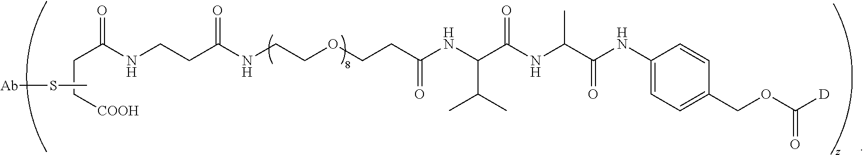

In some embodiments, the Antibody Drug Conjugate compound is represented by the structure of

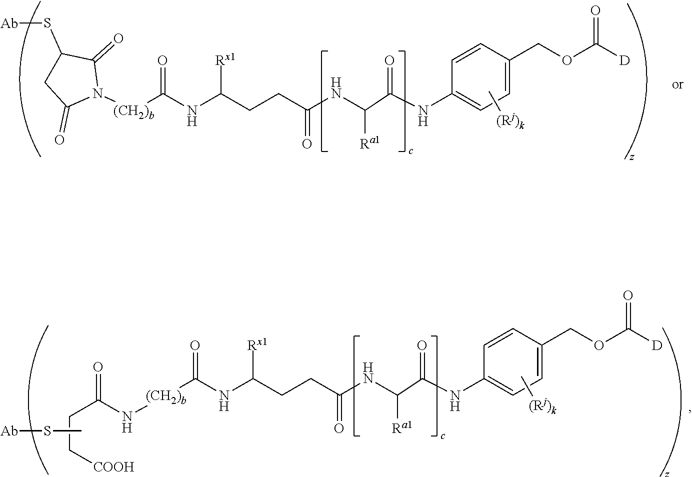

wherein S is a sulfur atom of the Ab; subscript b is an integer ranging from 1 to 5; subscript c is an integer ranging from 1 to 3; R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; each R^{a1} is independently the side chain of valine, glutamic acid, tryptophan, glycine, citrulline, lysine, alanine, or phenylalanine; each R^{j} is independently fluorine, chlorine, methyl, or —C(O)NHCH₃; and subscript k is an integer ranging from 0 to 4.

In some embodiments, the Antibody Drug Conjugate compound is represented by the structure of

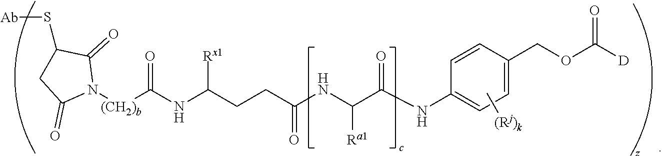

In some embodiments, the Antibody Drug Conjugate compound is represented by the structure of

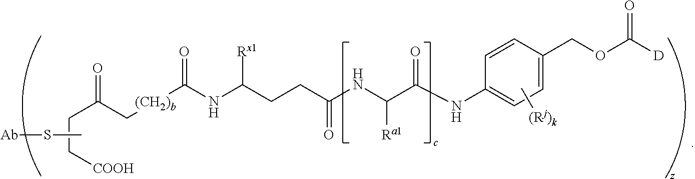

In some embodiments, R^{x1} is hydrogen. In some embodiments, R^{x1} is hydrogen, —COOH, or —C(O)NHCH₃. In some embodiments, R^{x1} is —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl. In some embodiments, each R^{j} is independently fluorine or chlorine.

In some embodiments, the Antibody Drug Conjugate compound is represented by the structure of

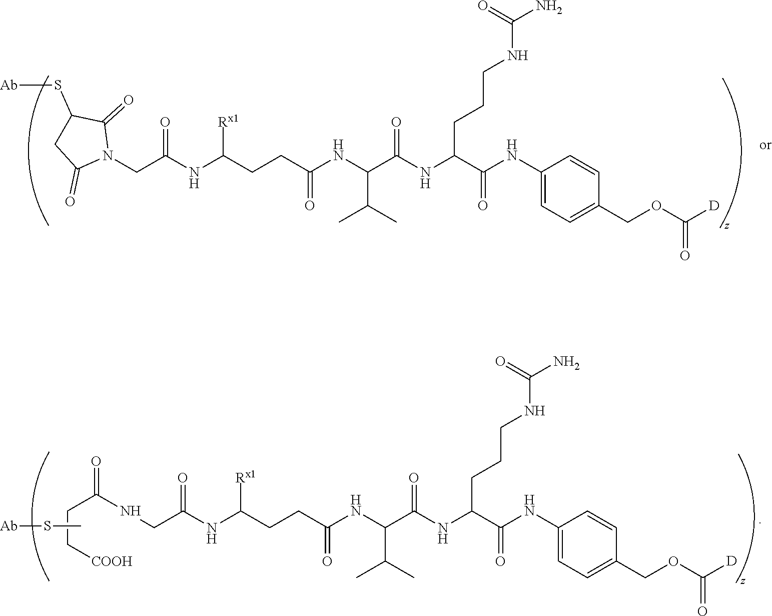

In some embodiments, the Antibody Drug Conjugate compound is represented by the structure of

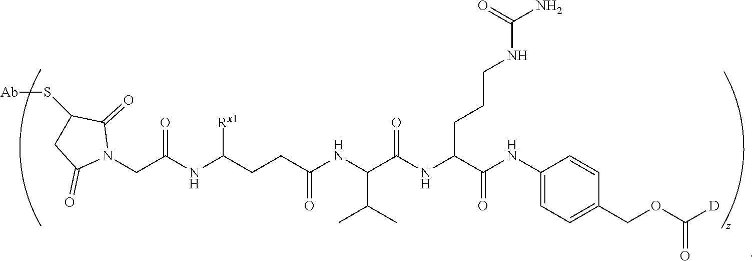

In some embodiments, the Antibody Drug Conjugate compound is represented by the structure of

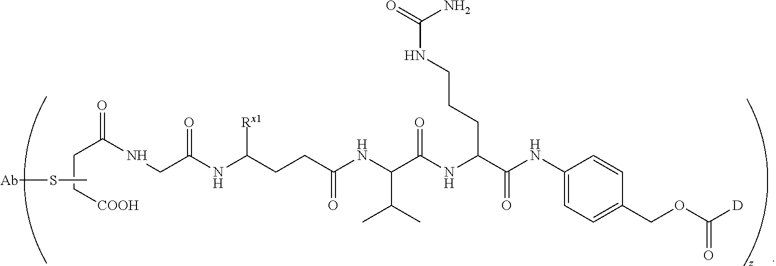

In some embodiments, R^{x1} is

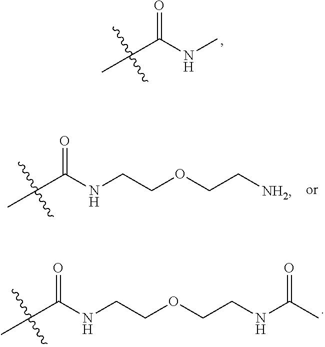

In some embodiments, R^{x1} is

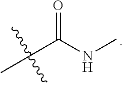 [SMILES: *C(=O)NC]

In some embodiments, R^{x1} is

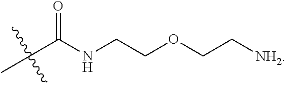 [SMILES: *C(=O)NCCOCCN]

In some embodiments, R^{x1} is

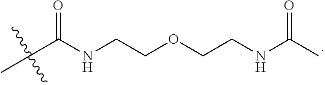 [SMILES: *C(=O)NCCOCCNC(C)=O]

In some embodiments, the Antibody Drug Conjugate compound is represented by the structure of

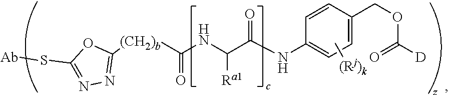

S is a sulfur atom of the Ab; subscript b is an integer ranging from 1 to 5; subscript c is 1 or 2; each R^{a1} is independently the side chain of valine or citrulline; each R^{j} is independently fluorine, chlorine, methyl, or —C(O)NHCH₃; and subscript k is an integer ranging from 0 to 4. In some embodiments, each R^{j} is independently fluorine or chlorine. In some embodiments, subscript k is 0.

In some embodiments, the Antibody Drug Conjugate compound is represented by the structure of

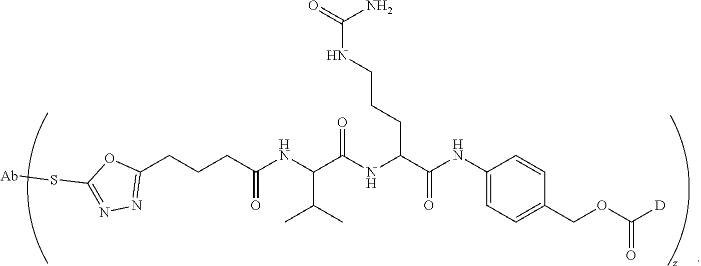

In some embodiments, the Antibody Drug Conjugate compound is represented by the structure of

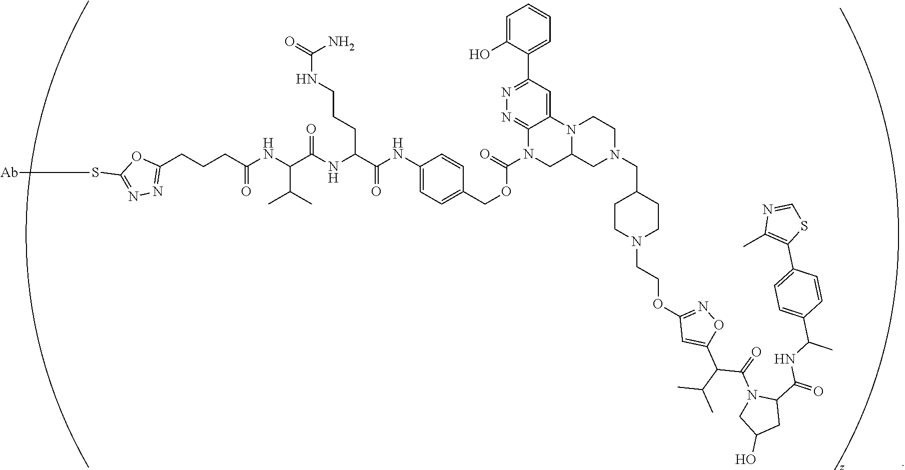

In some embodiments, the Antibody Drug Conjugate compound is represented by the structure of

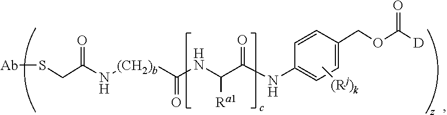

wherein S is a sulfur atom of the Ab; subscript b is an integer ranging from 1 to 5; subscript c is 1 or 2; each R^{a1} is independently the side chain of valine or citrulline; each R^{j} is independently fluorine, chlorine, methyl, or —C(O)NHCH₃; and subscript k is an integer ranging from 0 to 4. In some embodiments, each R^{j} is independently fluorine or chlorine. In some embodiments, subscript k is 0.

In some embodiments, the Antibody Drug Conjugate compound is represented by the structure of Z

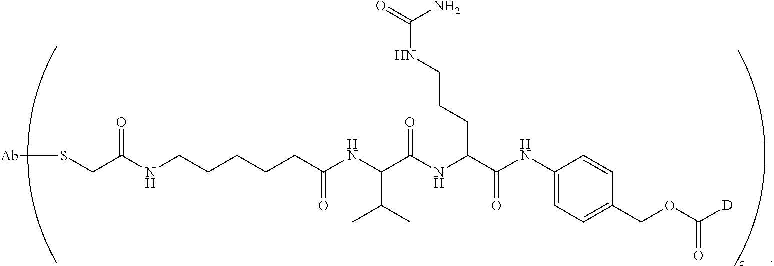

In some embodiments, the Antibody Drug Conjugate compound is represented by the structure of z

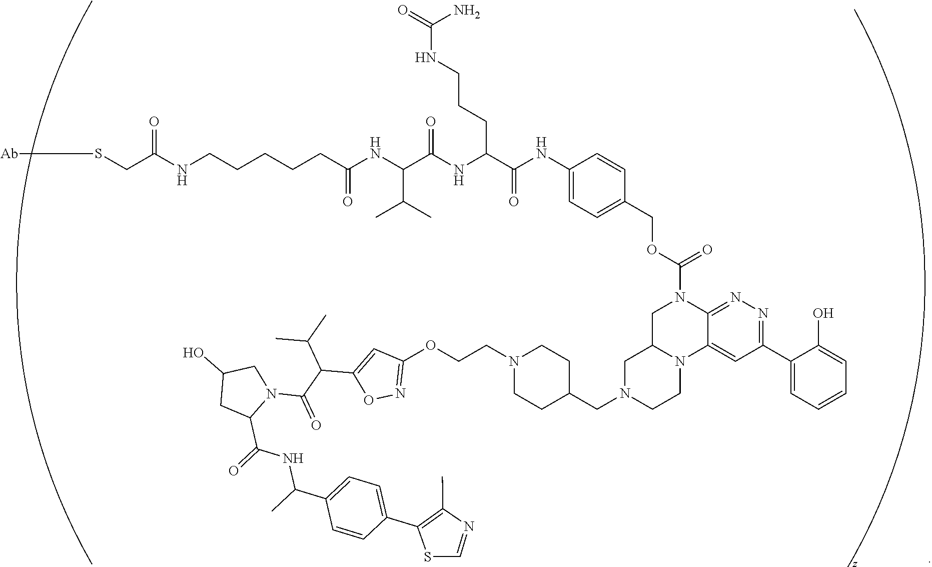

In some embodiments, the Antibody Drug Conjugate compound is represented by the structure of

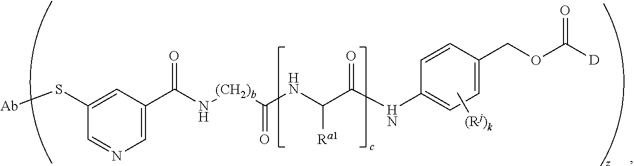

wherein S is a sulfur atom of the Ab; subscript b is an integer ranging from 1 to 5; subscript c is an integer ranging from 1 to 4; each R^{a1} is independently the side chain of valine or citrulline; each R^{j} is independently fluorine, chlorine, methyl, or —C(O)NHCH₃; and subscript k is an integer ranging from 0 to 4.

In some embodiments, the Antibody Drug Conjugate compound is represented by the structure of

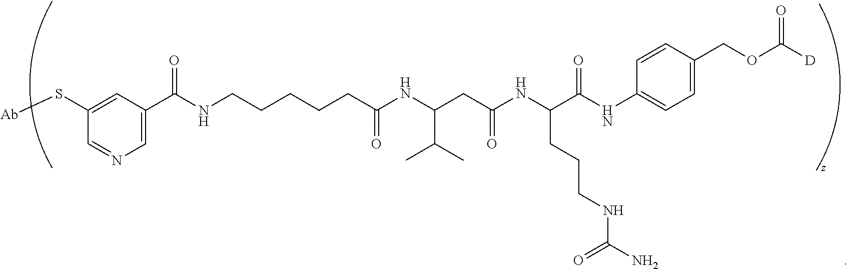

In some embodiments, the Antibody Drug Conjugate compound is represented by the structure of

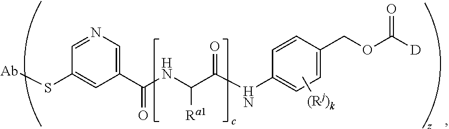

wherein S is a sulfur atom of the Ab; subscript c is an integer ranging from 1 to 4; each R^{a1} is independently the side chain of valine or citrulline; each R^{j} is independently fluorine, chlorine, methyl, or —C(O)NHCH₃; and subscript k is an integer ranging from 0 to 4.

In some embodiments, the Antibody Drug Conjugate compound is represented by the structure of z

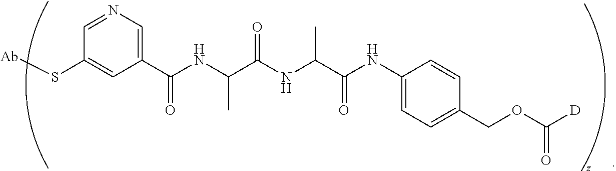

In some embodiments, the Antibody Drug Conjugate compound is represented by the structure of

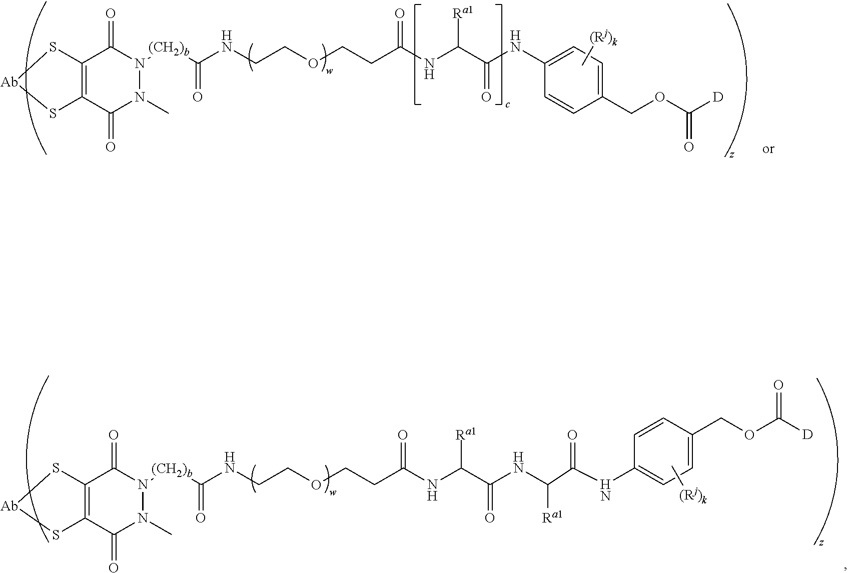

wherein S is a sulfur atom of the Ab; subscript c is an integer ranging from 1 to 4; each R^{a1} is independently the side chain of valine or citrulline; each R^{j} is independently fluorine, chlorine, methyl, or —C(O)NHCH₃; and subscript k is an integer ranging from 0 to 4. In some embodiments, subscript k is 0. In some embodiments, subscript k is 1.

In some embodiments, the Antibody Drug Conjugate compound is represented by the structure of

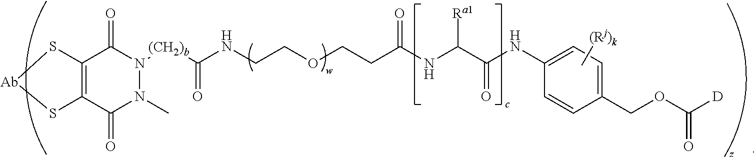

In some embodiments, the Antibody Drug Conjugate compound is represented by the structure of

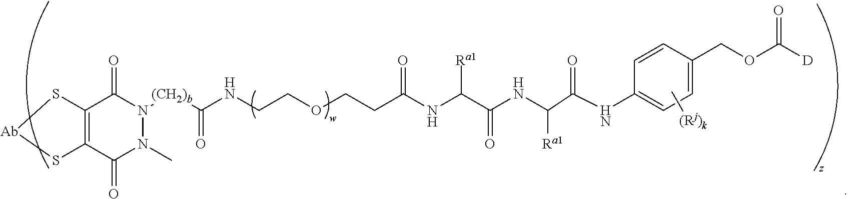

In some embodiments, the Antibody Drug Conjugate compound is represented by the structure of

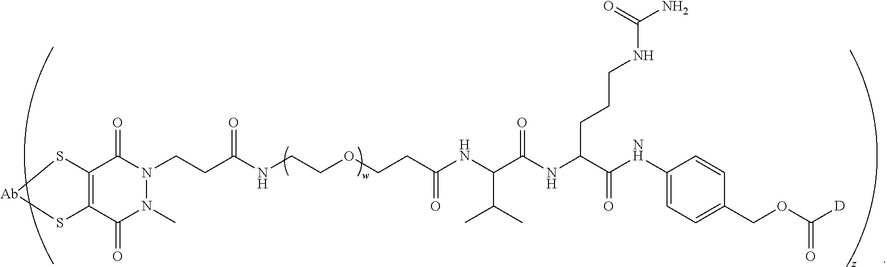

In some embodiments, the Antibody Drug Conjugate compound is represented by the structure of

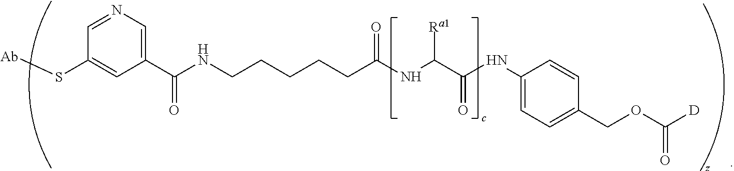

In some embodiments, the Antibody Drug Conjugate compound is represented by the structure of

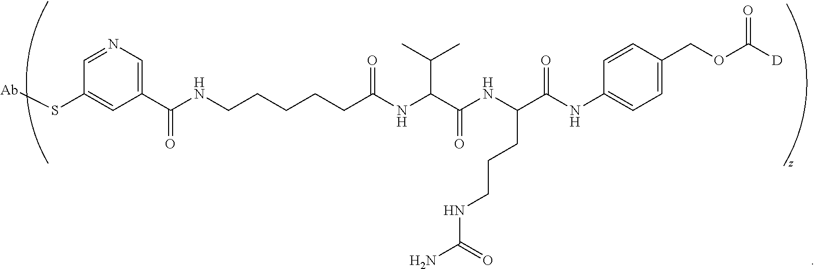

In some embodiments, the Antibody Drug Conjugate compound is represented by the structure of

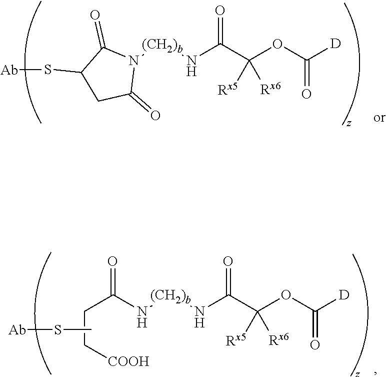

wherein subscript b is an integer ranging from 1 to 5; R^{x5} is hydrogen or C₁-C₆ alkyl; and R^{x6} is hydrogen, C₁-C₆ alkyl, or

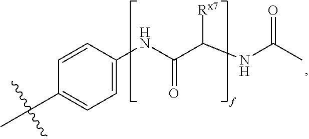

wherein subscript f is an integer ranging from 0 to 4 and each R^{x7} is independently hydrogen, C₁-C₆ alkyl, or an independently selected side chain of an amino acid. In some embodiments, b is 5. In some embodiments, R^{x5} is hydrogen. In some embodiments, R^{x6} is hydrogen. In some embodiments, the Antibody Drug Conjugate compound is represented by the structure of

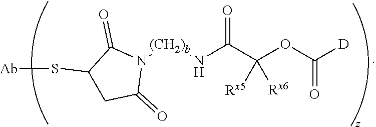

In some embodiments, the Antibody Drug Conjugate compound is represented by the structure of

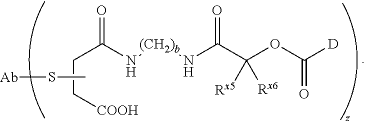

In some embodiments, the Antibody Drug Conjugate compound is represented by the structure of or

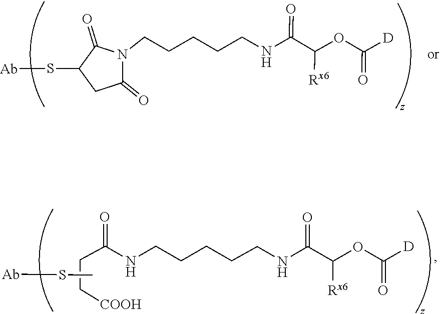

wherein R^{x6} is

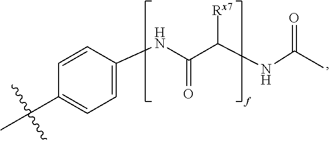

wherein subscript f is an integer ranging from 0 to 4 and each R^{x7} is
independently hydrogen, C₁-C₆ alkyl, or an independently selected side chain of an amino acid. In some embodiments, the Antibody Drug Conjugate compound is represented by the structure of

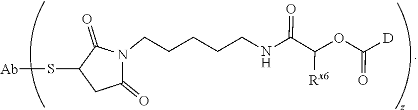

In some embodiments, the Antibody Drug Conjugate compound is represented by the structure of

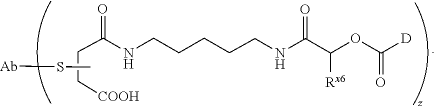

In some embodiments, R^{x6} is

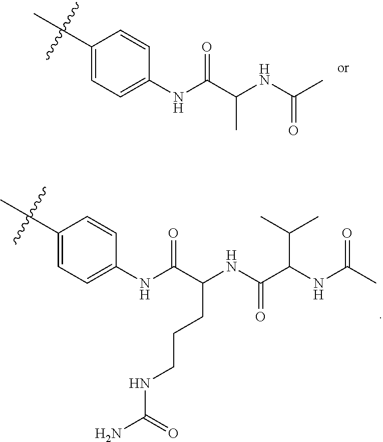 [SMILES: *c1ccc(NC(=O)C(C)NC(C)=O)cc1 ; *c1ccc(NC(=O)C(CCCNC(N)=O)NC(=O)C(NC(C)=O)C(C)C)cc1]

In some embodiments, R^{x6} is

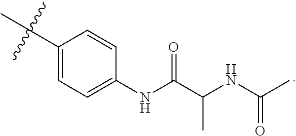 [SMILES: *c1ccc(NC(=O)C(C)NC(C)=O)cc1]

In some embodiments, R^{x6} is

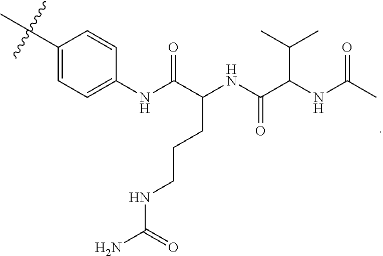 [SMILES: *c1ccc(NC(=O)C(CCCNC(N)=O)NC(=O)C(NC(C)=O)C(C)C)cc1]

In some embodiments, the Antibody Drug Conjugate compound is represented by the structure of:

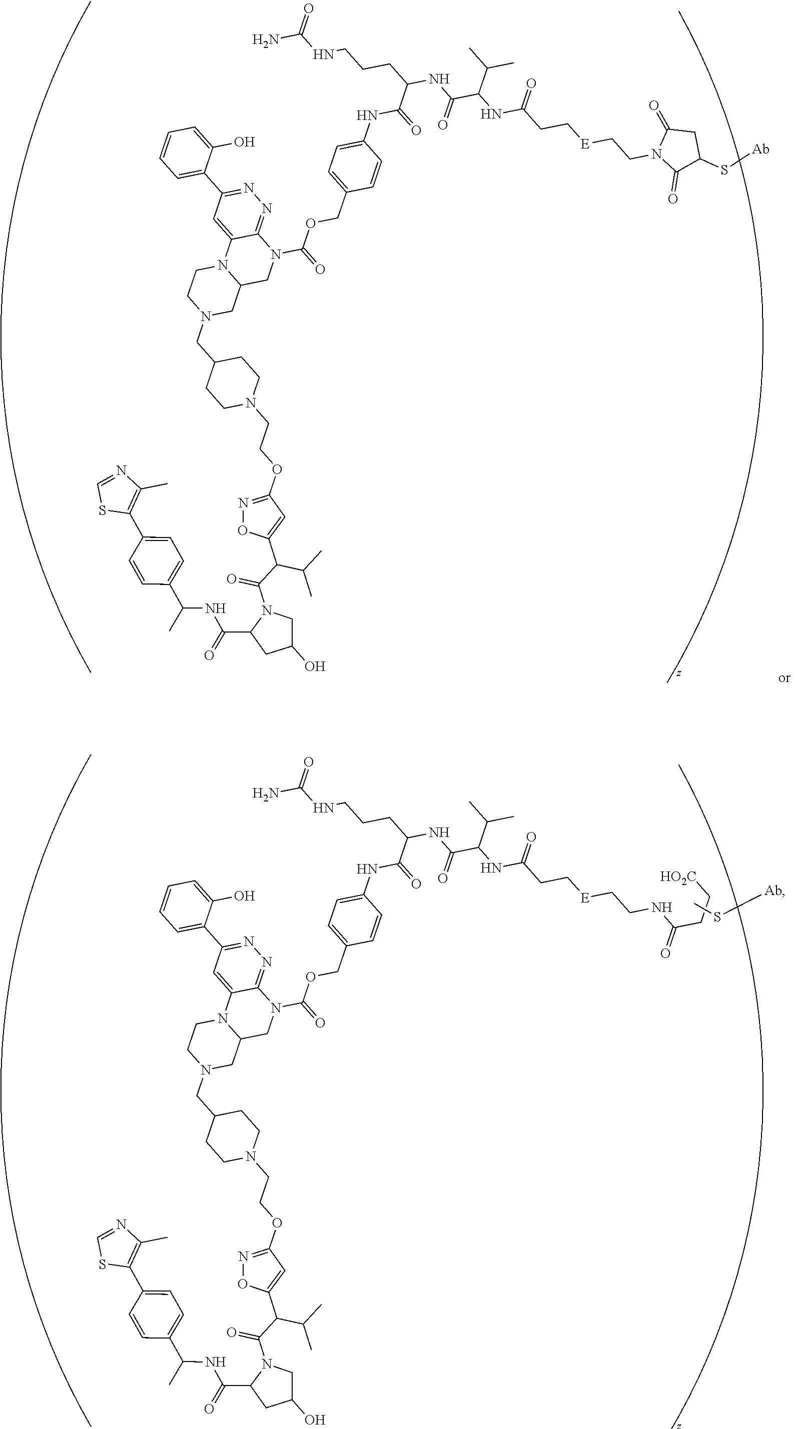

wherein E is —CH₂— or —O—. In some embodiments, E is —CH₂—. In some embodiments, E is —O—.

In some embodiments, the Antibody Drug Conjugate compound is represented by the structure of:

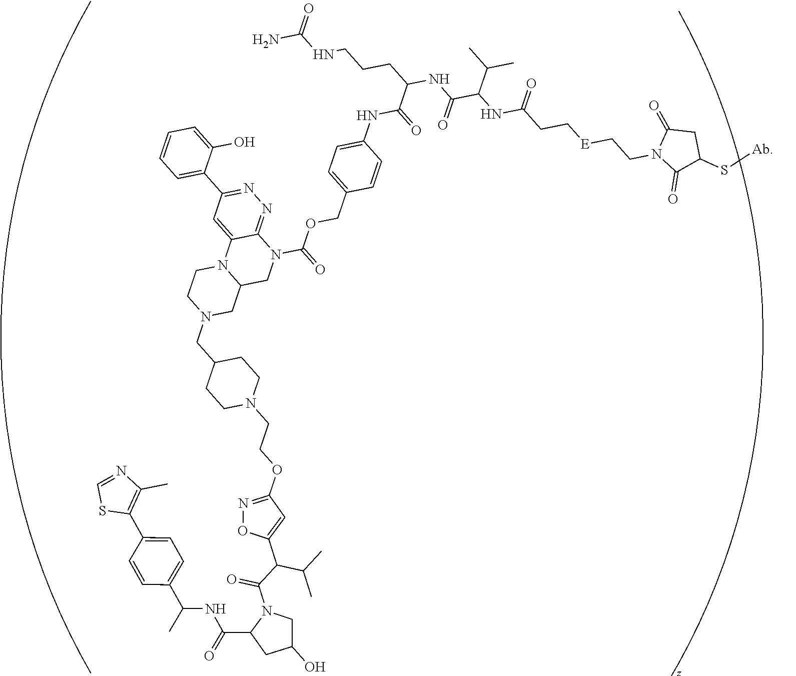

In some embodiments, the Antibody Drug Conjugate compound is represented by the structure of:

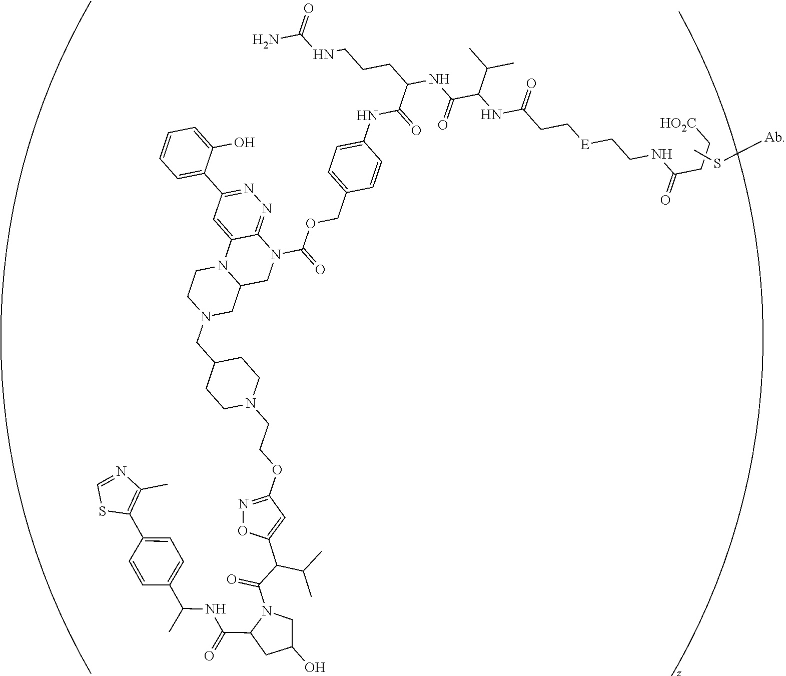

In some embodiments, the Antibody Drug Conjugate compound is represented by the structure of:

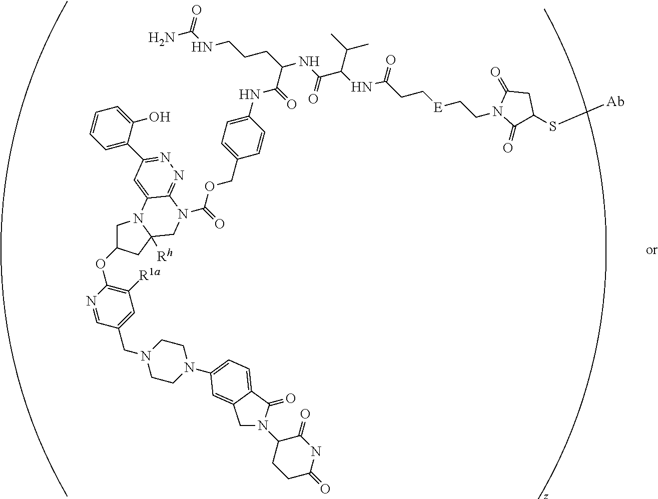

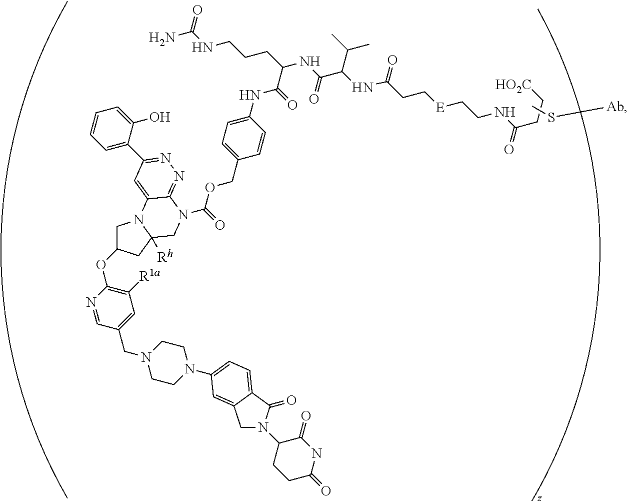

wherein E is —CH₂— or —O—; R^{h} is hydrogen, methyl, or ethyl; and R^{1a} is hydrogen or methyl.

In some embodiments, the Antibody Drug Conjugate compound is represented by the structure of

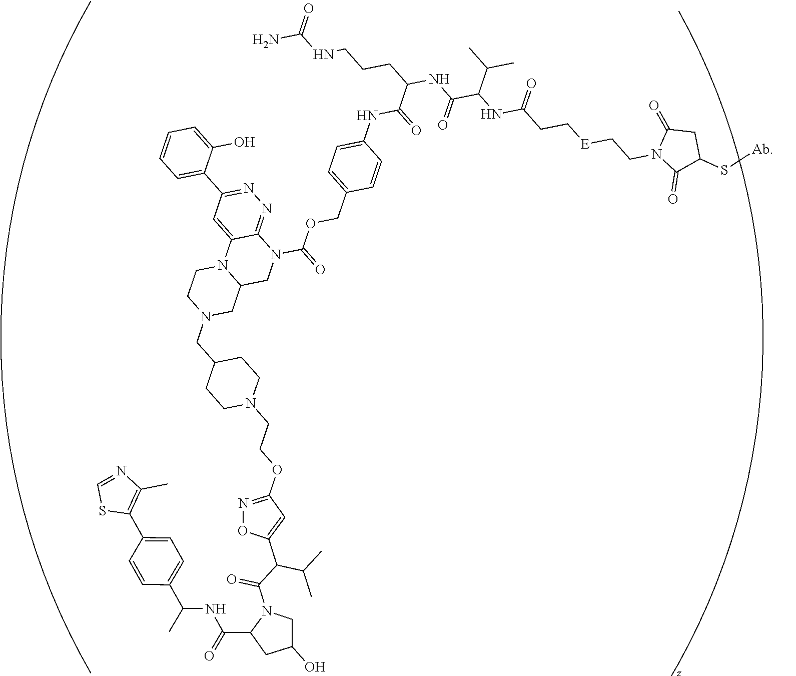

In some embodiments, the Antibody Drug Conjugate compound is represented by the structure of

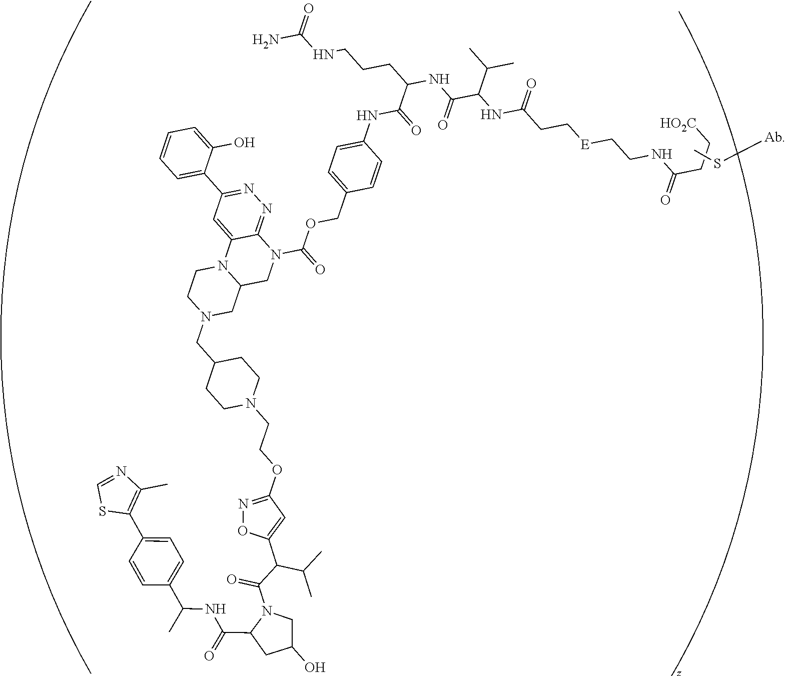

In some embodiments, the Antibody Drug Conjugate compound is represented by the structure of

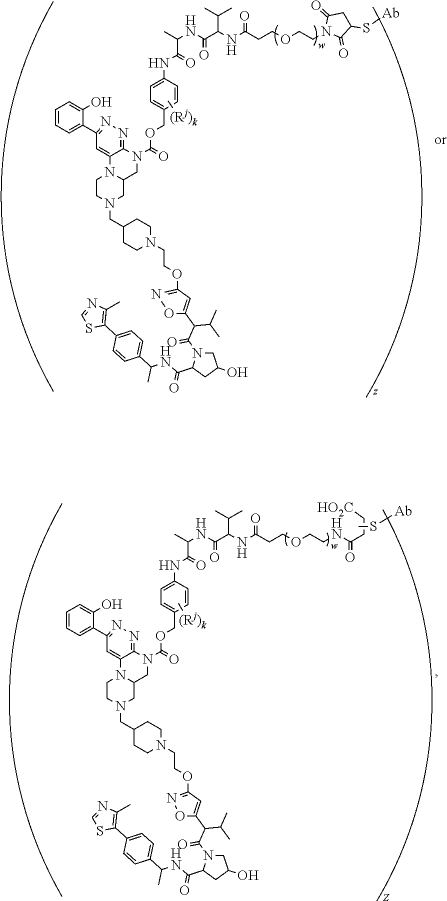

wherein subscript w is 8; R³ is methyl, —F, —Cl, or —C(O)NHCH₃; and subscript k is an 0 or 1.

In some embodiments, the Antibody Drug Conjugate compound is represented by the structure of

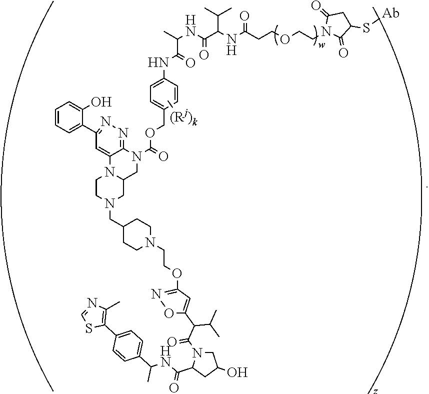

In some embodiments, the Antibody Drug Conjugate compound is represented by the structure of

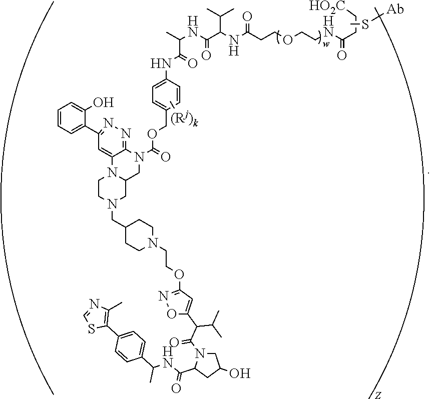

In some embodiments, the Antibody Drug Conjugate compound is represented by the structure of

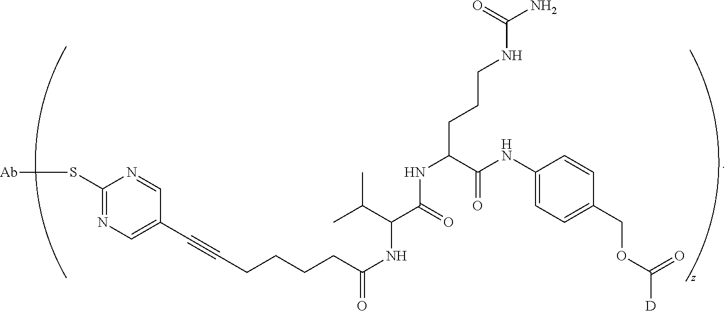

In some embodiments, the Antibody Drug Conjugate compound is represented by the structure of

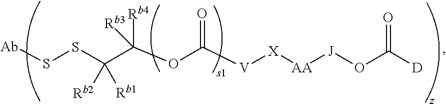

wherein R^{b1} and R^{b2} are each independently hydrogen, C₁-C₆ alkyl, or both R^{b1} and R^{b2}, together with the carbon to which they are attached, comprise a C₄-C₆ cycloalkyl or a C₄-C₆ heterocycloalkyl; R^{b3} and R^{b4}are each independently hydrogen or C₁-C₆ alkyl; subscript s1 is 0 or 1; and U, X, and AA are each absent. In some embodiments, U, X, AA, and J are each absent. In some embodiments, R^{b1} and R^{b2} are both hydrogen. In some embodiments, both R^{b1} and R^{b2}, together with the carbon to which they are attached, comprise a C₄-C₆ cycloalkyl or a C₄-C₆ heterocycloalkyl. In some embodiments, R^{b3} and R^{b4} are both hydrogen.

In some embodiments, the Antibody Drug Conjugate compound is represented by the structure of

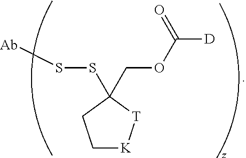

wherein T is absent or is —CH₂CH₂—; K is —CH₂— or —NH₂—. In some embodiments, T is absent. In some embodiments, T is —CH₂CH₂—. In some embodiments, K is —CH₂—. In some embodiments, K is —NH₂—.

In some embodiments, the Antibody Drug Conjugate compound is represented by the structure of

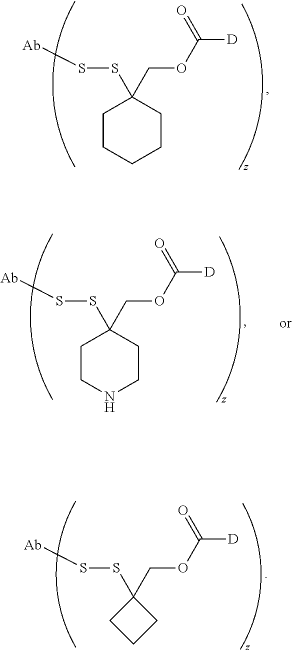

In some embodiments, the Antibody Drug Conjugate compound is represented by the structure of

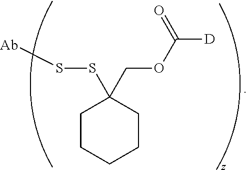

In some embodiments, the Antibody Drug Conjugate compound is represented by the structure of

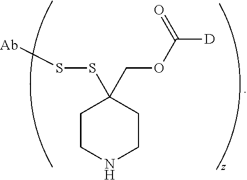

In some embodiments, the Antibody Drug Conjugate compound is represented by the structure of

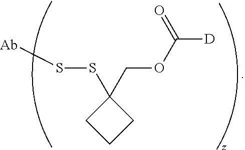

In some embodiments, the Antibody Drug Conjugate compound is represented by the structure of

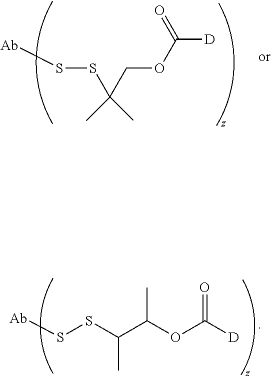

In some embodiments, the Antibody Drug Conjugate compound is represented by the structure of

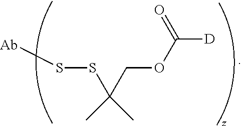

In some embodiments, the Antibody Drug Conjugate compound is represented by the structure of

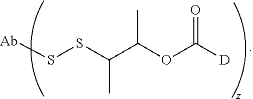

In some embodiments, the Antibody Drug Conjugate compound is represented by the structure of:

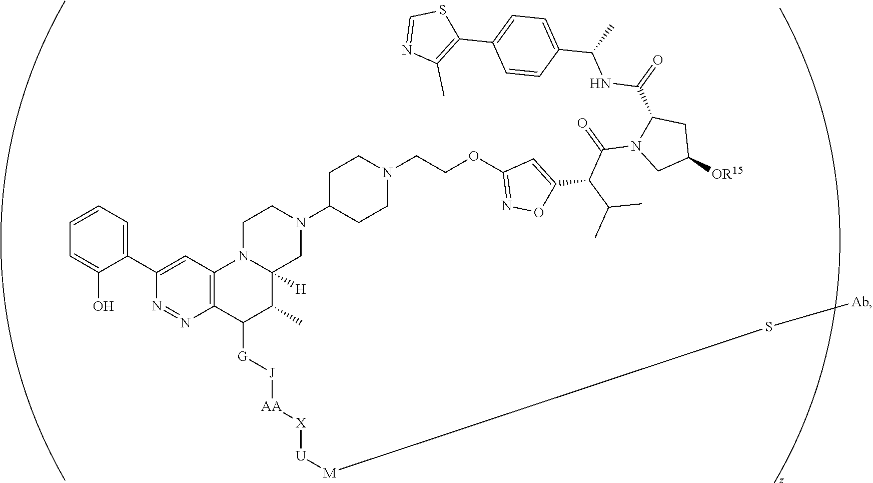

wherein S is a sulfur atom of the Ab and R¹⁵ is hydrogen or —PO₃H₂. In some embodiments, the Antibody Drug Conjugate compound is represented by the structure of:

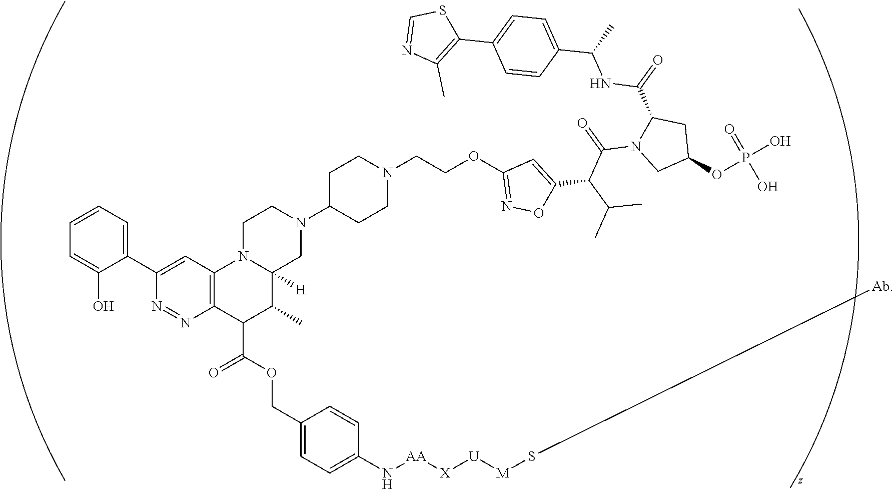

In some embodiments, the Antibody Drug Conjugate compound is represented by the structure of:

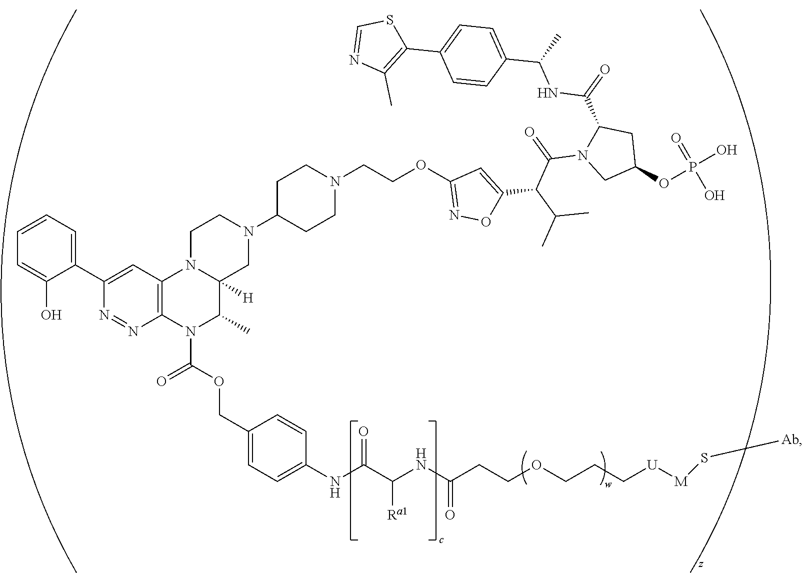

wherein S is a sulfur of the Ab; subscript c is an integer ranging between 1 and 4; subscript w is an integer ranging between 1 and 8; and U is absent or is —CH₂—(C═O)—(NH)—. In some embodiments, each R^{a1} is an independently selected side chain of alanine, valine, or citrulline. In some embodiments, subscript c is 2. In some embodiments, subscript w is 1. In other embodiments, subscript w is 8. In some embodiments, U is absent. In other embodiments, U is —CH₂—(C═O)—(NH)—. In some embodiments, M is

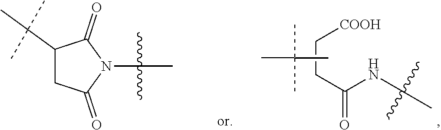

wherein the dashed line indicates the point of covalent attachment to the S of the Ab and the wavy line indicates the point of covalent attachment to U, when present, and to the remainder of the Antibody Drug conjugate structure when U is absent. In some embodiments, M is

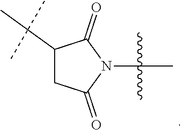

In some embodiments, M is

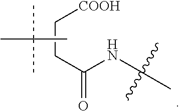

In some embodiments, the Antibody Drug Conjugate compound is represented by the structure of:

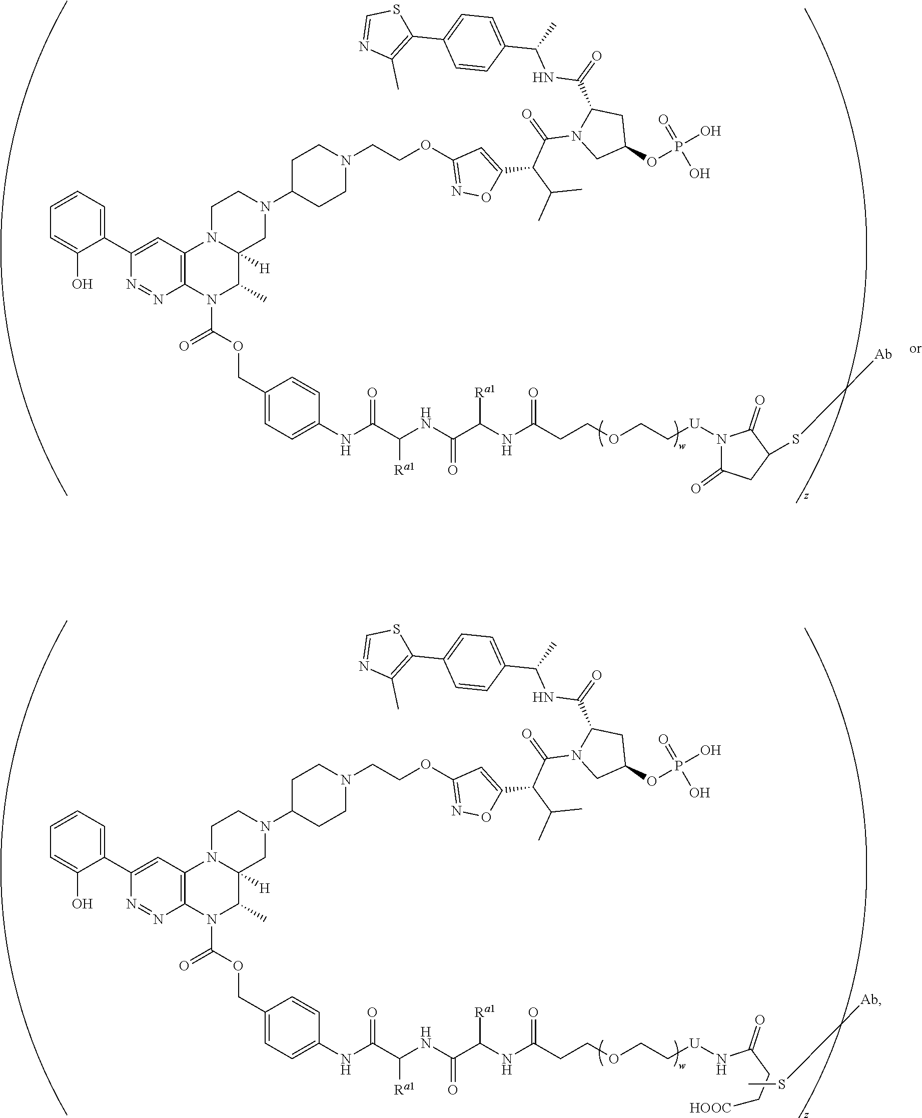

wherein S is a sulfur of the Ab; each R^{a1} is an independently selected side chain of alanine, valine, or citrulline; U is absent or is —CH₂—(C═O)—(NH)—; and subscript w is 1 or 8. In some embodiments, U is absent. In some embodiments, U is —CH₂—(C═O)—(NH)—. In some embodiments, each R^{a1} is independently selected from the side chain of valine, citrulline, and alanine.

In some embodiments, the Antibody Drug Conjugate compound is represented by the structure of:

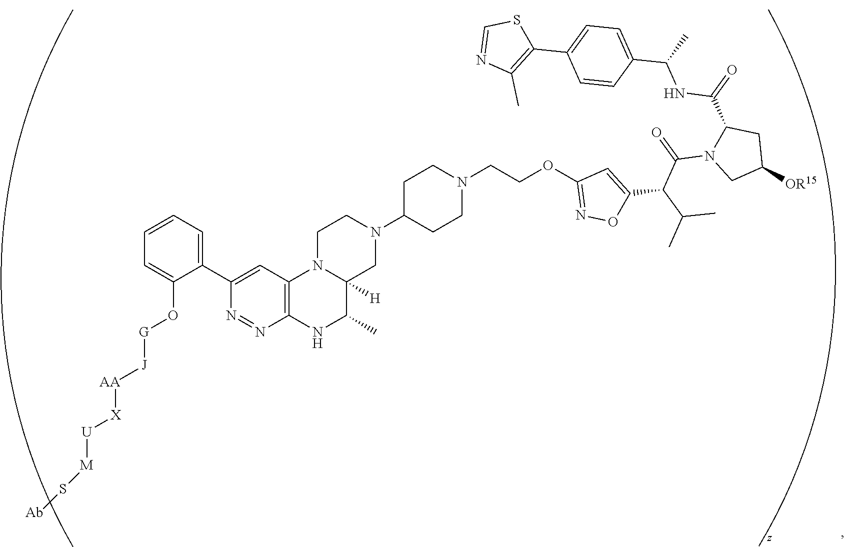

wherein S is a sulfur of the Ab and R¹⁵ is hydrogen or —PO₃H₂. In some embodiments, the Antibody Drug Conjugate compound is represented by the structure of:

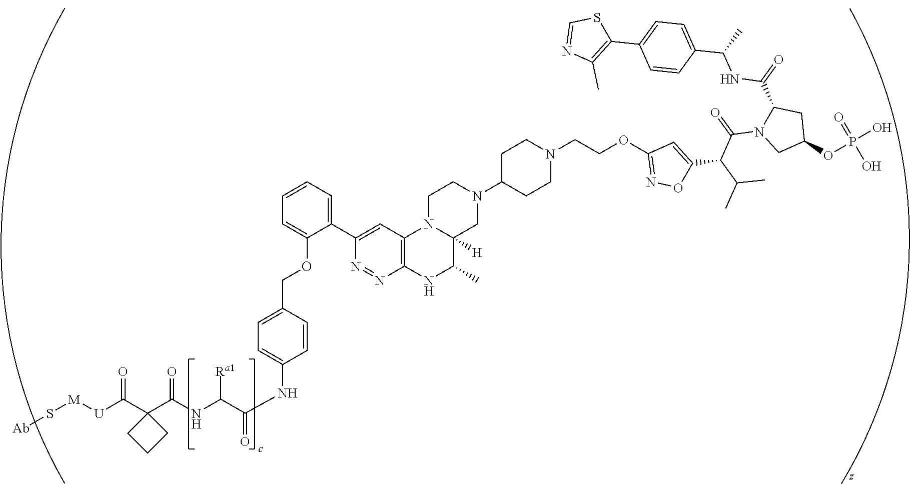

wherein is a sulfur of the Ab; subscript c is an integer ranging between 1 and 4; and U is —(CH₂CH₂O)_{w}CH₂CH₂(NH)—, wherein subscript w is an integer ranging between 1 and 4. In some embodiments, subscript c is 1 and R^{a1} is the side chain of citrulline. In some embodiments, U is —(CH₂CH₂O)_{w}CH₂CH₂(NH)—and subscript w is 4. In some embodiments, M is

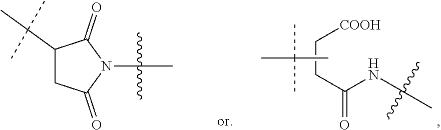

wherein the dashed line indicates the point of covalent attachment to the S of the Ab and the wavy line indicates the point of covalent attachment to U. In some embodiments, M is

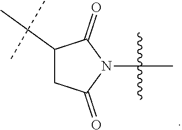

In some embodiments, M is

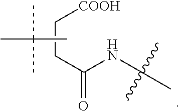

In some embodiments, the Antibody Drug conjugate compound is represented by the structure of:

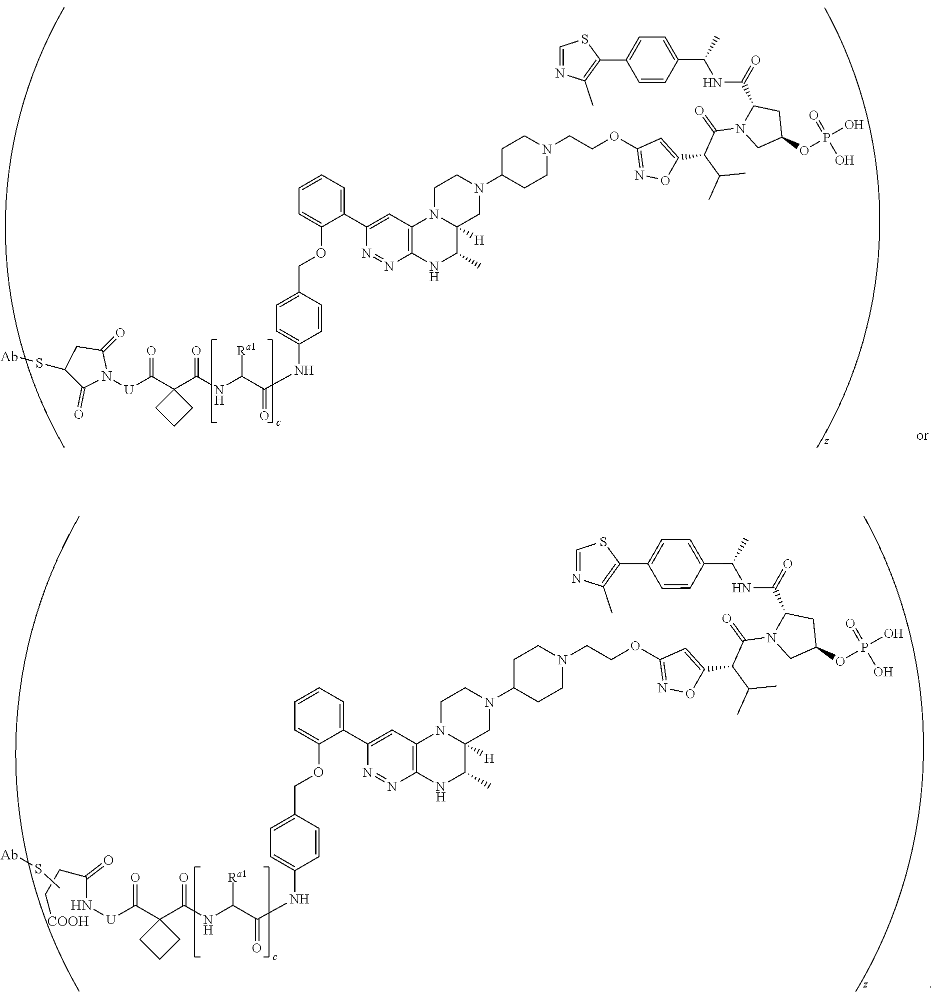

In some embodiments, the wavy line to M of Formula (II) indicates the point of covalent attachment to Ab and the wavy line to G of Formula (II) indicates the point of covalent attachment to D. In some embodiments, the wavy line to G of Formula (II) indicates the point of covalent attachment to a secondary alcohol group or a secondary amine group of D. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, the Antibody Drug Conjugate compound is represented by the structure of

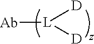

wherein,
Ab is an antibody or an antigen-binding fragment thereof;
each D is independently a degrader compound of Formula (I):

PTM-ULM (I)
wherein,
   PTM is a moiety of Formula IA:

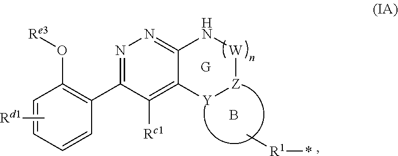

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (III):

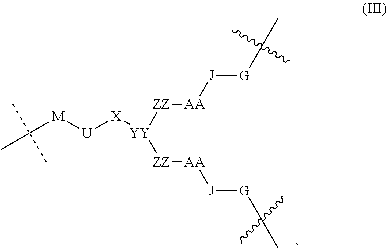

wherein,
   M is selected from the group consisting of:

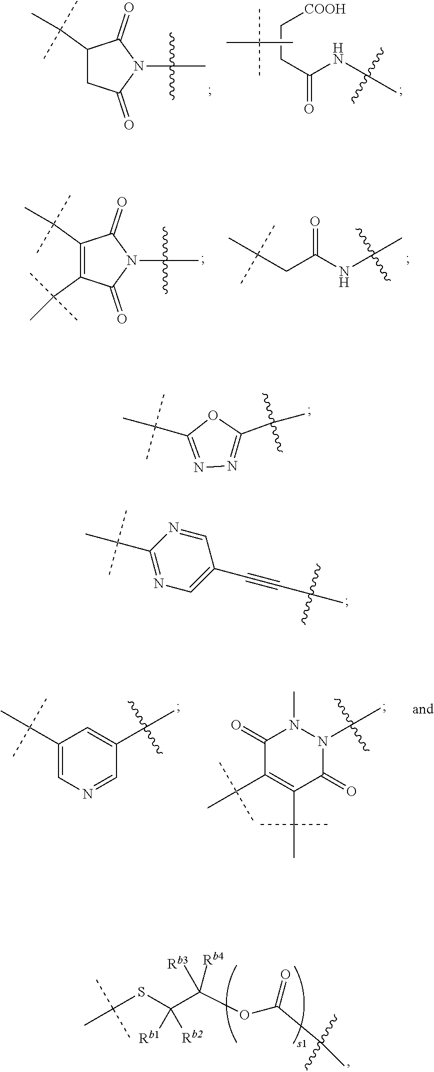

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl, subscript s1 is 0 or 1,
wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1}is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—OC(O)—, wherein R^{x5} is hydrogen or C₁-C₆ alkyl and R^{x6} is hydrogen, C₁-C₆ alkyl, or

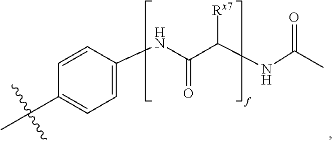

, wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of:

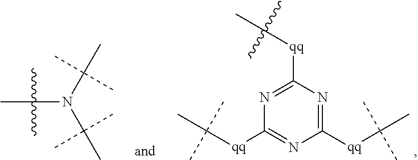

wherein
   each qq is independently —N(R^{y1})— or —O—, wherein R^{y1} is hydrogen or C₁-C₆ alkyl;
   the wavy line indicates the point of attachment to X, when present, or U, when X is absent, or M, when X and U are absent; and
   each dashed line indicates the point of attachment to ZZ when ZZ is present, or to AA when ZZ is absent, or to J when AA and ZZ are absent, or to G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of:

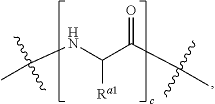

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

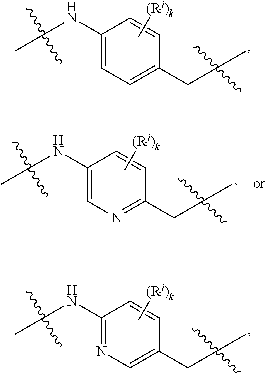

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
   each G is independently absent,

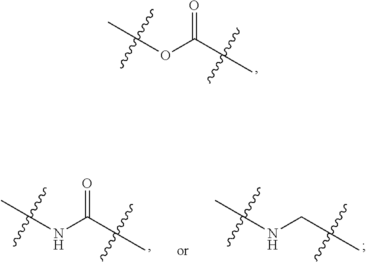

and
wherein the wavy line to M of Formula (III) indicates the point of covalent attachment to Ab and the wavy line to each G of Formula (III) indicates the point of covalent attachment to D,
wherein subscript z is an integer ranging from 1 to 14.

In some embodiments, L has the structure of Formula (III):

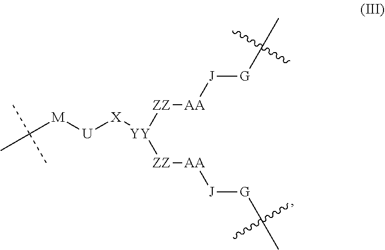

wherein YY is a branching unit.

In some embodiments, L has the structure of

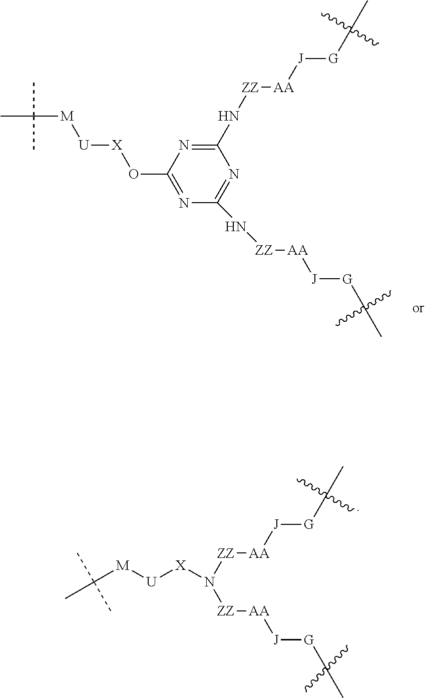

In some embodiments, L has the structure of

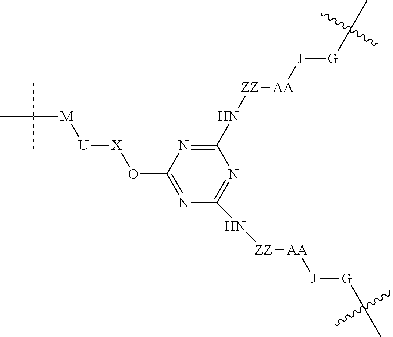

In some embodiments, L has the structure of

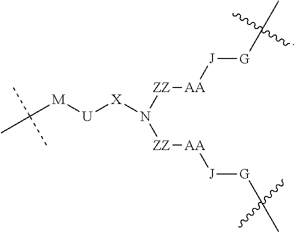

In some embodiments, L has the structure of

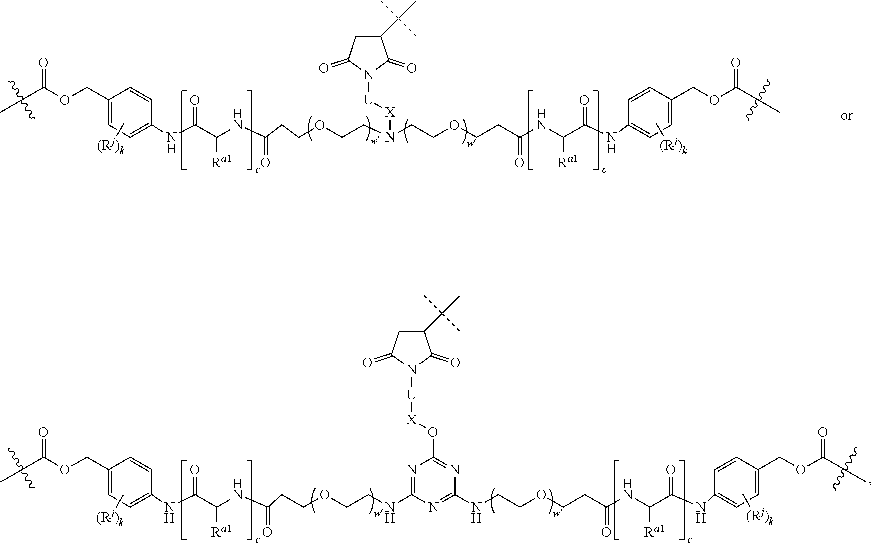

wherein
U is absent or is s(CH₂)_{b}(R^{v1})ₛₛ(C═O)ᵤ(NH)ᵥ—, wherein subscript b is 0, 1, 2, 3 4, or 5; subscript ss is 0 or 1; subscript u is 0 or 1; subscript v is 0 or 1; R^{v1} is —C₃-C₆ cycloalkyl- or —C₃-C₆ cycloakyl-C(O)NHCH₂R^{v2}, wherein R^{v2} is C₃-C₆ cycloalkyl, C₃-C₆ heterocycle, or C₃-C₆ heteroaryl; or —(CH₂CH₂—)CH₂CH₂(N)—, wherein subscript w is an integer ranging from 1 to 16;
   X is absent or is —(CH₂CH₂O)_{w}CH₂CH₂—(C═O)_{d}—, wherein subscript w is an integer ranging from 0 to 16 and subscript d is 0 or 1;
   subscript c is an integer ranging from 1 to 12 and each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine;
   each R^{j} is independently fluorine, chlorine, methyl, or —C(O)NHCH₃; and
   subscript k is an integer ranging from 0 to 4.

In some embodiments, L has the structure of

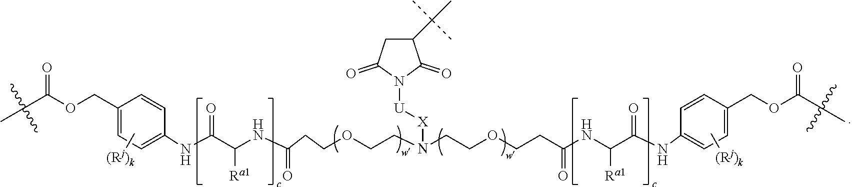

In some embodiments, L has the structure of

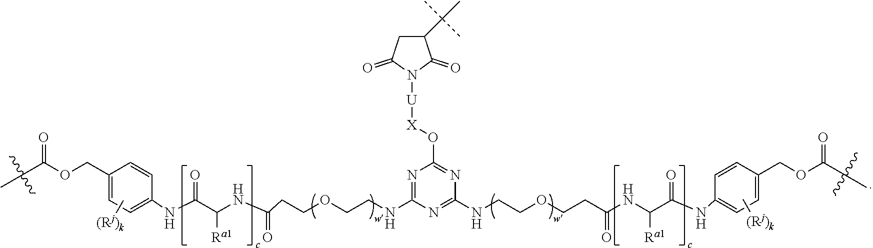

In some embodiments, the wavy line to M of Formula (III) indicates the point of covalent attachment to Ab and the wavy line to each G of Formula (III) indicates the point of covalent attachment to D. In some embodiments, the wavy line to each G of Formula (III) indicates the point of covalent attachment to a secondary alcohol group or a secondary amine group of D. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, the Antibody Drug Conjugate compound is represented by the structure of

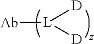

wherein,
Ab is an antibody or an antigen-binding fragment thereof;
each D is independently a degrader compound of Formula (I):

PTM-ULM (I),
wherein,
   PTM is a moiety of Formula IA:

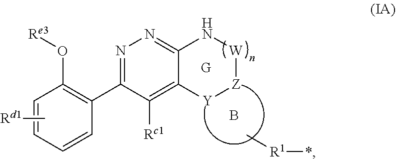

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (IIb):

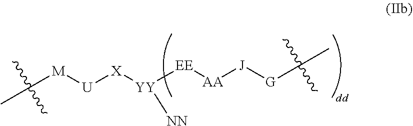

wherein,
   subscript dd is 2;
   M is selected from the group consisting of:

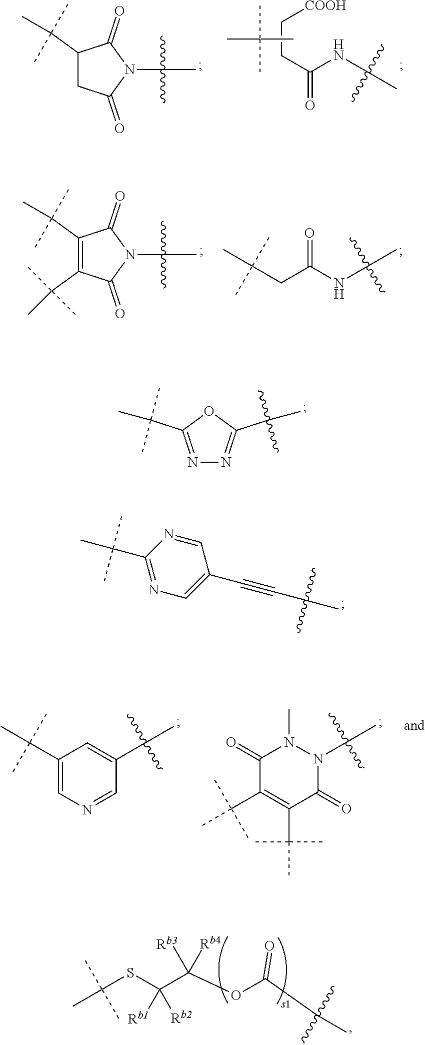

wherein
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl, subscript s1 is 0 or 1,
wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—, wherein R^{x5} is hydrogen or C₁-C₆ alkyl and R^{x6} is hydrogen, C₁-C₆ alkyl, or

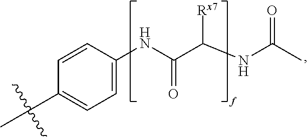

wherein subscript f is an integer ranging from 0 to 4 and each R^{x7} is independently hydrogen, —COOH, —NH₂, C₁-C₆ alkyl, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit having the structure of:

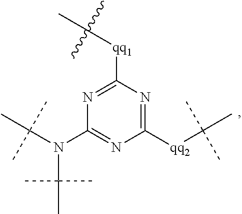

wherein
   qq₁ and qq₂ are each independently —N(R^{y1})— or —O—, wherein R^{y1} is hydrogen or C₁-C₆ alkyl, and
   the wavy line of YY indicates the point of attachment to X, when present, or to U when X is absent, or to M when X and U are absent; and
   the dashed lines of YY indicate the point of attachment to EE when EE is present, or to AA when EE is absent, or to J when AA and EE are absent, or to G when AA, EE, and J are absent, and to NN;
NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
each EE is independently absent or —(CH₂CH₂O)_{x′}CH₂CH₂C(O)—, wherein subscript x′ is an integer ranging from 0 to 16;
each AA is independently absent or has the structure of:

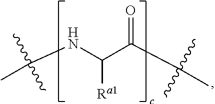

wherein subscript c is an integer ranging from 1 to 12; each R^{a1} is independently —COOH, —NH₂, or an independently selected side chain of an amino acid; each J is independently absent, —(R^{j3})N(C(R^{j1})(R^{j2}))ₘ₁—, or has the structure of:

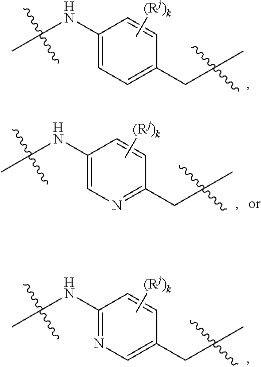

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
each G is independently absent,

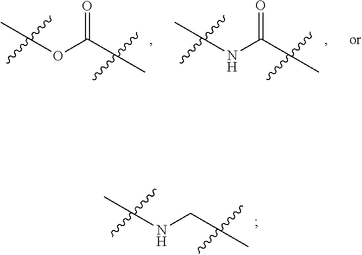

and
wherein the wavy line to M of Formula (IIb) indicates the point of covalent attachment to Ab and the wavy line to G of Formula (IIb) indicates the point of covalent attachment to an independently selected D.

In some embodiments, L is a linker of Formula (IIb) and has the structure of:

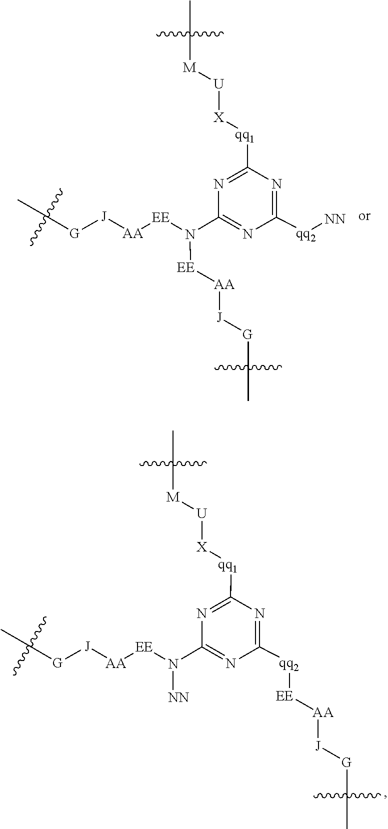

wherein qq₁ and qq₂ are each independently —N(R^{y1})— or —O—, wherein R^{y1} is hydrogen or C₁-C₆ alkyl.

In some embodiments, the wavy line to M of Formula (IIb) indicates the point of covalent attachment to Ab and the wavy line to each G of Formula (IIb) indicates the point of covalent attachment to D. In some embodiments, the wavy line to each G of Formula (IIb) indicates the point of covalent attachment to a secondary alcohol group or a secondary amine group of D. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, L is a Linker of Formula (III) and is covalently attached to two degrader compounds (D) of Formula (I). In some embodiments, L is a Linker of Formula (III) and is covalently attached to two of the same degrader compounds of Formula (I).

In some embodiments, L is a Linker of Formula (III) and is covalently attached to two degrader compounds of Formula (I), and each degrader compound is a different degrader compound of Formula (I). For example, in some embodiments, L is a Linker of Formula (III) and is covalently attached to a degrader compound of Formula (I) wherein the ULM of Formula (I) binds Von Hippel-Lindau E3 Ubiquitin Ligase and to a degrader compound of Formula (I) wherein the ULM of Formula (I) binds Cereblon E3 Ubiquitin Ligase. In some such embodiments, the ULM of Formula (I) that binds Von Hippel-Lindau E3 Ubiquitin Ligase has the Formula ULM-I-VHL, as described herein, and the ULM of (I) that binds Cereblon E3 Ubiquitin Ligase has the formula of ULM-II-CRBN, as described herein.

In some embodiments, L is a Linker of Formula (III) and is covalently attached to two different degrader compounds of Formula (I) that both bind Von Hippel-Lindau E3 Ubiquitin Ligase. In some embodiments, L is covalently attached to two different degrader compounds of Formula (I) that both bind Cereblon E3 Ubiquitin Ligase.

In some embodiments, L is a Linker of Formula (IIb) and is covalently attached to two degrader compounds (D) of Formula (I). In some embodiments, L is a Linker of Formula (IIb) and is covalently attached to two of the same degrader compounds of Formula (I).

In some embodiments, L is a Linker of Formula (IIb) and is covalently attached to two degrader compounds of Formula (I), and each degrader compound is a different degrader compound of Formula (I). For example, in some embodiments, L is a Linker of Formula (IIb) and is covalently attached to a degrader compound of Formula (I) wherein the ULM of Formula (I) binds Von Hippel-Lindau E3 Ubiquitin Ligase and to a degrader compound of Formula (I) wherein the ULM of Formula (I) binds Cereblon E3 Ubiquitin Ligase. In some such embodiments, the ULM of Formula (I) that binds Von Hippel-Lindau E3 Ubiquitin Ligase has the Formula ULM-I-VHL, as described herein, and the ULM of (I) that binds Cereblon E3 Ubiquitin Ligase has the formula of ULM-II-CRBN, as described herein.

In some embodiments, L is a Linker of Formula (IIb) and is covalently attached to two different degrader compounds of Formula (I) that both bind Von Hippel-Lindau E3 Ubiquitin Ligase. In some embodiments, L is covalently attached to two different degrader compounds of Formula (I) that both bind Cereblon E3 Ubiquitin Ligase.

In some embodiments, z is an integer ranging from 2 to 8. In some embodiments, z is 4. In some embodiments, Ab binds to prostate-specific membrane antigen (PSMA). In some embodiments, Ab is an anti-PSMA antibody. In some embodiments, Ab binds to CD33 antigen. In some embodiments, Ab is an anti-CD33 antibody.

In some embodiments, provided is an Antibody Drug Conjugate compound of Table 1, or a pharmaceutically acceptable salt thereof.

TABLE 1
Antibody Drug Conjugate Compounds

| Com- | |
|---|---|
| pound | |
| No. | Compound Structure |
| 1.01 | 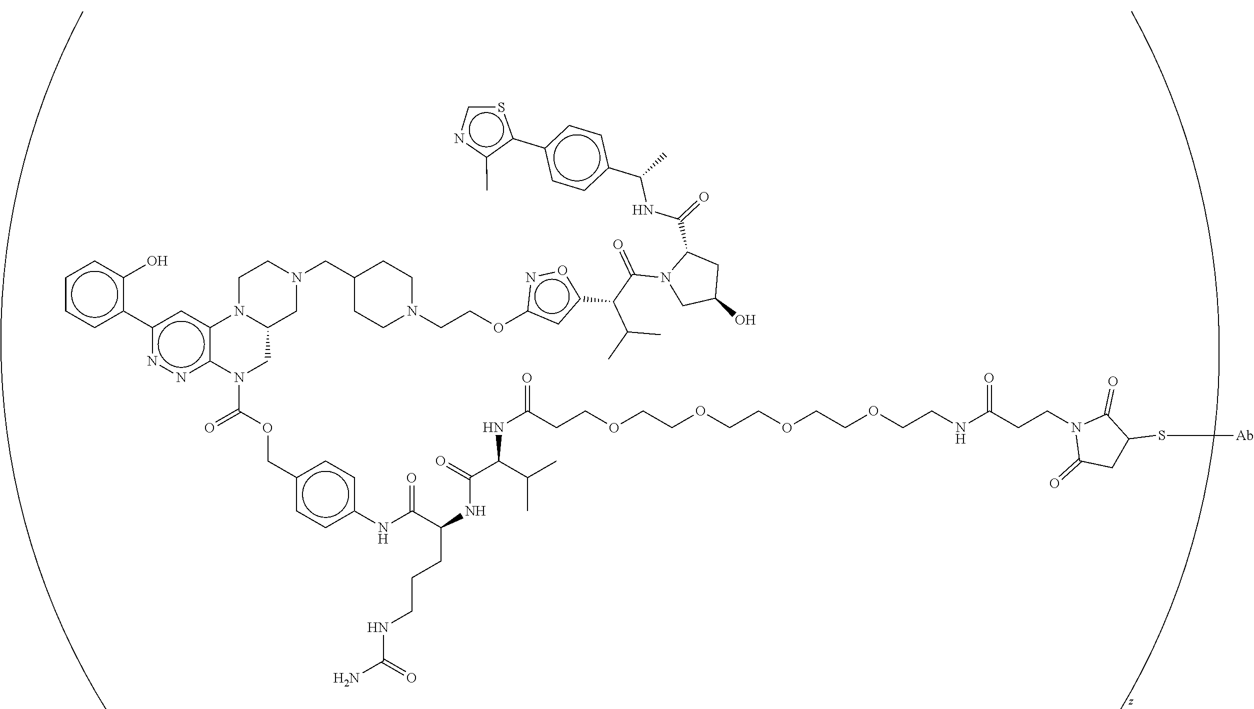 |
| 1.02 | 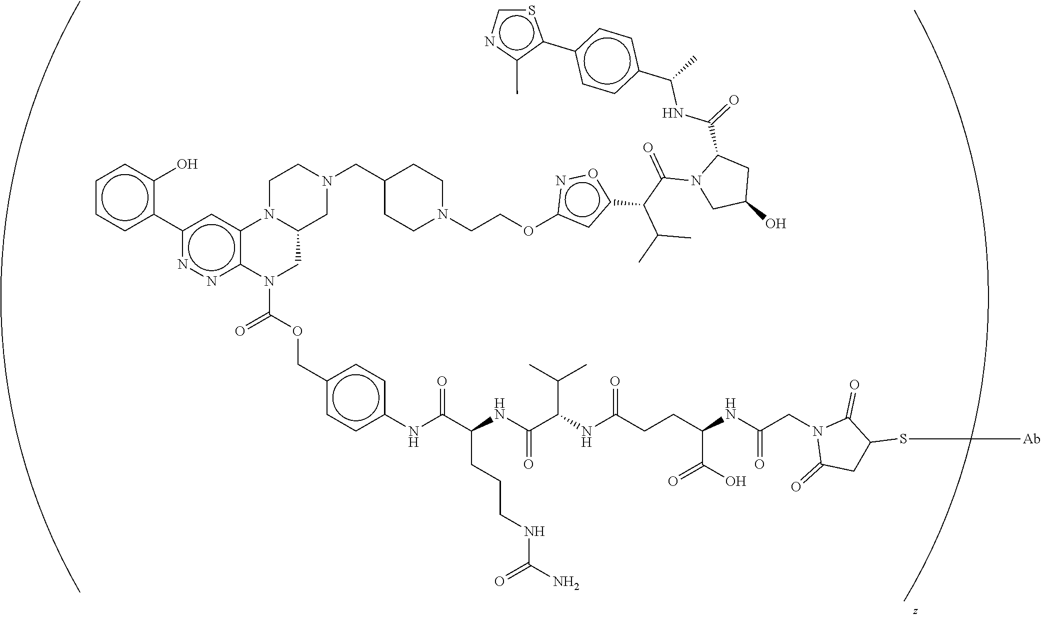 |
| 1.03 | 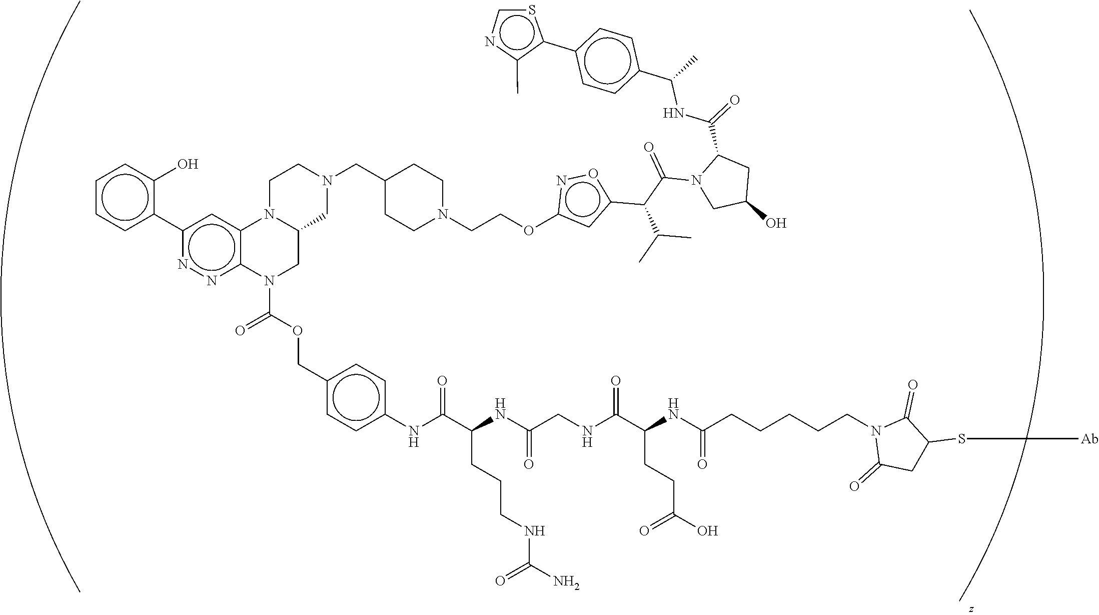 |
| 1.04 | 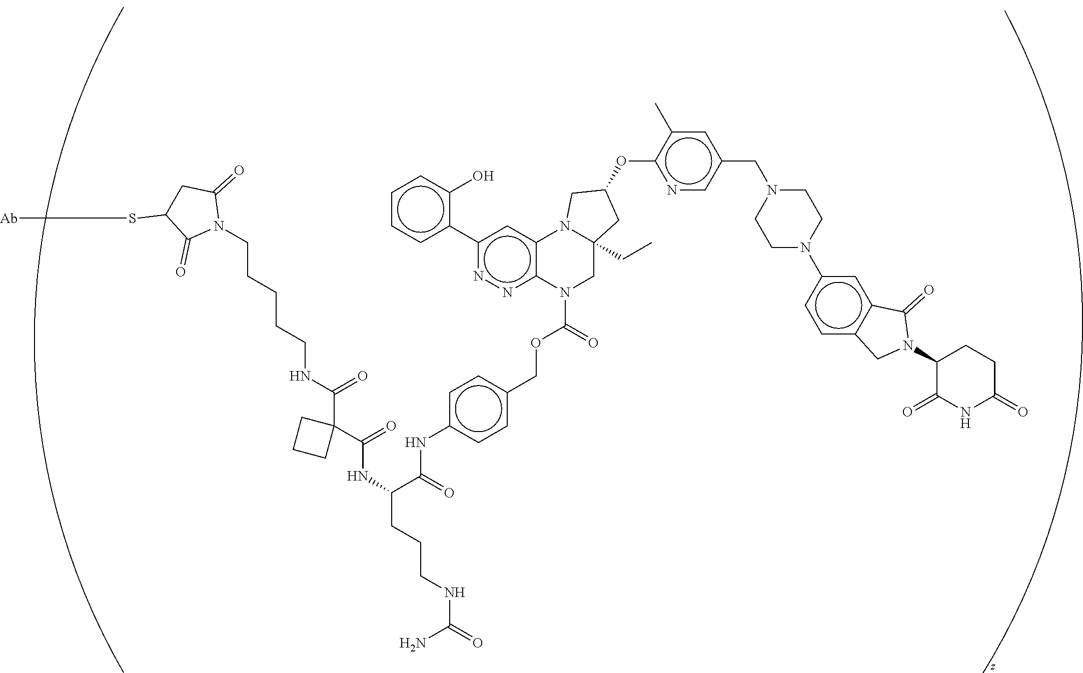 |
| 1.05 | 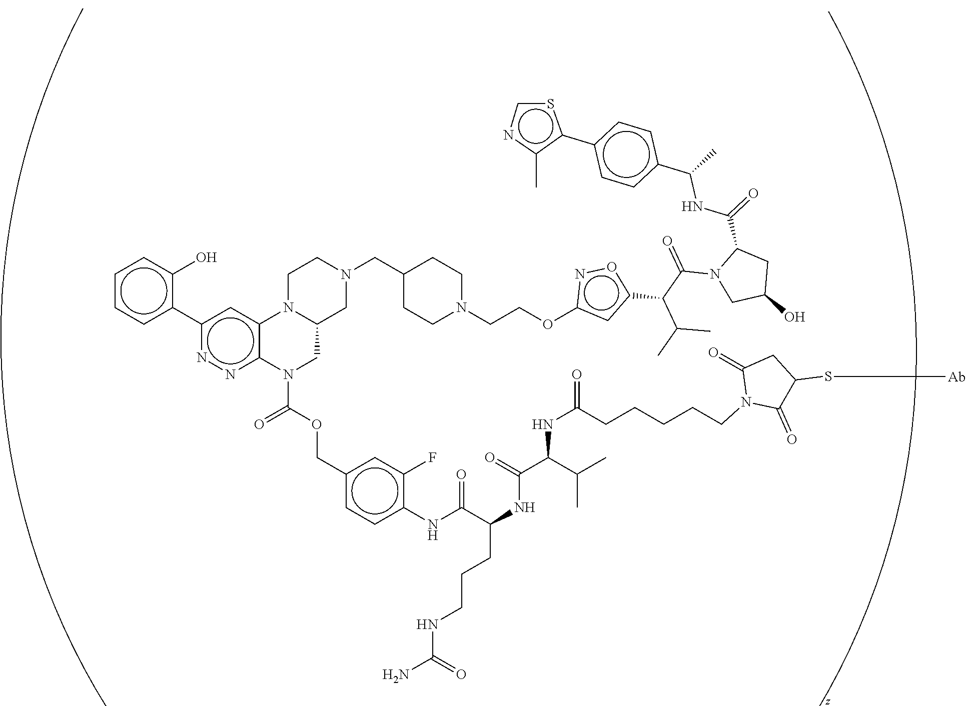 |
| 1.06 | 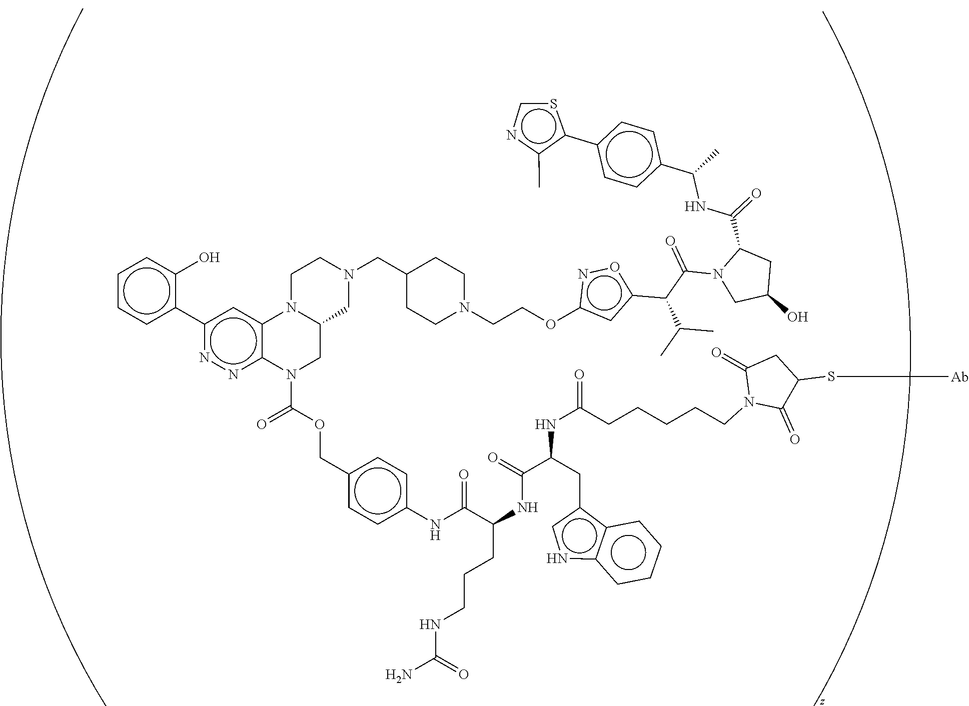 |
| 1.07 | 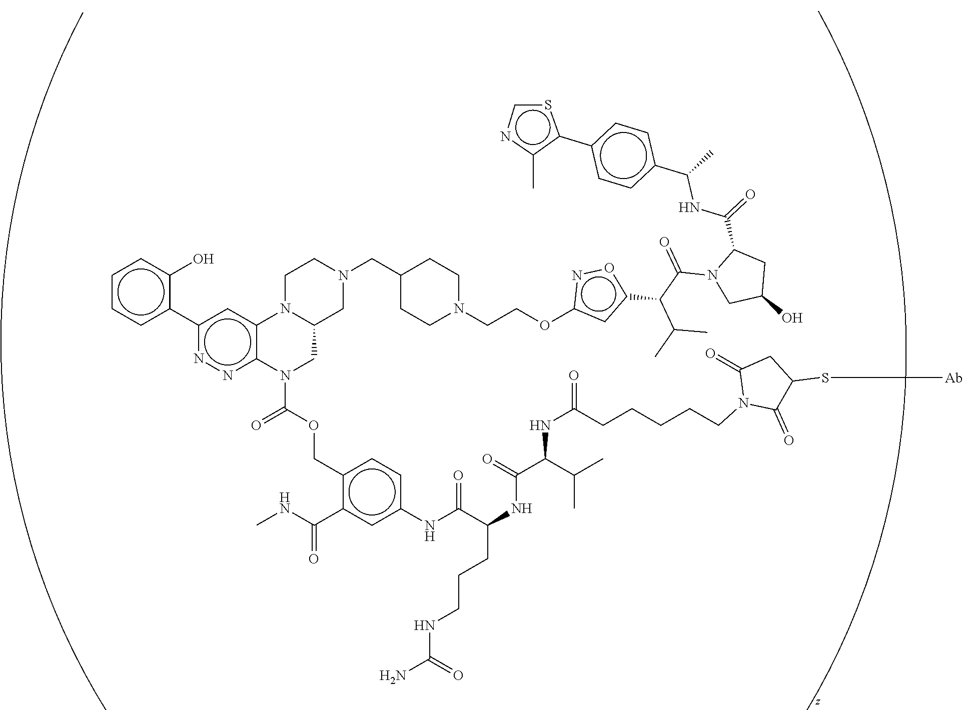 |
| 1.08 | 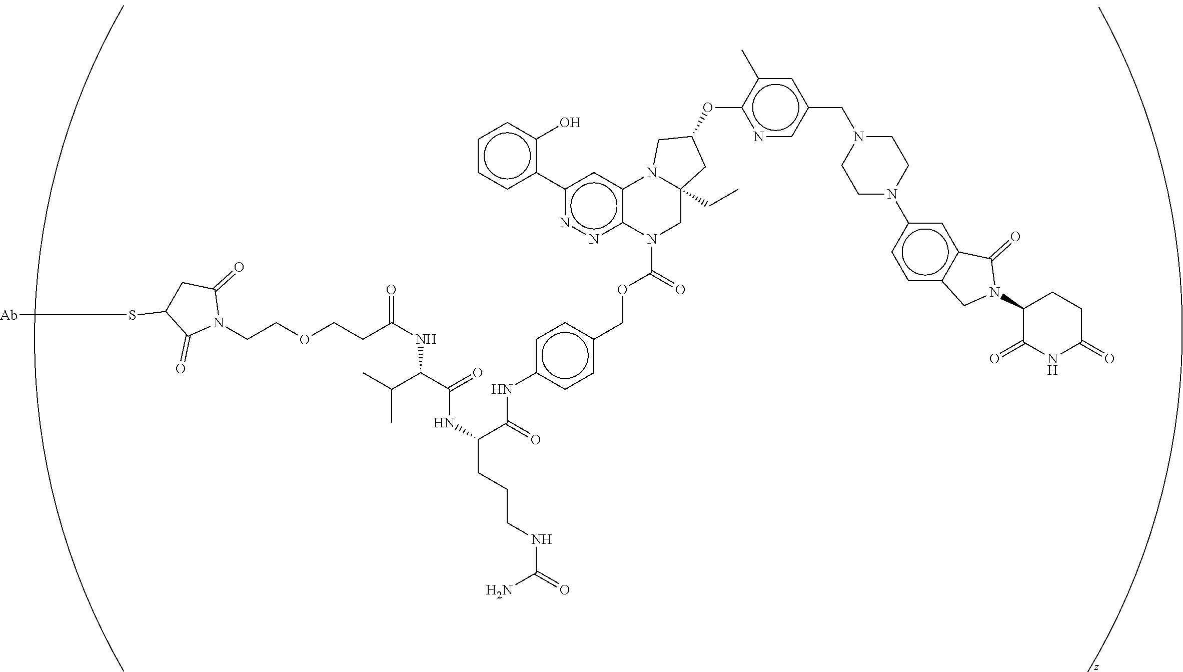 |
| 1.09 | 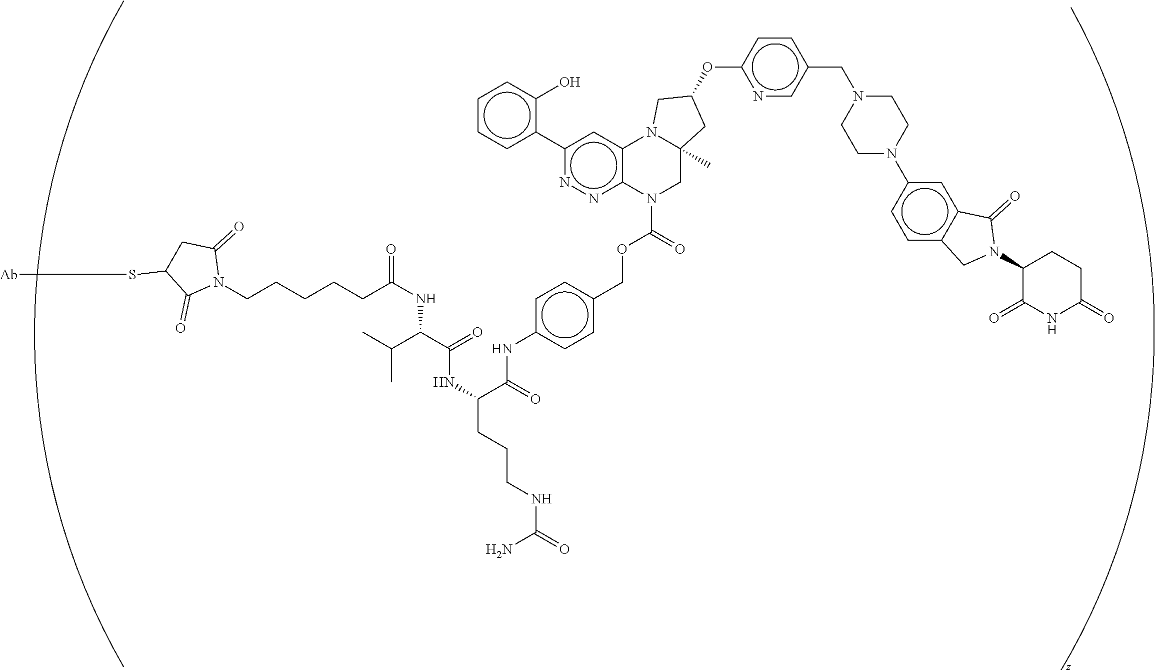 |
| 1.10 | 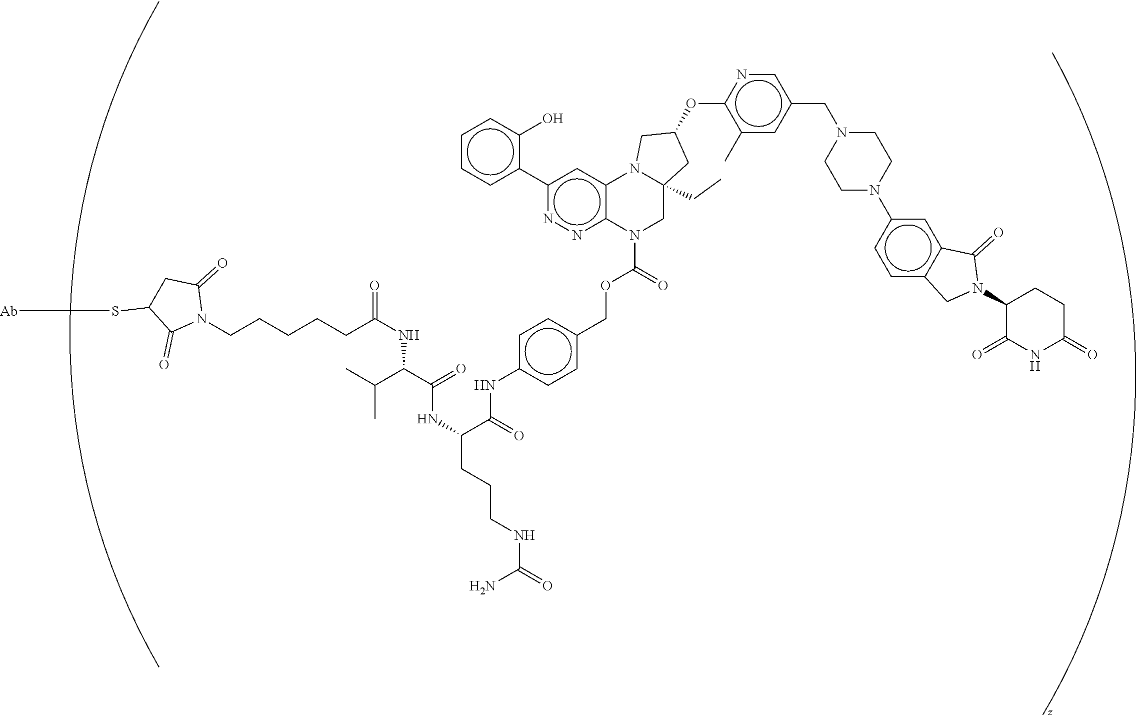 |
| 1.11 | 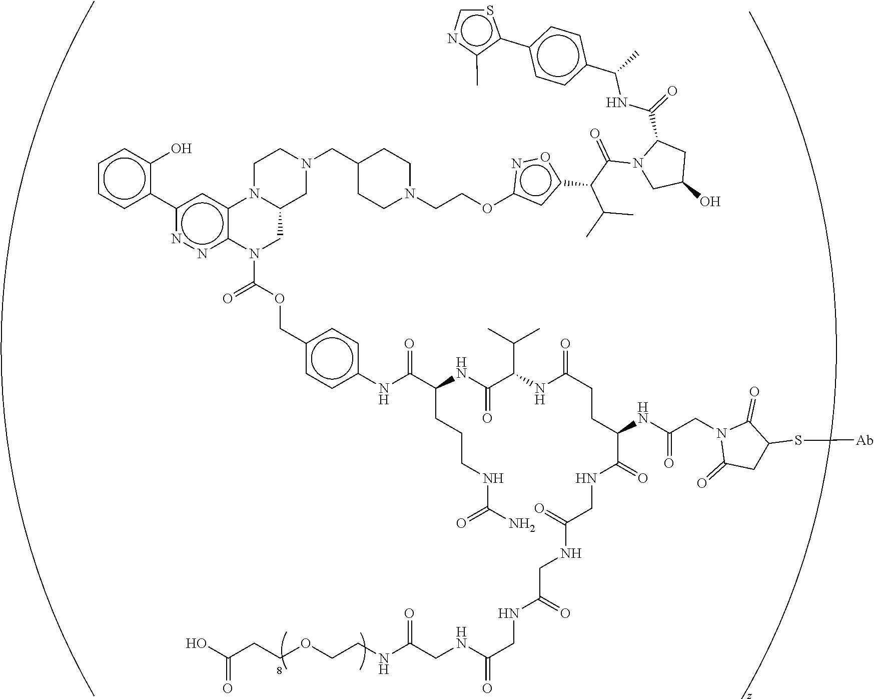 |
| 1.12 | 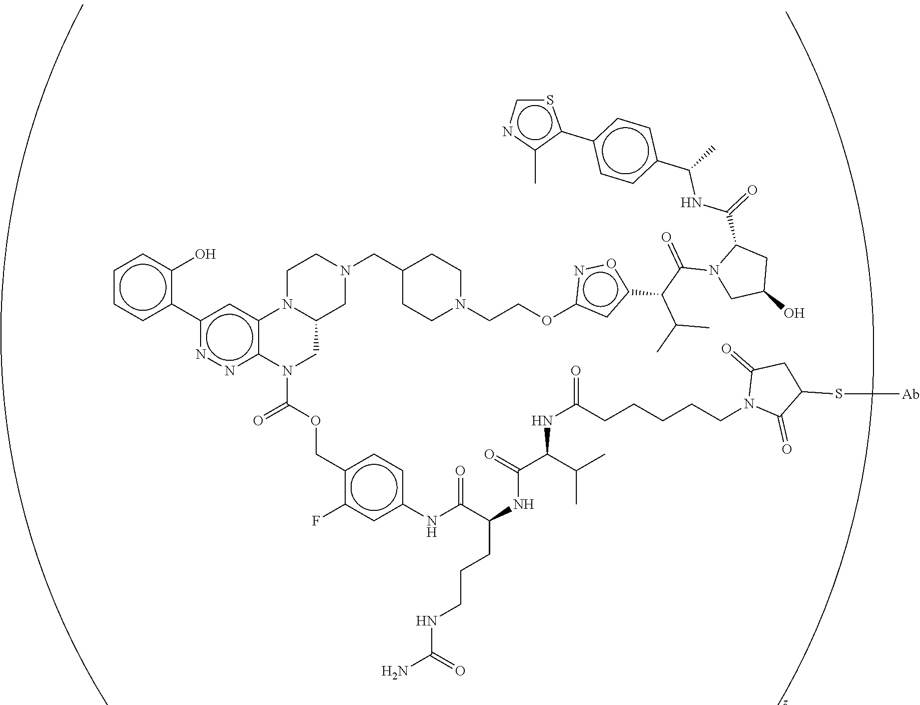 |
| 1.13 | 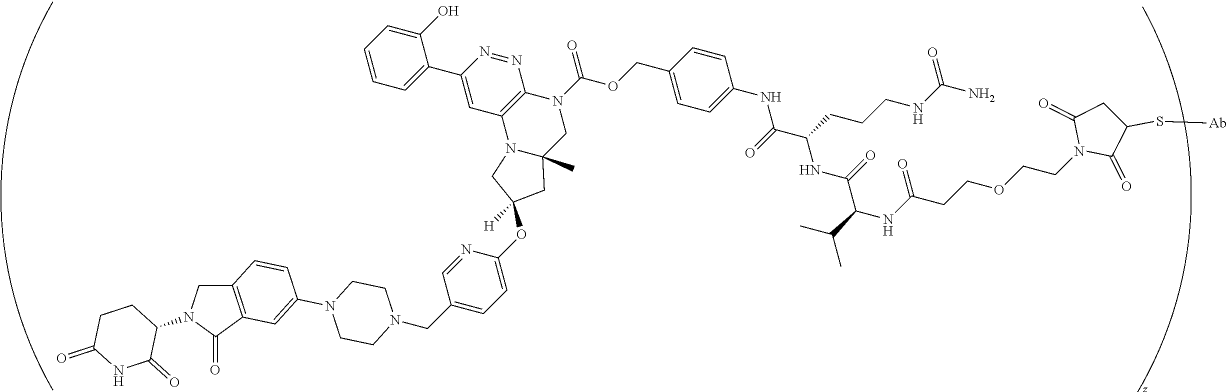 |
| 1.14 | 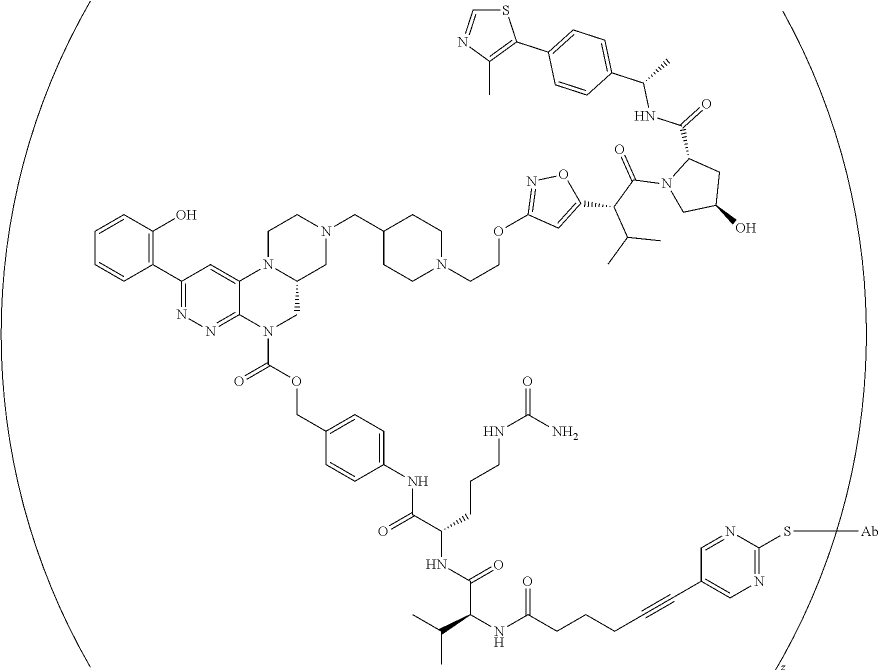 |
| 1.15 | 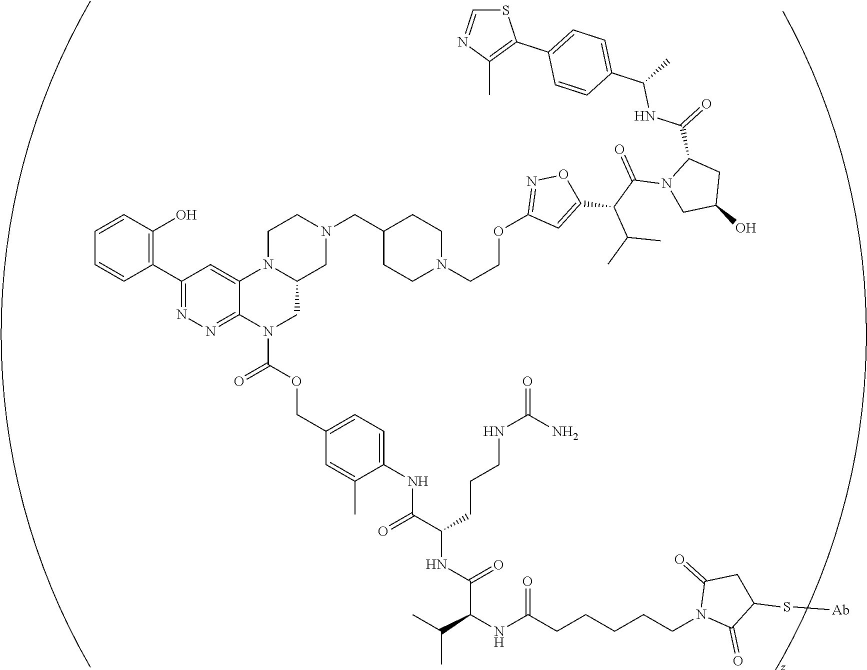 |
| 1.16 | 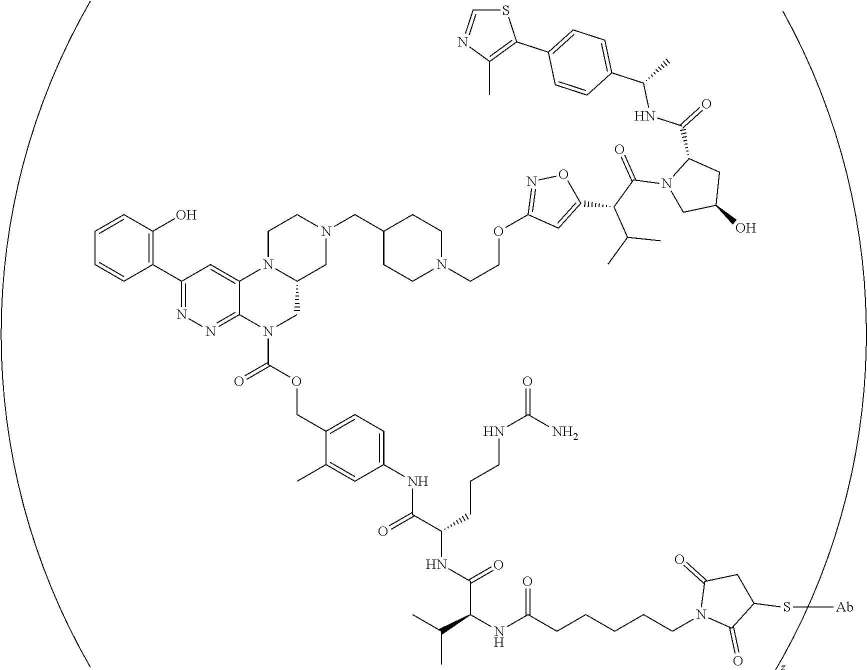 |
| 1.17 | 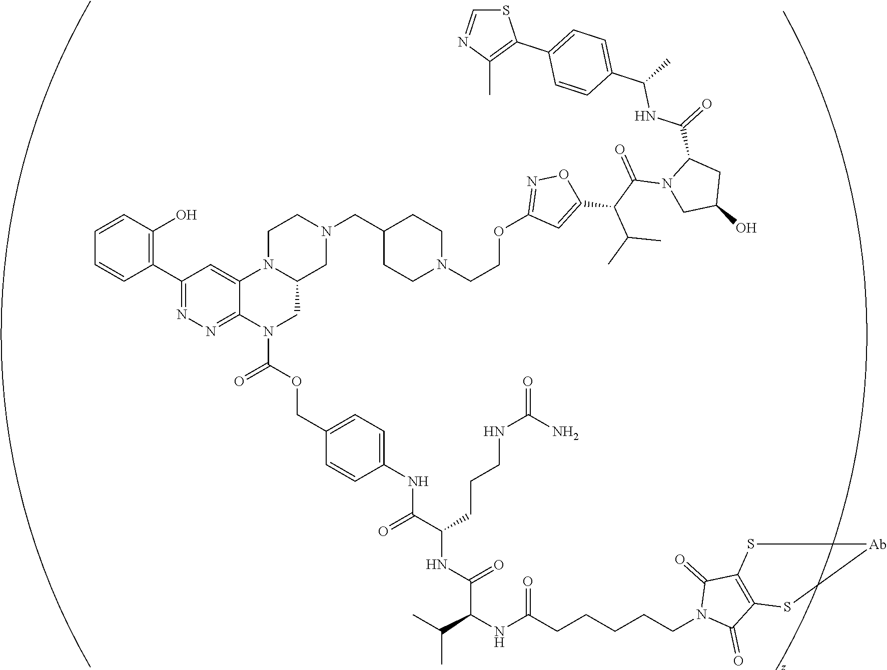 |
| 1.18 | 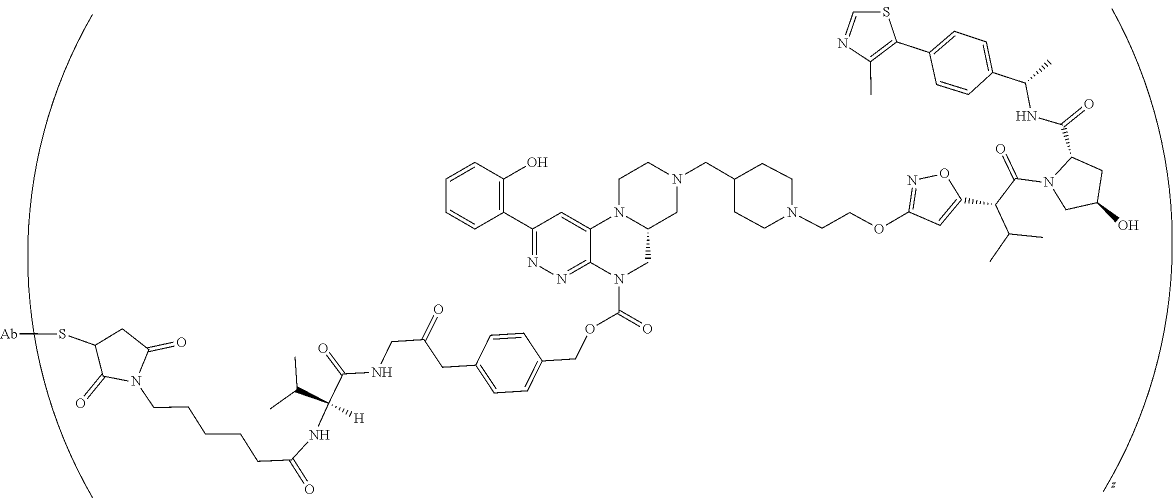 |
| 1.19 | 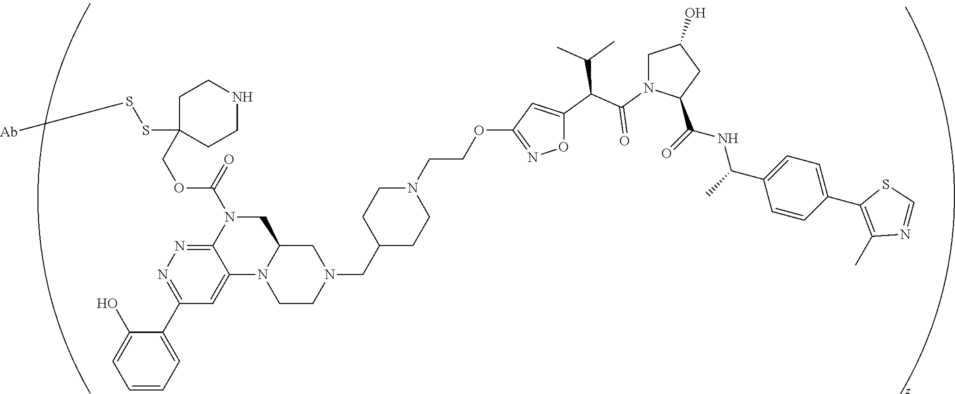 |
| 1.20 | 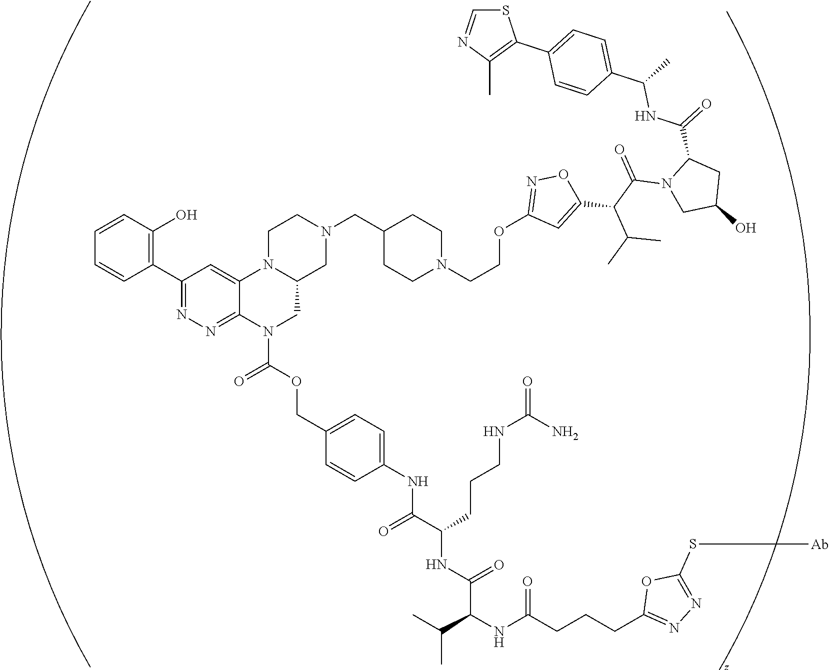 |
| 1.21 | 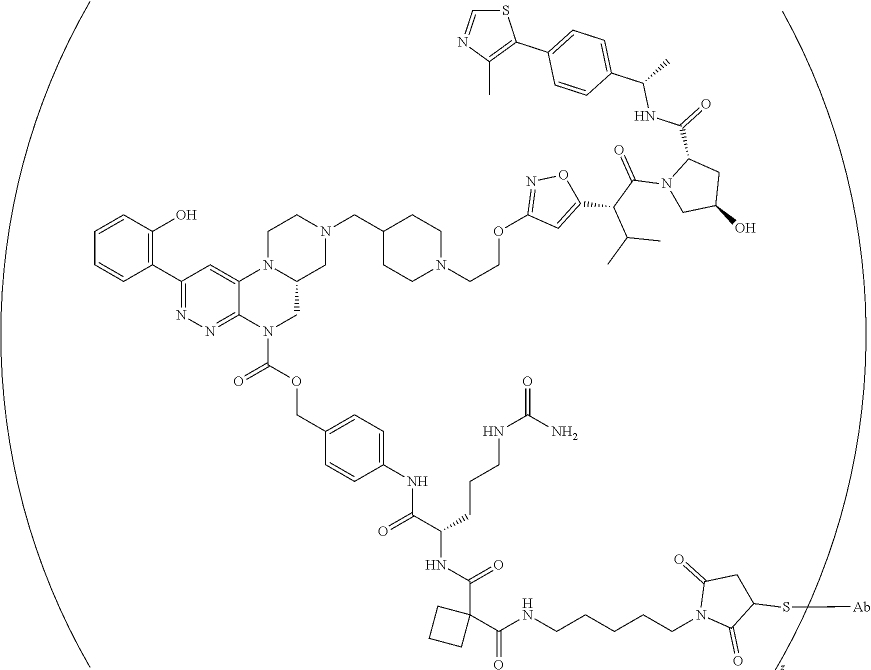 |
| 1.22 | 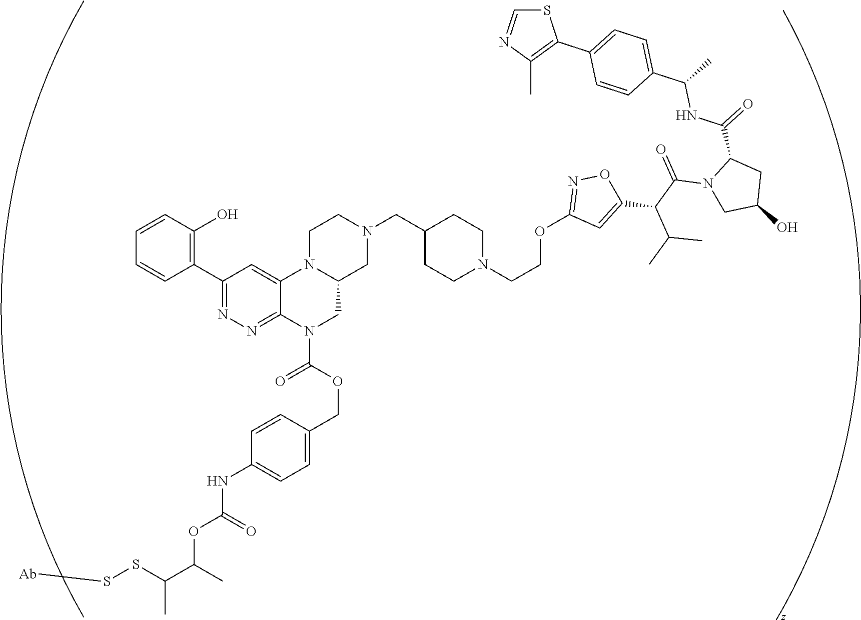 |
| 1.23 | 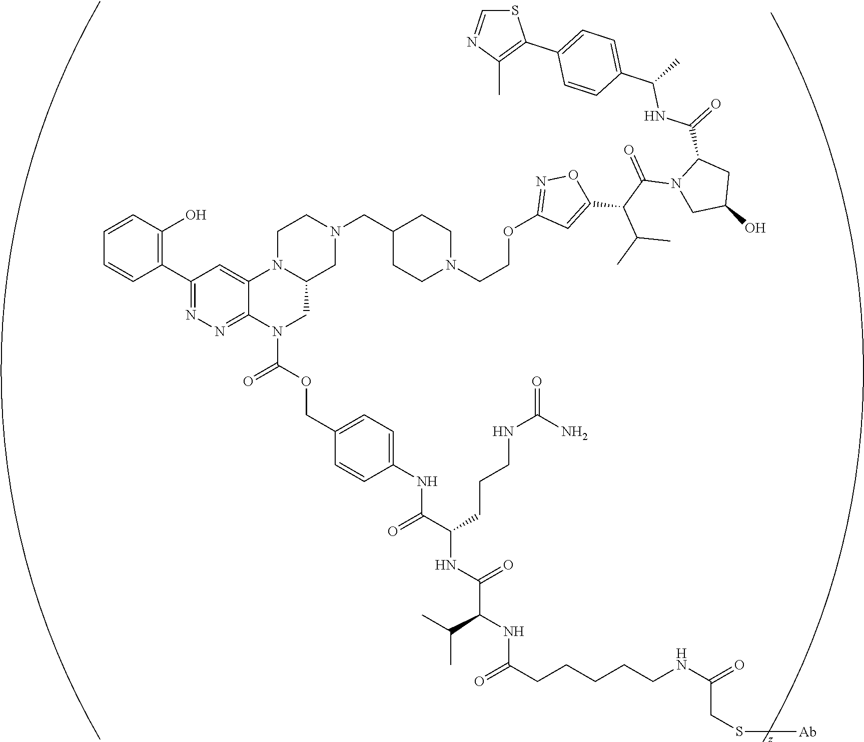 |
| 1.24 | 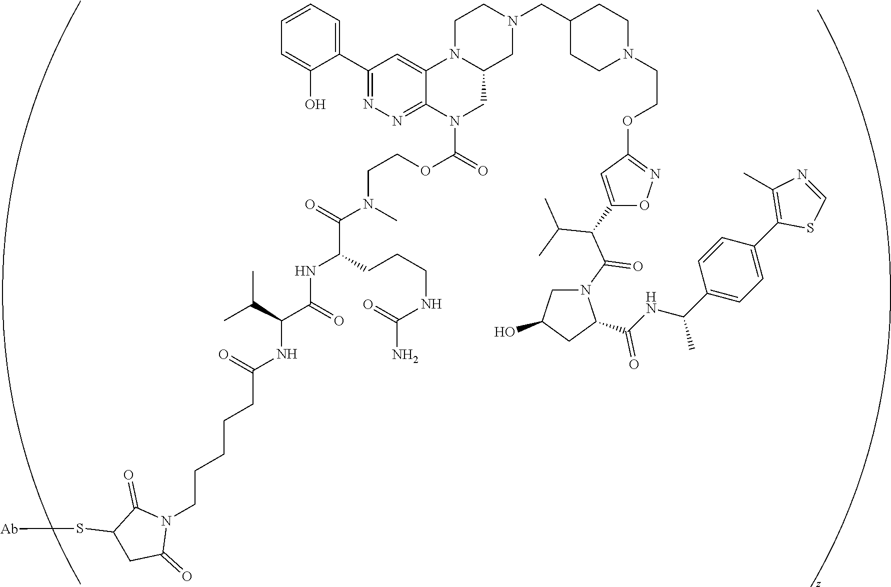 |
| 1.25 | 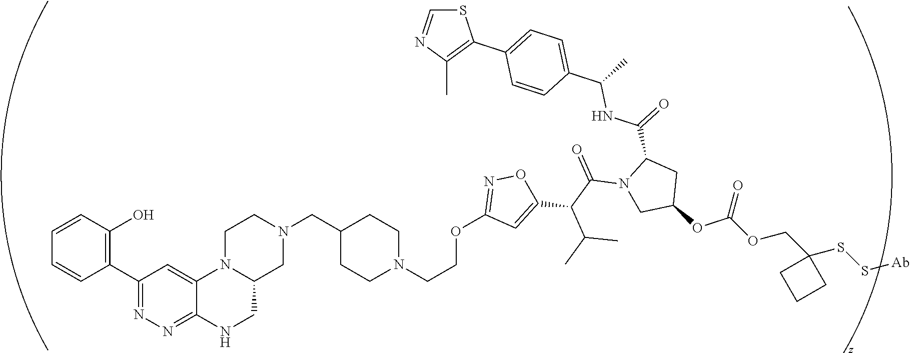 |
| 1.26 | 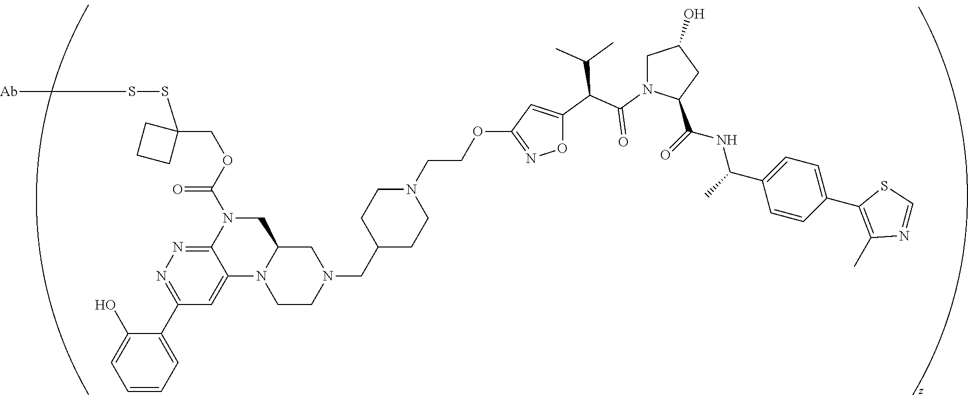 |
| 1.27 | 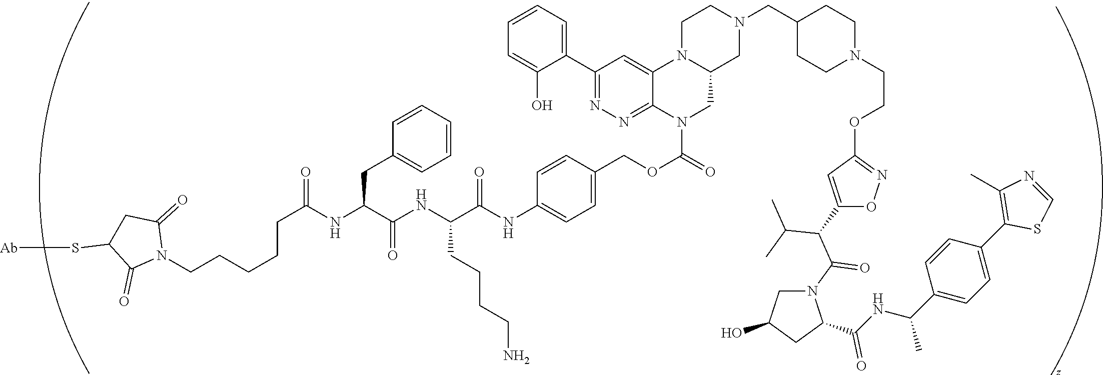 |
| 1.28 | 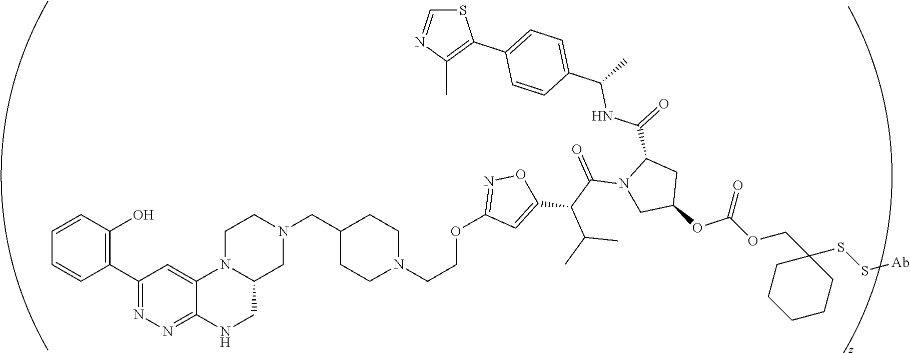 |
| 1.29 | 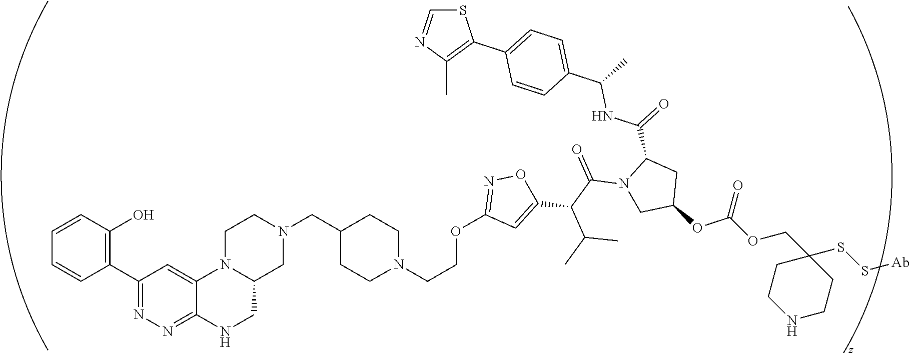 |
| 1.30 | 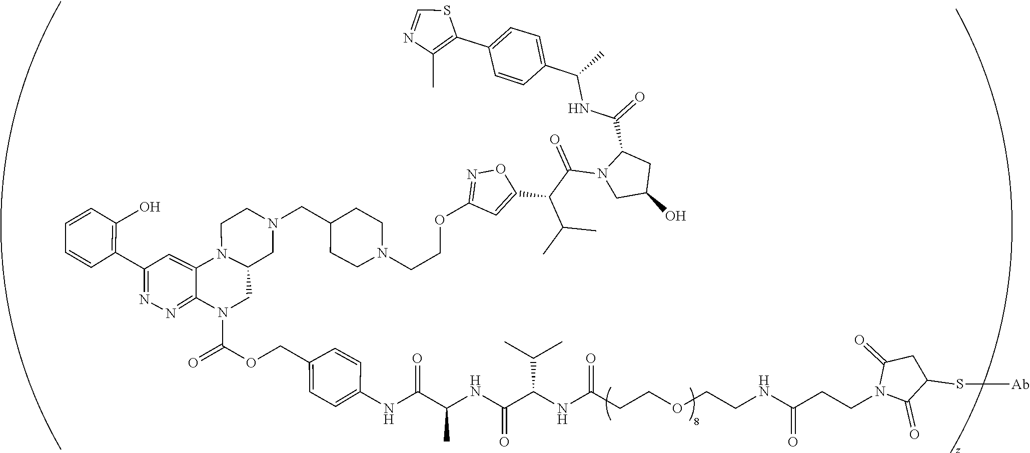 |
| 1.31 | 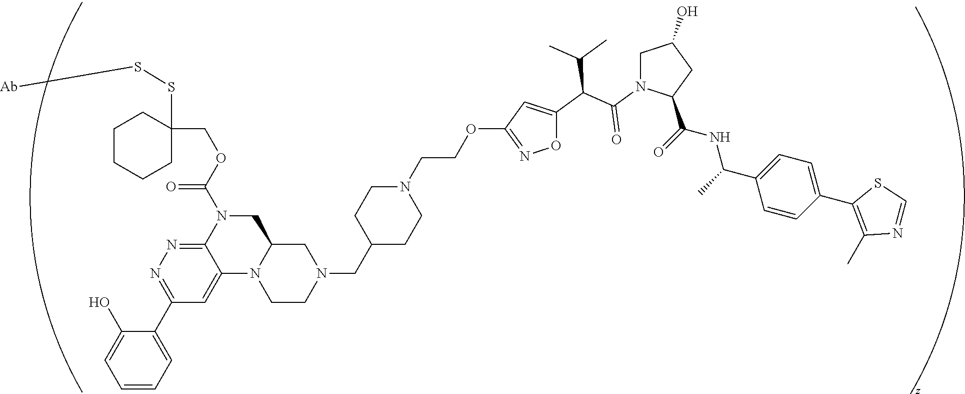 |
| 1.32 | 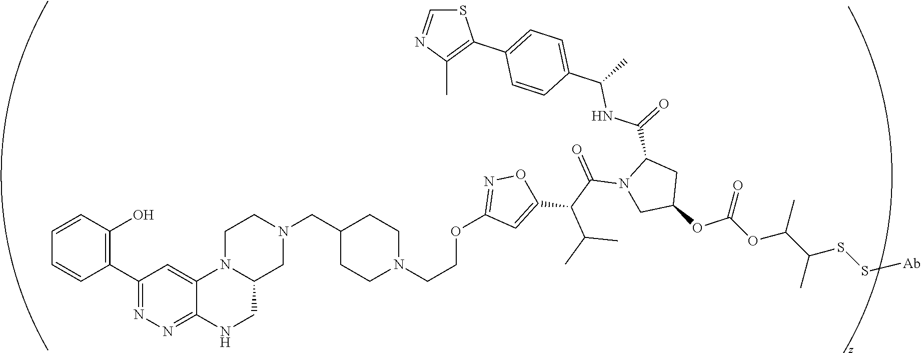 |
| 1.33 | 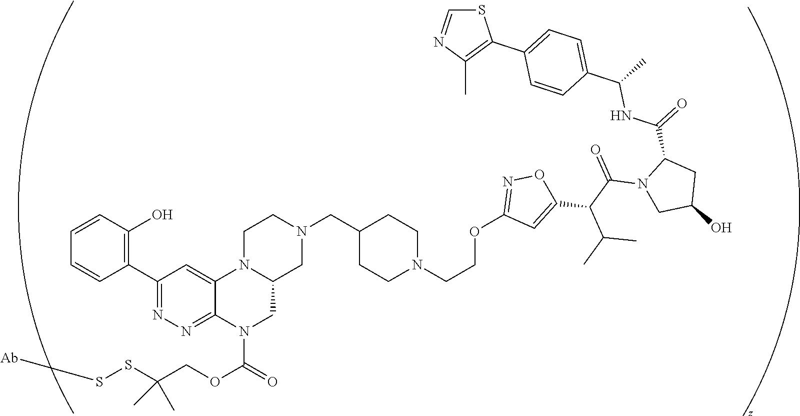 |
| 1.34 | 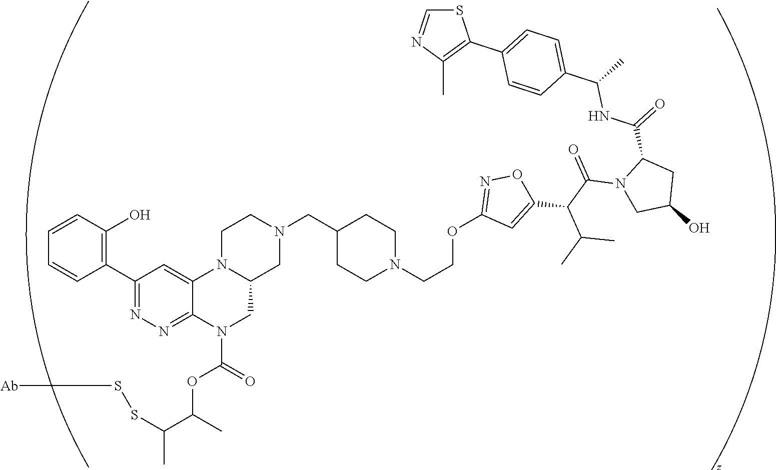 |
| 1.35 | 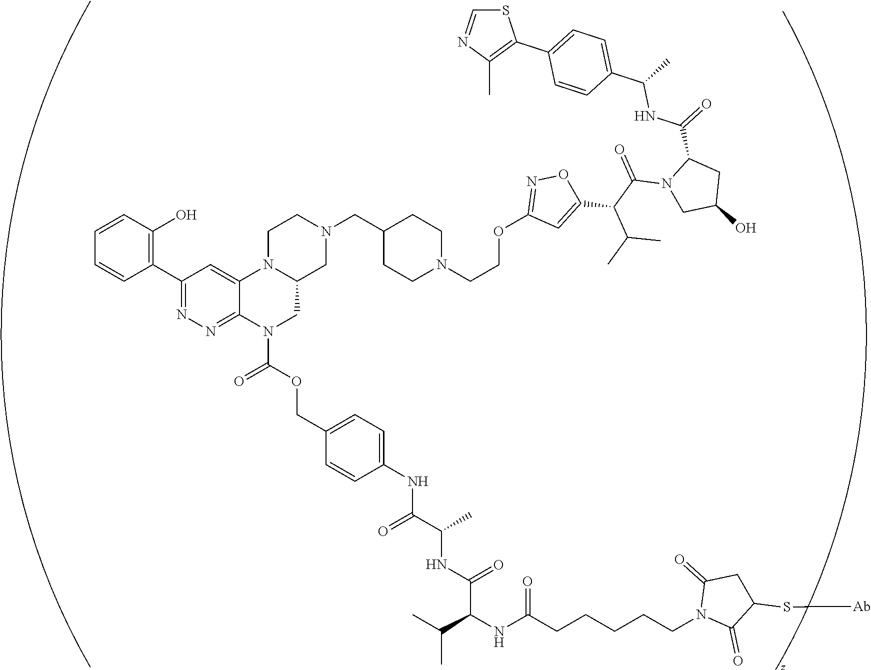 |
| 1.36 | 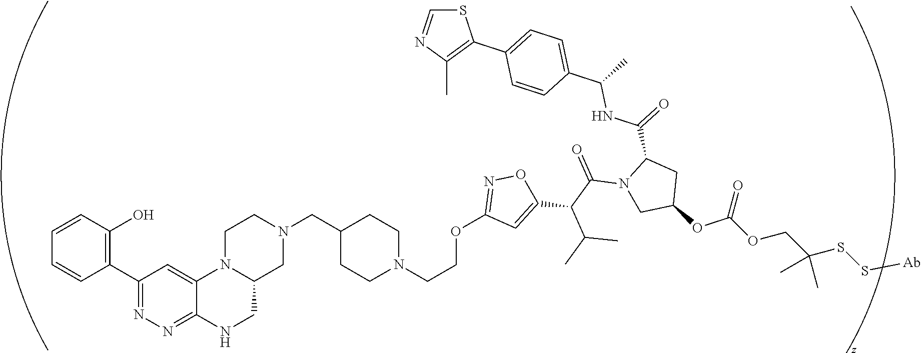 |
| 1.37 | 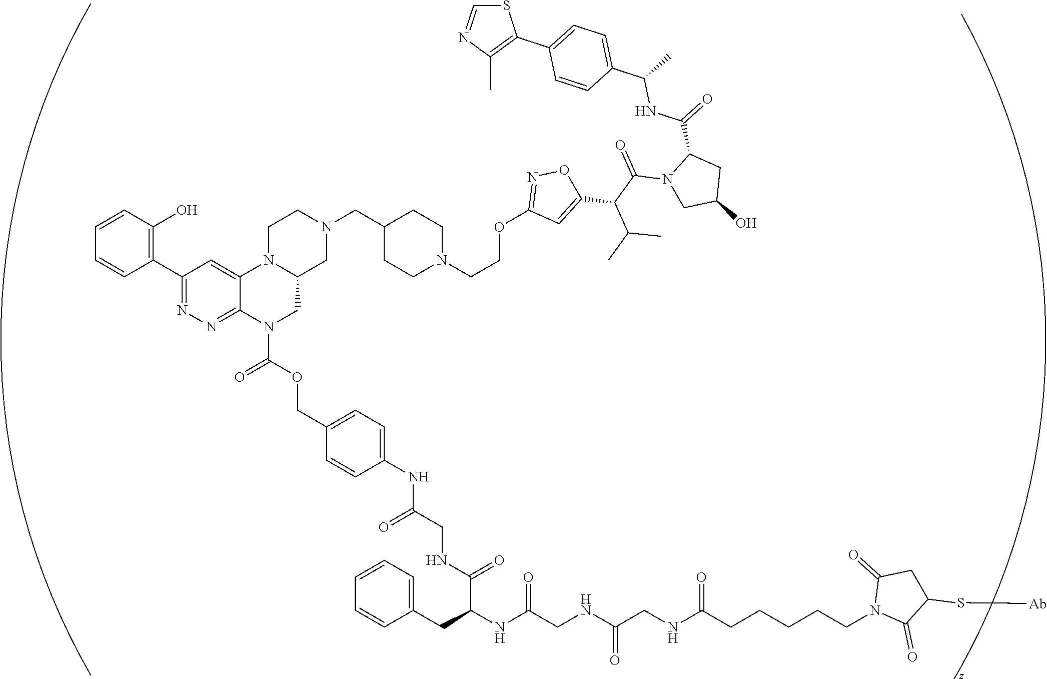 |
| 1.38 | 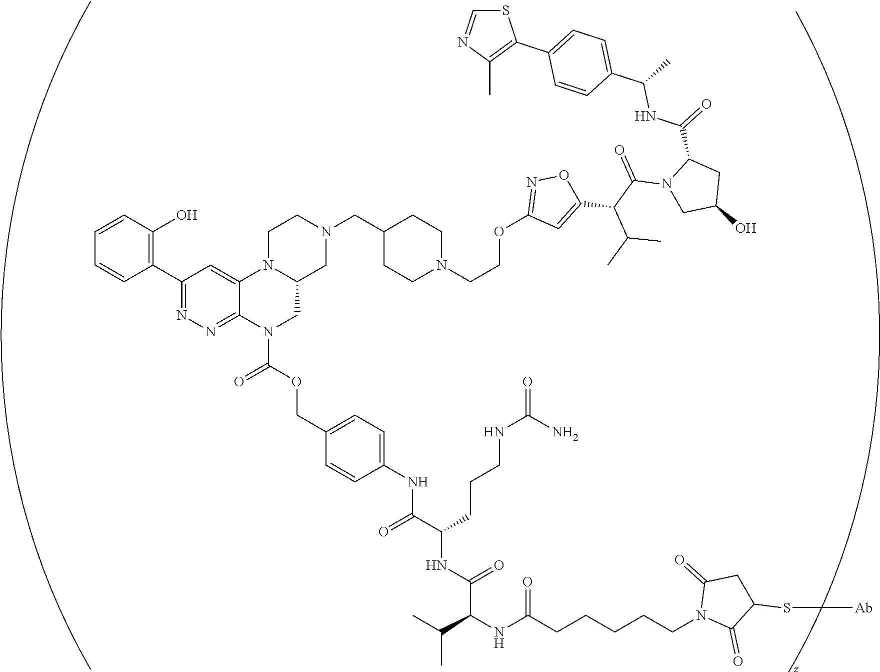 |
| 1.39 | 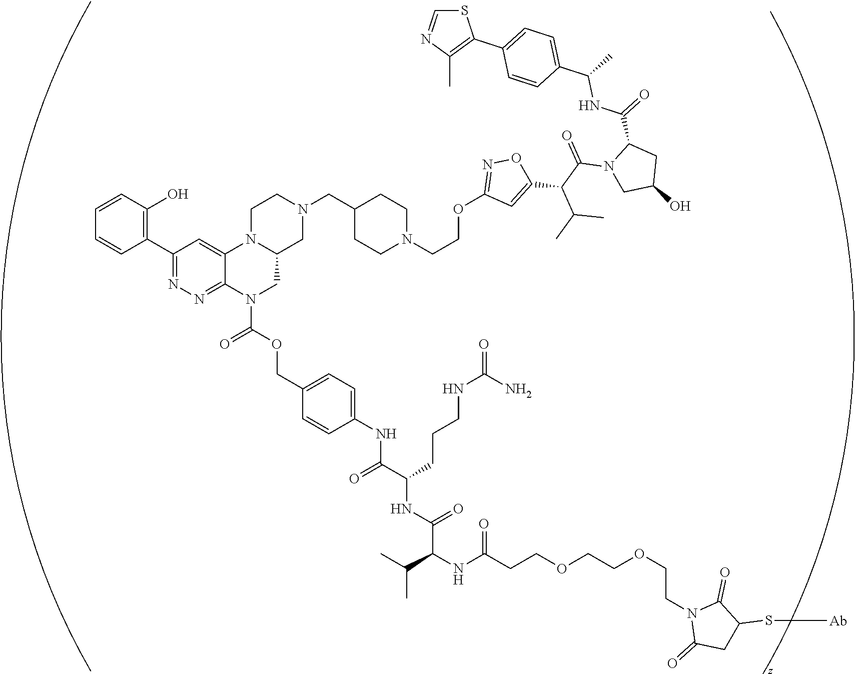 |
| 1.40 | 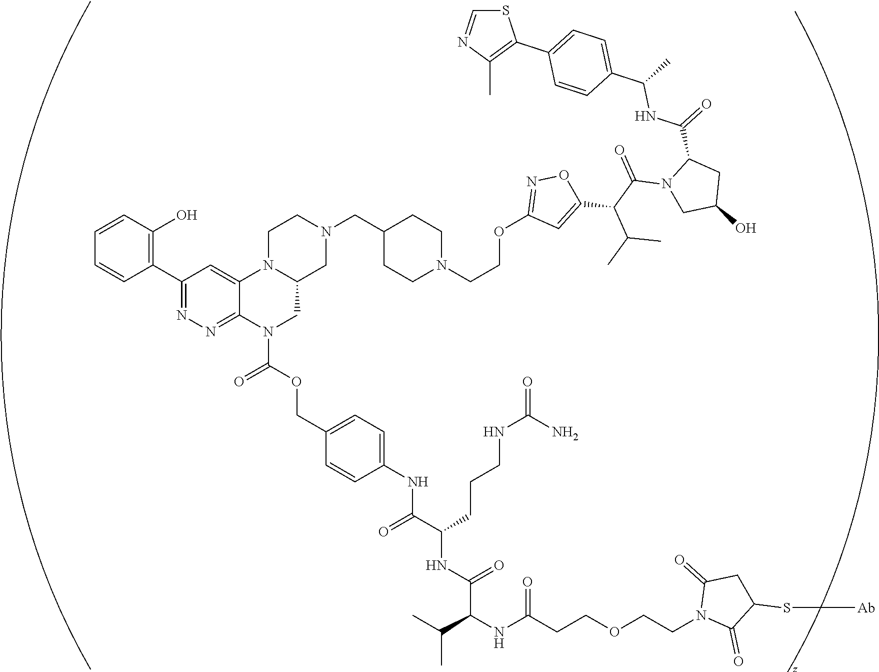 |
| 1.41 | 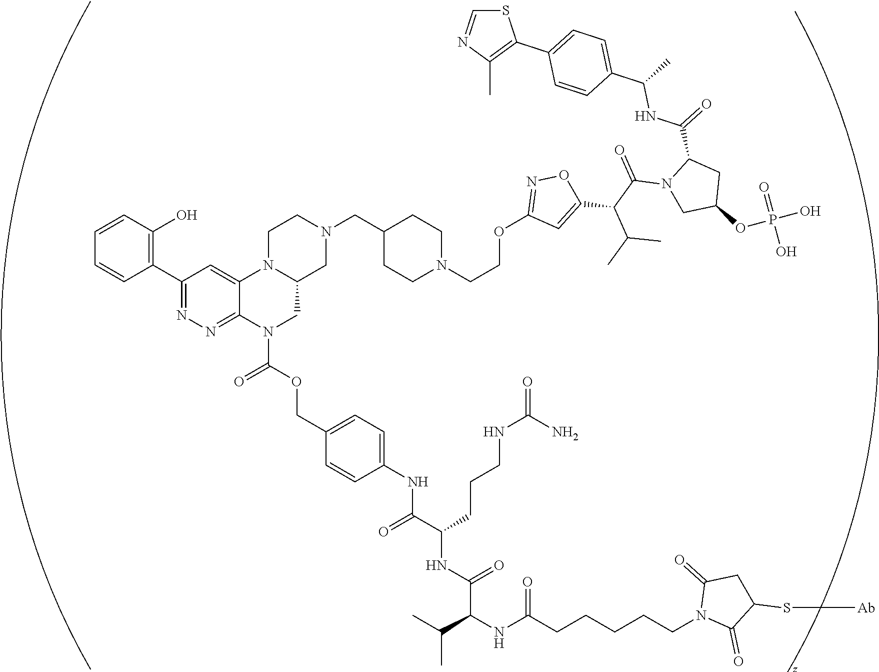 |
| 1.42 | 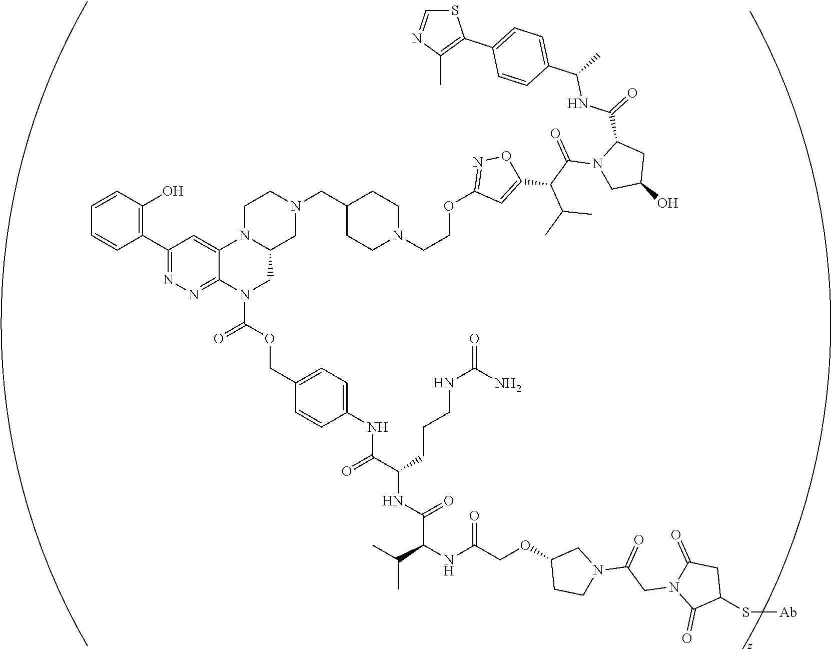 |
| 1.43 | 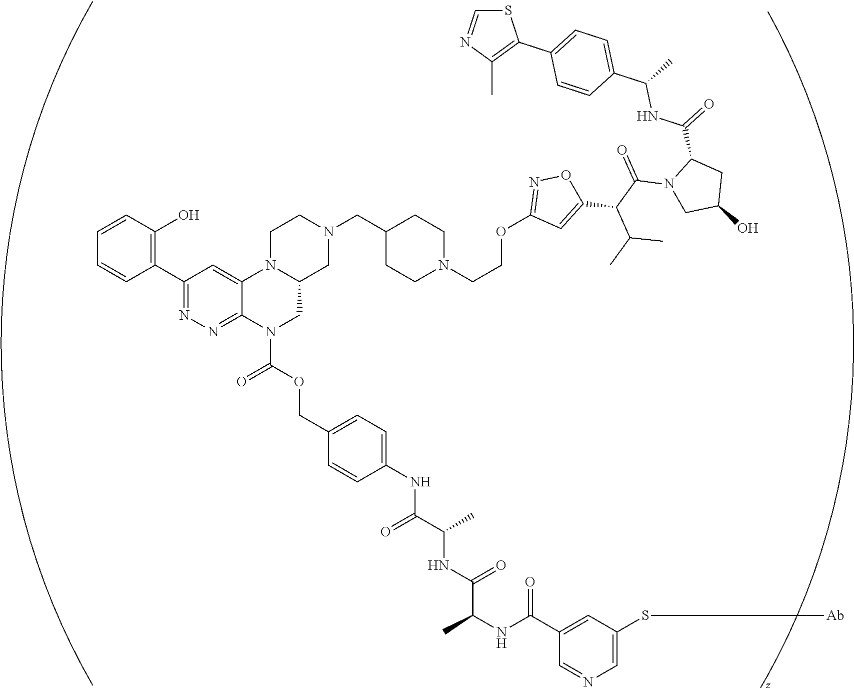 |
| 1.44 | 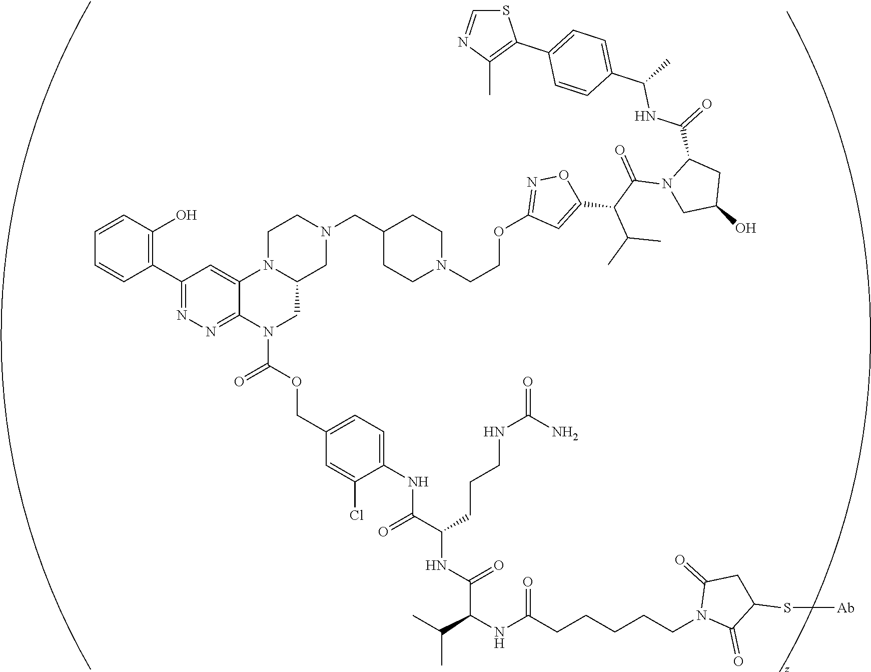 |
| 1.45 | 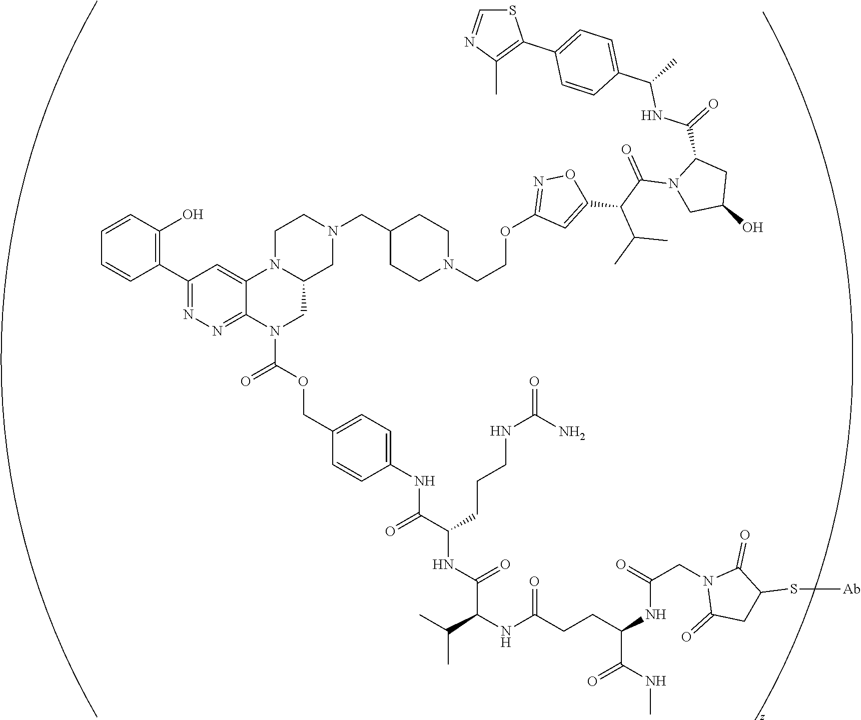 |
| 1.46 | 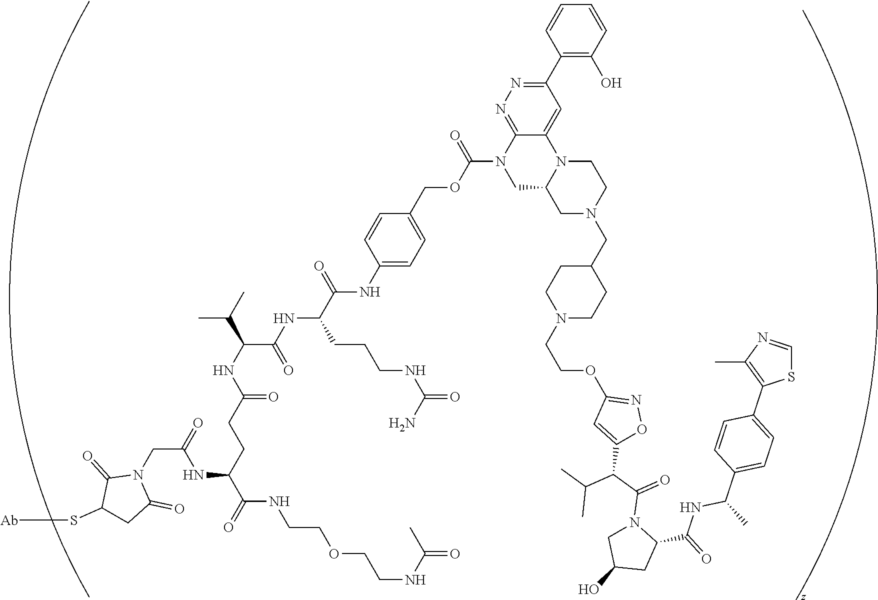 |
| 1.47 | 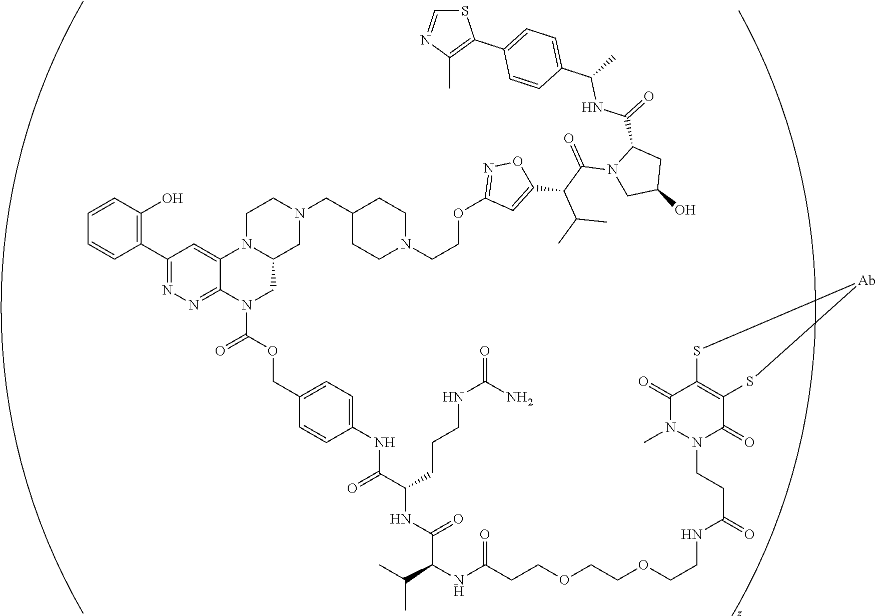 |
| 1.48 | 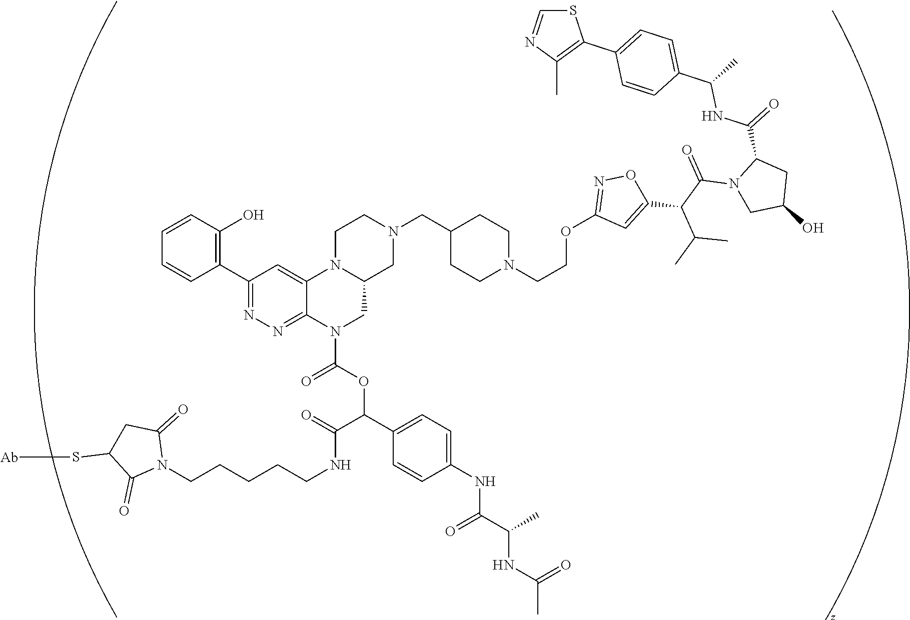 |
| 1.49 | 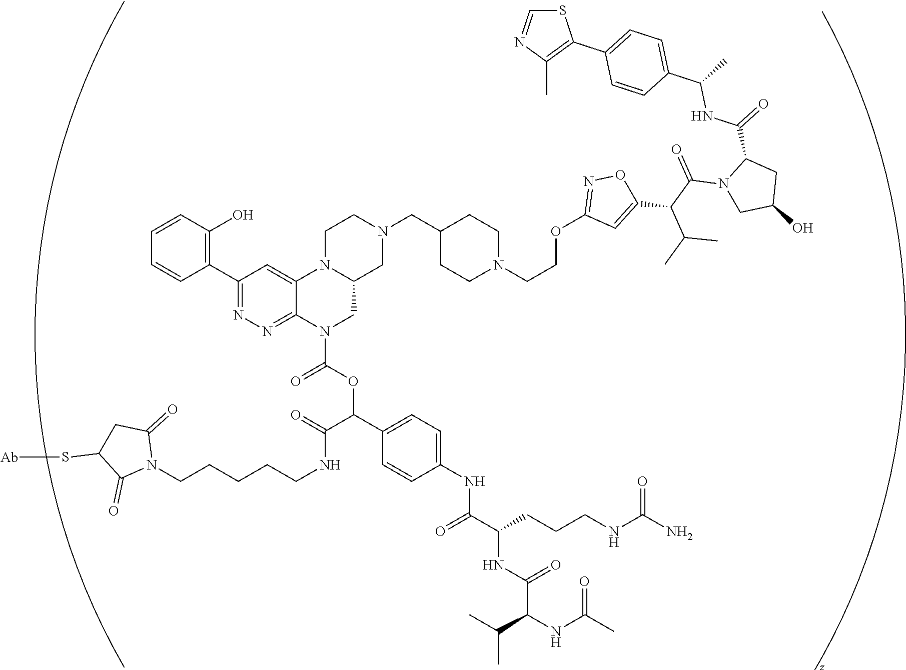 |
| 1.50 | 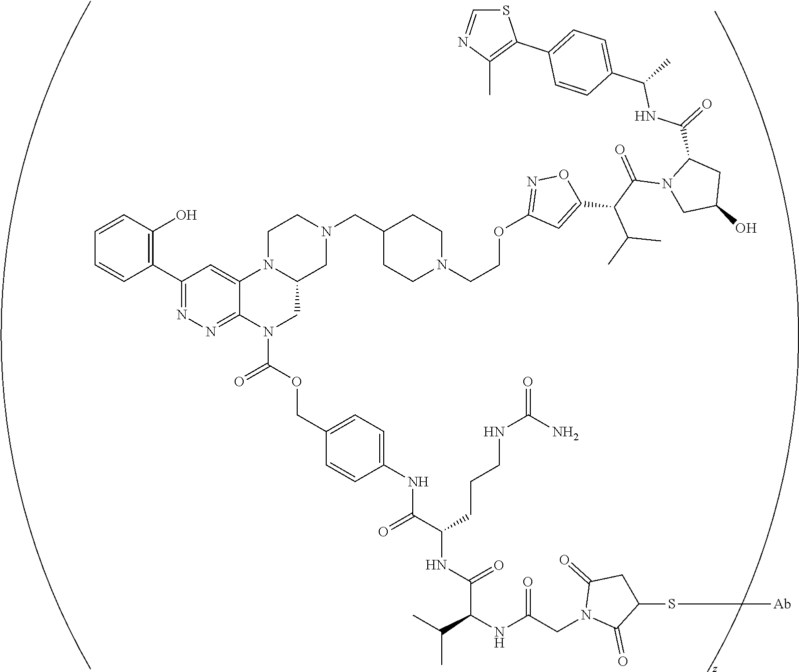 |
| 1.51 | 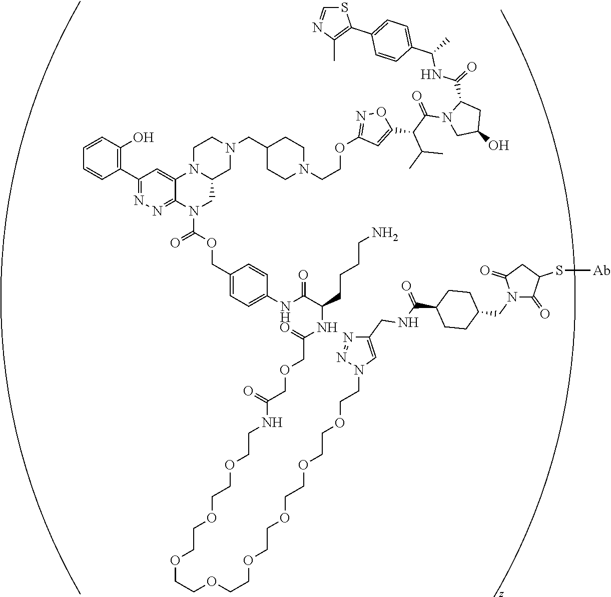 |
| 1.52 | 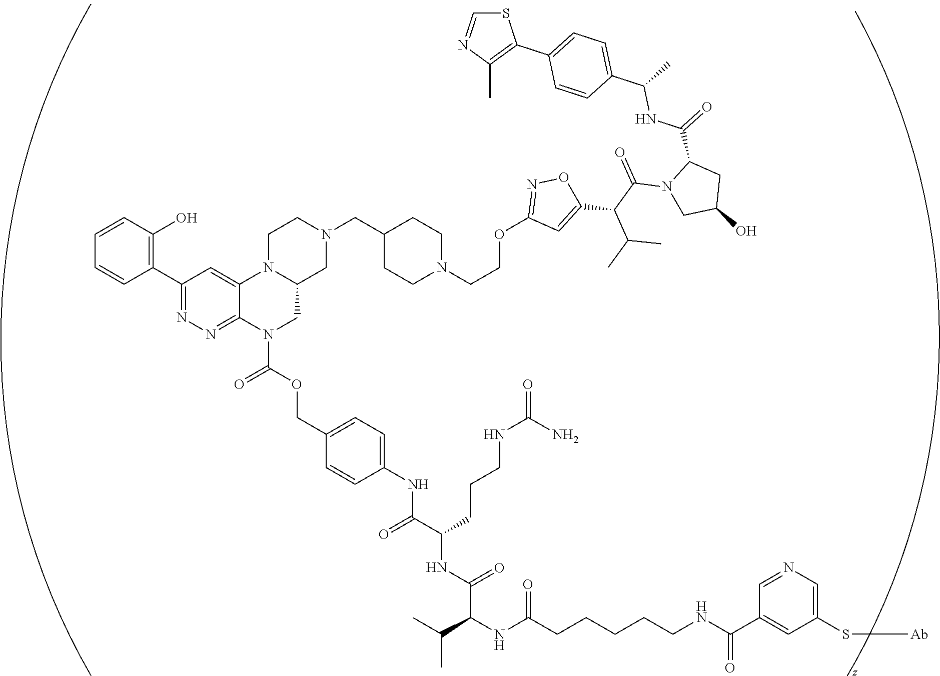 |
| 1.53 | 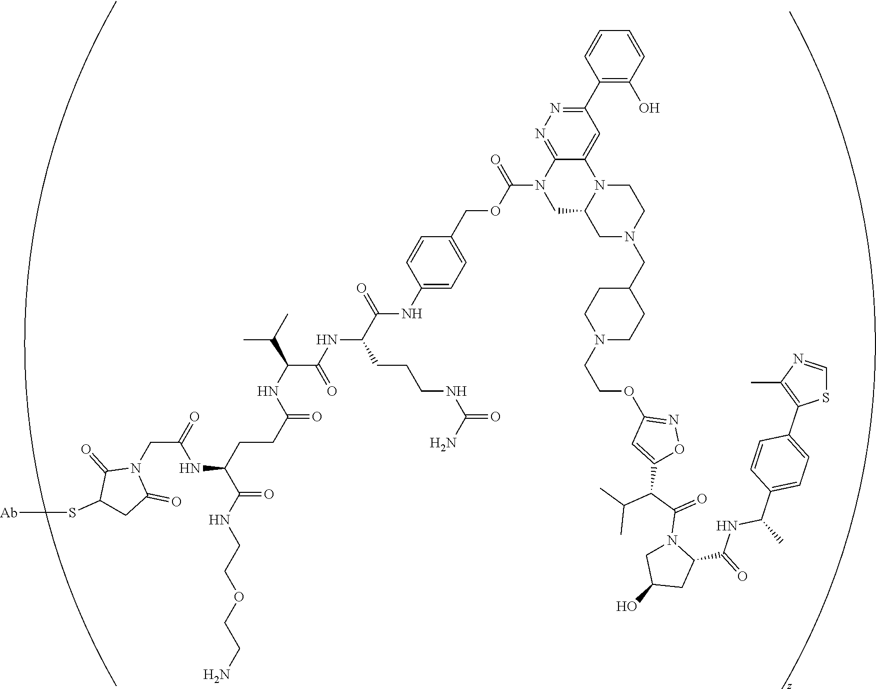 |
| 1.54 | 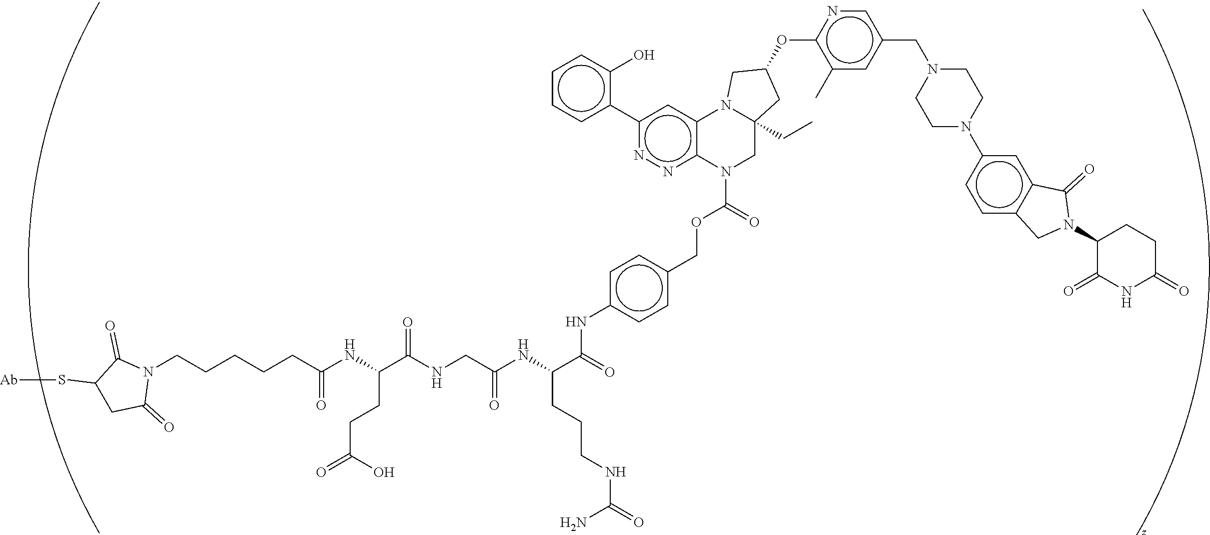 |
| 1.55 | 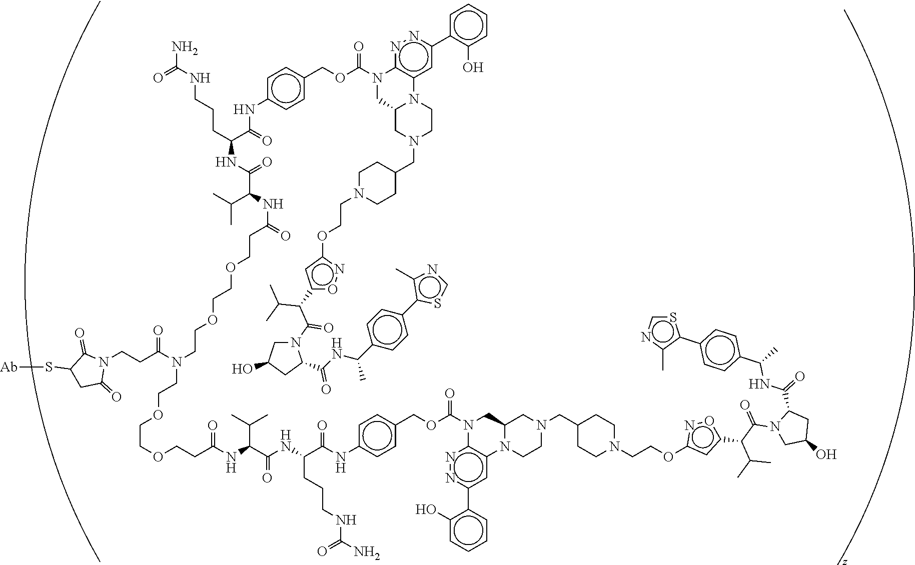 |
| 1.56 | 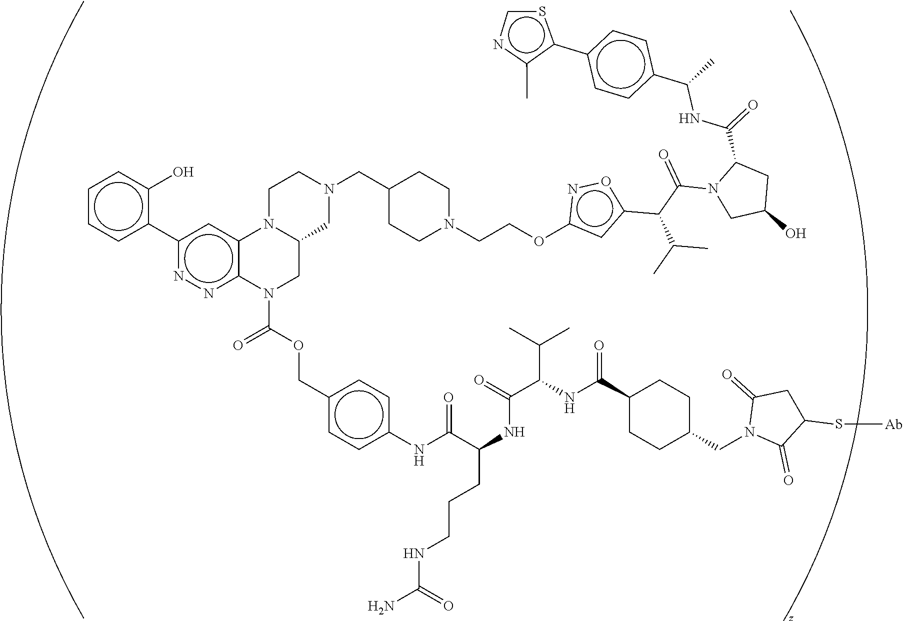 |
| 1.57 | 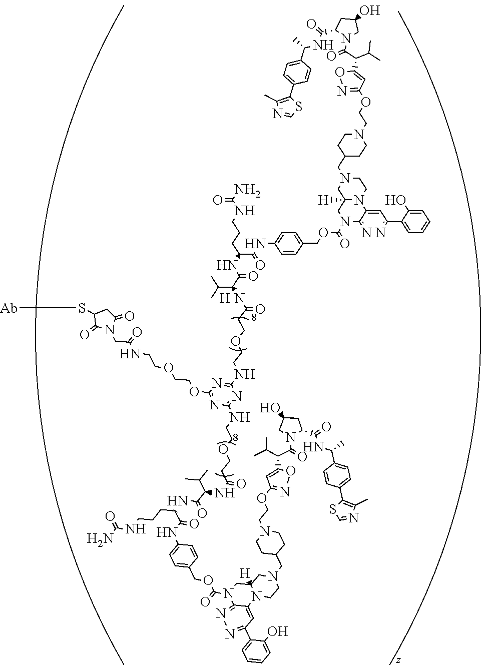 |
| 1.58 | 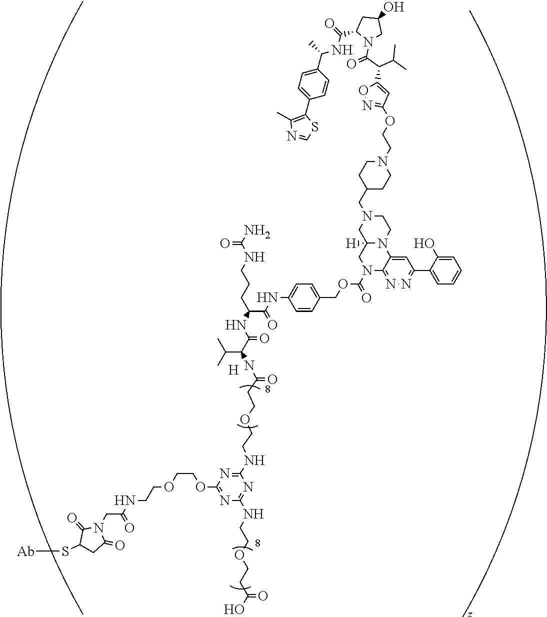 |
| 1.59 | 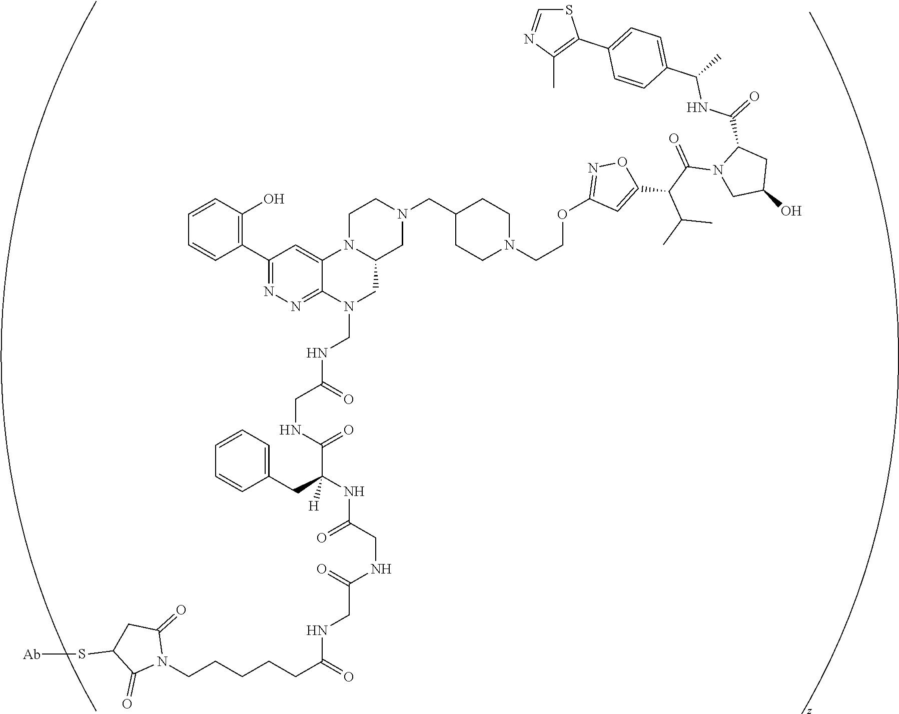 |
| 1.60 | 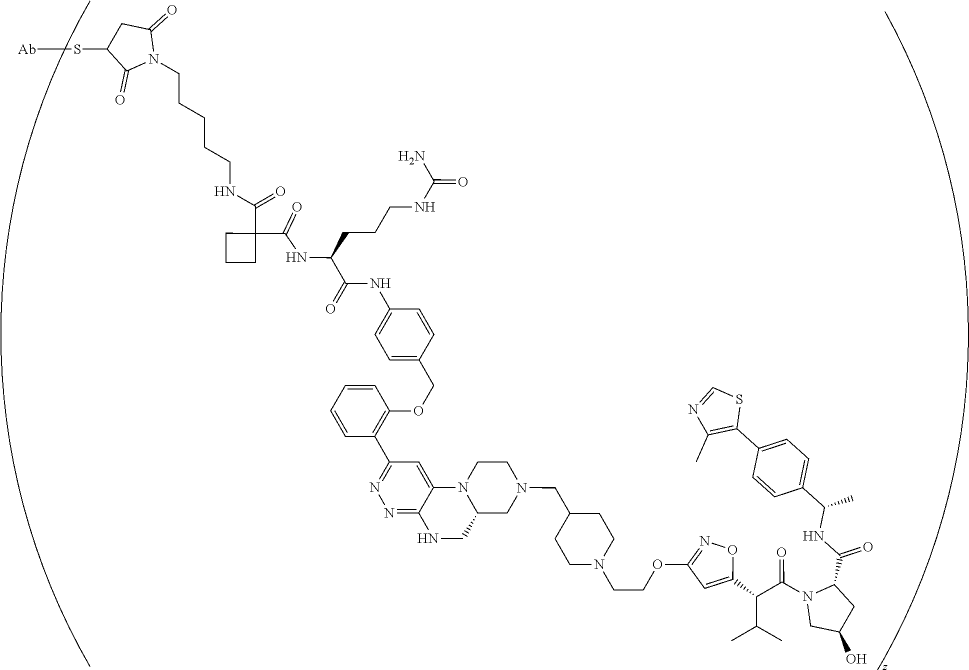 |
| 1.61 | 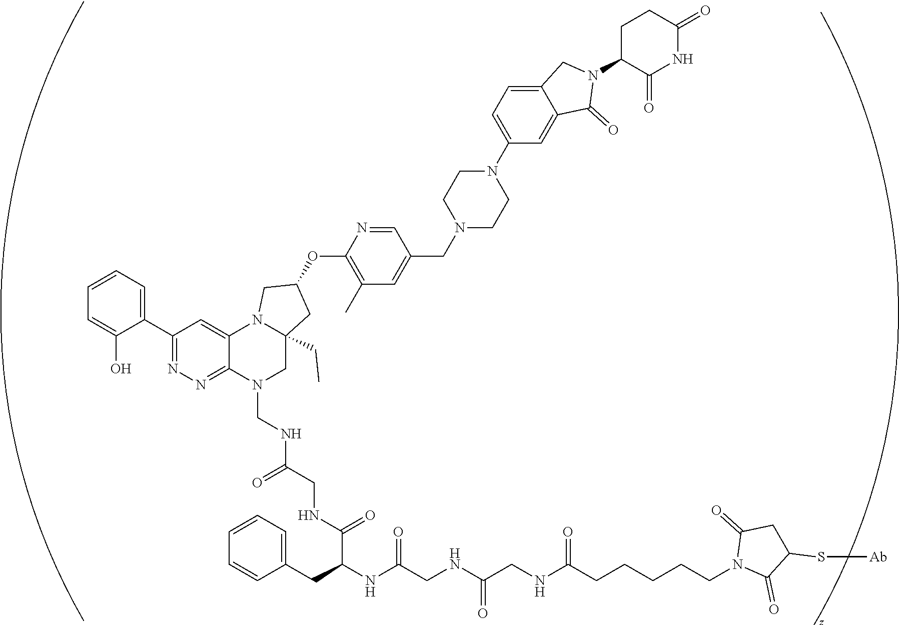 |
| 1.62 | 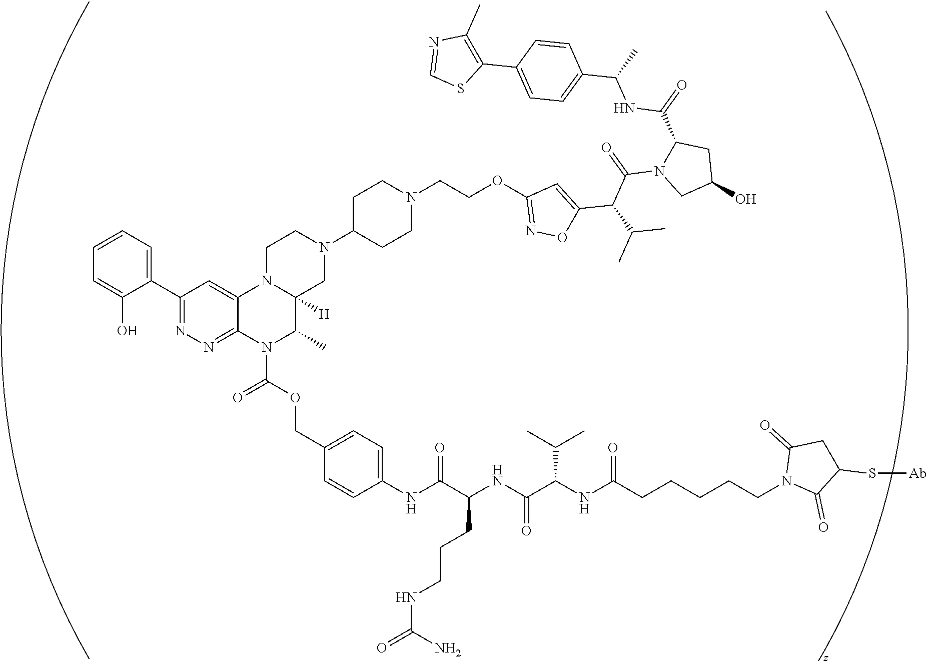 |
| 1.63 | 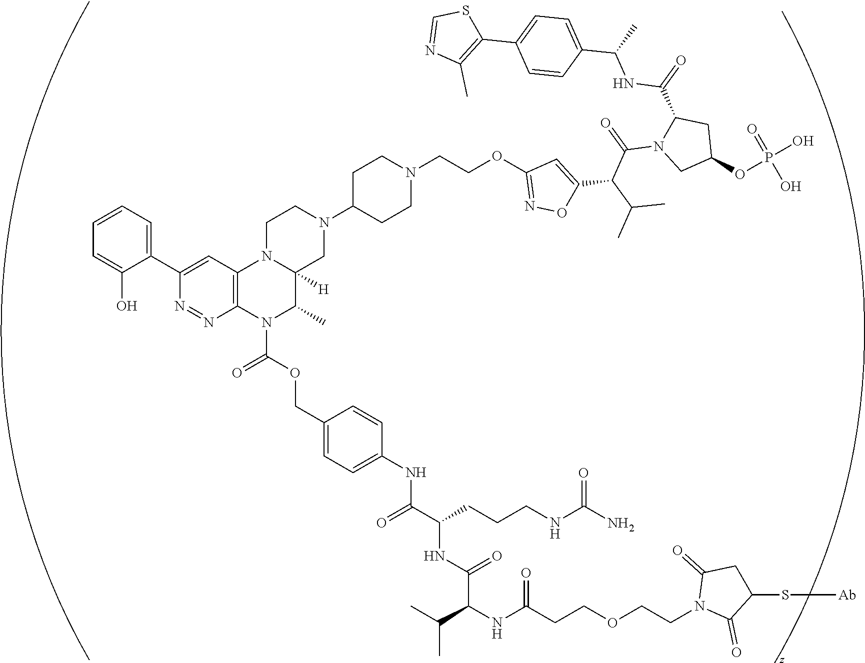 |
| 1.64 | 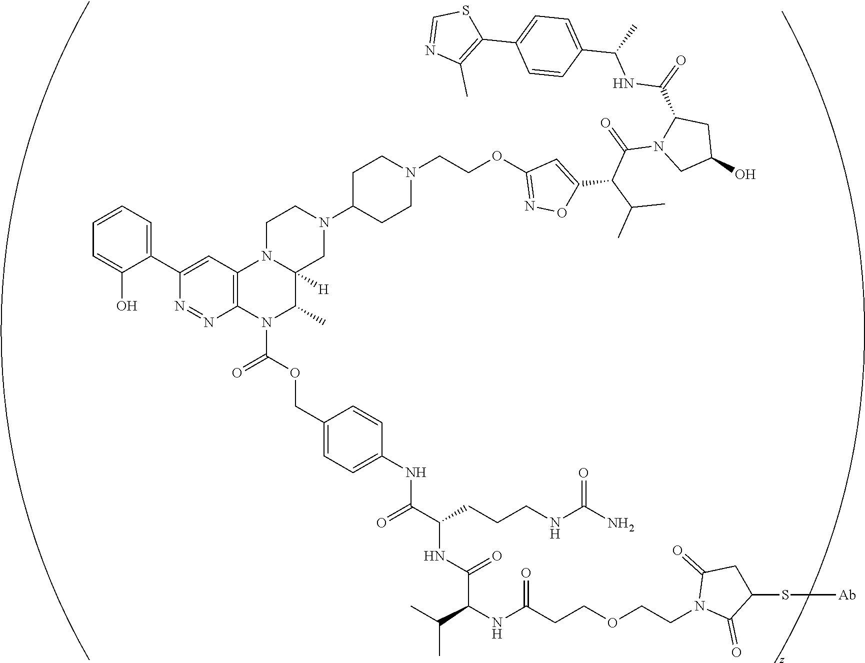 |
| 1.65 | 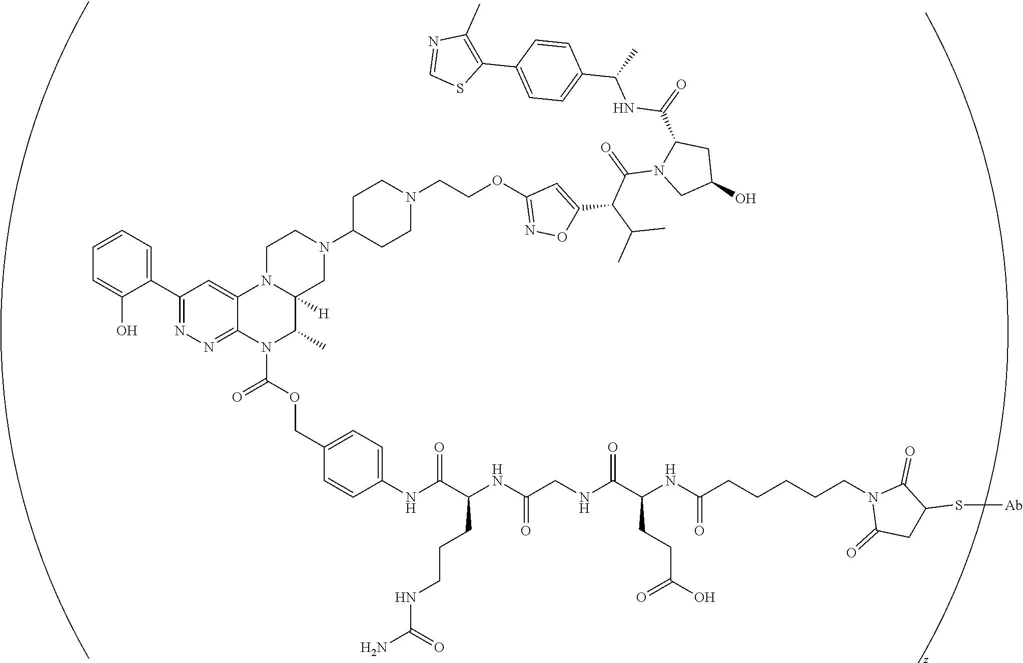 |
| 1.66 | 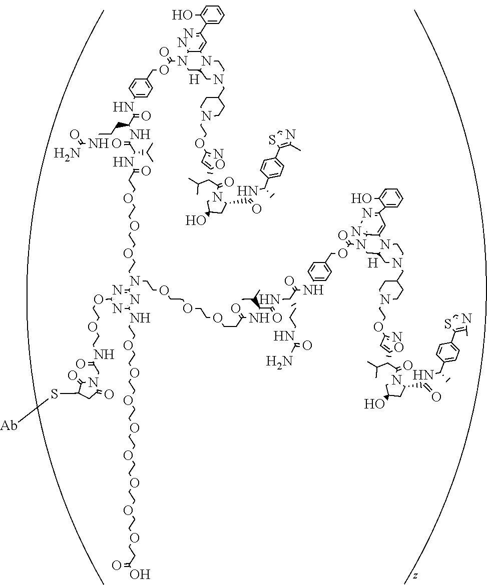 |
| 1.67 | 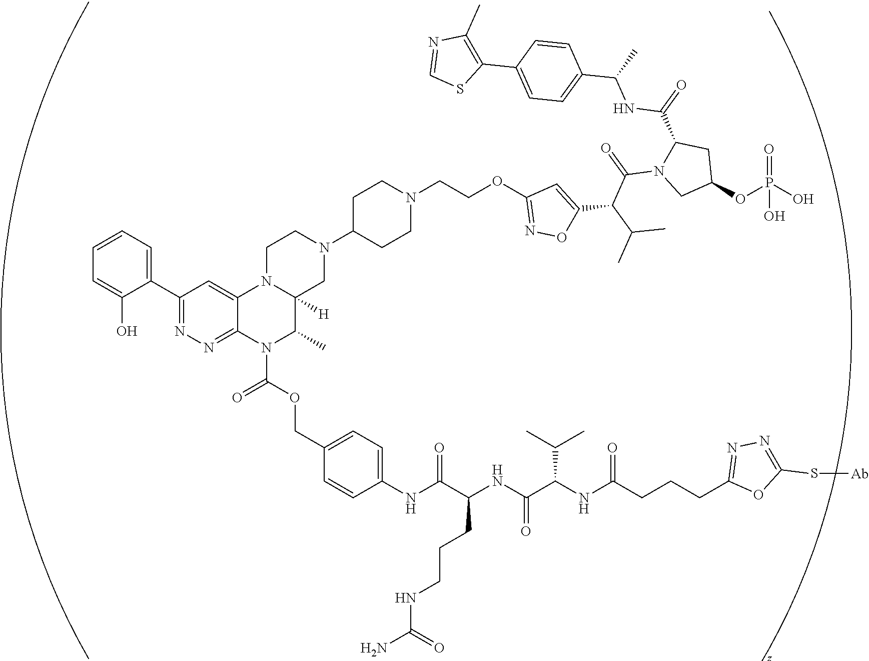 |
| 1.68 | 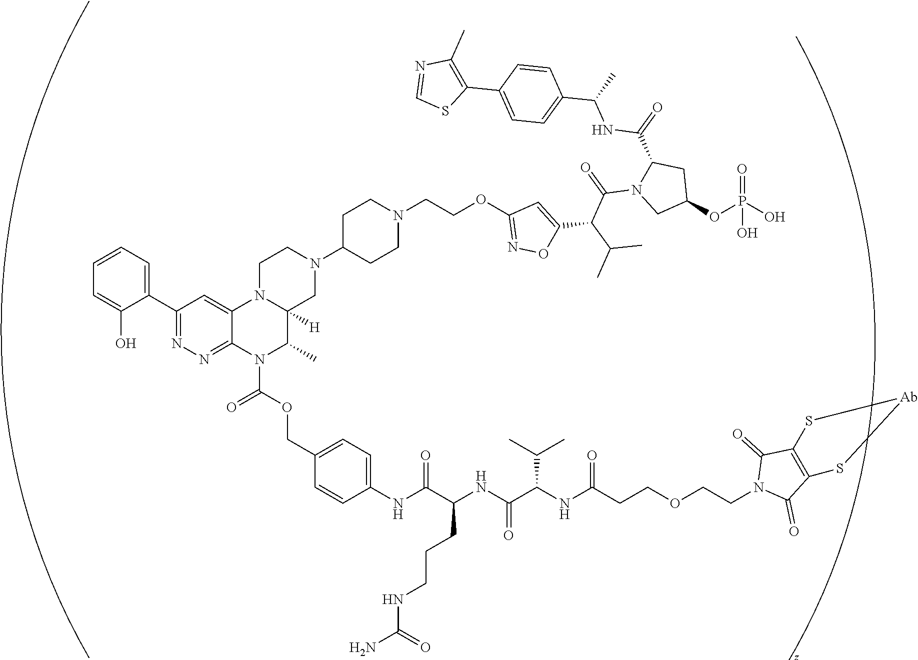 |
| 1.69 | 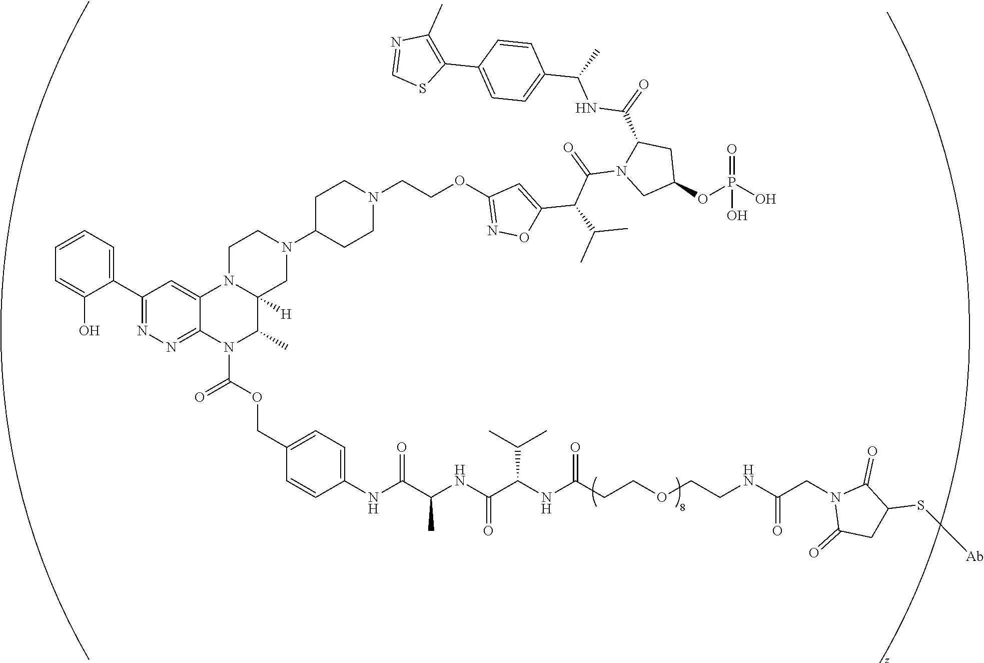 |
| 1.70 | 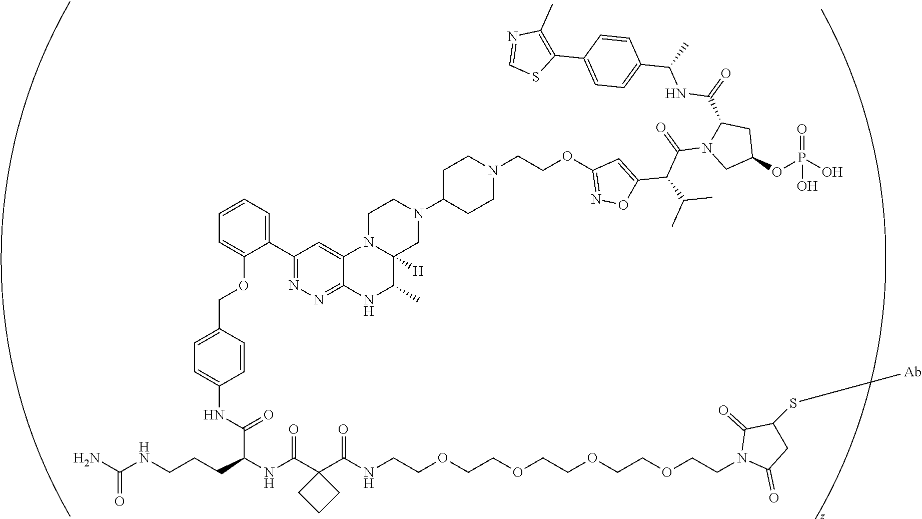 |
| 1.71 | 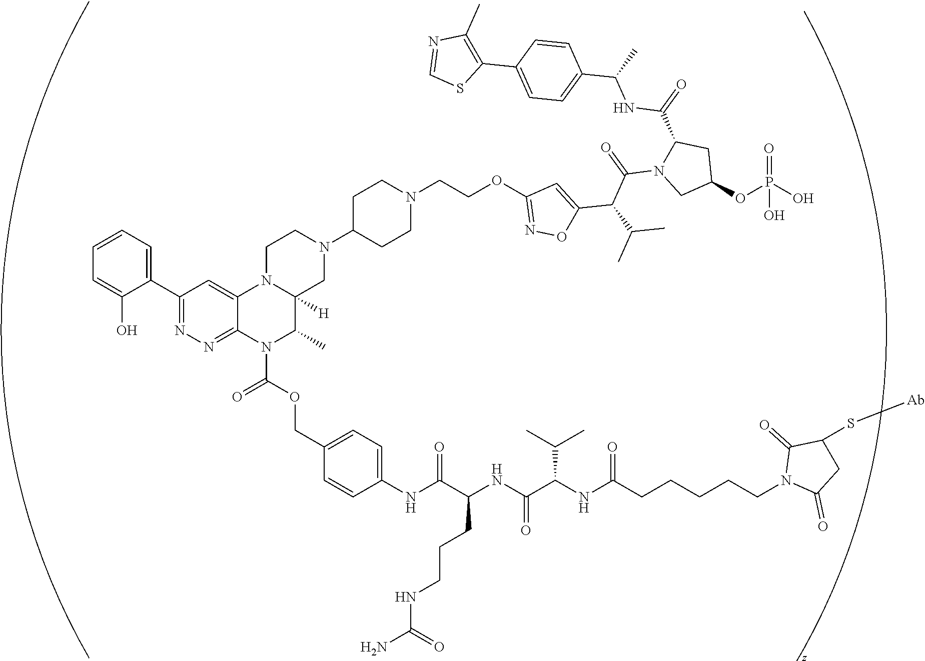 |
| 1.72 | 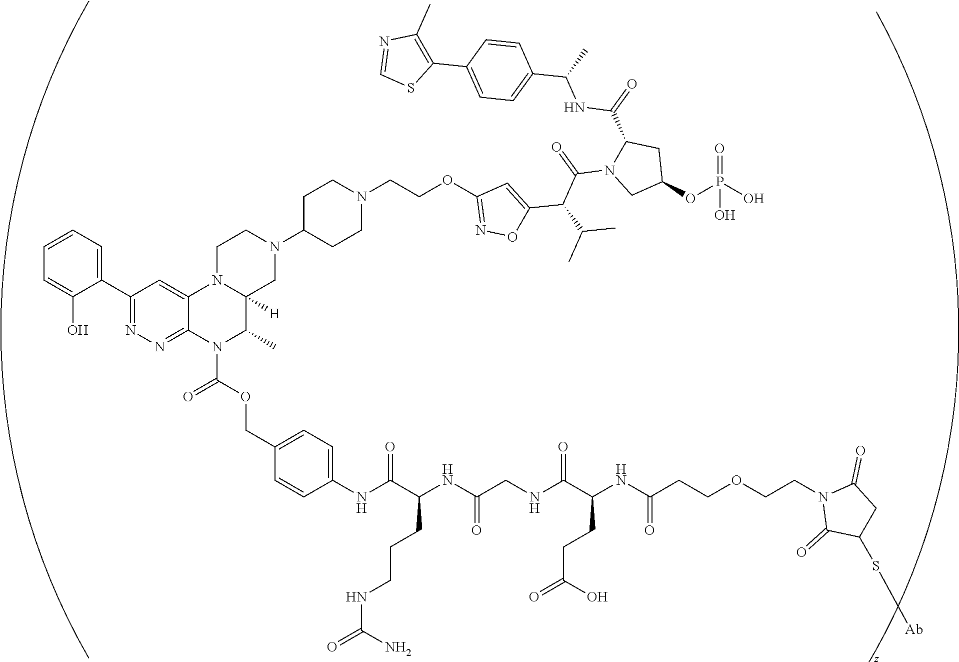 |
| 1.73 | 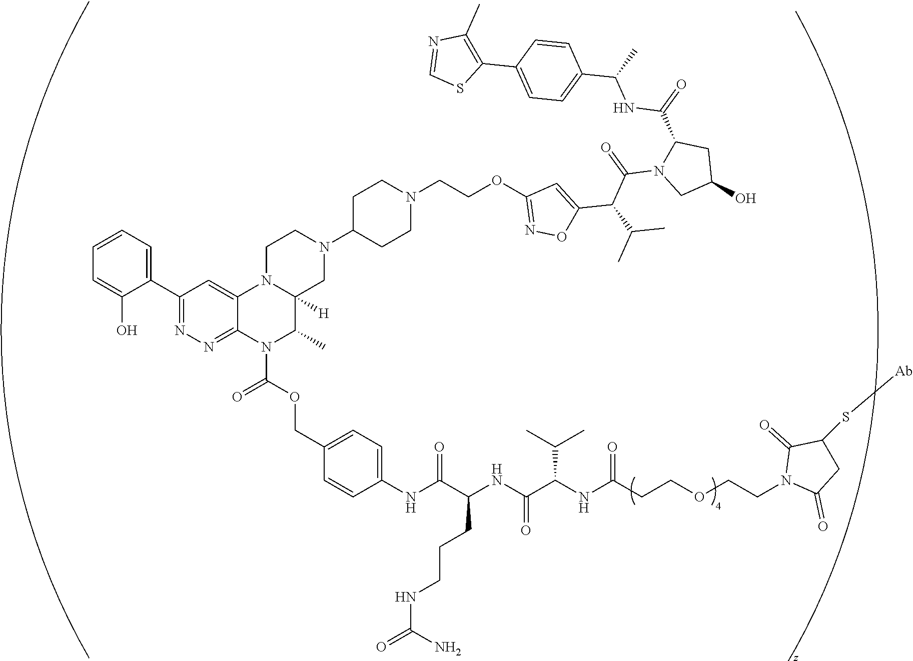 |
| 1.74 | 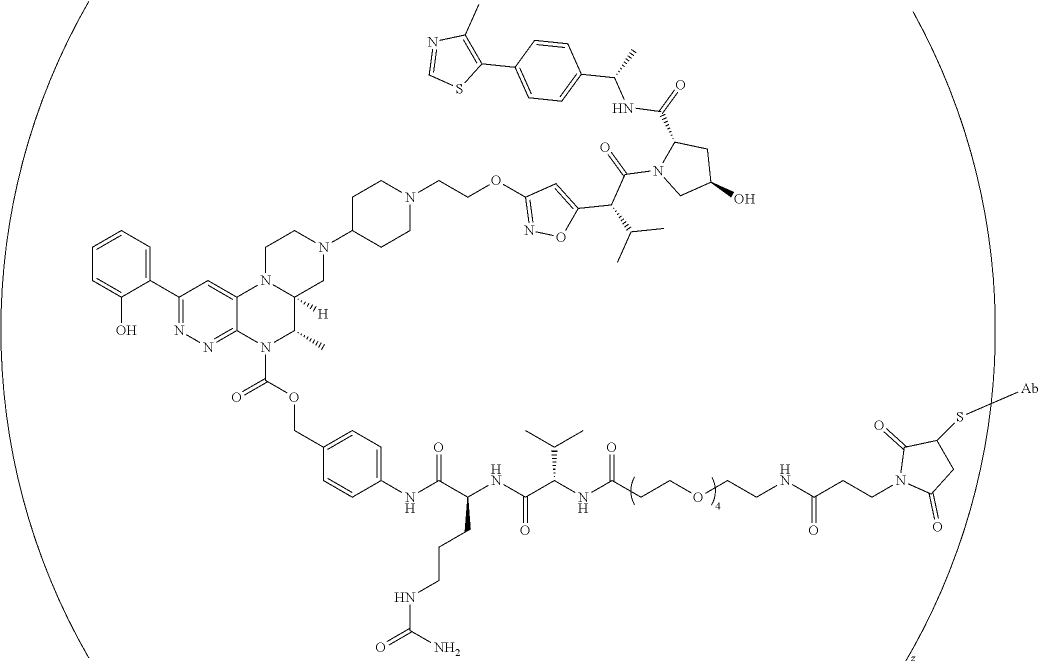 |
| 1.76 | 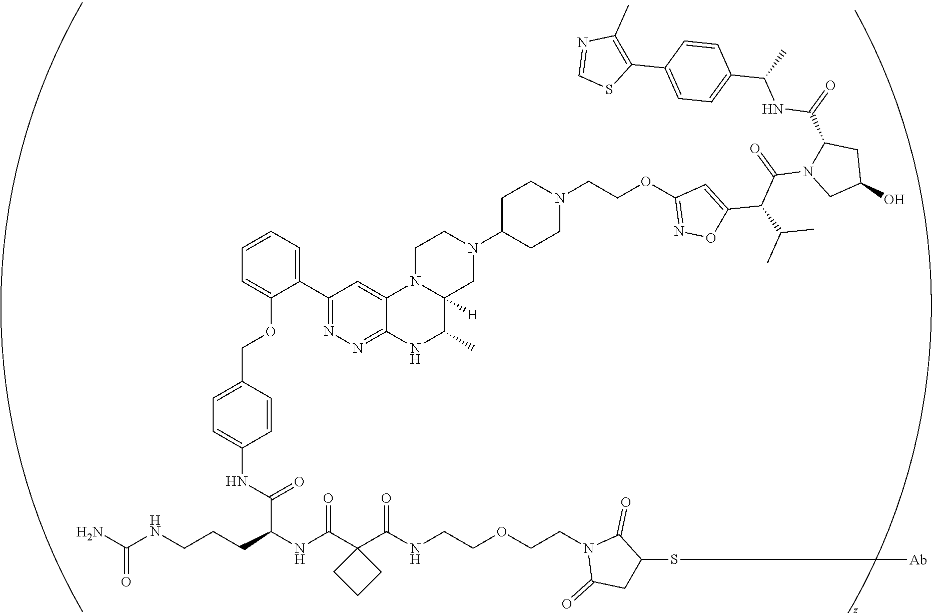 |
| 1.77 | 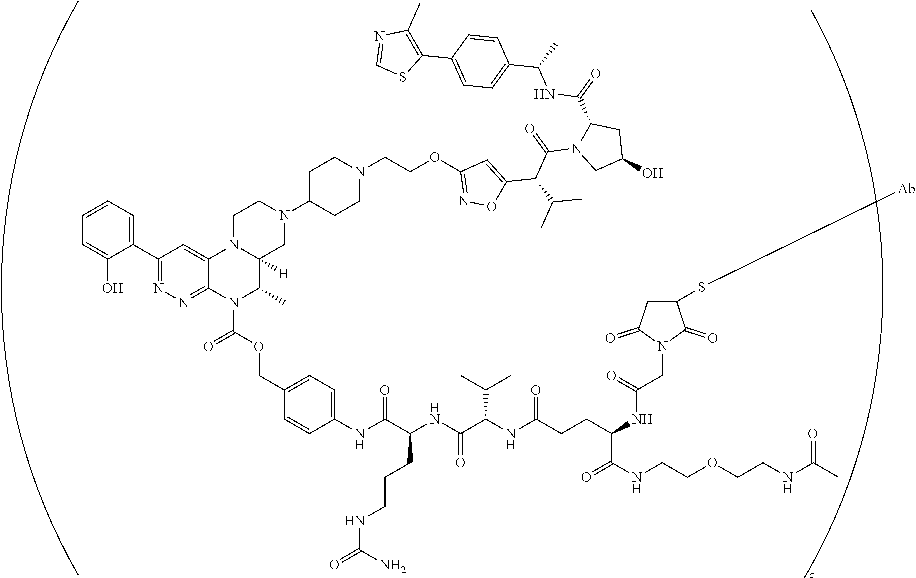 |
| 1.78 | 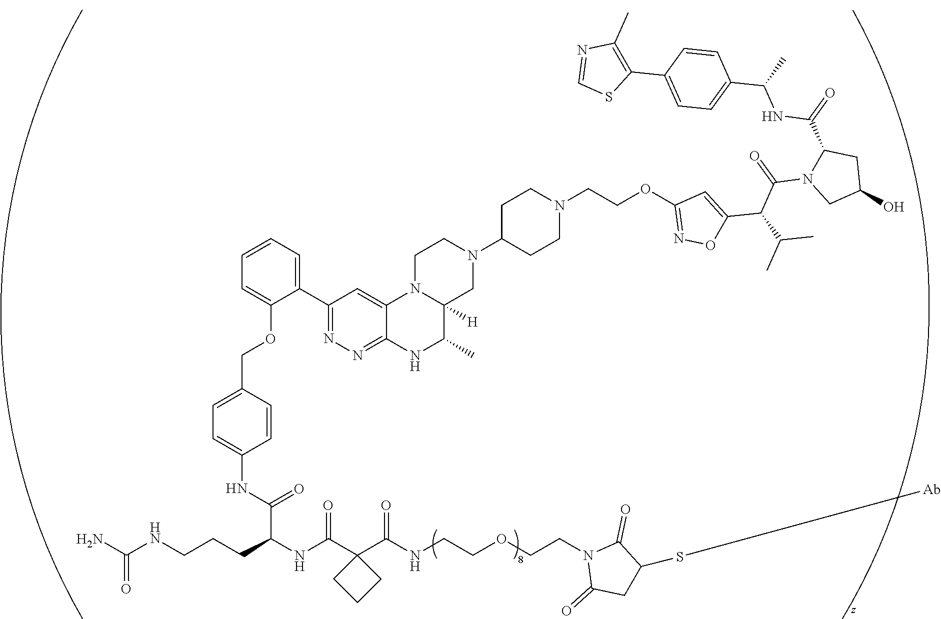 |
| 1.79 | 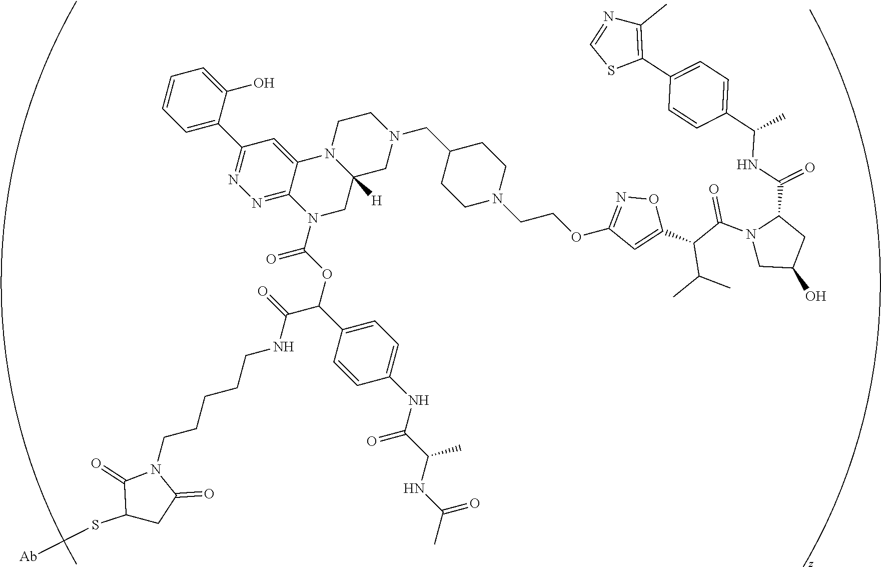 |
| 1.80 | 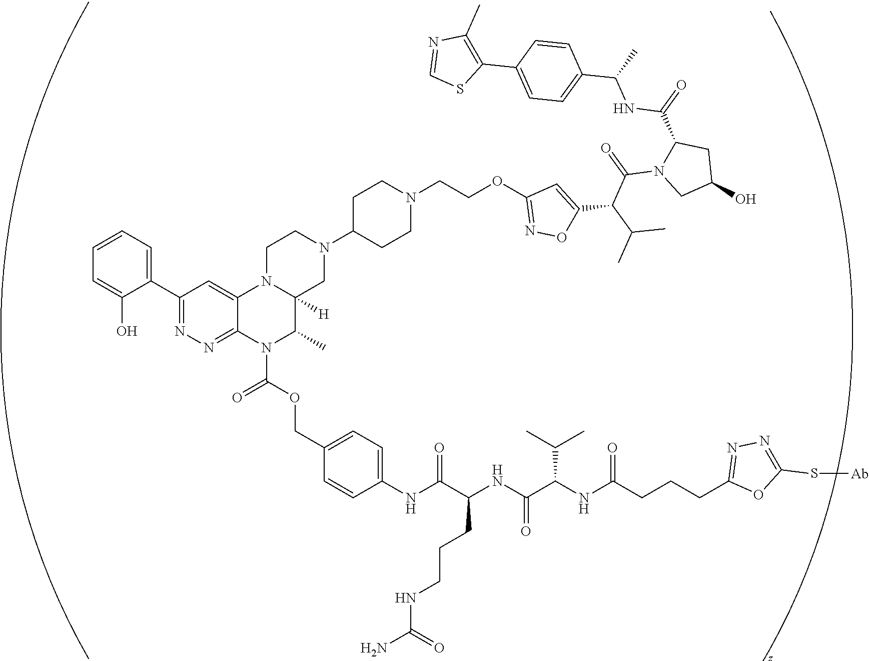 |
| 1.81 | 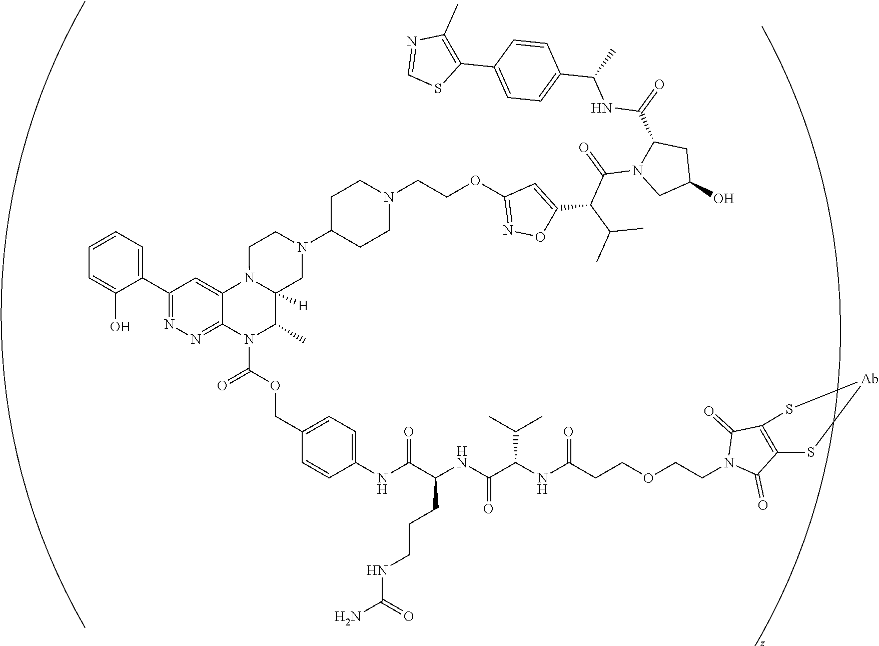 |
| 1.82 | 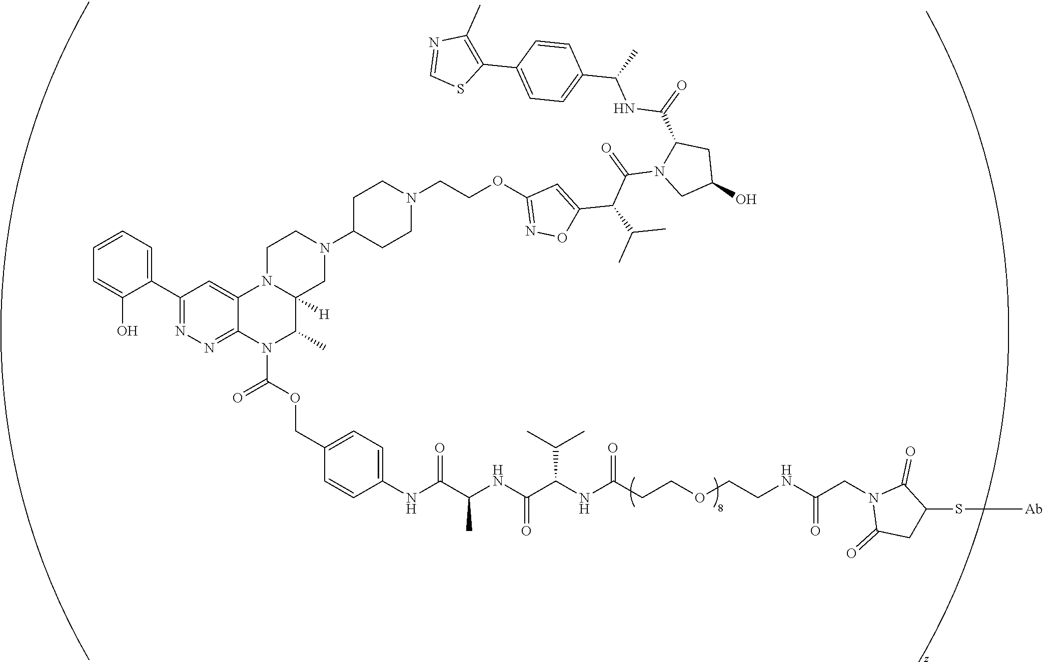 |
| 1.83 | 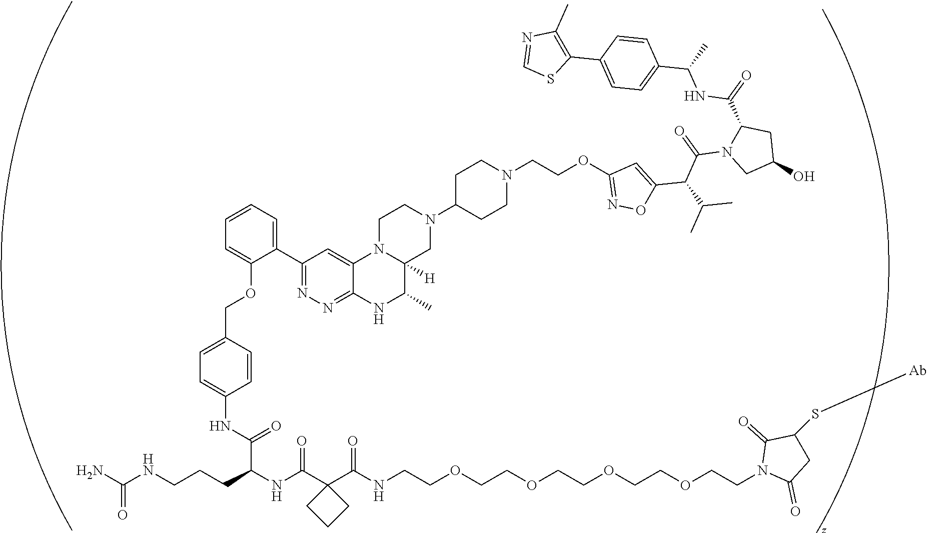 |
| 1.84 | 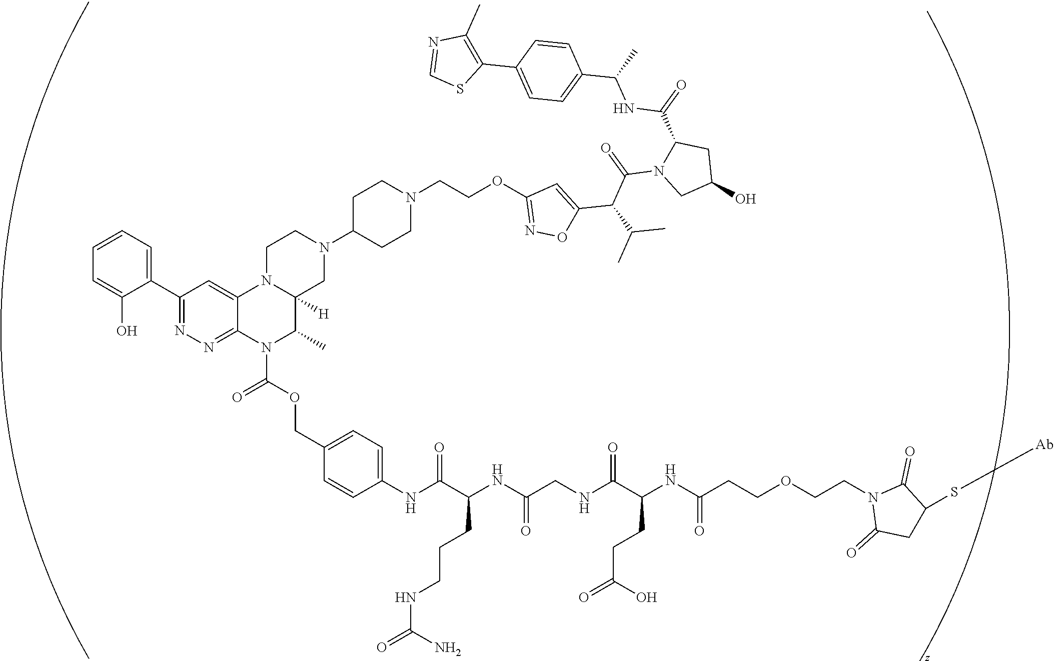 |
| 1.85 | 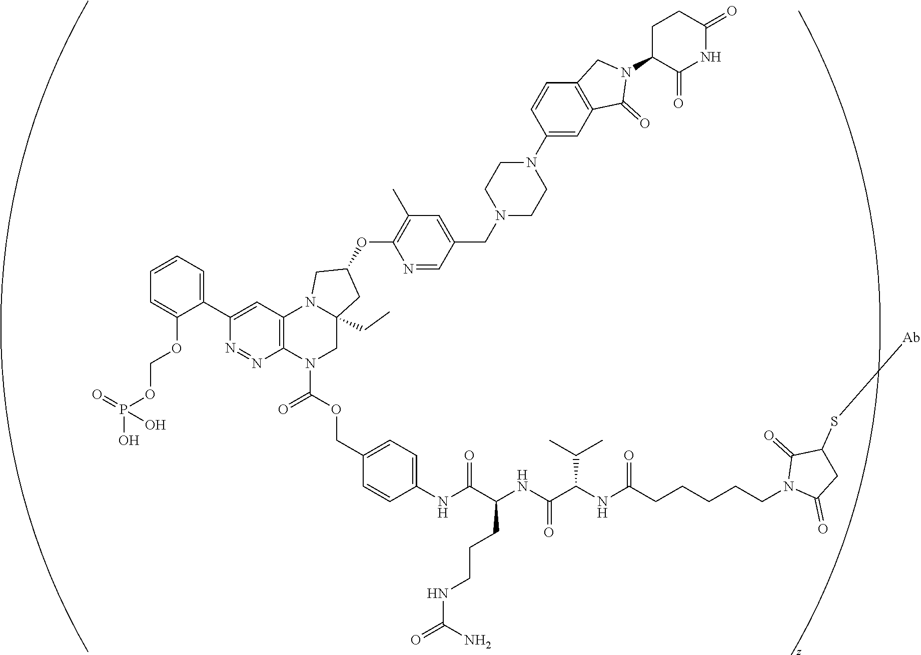 |

### II. Drug-Linker Compounds

In some embodiments, it is desirable to synthesize a drug-linker compound, also referred to as “a degrader-linker” or a “degrader-linker compound” herein, before conjugating an antibody and forming an antibody-drug conjugate. In some embodiments, a drug-linker compound is an intermediate. In some embodiments, the linker of a drug-linker compound comprises a reactive group for conjugating the linker to the antibody, or antigen-binding fragment thereof. In some embodiments, a drug-linker compound comprises a degrader compound (D) as described herein and a linker precursor (L′) as described herein. In some embodiments, a drug-linker compound has the structure:

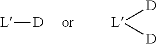

wherein D is a degrader compound and L′ is a linker precursor.

It is to be understood that all of the embodiments of the linkers (L) of Antibody Drug Conjugate compounds described herein apply to drug-linker compounds described herein wherein the M group described for the Antibody Drug Conjugate compounds is replaced with M′. It is also to be understood that all of the embodiments of the of Antibody Drug Conjugate compounds described herein apply to drug-linker compounds described herein wherein the M group described for the Antibody Drug Conjugate compounds is replaced with M′.

In some embodiments, a linker precursor (L′) has the Formula (ii):

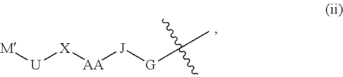

wherein
M′ is selected from the group consisting of:

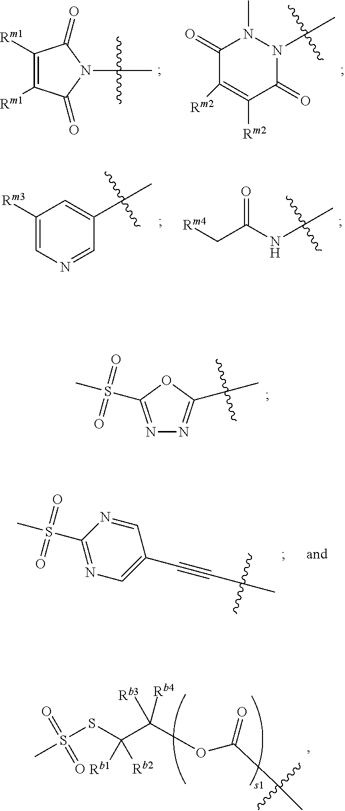

wherein
   each R^{m1} and R^{m2} is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1;
   wherein the wavy line of M′ indicates the point of covalent attachment to the remainder of the structure of L′ and the wavy line of Formula (ii) indicates the point of covalent attachment to the degrader compound (D); and
   wherein U, X, AA, J, and G are as defined herein for Antibody Drug Conjugate compounds.

In some embodiments, L′ is a linker precursor of Formula (iia):

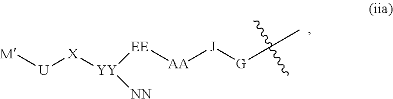

wherein
M′ is selected from the group consisting of

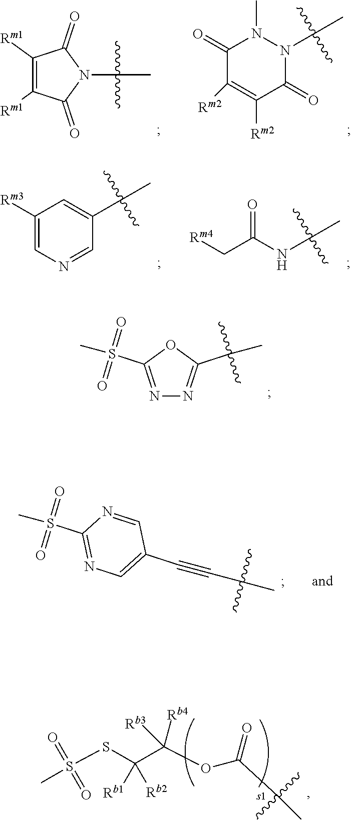

wherein
   each R^{m1} and R^{m2} is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1;
   wherein the wavy line of M′ indicates the point of covalent attachment to the remainder of the structure of L′ and the wavy line of Formula (iia) indicates the point of covalent attachment to the degrader compound (D); and
   wherein U, X, YY, NN, EE, AA, J, and G are as defined herein for Antibody Drug Conjugate compounds.

In some embodiments, L′ is a linker precursor of Formula (iib):

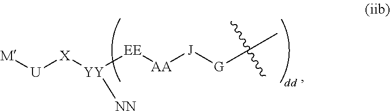

wherein
M′ is selected from the group consisting of

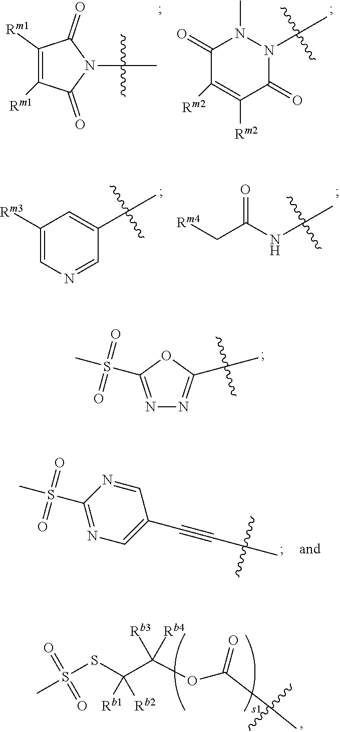

wherein
   each R^{m1} and R^{m2} is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1;
   wherein the wavy line of M′ indicates the point of covalent attachment to the remainder of the structure of L′ and the wavy line of Formula (iii) indicates the point of covalent attachment to the degrader compound (D); and
   wherein subscript dd, U, X, YY, NN, EE, AA, J, and G are as defined herein for Antibody Drug Conjugate compounds.

In some embodiments, L′ is a linker precursor of Formula (iii):

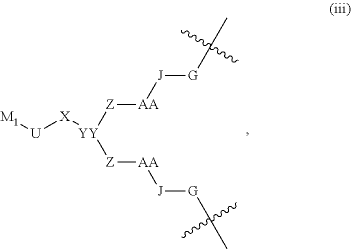

wherein
M′ is selected from the group consisting of:

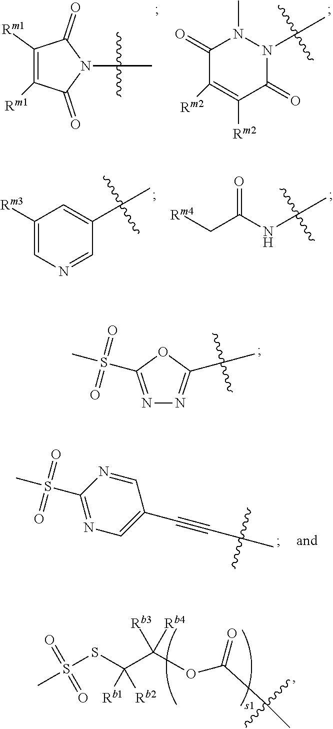

wherein
   each R^{m1} and R^{m2} is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1;
   wherein the wavy line of M′ indicates the point of covalent attachment to the remainder of the structure of L′ and the wavy line of Formula (iii) indicates the point of covalent attachment to the degrader compound (D); and
   wherein U, X, YY, ZZ, AA, J, and G are as defined herein for Antibody Drug Conjugate compounds.

In some embodiments, M′ is

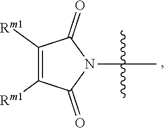

and each R^{m1} is independently hydrogen, halogen, or —S-Ph. In some embodiments, M′ is

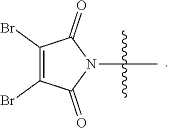 [SMILES: *N1C(=O)C(Br)=C(Br)C1=O]

In some embodiments, M′ is

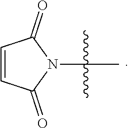 [SMILES: *N1C(=O)C=CC1=O]

In some embodiments, M′ is

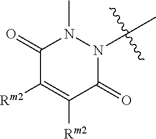

and each R^{m2} is hydrogen, halogen, or —S-Ph. In some embodiments, M′ is Br

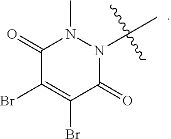 [SMILES: *n1c(=O)c(Br)c(Br)c(=O)n1C]

In some embodiments, M′ is

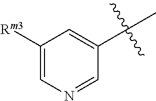

and R^{m3} is hydrogen, halogen, or —S-Ph. In some embodiments, M′ is

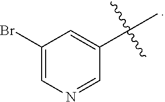 [SMILES: *c1cncc(Br)c1]

In some embodiments, M′ is

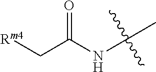

and R^{m4} is halogen. In some embodiments, M′ is

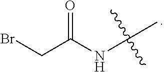 [SMILES: *NC(=O)CBr]

In some embodiments, M′ is H

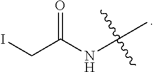 [SMILES: *NC(=O)CI]

In some embodiments, the M′ moiety of the linker precursor of the drug-linker compounds described herein comprises a reactive group that is capable of forming a covalent bond with an antibody, or antigen-binding fragment thereof, to form an Antibody Drug Conjugate compound as described herein. In some embodiments, the conjugating group is capable of forming a covalent bond with a cysteine residue of the antibody, or antigen-binding fragment thereof. In some embodiments, the conjugating group is capable of forming a covalent bond with a lysine residue of the antibody, or antigen-binding fragment thereof.

In some embodiments, the Degrader-Linker compound is represented by the structure of:

L′-D

wherein,
L′ is a linker precursor; and
D is a degrader compound of Formula (I):

PTM-ULM (I),
wherein,
   PTM is a moiety of Formula IA:

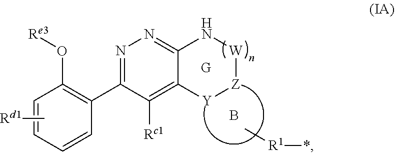

wherein,
   R¹ is a covalent bond, or chemical moiety that links PTM and ULM;
   is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z; and
ULM is a small molecule E3 Ubiquitin Ligase binding moiety that binds a Von Hippel-Lindau E3 Ubiquitin Ligase or a Cereblon E3 Ubiquitin Ligase.

In some embodiments, R¹ is a covalent bond that links PTM and ULM. In some embodiments, R¹ is a chemical moiety that links PTM and ULM. In some embodiments, n is 0. In some embodiments, n is 1. In some embodiments, each W is independently optionally substituted —CH₂—. In some embodiments, each W is independently optionally substituted —CH₂— or —C(O)—. In some embodiments, R^{c1} and R^{d1} are both H. In some embodiments, R^{e3} is hydrogen. In some embodiments, Z and Y are both N. In some embodiments, one of Y and Z is N, and the other is CR^{h} wherein R^{h}=H, C₁₋₃ alkyl, or absent. In some embodiments, R¹ is attached to Z, and Z is C and Y is N or CR^{h} wherein R^{h} is H or C₁₋₃ alkyl. In some embodiments, R¹ is attached to Y, and Y is C and Z is N or CR^{h} wherein R^{h} is H or C₁₋₃ alkyl. In some embodiments, B is an optionally substituted 5-7 membered cycloalkyl ring. In some embodiments, B is an optionally substituted 5-7 membered heteroaryl ring. In some embodiments, B is an optionally substituted 5-7 membered heterocyclic ring. In some embodiments, ULM binds a Von Hippel-Lindau E3 Ubiquitin Ligase. In some embodiments, ULM binds a Cereblon E3 Ubiquitin Ligase.

In some embodiments, the Degrader-Linker compound has the structure of:

L′-D

wherein,
D is a degrader compound of Formula (i):

PTM-ULM (i),
wherein,
   PTM is a moiety of Formula ia:

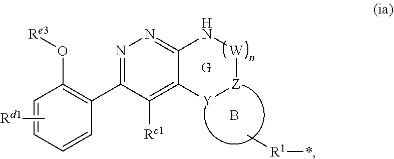

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L′ is a linker precursor of Formula (ii):

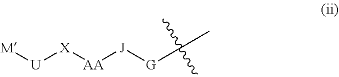

wherein
   M′ is selected from the group consisting of:

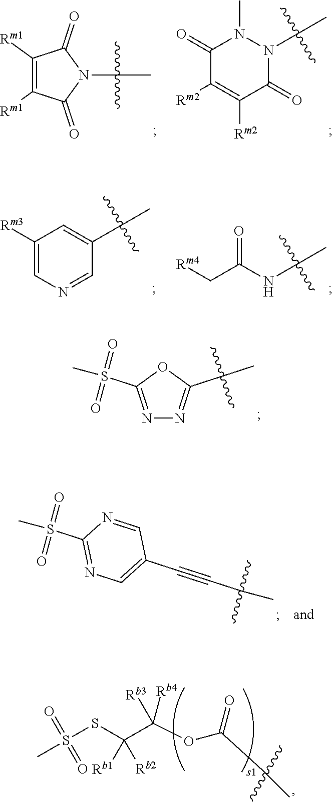

wherein
   each R^{m1} and R^{m2}is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1;
   wherein the wavy line indicates the point of covalent attachment to the remainder of the structure of L′;
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript I is an integer ranging from 0 to 6;
   —C(O)—C(R^{x5})(R^{x6})—, wherein R^{x5} is hydrogen or C₁-C₆ alkyl and R^{x6} is hydrogen, C₁-C₆ alkyl, or

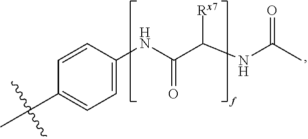

wherein subscript f is an integer ranging from 0 to 4 and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
AA is absent or has the structure of:

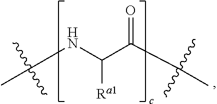

wherein subscript c is an integer ranging from 1 to 12; each R^{a1} is independently —COOH, —NH₂, or an independently selected side chain of an amino acid;
J is absent, —(R^{j3})N(C(R^{j1})(R^{j2}))ₘ₁—, or has the structure of:

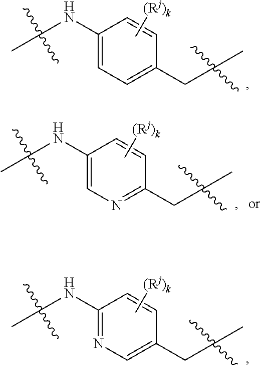

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
G is absent,

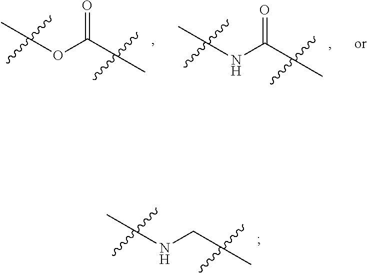

and
   wherein the wavy line to G in Formula (ii) indicates the point of covalent attachment to D.

In some embodiments, the wavy line to G of Formula (ii) indicates the point of covalent attachment to D. In some embodiments, the wavy line to G of Formula (ii) indicates the point of covalent attachment to a secondary alcohol group or a secondary amine group of D. In some embodiments, the secondary alcohol group is the alcohol group of a phenol group of D. In some embodiments, the secondary amine group is the imido nitrogen of a glutarimide group of D. In some embodiments, the secondary amine group is the amine nitrogen of a piperazine group of D.

In some embodiments, R¹ is a covalent bond that links PTM and ULM. In some embodiments, R¹ is a chemical moiety that links PTM and ULM. In some embodiments, n is 0. In some embodiments, n is 1. In some embodiments, each W is independently optionally substituted —CH₂— or —C(O)—. In some embodiments, R^{c1} and R^{d1} are both H. H. In some embodiments, R^{e3} is hydrogen. In some embodiments, Z and Y are both N. In some embodiments, one of Y and Z is N, and the other is CR^{h}wherein R^{h}=H, C₁₋₃ alkyl, or absent. In some embodiments, R¹ is attached to Z, and Z is C and Y is N or CR^{h} wherein R^{h} is H or C₁₋₃ alkyl. In some embodiments, R¹ is attached to Y, and Y is C and Z is N or CR^{h} wherein R^{h} is H or C₁₋₃ alkyl. In some embodiments, B is an optionally substituted 5-7 membered cycloalkyl ring. In some embodiments, B is an optionally substituted 5-7 membered heteroaryl ring. In some embodiments, B is an optionally substituted 5-7 membered heterocyclic ring. In some embodiments, ULM binds a Von Hippel-Lindau E3 Ubiquitin Ligase. In some embodiments, ULM binds a Cereblon E3 Ubiquitin Ligase.

In some embodiments, M′ is

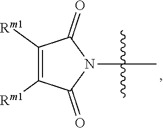

In some embodiments, U is absent. In some embodiments, U is —(CH₂)_{b}(R^{v1})ₛₛ(C═O)ᵤ(NH)ᵥ—. In some embodiments, X is absent. In some embodiments, X is —(CH₂CH₂O)_{w}CH₂CH₂C(O)—. In some embodiments, X is —CH(R^{x1})(CH₂)ₙC(O)—. In some embodiments, X is —C(O)—C(R^{x3})(R^{x4})—C(O)—. In some embodiments, X is -heterocycloalkyl-O(CH₂)ᵢC(O)—. In some embodiments, X is —C(O)—C(R^{x5})(R^{x6})—. In some embodiments, AA is absent. In some embodiments, AA has the structure of

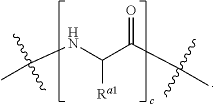

In some embodiments, J is absent. In some embodiments, J is —(R^{j3})N(C(R^{j1})(R^{j2}))ₘ—. In some embodiments, J has the structure of

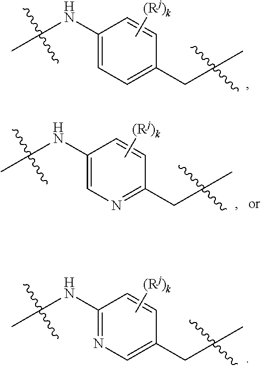

In some embodiments, J has the structure of

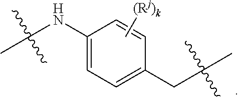

In some embodiments, J has the structure of

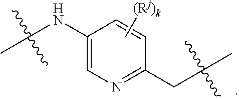

In some embodiments, J has the structure of

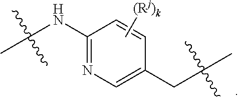

In some embodiments G is absent. In some embodiments, G is

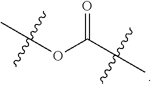 [SMILES: *OC(*)=O]

In some embodiments, G is

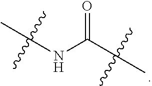 [SMILES: *NC(*)=O]

In some embodiments, G is

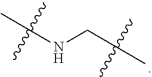 [SMILES: *CN*]

In some embodiments, R^{j} is a covalent bond. In some embodiments, R^{j} is a chemical moiety represented by the formula: -(A)_{q}-, wherein: q is an integer from 1 to 14; each A is independently selected from the group consisting of CR^{1a}R^{1b}, O, S, SO, SO₂, NR^{1c}SO₂NR^{1c}, SONR¹⁰, SO(═NR^{1c}), SO(═NR^{1c})NR^{1d}, CONR^{1c}, NR^{1c}CONR^{1d}, NR^{1c}C(O)O, NR^{1c}SO₂NR^{1d} CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b}groups, 3-11 membered heterocyclyl optionally substituted with 0-6 R^{1a} and/or R^{1b} groups, aryl optionally substituted with 0-6 R^{1a} and/or R^{1b} groups, and heteroaryl optionally substituted with 0-6 R^{1a} and/or R^{1b} groups, wherein R^{1a}, R^{1b}, R^{1c}, R^{1d} and R^{1e} are each independently, —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC1-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups. In some embodiments, q is 1. In some embodiments, q is 2. In some embodiments, q is 3. In some embodiments, q is 4. In some embodiments, q is an integer from 1 to 4. In some embodiments, q is 5. In some embodiments, q is 6. In some embodiments, q is 7. In some embodiments, q is 8. In some embodiments, q is an integer from 1 to 8. In some embodiments, q is 10. In some embodiments, q is 12. In some embodiments, q is 14.

In some embodiments, ULM binds Von Hippel-Lindau E3 Ubiquitin Ligase. In some embodiments, ULM is a moiety having the Formula ULM-I-VHL-1:

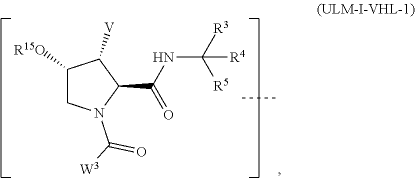

wherein
the dashed line ( [symbol] ) indicates the position of attachment of ULM-I-VHL-1 to R¹;
R¹⁵ is hydrogen or —PO₃H₂;
V is H or F;
R³ is optionally substituted phenyl, optionally substituted napthyl, or an optionally substituted 5-10 membered heteroaryl;
one of R⁴ or R⁵ is H, deuterium, haloalkyl, optionally substituted alkyl, optionally substituted cycloalkyl, optionally substituted heterocycloalkyl, —COR^{dv}, or CONR^{e1}R^{e2};
the other of R⁴ or R⁵ is H or deuterium;
or R⁴ and R⁵, together with the carbon atom to which they are both attached, form an optionally substituted 3-5 membered cycloalkyl or heterocyclyl;
W³ is an optionally substituted aryl, optionally substituted heteroaryl, or

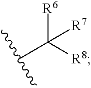

R⁶ and R⁷ are independently H, deuterium, optionally substituted alkyl, optionally substituted cycloalkyl, or optionally substituted haloalkyl,
or R⁶, R⁷, and the carbon atom to which they are attached form an optionally substituted cycloalkyl or optionally substituted heterocyclyl;
R⁸ is an optionally substituted heterocyclyl, optionally substituted heteroaryl, optionally substituted aryl, CONR^{av}R^{bv}, NR^{av}R^{bv},

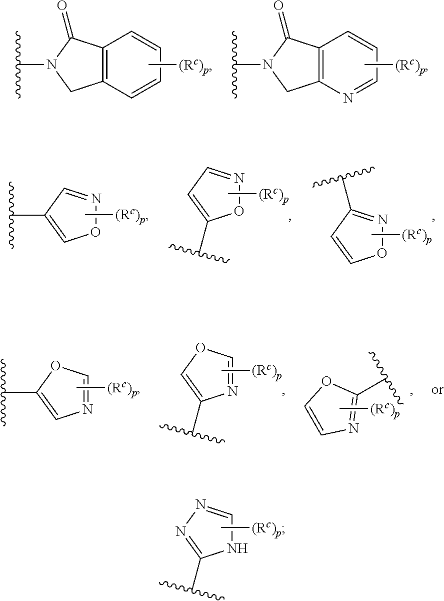

R^{av} is H or optionally substituted alkyl;
R^{bv} is H, —C(O)—* wherein * is a point of attachment to R¹, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{c} is independently H, halo, optionally substituted alkoxy, cyano, optionally substituted alkyl, haloalkyl, or haloalkoxy;
each R^{dv} is independently H, optionally substituted alkyl or NR^{e1}R^{e2};
each R^{e1} and R^{e2} is independently H, deuterium, or optionally substituted alkyl,
or R^{e1} and R^{e2} together with the nitrogen atom to which they are attached form a 4-7 membered heterocyclyl; and
p is 0, 1, 2, 3, or 4.

In some embodiments, ULM is a moiety having the Formula ULM-I:

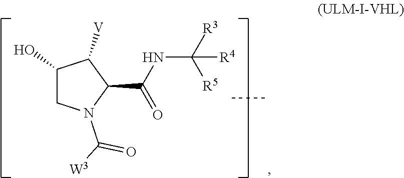

wherein
the dashed line ( [symbol] ) indicates the position of attachment of ULM-I-VHL to R¹;
V is H or F;
R³ is optionally substituted phenyl, optionally substituted napthyl, or an optionally substituted 5-10 membered heteroaryl;
one of R⁴ or R⁵ is H, deuterium, haloalkyl, optionally substituted alkyl, optionally substituted cycloalkyl, optionally substituted heterocycloalkyl, —COR^{dv}, or CON^{e1}R^{e2};
the other of R⁴ or R⁵ is H or deuterium;
or R⁴ and R⁵, together with the carbon atom to which they are both attached, form an optionally substituted 3-5 membered cycloalkyl or heterocyclyl;
W³ is an optionally substituted aryl, optionally substituted heteroaryl, or

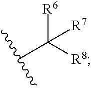

R⁶ and R⁷ are independently H, deuterium, optionally substituted alkyl, optionally substituted cycloalkyl, or optionally substituted haloalkyl,
or R⁶, R⁷, and the carbon atom to which they are attached form an optionally substituted cycloalkyl or optionally substituted heterocyclyl;
R⁸ is an optionally substituted heterocyclyl, optionally substituted heteroaryl, optionally substituted aryl, CONR^{av}R^{bv}, NR^{av}R^{bv},

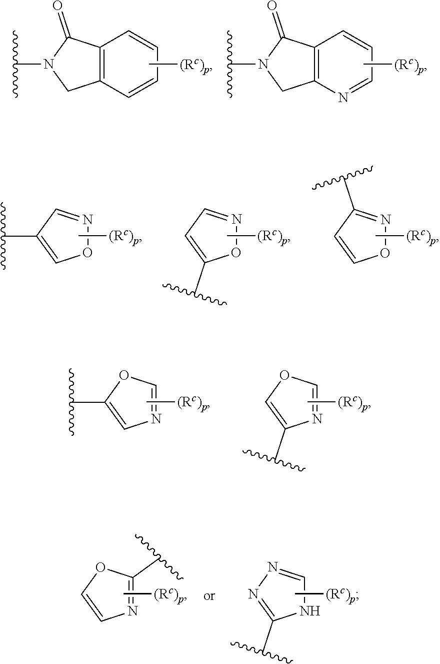

R^{av} is H or optionally substituted alkyl;
R^{bv} is H, —C(O)—* wherein * is a point of attachment to R¹, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{c} is independently H, halo, optionally substituted alkoxy, cyano, optionally substituted alkyl, haloalkyl, or haloalkoxy;
each R^{dv} is independently H, optionally substituted alkyl or NR^{e1}R^{e2};
each R^{e1} and R^{e2} is independently H, deuterium, or optionally substituted alkyl,
or R^{e1} and R^{e2} together with the nitrogen atom to which they are attached form a 4-7 membered heterocyclyl; and
p is 0, 1, 2, 3, or 4.

In some embodiments, V is H. In some embodiments, V is F. In some embodiments, R³ is optionally substituted phenyl. In some embodiments, R³ is optionally substituted napthyl. In some embodiments, R³ is an optionally substituted 5-10 membered heteroaryl. In some embodiments, one of R⁴ or R⁵ is H, deuterium, haloalkyl, optionally substituted alkyl, optionally substituted cycloalkyl, optionally substituted heterocycloalkyl, —COR^{dv}, or CONR^{e1}R^{e2}; and the other of R⁴ or R⁵ is H. In some embodiments, R⁴ and R⁵, together with the carbon atom to which they are both attached, form an optionally substituted 3-5 membered cycloalkyl or heterocyclyl. In some embodiments, W³ is an optionally substituted aryl. In some embodiments, W³ is an optionally substituted heteroaryl. In some embodiments, W³ is

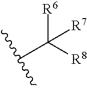

wherein R⁶ and R⁷ are independently H, deuterium, optionally substituted alkyl, optionally substituted cycloalkyl, or optionally substituted haloalkyl. In some embodiments, W³ is

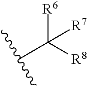

wherein R⁶, R⁷, and the carbon atom to which they are attached form an optionally substituted cycloalkyl or optionally substituted heterocyclyl. In some embodiments, R⁸ is an optionally substituted heterocyclyl, optionally substituted heteroaryl, optionally substituted aryl, CONR^{av}R^{bv}, or NR^{av}R^{bv}. In some embodiments, R⁸ is

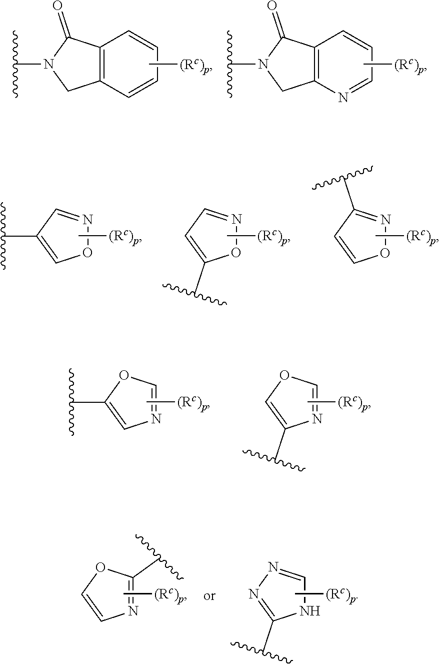

In some embodiments, ULM-I-VHL is a compound of formula:

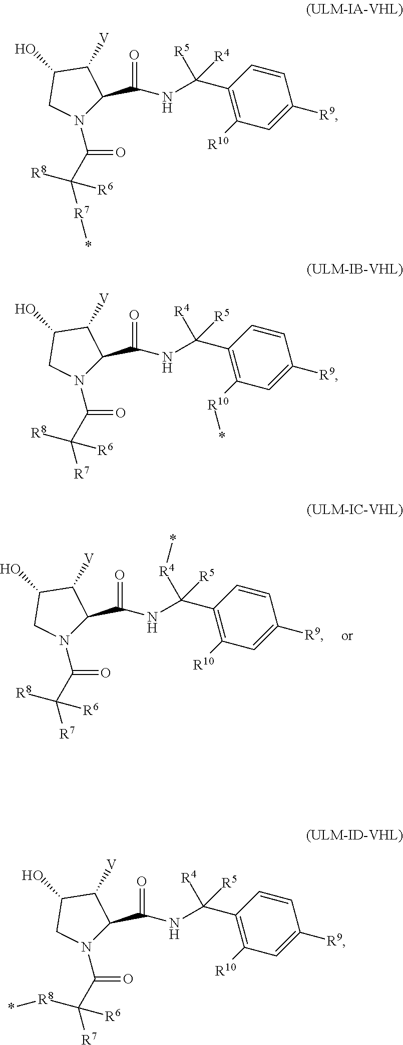

wherein * is a point of attachment of the ULM to R¹.

In some embodiments, ULM-I-VHL is a compound of formula (ULM-IA-VHL). In some embodiments, ULM-I-VHL is a compound of formula (ULM-IB-VHL). In some embodiments, ULM-I-VHL is a compound of formula (ULM-IC-VHL). In some embodiments, ULM-I-VHL is a compound of formula (ULM-ID-VHL).

In some embodiments, D has a structure of:

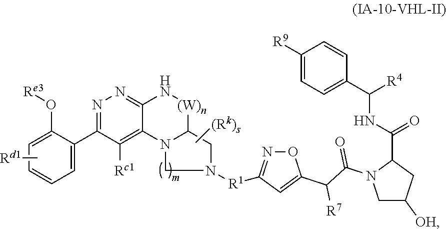

wherein
W is optionally substituted —CH₂—, —C(O)—, —S(O)—, or —S(O)₂—; wherein when n=2 or 3, only one W may be —C(O)—, —S(O)—, or —S(0)₂—;
n=0-3;
m=1-3;
R^{k}=H, deuterium, F, C₁₋₃ alkyl, C₁₋₃ haloalkyl, or C₁₋₄ alkoxyl;
s=0-3;
R^{c1} and R^{d1} are independently H, deuterium, Halo, C₁₋₃ alkyl, C₁₋₃ haloalkyl, or C₁₋₄ alkoxyl;
R^{e3} is H, —C(O)R, —CH₂—O—P(O)(OR^{g})₂, or —P(O)(OR^{g})₂; wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b}groups, —(CR^{1a}R^{1b})₁₋₅, —(CR^{1a}═CR^{1b})—(CR^{1a}R^{1b})₁₋₅-A- wherein A is O, S, or NR^{1c}—(CR^{1a}R^{1b})₁₋₅-A-(CR^{1a}R^{1b})₁₋₅— wherein A is O, S, or NR^{1c}—(CR^{1a}R^{b})₁₋₅-A-(CR^{1a}R^{b})₁₋₅-A- wherein A is O, S, or NR^{1c}, —(CR^{1a}R^{1b})₁₋₅—(CR^{1a}=CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b} groups)-, -(3-11 membered cycloalkyl optionally substituted with 0-6 R^{1a} and/or R^{1b} groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b}groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-(3-11 membered heterocyclyl optionally substituted with 0-6 R^{1a} and/or R^{1b} groups)-A- wherein A is O, S, or NR^{1c}—(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b}groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}—(CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}, —(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b} groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}-(3-11 membered cycloalkyl optionally substituted with 0-6 R^{1a} and/or R^{1b} groups)-(CR^{1a}R^{1b})₁₋₅₋, or -(3-11 membered heterocyclyl optionally substituted with 0-6 R^{1a} and/or R^{1b} groups)-(CR^{1a}R^{1b})₁₋₅.; or R^{j} is -(3-11 membered heterocyclyl optionally substituted with 0-6 R^{1a} and/or R^{1b} groups)-(CO)—(CR^{1a}R^{1b})₁₋₅-A- wherein A is O, S, or NR^{1c}; -(3-11 membered heterocyclyl optionally substituted with 0-6 R^{1a} and/or R^{1b} groups)-(CO)-A-(CR^{1a}R^{1b})₁₋₅— wherein A is O, S, or NR^{1c}; -A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}; -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-A-(CR^{1a}R^{1b})₁₋₅-A- wherein each A is independently O, S, or NR^{1c}; -(heteroaryl optionally substituted with 0-4 R^{1a} and/or R^{1b} groups)-A-(CR^{1a}R^{1b})₁₋₅— wherein A is O, S, or NR^{1c}; -(3-11 membered heterocyclyl optionally substituted with 0-6 R^{1a} and/or R^{1b}groups)-(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)—(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or —(CO)-(3-11 membered cycloalkyl optionally substituted with 0-6 R^{1a} and/or R^{1b} groups)-(CR^{1a}R^{1b})₁₋₅-A- wherein A is O, S, or NR^{1c};
R⁴ is H, optionally substituted alkyl, optionally substituted C₁-C₆alkyl, or —CH₃;
R⁷ is optionally substituted alkyl, preferably optionally substituted C₁-C₆alkyl, and more preferably C₁-C₆alkyl; and
R⁹ is H, deuterium, halo, —CN, —OH, —NO₂, —NR^{e1}R^{e2}, —OR^{e1}, —CONR^{e1}R^{e2}, —NR^{e1}COR^{e2}, —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R^{1a}, R^{1b}, R^{1c}, and R^{1c} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC1-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups; and
each R^{e1} and R^{e2} is independently H, deuterium, or optionally substituted alkyl, or R^{e1}
and R^{e2} together with the nitrogen atom to which they are attached form a 4-7 membered heterocyclyl.

In some embodiments, D has a structure of:

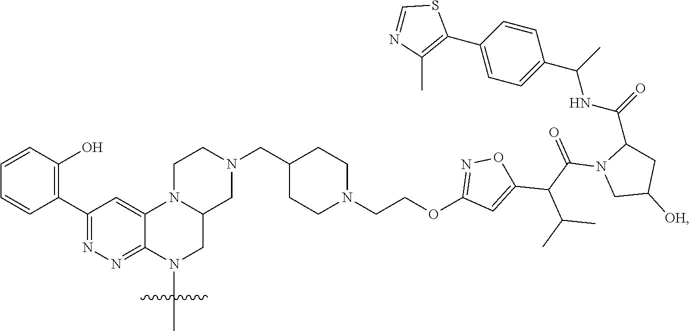 [SMILES: *N1CC2CN(CC3CCN(CCOc4cc(C(C(=O)N5CC(O)CC5C(=O)NC(C)c5ccc(-c6scnc6C)cc5)C(C)C)on4)CC3)CCN2c2cc(-c3ccccc3O)nnc21]

wherein the wavy line indicates the point of covalent attachment to L′.

In some embodiments, D has a structure of:

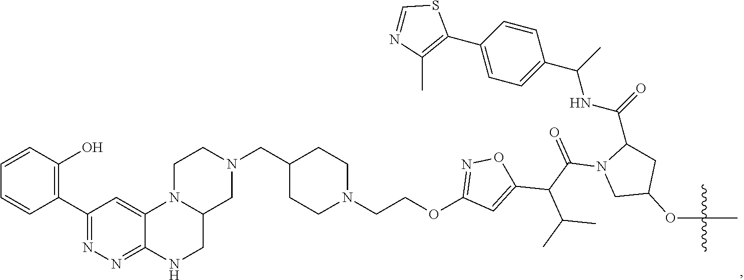 [SMILES: *OC1CC(C(=O)NC(C)c2ccc(-c3scnc3C)cc2)N(C(=O)C(c2cc(OCCN3CCC(CN4CCN5c6cc(-c7ccccc7O)nnc6NCC5C4)CC3)no2)C(C)C)C1]

wherein the wavy line indicates the point of covalent attachment to L′.

In some embodiments, D has a structure of:

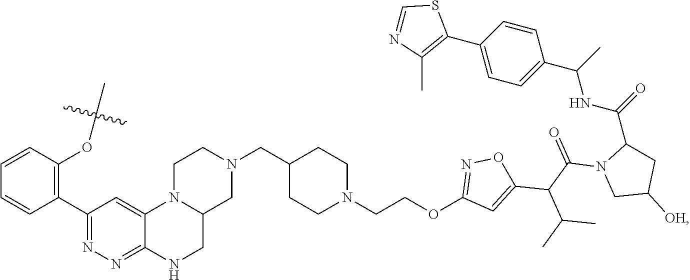 [SMILES: *Oc1ccccc1-c1cc2c(nn1)NCC1CN(CC3CCN(CCOc4cc(C(C(=O)N5CC(O)CC5C(=O)NC(C)c5ccc(-c6scnc6C)cc5)C(C)C)on4)CC3)CCN21]

wherein the wavy line indicates the point of covalent attachment to L′. In some embodiments, D has a structure of:

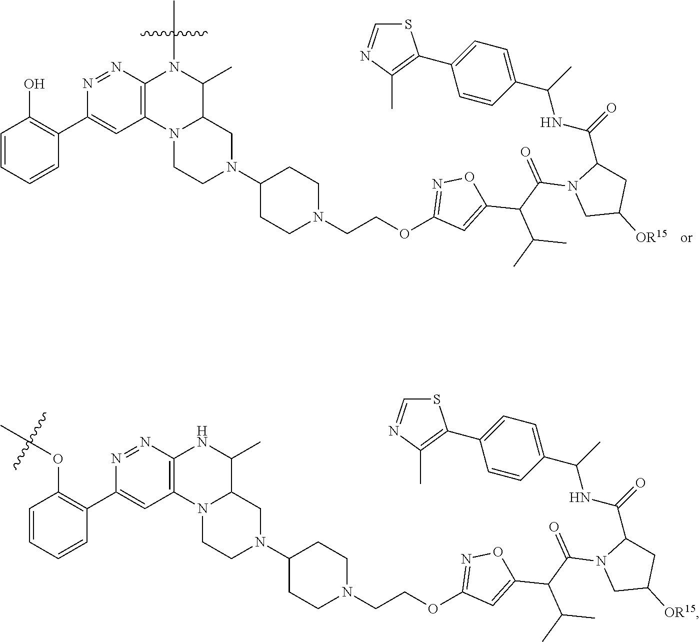

wherein R¹⁵is hydrogen or —PO₃H₂ and the wavy line indicates the point of covalent attachment to L′. In some embodiments, D has a structure of:

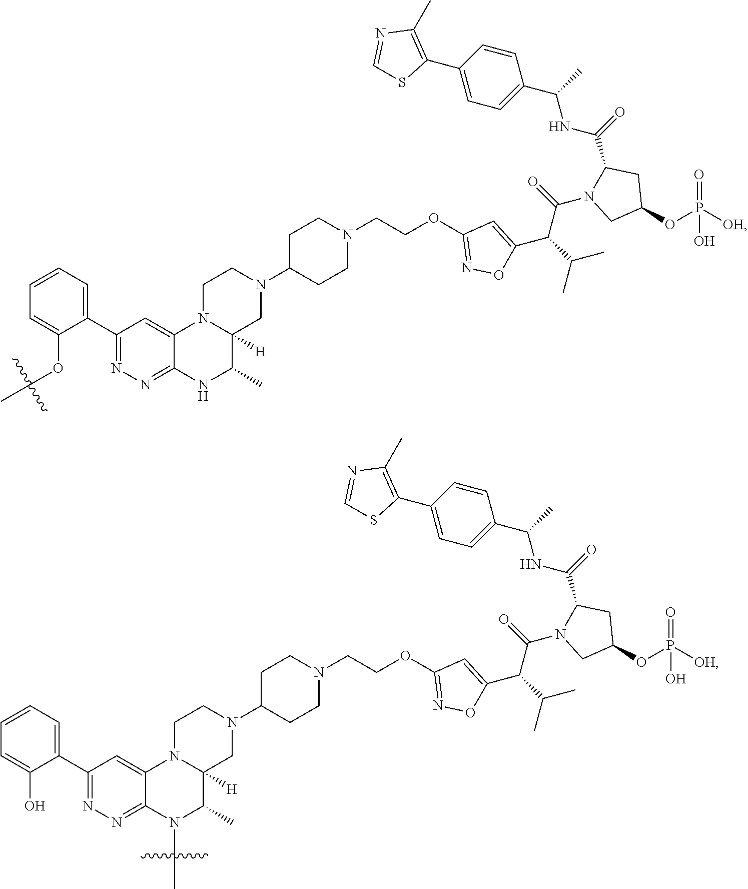 [SMILES: *Oc1ccccc1-c1cc2c(nn1)N[C@@H](C)[C@@H]1CN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)CCN21 ; *N1c2nnc(-c3ccccc3O)cc2N2CCN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)C[C@H]2[C@@H]1C]

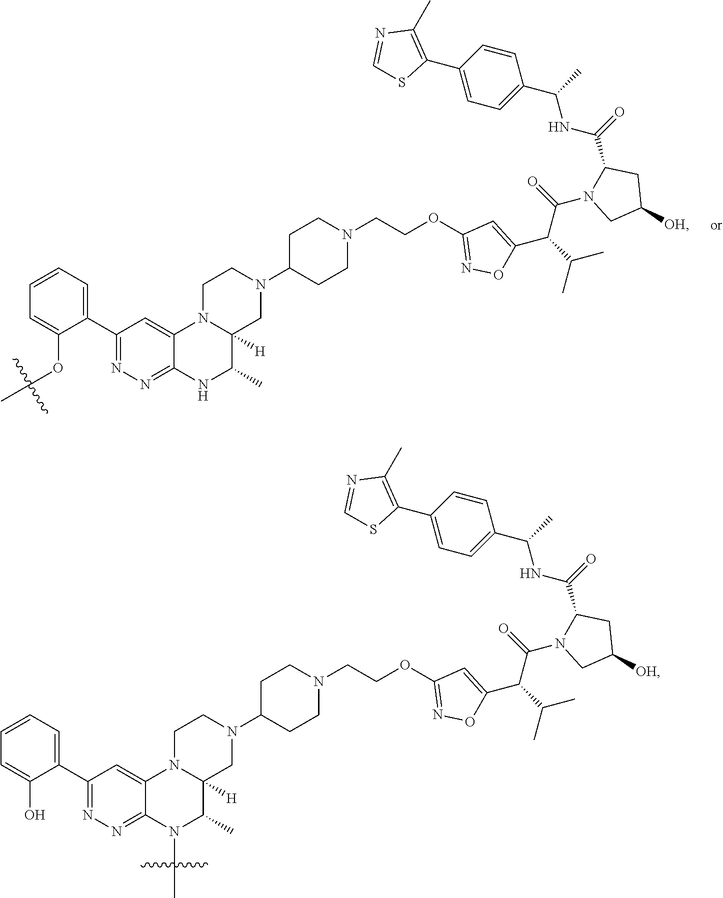 [SMILES: *Oc1ccccc1-c1cc2c(nn1)N[C@@H](C)[C@@H]1CN(C3CCN(CCOc4cc([C@H](C(=O)N5C[C@H](O)C[C@H]5C(=O)N[C@@H](C)c5ccc(-c6scnc6C)cc5)C(C)C)on4)CC3)CCN21 ; *N1c2nnc(-c3ccccc3O)cc2N2CCN(C3CCN(CCOc4cc([C@H](C(=O)N5C[C@H](O)C[C@H]5C(=O)N[C@@H](C)c5ccc(-c6scnc6C)cc5)C(C)C)on4)CC3)C[C@H]2[C@@H]1C]

wherein the wavy line indicates the point of covalent attachment to L′.

In some embodiments, ULM binds Cereblon E3 Ubiquitin Ligase. In some embodiments, ULM is a moiety having the Formula ULM-II-CRBN:

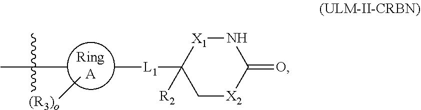

wherein
[symbol] is a point of attachment to PTM;
Ring A is a monocyclic, bicyclic or tricyclic aryl, heteroaryl or heterocycle group,
L¹ is a bond, —O—, —S—, —NR^{a}—, —C(R^{a})₂—, or —C(O)NR^{a}—;
X₁ is a bond, —C(O)—, —C(S)—, —CH₂—, —CHCF₃—, SO₂—, —S(O), P(O)R^{b}—or —P(O)OR^{b}—;
X₂ is —C(R^{a})₂—, —NR^{a}— or —S—;
R₂ is H, deuterium, optionally substituted C₁₋₄ alkyl, C₁₋₄ alkoxyl, C₁₋₄haloalkyl, —CN, —OR^{a}, —OR^{b} or —SR^{b};
each R₃ is independently H, deuterium, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{e2}R^{d} —NR^{a}R^{c2}, —C(O)R^{b}, —OC(O)R^{a}, —C(O)OR^{a}, —C(O)NR^{e2}R^{d}, —S(O)R^{b}, —S(O)₂NR^{e2}R^{d}, —S(O)(═NR^{b})R^{b}, —SF₅, —P(O)R^{b}R^{b}, —P(O)(OR^{b})(OR^{b}), —B(OR^{d})(OR^{c2}) or —S(O)₂R^{b};
each R^{a} is independently H, deuterium, —C(O)R^{b}, —C(O)OR^{c2}, —C(O)NR^{e2}R^{d}, —C(═NR^{b})NR^{b}R^{c2}, —C(═NOR^{b})NR^{b}R^{c2}, —C(═NCN)NR^{b}R^{c2}, —P(OR^{c2})₂, —P(O)R^{e2}R^{b}, —P(O)OR^{e2}OR^{b}, —S(O)R^{b}, —S(O)NR^{e2}R^{d}, —S(O)₂R^{b}, —S(O)₂NR^{e2}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, deuterium, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c2} or R^{d} is independently H, deuterium, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c2} and R^{d}, together with the atom to which they are both attached, form a monocyclic or multicyclic heterocycloalkyl, or a monocyclic or multicyclic heterocyclo-alkenyl group; and
o is 1, 2, 3, 4, or 5.

In some embodiments, ULM-II-CRBN is a compound of formula:

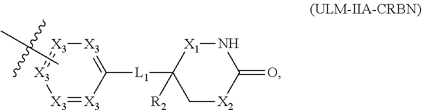

wherein each X₃ is independently N, N-oxide or CR₃ and at least one X₃ is N or N-oxide;
wherein [symbol] is a point of attachment to PTM; or

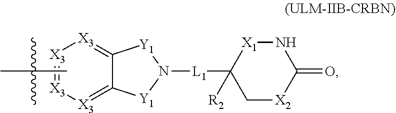

wherein each X₃ is independently N, N-oxide or CR₃;
wherein each Y₁ is independently —C(O)— or —C(R^{a})₂— and at least one Y₁ is —C(O)—; and
wherein [symbol] is a point of attachment to PTM; or

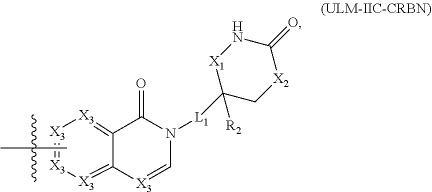

wherein each X₃ is independently N, N-oxide or CR₃ and wherein [symbol] is a point of attachment to PTM; or

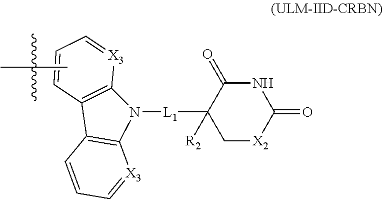

wherein each X₃ is independently N, N-oxide or CR₃ and wherein [symbol] is a point of [symbol] attachment to PTM.

In some embodiments, ULM-II-CRBN is a compound of formula (ULM-IIA-CRBN). In some embodiments, ULM-II-CRBN is a compound of formula (ULM-IIB—CRBN). In some embodiments, ULM-II-CRBN is a compound of formula (ULM-IIC—CRBN). In some embodiments, ULM-II-CRBN is a compound of formula (ULM-IID-CRBN).

In some embodiments, D has a structure of:

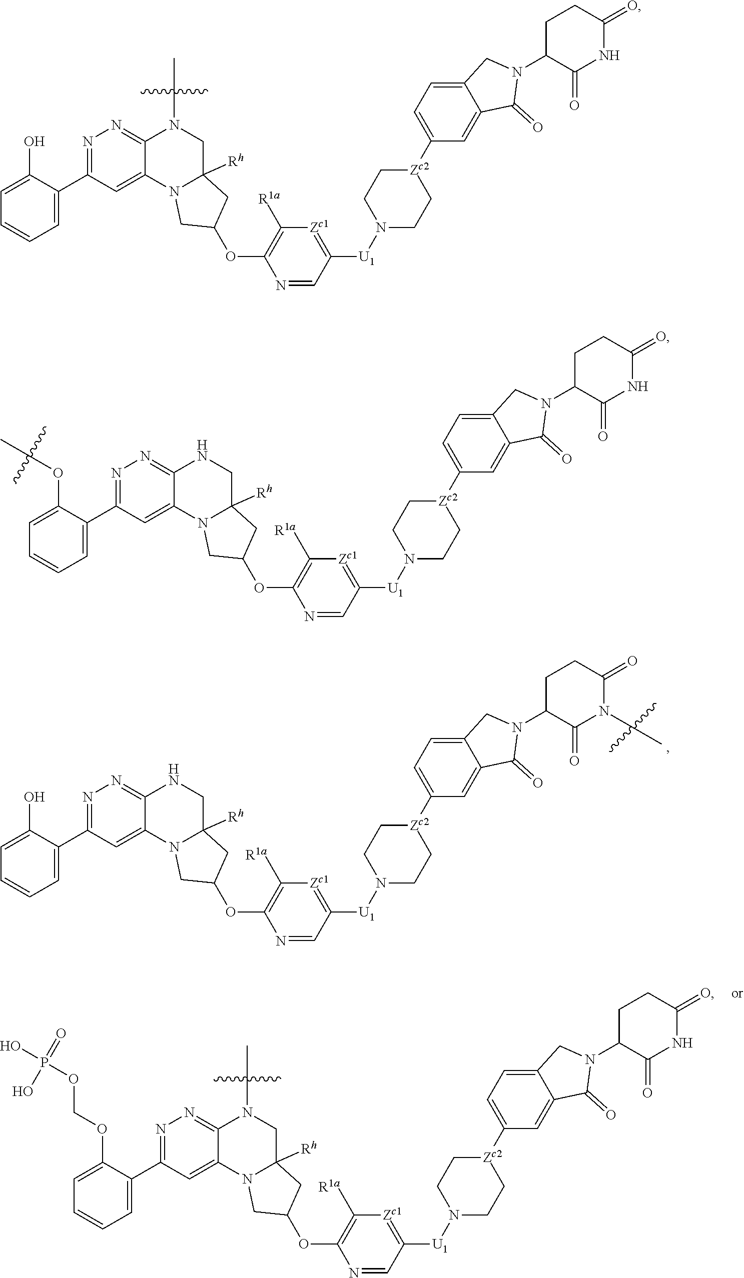

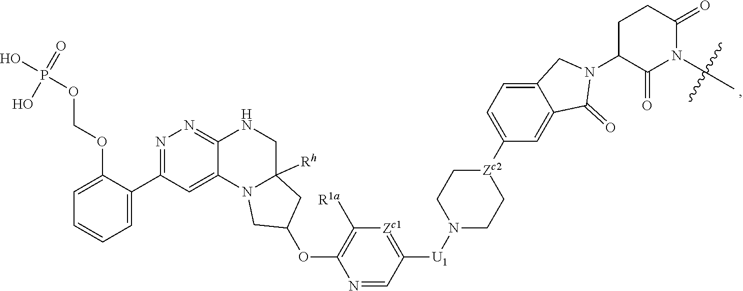

wherein
the wavy line indicates the point of covalent attachment to L′;
   R^{h} is methyl or ethyl;
   R^{1a} is hydrogen, C₁₋₃ alkyl, or halo;
   each of Z^{c1} and Z^{c2} is independently CH or N; and
U₁ is —CH₂— or —C(O)—.

In some embodiments, D has a structure of:

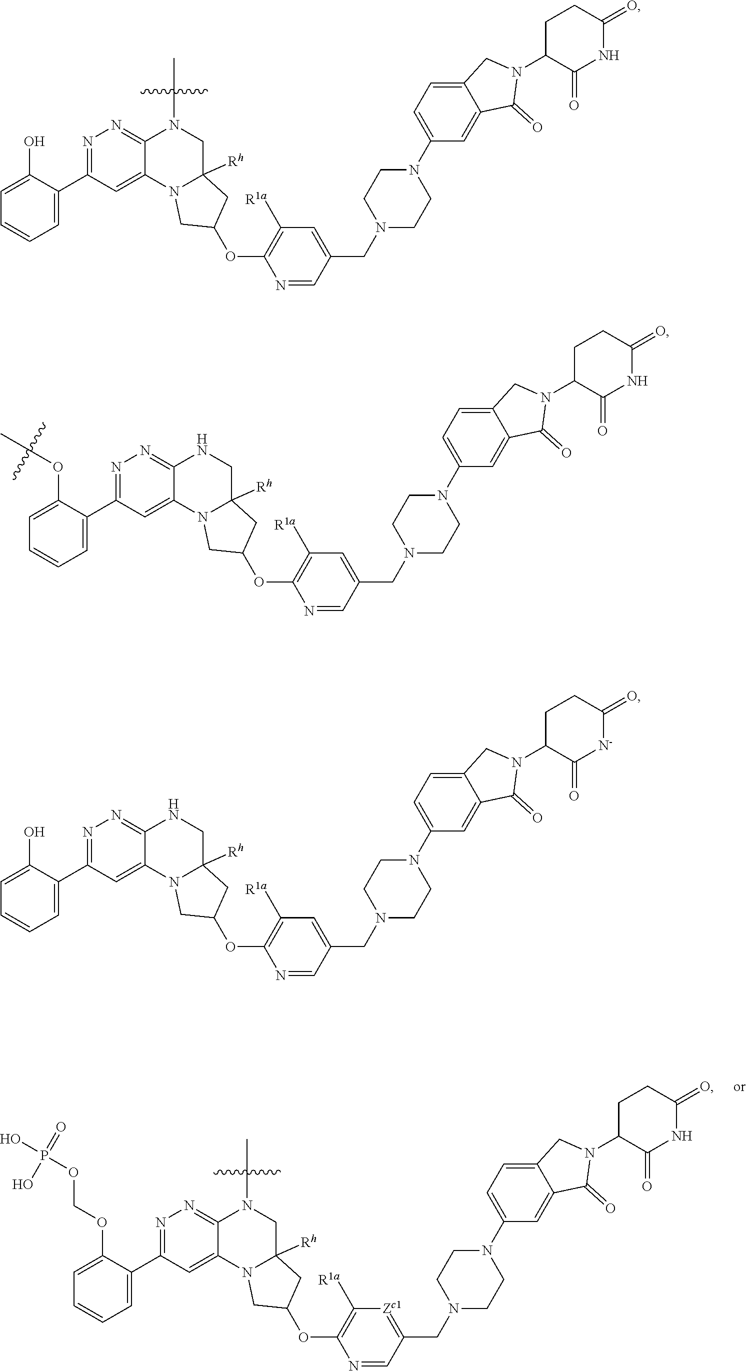

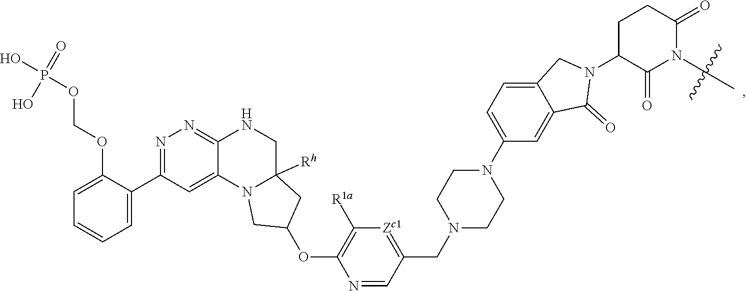

wherein R^{1a} is hydrogen, methyl, or fluorine, and the wavy line indicates the point of covalent attachment to L′.

In some embodiments, D has a structure of:

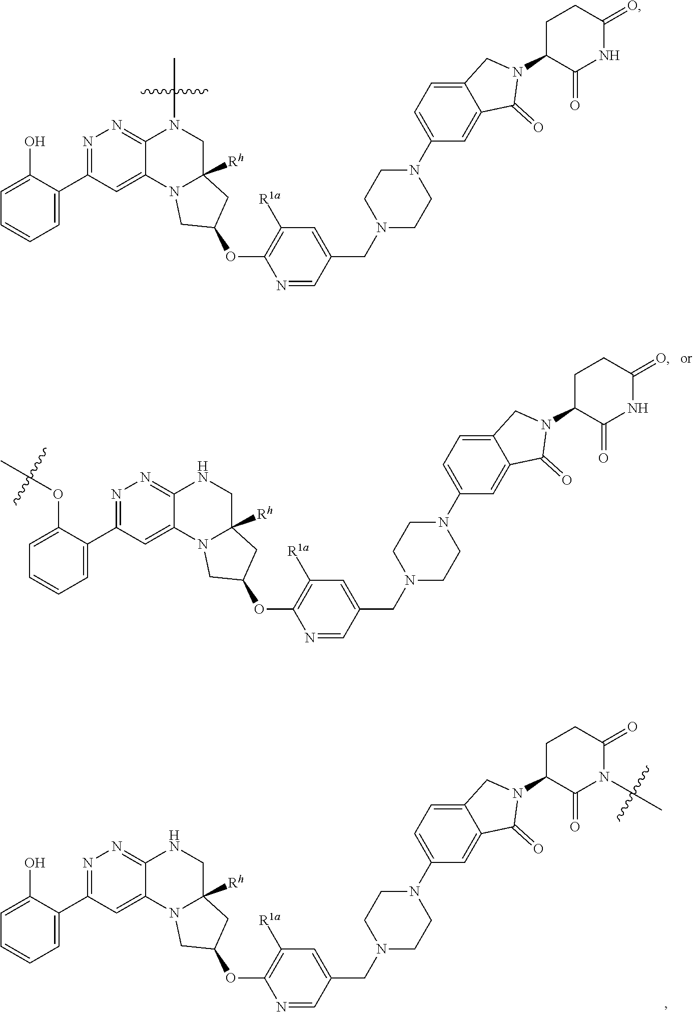

wherein R^{1a} is hydrogen, methyl, or fluorine, and the wavy line indicates the point of covalent attachment to L′.

In some embodiments, the degrader compound of Formula (I) has the structure of:

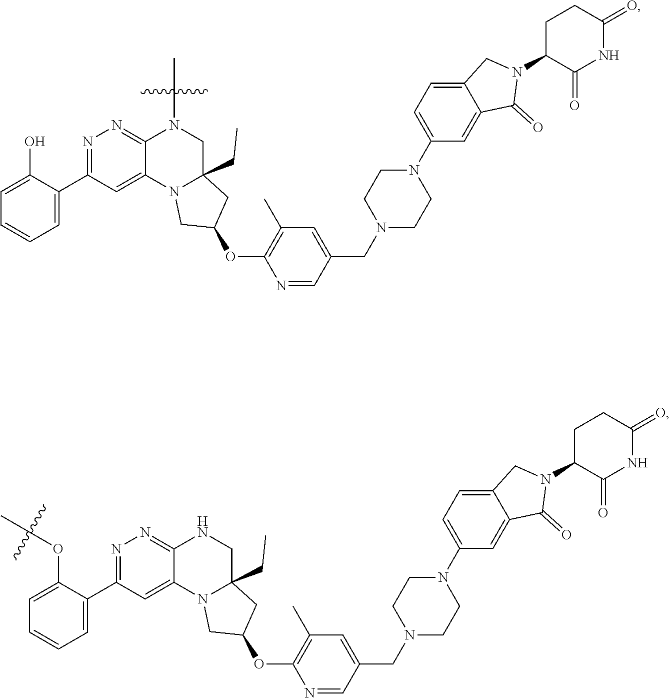 [SMILES: *N1C[C@]2(CC)C[C@@H](Oc3ncc(CN4CCN(c5ccc6c(c5)C(=O)N([C@H]5CCC(=O)NC5=O)C6)CC4)cc3C)CN2c2cc(-c3ccccc3O)nnc21 ; *Oc1ccccc1-c1cc2c(nn1)NC[C@]1(CC)C[C@@H](Oc3ncc(CN4CCN(c5ccc6c(c5)C(=O)N([C@H]5CCC(=O)NC5=O)C6)CC4)cc3C)CN21]

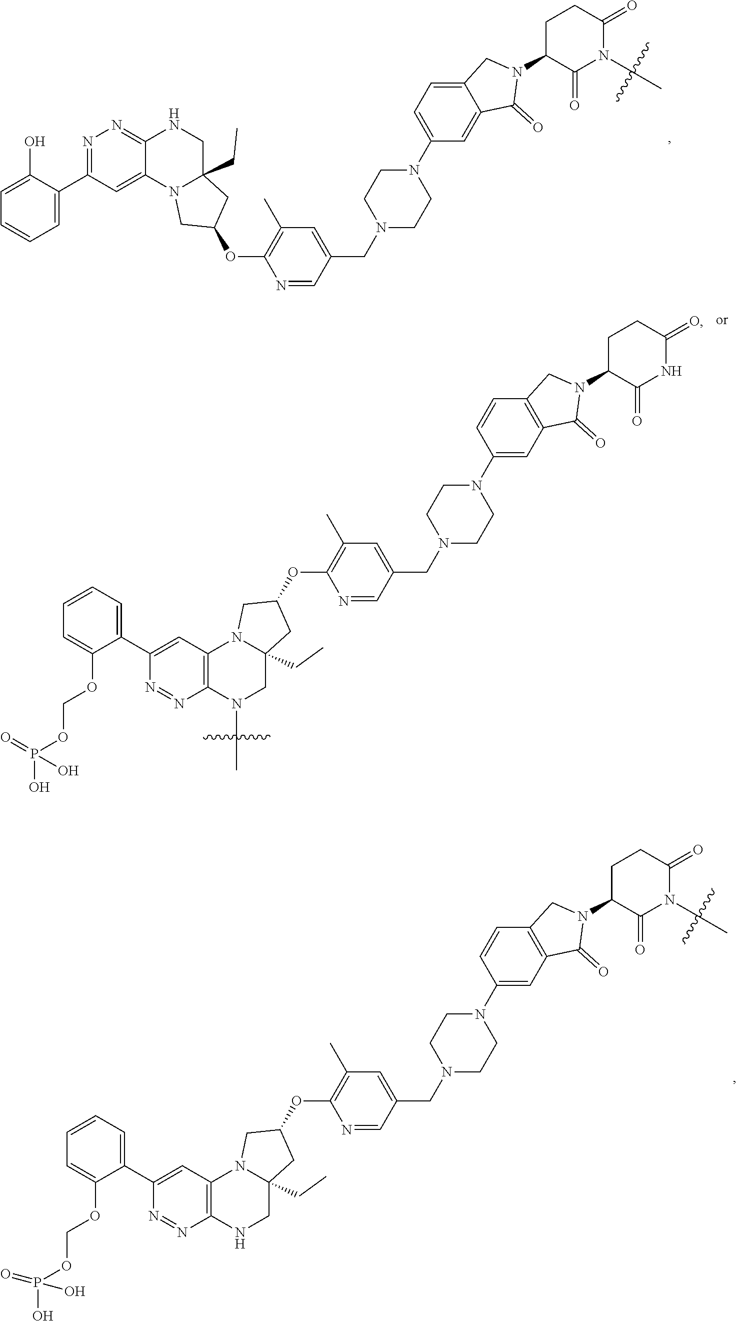 [SMILES: *N1C(=O)CC[C@H](N2Cc3ccc(N4CCN(Cc5cnc(O[C@H]6CN7c8cc(-c9ccccc9O)nnc8NC[C@]7(CC)C6)c(C)c5)CC4)cc3C2=O)C1=O ; "," ; *N1C[C@]2(CC)C[C@@H](Oc3ncc(CN4CCN(c5ccc6c(c5)C(=O)N([C@H]5CCC(=O)NC5=O)C6)CC4)cc3C)CN2c2cc(-c3ccccc3OCOP(=O)(O)O)nnc21 ; "or" ; *N1C(=O)CC[C@H](N2Cc3ccc(N4CCN(Cc5cnc(O[C@H]6CN7c8cc(-c9ccccc9OCOP(=O)(O)O)nnc8NC[C@]7(CC)C6)c(C)c5)CC4)cc3C2=O)C1=O ; ","]

or a pharmaceutically acceptable salt or solvate thereof, wherein the wavy line indicates the point of attachment to L′.

In some embodiments, the degrader compound of Formula (I) has the structure of:

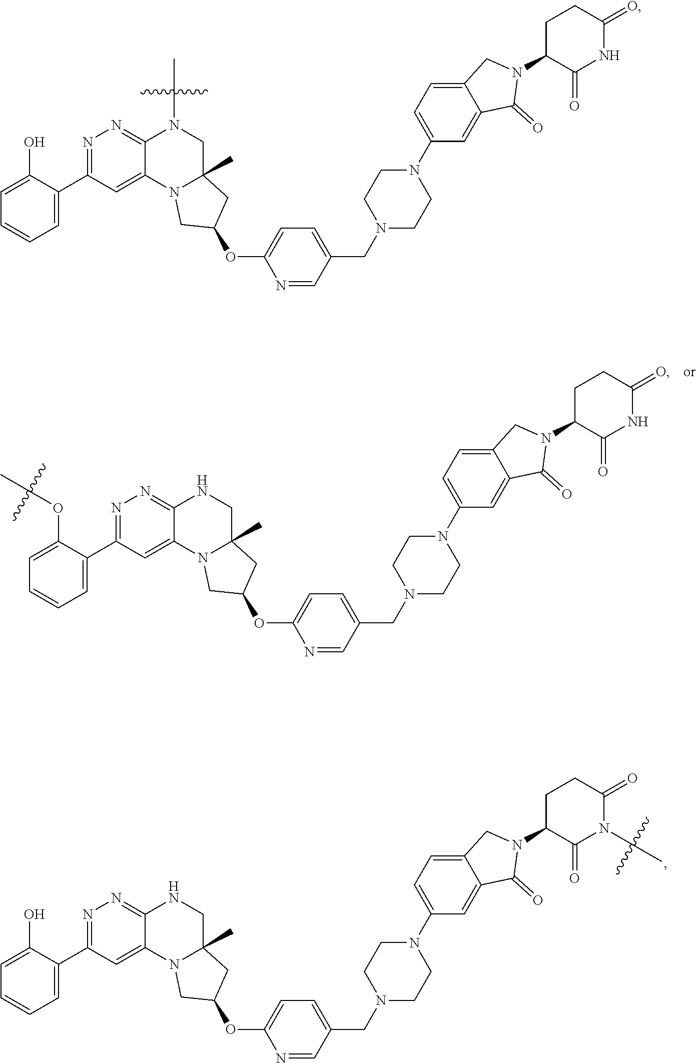 [SMILES: *N1C[C@]2(C)C[C@@H](Oc3ccc(CN4CCN(c5ccc6c(c5)C(=O)N([C@H]5CCC(=O)NC5=O)C6)CC4)cn3)CN2c2cc(-c3ccccc3O)nnc21 ; *Oc1ccccc1-c1cc2c(nn1)NC[C@]1(C)C[C@@H](Oc3ccc(CN4CCN(c5ccc6c(c5)C(=O)N([C@H]5CCC(=O)NC5=O)C6)CC4)cn3)CN21 ; *N1C(=O)CC[C@H](N2Cc3ccc(N4CCN(Cc5ccc(O[C@H]6CN7c8cc(-c9ccccc9O)nnc8NC[C@]7(C)C6)nc5)CC4)cc3C2=O)C1=O]

or a pharmaceutically acceptable salt or solvate thereof, wherein the wavy line indicates the point of attachment to L′.

In some embodiments, the Degrader-Linker compound has the formula of:

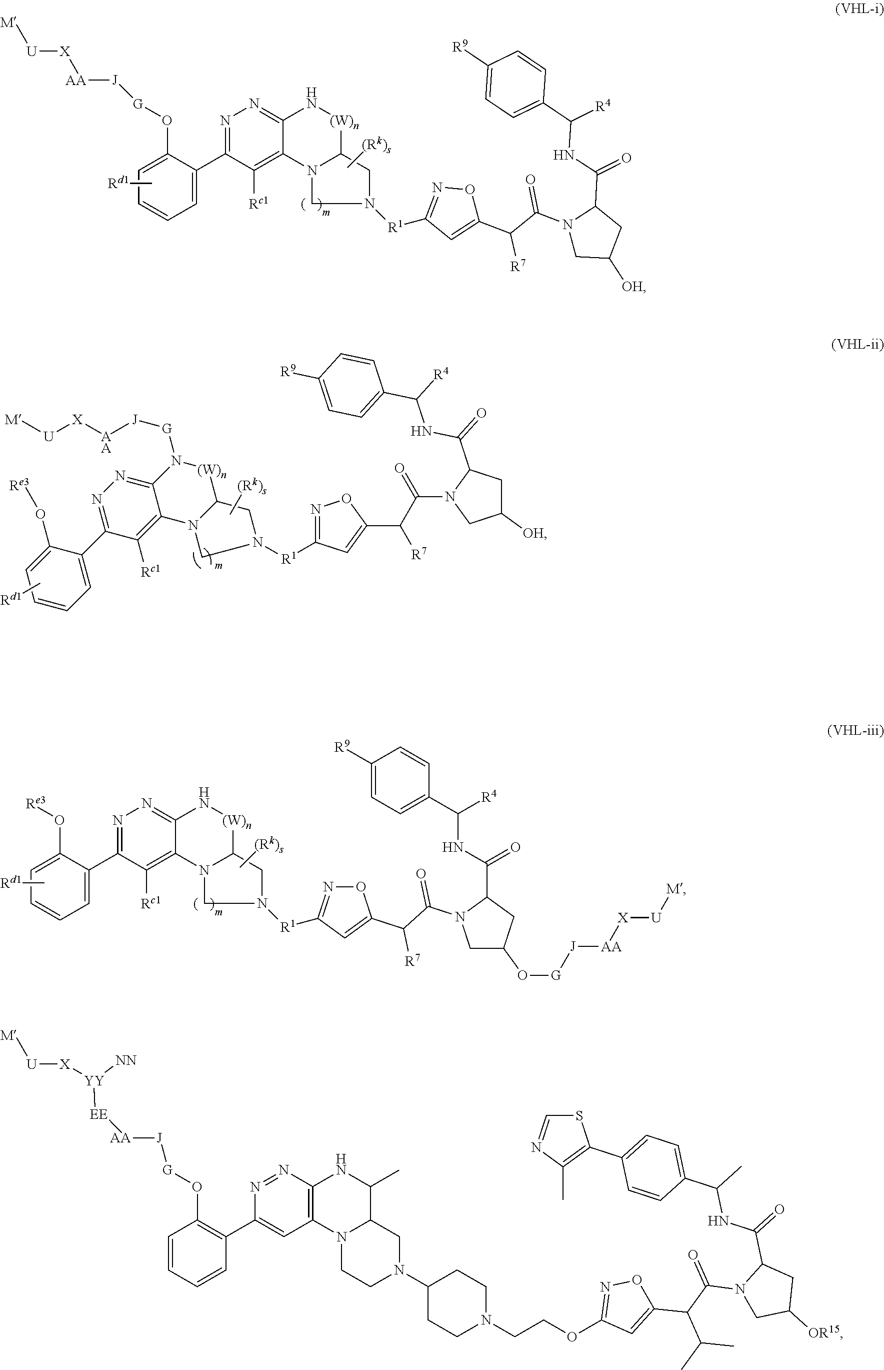

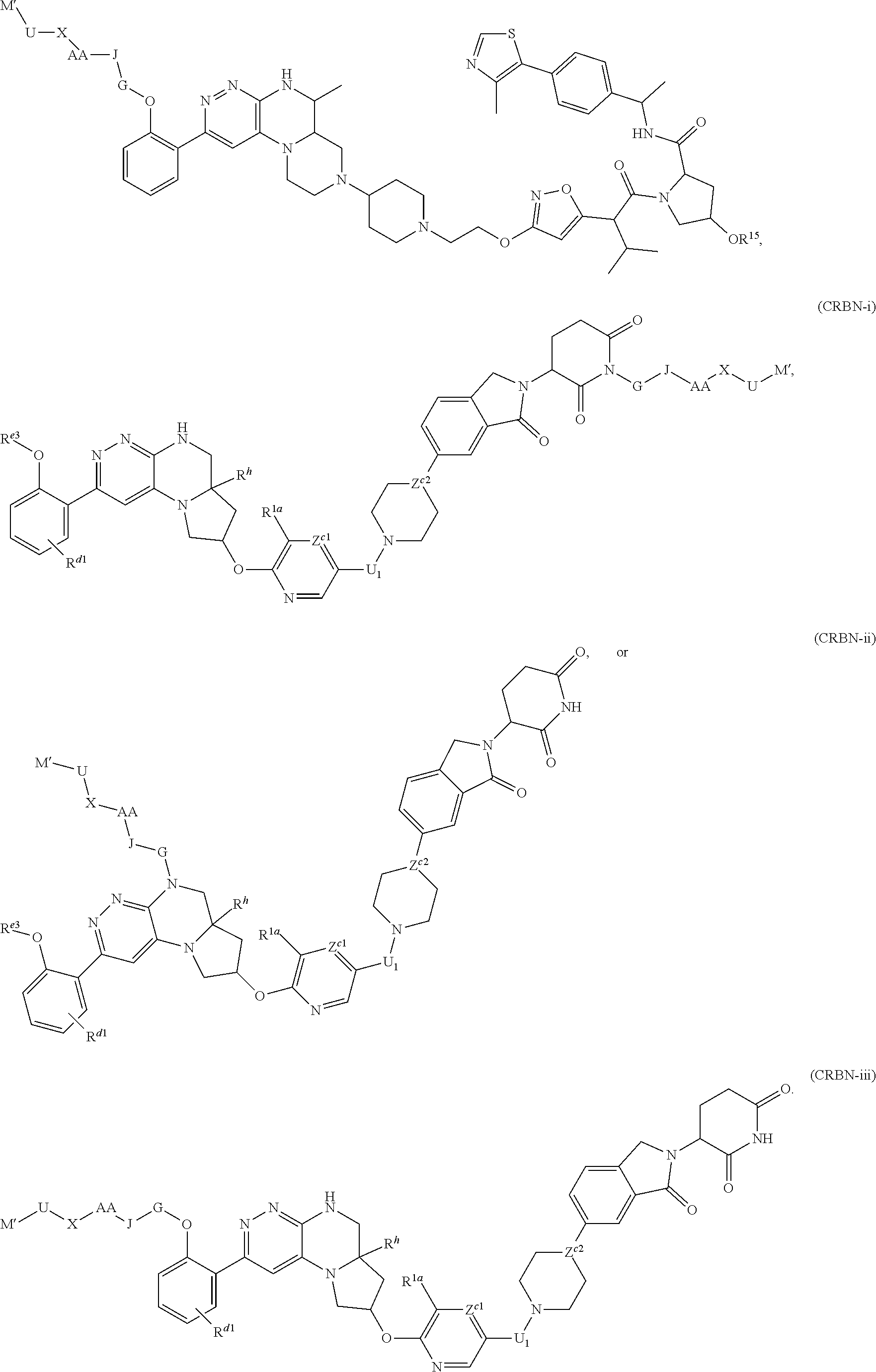

In some embodiments, the Degrader-Linker compound has the formula of (VHL-i). In some embodiments, the Degrader-Linker compound has the formula of (VHL-ii). In some embodiments, the Degrader-Linker compound has the formula of (VHL-iii). In some embodiments, the Degrader-Linker compound has the formula of (CRBN-i). In some embodiments, the Degrader-Linker compound has the formula of (CRBN-ii). In some embodiments, the Degrader-Linker compound has the formula of (CRBN-iii).

In some embodiments, AA is present; subscript c is an integer ranging between 1 and 12; and each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine.

In some embodiments, the Degrader-Linker compound is represented by the structure of

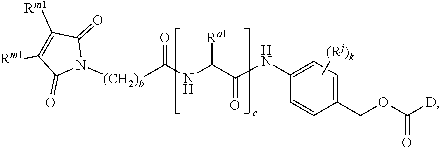

wherein each R^{m1} is hydrogen, halogen, or —S-Ph; subscript b is an integer ranging from 1 to 5; subscript c is an integer ranging from 1 to 5; each R^{a1} is independently the side chain of valine, glutamic acid, tryptophan, glycine, citrulline, lysine, alanine, or phenylalanine; each R³ is independently fluorine, chlorine, methyl, or —C(O)NHCH₃; and subscript k is an integer ranging from 0 to 4.

In some embodiments, the Degrader-Linker compound is represented by the structure of

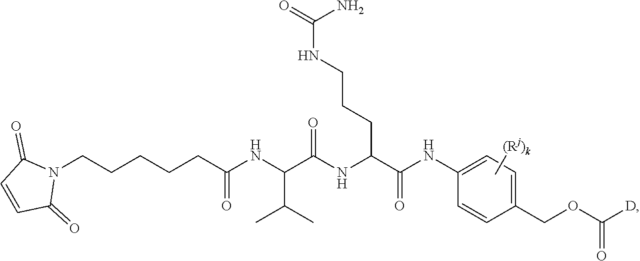

wherein each R^{j} is methyl, —F, —Cl, or —C(O)NHCH₃, and subscript k is 0 or 1. In some embodiments, the Degrader-Linker compound is represented by the structure of

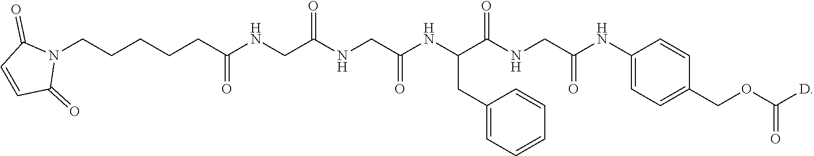

In some embodiments, the Degrader-Linker compound is represented by the structure of

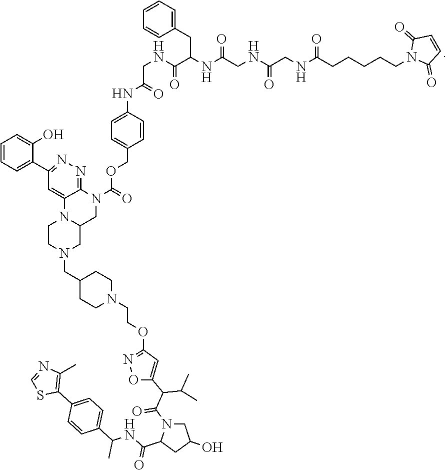 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6ccc(NC(=O)CNC(=O)C(Cc7ccccc7)NC(=O)CNC(=O)CNC(=O)CCCCCN7C(=O)C=CC7=O)cc6)CC5C4)CC3)no2)C(C)C)cc1]

In some embodiments, the Degrader-Linker compound is represented by the structure of

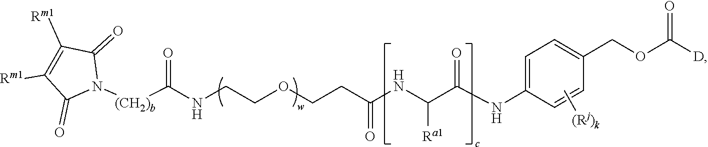

wherein each R^{m1} is hydrogen, halogen, or —S-Ph; subscript b is an integer ranging from 1 to 5; subscript w is an integer ranging from 1 to 8; subscript c is an integer ranging from 1 to 3; each R^{a1} is independently the side chain of valine, glutamic acid, tryptophan, glycine, citrulline, lysine, alanine, or phenylalanine; each R^{j} is independently fluorine, chlorine, methyl, or —C(O)NHCH₃; and subscript k is an integer ranging from 0 to 4.

In some embodiments, the Degrader-Linker compound is represented by the structure of

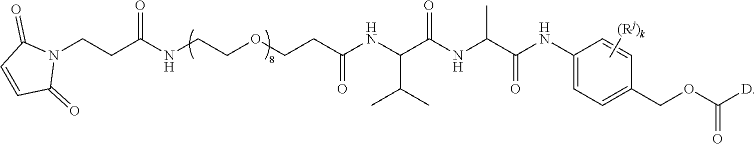

In some embodiments, the Degrader-Linker compound is represented by the structure of

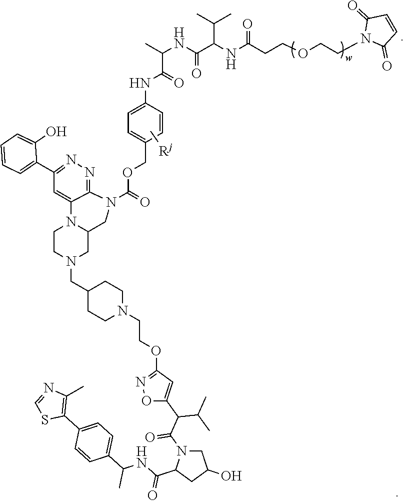

In some embodiments, the Degrader-Linker compound is represented by the structure of

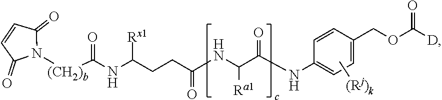

wherein subscript b is an integer ranging from 1 to 5; subscript c is an integer ranging from 1 to 3; R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; each R^{a1} is independently the side chain of valine, glutamic acid, tryptophan, glycine, citrulline, lysine, alanine, or phenylalanine; each R^{j} is independently fluorine, chlorine, methyl, or —C(O)NHCH₃; and subscript k is an integer ranging from 0 to 4.

In some embodiments, R^{x1} is

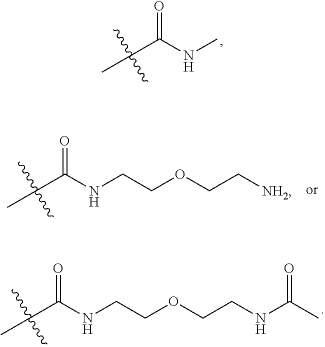

In some embodiments, R^{x1} is

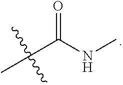 [SMILES: *C(=O)NC]

In some embodiments, R^{x1} is

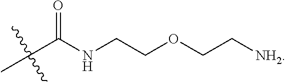 [SMILES: *C(=O)NCCOCCN]

In some
embodiments, R^{x1} is

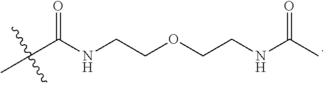 [SMILES: *C(=O)NCCOCCNC(C)=O]

In some embodiments, the Degrader-Linker compound is represented by the structure of

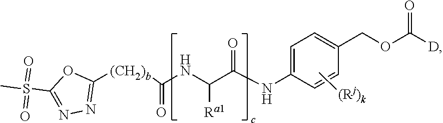

wherein subscript b is an integer ranging from 1 to 5; subscript c is 1 or 2; each R^{a1} is independently the side chain of valine or citrulline; each R³ is independently fluorine, chlorine, methyl, or —C(O)NHCH₃; and subscript k is an integer ranging from 0 to 4.

In some embodiments, the Degrader-Linker compound is represented by the structure of

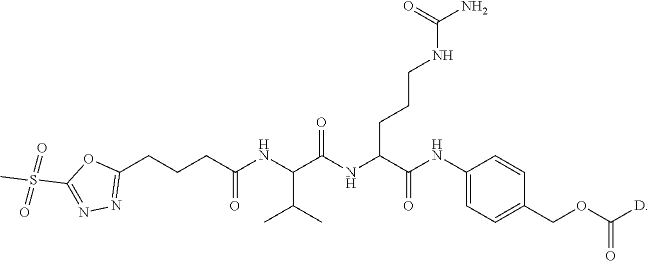

In some embodiments, the Degrader-Linker compound is represented by the structure of

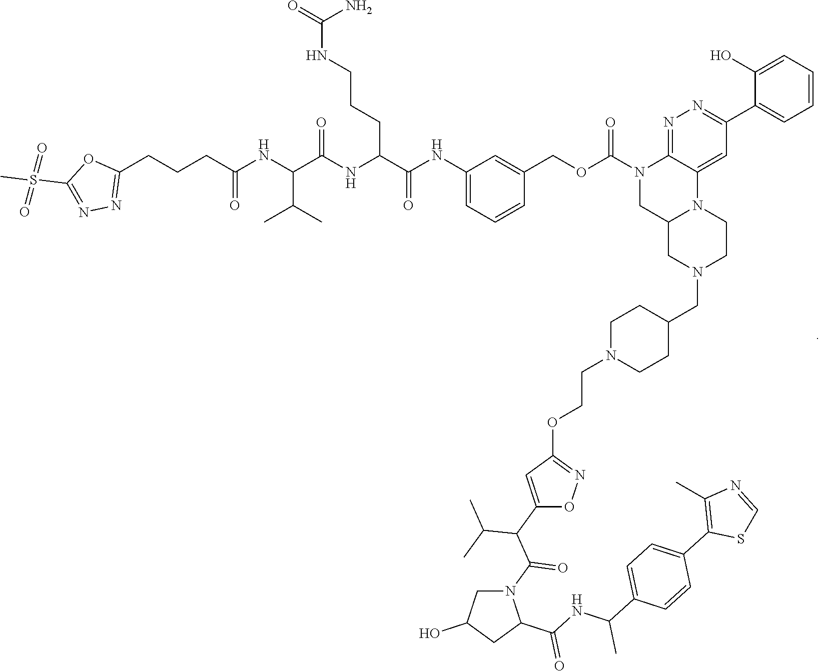 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6cccc(NC(=O)C(CCCNC(N)=O)NC(=O)C(NC(=O)CCCc7nnc(S(C)(=O)=O)o7)C(C)C)c6)CC5C4)CC3)no2)C(C)C)cc1]

In some embodiments, the Degrader-Linker compound is represented by the structure of

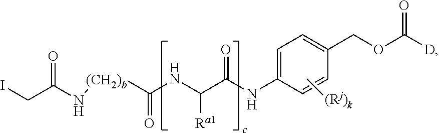

wherein subscript b is an integer ranging from 1 to 5; subscript c is 1 or 2; each R^{a1} is independently the side chain of valine or citrulline; each R^{j} is independently fluorine, chlorine, methyl, or —C(O)NHCH₃; and subscript k is an integer ranging from 0 to 4.

In some embodiments, the Degrader-Linker compound is represented by the structure of

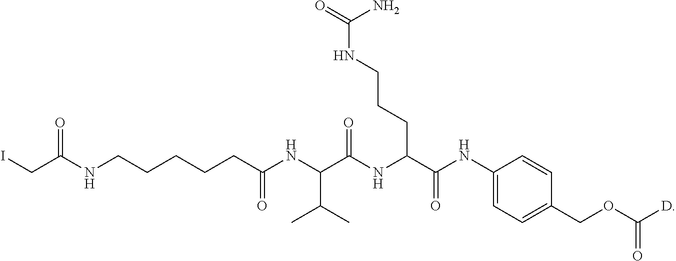

In some embodiments, the Degrader-Linker compound is represented by the structure of

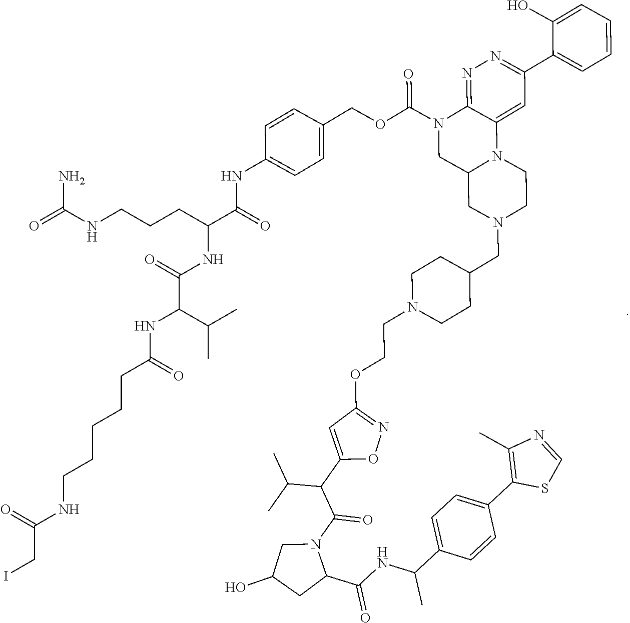 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6ccc(NC(=O)C(CCCNC(N)=O)NC(=O)C(NC(=O)CCCCCNC(=O)CI)C(C)C)cc6)CC5C4)CC3)no2)C(C)C)cc1]

In some embodiments, the Degrader-Linker compound is represented by the structure of

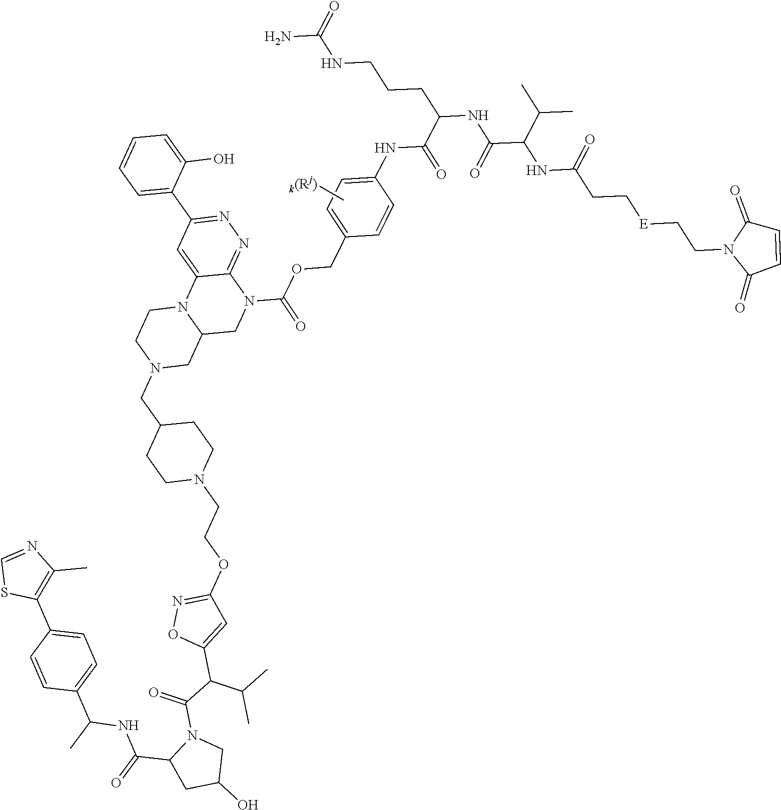

wherein E is —CH₂— or —O—; R^{j} is methyl, —F, —Cl, or —C(O)NHCH₃; and subscript k is 0 or 1. In some embodiments, E is —CH₂—. In some embodiments, E is —O—. In some embodiments, subscript k is 0. In some embodiments, subscript k is 1.

In some embodiments, the Degrader-Linker compound is represented by the structure of

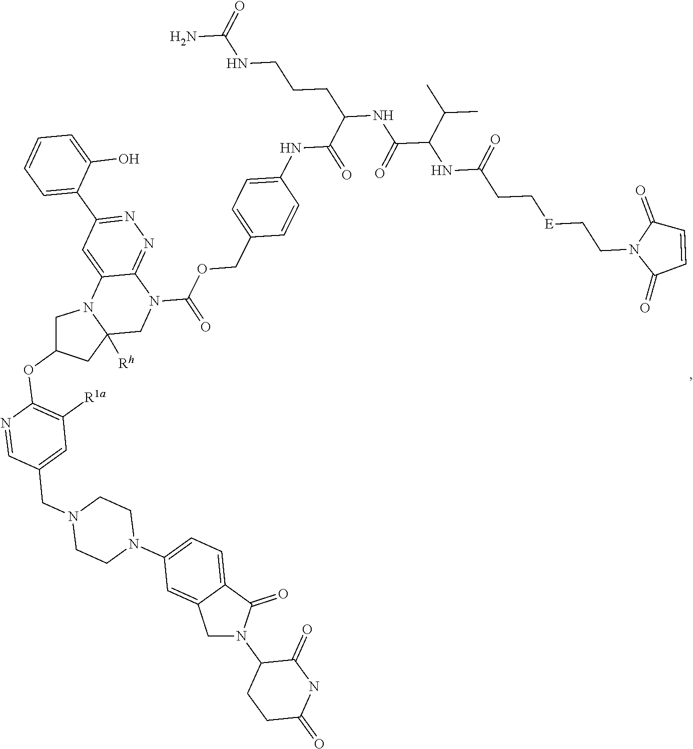

wherein E is —CH₂— or —O—; R^{h} is methyl or ethyl; and R^{1a} is H, C₁₋₃ alkyl, or halo. In some embodiments, R^{1a} is hydrogen or methyl. In some embodiments, E is —CH₂—. In some embodiments, E is-O—. In some embodiments, R^{h} is methyl. In some embodiments, R^{h} is ethyl. In some embodiments, R^{1a} is hydrogen. In some embodiments, R^{1a} is methyl.

In some embodiments, the Degrader-Linker compound is represented by the structure of

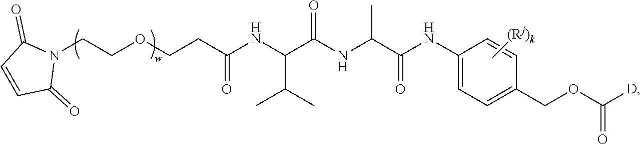

wherein subscript w is 1 or 2; each R^{j} is independently fluorine, chlorine, methyl, or —C(O)NHCH₃; and subscript k is an integer ranging from 0 to 4.

In some embodiments, the Degrader-Linker compound is represented by the structure of

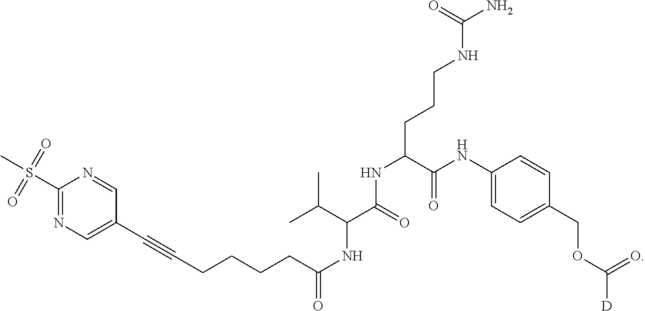 [SMILES: [2H]C(=O)OCc1ccc(NC(=O)C(CCCNC(N)=O)NC(=O)C(NC(=O)CCCCC#Cc2cnc(S(C)(=O)=O)nc2)C(C)C)cc1]

In some embodiments, the Degrader-Linker compound is represented by the structure of

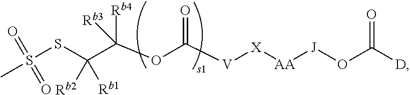

wherein R^{b1} and R^{b2} are each independently hydrogen, C₁-C₆ alkyl, or both R^{b1} and R^{b2}, together with the carbon to which they are attached, comprise a C₄-C₆ cycloalkyl or a C₄-C₆ heterocycloalkyl; R^{b3} and R^{b4} are each independently hydrogen or C₁-C₆ alkyl; and subscript s1 is 0. In some embodiments, U, X, AA, and J are each absent. In some embodiments, U, X, and AA are each absent. In some embodiments, the Degrader-Linker compound is represented by the structure of

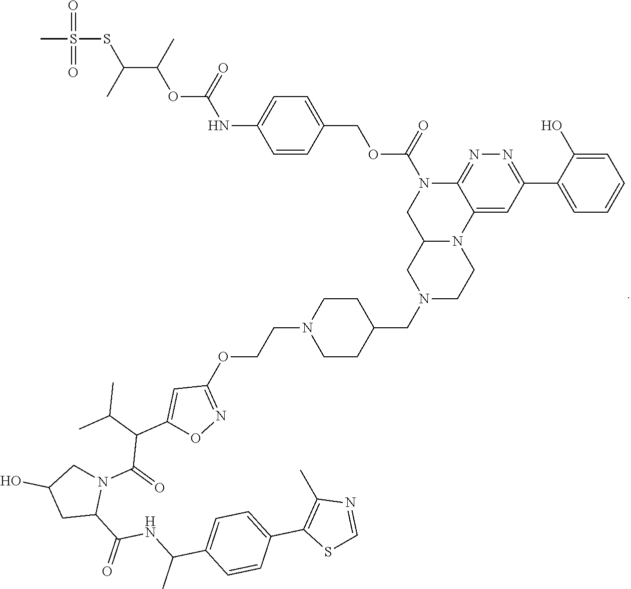 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6ccc(NC(=O)OC(C)C(C)SS(C)(=O)=O)cc6)CC5C4)CC3)no2)C(C)C)cc1]

In some embodiments, the Degrader-Linker compound is represented by the structure of

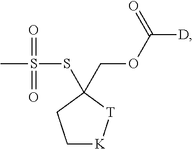

wherein T is absent or is —CH₂CH₂—and K is —CH₂— or —NH₂—. In some embodiments, T is absent. In some embodiments, T is —CH₂CH₂—. In some embodiments, K is —CH₂—. In some embodiments, K is —NH₂—.

In some embodiments, the Degrader-Linker compound is represented by the structure of

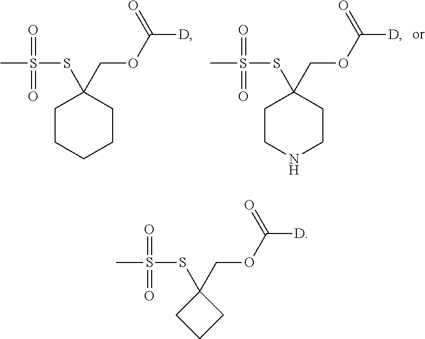

In some embodiments, the Degrader-Linker compound is represented by the structure of

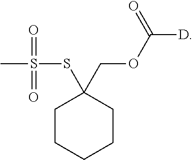

In some embodiments, the Degrader-Linker compound is represented by the structure of

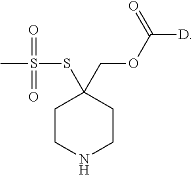

In some embodiments, the Degrader-Linker compound is represented by the structure of

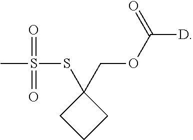

In some embodiments, the Degrader-Linker compound is represented by the structure of

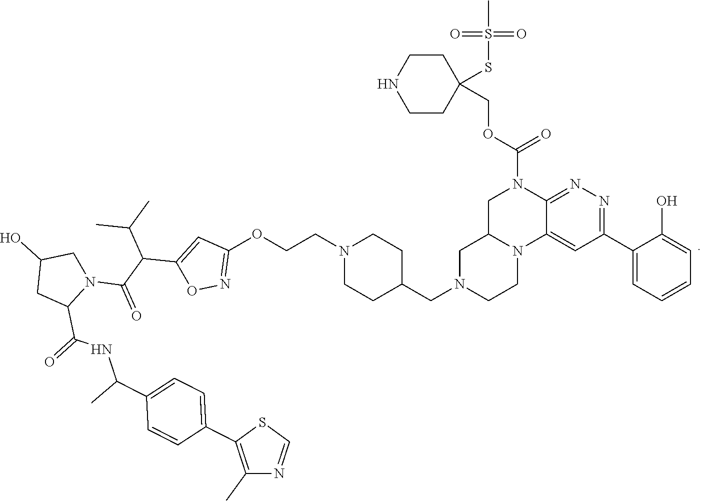 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCC6(SS(C)(=O)=O)CCNCC6)CC5C4)CC3)no2)C(C)C)cc1 ; "."]

In some embodiments, the Degrader-Linker compound is represented by the structure of

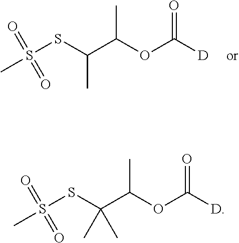

In some embodiments, the Degrader-Linker compound is represented by the structure of

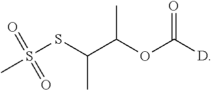

In some embodiments, the Degrader-Linker compound is represented by the structure of

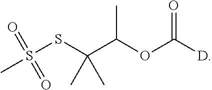

In some embodiments, the Degrader-Linker compound is represented by the structure of

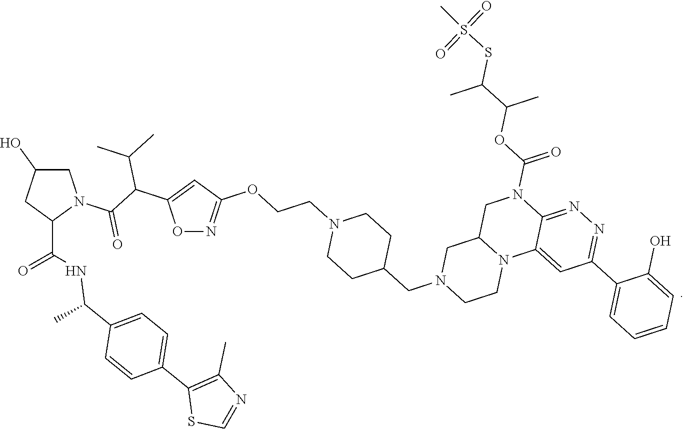 [SMILES: Cc1ncsc1-c1ccc([C@H](C)NC(=O)C2CC(O)CN2C(=O)C(c2cc(OCCN3CCC(CN4CCN5c6cc(-c7ccccc7O)nnc6N(C(=O)OC(C)C(C)SS(C)(=O)=O)CC5C4)CC3)no2)C(C)C)cc1 ; "."]

In some embodiments, the Degrader-Linker compound is represented by the structure of

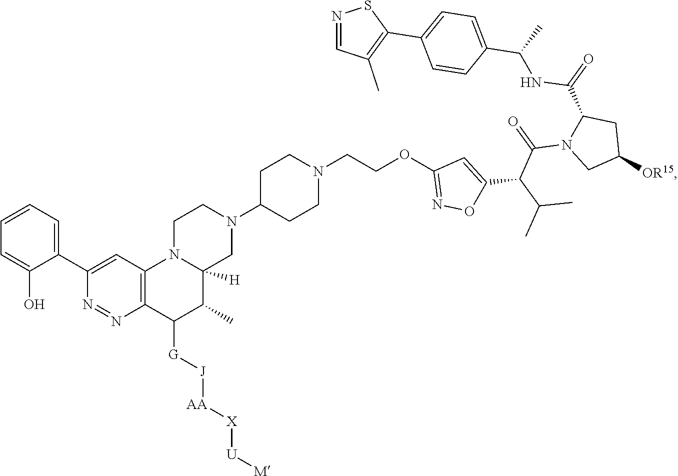

wherein R¹⁵ is hydrogen or —PO₃H₂. In some embodiments, the Degrader-Linker compound is represented by the structure of

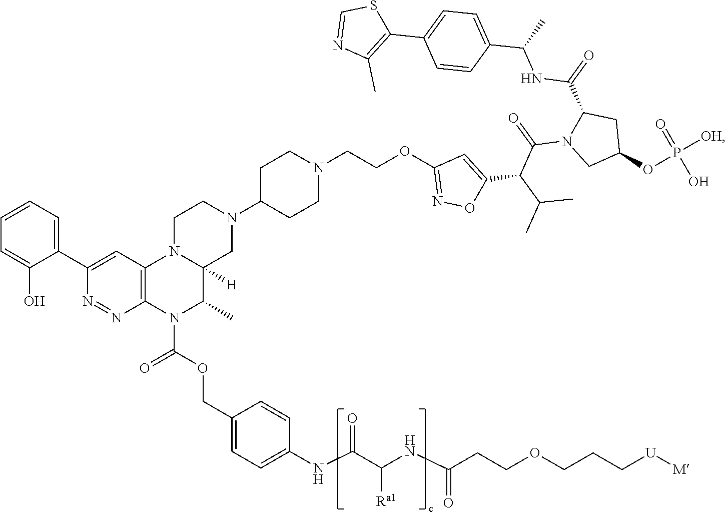

wherein subscript c is an integer ranging between 1 and 4; subscript w is an integer ranging between 1 and 8; and U is absent or is —CH₂—(C═O)—(NH)—. In some embodiments, each R^{a1} is an independently selected side chain of alanine, valine, or citrulline. In some embodiments, subscript c is 2. In some embodiments, subscript w is 1. In other embodiments, subscript w is 8. In some embodiments, U is absent. In other embodiments, U is —CH₂—(C═O)—(NH)—. In some embodiments, M′ is

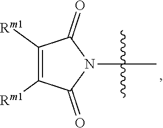

wherein each R^{m1} and R^{m2}is independently hydrogen, halogen, or —S-Ph. In some embodiments, each R^{m1} is hydrogen.

In some embodiments, the Degrader-Linker compound is represented by the structure of.

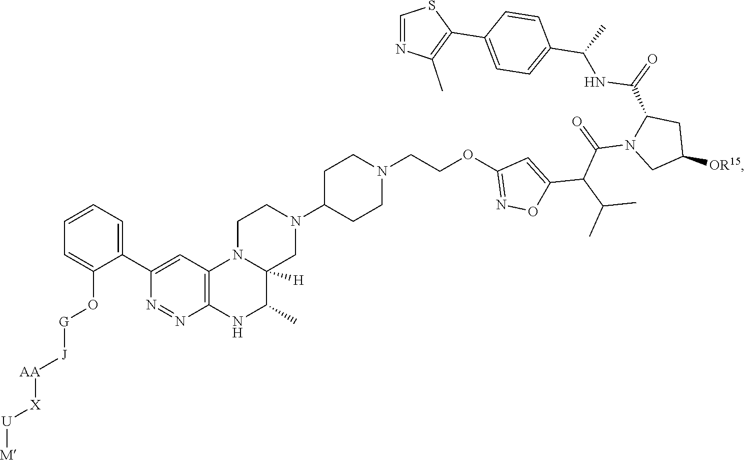

wherein R¹⁵ is hydrogen or —PO₃H₂. In some embodiments, the Degrader-Linker compound is represented by the structure of:

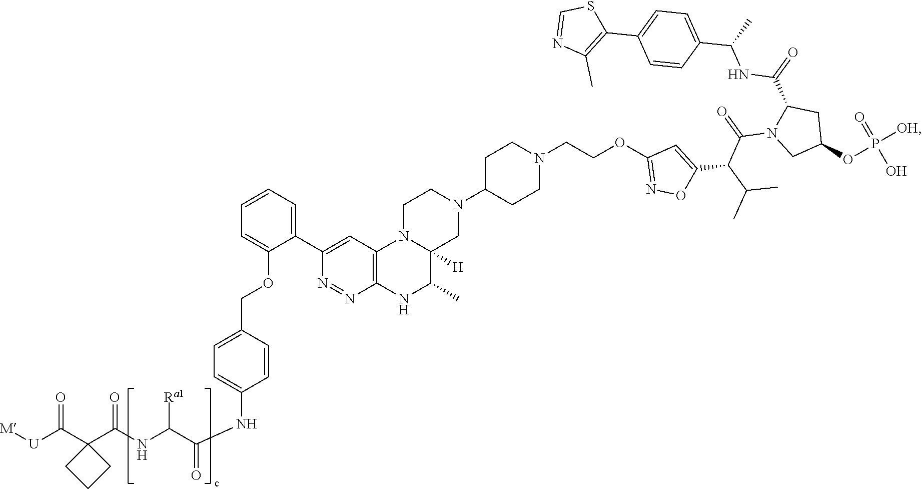

wherein subscript c is an integer ranging between 1 and 4; and U is —(CH₂CH₂O)_{w}CH₂CH₂(NH)—, wherein subscript w is an integer ranging from 1 to 4. In some embodiments, subscript c is 1 and R^{a1} is the side chain of citrulline. In some embodiments, U is —(CH₂CH₂O)_{w}CH₂CH₂(NH)— and subscript w is 4. In some embodiments, subscript c is 1 and R^{a1} is a side chain of citrulline. In some embodiments, M′ is

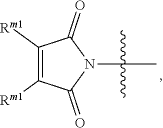

wherein each R^{m1} and R^{m2}is independently hydrogen, halogen, or —S-Ph. In some embodiments, each R^{a1} is hydrogen.

In some embodiments, the Degrader-Linker compound is represented by the structure of:

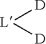

wherein,
each D is independently a degrader compound of Formula (I):

PTM-ULM (I),
wherein,
   PTM is a moiety of Formula IA:

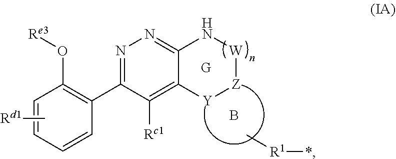

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
   ULM is a small molecule E3 Ubiquitin Ligase binding moiety that binds a Von Hippel-Lindau E3 Ubiquitin Ligase or a Cereblon E3 Ubiquitin Ligase;
L′ is a linker precursor of Formula (iii)

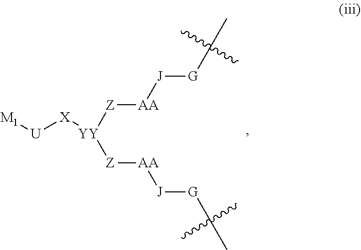

wherein
   M′ is selected from the group consisting of:

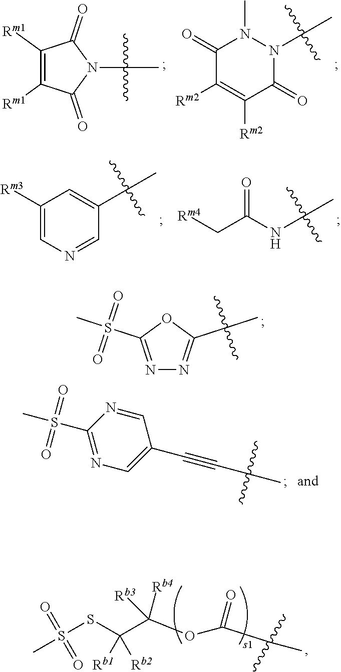

wherein
   each R^{m1} and R^{m2} is independently hydrogen, halogen, or —S-Ph;
   R^{m3} and R^{m4} are each halogen;
   R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
   R^{b3} and R^{b4} are each independently hydrogen or C₁-C₆ alkyl,
   subscript s1 is 0 or 1;
   wherein the wavy line of M′ indicates the point of covalent attachment to the remainder of the structure of L′ and the wavy line of Formula (iii) indicates the point of covalent attachment to the degrader compound (D); and
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1}is hydrogen, —COOH, —C(O)NHCH₃, or —(C(O)NHCH₂)ₓ(CH₂OCH₂)_{y}R², wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—OC(O)—, wherein R^{x5} is hydrogen or C₁-C₆ alkyl and R^{x6} is hydrogen, C₁-C₆ alkyl, or

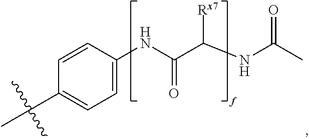

wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of:

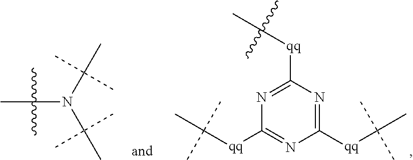

wherein
   each qq is independently —N(R^{y1})— or —O—, wherein R^{y1} is hydrogen or C₁-C₆ alkyl;
   the wavy line indicates the point of attachment to X, when present, or U, when X is absent, or M′, when X and U are absent; and
   each dashed line indicates the point of attachment to ZZ when ZZ is present, or AA when ZZ is absent, or J when AA and ZZ are absent, or G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of:

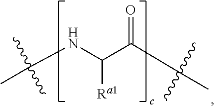

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

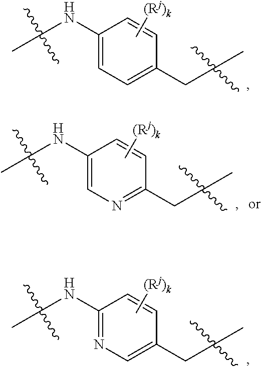

wherein
   R^{j3} is hydrogen, C₁-C₆ alkyl, or C₃-C₆ cycloalkyl;
   each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
   subscript m1 is an integer ranging from 1 to 6;
   each R^{j} is independently halogen, C₁-C₆ alkyl, or —C(O)NH(C₁-C₆ alkyl);
   subscript k is an integer ranging from 0 to 4;
each G is independently absent,

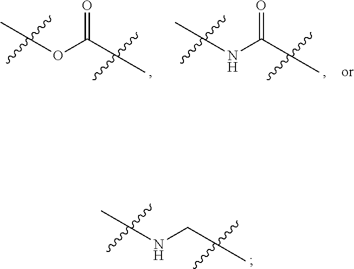

and
wherein the wavy line to each G of Formula (iii) indicates the point of covalent attachment to D.

In some embodiments, L′ has the structure of:

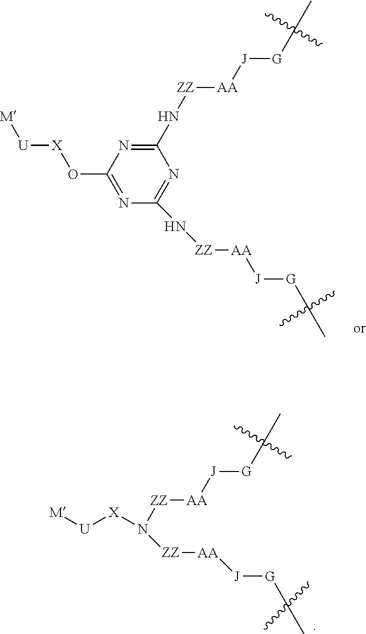

In some embodiments, L′ has the structure of:

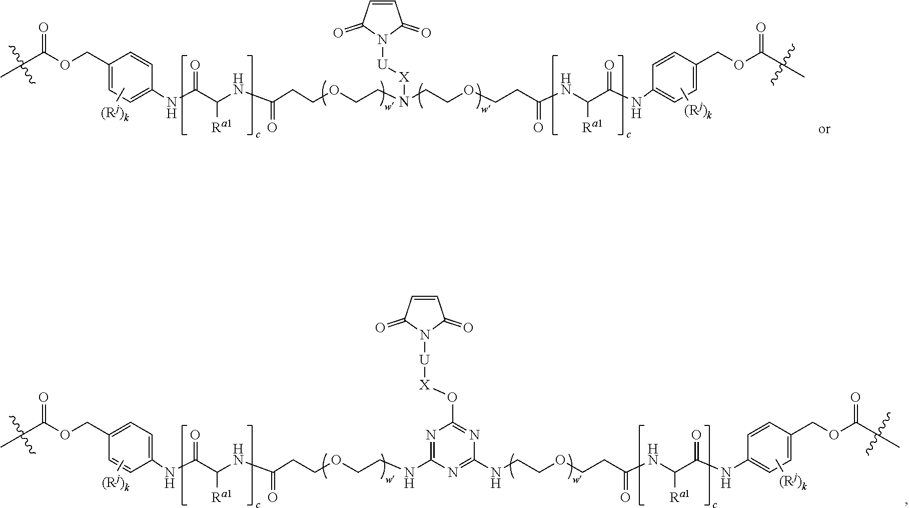

wherein
U is absent or is —(CH₂)_{b}(R^{v1})ₛₛ(C═O)ᵤ(NH)ᵥ—, wherein subscript b is 0, 1, 2, 3, 4, or 5; subscript ss is 0 or 1; subscript u is 0 or 1; subscript v is 0 or 1; R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or —(CH₂CH₂O)_{w}CH₂CH₂(NH)—, wherein subscript w is an integer ranging from 1 to 16;
X is absent or is —(CH₂CH₂O)_{w}CH₂CH₂—(C═O)_{d}—, wherein subscript w is an integer ranging from 0 to 8 and subscript d is 0 or 1;
subscript c is an integer ranging from 1 to 12 and each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine;
each R³ is independently fluorine, chlorine, methyl, or —C(O)NHCH₃; and subscript k is an integer ranging from 0 to 4.

In some embodiments, the Degrader-Linker compound is represented by the structure of:

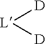

wherein,
each D is independently a degrader compound of Formula (I):

PTM-ULM (I),
wherein,
   PTM is a moiety of Formula IA:

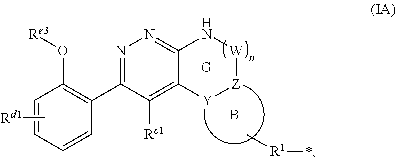

wherein,
   R¹ is a covalent bond, or chemical moiety that links PTM and ULM;
   * is a point of attachment to ULM;
   n=0-3;
   each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
   R^{c1} and R^{d1} are independently H, deuterium, Halo, C₁₋₃ alkyl, C₁₋₃ haloalkyl, or C₁₋₄ alkoxyl;
   R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃. 8 substituted cyclcoalkyl, C₃₋₈ heterocyclcoalkyl, or C₃₋₈ substituted heterocyclcoalkyl;
   Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
   B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase;
L′ is a linker precursor of Formula (iib):

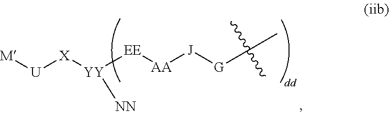

wherein
M′ is selected from the group consisting of:

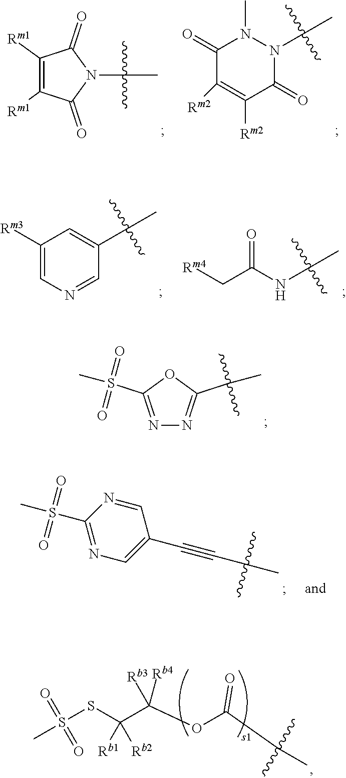

wherein
each R^{m1} and R^{m2} is independently hydrogen, halogen, or —S-Ph;
R^{m3} and R^{m4} are each halogen;
R^{b1} and R^{b2} are each independently hydrogen or C₁-C₆ alkyl, or R^{b1} and R^{b2}together with the carbon to which they are attached form a C₄-C₆ cycloalkyl or a C₄-C₆ heterocycloalkyl;
R^{b3} and R^{b4} are each independently hydrogen or C₁-C₆ alkyl, subscript s1 is 0 or 1;
wherein the wavy line of M′ indicates the point of covalent attachment to the remainder of the structure of L′ and the wavy line of Formula (iii) indicates the point of covalent attachment to the degrader compound (D); and
U is absent or is —(CH₂)_{b}(R^{v1})ₛₛ(C═O)ᵤ(NH)ᵥ— wherein
   subscript b is 0, 1, 2, 3, 4, or 5;
   subscript ss is 0 or 1;
   subscript u is 0 or 1;
   subscript v is 0 or 1;
   R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
   —(CH₂CH₂O)_{w}CH₂CH₂(NH)—, wherein subscript w is an integer ranging from 1 to 16;
X is absent or is
   —(CH₂CH₂O)_{w}CH₂CH₂—(C═O)_{d}—, wherein subscript w is an integer ranging from 0 to 16 and subscript d is 0 or 1;
   —CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
   —C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
   -heterocycloalkyl-O(CH₂)ᵢC(O)—, wherein subscript i is an integer ranging from 0 to 6;
   —C(O)—C(R^{x5})(R^{x6})—, wherein R^{x5} is hydrogen or C₁-C₆ alkyl and R^{x6} is hydrogen, C₁-C₆ alkyl, or

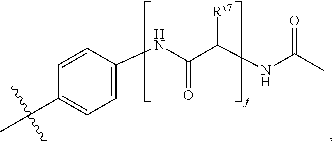

wherein subscript f is an integer ranging from 0 to 4 and each R^{x7} is independently hydrogen, —COOH, —NH₂, C₁-C₆ alkyl, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit having the structure of:

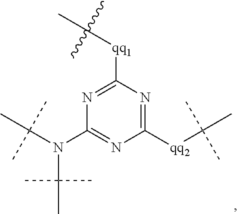

wherein
   qq₁ and qq₂ are each independently —N(R^{y1})— or —O—, wherein R^{y1} is hydrogen or C₁-C₆ alkyl, and
the wavy line of YY indicates the point of attachment to X, when present, or to U when X is absent, or to M when X and U are absent; and
the dashed lines of YY indicate the point of attachment to EE when EE is present, or to AA when EE is absent, or to J when AA and EE are absent, or to G when AA, EE, and J are absent, and to NN;
NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
   each EE is independently absent or —(CH₂CH₂O)_{x′}CH₂CH₂C(O)—, wherein
   subscript x′ is an integer ranging from 0 to 16;
each AA is independently absent or has the structure of:

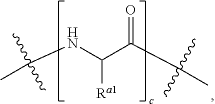

wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of:

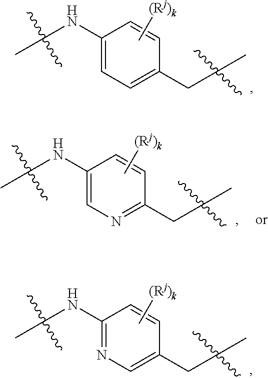

wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
each G is independently absent,

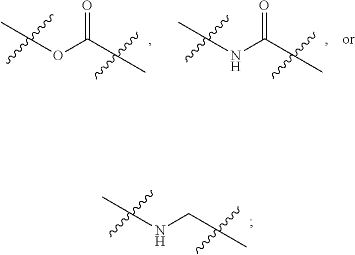

and
   wherein the wavy line to each G of Formula (iib) indicates the point of covalent attachment to an independently selected D.

In some embodiments, L′ is a linker precursor of Formula (iii) and is covalently attached to two degrader compounds (D) of Formula (I). In some embodiments, L′ is a linker precursor of Formula (iii) and is covalently attached to two of the same degrader compounds of Formula (I).

In some embodiments, L′ is a linker precursor of Formula (iii) and is covalently attached to two degrader compounds of Formula (I), and each degrader compound is a different degrader compound of Formula (I). For example, in some embodiments, L′ is a linker precursor of Formula (iii) and is covalently attached to a degrader compound of Formula (I) wherein the ULM of Formula (I) binds Von Hippel-Lindau E3 Ubiquitin Ligase and to a degrader compound of Formula (I) wherein the ULM of Formula (I) binds Cereblon E3 Ubiquitin Ligase. In some such embodiments, the ULM of Formula (I) that binds Von Hippel-Lindau E3 Ubiquitin Ligase has the Formula ULM-I-VHL, as described herein, and the ULM of (I) that binds Cereblon E3 Ubiquitin Ligase has the formula of ULM-II-CRBN, as described herein.

In some embodiments, L′ is a linker precursor of Formula (iii) and is covalently attached to two different degrader compounds of Formula (I) that both bind Von Hippel-Lindau E3 Ubiquitin Ligase. In some embodiments, L is covalently attached to two different degrader compounds of Formula (I) that both bind Cereblon E3 Ubiquitin Ligase.

In some embodiments, L′ is a linker precursor of Formula (iib) and is covalently attached to two degrader compounds (D) of Formula (I). In some embodiments, L′ is a linker precursor of Formula (iib) and is covalently attached to two of the same degrader compounds of Formula (I).

In some embodiments, L′ is a linker precursor of Formula (iib) and is covalently attached to two degrader compounds of Formula (I), and each degrader compound is a different degrader compound of Formula (I). For example, in some embodiments, L′ is a linker precursor of Formula (iib) and is covalently attached to a degrader compound of Formula (I) wherein the ULM of Formula (I) binds Von Hippel-Lindau E3 Ubiquitin Ligase and to a degrader compound of Formula (I) wherein the ULM of Formula (I) binds Cereblon E3 Ubiquitin Ligase. In some such embodiments, the ULM of Formula (I) that binds Von Hippel-Lindau E3 Ubiquitin Ligase has the Formula ULM-I-VHL, as described herein, and the ULM of (I) that binds Cereblon E3 Ubiquitin Ligase has the formula of ULM-II-CRBN, as described herein.

In some embodiments, L′ is a linker precursor of Formula (iib) and is covalently attached to two different degrader compounds of Formula (I) that both bind Von Hippel-Lindau E3 Ubiquitin Ligase. In some embodiments, L is covalently attached to two different degrader compounds of Formula (I) that both bind Cereblon E3 Ubiquitin Ligase.

In some embodiments, provided is a drug-linker compound (i.e., a degrader-linker compound) of Table 2, or a pharmaceutically acceptable salt thereof.

TABLE 2
Degrader-Linker Compounds

| Com- | |
|---|---|
| pound | |
| No. | Compound Structure |
| 2.01 | 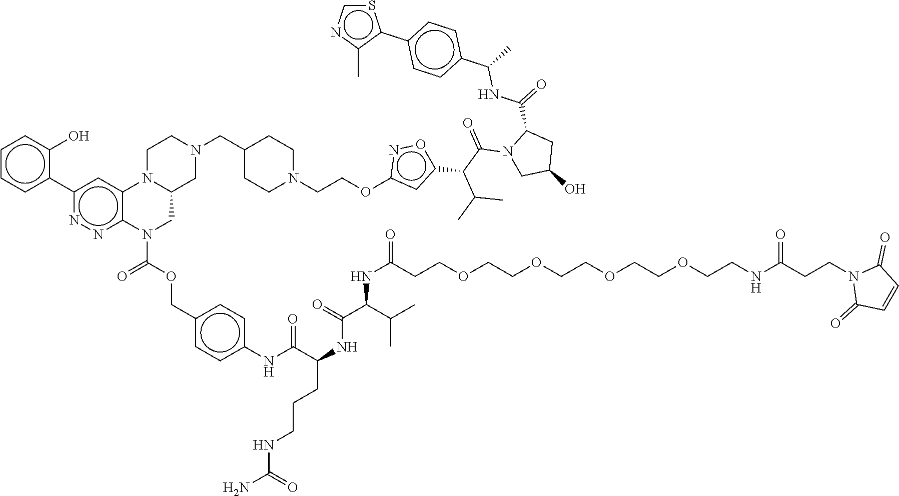 |
| 2.02 | 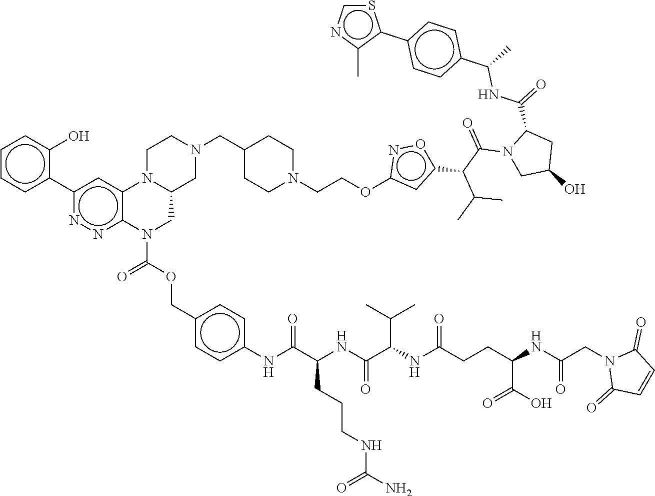 |
| 2.03 | 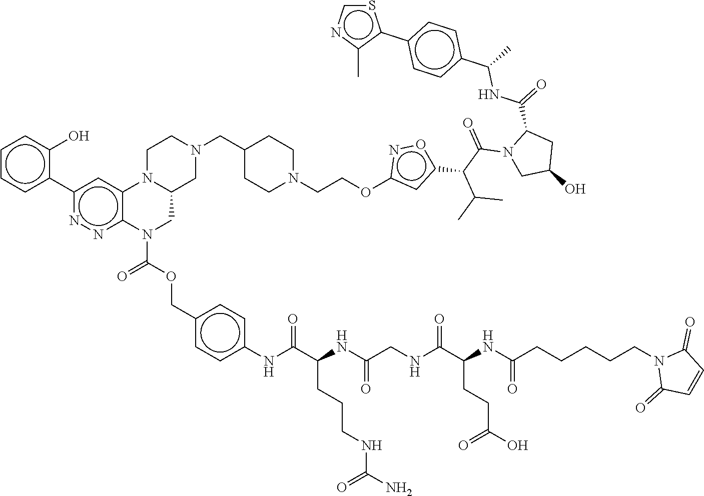 |
| 2.04 | 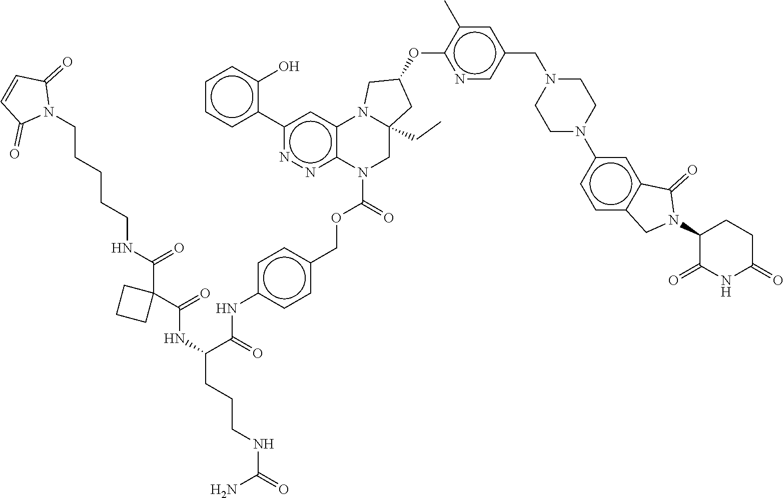 |
| 2.05 | 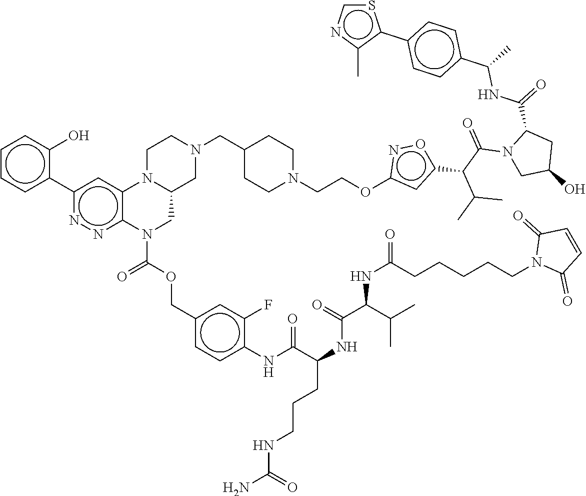 |
| 2.06 | 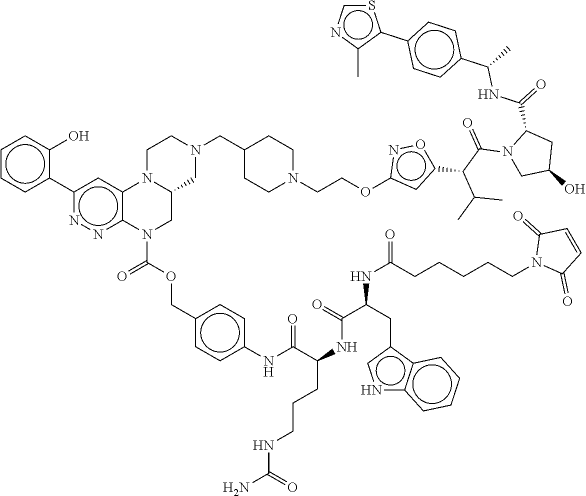 |
| 2.07 | 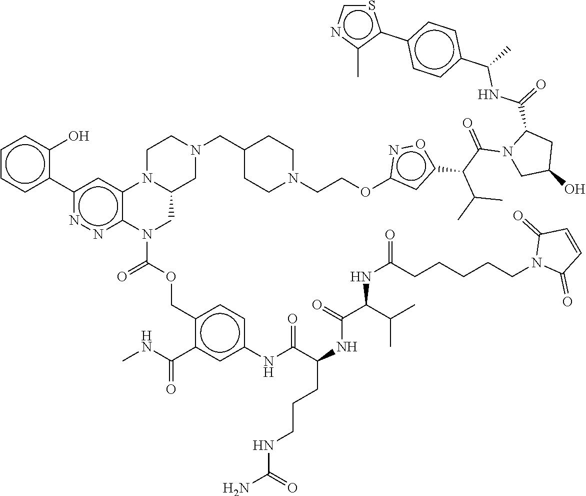 |
| 2.08 | 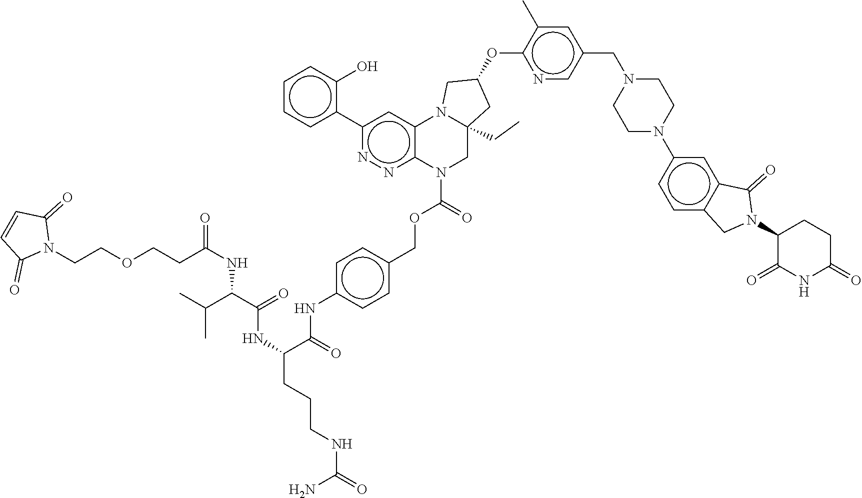 |
| 2.09 | 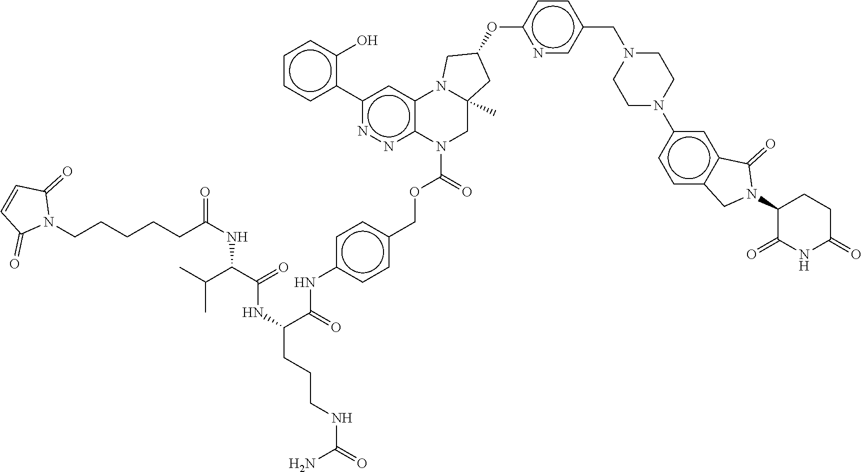 |
| 2.10 | 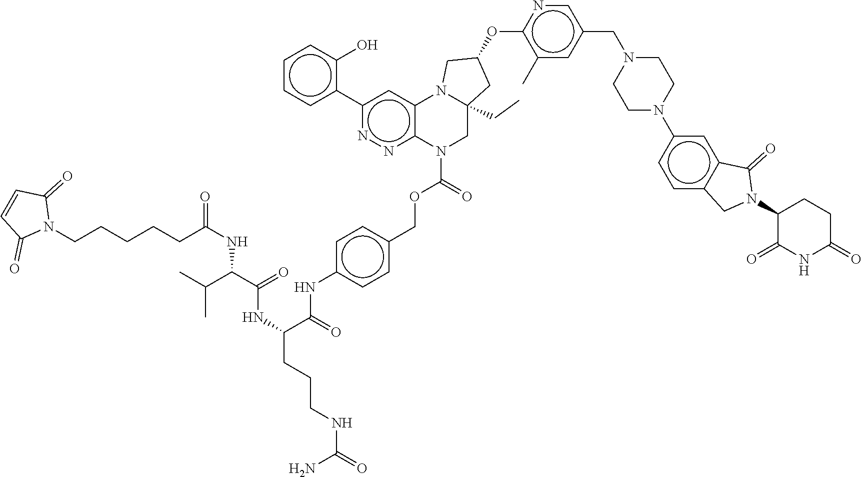 |
| 2.11 | 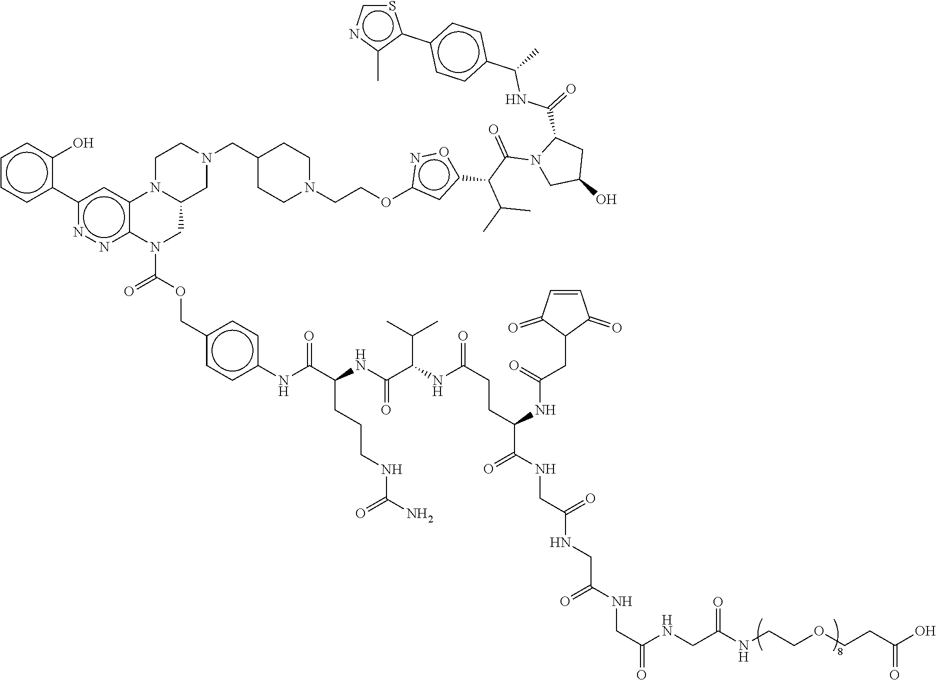 |
| 2.12 | 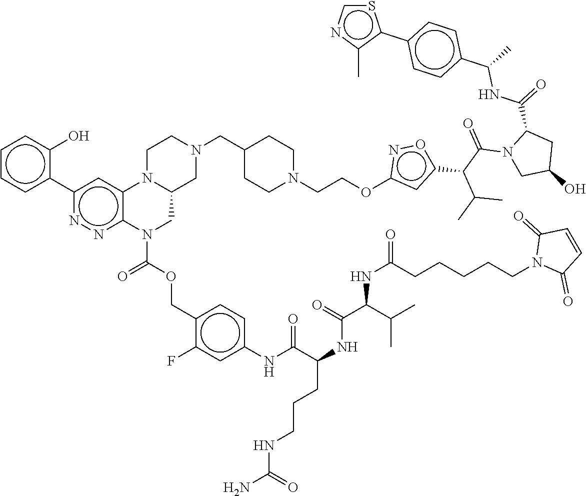 |
| 2.13 | 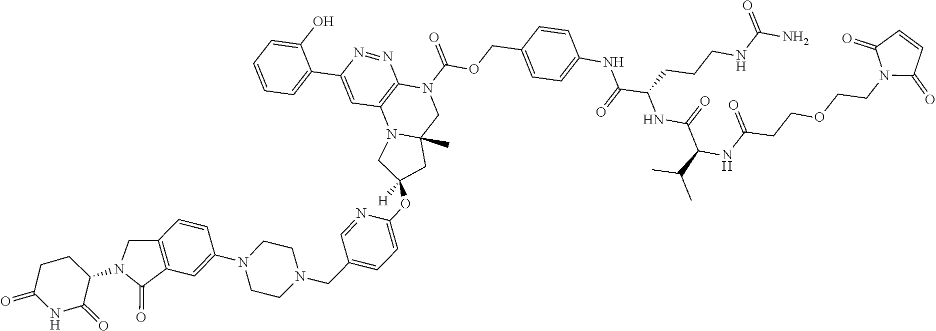 [SMILES: CC(C)[C@H](NC(=O)CCOCCN1C(=O)C=CC1=O)C(=O)N[C@@H](CCCNC(N)=O)C(=O)Nc1ccc(COC(=O)N2C[C@]3(C)C[C@@H](Oc4ccc(CN5CCN(c6ccc7c(c6)C(=O)N([C@H]6CCC(=O)NC6=O)C7)CC5)cn4)CN3c3cc(-c4ccccc4O)nnc32)cc1] |
| 2.14 | 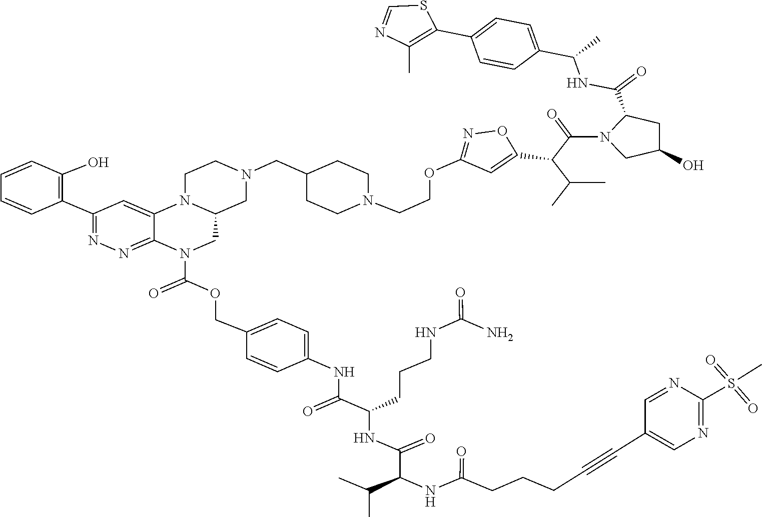 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCC#Cc7cnc(S(C)(=O)=O)nc7)C(C)C)cc6)C[C@H]5C4)CC3)no2)C(C)C)cc1] |
| 2.15 | 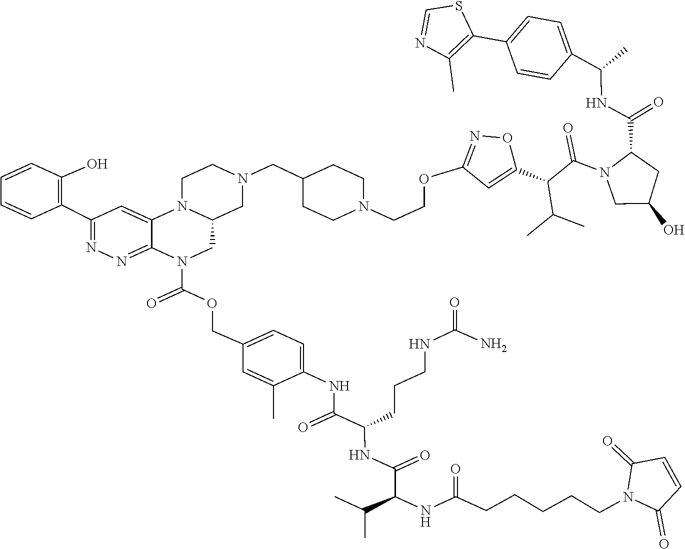 [SMILES: Cc1cc(COC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)ccc1NC(=O)[C@H](CCCNC(N)=O)NC(=O)[C@@H](NC(=O)CCCCCN1C(=O)C=CC1=O)C(C)C] |
| 2.16 | 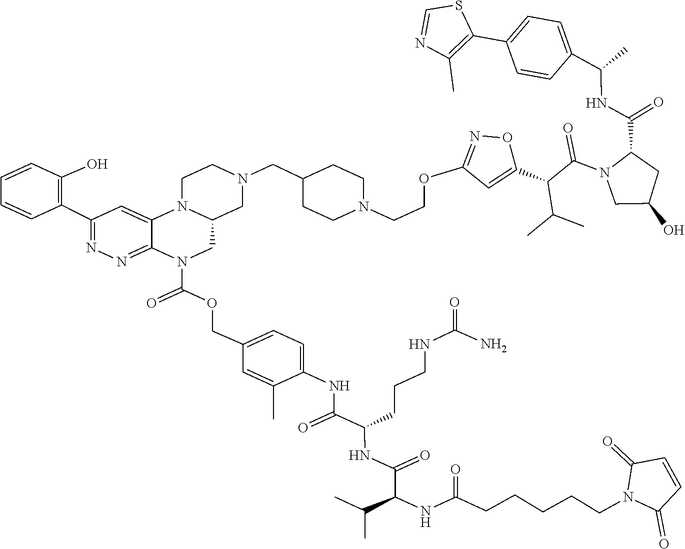 [SMILES: Cc1cc(COC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)ccc1NC(=O)[C@H](CCCNC(N)=O)NC(=O)[C@@H](NC(=O)CCCCCN1C(=O)C=CC1=O)C(C)C] |
| 2.17 | 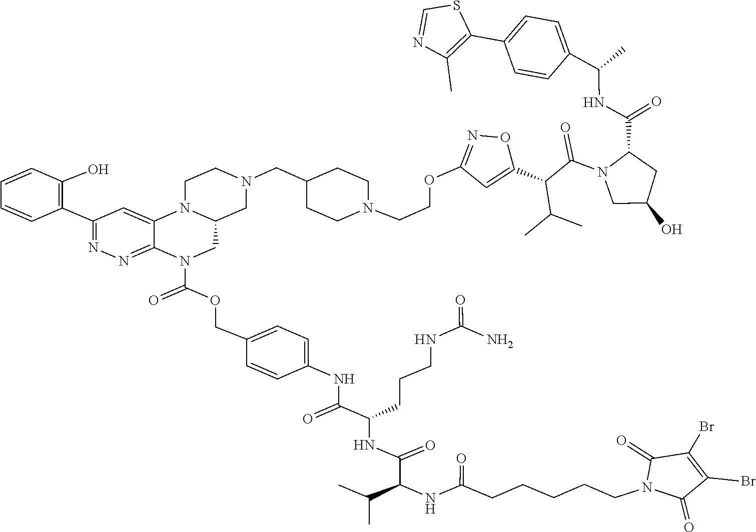 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C(Br)=C(Br)C7=O)C(C)C)cc6)C[C@H]5C4)CC3)no2)C(C)C)cc1] |
| 2.18 | 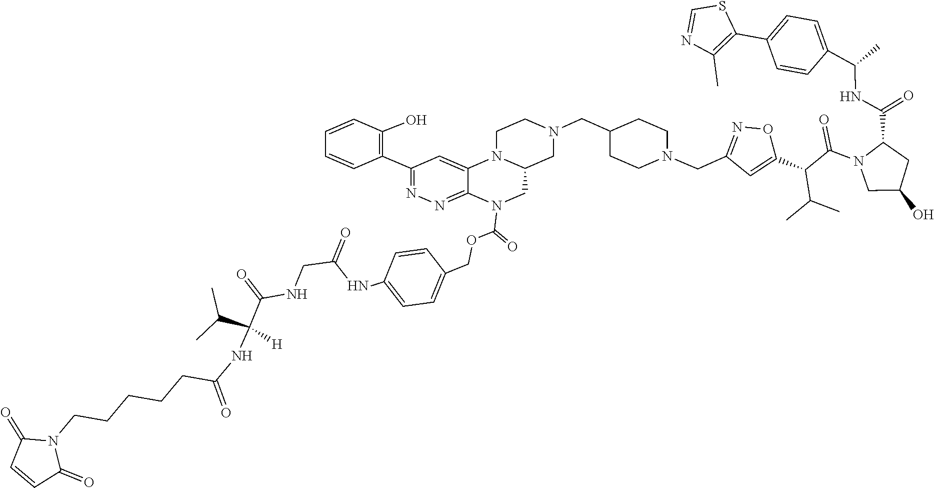 [SMILES: Cc1ncsc1-c1ccc([C@H](C)NC(=O)[C@@H]2C[C@@H](O)CN2C(=O)[C@@H](c2cc(CN3CCC(CN4CCN5c6cc(-c7ccccc7O)nnc6N(C(=O)OCc6ccc(NC(=O)CNC(=O)[C@H](NC(=O)CCCCCN7C(=O)C=CC7=O)C(C)C)cc6)C[C@H]5C4)CC3)no2)C(C)C)cc1] |
| 2.19 | 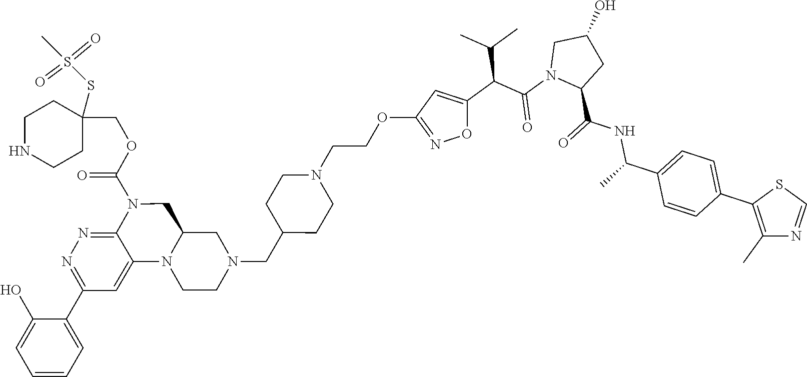 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCC6(SS(C)(=O)=O)CCNCC6)C[C@H]5C4)CC3)no2)C(C)C)cc1] |
| 2.20 | 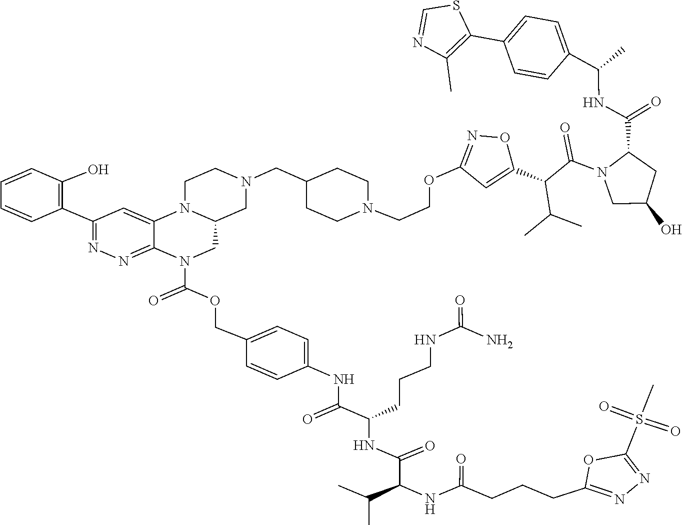 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCc7nnc(S(C)(=O)=O)o7)C(C)C)cc6)C[C@H]5C4)CC3)no2)C(C)C)cc1] |
| 2.21 | 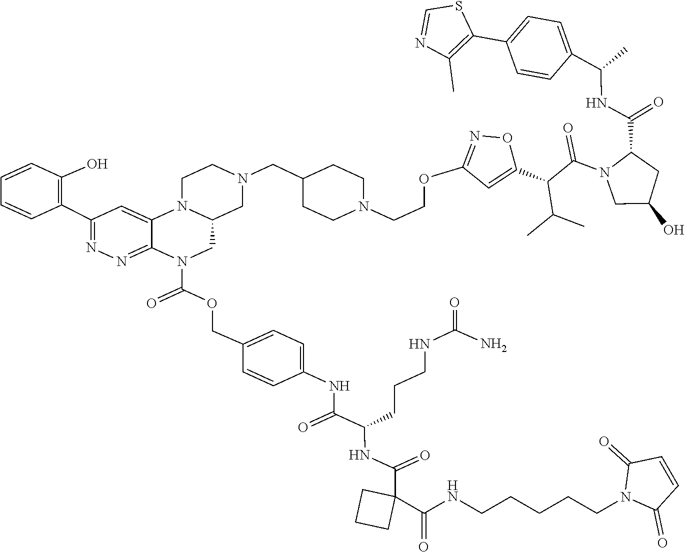 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)C7(C(=O)NCCCCCN8C(=O)C=CC8=O)CCC7)cc6)C[C@H]5C4)CC3)no2)C(C)C)cc1] |
| 2.22 | 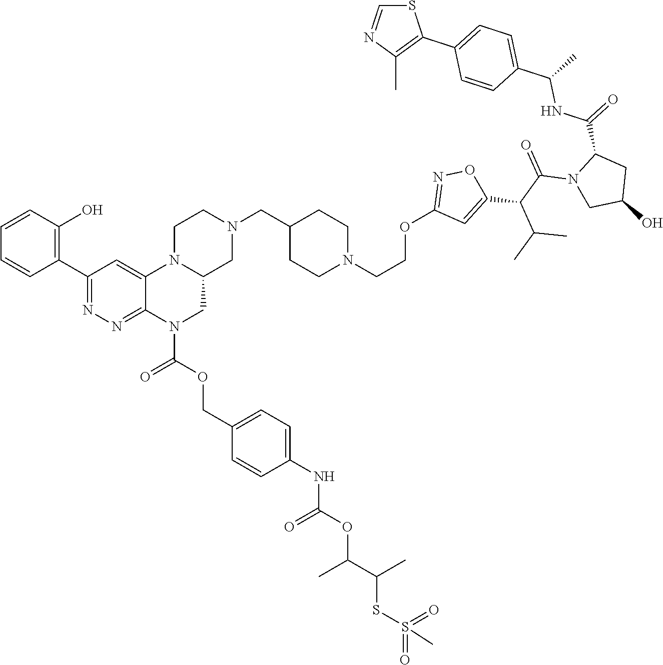 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)OC(C)C(C)SS(C)(=O)=O)cc6)C[C@H]5C4)CC3)no2)C(C)C)cc1] |
| 2.23 | 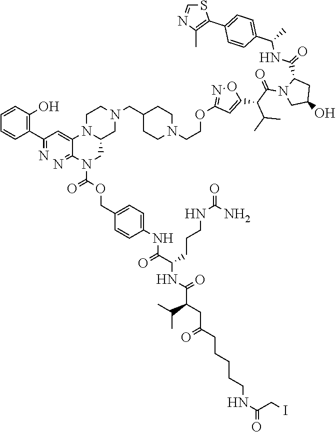 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](CC(=O)CCCCCNC(=O)CI)C(C)C)cc6)C[C@H]5C4)CC3)no2)C(C)C)cc1] |
| 2.24 | 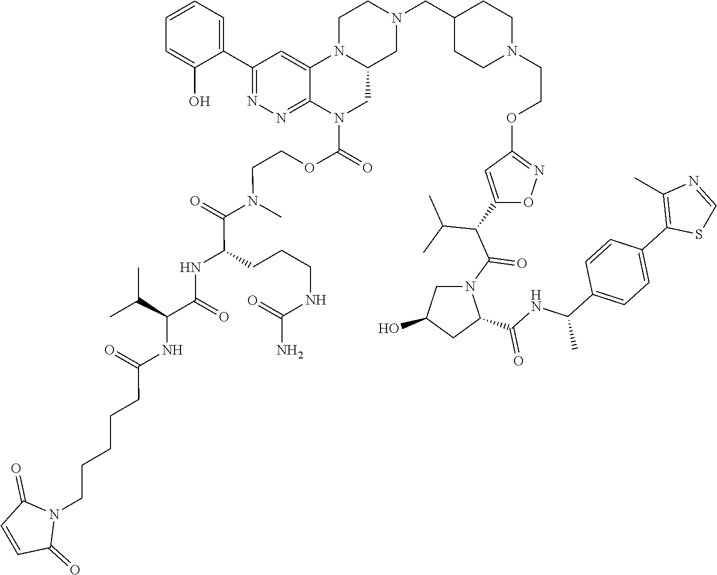 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCCN(C)C(=O)[C@H](CCCNC(N)=O)NC(=O)[C@@H](NC(=O)CCCCCN6C(=O)C=CC6=O)C(C)C)C[C@H]5C4)CC3)no2)C(C)C)cc1] |
| 2.25 | 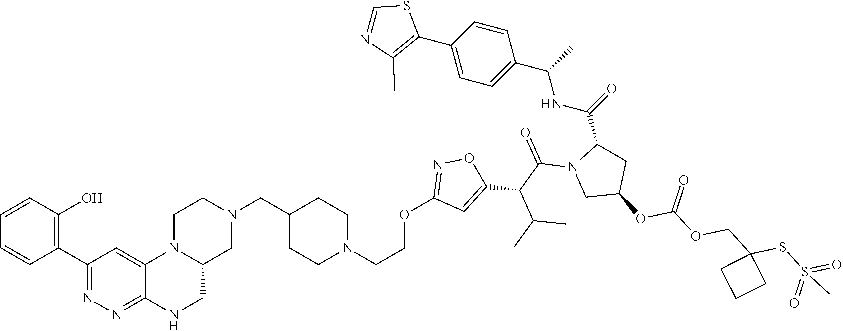 [SMILES: Cc1ncsc1-c1ccc([C@H](C)NC(=O)[C@@H]2C[C@@H](OC(=O)OCC3(SS(C)(=O)=O)CCC3)CN2C(=O)[C@@H](c2cc(OCCN3CCC(CN4CCN5c6cc(-c7ccccc7O)nnc6NC[C@H]5C4)CC3)no2)C(C)C)cc1] |
| 2.26 | 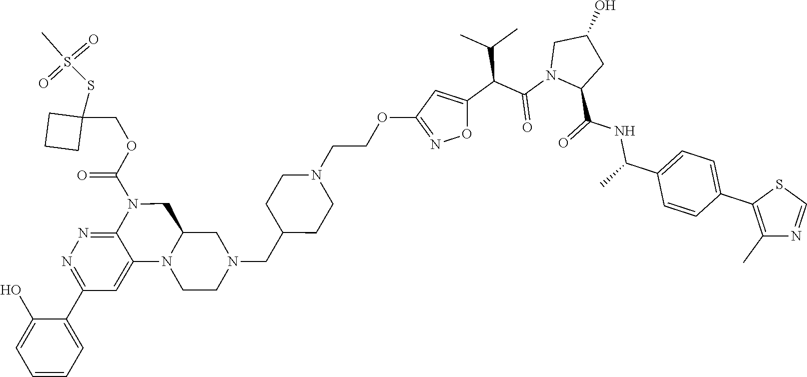 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCC6(SS(C)(=O)=O)CCC6)C[C@H]5C4)CC3)no2)C(C)C)cc1] |
| 2.27 | 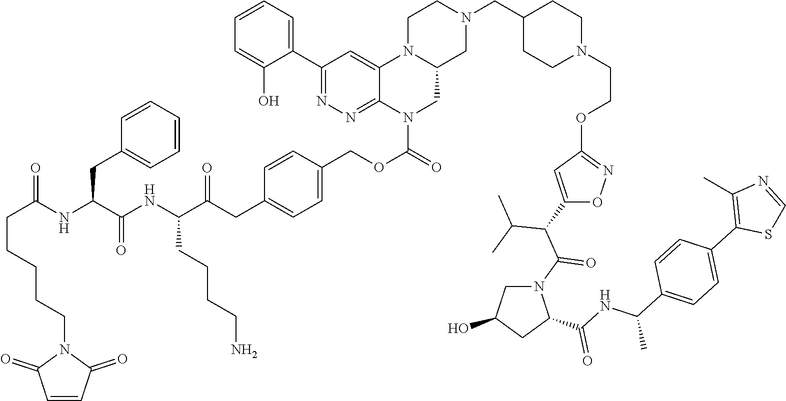 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(CC(=O)[C@H](CCCCN)NC(=O)[C@H](Cc7ccccc7)NC(=O)CCCCCN7C(=O)C=CC7=O)cc6)C[C@H]5C4)CC3)no2)C(C)C)cc1] |
| 2.28 | 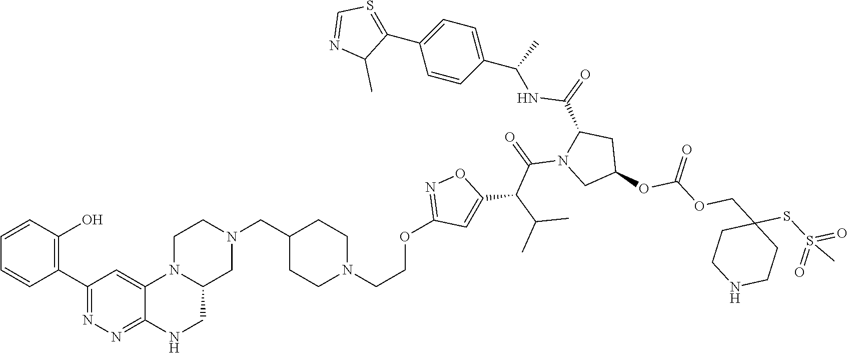 |
| 2.29 | 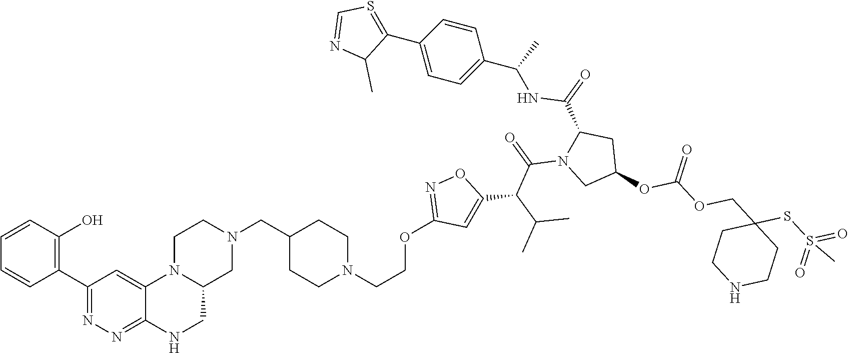 |
| 2.30 | 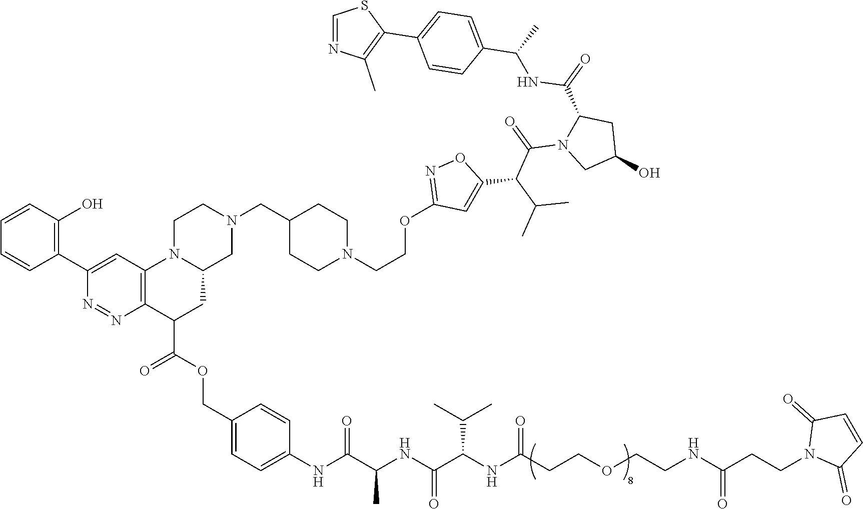 |
| 2.31 | 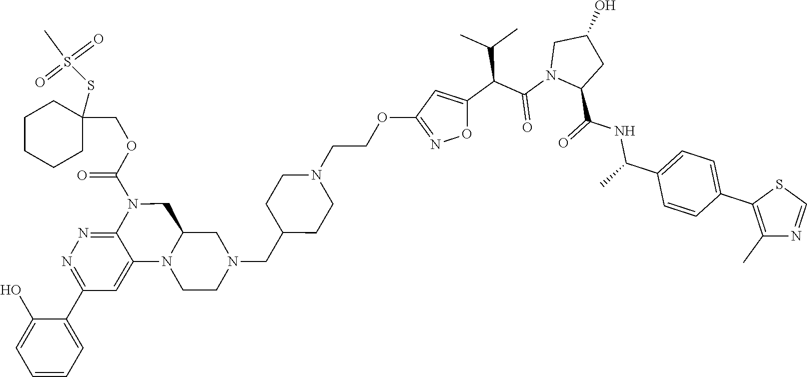 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCC6(SS(C)(=O)=O)CCCCC6)C[C@H]5C4)CC3)no2)C(C)C)cc1] |
| 2.32 | 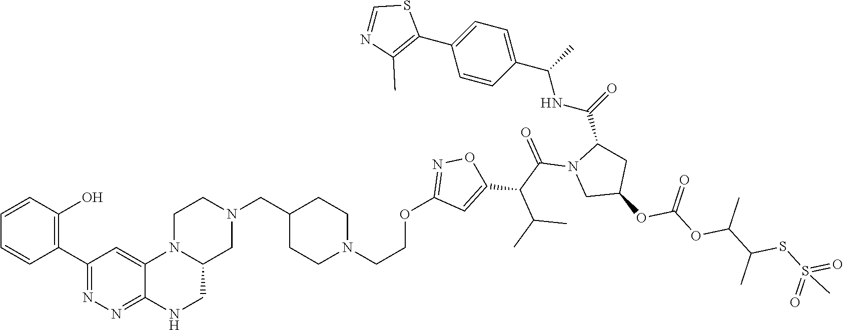 [SMILES: Cc1ncsc1-c1ccc([C@H](C)NC(=O)[C@@H]2C[C@@H](OC(=O)OC(C)C(C)SS(C)(=O)=O)CN2C(=O)[C@@H](c2cc(OCCN3CCC(CN4CCN5c6cc(-c7ccccc7O)nnc6NC[C@H]5C4)CC3)no2)C(C)C)cc1] |
| 2.33 | 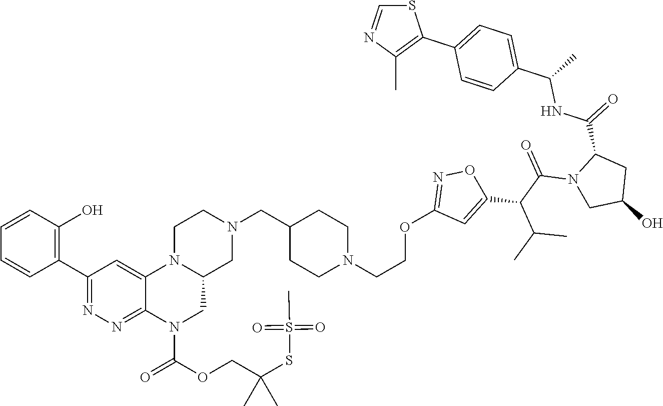 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCC(C)(C)SS(C)(=O)=O)C[C@H]5C4)CC3)no2)C(C)C)cc1] |
| 2.34 | 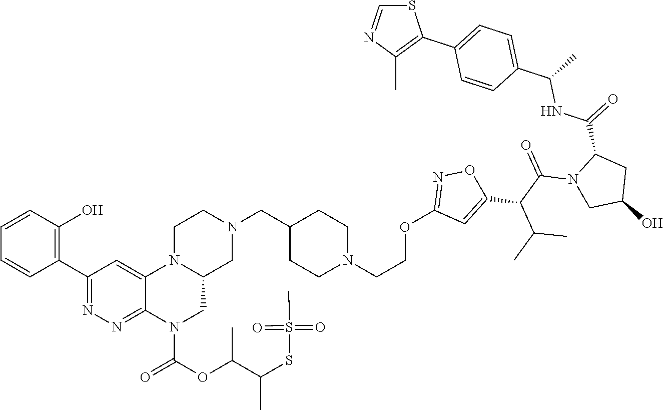 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OC(C)C(C)SS(C)(=O)=O)C[C@H]5C4)CC3)no2)C(C)C)cc1] |
| 2.35 | 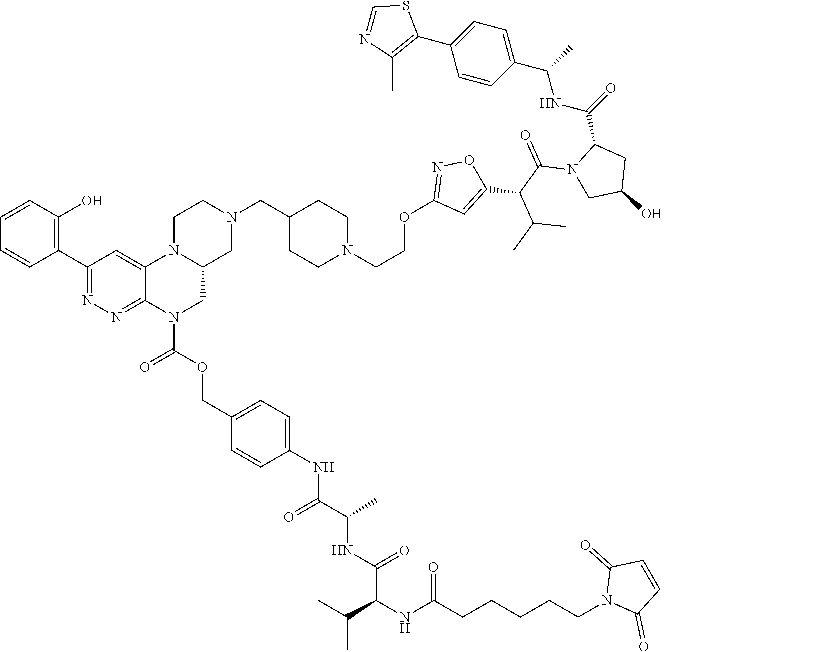 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](C)NC(=O)[C@@H](NC(=O)CCCCCN7C(=O)C=CC7=O)C(C)C)cc6)C[C@H]5C4)CC3)no2)C(C)C)cc1] |
| 2.36 | 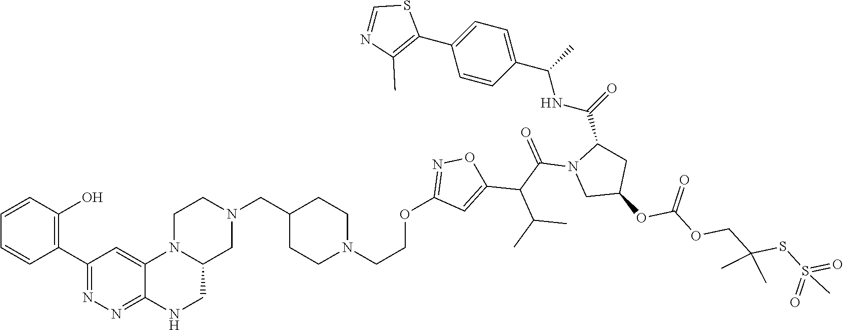 [SMILES: Cc1ncsc1-c1ccc([C@H](C)NC(=O)[C@@H]2C[C@@H](OC(=O)OCC(C)(C)SS(C)(=O)=O)CN2C(=O)C(c2cc(OCCN3CCC(CN4CCN5c6cc(-c7ccccc7O)nnc6NC[C@H]5C4)CC3)no2)C(C)C)cc1] |
| 2.37 | 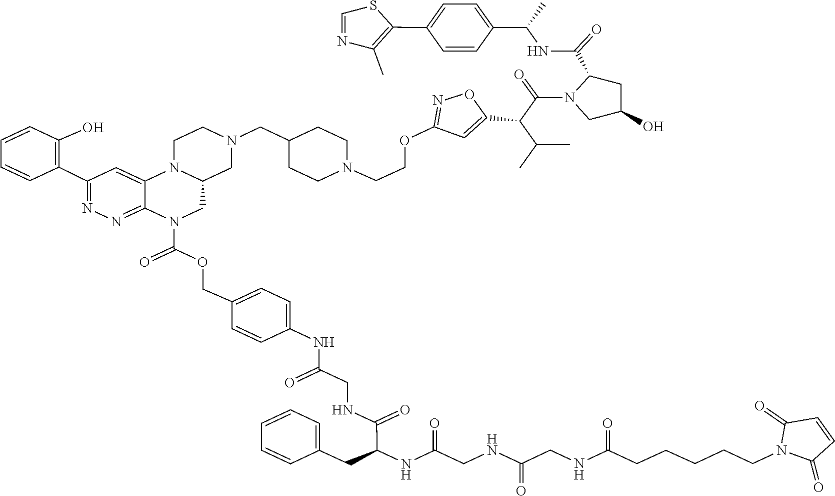 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)CNC(=O)[C@H](Cc7ccccc7)NC(=O)CNC(=O)CNC(=O)CCCCCN7C(=O)C=CC7=O)cc6)C[C@H]5C4)CC3)no2)C(C)C)cc1] |
| 2.38 | 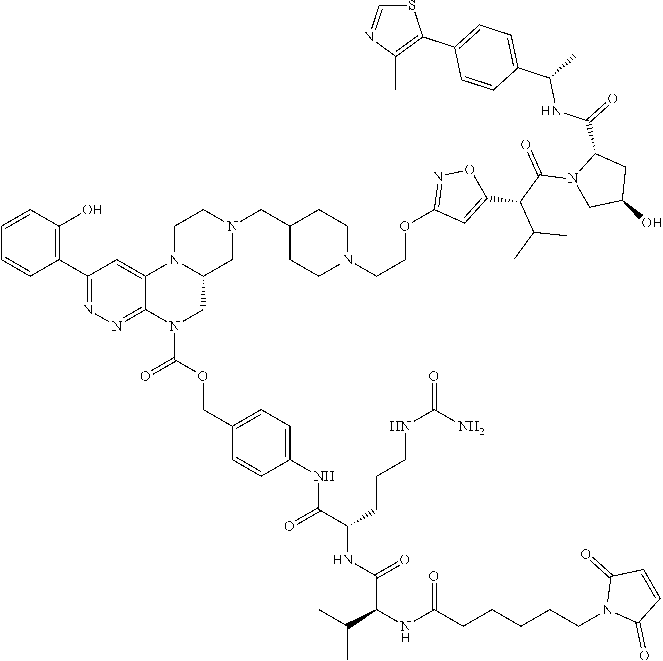 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C=CC7=O)C(C)C)cc6)C[C@H]5C4)CC3)no2)C(C)C)cc1] |
| 2.39 | 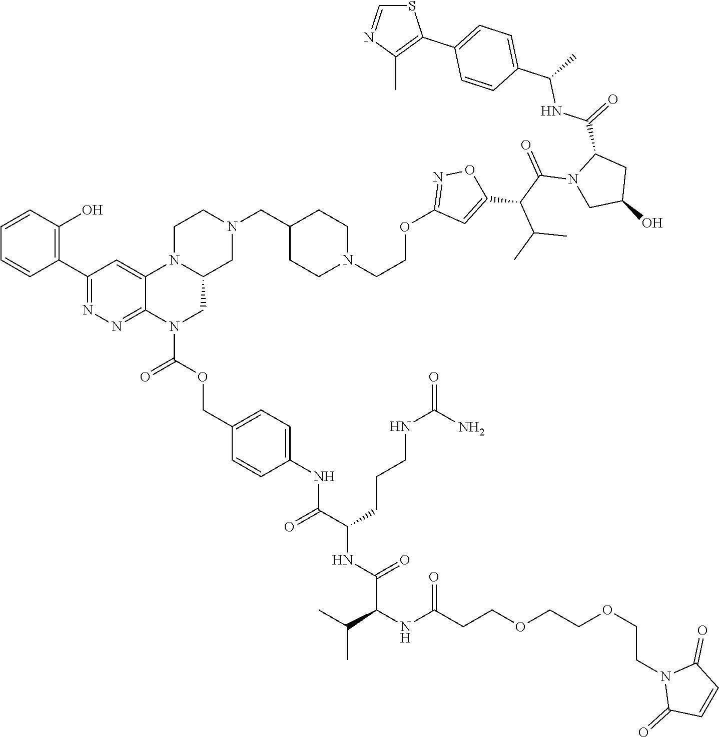 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCOCCOCCN7C(=O)C=CC7=O)C(C)C)cc6)C[C@H]5C4)CC3)no2)C(C)C)cc1] |
| 2.40 | 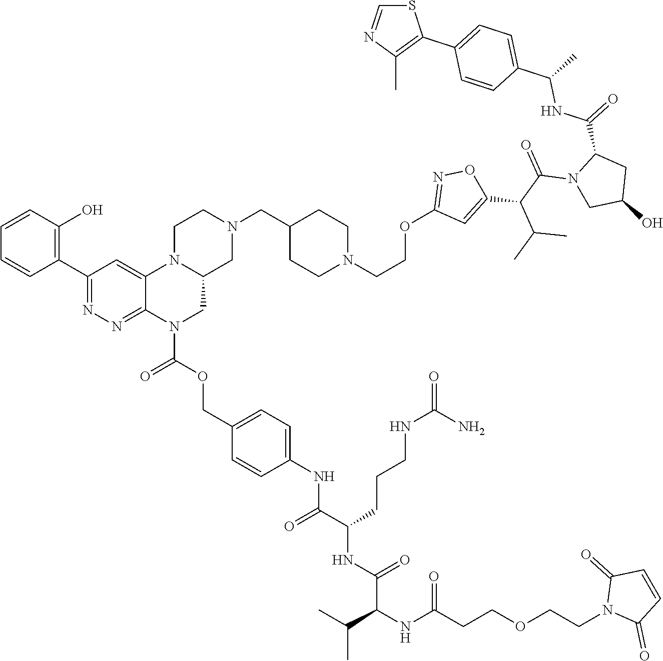 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCOCCN7C(=O)C=CC7=O)C(C)C)cc6)C[C@H]5C4)CC3)no2)C(C)C)cc1] |
| 2.41 | 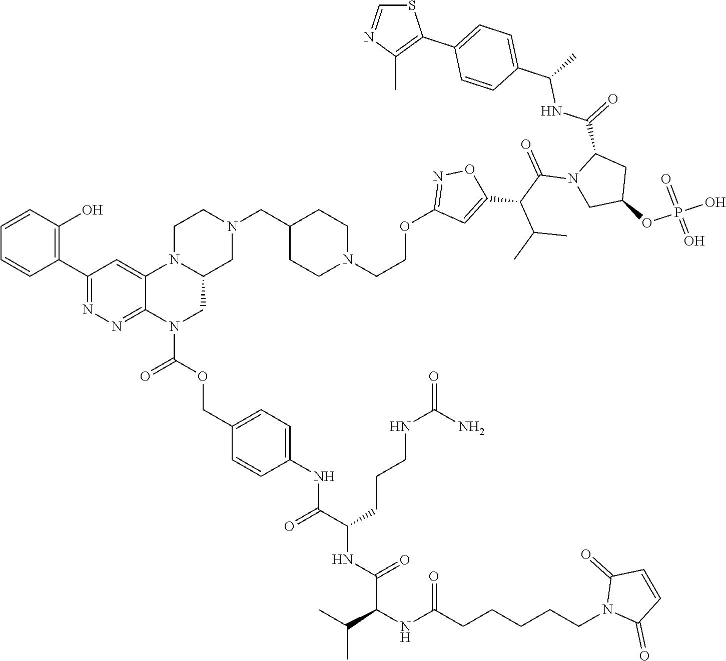 |
| 2.42 | 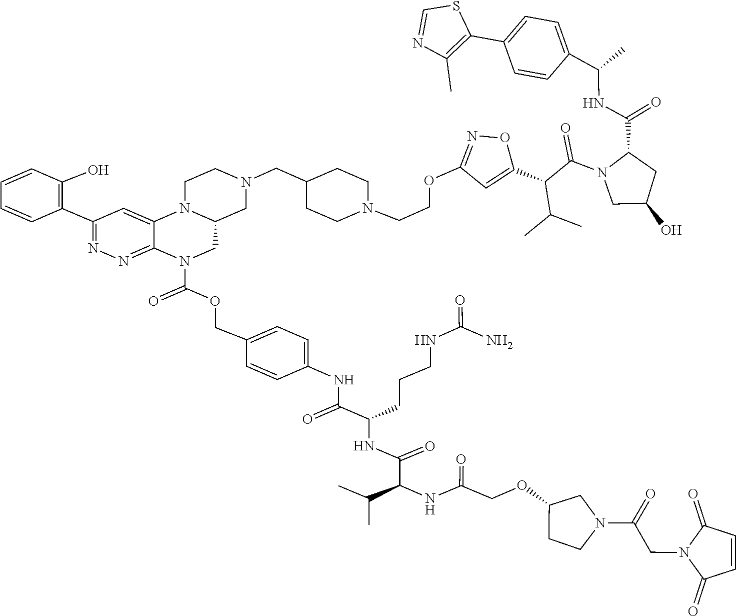 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CO[C@H]7CCN(C(=O)CN8C(=O)C=CC8=O)C7)C(C)C)cc6)C[C@H]5C4)CC3)no2)C(C)C)cc1] |
| 2.43 | 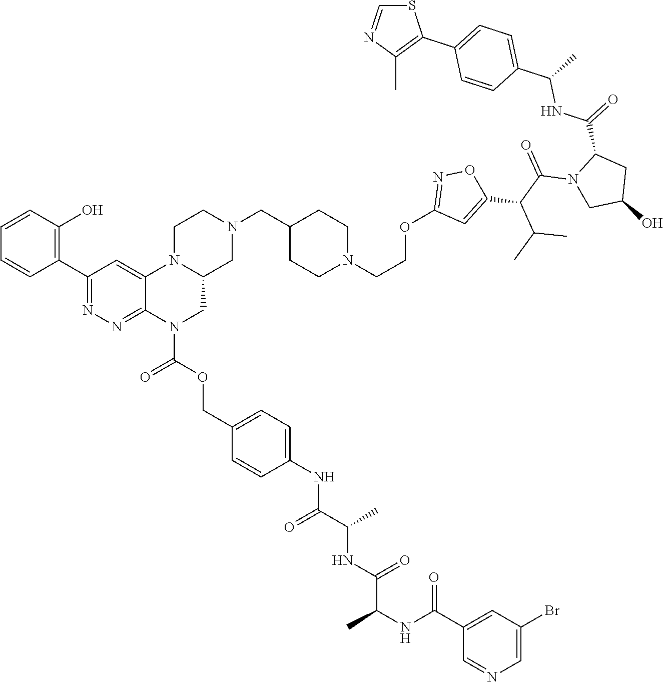 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](C)NC(=O)[C@H](C)NC(=O)c7cncc(Br)c7)cc6)C[C@H]5C4)CC3)no2)C(C)C)cc1] |
| 2.44 | 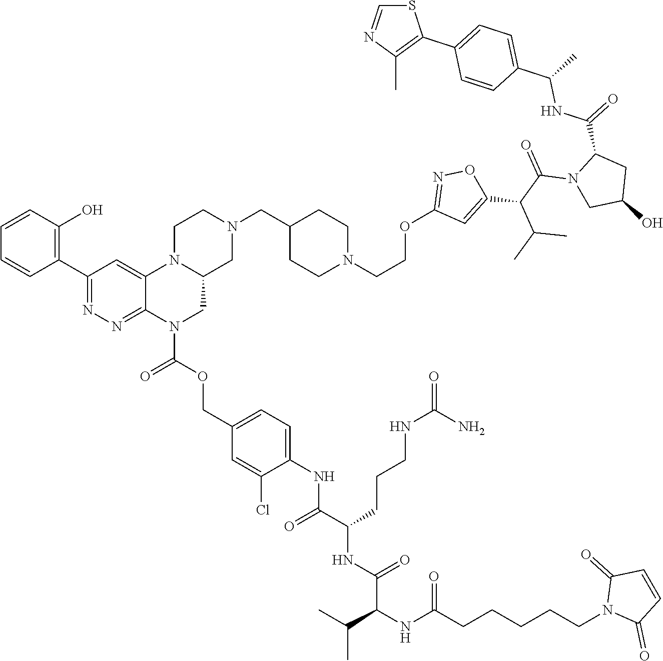 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C=CC7=O)C(C)C)c(Cl)c6)C[C@H]5C4)CC3)no2)C(C)C)cc1] |
| 2.45 | 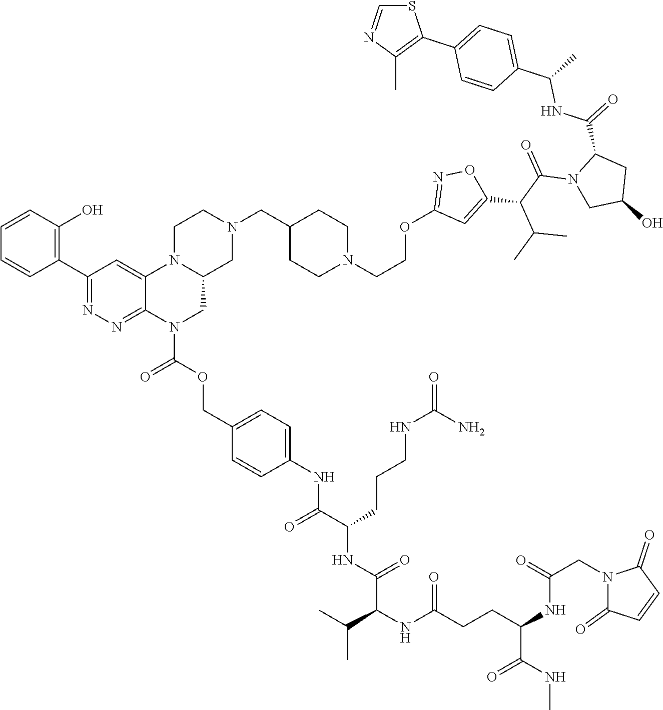 [SMILES: CNC(=O)[C@@H](CCC(=O)N[C@H](C(=O)N[C@@H](CCCNC(N)=O)C(=O)Nc1ccc(COC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)cc1)C(C)C)NC(=O)CN1C(=O)C=CC1=O] |
| 2.46 | 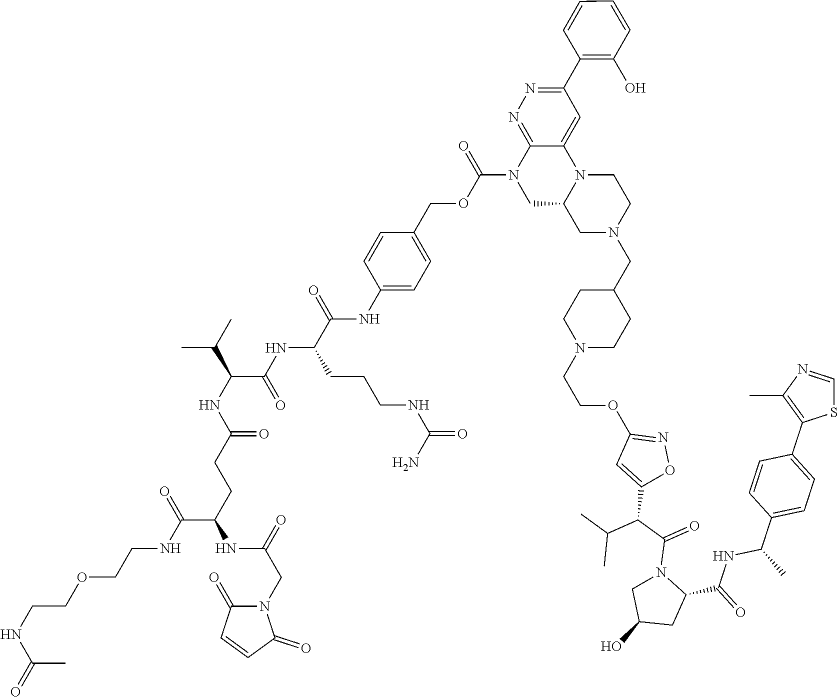 [SMILES: CC(=O)NCCOCCNC(=O)[C@@H](CCC(=O)N[C@H](C(=O)N[C@@H](CCCNC(N)=O)C(=O)Nc1ccc(COC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)cc1)C(C)C)NC(=O)CN1C(=O)C=CC1=O] |
| 2.47 | 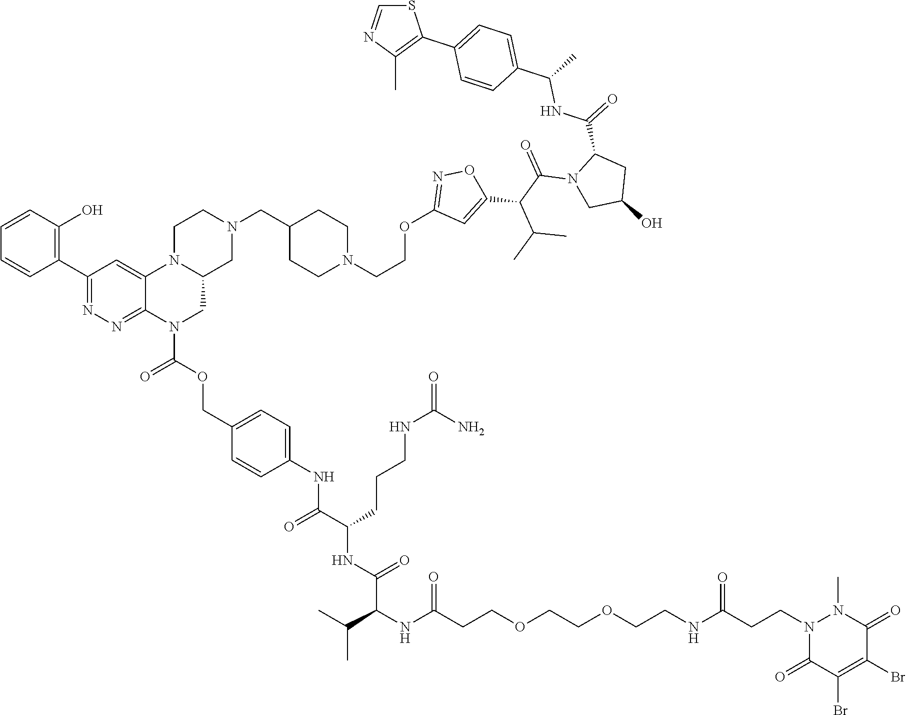 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCOCCOCCNC(=O)CCn7c(=O)c(Br)c(Br)c(=O)n7C)C(C)C)cc6)C[C@H]5C4)CC3)no2)C(C)C)cc1] |
| 2.48 | 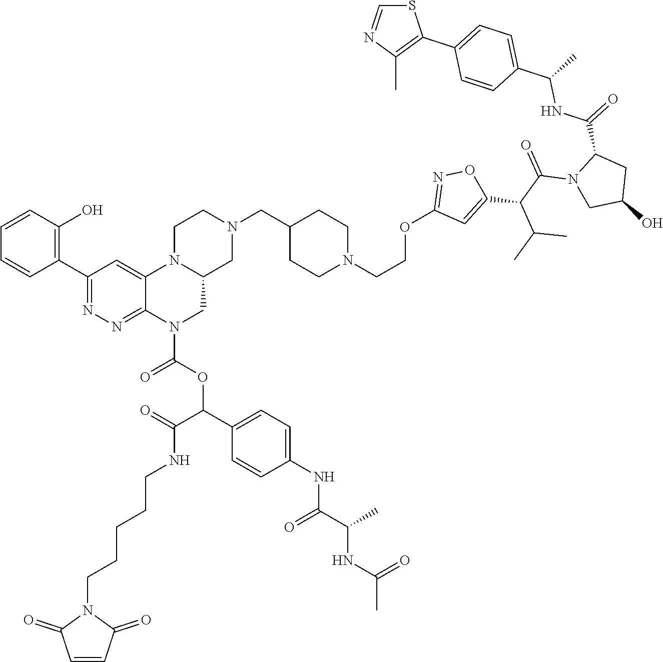 [SMILES: CC(=O)N[C@@H](C)C(=O)Nc1ccc(C(OC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)C(=O)NCCCCCN2C(=O)C=CC2=O)cc1] |
| 2.49 | 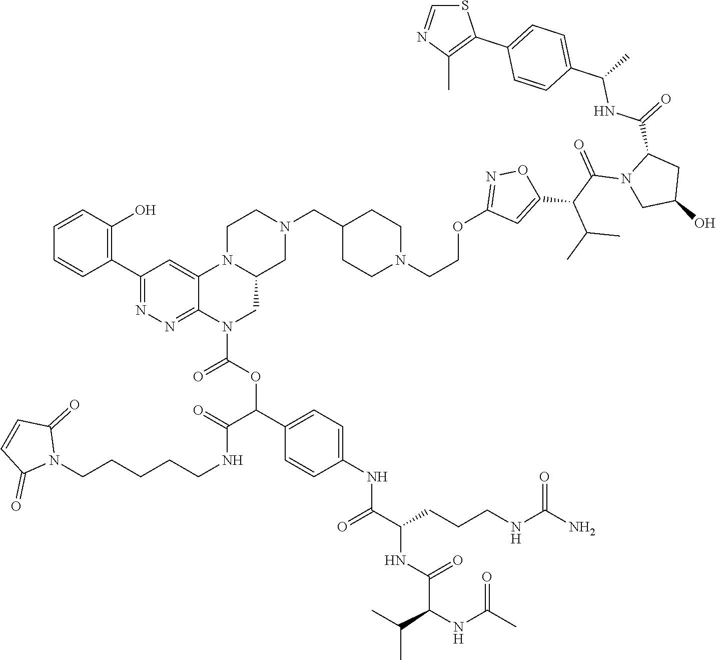 [SMILES: CC(=O)N[C@H](C(=O)N[C@@H](CCCNC(N)=O)C(=O)Nc1ccc(C(OC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)C(=O)NCCCCCN2C(=O)C=CC2=O)cc1)C(C)C] |
| 2.50 | 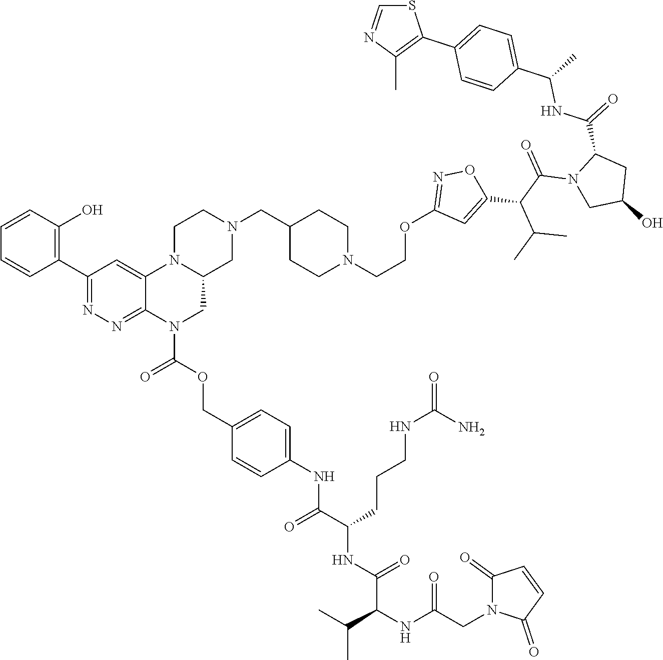 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CN7C(=O)C=CC7=O)C(C)C)cc6)C[C@H]5C4)CC3)no2)C(C)C)cc1] |
| 2.51 | 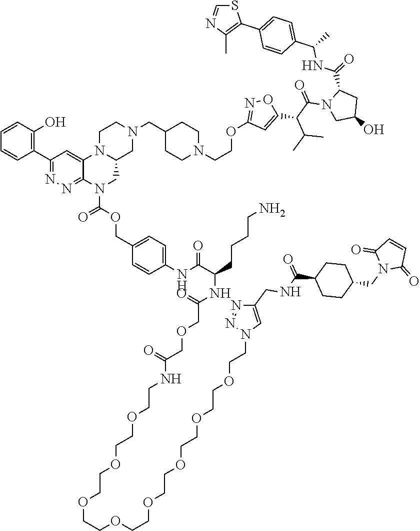 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@@H](CCCCN)NC(=O)COCC(=O)NCCOCCOCCOCCOCCOCCOCCOCCOCCn7cc(CNC(=O)[C@H]8CC[C@H](CN9C(=O)C=CC9=O)CC8)nn7)cc6)C[C@H]5C4)CC3)no2)C(C)C)cc1] |
| 2.52 | 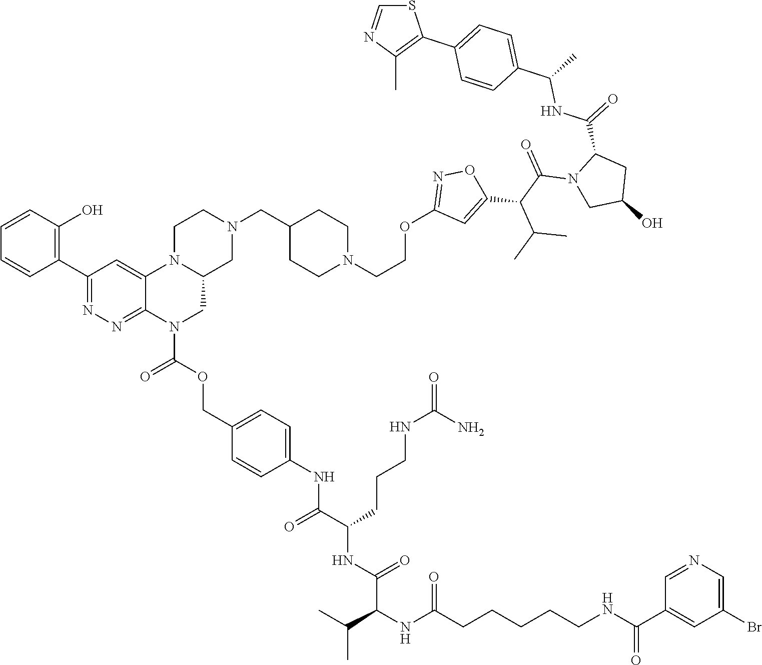 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCNC(=O)c7cncc(Br)c7)C(C)C)cc6)C[C@H]5C4)CC3)no2)C(C)C)cc1] |
| 2.53 | 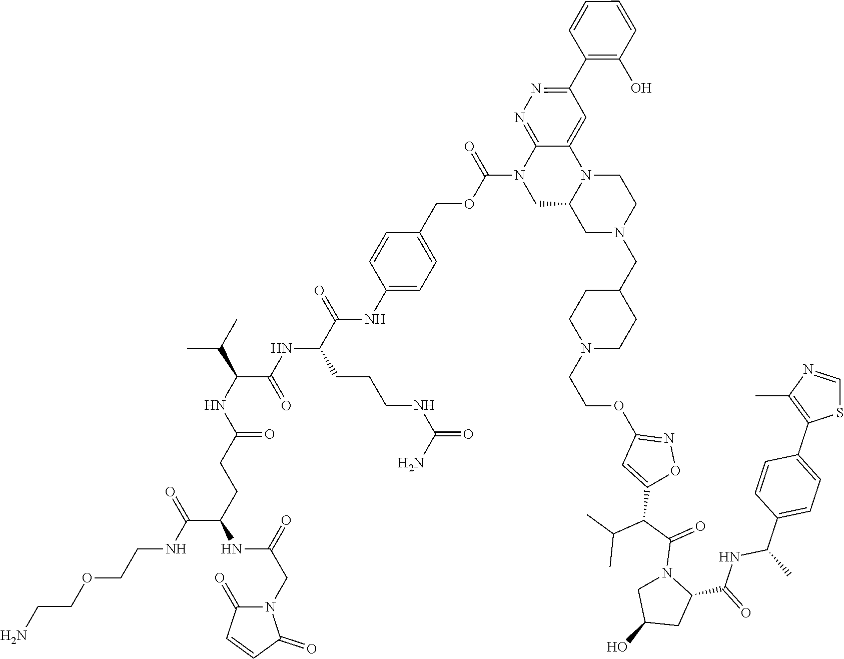 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CC[C@@H](NC(=O)CN7C(=O)C=CC7=O)C(=O)NCCOCCN)C(C)C)cc6)C[C@H]5C4)CC3)no2)C(C)C)cc1] |
| 2.54 | 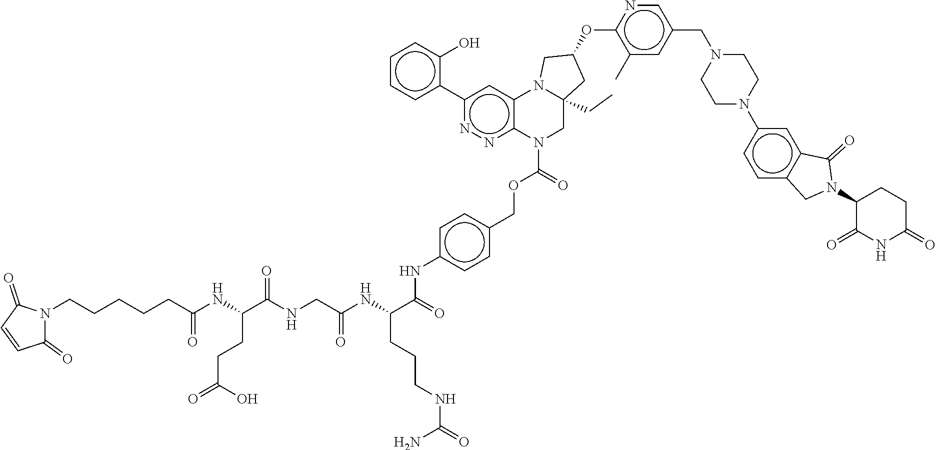 |
| 2.55 | 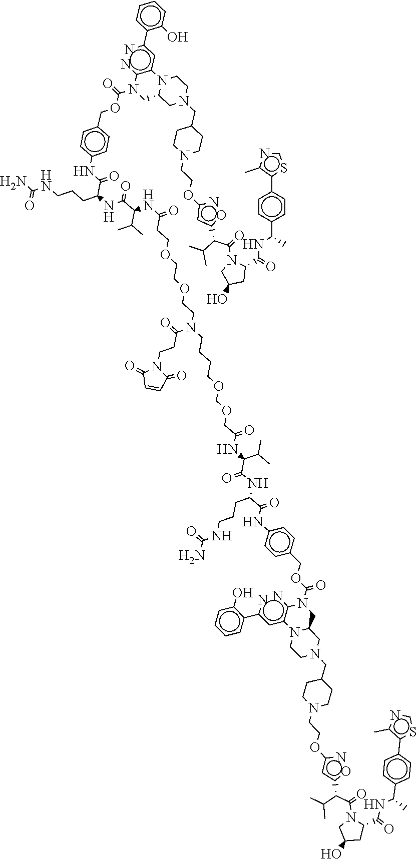 |
| 2.56 | 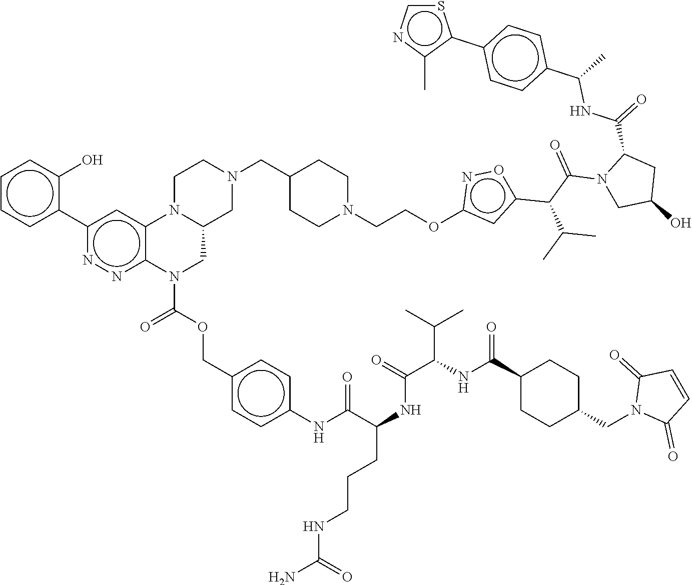 |
| 2.57 | 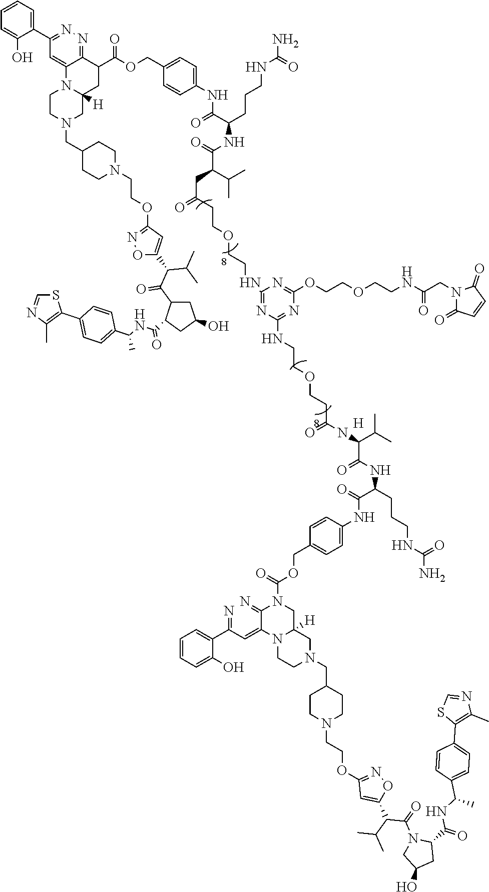 |
| 2.58 | 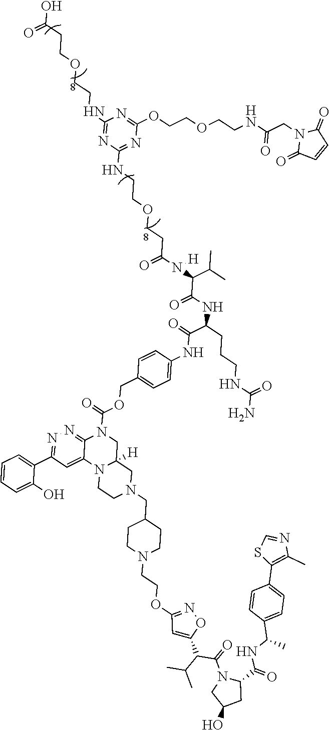 |
| 2.59 | 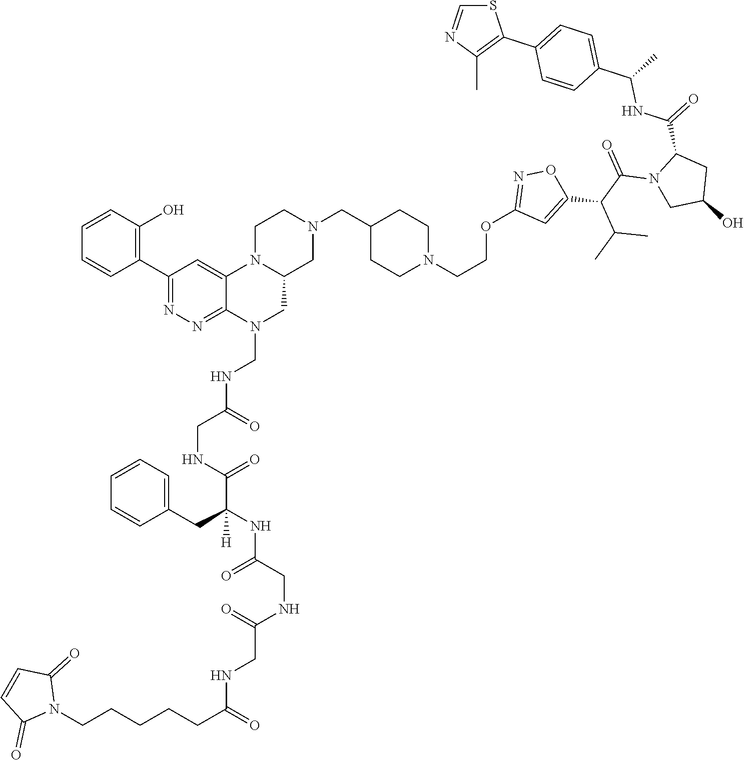 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(CNC(=O)CNC(=O)[C@H](Cc6ccccc6)NC(=O)CNC(=O)CNC(=O)CCCCCN6C(=O)C=CC6=O)C[C@H]5C4)CC3)no2)C(C)C)cc1] |
| 2.60 | 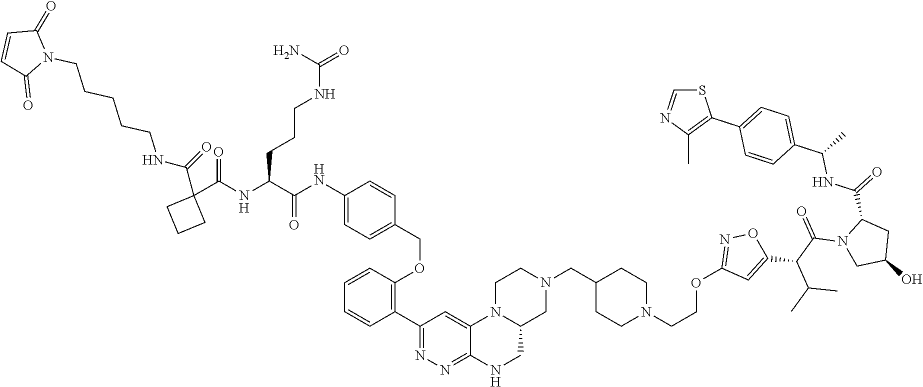 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7OCc7ccc(NC(=O)[C@H](CCCNC(N)=O)NC(=O)C8(C(=O)NCCCCCN9C(=O)C=CC9=O)CCC8)cc7)nnc6NC[C@H]5C4)CC3)no2)C(C)C)cc1] |
| 2.61 | 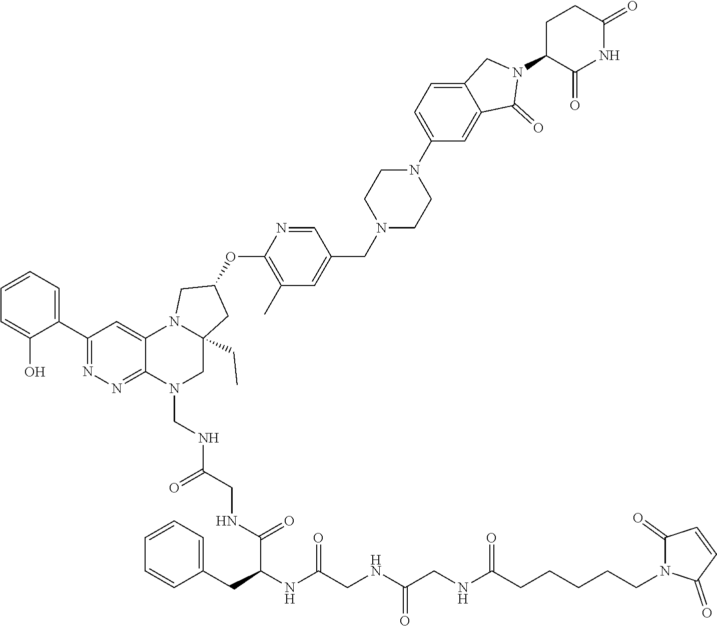 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3O)nnc1N(CNC(=O)CNC(=O)[C@H](Cc1ccccc1)NC(=O)CNC(=O)CNC(=O)CCCCCN1C(=O)C=CC1=O)C2] |
| 2.62 | 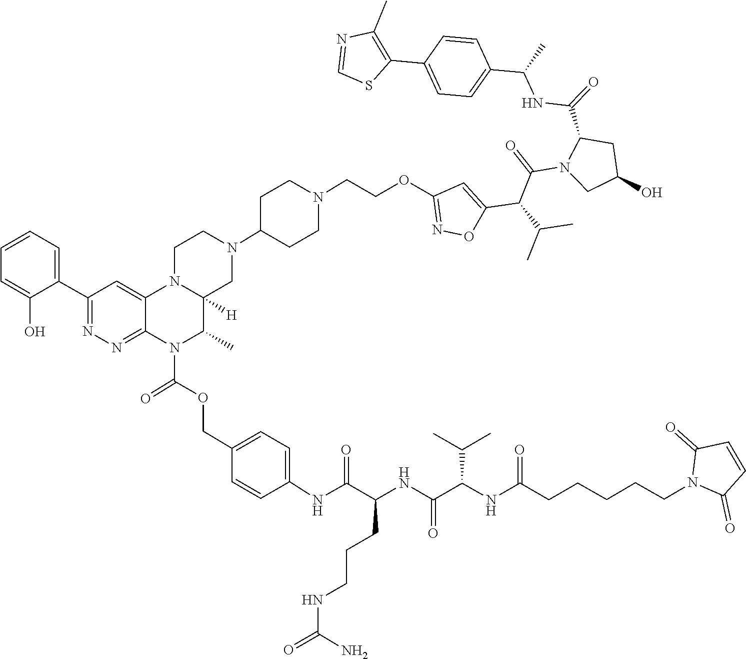 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C=CC7=O)C(C)C)cc6)[C@@H](C)[C@@H]5C4)CC3)no2)C(C)C)cc1] |
| 2.63 | 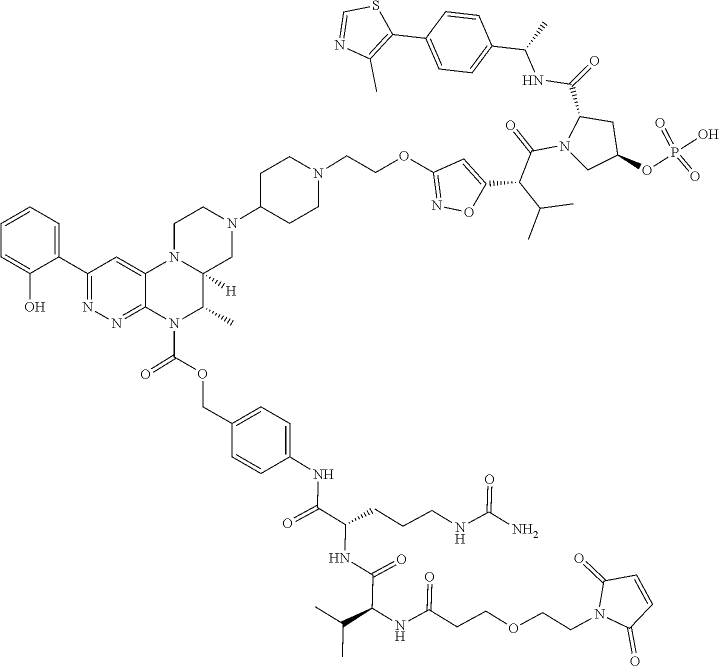 |
| 2.64 | 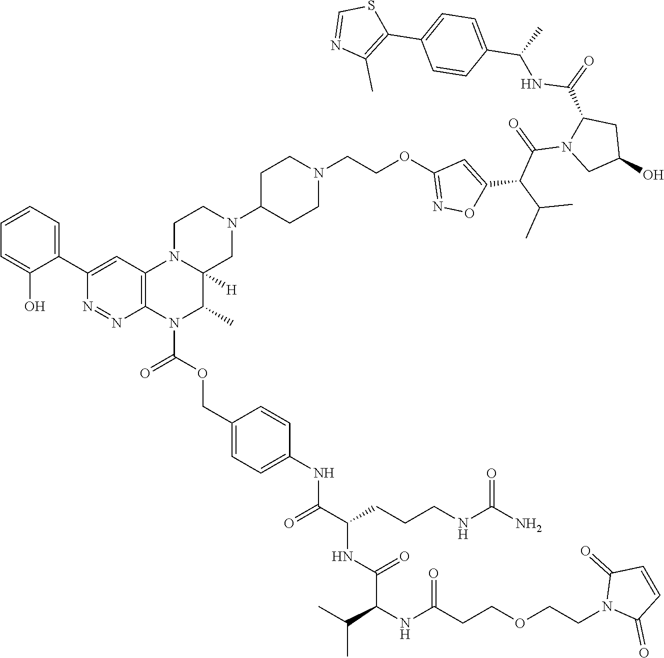 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCOCCN7C(=O)C=CC7=O)C(C)C)cc6)[C@@H](C)[C@@H]5C4)CC3)no2)C(C)C)cc1] |
| 2.65 | 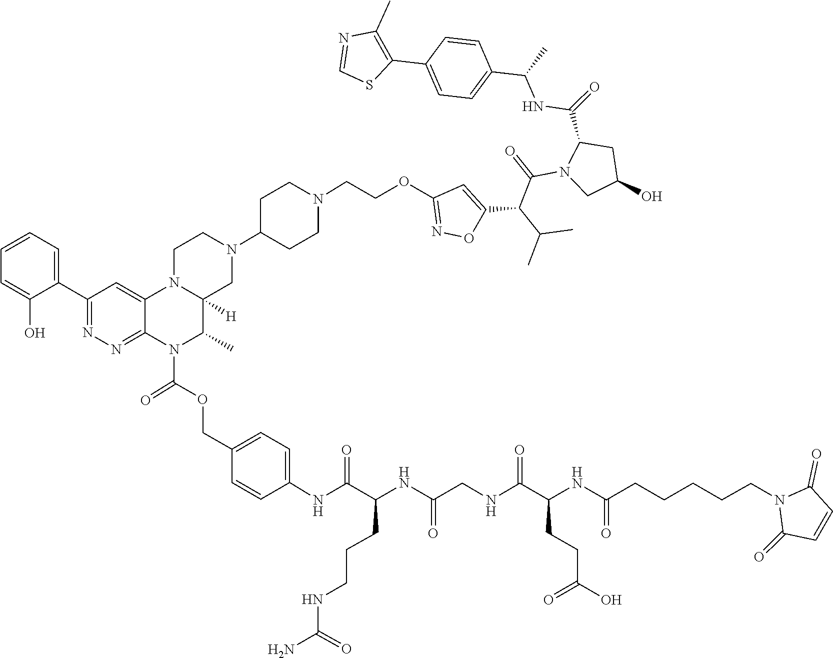 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)CNC(=O)[C@H](CCC(=O)O)NC(=O)CCCCCN7C(=O)C=CC7=O)cc6)[C@@H](C)[C@@H]5C4)CC3)no2)C(C)C)cc1] |
| 2.66 | 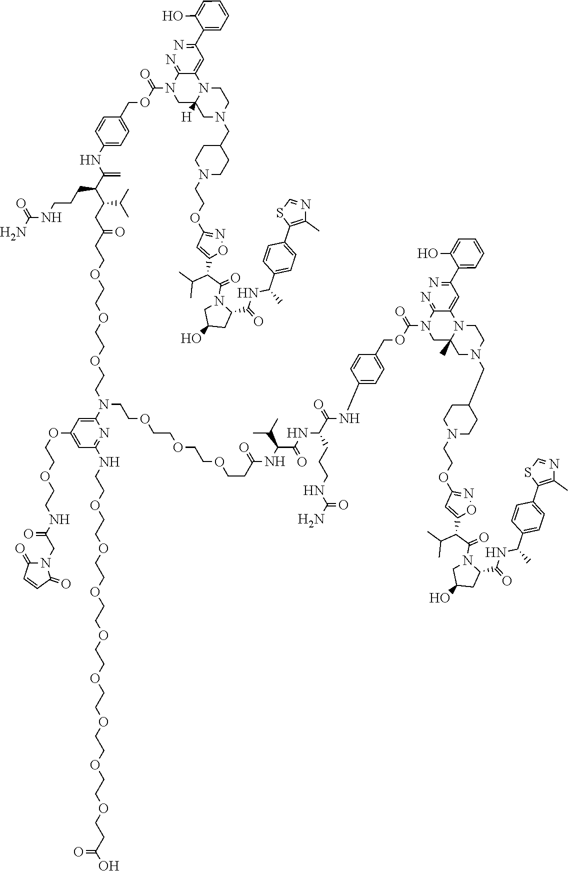 [SMILES: C=C(Nc1ccc(COC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)cc1)[C@H](CCCNC(N)=O)[C@@H](CC(=O)CCOCCOCCOCCN(CCOCCOCCOCCC(=O)N[C@H](C(=O)N[C@@H](CCCNC(N)=O)C(=O)Nc1ccc(COC(=O)N2C[C@@]3(C)CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)cc1)C(C)C)c1cc(OCCOCCNC(=O)CN2C(=O)C=CC2=O)cc(NCCOCCOCCOCCOCCOCCOCCOCCOCCC(=O)O)n1)C(C)C] |
| 2.67 | 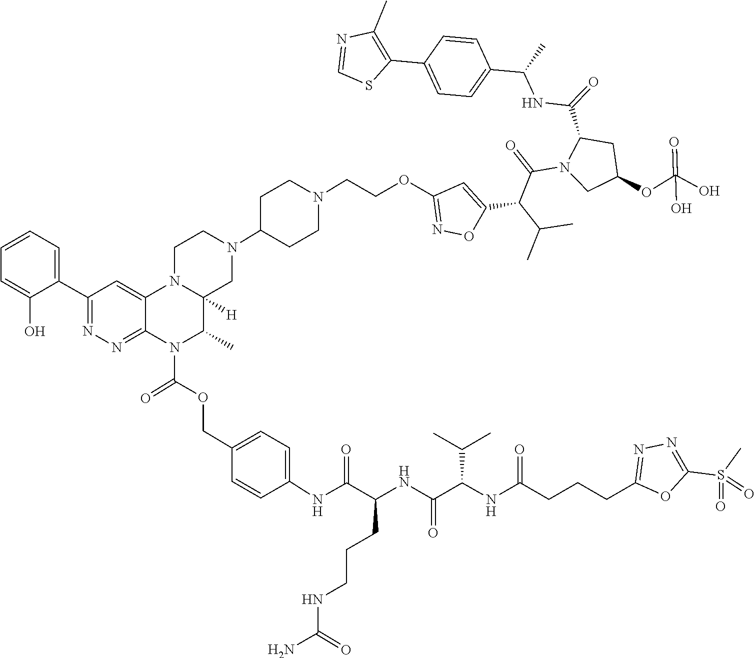 |
| 2.68 | 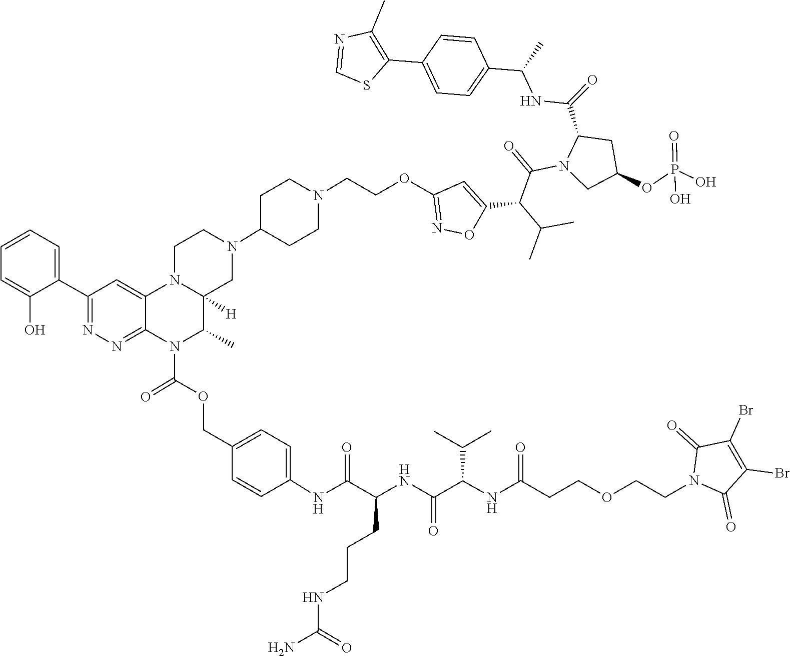 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCOCCN7C(=O)C(Br)=C(Br)C7=O)C(C)C)cc6)[C@@H](C)[C@@H]5C4)CC3)no2)C(C)C)cc1] |
| 2.69 | 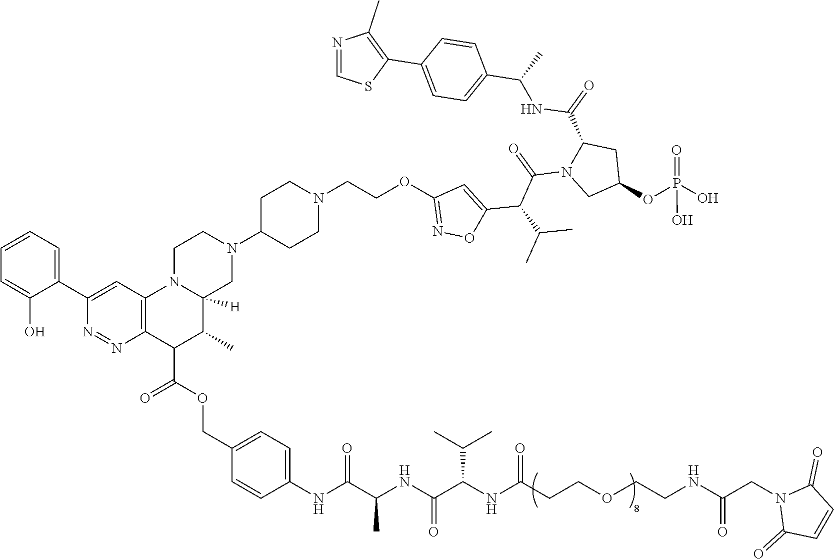 |
| 2.70 | 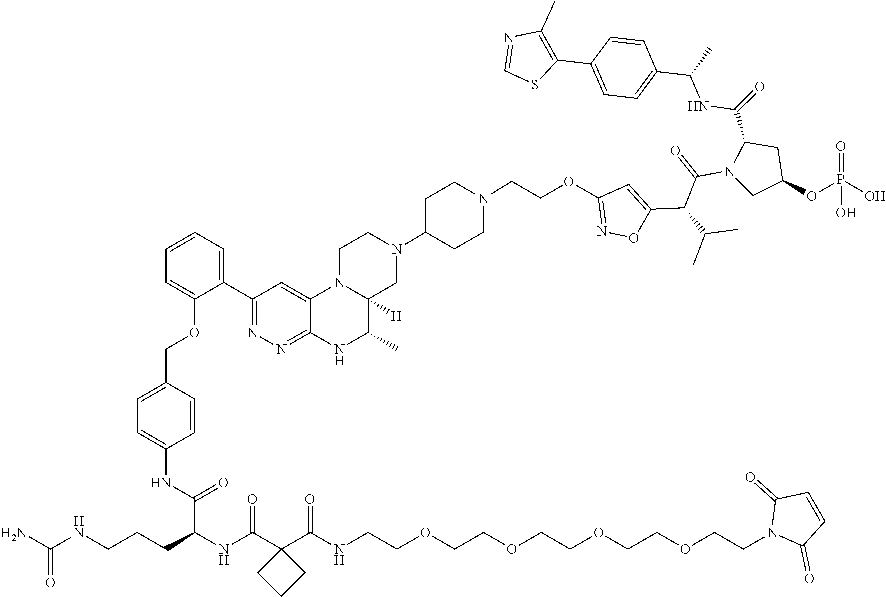 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7OCc7ccc(NC(=O)[C@H](CCCNC(N)=O)NC(=O)C8(C(=O)NCCOCCOCCOCCOCCN9C(=O)C=CC9=O)CCC8)cc7)nnc6N[C@@H](C)[C@@H]5C4)CC3)no2)C(C)C)cc1] |
| 2.71 | 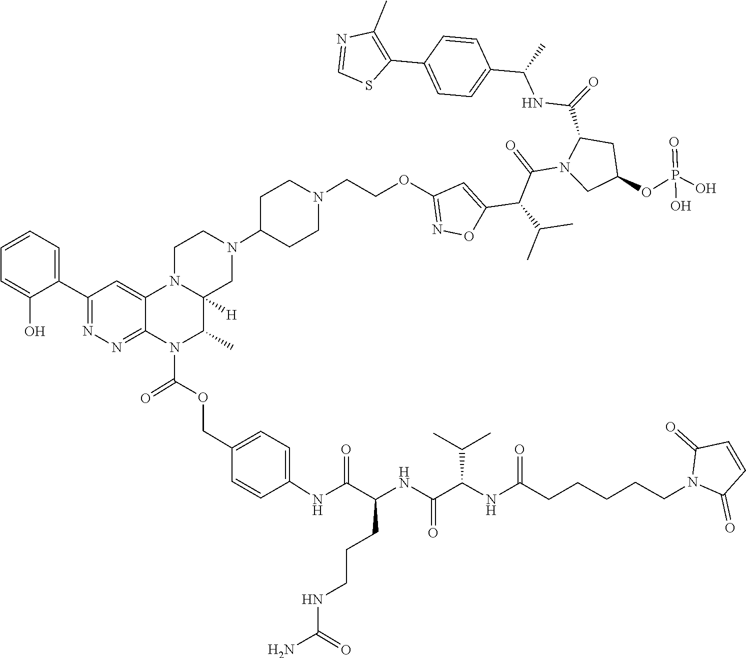 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C=CC7=O)C(C)C)cc6)[C@@H](C)[C@@H]5C4)CC3)no2)C(C)C)cc1] |
| 2.72 | 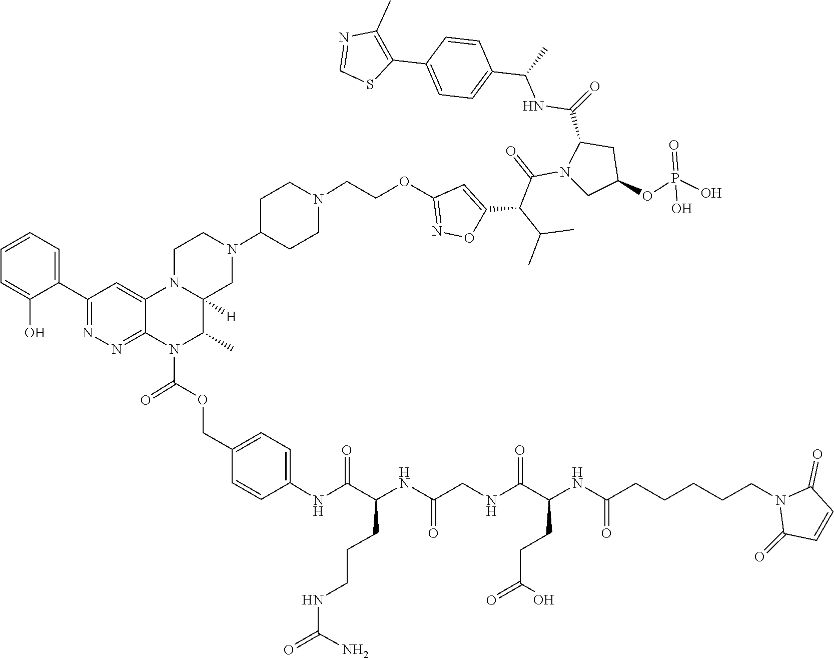 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)CNC(=O)[C@H](CCC(=O)O)NC(=O)CCCCCN7C(=O)C=CC7=O)cc6)[C@@H](C)[C@@H]5C4)CC3)no2)C(C)C)cc1] |
| 2.73 | 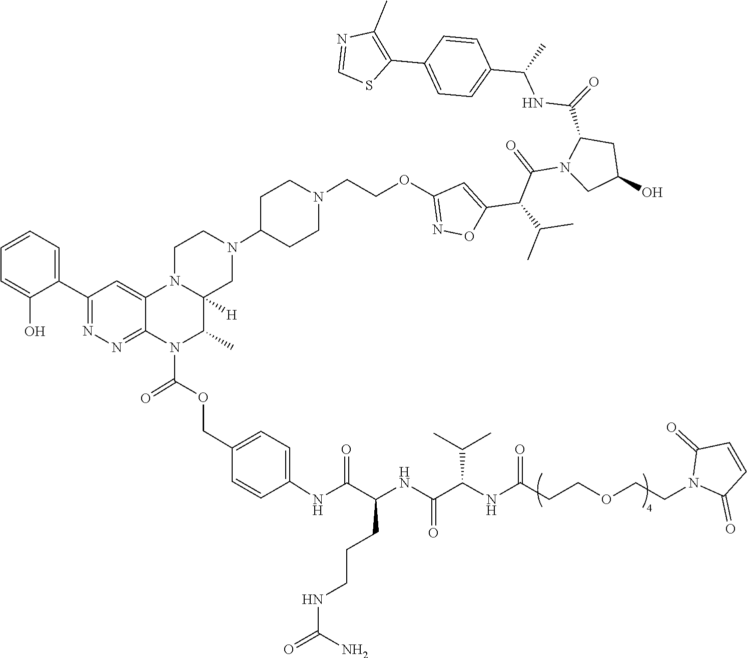 |
| 2.74 | 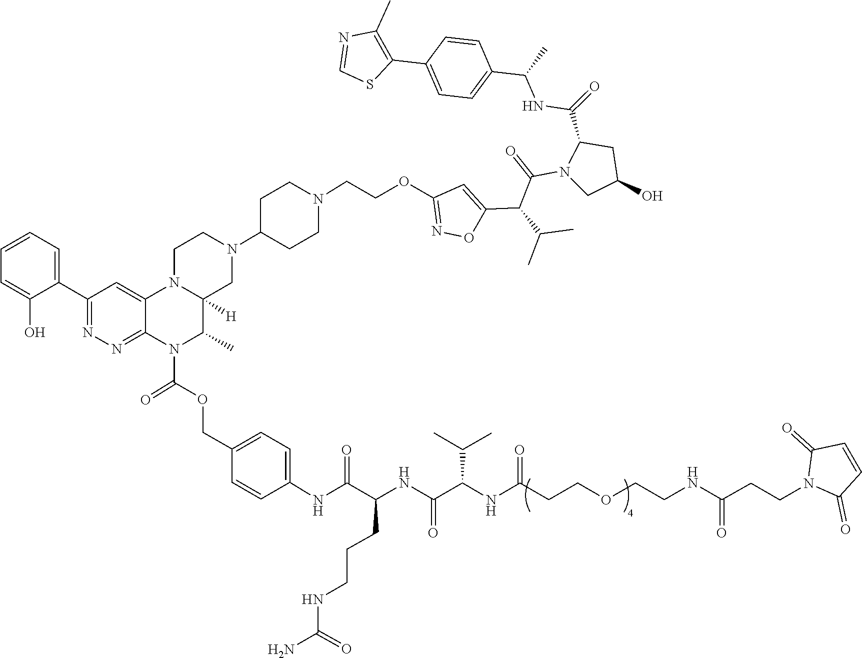 |
| 2.76 | 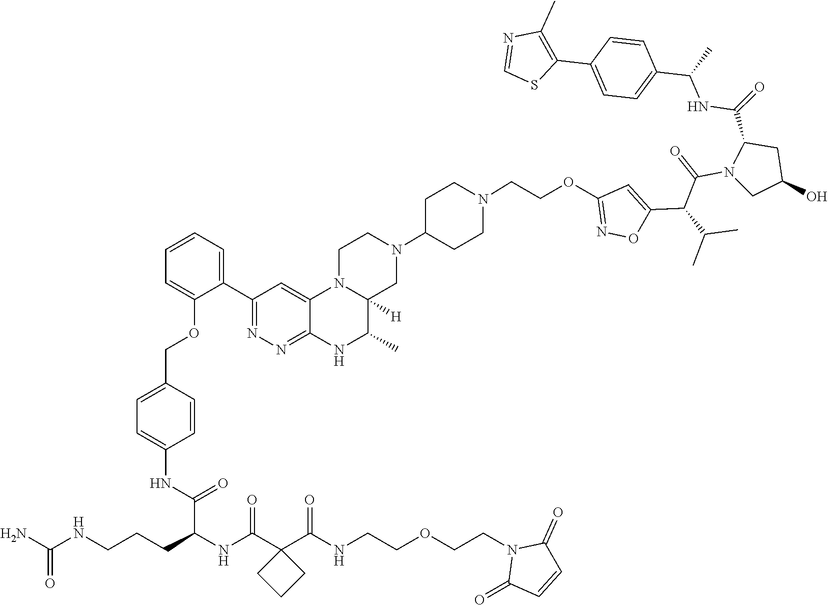 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7OCc7ccc(NC(=O)[C@H](CCCNC(N)=O)NC(=O)C8(C(=O)NCCOCCN9C(=O)C=CC9=O)CCC8)cc7)nnc6N[C@@H](C)[C@@H]5C4)CC3)no2)C(C)C)cc1] |
| 2.77 | 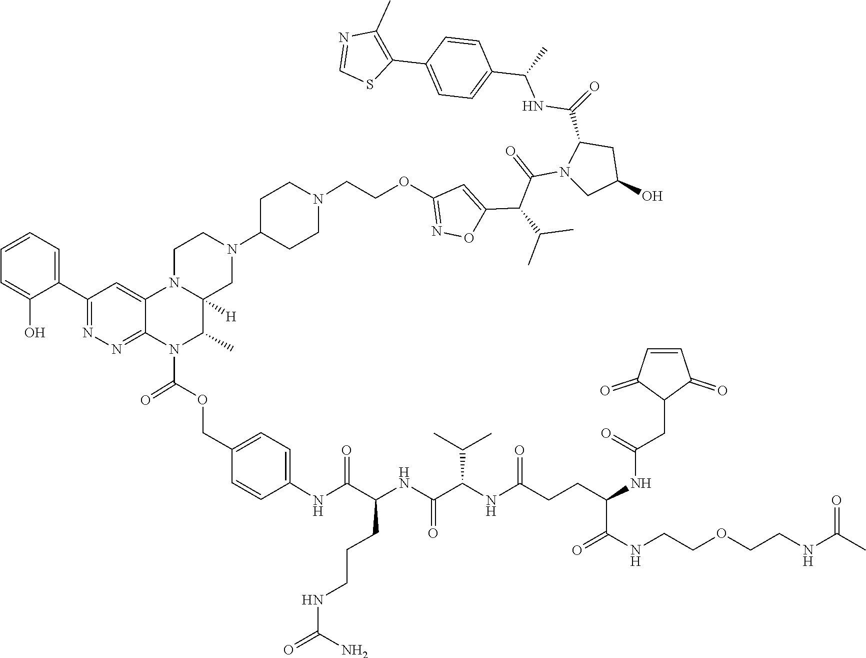 [SMILES: CC(=O)NCCOCCNC(=O)[C@@H](CCC(=O)N[C@H](C(=O)N[C@@H](CCCNC(N)=O)C(=O)Nc1ccc(COC(=O)N2c3nnc(-c4ccccc4O)cc3N3CCN(C4CCN(CCOc5cc([C@H](C(=O)N6C[C@H](O)C[C@H]6C(=O)N[C@@H](C)c6ccc(-c7scnc7C)cc6)C(C)C)on5)CC4)C[C@H]3[C@@H]2C)cc1)C(C)C)NC(=O)CC1C(=O)C=CC1=O] |
| 2.78 | 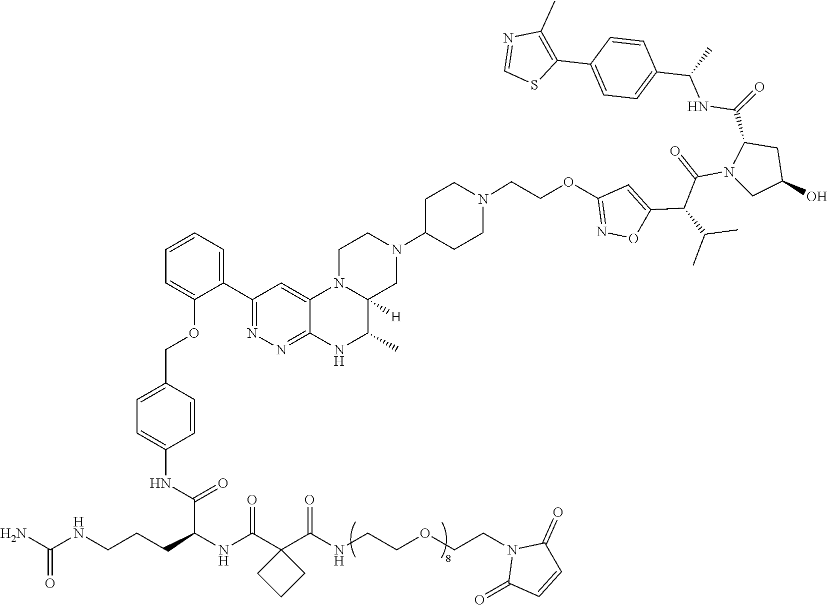 |
| 2.79 | 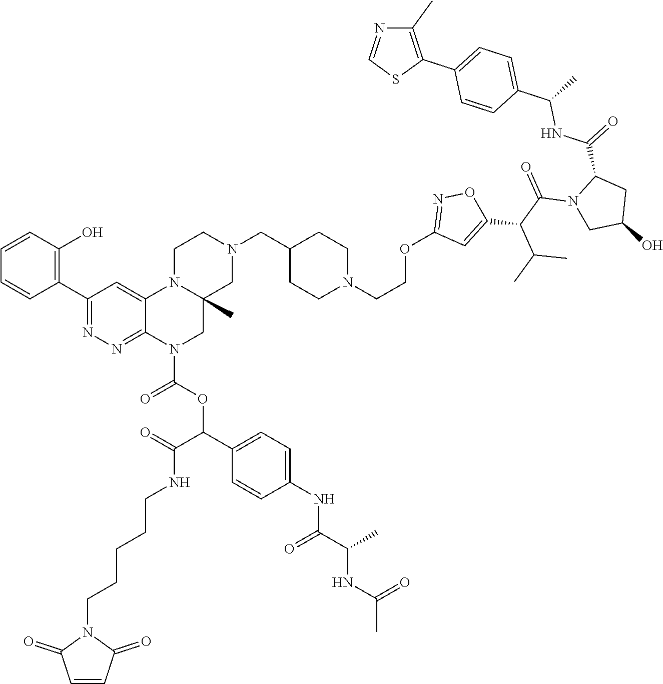 [SMILES: CC(=O)N[C@@H](C)C(=O)Nc1ccc(C(OC(=O)N2C[C@@]3(C)CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)C(=O)NCCCCCN2C(=O)C=CC2=O)cc1] |
| 2.80 | 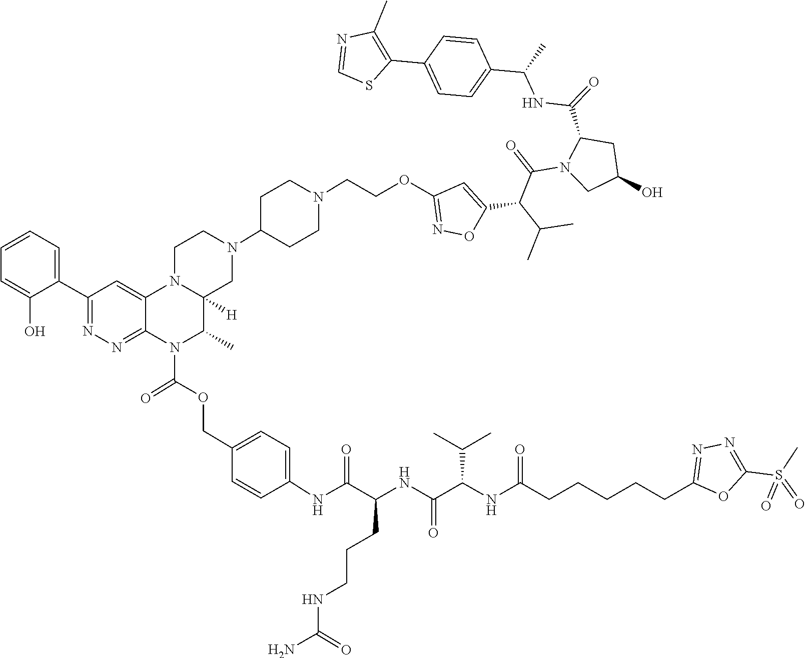 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCCCCc7nnc(S(C)(=O)=O)o7)C(C)C)cc6)[C@@H](C)[C@@H]5C4)CC3)no2)C(C)C)cc1] |
| 2.81 | 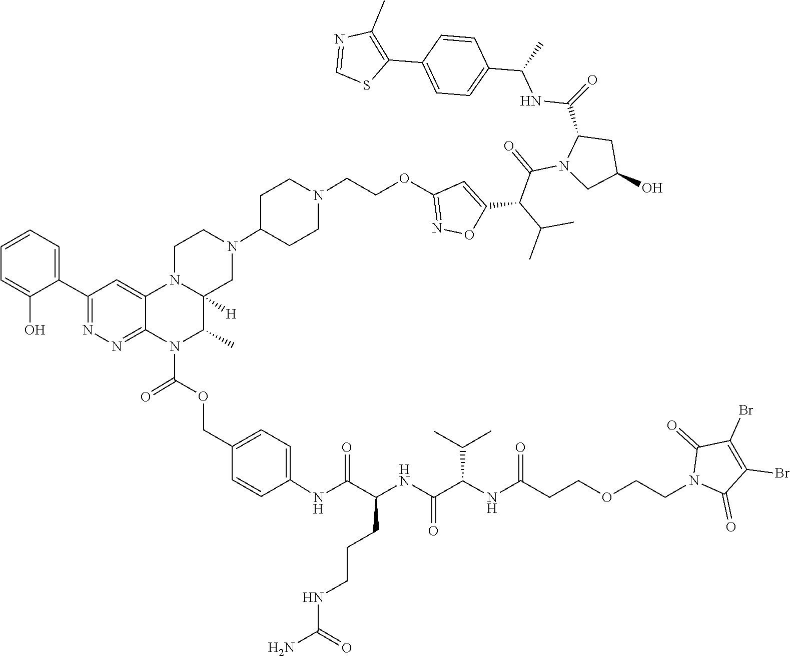 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCOCCN7C(=O)C(Br)=C(Br)C7=O)C(C)C)cc6)[C@@H](C)[C@@H]5C4)CC3)no2)C(C)C)cc1] |
| 2.82 | 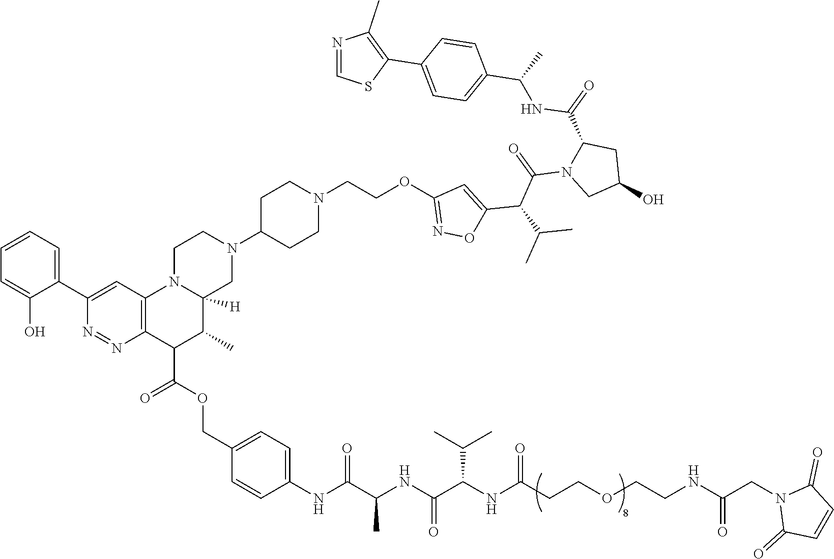 |
| 2.83 | 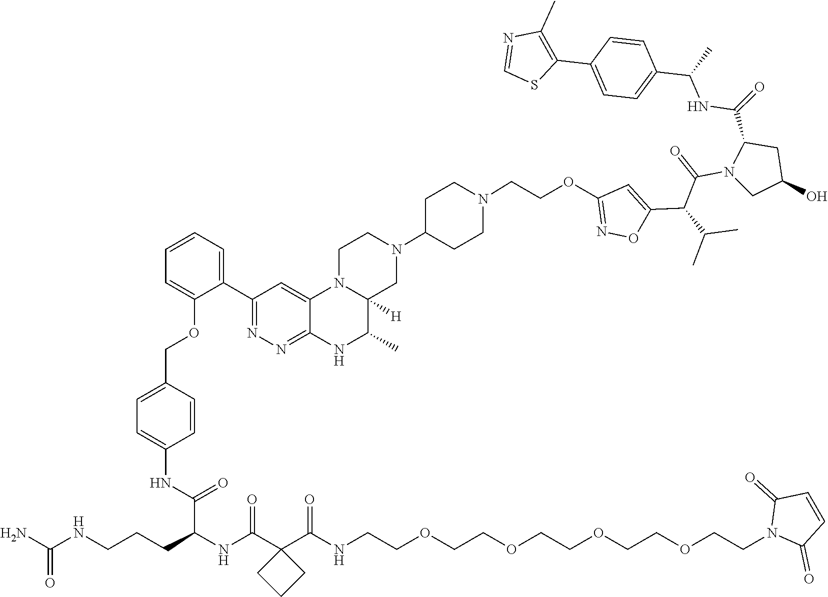 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7OCc7ccc(NC(=O)[C@H](CCCNC(N)=O)NC(=O)C8(C(=O)NCCOCCOCCOCCOCCN9C(=O)C=CC9=O)CCC8)cc7)nnc6N[C@@H](C)[C@@H]5C4)CC3)no2)C(C)C)cc1] |
| 2.84 | 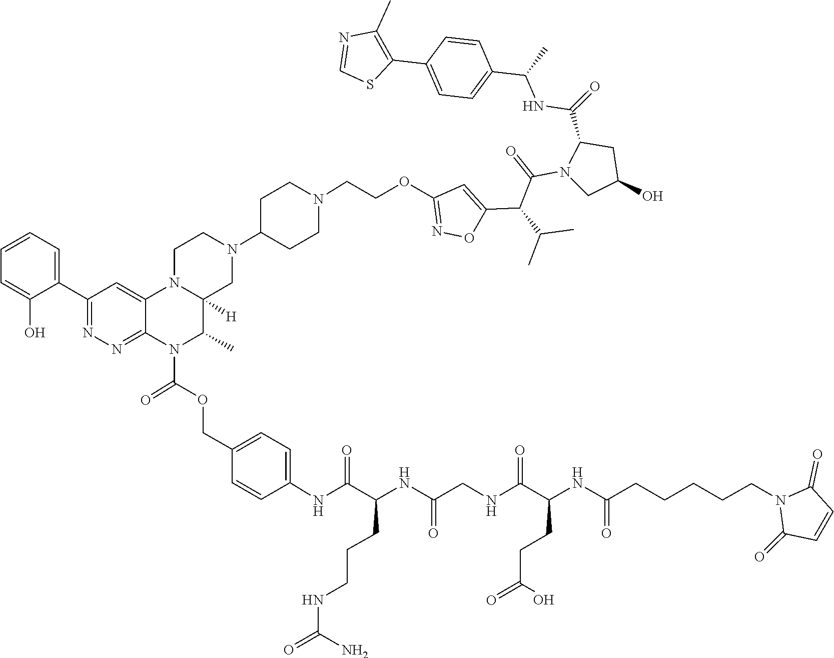 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)CNC(=O)[C@H](CCC(=O)O)NC(=O)CCCCCN7C(=O)C=CC7=O)cc6)[C@@H](C)[C@@H]5C4)CC3)no2)C(C)C)cc1] |
| 2.85 | 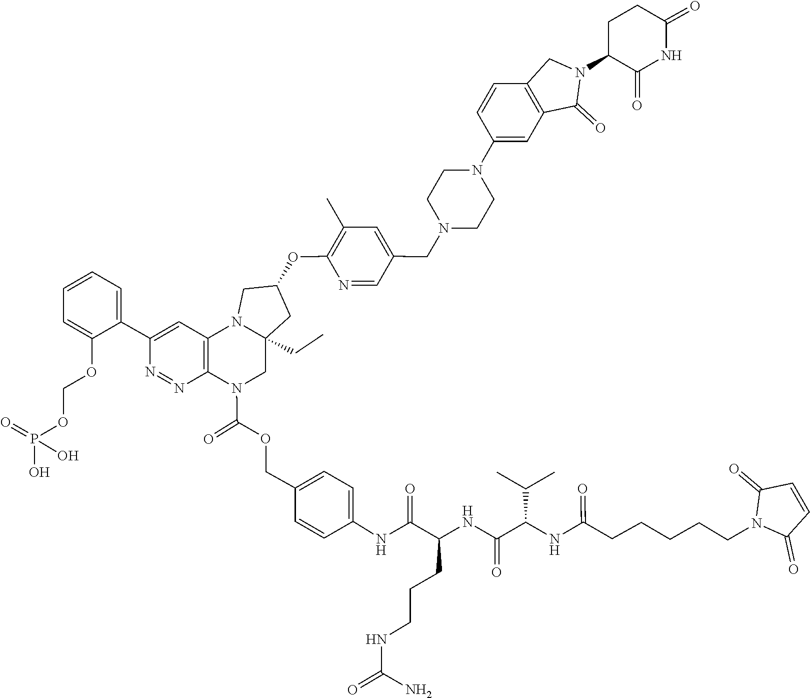 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3OCOP(=O)(O)O)nnc1N(C(=O)OCc1ccc(NC(=O)[C@H](CCCNC(N)=O)NC(=O)[C@@H](NC(=O)CCCCCN3C(=O)C=CC3=O)C(C)C)cc1)C2] |

### III. Pharmaceutical Compositions

In one aspect, provided herein is a pharmaceutical composition comprising one or more Antibody Drug Conjugate compounds as described herein. In some embodiments, the pharmaceutical compositions described herein are for use in the method of any of the embodiments described herein.

In some embodiments, the pharmaceutical composition comprises a therapeutically effective amount of one or more of the Antibody Drug Conjugate compounds of the present disclosure as the active ingredient, or a pharmaceutically acceptable salt, ester, prodrug, solvate, hydrate or derivative thereof.

In some embodiments, a pharmaceutical composition comprises a number of antibody-drug conjugates. In some embodiments, the antibody-drug conjugates of the pharmaceutical composition substantially differ from each other only in their drug loading and/or the distribution of drug linker moieties on the antibody. In some embodiments, the number of drug linker moieties on the antibody is the same for each antibody-drug conjugate of the pharmaceutical composition. In some embodiments, the distribution of drug linker moieties on the antibody is the same for each antibody-drug conjugate of the pharmaceutical composition. In some embodiments, the pharmaceutical composition is heterogenous. In some embodiments, the drug loading on each antibody of the pharmaceutical composition is an integer that ranges from 1 to 14.

In some embodiments, the average drug loading of the antibody-drug conjugates of the pharmaceutical composition is a number ranging from about 1 to about 14. In some embodiments, the average drug loading of the antibody-drug conjugates of the pharmaceutical composition is a number ranging from about 1 to about 8. In some embodiments, a small percentage of antibodies may be conjugated to zero drug linker moieties. In some embodiments, the average drug loading or the average number of drug linker moieties conjugated to an antibody determines the amount of the degrader compound that is delivered. In some embodiments, the average drug loading of the antibody-drug conjugates of the pharmaceutical composition is 1, about 1, 2, about 2, 3, about 3, 4, about 4, 5, about 5, 6, about 6, 7, about 7, 8, about 8, 9, about 9, 10, about 10, 11, about 11, 12, about 12, 13, about 13, 14, or about 14. In some embodiments, the average drug loading of the antibody-drug conjugates of the pharmaceutical composition is about 1 to about 8, about 2 to about 8, about 1 to about 4, about 2 to about 4, or about 4 to about 8. In some embodiments, the average number of drug linker moieties conjugated to an antibody is 1, about 1, 2, about 2, 3, about 3, 4, about 4, 5, about 5, 6, about 6, 7, about 7, 8, or about 8. In some embodiments, the average number of drug linker moieties conjugated to an antibody is about 1 to about 8, about 2 to about 8, about 1 to about 4, about 2 to about 4, or about 4 to about 8.

The average drug loading or average number of drug linker moieties conjugated to an antibody in a composition of antibody-drug conjugates, as well as the distribution of the antibody-drug conjugates in terms of subscript z, may be determined by conventional methods such as mass spectrometry, ELISA assay, or HPLC (e.g., HIC). Homogenous pharmaceutical compositions of antibody-drug conjugates may be separated, purified, and characterized using conventional methods such as reverse phase HPLC or electrophoresis.

A pharmaceutical composition of the invention typically contains an active ingredient (e.g., one or more Antibody Drug Conjugate compounds) described herein, or a pharmaceutically acceptable salt thereof, and one or more pharmaceutically acceptable excipients or carriers. In some embodiments, the one or more pharmaceutically acceptable excipients or carriers includes, but is not limited to, inert solid diluents and fillers, diluents, sterile aqueous solutions, various organic solvents, permeation enhancers, solubilizers and adjuvants. In some embodiments, the pharmaceutical composition comprising a number of antibody-drug conjugates as described herein is in the form of a liquid. In some embodiments, the pharmaceutical composition comprising a number of antibody-drug conjugates as described herein is in the form of a solid. In some embodiments, the pharmaceutical composition comprising a number of antibody-drug conjugates as described herein is in the form of a lyophilized powder. In some embodiments, the pharmaceutical composition comprising a number of antibody-drug conjugates as described herein is in the form of a solution or suspension provided by a lyophilized powder that has been reconstituted.

In some embodiments, the pharmaceutical composition comprising one or more Antibody Drug Conjugate compounds as described herein further comprises one or more other agents, which are also typically administered in the form of pharmaceutical compositions. For example, in some embodiments, the one or more Antibody Drug Conjugate compounds and the one or more other agents are formulated into the same pharmaceutical formulation for use at the same time. In other embodiments, the pharmaceutical composition comprising one or more Antibody Drug Conjugate compounds and the one or more other agents are formulated into separate pharmaceutical compositions for use in combination separately or at the same time.

In some embodiments, the concentration of one or more conjugates provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, ,10% 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more conjugates of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more conjugates of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more conjugates of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

The conjugates according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. In some embodiments, the dosage administered to a patient is about 0.01 mg/kg to about 100 mg/kg of the subject's body weight, or is about 0.1 mg/kg to about 25 mg/kg of the subject's body weight. The exact dosage will depend upon the route of administration, the form in which the conjugate is administered, the subject to be treated, the body weight of the subject to be treated, and the preference and experience of the attending physician.

### IV. Methods of Administration

A pharmaceutical composition or conjugate of the present disclosure can be administered by a variety of methods known in the art. The route and/or mode of administration may vary depending upon the desired results. In some embodiments, the pharmaceutical formulation is solubilized and administered via any route capable of delivering the pharmaceutical composition or conjugate to the desired site. Potentially effective routes of administration include, but are not limited to, parenteral (e.g., intravenous, subcutaneous, intrasternal), intraperitoneal, intramuscular, intratumor, intradermal, intraorgan, orthotopic, and the like. In some embodiments, the administration is intravenous, subcutaneous, intraperitoneal, or intramuscular. The pharmaceutically acceptable carrier should be suitable for the route of administration, e.g., intravenous or subcutaneous administration (e.g., by injection or infusion). In some embodiments, the administration is by infusion. In some embodiments, the administration is by a bolus injection.

### Pharmaceutical Compositions for Injection

In some embodiments, the invention provides a pharmaceutical composition for injection comprising an Antibody Drug Conjugate compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

In some embodiments, the pharmaceutical composition comprising a number of antibody-drug conjugates as described herein is in the form of a liquid. In some embodiments, the pharmaceutical composition comprising a number of antibody-drug conjugates as described herein is in the form of a solid. In some embodiments, the pharmaceutical composition comprising a number of antibody-drug conjugates as described herein is in the form of a lyophilized powder. In some embodiments, the pharmaceutical composition comprising a number of antibody-drug conjugates as described herein is in the form of a solution or suspension provided by a lyophilized powder that has been reconstituted.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the conjugate of the present invention in the required amount in the appropriate solvent with various other ingredients as enumerated above, as required, followed by filtered sterilization. In the case of sterile powders for the preparation of sterile injectable solutions, certain desirable methods of preparation are vacuum-drying and freeze- drying techniques which yield a powder of the active ingredient plus any additional desired ingredient from a previously sterile-filtered solution thereof.

### Other Pharmaceutical Compositions

A pharmaceutical composition or conjugate of the present disclosure can be administered by any suitable route or mode. In some embodiments, the pharmaceutical composition comprising an Antibody Drug Conjugate compound, or a pharmaceutically acceptable salt thereof, of the present invention are administered parenterally. For example, parenteral administration includes intravenous, subcutaneous, intradermal, intramuscular, intraperitoneal, intrathecal, and intrathecal injection or infusion. In some embodiments, the conjugates or pharmaceutical composition comprising an Antibody Drug Conjugate compound, or a pharmaceutically acceptable salt thereof, of the present invention is administered by intravenous injection.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the conjugates or pharmaceutical composition of the present invention can be affected by any method that enables delivery of the conjugates or compounds to the site of action. These methods include parenteral injection (including intravenous, intraarterial, subcutaneous, intramuscular, intravascular, intraperitoneal or infusion).

In some embodiments, the conjugates of the present invention are administered by intravenous injection.

### V. Methods of Use

The antibody-drug conjugates described herein may be used for treating a cancer in a patient, for treating or preventing a disease or disorder in which SMARCA2 and/or SMARCA4 plays a role, or for treating or preventing a disease or disorder in which SWI/SNF mutations play a role. In some embodiments, the antibody-drug conjugates described herein deliver a drug to a tumor or cancer cell, wherein the drug inhibits or degrades SMARCA2 and/or SMARCA4. In some embodiments, the antibody-drug conjugate is internalized through endocytosis after binding or associating with an antigen that is associated with a tumor cell or a cancer cell. In some embodiments, a degrader drug (D) is released from the antibody-drug conjugate after the antibody-drug conjugate is internalized by a cell. In some embodiments, a degrader drug (D) penetrates a tumor or cancer cell after being released from the antibody-drug conjugate outside the cell.

In some embodiments, the cancers that are most effectively treated using the antibody-drug conjugates described herein are determined by the specificity of the antibody.

In some embodiments, the cancer is characterized by uncontrolled cell growth. In some embodiments, the cancer is a tumor, metastasis, or other disease or disorder.

In some embodiments, provided is a method for treating cancer, comprising administering an effective amount of an antibody-drug conjugate described herein to a patient in need thereof. In some embodiments, the method further comprises administering a chemotherapeutic agent. In some embodiments, the cancer has been found to be resistant to the chemotherapeutic agent. In some embodiments, the cancer has not been found to be resistant to the chemotherapeutic agent. In some embodiments, the patient has undergone surgery and/or has received an additional treatment. In some embodiments, the patient receives radiation treatment in addition to being administered a pharmaceutical composition comprising a number of antibody-drug conjugates as described herein. In some embodiments, the additional treatment comprising a chemotherapeutic agent or radiation therapy is administered prior to administration of a pharmaceutical composition comprising a number of antibody-drug conjugates as described herein. In some embodiments, the additional treatment comprising a chemotherapeutic agent or radiation therapy is administered subsequent to administration of a pharmaceutical composition comprising a number of antibody-drug conjugates as described herein.

In some embodiments, methods of treatment comprising administering an antibody-drug conjugate or a composition thereof described herein to a subject in need thereof are an alternative to chemotherapy or radiation therapy. In some of these embodiments, chemotherapy or radiation therapy results in unacceptable side effects. In some embodiments, the subject is optionally treated with another cancer treatment. In some embodiments, the subject is optionally treated with surgery, radiation therapy, or chemotherapy.

The method typically comprises administering to a subject a therapeutically effective amount of a pharmaceutical composition or conjugate of the invention. The therapeutically effective amount of the subject combination of conjugates may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular conjugates chosen, the dosing regimen to be followed, whether it is administered in combination with other compounds, timing of administration, the tissue to which it is administered, and the physical delivery system in which it is carried.

In certain embodiment, target proteins are SMARCA2, SMARCA4 and/or PB1.

In certain embodiment, target protein complex is SWI/SNF in a cell.

In certain embodiment, diseases or disorders dependent on SMARCA2 or SMARCA4 include cancers.

In some embodiments, the present conjugates treat cancer. In some embodiments, the cancer is prostate cancer. In some embodiments, the cancer is acute myeloid Leukemia (AML). In some embodiments, the cancer is liver cancer. In some embodiments, the cancer is melanoma. In some embodiments, the cancer is multiple myeloma (MM). In some embodiments, the cancer is breast cancer. In some embodiments, the breast cancer is triple-negative breast cancer. In some embodiments, the breast cancer is estrogen receptor positive (ER+) and human epidermal growth factor receptor 2 negative (HER2−) breast cancer. In some embodiments, the cancer is lung cancer. In some embodiments, the cancer is colorectal cancer. In some embodiments, the cancer is ovarian cancer. In some embodiments, the cancer is gastric cancer. In some embodiments, the caner is bladder cancer. In some embodiments, the cancer is lymphoma.

In certain further embodiments, the cancer is a SMARCA2 and/or SMARAC4-dependent cancer. In some embodiments, the cancer harbors a SMARCA4 mutation.

Without being bound by theory, the SMARCA2/4 degrader payloads described herein may induce robust anti-proliferative and cytotoxic activity in prostate cancer cell lines, including reducing the expression of the androgen receptor (AR), AR splice variant (AR-V7), and ERG proteins.

In certain embodiment, diseases or disorders dependent on SWI/SNF complex include cancers.

Conjugates of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, conjugates of the disclosure, as well as pharmaceutical compositions comprising them, can be administered to treat any of the described diseases, alone or in combination with one or more other agents.

In other methods, the conjugates of the disclosure, as well as pharmaceutical compositions comprising them, can be administered in combination with agonists of nuclear receptors agents.

In other methods, the conjugates of the disclosure, as well as pharmaceutical compositions comprising them, can be administered in combination with antagonists of nuclear receptors agents.

In other methods, the conjugates of the disclosure, as well as pharmaceutical compositions comprising them, can be administered in combination with an anti-proliferative agent.

### VI. Methods of Preparation

### General Synthetic Procedures

The Antibody Drug Conjugates, degrader-linker compounds, and degrader compounds described herein may be prepared according to the following synthetic schemes and general synthetic procedures.

Degrader-linker compounds can be prepared from optionally protected compounds 1-1 as shown in Scheme I. Compounds 1-1 can be united with compounds 1-2 where LG¹ is a halogen (e.g., Cl or Br) or other leaving group (e.g., 4-nitropenol) under acylation standard conditions, such as in the presence of a base (e.g., 2,6-luditine), or under standard alkylation conditions, such as in the presence of a base (e.g., sodium hydride), to afford compounds 1-3. In some instances, subsequent optional global or selective deprotection can also afford degrader-linker compounds 1-3.

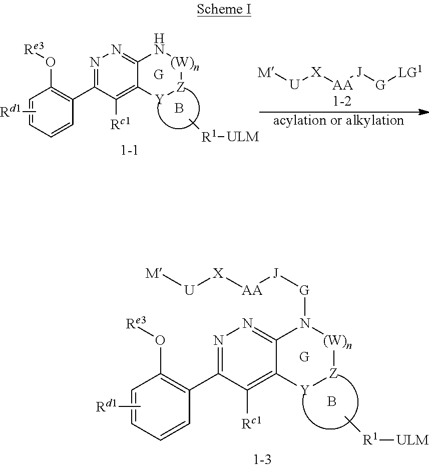

Degrader-linker compounds can be prepared from compounds as shown in Scheme II. Acylation of optionally protected compounds 1-1 with compounds 2-1 where PG¹ is a suitable amine protecting group (e.g., Boc, Fmoc, or Cbz) under standard acylation conditions, such as in the presence of a base (e.g., 2,6-luditine) or under standard alkylation conditions, such as in the presence of a base (e.g., sodium hydride), can afford compounds 2-2. Compounds 2-2 can be converted to compounds 2-3 under standard deprotection conditions, such as in the presence of an acid (e.g., HCl) or a base (e.g., DBU). Reaction of compounds 2-3 with compounds 2-4 where R^{ab} is H or succinimidyl group under standard amide bond formation conditions, such as in the presence of an amide coupling reagent (e.g., N,N′-cabonyldiimidazole or HATU) and a base (e.g., diisopropylethylamine) or in the presence of a mild base (e.g., NaHCO₃) can provide compounds 2-5. In some instances, subsequent optional global or selective deprotection can also afford degrader-linker compounds 2-5.

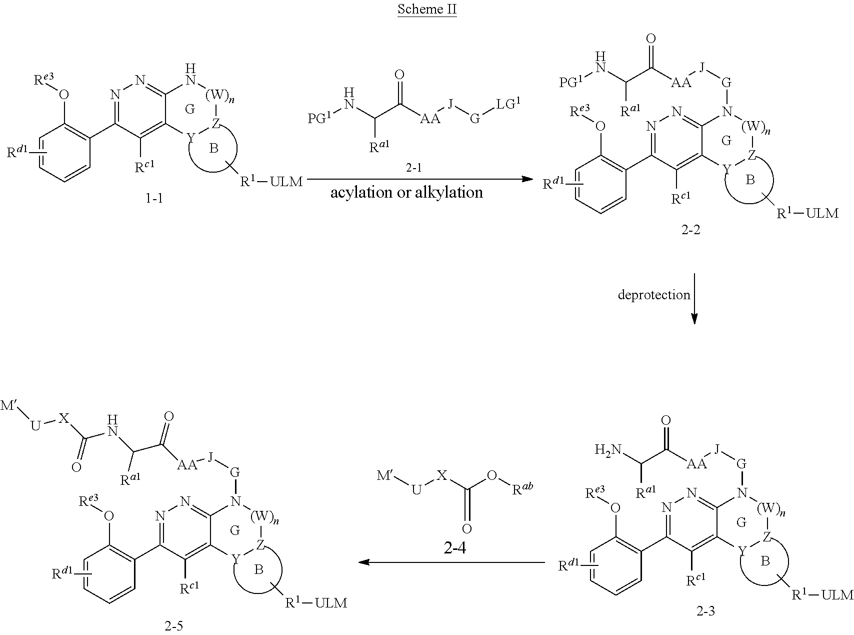

Intermediates for the synthesis of degrader-linker compounds can be prepared as shown in Scheme III. Deprotection of optionally protected compounds 3-1 where R^{aa} is C₁-C₈ alkyl or C₂-C₈ cycloalkyl, x is an integer from 0-6 under standard conditions, such as in the presence of an acid (e.g., PPTS) can afford compounds 3-2. Reaction of aldehydes 3-2 with amines 3-3 where y is an integer from 0-6 under standard reductive amination conditions (e.g., in the presence of a reducing agent, such as sodium triacetoxyborohydride or sodium cyanoborohydride, and optionally an acid, such as acetic acid, or a base, such as triethylamine) can provide compounds 2-2 where R¹ is -(A)_{q}-. In some instances, subsequent optional global or selective deprotection can also afford compounds 2-2.

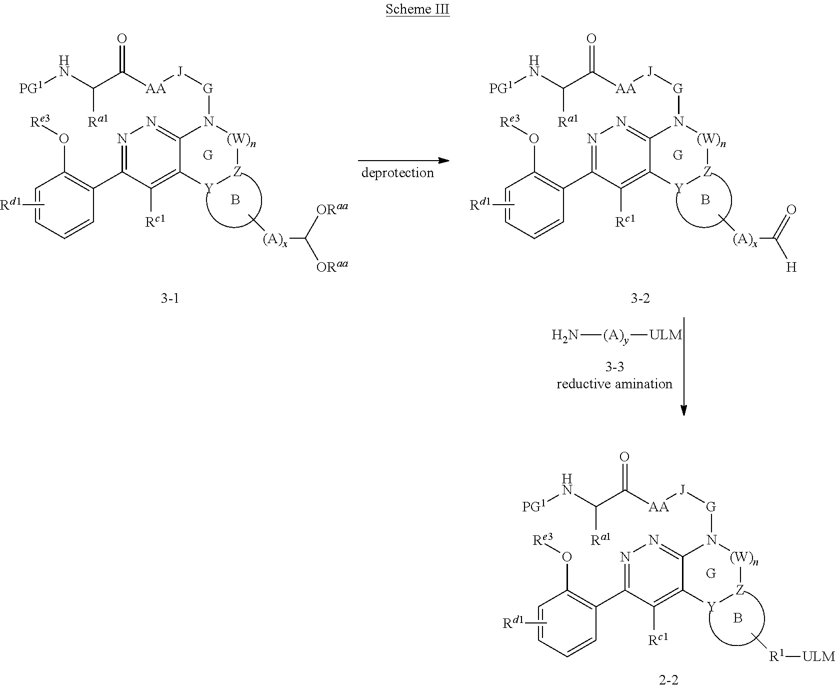

### EXAMPLES

The presently disclosed subject matter will be better understood by reference to the following Examples, which are provided as exemplary of the invention, and not by way of limitation.

### Example 1. Synthesis of Degrader Compounds

### 1A. Synthesis of (3S)-3-[5-[4-[[6-[[(4R,6S)-6-Ethyl-12-(2-hydroxyphenyl)-2,8,10,11-tetrazatricyclo[7.4.0.02,6]trideca-1(9),10,12-trien-4-yl]oxy]-5-methyl-3-pyridinyl]methyl]piperazin-1-yl]-3-oxo-1H-isoindol-2-yl]piperidine-2,6-dione

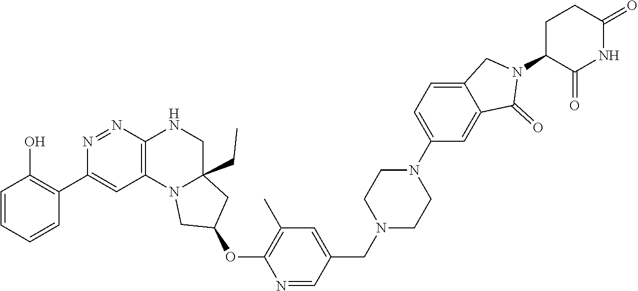 [SMILES: CC[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)cc1C)C2]

### Step 1. 1-(tert-Butyl) 2-methyl (4R)-4-(benzyloxy)-2-ethylpyrrolidine-1,2-dicarboxylate

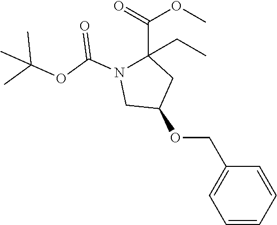 [SMILES: CCC1(C(=O)OC)C[C@@H](OCc2ccccc2)CN1C(=O)OC(C)(C)C]

To 1-(tert-butyl) 2-methyl (2R,4R)-4-(benzyloxy)pyrrolidine-1,2-dicarboxylate (2.00 g, 5.96 mmol) in THE (55 mL) was added LiHMDS (12 mL, 12 mmol) slowly at −78° C. The reaction mixture was stirred at −78° C. for 1 h, after which ethyl iodide (1.9 mL, 23.6 mmol) was added. The reaction was stirred at −78° C. for 5 min then allowed to warm to room temperature with stirring. After 3.5 h, the reaction was quenched with MeOH (55 mL). A solution of NaOH (1.91 g, 47.7 mmol) in water (55 mL) was added and the mixture was stirred at room temperature for 20 min then poured into water. The aqueous layer was extracted with EtOAc (3×). The combined organic layers were washed with brine (1×) then concentrated and purified via SiO₂ FCC (0-30% EtOAc in hexanes) to afford 1-(tert-butyl) 2-methyl (4R)-4-(benzyloxy)-2-ethylpyrrolidine-1,2-dicarboxylate (1.11 g, 51%). LCMS calcd for C₁₅H₂₂NO₃ [M-Boc+2H]⁺: m/z=264.2; Found: 264.0.

### Step 2. (4R)-4-(Benzyloxy)-1-(tert-butoxycarbonyl)-2-ethylpyrrolidine-2-carboxylic acid

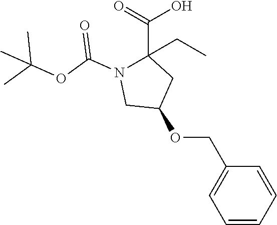 [SMILES: CCC1(C(=O)O)C[C@@H](OCc2ccccc2)CN1C(=O)OC(C)(C)C]

To 1-(tert-butyl) 2-methyl (4R)-4-(benzyloxy)-2-ethylpyrrolidine-1,2-dicarboxylate (2.72 g, 7.48 mmol) in 1:1 THF/MeOH (40 mL) was added a solution of NaOH (2.99 g, 74.8 mmol) in water (20 mL). The reaction was stirred at 75° C. for 2.5 h then allowed to cool to room temperature. The mixture was poured into water then extracted with MTBE (3×). The combined organic layers were washed with water (2×). The combined aqueous layers were extracted once more with MTBE then acidified to pH<2 with 2 N HCl (aq) and subsequently extracted with EtOAc (3×). The combined EtOAc layers were washed with brine, dried with MgSO₄, filtered then concentrated to give crude (4R)-4-(benzyloxy)-1-(tert-butoxycarbonyl)-2-ethylpyrrolidine-2-carboxylic acid (assumed quantitative yield) as a yellow oil. LCMS calcd for C₁₄H₂₀NO₃ [M-Boc+2H]⁺: m/z=250.1; Found: 250.0.

### Step 3. tert-Butyl (2R,4R)-4-(benzyloxy)-2-((4-bromo-6-chloropyridazin-3-yl)carbamoyl)-2-ethylpyrrolidine-1-carboxylate and tert-butyl (2S,4R)-4-(benzyloxy)-2-((4-bromo-6-chloro-pyridazin-3-yl)carbamoyl)-2-ethylpyrrolidine-1-carboxylate

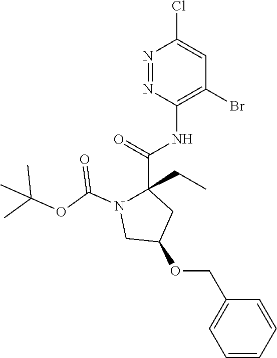 [SMILES: CC[C@@]1(C(=O)Nc2nnc(Cl)cc2Br)C[C@@H](OCc2ccccc2)CN1C(=O)OC(C)(C)C]

To crude 1-(tert-butyl) 2-methyl (4R)-4-(benzyloxy)-2-ethylpyrrolidine-1,2-dicarboxylate (672 mg, 1.43 mmol) and N,N-diisopropylethylamine (1.21 mL, 6.92 mmol) in MeCN (10 mL) was added HATU (880 mg, 2.31 mmol). The reaction was stirred at room temperature for 20 min then concentrated. The residue was taken up in THE (8 mL) then 4-bromo-6-chloropyridazin-3-amine (802 mg, 3.85 mmol) was added, followed by NaH (60% dispersion in mineral oil; 385 mg, 9.62 mmol). After rinsing the sides of the flask with THE (2 mL), the reaction was stirred at room temperature for 50 min then cooled to 0° C. and quenched with sat. NH₄Cl (aq). The mixture was poured into water then extracted with EtOAc (3×). The combined organic layers were washed with brine (1×) then concentrated. The residue was purified via SiO₂ FCC (0-100% EtOAc in hexanes) to afford “diastereomer A” tert-butyl (2R,4R)-4-(benzyloxy)-2-((4-bromo-6-chloropyridazin-3-yl)carbamoyl)-2-ethylpyrrolidine-1-carboxylate (473 mg, 46%) and “diastereomer B” tert-butyl (2S,4R)-4-(benzyloxy)-2-((4-bromo-6-chloropyridazin-3-yl)carbamoyl)-2-ethylpyrrolidine-1-carboxylate (286 mg, 28%). LCMS calcd for C₂₃H₂₉BrClN₄O₄ [M+H]⁺: m/z=539.1; Found: 539.0 (diastereomer A) and 539.0 (diastereomer B).

### Step 4. (6aR,8R)-8-(Benzyloxy)-2-chloro-6a-ethyl-6a,7,8,9-tetrahydropyrrolo[1′,2′:4,5]pyrazino[2,3-c]pyridazin-6(5H)-one and (6aS,8R)-8-(benzyloxy)-2-chloro-6a-ethyl-6a,7,8,9-tetrahydropyrrolo[1′,2′:4,5]pyrazino[2,3-c]pyridazin-6(5H)-one

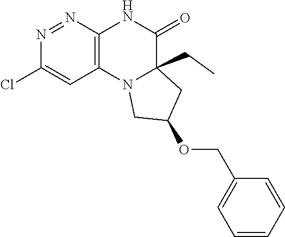 [SMILES: CC[C@@]12C[C@@H](OCc3ccccc3)CN1c1cc(Cl)nnc1NC2=O]

To tert-butyl (2R,4R)-4-(benzyloxy)-2-((4-bromo-6-chloropyridazin-3-yl)carbamoyl)-2-ethylpyrrolidine-1-carboxylate (473 mg, 0.88 mmol) in DCM (17 mL) was added TFA (17 mL, 222 mmol). The reaction was stirred at room temperature for 3 h then concentrated. The residue was dissolved in MeCN (15 mL). N,N-Diisopropylethylamine (0.76 mL, 4.38 mmol) was added and the reaction was stirred at 80° C. for 2 h then allowed to cool to room temperature overnight.

The reaction was concentrated to afford crude (6aR,8R)-8-(benzyloxy)-2-chloro-6a-ethyl-6a,7,8,9-tetrahydropyrrolo[1′,2′:4,5]pyrazino[2,3-c]pyridazin-6(5H)-one (Intermediate 11; assumed quantitative yield) which was used directly without further purification. LCMS calcd for C₁₈H₂₀ClN₄O₂[M+H]⁺: m/z=359.1; Found: 359.1.

### Step 5. (6aS,8R)-8-(Benzyloxy)-2-chloro-6a-ethyl-5,6,6a,7,8,9-hexahydropyrrolo-[1′,2′:4,5]pyrazino[2,3-c]pyridazine

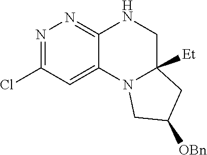 [SMILES: CC[C@]12CNc3nnc(Cl)cc3N1C[C@H](OCc1ccccc1)C2]

To a solution of (6aS,8R)-8-(benzyloxy)-2-chloro-6a-ethyl-6a,7,8,9-tetrahydropyrrolo-[1′,2′:4,5]pyrazino[2,3-c]pyridazine-6(5H)-one (1.34 g, 3.73 mmol) in THE (30 mL) was added borane dimethyl sulfide complex (3.54 mL, 37.3 mmol) at 0° C. The resulting mixture was stirred at 50° C. for 1 h and then was cooled to 0° C. MeOH (3 mL) was added slowly, and the reaction mixture was stirred at room temperature for 10 min. The mixture was concentrated and diluted with ethanol (30 mL). Sodium cyanoborohydride (1.17 g, 18.7 mmol) and acetic acid (2.14 mL, 37.3 mmol) were added, and the reaction mixture was stirred at 80° C. for 4 h. The reaction was quenched with sat. NaHCO₃ (aq.) (30 mL), and the mixture was diluted with EtOAc (30 mL). The layers were separated, and the aqueous layer was extracted with EtOAc (30 mL×3). The combined organic layers were washed with brine, dried over Na₂SO₄, filtered, and concentrated to afford the title compound (1.29 g, 3.74 mmol, 99% yield) as a yellow solid. LC-MS calc. for C₁₈H₂₂ClN₄O [M+H]⁺m/z=345.2/347.2; Found 345.4/347.5.

### Step 6. (6aS,8R)-2-Chloro-6a-ethyl-5,6,6a,7,8,9-hexahydropyrrolo[1′,2′:4,5]-pyrazino[2,3-c]pyridazin-8-ol

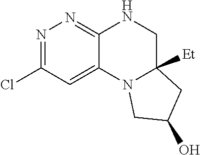 [SMILES: CC[C@]12CNc3nnc(Cl)cc3N1C[C@H](O)C2]

To a solution of (6aS,8R)-8-(benzyloxy)-2-chloro-6a-ethyl-5,6,6a,7,8,9-hexahydropyrrolo[1′,2′:4,5]pyrazino[2,3-c]pyridazine (1.45 g, 4.20 mmol) in DCM (30 mL) at −78° C. was added trichloroborane (1 N in DCM, 12.6 mL, 12.6 mmol) dropwise. The resulting mixture was slowly warmed to room temperature and stirred for 30 min. Sat. NaHCO₃ (aq.) (50 mL) was added, and the reaction mixture was diluted with DCM (20 mL). The layers were separated, and the aqueous layer was extracted with CHCl₃/IPA (3:1) (50 mL×4). The combined organic layers were dried over Na₂SO₄, filtered, and concentrated. The crude product was slurried in MTBE (100 mL) and collected by filtration to afford the title compound (1.01 g, 3.93 mmol, 93.4% yield) as a white solid. ¹H NMR (300 MHz, DMSO-d₆) δ 7.01 (d, J=4.3 Hz, 1H), 6.26 (d, J=0.9 Hz, 1H), 5.25 (d, J=3.4 Hz, 1H), 4.45 (q, J=6.0, 5.1 Hz, 1H), 3.52 (ddd, J=16.1, 11.7, 5.4 Hz, 2H), 3.28-3.11 (m, 1H), 2.61 (dd, J=11.6, 1.2 Hz, 1H), 2.00 (dd, J=13.3, 1.9 Hz, 1H), 1.86-1.68 (m, 1H), 1.68-1.57 (m, 1H), 1.55-1.41 (m, 1H), 0.82 (t, J=7.4 Hz, 3H). LC-MS calc. for C₁₁H₁₆ClN₄O [M+H]⁺m/z=255.1/257.1; Found 255.3/257.3.

### Step 7. (6aS,8S)-2-Chloro-6a-ethyl-5,6,6a,7,8,9-hexahydropyrrolo[1′,2′:4,5]pyrazin[2,3-c]pyridazin-8-yl 4-nitrobenzoate

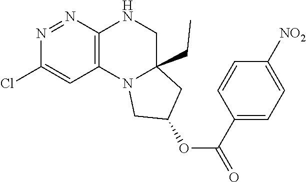 [SMILES: CC[C@]12CNc3nnc(Cl)cc3N1C[C@@H](OC(=O)c1ccc([N+](=O)[O-])cc1)C2]

To a solution of (6aS,8R)-2-chloro-6a-ethyl-5,6,6a,7,8,9-hexahydropyrrolo[1′,2′:4,5]-pyrazino[2,3-c]pyridazin-8-ol (290 mg, 1.14 mmol) in THE (6 mL) was added triphenylphosphine (597 mg, 2.28 mmol) and 4-nitrobenzoic acid (381 mg, 2.28 mmol). The resulting mixture was stirred for 5 min, and then diisopropyl azodicarboxylate (0.451 mL, 2.28 mmol) was added dropwise. The resulting mixture was stirred for 1 h. The reaction mixture was concentrated, and the crude material was purified by silica gel chromatography (0-70% EtOAc/heptanes) to afford the title compound (430 mg, 1.06 mmol, 93.5% yield) as a white solid. LC-MS calc. for C₁₈H₁₉ClN₅O₄[M+H]⁺m/z=404.1/406.1; Found 404.4/406.4.

### Step 8. (6aS,8S)-2-Chloro-6a-ethyl-5,6,6a,7,8,9-hexahydropyrrolo[1′,2′:4,5]pyrazino-[2,3-c]pyridazin-8-ol

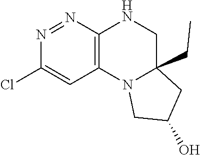 [SMILES: CC[C@]12CNc3nnc(Cl)cc3N1C[C@@H](O)C2]

To a solution of (6aS,8S)-2-chloro-6a-ethyl-5,6,6a,7,8,9-hexahydropyrrolo[1′,2′:4,5]-pyrazino[2,3-c]pyridazin-8-yl 4-nitrobenzoate (430 mg, 1.06 mmol) in methanol (4 mL) and THE (4 mL) was added potassium carbonate (442 mg, 3.19 mmol). The resulting mixture was stirred overnight. The reaction mixture was concentrated, and 2 N HCl (aq.) (20 mL) and EtOAc (20 mL) were added. The layers were separated, and the aqueous layer was extracted with EtOAc (20 mL). The combined organic layers were discarded, and the aqueous layer was adjusted to pH 7-8 with solid sodium bicarbonate. The resulting solution was extracted with EtOAc (20 mL×3). The combined organic layers were washed with brine, dried over Na₂SO₄, filtered, and concentrated to afford the title compound (220 mg, 0.864 mmol, 81.1% yield) as a white solid. LC-MS calc. for C₁₁H₁₆ClN₄O [M+H]⁺m/z=255.1/257.1; Found 255.5/257.3.

### Step 9. 6-Hydroxy-5-methylnicotinic acid

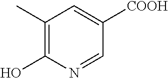

To a solution of 6-fluoro-5-methylnicotinic acid (1.0 g, 6.5 mmol) in water (20 mL) was added sodium hydroxide (1.29 g, 32.2 mmol). The resulting mixture was stirred at 90° C. overnight. The reaction mixture was cooled to room temperature, and the mixture was adjusted to pH 3-4 with 1 N HCl (aq.), producing a white precipitate. The solid was collected by filtration and co-evaporated with toluene (2×) to afford the title compound (930 mg, 6.0 mmol, 93% yield) as a white solid. LC-MS calc. for C₇H₈NO₃ [M+H]⁺m/z=154.0; Found 154.1.

### Step 10. Methyl 6-hydroxy-5-methylnicotinate

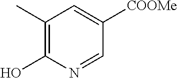

To a solution of 6-hydroxy-5-methylnicotinic acid (930 mg, 6.0 mmol) in methanol (20 mL) was added sulfuric acid (0.32 mL, 6.0 mmol). The reaction mixture was stirred at reflux overnight. The mixture was cooled to room temperature, and the resulting solid was collected by filtration, washed with MeOH, and concentrated to afford the title compound (610 mg, 3.7 mmol, 61% yield) as a white solid. LC-MS calc. for C₈H₁₀NO₃ [M+H]⁺m/z=168.1; Found 168.2.

### Step 11. Methyl 6-(((6aS,8R)-2-chloro-6a-ethyl-5,6,6a,7,8,9-hexahydropyrrolo[1′,2′:4,5]-pyrazino[2,3-c]pyridazin-8-yl)oxy)-5-methylnicotinate

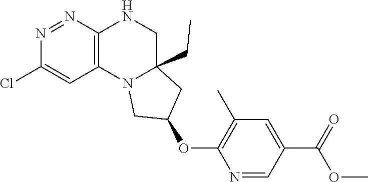 [SMILES: CC[C@]12CNc3nnc(Cl)cc3N1C[C@H](Oc1ncc(C(=O)OC)cc1C)C2]

To a solution of (6aS,8S)-2-chloro-6a-ethyl-5,6,6a,7,8,9-hexahydropyrrolo[1′,2′:4,5]-pyrazino[2,3-c]pyridazin-8-ol (45.5 mg, 0.179 mmol) in THF (2 mL) was added triphenylphosphine (93.7 mg, 0.357 mmol) and methyl 6-hydroxy-5-methylnicotinate (59.7 mg, 0.357 mmol). The reaction mixture was stirred for 10 min at room temperature. Diisopropyl azodicarboxylate (0.0703 mL, 0.357 mmol) was added at 0° C., and the reaction mixture was stirred at room temperature for 3 h. The reaction mixture was concentrated, and the crude material was purified by silica gel chromatography (20-100% EtOAc/heptanes) to afford the title compound (70 mg, 0.17 mmol, 97% yield) as a colorless oil. LC-MS calc. for C₁₉H₂₃ClN₅O₃ [M+H]⁺m/z=404.1/406.1; Found 404.2/406.4.

### Step 12: (6-(((6aS,8R)-2-Chloro-6a-ethyl-5,6,6a,7,8,9-hexahydropyrrolo[1′,2′:4,5]-pyrazino[2,3-c]pyridazin-8-yl)oxy)-5-methylpyridin-3-yl)methanol

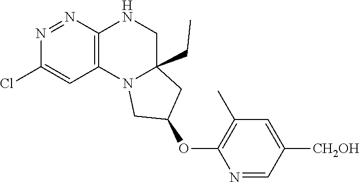 [SMILES: CC[C@]12CNc3nnc(Cl)cc3N1C[C@H](Oc1ncc(CO)cc1C)C2]

To a solution of methyl 6-(((6aS,8R)-2-chloro-6a-ethyl-5,6,6a,7,8,9-hexahydropyrrolo-[1′,2′:4,5]pyrazino[2,3-c]pyridazin-8-yl)oxy)-5-methylnicotinate (70.0 mg, 0.173 mmol) in THE at −78° C. (3 mL) was added lithium diisobutyl-tert-butoxyaluminum hydride (2.5 N in THF/hexanes, 3.47 mL, 0.867 mmol) slowly. The resulting mixture was stirred at −78° C. for 10 min, then slowly warmed to room temperature, and stirred for 30 min. The reaction was quenched with MeOH (2 mL), and the reaction mixture was stirred for 30 min. The reaction mixture was diluted with water (20 mL) and EtOAc (20 mL). The layers were separated, and the aqueous layer was extracted with EtOAc (20 mL×3). The combined organic layers were washed with brine, dried over Na₂SO₄, filtered, and concentrated to afford the title compound (61.0 mg, 0.162 mmol, 93.6% yield) as a white solid. LC-MS calc. for C₁₈H₂₃ClN₅O₂[M+H]⁺m/z=376.2/378.2; Found 376.4/378.5.

### Step 13: 6-(((6aS,8R)-2-Chloro-6a-ethyl-5,6,6a,7,8,9-hexahydropyrrolo[1′,2′:4,5]-pyrazino[2,3-c]pyridazin-8-yl)oxy)-5-methylnicotinaldehyde

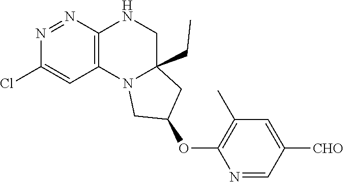 [SMILES: CC[C@]12CNc3nnc(Cl)cc3N1C[C@H](Oc1ncc(C=O)cc1C)C2]

To a solution of (6-(((6aS,8R)-2-chloro-6a-ethyl-5,6,6a,7,8,9-hexahydropyrrolo[1′,2′:4,5]-pyrazino[2,3-c]pyridazin-8-yl)oxy)-5-methylpyridin-3-yl)methanol (61.0 mg, 0.160 mmol) in CHCl₃ (5 mL) was added manganese(IV) oxide (282 mg, 3.25 mmol). The reaction mixture was stirred at 50° C. for 2 h. The mixture was cooled to room temperature, filtered through Celite, and concentrated. The crude material was further purified by silica gel chromatography (20-100% EtOAc/heptanes) to afford the title compound (37.0 mg, 0.0990 mmol, 61.0% yield) as a white solid. LC-MS calc. for C₁₈H₂₁ClN₅O₂ [M+H]⁺m/z=374.1/376.1; Found 374.2/376.2.

### Step 14: 6-(((6aS,8R)-6a-Ethyl-2-(2-hydroxyphenyl)-5,6,6a,7,8,9-hexahydropyrrolo-[1′,2′:4,5]pyrazino[2,3-c]pyridazin-8-yl)oxy)-5-methylnicotinaldehyde

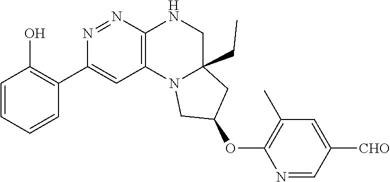 [SMILES: CC[C@]12CNc3nnc(-c4ccccc4O)cc3N1C[C@H](Oc1ncc(C=O)cc1C)C2]

To a solution of 6-(((6aS,8R)-2-chloro-6a-ethyl-5,6,6a,7,8,9-hexahydropyrrolo[1′,2′:4,5]-pyrazino[2,3-c]pyridazin-8-yl)oxy)-5-methylnicotinaldehyde (37.0 mg, 0.0990 mmol) in 1,4-dioxane (2 mL) and water (0.10 mL) was added 2-hydroxyphenylboronic acid (41.0 mg, 0.297 mmol), XPhos-Pd-G2 (7.8 mg, 0.0099 mmol, CAS 1310584-14-5), and cesium acetate (76.0 mg, 0.396 mmol). The reaction mixture was sparged with N₂ for 10 min and stirred at 95° C. for 2 h. The reaction was cooled to room temperature and concentrated. The crude material was diluted with DMSO (3 mL) and centrifuged. The liquid was collected and directly purified by prep-HPLC (10.0-60.0% MeCN/0.1% TFA (aq)) to afford the title compound as a TFA salt (32.0 mg, 0.0742 mmol, 74.9% yield), a brown solid. LC-MS calc. for C₂₄H₂₆N₅O₃ [M+H]⁺m/z=432.2; Found 432.3.

### Step 15: (S)-3-(6-(4-((6-(((6aS,8R)-6a-Ethyl-2-(2-hydroxyphenyl)-5,6,6a,7,8,9-hexahydropyrrolo[1′,2′:4,5]pyrazino[2,3-c]pyridazin-8-yl)oxy)-5-methylpyridin-3-yl)methyl)-piperazin-1-yl)-1-oxoisoindolin-2-yl)piperidine-2,6-dione

To a solution of (S)-3-(1-oxo-6-(piperazin-1-yl)isoindolin-2-yl)piperidine-2,6-dione (24.4 mg, 0.0742 mmol) in DMSO (2 mL) was added 6-(((6aS,8R)-6a-ethyl-2-(2-hydroxyphenyl)-5,6,6a,7,8,9-hexahydropyrrolo[1′,2′:4,5]pyrazino[2,3-c]pyridazin-8-yl)oxy)-5-methylnicotinaldehyde (16.0 mg, 0.0371 mmol) and N,N-diisopropylethylamine (0.0646 mL, 0.371 mmol). After stirring for 20 min, sodium triacetoxyborohydride (31.4 mg, 0.148 mmol) was added. The reaction mixture was stirred at 40° C. overnight and then directly purified by prep-HPLC (10-60% MeCN/0.1% TFA (aq)) to afford the title compound as a TFA salt (8.7 mg, 0.012 mmol, 32% yield), a white solid. ¹H NMR (300 MHz, DMSO-d₆) δ 10.99 (s, 1H), 8.24 (s, 1H), 8.07-7.86 (m, 2H), 7.59 (d, J 2.2 Hz, 1H), 7.45 (d, J 8.4 Hz, 1H), 7.37-7.13 (m, 4H), 6.98 (s, 1H), 6.85-6.81 (m, 2H), 5.80 (t, J 6.3 Hz, 1H), 5.11 (dd, J 13.3, 5.1 Hz, 1H), 4.35 (d, J 16.8 Hz, 1H), 4.22 (d, J 16.8 Hz, 1H), 4.03 (dd, J 13.1, 6.2 Hz, 1H), 3.73-3.49 (m, 8H), 3.23-3.20 (m, 3H), 2.97-2.81 (m, 2H), 2.65-2.56 (m, 2H), 2.45-2.30 (m, 2H), 2.19 (s, 3H), 2.08-1.94 (m, 2H), 1.87 (dd, J=13.8, 7.3 Hz, 1H), 1.68 (dd, J=13.8, 7.2 Hz, 1H), 1.49-1.44 (m, 1H), 0.91 (q, J 7.0 Hz, 3H). LC-MS calc. for C₄₁H₄₆N₉O₅ [M+H]⁺m/z=744.4; Found 746.6.

### 1B. (2S,4R)-4-Hydroxy-1-((R)-2-(3-(2-(4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

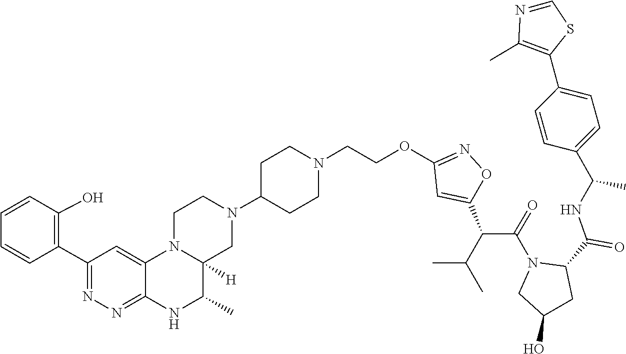 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@@H](C)[C@@H]5C4)CC3)no2)C(C)C)cc1]

### Step 1: (2S,4R)-4-((tert-butyldimethylsilyl)oxy)-1-((R)-2-(3-(2-(4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

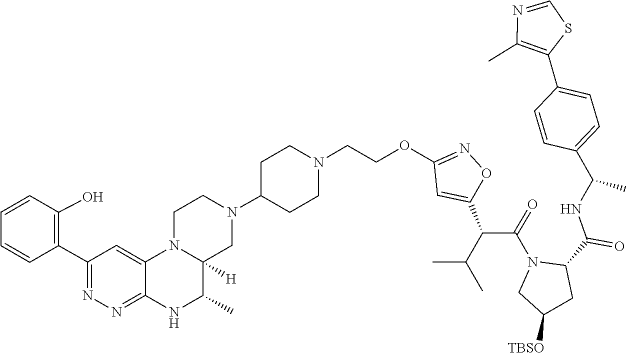 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCN3CCC(N4CCN5c6cc(-c7ccccc7O)nnc6N[C@@H](C)[C@@H]5C4)CC3)no2)C(C)C)cc1]

To a solution of 2-((6S,6aS)-6-methyl-8-(piperidin-4-yl)-6,6a,7,8,9,10-hexahydro-5H-pyrazino[1′,2′:4,5]pyrazino[2,3-c]pyridazin-2-yl)phenol TFA salt (Intermediate 8; 30.0 mg, 0.0493 mmol) in DMF (2.0 mL) and water (0.10 mL) was added NaHCO₃ (25.5 mg, 0.197 mmol), followed by (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide (Intermediate 2; 51.6 mg, 0.0788 mmol) and sodium triacetoxyborohydride (33.4 mg, 0.158 mmol). The mixture was stirred at rt for 12 h then purified by prep-HPLC on a C18 column (5-50% MeCN/0.05% formic acid (aq.)) to afford the formate salt of the title compound (50.0 mg, 95.2% yield). LCMS calc. for C₅₄H₇₅N₁₀O₆SiS [M+H]⁺m/z=1019.5; Found 1019.5.

### Step 2: (2S,4R)-4-Hydroxy-1-((R)-2-(3-(2-(4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

To a solution of (2S,4R)-4-((tert-butyldimethylsilyl)oxy)-1-((R)-2-(3-(2-(4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide formate salt (50.0 mg, 0.0469 mmol) in THE (1.0 mL) and IPA (1.0 mL) was added conc. HCl (aq.) (0.50 mL). The mixture was stirred at rt for 15 min then the crude mixture was purified by prep-HPLC on a C18 column (5-50% MeCN/0.05% formic acid (aq.)) to afford the formate salt of the title compound (32.9 mg, 73.8% yield). LCMS calc. for C₄₈H₆₁N₁₀O₆S [M+H]⁺m/z=905.4; Found 905.4.

The HCl salt of the title compound can be prepared by treating the combined fractions containing the formate salt of title compound from the prep-HPLC purification with HCl, followed by lyophilization.

The free-base of title compound was obtained by the following procedure: To a solution of the HCl salt of the title compound in DCM and water (1:1) was added NaHCO₃ until a pH of 7 was obtained. The organic layer was separated, and the aqueous layer was extracted with DCM (2×). The combined organic layers were dried with Na₂SO₄ then concentrated to afford the title compound as its free-base. ¹H NMR (400 MHz, CDCl₃) δ 8.67 (s, 1H), 7.56 (d, J=7.6 Hz, 1H), 7.45 (d, J=7.9 Hz, 1H), 7.40 (d, J=8.9 Hz, 2H), 7.36 (d, J=8.3 Hz, 2H), 7.24 (m, 1H), 7.01 (d, J=8.3 Hz, 1H), 6.90-6.84 (m, 2H), 5.94 (s, 1H), 5.69 (br s, 1H), 5.51 (m, 1H), 4.70-4.58 (m, 2H), 4.42 (m, 2H), 3.77 (dd, J=10.8, 5.3 Hz, 1H), 3.70 (m, 2H), 3.61 (dd, J=10.7, 2.8 Hz, 1H), 3.50 (m, 1H), 3.46 (m, 1H), 3.18 (m, 4H), 3.10-2.86 (m, 5H), 2.53 (s, 3H), 2.55-2.37 (m, 4H), 2.34-2.15 (m, 2H), 2.13-1.94 (m, 2H), 1.92-1.66 (m, 4H), 1.49 (d, J=7.4 Hz, 3H), 1.31 (d, J=6.0 Hz, 3H), 1.04 (d, J=6.4 Hz, 3H), 0.92 (d, J=6.4 Hz, 3H).

### 1C. (2S,4R)-4-Hydroxy-1-((R)-2-(3-(2-(4-((6R,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

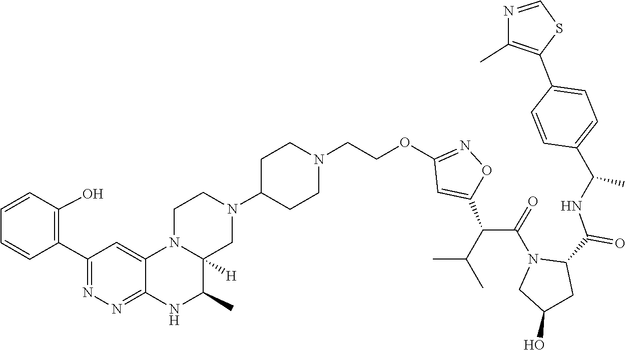 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@H](C)[C@@H]5C4)CC3)no2)C(C)C)cc1]

To a solution of 2-((6R,6aS)-6-methyl-8-(piperidin-4-yl)-6,6a,7,8,9,10-hexahydro-5H-pyrazino[1′,2′:4,5]pyrazino[2,3-c]pyridazin-2-yl)phenol TFA salt (Intermediate 7; 15.7 mg, 0.0188 mmol) and (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide (Intermediate 2; 12.3 mg, 0.0188 mmol) in DMF (1.0 mL) and water (50 μL) was added NaHCO₃ (6.31 mg, 0.0751 mmol) and sodium triacetoxyborohydride (7.95 mg, 0.0375 mmol). The mixture was stirred at rt for 1 h then purified via prep-HPLC on a C18 column (10-40% MeCN/0.05% TFA (aq.)). The combined fractions containing the desired product were concentrated to −10 mL then treated with 0.1 mL TFA and lyophilized to give a crude residue which was purified via prep-HPLC on a C₁₈ column (10-40% MeCN/0.05% TFA (aq.)) to give the TFA salt of the title compound (6.50 mg, 34.0%) as a colorless solid. LC-MS calc. for C₄₈H₆₁N₁₀O₆S [M+H]⁺: m/z=905.4; Found 905.4.

### 1D. (2S,4R)-4-Hydroxy-1-((R)-2-(3-(2-(4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

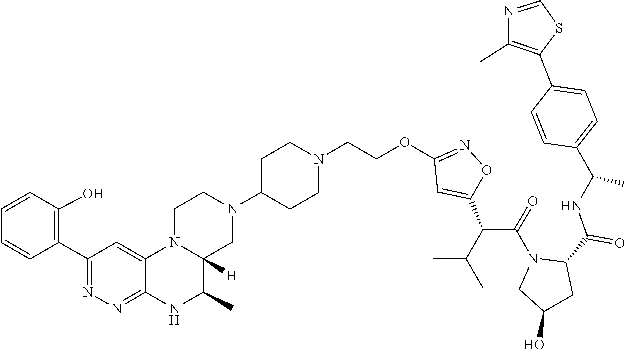 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@H](C)[C@H]5C4)CC3)no2)C(C)C)cc1]

### Step 1: (2S,4R)-4-((tert-Butyldimethylsilyl)oxy)-1-((R)-2-(3-(2-(4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

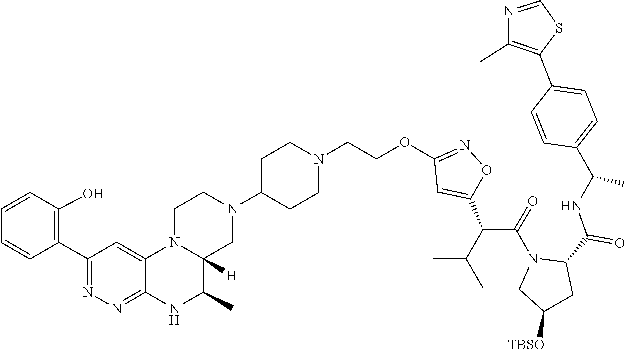 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCN3CCC(N4CCN5c6cc(-c7ccccc7O)nnc6N[C@H](C)[C@H]5C4)CC3)no2)C(C)C)cc1]

A mixture of tert-butyl 4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidine-1-carboxylate HCl salt (Intermediate 10; 16.0 mg, 0.0353 mmol), (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide (Intermediate 2; 21.0 mg, 0.0321 mmol), and NaHCO₃ (6.73 mg, 0.0802 mmol) in DMF (180 μL) and water (10 μL) was stirred at rt for 1 h then treated with sodium triacetoxyborohydride (13.6 mg, 0.0641 mmol), portion-wise, over 10 min. The reaction was stirred for 2 h then diluted with DMSO and water and purified via prep-HPLC on a C18 column (15-30% MeCN/0.02% formic acid (aq.)). The fractions containing the desired product were combined and basified to pH 8 with 5% NaHCO₃ (aq.) and the acetonitrile was removed under reduced pressure. The resulting aqueous layer was extracted with EtOAc (3×) and the combined organic layers were dried with Na₂SO₄ then concentrated to afford the free-base of the title compound (12.0 mg, 36.7%). LCMS calc. for C₅₄H₇₅N₁₀O₆SSi [M+H]⁺m/z=1019.5; Found 1019.5.

### Step 2: (2S,4R)-4-Hydroxy-1-((R)-2-(3-(2-(4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

To a solution of (2S,4R)-4-((tert-butyldimethylsilyl)oxy)-1-((R)-2-(3-(2-(4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (6.00 mg, 0.0589 mmol) in MeOH (50 μL) was added HCl (4.0 M in dioxane; 15 μL, 58.9 μmol). The mixture was stirred at rt for 30 min then was concentrated, taken up in DMSO, and purified via prep-HPLC on a C18 column (15-30% MeCN/0.02% formic acid (aq.)). Fractions containing the desired product were combined and concentrated, then treated with 0.1% TFA (aq.) and lyophilized to give the TFA salt of the title compound (2.90 mg, 53.8%). LCMS calc. for C₄₈H₆₁N₁₀O₆S [M+H]⁺m/z=905.4; Found 905.5.

### 1E. (2S,4R)-4-Hydroxy-1-((R)-2-(3-(2-(4-((6S,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

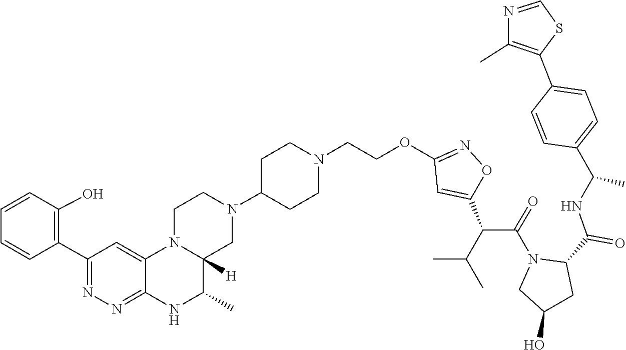 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@@H](C)[C@H]5C4)CC3)no2)C(C)C)cc1]

The title compound was synthesized by procedures analogous to those outlined in Compound I, Steps 1-2, utilizing 2-((6S,6aR)-6-methyl-8-(piperidin-4-yl)-6,6a,7,8,9,10-hexahydro-5H-pyrazino[1′,2′:4,5]pyrazino[2,3-c]pyridazin-2-yl)phenol (Intermediate 9) instead of 2-((6S,6aS)-6-methyl-8-(piperidin-4-yl)-6,6a,7,8,9,10-hexahydro-5H-pyrazino[1′,2′:4,5]pyrazino[2,3-c]pyridazin-2-yl)phenol (Intermediate 8). LCMS calc. for C₄₈H₆₁N₁₀O₆S [M+H]⁺m/z=905.4; Found 905.4.

### Example 2. Synthesis of Intermediates

### Intermediate 1. (6aS,8R)-2-Chloro-6a-methyl-5,6,6a,7,8,9-hexahydropyrrolo[1′,2′:4,5]pyrazino[2,3-c]pyridazin-8-ol

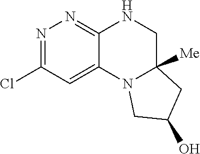 [SMILES: C[C@]12CNc3nnc(Cl)cc3N1C[C@H](O)C2]

### Step 1: 2-Benzyl 1-(tert-butyl) (2S,4R)-4-hydroxypyrrolidine-1,2-dicarboxylate

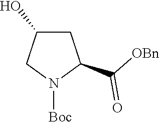 [SMILES: CC(C)(C)OC(=O)N1C[C@H](O)C[C@H]1C(=O)OCc1ccccc1]

To a mixture of (2S,4R)-1-(tert-butoxycarbonyl)-4-hydroxypyrrolidine-2-carboxylic acid (25.0 g, 108 mmol) and K₂CO₃ (44.8 g, 324 mmol) in DMF (250 mL) was added benzyl bromide (26.7 mL, 216 mmol). The reaction was stirred at room temperature for 2 h then diluted with MTBE (500 mL) and filtered through a pad of Celite®. The filtrate was dried over Na₂SO₄, filtered then concentrated to give crude give 2-benzyl 1-(tert-butyl) (2S,4R)-4-hydroxypyrrolidine-1,2-dicarboxylate (assumed quantitative yield) which was used without further purification. LCMS calcd for C₁₇H₂₄NO₅ [M+H]⁺: m/z=322.2; Found: 322.5.

### Step 2: 2-Benzyl 1-(tert-butyl) (2S,4R)-4-(benzyloxy)pyrrolidine-1,2-dicarboxylate

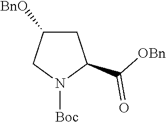 [SMILES: CC(C)(C)OC(=O)N1C[C@H](OCc2ccccc2)C[C@H]1C(=O)OCc1ccccc1]

To crude 2-benzyl 1-(tert-butyl) (2S,4R)-4-hydroxypyrrolidine-1,2-dicarboxylate (34.7 g, 108 mmol) in DMF (300 mL) was added benzyl bromide (19.2 mL, 162 mmol) then KOH (18.2 g, 324 mmol). The reaction was stirred at room temperature for 3 h then diluted with MTBE (500 mL) and filtered through a pad of Celite®. The filtrate was washed with 1 N HCl (aq; 500 mL), sat. NaHCO₃ (aq; 500 mL), then brine (500 mL). The organic layer was dried over Na₂SO₄, filtered and concentrated. The residue was purified via SiO₂ FCC (0-40% MTBE in heptanes) to give 2-benzyl 1-(tert-butyl) (2S,4R)-4-(benzyloxy)pyrrolidine-1,2-dicarboxylate (26.0 g, 59%) as a colorless oil. LCMS calcd for C₂₄H₃₀NO₅ [M+H]⁺: m/z=412.2; Found: 412.5.

### Step 3: 2-Benzyl 1-(tert-butyl) (4R)-4-(benzyloxy)-2-methylpyrrolidine-1,2-dicarboxylate

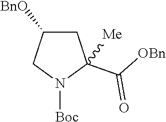 [SMILES: CC(C)(C)OC(=O)N1C[C@H](OCc2ccccc2)CC1(C)C(=O)OCc1ccccc1]

To 2-benzyl 1-(tert-butyl) (2S,4R)-4-(benzyloxy)pyrrolidine-1,2-dicarboxylate (34.7 g, 84.3 mmol) in THE (8 mL) at 0° C. was added methyl iodide (6.3 mL, 101 mmol) portion-wise. The reaction was stirred for 30 min, after which LiHMDS (16.9 g, 101 mmol) was added. The reaction was allowed to warm to room temperature, stirred for 12 h, then quenched with sat. NH₄Cl (aq; 200 mL) and extracted with MTBE (3×200 mL). The combined organic phase was dried over Na2SO₄, filtered and concentrated. The residue was purified via SiO₂ FCC (0-20% MTBE in heptanes) to give 2-benzyl 1-(tert-butyl) (4R)-4-(benzyloxy)-2-methylpyrrolidine-1,2-dicarboxylate (33.2 g, 93%) as a colorless, viscous oil. LCMS calcd for C₂₅H₃₂NO₅ [M+H]⁺: m/z=426.2; Found: 426.1.

### Step 4: (4R)-4-(Benzyloxy)-1-(tert-butoxycarbonyl)-2-methylpyrrolidine-2-carboxylic acid

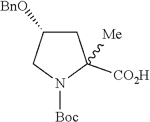 [SMILES: CC(C)(C)OC(=O)N1C[C@H](OCc2ccccc2)CC1(C)C(=O)O]

To 2-benzyl 1-(tert-butyl) (4R)-4-(benzyloxy)-2-methylpyrrolidine-1,2-dicarboxylate (33.2 g, 78.0 mmol) in MeOH (150 mL) at 0° C. was added NaOH (12.5 g, 312 mmol) in water (150 mL) dropwise. The reaction was stirred at 75° C. for 12 h, after which MeOH was removed via rotary evaporation. The resultant aqueous phase was diluted with water (150 mL), washed with 2:1 heptanes/MTBE (3×150 mL), then acidified to pH -2 with 4 N HCl (aq) and extracted with MTBE (3×150 mL). The combined organic phase from the latter three MTBE extractions was dried over Na2SO₄, filtered and concentrated to afford (4R)-4-(benzyloxy)-1-(tert-butoxycarbonyl)-2-methylpyrrolidine-2-carboxylic acid (27.0 g, quant) which was used without further purification. LCMS calcd for C₁₈H₂₆NO₅ [M+H]⁺: m/z=336.2; Found: 336.3.

### Step 5: (6R,7aS)-6-(benzyloxy)-7a-methyltetrahydro-1H,3H-pyrrolo[1,2-c]oxazole-1,3-dione

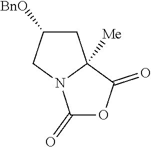 [SMILES: C[C@@]12C[C@@H](OCc3ccccc3)CN1C(=O)OC2=O]

To a solution of DMF (4.5 mL, 58 mmol) in DCM (20 mL) at 0° C. was added oxalyl chloride (4.32 mL, 50.4 mmol) dropwise. The reaction was stirred at 0° C. for 30 min, after which a solution of (4R)-4-(benzyloxy)-1-(tert-butoxycarbonyl)-2-methylpyrrolidine-2-carboxylic acid (13.0 g, 38.8 mmol) and pyridine (3.12 mL, 38.8 mmol) in DCM (100 mL) was added, dropwise. After an additional 30 min of stirring at 0° C., the reaction was washed with cold water (100 mL), then dried over Na₂SO₄, filtered and concentrated. The residue was purified via SiO2 FCC (0-100% EtOAc in heptanes) to give “diastereomer A” and the desired “diastereomer B” (6R,7aS)-6-(benzyloxy)-7a-methyltetrahydro-1H,3H-pyrrolo[1,2-c]oxazole-1,3-dione (820 mg, 8%) as colorless, viscous oils.

### Step 6:(2S,4R)-4-(Benzyloxy)-N-(4-bromo-6-chloropyridazin-3-yl)-2-methylpyrrolidine-2-carboxamide

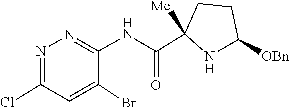 [SMILES: C[C@@]1(C(=O)Nc2nnc(Cl)cc2Br)CC[C@@H](OCc2ccccc2)N1]

To a cold solution of (6R,7aS)-6-(benzyloxy)-7a-methyltetrahydro-1H,3H-pyrrolo[1,2-c]oxazole-1,3-dione (diastereomer B) (820 mg, 3.14 mmol) and 4-bromo-6-chloropyridazin-3-amine (0.59 g, 2.8 mmol) in THF (4 mL), NaH (60% suspension in mineral oil; 0.188 g, 4.71 mmol) was added in portions at 0° C. When HPLC analysis indicated the reaction had stalled, additional NaH (60% suspension in mineral oil; 0.093 g, 2.4 mmol) was added. HPLC analysis indicated improved conversion. The reaction was quenched with a 10% citric acid (aq.) (20 mL), and the mixture extracted with 3:1 CHCl₃/IPA (15 mL×5). The combined organic phase was dried over Na₂SO₄, filtered, and concentrated to provide the crude (2S,4R)-4-(benzyloxy)-N-(4-bromo-6-chloropyridazin-3-yl)-2-methylpyrrolidine-2-carboxamide (assumed quantitative yield) which was used for next reaction without further purification. LCMS calculated for C₁₇H₁₉BrClN₄O₂[M+H]⁺: m/z=425.0; found: 425.2.

### Step 7: (6aS,8R)-8-(Benzyloxy)-2-chloro-6a-methyl-6a,7,8,9-tetrahydropyrrolo[1′,2′:4,5]pyrazino[2,3-c]pyridazin-6(5H)-one

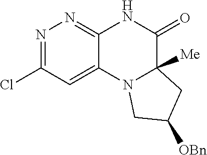 [SMILES: C[C@@]12C[C@@H](OCc3ccccc3)CN1c1cc(Cl)nnc1NC2=O]

To crude (2S,4R)-4-(benzyloxy)-N-(4-bromo-6-chloropyridazin-3-yl)-2-methylpyrrolidine-2-carboxamide in THE (20 mL) was added N-methylmorpholine (228 mg, 2.26 mmol) slowly. The reaction mixture was stirred overnight at 120° C. then concentrated. The residue was taken up in water (10 mL) and DCM (10 mL), and the organic layer was separated. The aqueous layer was extracted with DCM (3×50 mL), and the combined organic phase was dried over Na₂SO₄, filtered, and concentrated. The residue was purified via SiO₂ FCC (30-50% EtOAc in heptanes) to afford (6aS,8R)-8-(benzyloxy)-2-chloro-6a-methyl-6a,7,8,9-tetrahydropyrrolo[1′,2′:4,5]pyrazino[2,3-c]pyridazin-6(5H)-one (255 mg, 23.8% for two steps), as a white solid. LCMS calculated for C₁₇H₁₈ClN₄O₂[M+H]⁺: m/z=345.1; found: 345.4.

### Step 8: (6aS,8R)-8-(Benzyloxy)-2-chloro-6a-methyl-5,6,6a,7,8,9-hexahydropyrrolo [1′,2′:4,5]pyrazino[2,3-c]pyridazine

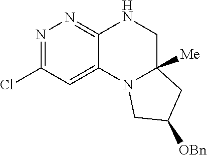 [SMILES: C[C@]12CNc3nnc(Cl)cc3N1C[C@H](OCc1ccccc1)C2]

To (6aS,8R)-8-(benzyloxy)-2-chloro-6a-methyl-6a,7,8,9-tetrahydropyrrolo [1′,2′:4,5]pyrazino[2,3-c]pyridazine-6(5H)-one (260 mg, 0.74 mmol) in THF (3 mL) was added BH₃ ·SMe₂ (0.35 mL, 3.7 mmol) at 0° C. The reaction mixture was warmed to 50° C. and stirred for 90 min. The LCMS analysis indicated full consumption of starting material. The reaction mixture was cooled to 0° C. and quenched slowly with MeOH and concentrated. The crude intermediate was then dissolved in EtOH (3 mL). NaBH₃CN (279 mg, 4.44 mmol) and AcOH (0.51 mL, 8.9 mmol) were added at 0° C. The reaction mixture was slowly heated to 80° C. and refluxed overnight. The LCMS analysis indicated consumption of the starting material and the formation of the desired product. The reaction mixture was allowed to cool to room temperature and poured into cold sat. NaHCO₃ (aq.) (25 mL). The organic layer was separated, and the aqueous layer extracted with DCM (20 mL×3). The combined organic phase was dried over Na₂SO₄, filtered, and concentrated. The residue was purified via SiO₂ FCC (10˜60% EtOAc in DCM) to give (6aS,8R)-8-(benzyloxy)-2-chloro-6a-methyl-5,6,6a,7,8,9-hexahydropyrrolo[1′,2′:4,5]pyrazino[2,3-c]pyridazine (210 mg, 0.635 mmol, 85.8% yield) as colorless viscous oil. LCMS calculated for C₁₇H₂₀ClN₄O [M+H]⁺: m/z=331.1; found: 331.5.

### Step 9: (6aS,8R)-2-Chloro-6a-methyl-5,6,6a,7,8,9-hexahydropyrrolo[1′,2′:4,5]pyrazino[2,3-c]pyridazine-8-ol

Trichloroborane (1M in DCM, 425 mg, 3.63 mmol) was added dropwise to a solution of (6aS,8R)-8-(benzyloxy)-2-chloro-6a-methyl-5, 6,6a,7,8,9-hexahydropyrrolo[1′,2′:4,5]pyrazino[2,3-c]pyridazine (240 mg, 0.73 mmol) in DCM (3 mL) at −78° C. . The resulting mixture was stirred at −78° C. for 30 min, and the reaction quenched with MeOH (10 mL). The volatiles were evaporated, the residue was basified by addition of 10% Na₂CO₃ (aq.), and the mixture was then extracted with CHCl₃/IPA (3:1, 10 mL×6). The combined organic phase was dried over Na₂SO₄, filtered, and concentrated. The residue was triturated in MTBE (5 mL) for 30 min. The solid material was filtered, washed with MTBE, and dried under high vacuum to give the title compound (150 mg, 0.62 mmol, 86% yield) as a white solid. LCMS calculated for C₁₀H₁₄ClN₄O [M+H]⁺: m/z=241.1; found: 241.3.

### Intermediate 2. (2S,4R)-4-((tert-Butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide

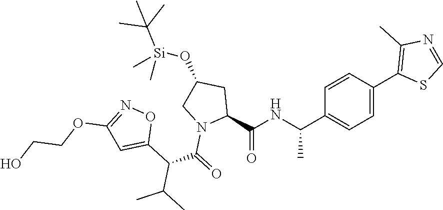 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCO)no2)C(C)C)cc1]

### Step 1: 2-(Trityloxy)ethan-1-ol

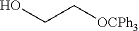

To a round bottom flask was added DCM (700 mL), triethylamine (101 mL, 727 mmol), and ethylene glycol (201 mL, 3600 mmol). The mixture was cooled to 0° C., and a solution of triphenylchloromethane (100 g, 359 mol) in DCM (300 mL) was added dropwise to maintain the temperature <5° C. After the addition was complete, the mixture was warmed to room temperature and stirred overnight. Water (1.5 L) was added to the reaction solution, and the layers were separated. The organic phase was washed with water (3×1 L). The combined organic layers were dried over anhydrous Na₂SO₄ and concentrated. The crude material was purified by silica column chromatography (20% DCM/hexanes) to afford the title compound (73.3 g, 241 mol, 67.1% yield) as a colorless oil.

### Step 2: Methyl 2-(3-(2-(trityloxy)ethoxy)isoxazol-5-yl)acetate

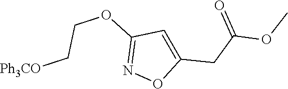

To a round bottom flask was added methyl 2-(3-oxo-2,3-dihydroisoxazol-5-yl)acetate (100 g, 637 mmol), 2-(trityloxy)ethan-1-ol (213 g, 700 mmol) and anhydrous THE (1000 mL). The reaction mixture was purged with nitrogen (3×), and then triphenylphosphine (182 g, 694 mmol) was added. The resulting mixture was cooled to −40° C., and diisopropyl azodicarboxylate (75.8 mL, 692 mmol) was added dropwise. After the addition, the reaction mixture was stirred overnight at room temperature. The reaction was quenched with sat. NaHCO₃ (aq.) (2 L), and the layers were separated. The aqueous layer was extracted with EtOAc (2×1 L). The combined organic layers were washed with brine (1 L), dried over MgSO₄, and concentrated. The crude product was purified by silica gel column chromatography (10-25% EtOAc/hexanes) to afford the title compound (250 g, 560 mmol, 89% yield). ¹H-NMR (400 MHz, CDCl₃) δ 7.54-7.41 (m, 6H), 7.37-7.19 (m, 9H), 5.97 (d, J=0.5 Hz, 1H), 4.46-4.35 (m, 2H), 3.76 (s, 3H), 3.73 (s, 2H), 3.47-3.39 (m, 2H).

### Step 3: Methyl 3-methyl-2-(3-(2-(trityloxy)ethoxy)isoxazol-5-yl)butanoate

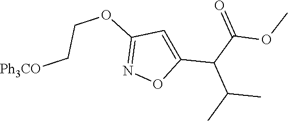

To a round bottom flask was added methyl 2-(3-(2-(trityloxy)ethoxy)isoxazol-5-yl)acetate (300 g, 676 mmol) and anhydrous THF (1.5 L). The reaction mixture was purged with nitrogen (3×). The mixture was cooled to −10° C., and potassium tert-butoxide (91.2 g, 813 mmol) was added. After stirring for 10 min, isopropyl iodide (74.4 mL, 744 mmol) was added dropwise at 0 to −10° C. After the addition, the reaction mixture was warmed to room temperature and stirred overnight. The crude solution was directly used for the next step without further workup or purification.

### Step 4: 3-Methyl-2-(3-(2-(trityloxy)ethoxy)isoxazol-5-yl)butanoic acid

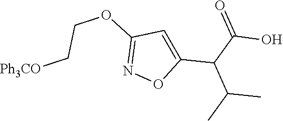

To a crude solution of methyl 3-methyl-2-(3-(2-(trityloxy)ethoxy)isoxazol-5-yl)butanoate (from Step 2) was added 1.25 N NaOH (aq.) and ethanol (500 mL). The resulting mixture was stirred for 1 h. The reaction mixture was concentrated, and the aqueous phase was adjusted to pH 3-5 with 4 N HCl (aq.). The solution was extracted with MTBE (3×1 L), and the combined organic phases were dried over MgSO₄ and concentrated. The residue was triturated with ethyl acetate (750 mL) to afford the title compound (227 g, 482 mmol, 71.3% yield over two steps). ¹H-NMR (400 MHz, DMSO-d6) δ 12.89 (s, 1H), 7.42-7.19 (m, 15H), 6.24 (s, 1H), 4.46-4.20 (m, 2H), 3.50 (d, J=8.7 Hz, 1H), 3.28-3.21 (m, 2H), 2.37-2.15 (m, 1H), 0.94 (d, J=6.7 Hz, 3H), 0.83 (d, J=6.7 Hz, 3H). LC-MS calc. for C₂₉H₂₉NO₅Na [M+Na]⁺m/z=494.2; Found 494.1.

### Step 5: 2-(3-(2-Hydroxyethoxy)isoxazol-5-yl)-3-methylbutanoic acid

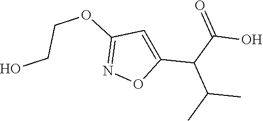 [SMILES: CC(C)C(C(=O)O)c1cc(OCCO)no1]

To a round bottom flask was added 3-methyl-2-(3-(2-(trityloxy)ethoxy)isoxazol-5-yl)butanoic acid (200 g, 424 mol.), THE (900 mL), and 4 N HCl (aq.) (500 mL). The reaction mixture was stirred for 4 h. The layers were separated, and the aqueous phase was extracted with MTBE (3×600 mL). To the combined organic phases was added 2 N NaOH (aq.) (1.5 L), and the mixture was stirred for 10 min. The layers were separated, and the aqueous layer was extracted with MTBE (2×500 mL). Then, the aqueous phase was adjusted to pH 1-2 with 2 N HCl (aq.) and extracted with MTBE (3×1 L). The combined organic phases were dried over MgSO₄ and concentrated to afford the title compound (76 g, 330 mmol, 78% yield) as a white solid. ¹H-NMR (400 MHz, DMSO-d6) δ 12.87 (s, 1H), 6.12 (s, 1H), 4.88 (s, 1H), 4.28-3.98 (m, 2H), 3.66 (d, J=3.8 Hz, 2H), 3.48 (d, J=8.7 Hz, 1H), 2.35-2.15 (m, 1H), 0.93 (d, J=6.7 Hz, 3H), 0.82 (d, J=6.7 Hz, 3H). LCMS calc. for C₁₀H₁₅NO₅Na [M+Na]⁺ m/z=252.1; Found 252.1.

### Step 6: (2S,4R)-4-((tert-Butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide

To a round bottom flask was added HATU (62 g, 160 mmol) and DCM (0.5 L). A solution of 2-(3-(2-hydroxyethoxy)isoxazol-5-yl)-3-methylbutanoic acid (31 g, 140 mmol), (2S,4R)-4-((tert-butyldimethylsilyl)oxy)-N—((S)-1-(4-(4-methylthiazol-5-yl)phenyl)ethyl)pyrrolidine-2-carboxamide (71.9 g, 162 mmol), and triethylamine (56.3 mL, 404 mmol) in DCM (0.8 L) was added over 15 min. The resulting solution was stirred for 10 min. The reaction was quenched with water (1.2 L), and the layers were separated. The aqueous layer was extracted with DCM (800 mL), and the combined organic layers were concentrated. The crude material was dissolved in MTBE (1 L) and washed with water (1 L) and 5% citric acid (aq.) (3-4×1.2 L). The organic layer was then washed with sat. NaHCO₃ (aq.) (500 mL), concentrated, and purified by SFC to give two diastereomers: “diastereomer A” and the desired “diastereomer B,” which was the title compound (58 g, 88 mmol, 63% yield) as a light yellow oil. ¹H-NMR (400 MHz, CDCl₃) δ 8.67 (s, 1H), 7.50-7.32 (m, 5H), 5.92 (d, J=0.7 Hz, 1H), 5.16-4.99 (m, 1H), 4.69-4.53 (m, 2H), 4.34 (tdd, J=3.4, 2.7, 0.7 Hz, 2H), 4.01-3.88 (m, 2H), 3.74 (dd, J=10.1, 5.5 Hz, 1H), 3.47 (d, J=10.0 Hz, 1H), 3.45-3.40 (m, 1H), 2.52 (d, J=0.7 Hz, 3H), 2.47 (d, J=13.0 Hz, 2H), 1.91-1.81 (m, 1H), 1.48 (d, J=6.9 Hz, 3H), 1.03 (d, J=6.5 Hz, 3H), 0.91 (d, J=6.7 Hz, 3H), 0.81 (d, J=0.7 Hz, 9H), 0.04 (d, J=7.7 Hz, 6H). LC-MS calc. for C₃₃H₄₉N₄O₆SSi [M+H]⁺: m/z=657.3; Found 657.1.

### Intermediate 3. tert-Butyl (6R,6aS)-2-chloro-6-methyl-5,6,6a,7,9,10-hexahydro-8H-pyrazino[1′,2′:4,5]pyrazino[2,3-c]pyridazine-8-carboxylate

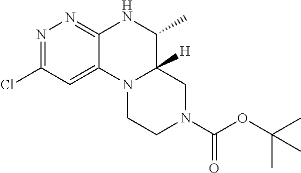 [SMILES: C[C@H]1Nc2nnc(Cl)cc2N2CCN(C(=O)OC(C)(C)C)C[C@@H]12]

### and

### Intermediate 4. tert-Butyl (6S,6aS)-2-chloro-6-methyl-5,6,6a,7,9,10-hexahydro-8H-pyrazino[1′,2′:4,5]pyrazino[2,3-c]pyridazine-8-carboxylate

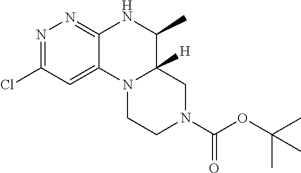 [SMILES: C[C@@H]1Nc2nnc(Cl)cc2N2CCN(C(=O)OC(C)(C)C)C[C@@H]12]

### Step 1: tert-Butyl (S)-4-(3,6-dichloropyridazin-4-yl)-3-(hydroxymethyl)piperazine-1-carboxylate

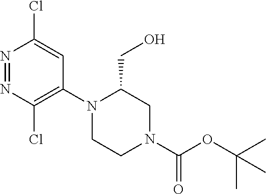 [SMILES: CC(C)(C)OC(=O)N1CCN(c2cc(Cl)nnc2Cl)[C@H](CO)C1]

To tert-butyl (S)-3-(hydroxymethyl)piperazine-1-carboxylate (2.90 g, 13.4 mmol) and 3,4,6-trichloropyridazine (2.00 g, 10.9 mmol) in DMSO (20 mL) was added N,N-diisopropylethylamine (2.85 mL, 16.4 mmol). The reaction was stirred at 80° C. overnight then allowed to cool to rt. The reaction was diluted with water (˜50 mL) and stirred vigorously and sonicated. The solid was filtered and washed with water and hexanes to afford the title compound (3.21 g, 81%) as a beige solid. LCMS calc. for C₁₄H₂₁Cl₂N₄O₃ [M+H]⁺: m/z=363.1; Found: 363.0.

### Step 2: tert-Butyl (S)-4-(3,6-dichloropyridazin-4-yl)-3-formylpiperazine-1-carboxylate

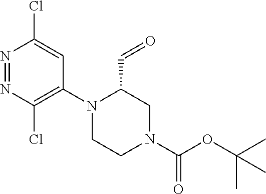 [SMILES: CC(C)(C)OC(=O)N1CCN(c2cc(Cl)nnc2Cl)[C@H](C=O)C1]

To a solution of tert-butyl (S)-4-(3,6-dichloropyridazin-4-yl)-3-(hydroxymethyl)piperazine-1-carboxylate (1.50 g, 4.13 mmol) in DCM (30 mL) at 0° C. was added Dess-Martin periodinane (2.45 g, 5.78 mmol), portion-wise, over 2 min. The reaction was stirred at rt for 50 min then concentrated to -1/4 volume and purified via SiO₂ FCC (0-60% EtOAc in hexanes) to afford the title compound (1.107 g, 74%) as a yellow solid. LCMS calc. for C₁₄H₁₉Cl₂N₄O₃ [M+H]⁺: m/z=361.1; Found: 360.9.

### Step 3: tert-Butyl (3S)-4-(3,6-dichloropyridazin-4-yl)-3-(1-hydroxyethyl)-3-methylpiperazine-1-carboxylate

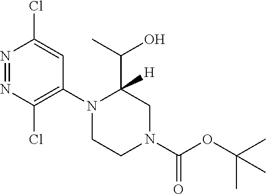 [SMILES: CC(O)[C@@H]1CN(C(=O)OC(C)(C)C)CCN1c1cc(Cl)nnc1Cl]

To a solution of tert-butyl (S)-4-(3,6-dichloropyridazin-4-yl)-3-formylpiperazine-1-carboxylate (1.10 g, 3.04 mmol) in THE (35 mL) at 0° C. was added MeMgBr (7.29 mL, 9.11 mmol) dropwise. The reaction was stirred at 0° C. for 35 min then quenched slowly with sat. NH₄Cl (aq.). The reaction was poured into water and EtOAc was added. The layers were separated, and the aqueous layer was extracted once with EtOAc. The combined organic layers were dried with MgSO₄, filtered, and concentrated. The crude residue was purified via SiO₂ FCC (0-60% EtOAc in hexanes) to afford tert-butyl (3S)-4-(3,6-dichloropyridazin-4-yl)-3-(1-hydroxyethyl)-3-methylpiperazine-1-carboxylate (783 mg, 68%) as a yellow solid. LCMS calc. for C₁₅H₂₃Cl₂N₄O₃ [M+H]⁺: m/z=377.1; Found: 377.0.

### Step 4: tert-Butyl (6R,6aS)-2-chloro-6-methyl-5,6,6a,7,9,10-hexahydro-8H-pyrazino[1′,2′:4,5]pyrazino[2,3-c]pyridazine-8-carboxylate

### and

### tert-butyl (6S,6aS)-2-chloro-6-methyl-5,6,6a,7,9,10-hexahydro-8H-pyrazino[1′,2′:4,5]pyrazino[2,3-c]pyridazine-8-carboxylate

To a solution of tert-butyl (3S)-4-(3,6-dichloropyridazin-4-yl)-3-(1-hydroxyethyl)-3-methylpiperazine-1-carboxylate (783 mg, 2.08 mmol) and PPh₃ (817 mg, 3.11 mmol) in THF (20 mL) at 0° C. was added diisopropyl azodicarboxylate (0.613 mL, 3.11 mmol), followed by DPPA (1.12 mL, 5.19 mmol). The reaction was stirred at rt for 40 min then treated with additional PPh₃ (1.63 g, 6.23 mmol). The reaction was stirred at 60° C. for 1 h, after which N,N-diisopropylethylamine (1.81 mL, 10.4 mmol) and water (3 mL) were added. The reaction was stirred at 60° C. overnight then allowed to cool to rt. The resulting residue was purified via SiO₂ FCC (0-10% MeOH in DCM) then chiral semi-prep (Lux IA3, starting at 50% 1:1 IPA:MeOH at 30 mL/min, -125 bar) to afford the title compound (Intermediate 3; 104 mg, 15%); LCMS calc. for C₁₅H₂₃ClN₅O₂[M+H]⁺: m/z=340.2; Found: 340.0; chiral semi-prep retention time: 3.26 min; ¹H NMR (400 MHz, DMSO) δ 7.24 (s, 1H), 6.74 (s, 1H), 3.99-3.87 (m, 2H), 3.87-3.80 (m, 1H), 3.68-3.58 (m, 1H), 3.01-2.84 (m, 2H), 2.75-2.54 (m, 1H), 1.41 (s, 9H), 1.12 (d, J=6.5 Hz, 3H), note: significant overlap observed between one proton peak (6 3.28-3.35 (m)) and residual water peak; and the title compound (Intermediate 4; 111 mg, 16%). LCMS calc. for C₁₅H₂₃ClN₅O₂[M+H]⁺: m/z=340.2; Found: 340.1; chiral semi-prep retention time: 4.86 min; ¹H NMR (400 MHz, DMSO) δ 7.31 (s, 1H), 6.69 (s, 1H), 4.15-3.98 (m, 1H), 3.98-3.86 (m, 1H), 3.85-3.74 (m, 1H), 3.32-3.22 (m, 1H), 3.05-2.89 (m, 1H), 2.89-2.74 (m, 2H), 2.72-2.53 (m, 1H), 1.42 (s, 9H), 1.19 (d, J=6.2 Hz, 3H).

### Intermediate 5. tert-Butyl (6S,6aR)-2-chloro-6-methyl-5,6,6a,7,9,10-hexahydro-8H-pyrazino[1′,2′:4,5]pyrazino[2,3-c]pyridazine-8-carboxylate

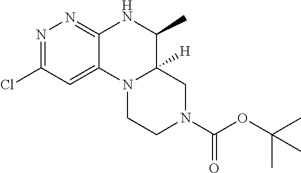 [SMILES: C[C@@H]1Nc2nnc(Cl)cc2N2CCN(C(=O)OC(C)(C)C)C[C@H]12]

### and

### Intermediate 6. tert-Butyl (6R,6aR)-2-chloro-6-methyl-5,6,6a,7,9,10-hexahydro-8H-pyrazino[1′,2′:4,5]pyrazino[2,3-c]pyridazine-8-carboxylate

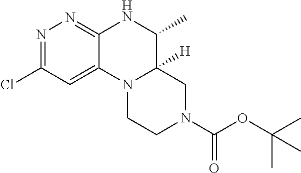 [SMILES: C[C@H]1Nc2nnc(Cl)cc2N2CCN(C(=O)OC(C)(C)C)C[C@H]12]

The title compounds (Intermediate 5 and Intermediate 6) were prepared using procedures analogous to those used to synthesize Intermediate 3 and Intermediate 4, starting with tert-butyl (R)-3-(hydroxymethyl)piperazine-1-carboxylate instead of tert-butyl (S)-3-(hydroxymethyl)piperazine-1-carboxylate. Intermediate 5: LCMS calc. for C₁₅H₂₃ClN₅O₂ [M+H]⁺: m/z=340.2; Found: 340.1. Retention time: 1.020 min (CSH Fluoro-Phenyl, 2.1×50 mm, 1.7 μm, 2-100% MeCN (0.025% TFA (aq.)) over 1.8 min at 1 mL/min). Intermediate 6: LCMS calc. for C₁₅H₂₃ClN₅O₂[M+H]⁺: m/z=340.2; Found: 340.0. Retention time: 1.048 min (CSH Fluoro-Phenyl, 2.1×50 mm, 1.7 μm, 2-100% MeCN (0.025% TFA (aq.)) over 1.8 min at 1 mL/min).

### Intermediate 7. 2-((6R,6aS)-6-Methyl-8-(piperidin-4-yl)-6,6a,7,8,9,10-hexahydro-5H-pyrazino[1′,2′:4,5]pyrazino[2,3-c]pyridazin-2-yl)phenol

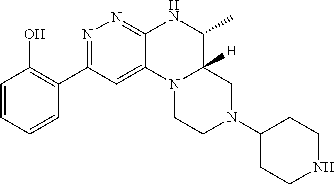 [SMILES: C[C@H]1Nc2nnc(-c3ccccc3O)cc2N2CCN(C3CCNCC3)C[C@@H]12]

### Step 1: 2-((6R,6aS)-6-Methyl-6,6a,7,8,9,10-hexahydro-5H-pyrazino[1′,2′:4,5]pyrazino [2,3-c]pyridazin-2-yl)phenol

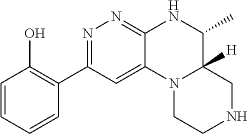 [SMILES: C[C@H]1Nc2nnc(-c3ccccc3O)cc2N2CCNC[C@@H]12]

A mixture of tert-butyl (6R,6aS)-2-chloro-6-methyl-5,6,6a,7,9,10-hexahydro-8H-pyrazino[1′,2′:4,5]pyrazino[2,3-c]pyridazine-8-carboxylate (Intermediate 3; 30.0 mg, 88.3 μmol), 2-hydroxyphenylboronic acid (24.4 mg, 0.177 mmol), XPhos Pd G2 (6.95 mg, 8.83 μmol) and K₃PO₄ (56.2 mg, 0.265 mmol) in 1,4-dioxane (1.8 mL) and water (0.2 mL) was stirred at 100° C. for 2 h. The reaction was cooled to rt, concentrated and the crude residue was purified via prep-HPLC on a C18 column (10-40% MeCN/0.05% TFA (aq.)). The fraction containing the desired product was concentrated and the resulting residue was treated with TFA (2 mL), concentrated, and lyophilized to afford the TFA salt of the title compound (56.1 mg, 99%) as a brown solid. LCMS calc. for C₁₆H₂₀N₅O [M+H]⁺m/z=298.2; Found 298.3.

### Step 2: 2-((6R,6aS)-6-Methyl-8-(piperidin-4-yl)-6,6a,7,8,9,10-hexahydro-5H-pyrazino[1′,2′:4,5]pyrazino[2,3-c]pyridazin-2-yl)phenol

To a solution of 2-((6R,6aS)-6-methyl-6,6a,7,8,9,10-hexahydro-5H-pyrazino[1′,2′:4,5]pyrazino[2,3-c]pyridazin-2-yl)phenol TFA salt (45.9 mg, 0.0718 mmol), tert-butyl 4-oxopiperidine-1-carboxylate (28.6 mg, 0.144 mmol) and N,N-diisopropylethylamine (62.5 μL, 0.359 mmol) in DCE (2.0 mL) was added sodium triacetoxyborohydride (30.4 mg, 0.144 mmol). The reaction was stirred at room temperature for 16 h then treated with sat. NaHCO₃ (aq.) (3.0 mL) and DCM (3.0 mL). The layers were separated, and the aqueous phase was extracted with DCM (3×3 mL). The combined organic layers were dried over Na₂SO₄ and concentrated. The crude material was purified via prep-HPLC on a C18 column (10-40% MeCN/0.05% TFA (aq.)), after which fractions containing the desired product were concentrated. The resulting residue was treated with TFA (2 mL) then concentrated to give the TFA salt of the title compound (15.7 mg, 26%) as a brown solid. LCMS calc. for C₂₁H₂₉N60 [M+H]⁺m/z=381.2; Found 381.4.

### Intermediate 8. 2-((6S,6aS)-6-Methyl-8-(piperidin-4-yl)-6,6a,7,8,9,10-hexahydro-5H-pyrazino[1′,2′:4,5]pyrazino[2,3-c]pyridazin-2-yl)phenol

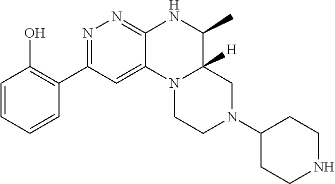 [SMILES: C[C@@H]1Nc2nnc(-c3ccccc3O)cc2N2CCN(C3CCNCC3)C[C@@H]12]

### Step 1: tert-Butyl (6S,6aS)-2-(2-hydroxyphenyl)-6-methyl-5,6,6a,7,9,10-hexahydro-8H-pyrazino[1′,2′:4,5]pyrazino[2,3-c]pyridazine-8-carboxylate

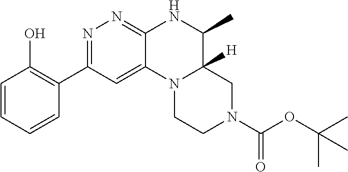 [SMILES: C[C@@H]1Nc2nnc(-c3ccccc3O)cc2N2CCN(C(=O)OC(C)(C)C)C[C@@H]12]

To a solution of tert-butyl (6S,6aS)-2-chloro-6-methyl-5,6,6a,7,9,10-hexahydro-8H-pyrazino[1′,2′:4,5]pyrazino[2,3-c]pyridazine-8-carboxylate (Intermediate 4; 51.5 mg, 0.152 mmol), 2-hydroxyphenylboronic acid (20.9 mg, 0.152 mmol) in 1,4-dioxane (5.0 mL) and water (0.25 mL) was added K₃PO₄ (96.5 mg, 0.455 mmol). The mixture was bubbled with N₂ gas for 5 min, followed by the addition of XPhos Pd G2 (11.9 mg, 15.2 μmol). The mixture was bubbled for another 5 min and stirred at 90° C. for 12 h. The reaction mixture was cooled to rt and concentrated before addition of H₂O (3.0 mL). The resulting mixture was extracted with ethyl acetate (3×3 mL). The combined organic layers were dried over Na₂SO₄, filtered and concentrated. The crude residue was purified by prep-HPLC on a C18 column (5-60% MeCN/0.05% formic acid (aq.)) to afford the formate salt of the title compound (60.0 mg, 89.0% yield). LCMS calc. for C₂₁H₂₈N₅O₃ [M+H]⁺m/z=398.2; Found 398.3.

### Step 2: 2-((6S,6aS)-6-Methyl-6,6a,7,8,9,10-hexahydro-5H-pyrazino[1′,2′:4,5]pyrazino [2,3-c]pyridazin-2-yl)phenol

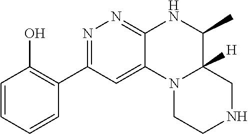 [SMILES: C[C@@H]1Nc2nnc(-c3ccccc3O)cc2N2CCNC[C@@H]12]

To a solution of tert-butyl (6S,6aS)-2-(2-hydroxyphenyl)-6-methyl-5,6,6a,7,9,10-hexahydro-8H-pyrazino[1′,2′:4,5]pyrazino[2,3-c]pyridazine-8-carboxylate formate salt (60.0 mg, 0.135 mmol) in DCM (3.0 mL) was added TFA (1.0 mL). The mixture was stirred at rt for 3 h. The mixture was concentrated and the resulting crude residue containing the TFA salt of the title compound was used without further purification. LCMS calc. for C₁₆H₂₀N₅O [M+H]⁺m/z=298.2; Found 298.3.

### Step 3: tert-Butyl 4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidine-1-carboxylate

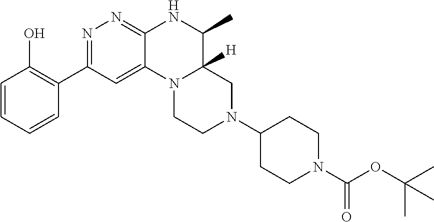 [SMILES: C[C@@H]1Nc2nnc(-c3ccccc3O)cc2N2CCN(C3CCN(C(=O)OC(C)(C)C)CC3)C[C@@H]12]

To a solution of crude 2-((6S,6aS)-6-methyl-6,6a,7,8,9,10-hexahydro-5H-pyrazino[1′,2′:4,5]pyrazino[2,3-c]pyridazin-2-yl)phenol TFA salt in DCE (3.0 mL) and DMSO (1.0 mL) was added N,N-diisopropylethylamine (98 μl, 0.56 mmol), followed by the addition of tert-butyl 4-oxopiperidine-1-carboxylate (75 mg, 0.37 mmol) and sodium triacetoxyborohydride (120 mg, 0.56 mmol). The mixture was stirred at rt for 12 h. The crude was purified by prep-HPLC on a C18 column (5-50% MeCN/0.05% formic acid (aq.)) to afford the formate salt of the title compound (50 mg, 70% yield over 2 steps). LCMS calc. for C₂₆H₃₇N₆O₃ [M+H]⁺m/z=481.3; Found 481.3.

### Step 4: 2-((6S,6aS)-6-Methyl-8-(piperidin-4-yl)-6,6a,7,8,9,10-hexahydro-5H-pyrazino[1′,2′:4,5]pyrazino[2,3-c]pyridazin-2-yl)phenol

To a solution of tert-butyl 4-((6S,6aS)-2-(2-hydroxyphenyl)-6-methyl-5,6,6a,7,9,10-hexahydro-8H-pyrazino[1′,2′:4,5]pyrazino[2,3-c]pyridazin-8-yl)piperidine-1-carboxylate (50.0 mg, 0.104 mmol) in DCM (2.0 mL) was added TFA (0.50 mL). The mixture was stirred at rt for 3 h. The reaction mixture was concentrated and the resulting residue containing the TFA salt of the title compound was used in the next step without further purification. LCMS calc. for C₂₁H₂₉N₆O [M+H]⁺m/z=381.2; Found 381.3.

### Intermediate 10. 2-((6R,6aR)-6-Methyl-8-(piperidin-4-yl)-6,6a,7,8,9,10-hexahydro-5H-pyrazino[1′,2′:4,5]pyrazino[2,3-c]pyridazin-2-yl)phenol

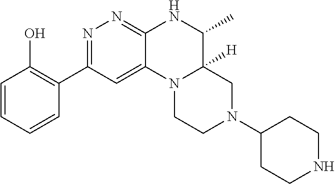 [SMILES: C[C@H]1Nc2nnc(-c3ccccc3O)cc2N2CCN(C3CCNCC3)C[C@H]12]

### Step 1: tert-Butyl (6R,6aR)-2-(2-hydroxyphenyl)-6-methyl-5,6,6a,7,9,10-hexahydro-8H-pyrazino[1′,2′:4,5]pyrazino[2,3-c]pyridazine-8-carboxylate

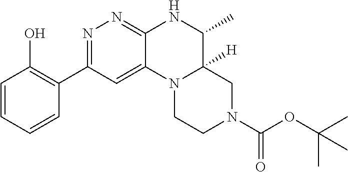 [SMILES: C[C@H]1Nc2nnc(-c3ccccc3O)cc2N2CCN(C(=O)OC(C)(C)C)C[C@H]12]

A mixture of tert-butyl (6R,6aR)-2-chloro-6-methyl-5,6,6a,7,9,10-hexahydro-8H-pyrazino[1′,2′:4,5]pyrazino[2,3-c]pyridazine-8-carboxylate (29.0 mg, 0.0853 mmol), 2-hydroxyphenylboronic acid (35.3 mg, 0.256 mmol), XPhos Pd G2 (6.71 mg, 0.0853 mmol), and K₃PO₄ (72.5 mg, 0.341 mmol) in 1,4-dioxane (0.83 mL) and water (45 μL) was sparged with N₂. The reaction mixture was stirred at 95° C. for 2 h after which the mixture was diluted with EtOAc and water. The layers were separated, and the aqueous phase was extracted with EtOAc (1×). The combined organic layers were dried with Na₂SO₄ and concentrated. The crude residue was purified via prep-HPLC (10-30% MeCN/0.02% formic acid (aq.)). The fractions containing desired product were combined and basified to pH 8 with 5% NaHCO₃ (aq.), and the acetonitrile was removed under reduced pressure. The resulting aqueous layer was extracted with EtOAc (3×) and the combined organic layers were dried with Na₂SO₄ then concentrated to afford the free-base of the title compound (29.0 mg, 79.5%). LCMS calc. for C₂₁H₂₈N₅O₃ [M+H]⁺m/z=398.2; Found 398.1.

### Step 2: 2-((6R,6aR)-6-methyl-6,6a,7,8,9,10-hexahydro-5H-pyrazino[1′,2′:4,5]pyrazino [2,3-c]pyridazin-2-yl)phenol

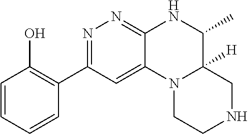 [SMILES: C[C@H]1Nc2nnc(-c3ccccc3O)cc2N2CCNC[C@H]12]

To tert-butyl (6R,6aR)-2-(2-hydroxyphenyl)-6-methyl-5,6,6a,7,9,10-hexahydro-8H-pyrazino[1′,2′:4,5]pyrazino[2,3-c]pyridazine-8-carboxylate (29.0 mg, 0.0730 mmol) was added HCl (4.0 M in dioxane; 365 μL, 1.46 mmol). The reaction was stirred at rt for 1 h then concentrated to dryness to give the HCl salt of the title compound (27.0 mg, 80.4%) as a white solid, which was used without further purification. LCMS calc. for C₁₆H₂₀N50 [M+H]⁺m/z=298.2; Found 298.0.

### Step 3: tert-Butyl 4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidine-1-carboxylate

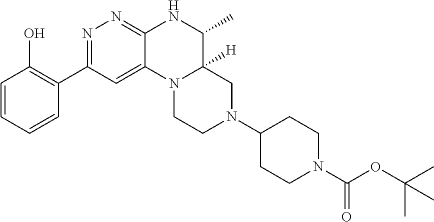 [SMILES: C[C@H]1Nc2nnc(-c3ccccc3O)cc2N2CCN(C3CCN(C(=O)OC(C)(C)C)CC3)C[C@H]12]

2-((6R,6aR)-6-methyl-6,6a,7,8,9,10-hexahydro-5H-pyrazino[1′,2′:4,5]pyrazino[2,3-c]pyridazin-2-yl)phenol HCl salt (27.0 mg, 0.0730 mmol), tert-butyl 4-oxopiperidine-1-carboxylate (72.6 mg, 0.365 mmol), and N,N-diisopropylethylamine (63.5 μL, 0.365 mmol) in DCE (225 μL) were stirred for 30 min before addition of sodium triacetoxyborohydride (23.2 mg, 0.109 mmol). The reaction was stirred at rt overnight then concentrated to dryness. The crude residue was taken up in DMSO and purified via prep-HPLC on a C18 column (10-40% MeCN/0.02% TFA (aq.)). The fractions containing the desired product were combined and basified to pH 8 with 5% NaHCO₃, and the acetonitrile was removed under reduced pressure. The resulting aqueous layer was extracted with EtOAc (3×), and the combined organic layers were dried with Na₂SO₄ then concentrated to afford the free-base of the title compound (18.0 mg, 49.9%). LCMS calc. for C₂₆H₃₇N₆O₃ [M+H]⁺m/z=481.3; Found 481.1.

### Step 4: 2-((6R,6aR)-6-Methyl-8-(piperidin-4-yl)-6,6a,7,8,9,10-hexahydro-5H-pyrazino[1′,2′:4,5]pyrazino[2,3-c]pyridazin-2-yl)phenol

To tert-butyl 4-((6R,6aR)-2-(2-hydroxyphenyl)-6-methyl-5,6,6a,7,9,10-hexahydro-8H-pyrazino[1′,2′:4,5]pyrazino[2,3-c]pyridazin-8-yl)piperidine-1-carboxylate (18.0 mg, 0.0375 mmol) was added HCl (4.0 M in dioxane; 190 μL, 0.749 mmol). The reaction was stirred at rt for 1 h then concentrated to dryness to give the HCl salt of the title compound (17.0 mg, 99.1%) as a white solid. LCMS calc. for C₂₁H₂₉N₆O [M+H]⁺m/z=381.2; Found 381.2.

### Example 3. Synthesis of Compound 2.38—4-((S)-2-((S)-2-(6-(2,5-Dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

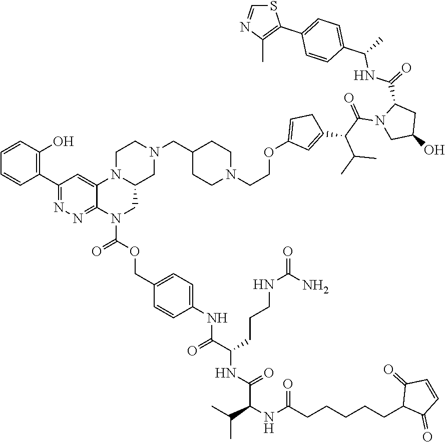 [SMILES: Cc1ncsc1-c1ccc([C@H](C)NC(=O)[C@@H]2C[C@@H](O)CN2C(=O)[C@@H](C2=CC(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCC7C(=O)C=CC7=O)C(C)C)cc6)C[C@H]5C4)CC3)=CC2)C(C)C)cc1]

### Step 1: tert-Butyl (S)-4-((2-chloro-5,6,6a,7,9,10-hexahydro-8H-pyrazino[1′,2′:4,5]pyrazino[2,3-c]pyridazin-8-yl)methyl)piperidine-1-carboxylate

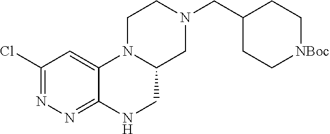 [SMILES: CC(C)(C)OC(=O)N1CCC(CN2CCN3c4cc(Cl)nnc4NC[C@H]3C2)CC1]

To a suspension of (R)-2-chloro-6,6a,7,8,9,10-hexahydro-5H-pyrazino[1′,2′:4,5]pyrazino[2,3-c]pyridazine tetrahydrochloride (2.79 g, 7.51 mmol) in DCE (80 mL) was added triethylamine (5.23 mL, 37.6 mmol), and the mixture was sonicated for 5 min. 1-Boc-Piperidine-4-carboxaldehyde (2.4 g, 11 mmol) and then sodium triacetoxyborohydride (3.2 g, 15 mmol) were added. The mixture was stirred for 30 min. To the mixture was added sat. NaHCO₃ (aq.) (25 mL) and water (25 mL). The organic phase was separated, and the aqueous phase was extracted with DCM (3×50 mL). The combined organic phases were dried over Na₂SO₄, concentrated, and purified by silica gel chromatography (0%-20% MeOH/DCM) to give the title compound (3.1 g, 7.3 mmol, 98% yield), a colorless solid. LCMS calc. for C₂₀H₃₂ClN₆O₂[M+H]⁺m/z=423.2; Found 423.3.

### Step 2: tert-Butyl (S)-4-((2-(2-(methoxymethoxy)phenyl)-5,6,6a,7,9,10-hexahydro-8H-pyrazino[1′,2′:4,5]pyrazino[2,3-c]pyridazin-8-yl)methyl)piperidine-1-carboxylate

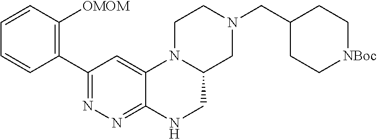

A solution of tert-butyl (S)-4-((2-chloro-5,6,6a,7,9,10-hexahydro-8H-pyrazino[1′,2′:4,5]pyrazino[2,3-c]pyridazin-8-yl)methyl)piperidine-1-carboxylate (2.78 g, 6.57 mmol), 2-(methoxymethoxy)phenylboronic acid (2.39 g, 13.2 mmol), potassium carbonate (2.73 g, 19.7 mmol) and [1,1′-bis(diphenylphosphino)ferrocene]dichloropalladium(II) (385 mg, 0.526 mmol) in 1,4-dioxane (40 mL) and water (10 mL) was heated to 100° C. under N₂ atmosphere for 2 h. The mixture was cooled to room temperature, concentrated, and diluted between DCM (50 mL) and water (50 mL). The mixture was filtered, and the organic phase was separated. The aqueous phase was extracted with DCM (3×50 mL). The combined organic phases were dried over Na₂SO₄, concentrated, and purified by silica gel chromatography (0%-20% MeOH/DCM) to give the title compound (3.0 g, 5.7 mmol, 87% yield), a brown solid. LCMS calc. for C₂₈H₄₁N₆O₄ [M+H]⁺m/z=525.3; Found 525.4.

### Step 3: (S)-2-(2-(Methoxymethoxy)phenyl)-8-(piperidin-4-ylmethyl)-6,6a,7,8,9,10-hexahydro-5H-pyrazino[1′,2′:4,5]pyrazino[2,3-c]pyridazine

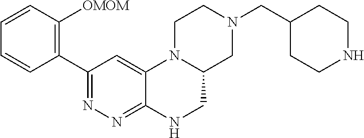

To a solution of tert-butyl (S)-4-((2-(2-(methoxymethoxy)phenyl)-5,6,6a,7,9,10-hexahydro-8H-pyrazino[1′,2′:4,5]pyrazino[2,3-c]pyridazin-8-yl)methyl)piperidine-1-carboxylate (3.0 g, 5.7 mmol) in DCM (50 mL) was added 2,6-lutidine (4.31 mL, 37.2 mmol). The mixture was cooled to 0° C., trimethylsilyl trifluoromethanesulfonate (6.22 mL, 34.3 mmol) was added dropwise. The mixture was warmed up to room temperature and stirred for 16 h. To the mixture was added MeOH (20 mL). The mixture was concentrated and purified by prep-HPLC on C₁₈ column (5-30% MeCN/0.05% TFA (aq.)), and the desired fractions were concentrated and partitioned with 2 N NaOH (50 mL) and DCM (50 mL). The organic phase was separated, and the aqueous phase was extracted with DCM (3×50 mL). The combined organic phases were dried over Na₂SO₄ and concentrated to give the title compound (1.7 g, 4.0 mmol, 70% yield), a beige solid. LCMS calc. for C₂₃H₃₃N₆O₂ [M+H]⁺m/z=425.3; Found 425.3.

### Step 4: (2S,4R)-4-((tert-Butyldimethylsilyl)oxy)-1-((R)-2-(3-(2-(4-(((S)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

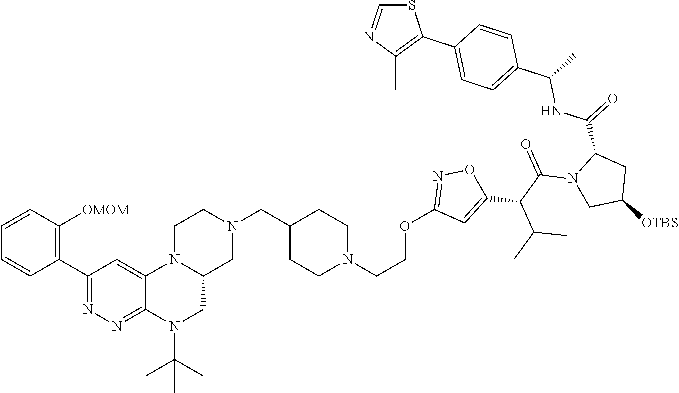

To a solution of (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide (1.2 g, 1.8 mmol) in MeCN (15 mL) was added 2-iodoxybenzoic acid (873 mg, 3.12 mmol). The resulting mixture was stirred at 80° C. for 1 h. The mixture was cooled to room temperature, filtered, concentrated, and redissolved in DCE (20 mL). To the mixture was added (S)-2-(2-(methoxymethoxy)phenyl)-8-(piperidin-4-ylmethyl)-6,6a,7,8,9,10-hexahydro-5H-pyrazino[1′,2′:4,5]pyrazino[2,3-c]pyridazine (781 mg, 1.84 mmol) and sodium triacetoxyborohydride (585 mg, 2.76 mmol). The mixture was stirred for 16 h. To the mixture was added saturated NaHCO₃ (aq.) (10 mL). The organic phase was separated, and the aqueous phase was extracted with DCM (3×10 mL). The combined organic phases were dried over Na₂SO₄, concentrated, and purified by silica gel chromatography (5%-15% MeOH/DCM) to give the title compound (1.5 g, 1.4 mmol, 77% yield), a brown solid. LCMS calc. for C₅₆H₇₉N₁₀O₇SSi [M+H]⁺m/z=1063.6; Found 1063.5.

### Step 5: 4—((S)-2-((S)-2-(6-(2,5-Dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate

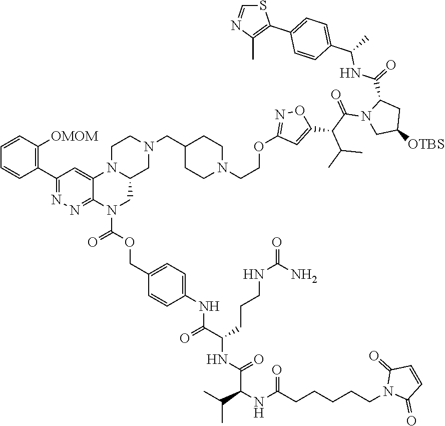

To a solution of (2S,4R)-4-((tert-butyldimethylsilyl)oxy)-1-((R)-2-(3-(2-(4-(((S)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (220 mg, 0.21 mmol) and Mc-Val-Cit-PABC-PNP (230 mg, 0.31 mmol, AmBeed, CAS: 159857-81-5) in DMF (2 mL) was added 2,6-lutidine (480 μL, 4.12 mmol). The mixture was stirred for 2 d. The mixture was diluted with DMSO and purified by prep-HPLC on C18 column (5-60% MeCN/0.05% TFA (aq.)) to afford the title compound (230 mg, 0.14 mmol, 67% yield), a colorless solid. LCMS calc. for C₈₅H₁₁₇N₁₆O₁₅SSi [M+H]⁺m/z=1661.8; Found 1661.4.

### Step 6: 4—((S)-2-((S)-2-(6-(2,5-Dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

To 4-((S)-2-((S)-2-(6-(2,5-dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate (230 mg, 0.14 mmol) was added IPA/conc. HCl (aq,) (1:1, 2 mL). The mixture was stirred for 5 min, diluted with water, and purified by prep-HPLC on C₁₈ column (5-40% MeCN/0.05% TFA (aq.)) to afford the title compound as a TFA salt (100 mg, 0.067 mmol, 48% yield), a colorless solid. LCMS calc. for C₇₇H₉₉N₁₆O₁₄S [M+H]⁺m/z=1503.7; Found 1503.6.

### Example 4. Synthesis of Compound 2.15—4-((S)-2-((S)-2-(6-(2,5-Dioxo-2,5-dihydro-1H-pyrrol-1-yl)hexanamido)-3-methylbutanamido)-5-ureidopentanamido)-3-methylbenzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

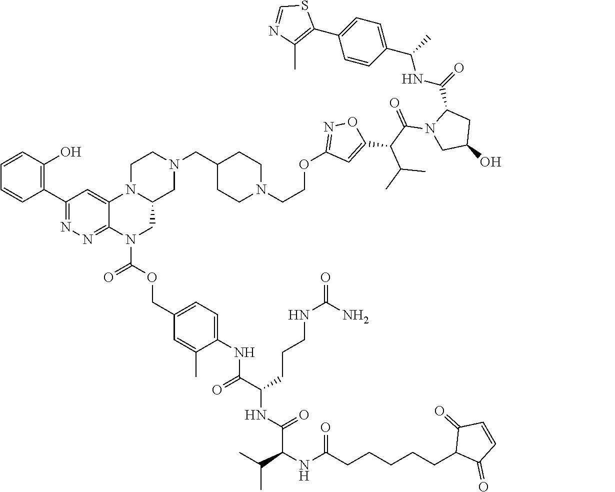 [SMILES: Cc1cc(COC(=O)N2C[C@H]3CN(CC4CCN(CCOc5cc([C@H](C(=O)N6C[C@H](O)C[C@H]6C(=O)N[C@@H](C)c6ccc(-c7scnc7C)cc6)C(C)C)on5)CC4)CCN3c3cc(-c4ccccc4O)nnc32)ccc1NC(=O)[C@H](CCCNC(N)=O)NC(=O)[C@@H](NC(=O)CCCCCC1C(=O)C=CC1=O)C(C)C]

### Step 1: tert-Butyl ((S)-1-(((S)-1-((4-(hydroxymethyl)-2-methylphenyl)amino)-1-oxo-5-ureidopentan-2-yl)amino)-3-methyl-1-oxobutan-2-yl)carbamate

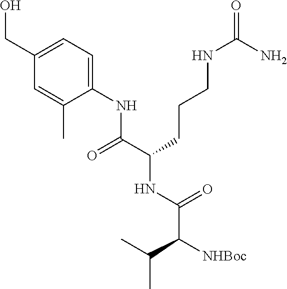 [SMILES: Cc1cc(CO)ccc1NC(=O)[C@H](CCCNC(N)=O)NC(=O)[C@@H](NC(=O)OC(C)(C)C)C(C)C]

To a solution of (S)-2-((S)-2-((tert-butoxycarbonyl)amino)-3-methylbutanamido)-5-ureidopentanoic acid (300 mg, 0.8 mmol) in DCM (3 mL) and methanol (1.5 mL) was added (4-amino-3-methylphenyl)methanol (130 mg, 0.96 mmol) and N-ethoxycarbonyl-2-ethoxy-1,2-dihydroquinoline (258 mg, 1.04 mmol). The mixture was stirred for 2 d. The mixture was concentrated. The resulting red solid was suspended in MTBE (20 mL), and the mixture was stirred for 1 h. The solid was collected by filtration, and the solid cake was washed with MTBE (50 mL). The collected solid was dried under vacuum to afford the title compound (328 mg, 0.665 mmol, 83.1% yield), a beige solid. LCMS calc. for C₂₄H₄₀N₅O₆ [M+H]⁺: m/z=494.3; Found 494.3.

### Step 2: (S)-2-((S)-2-amino-3-methylbutanamido)-N-(4-(hydroxymethyl)-2-methylphenyl)-5-ureidopentanamide

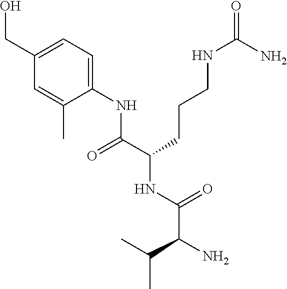 [SMILES: Cc1cc(CO)ccc1NC(=O)[C@H](CCCNC(N)=O)NC(=O)[C@@H](N)C(C)C]

To a solution of tert-butyl ((S)-1-(((S)-1-((4-(hydroxymethyl)-2-methylphenyl)amino)-1-oxo-5-ureidopentan-2-yl)amino)-3-methyl-1-oxobutan-2-yl)carbamate (155 mg, 0.314 mmol) in THE (1 mL) and IPA (1 mL) was added concentrated HCl (aq.) (1 mL). The mixture was stirred for 1 h. The reaction mixture was adjusted to pH 8-9 by dropwise addition of sat. NaHCO₃ (aq.). The solvent was removed under reduced pressure. The crude was purified by silica gel chromatography (0-100% MeOH/MTBE) to afford the title compound (115 mg, 0.292 mmol, 92.9% yield), a white solid. LCMS calc. for C₁₉H₃₂N₅O₄ [M+H]⁺: m/z=394.2; Found 394.2.

### Step 3: 6-(2,5-Dioxo-2,5-dihydro-1H-pyrrol-1-yl)-N—((S)-1-(((S)-1-((4-(hydroxymethyl)-2-methylphenyl)amino)-1-oxo-5-ureidopentan-2-yl)amino)-3-methyl-1-oxobutan-2-yl)hexanamide

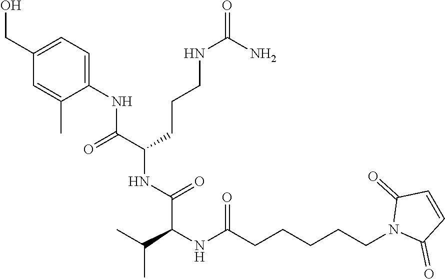 [SMILES: Cc1cc(CO)ccc1NC(=O)[C@H](CCCNC(N)=O)NC(=O)[C@@H](NC(=O)CCCCCN1C(=O)C=CC1=O)C(C)C]

To a solution of (S)-2-((S)-2-amino-3-methylbutanamido)-N-(4-(hydroxymethyl)-2-methylphenyl)-5-ureidopentanamide (115 mg, 0.292 mmol) in THE (2 mL) and water (2 mL) was added sodium bicarbonate (98.2 mg, 1.17 mmol) and 6-maleimidohexanoic acid N-hydroxysuccinimide ester (108 mg, 0.350 mmol). The mixture was stirred for 3 h. To the mixture was added water (5 mL), and the mixture was extracted with chloroform/IPA (3:1) (3×20 mL). The combined organic layers were dried over anhydrous Na₂SO₄ and concentrated. The crude was purified by silica gel chromatography (0-60% MeOH/MTBE) to afford the title compound (162 mg, 0.276 mmol, 94.5% yield), a beige solid. LCMS calc. for C₂₉H₄₃N₆O₇ [M+H]⁺: m/z=587.3; Found 587.4.

### Step 4: 4—((S)-2-((S)-2-(6-(2,5-Dioxo-2,5-dihydro-1H-pyrrol-1-yl)hexanamido)-3-methylbutanamido)-5-ureidopentanamido)-3-methylbenzyl (4-nitrophenyl) carbonate

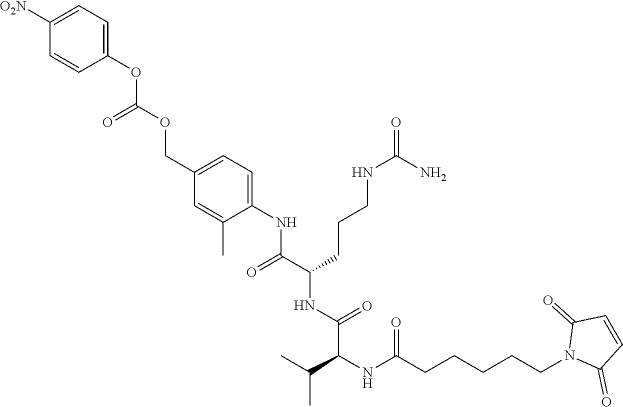 [SMILES: Cc1cc(COC(=O)Oc2ccc([N+](=O)[O-])cc2)ccc1NC(=O)[C@H](CCCNC(N)=O)NC(=O)[C@@H](NC(=O)CCCCCN1C(=O)C=CC1=O)C(C)C]

To a solution of 6-(2,5-dioxo-2,5-dihydro-1H-pyrrol-1-yl)-N—((S)-1-(((S)-1-((4-(hydroxymethyl)-2-methylphenyl)amino)-1-oxo-5-ureidopentan-2-yl)amino)-3-methyl-1-oxobutan-2-yl)hexanamide (100 mg, 0.17 mmol) in DMF (1.5 mL) was added NN-diisopropylethylamine (0.1 mL, 0.5 mmol) and 4-nitrophenyl carbonate (155 mg, 0.510 mmol). The resulting mixture was stirred overnight. The reaction mixture was purified by prep-HPLC on C18 column (10-60% MeCN/0.1% formic acid (aq.)) to afford the title compound (36 mg, 0.048 mmol, 28% yield), a white solid. LCMS calc. for C₃₆H₄₆N₇O₁₁ [M+H]⁺: m/z=752.3; Found 752.5.

### Step 5: 4—((S)-2-((S)-2-(6-(2,5-Dioxo-2,5-dihydro-1H-pyrrol-1-yl)hexanamido)-3-methylbutanamido)-5-ureidopentanamido)-3-methylbenzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

The title compound was synthesized by procedures analogous to those outlined in Example 3, Steps 5-6. LCMS calc. for C₇₈H₁₀₁N₁₆O₁₄S [M+H]⁺: m/z=1517.7; Found 1517.6.

### Example 5. Synthesis of Compound 2.40—4-((S)-2-((S)-2-(3-(2-(2,5-Dioxo-2,5-dihydro-1H-pyrrol-1-yl)ethoxy)propanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

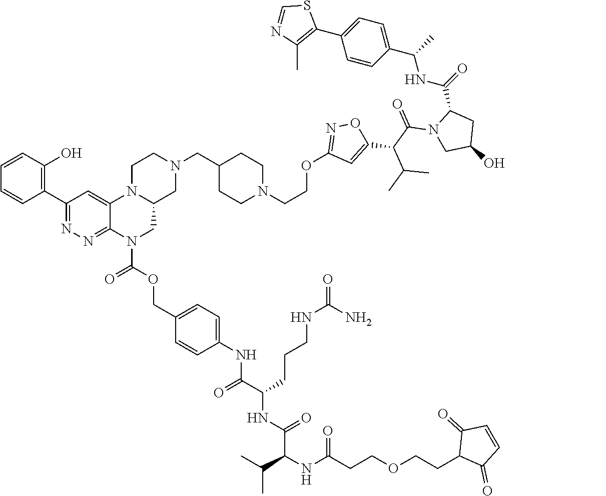 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCOCCC7C(=O)C=CC7=O)C(C)C)cc6)C[C@H]5C4)CC3)no2)C(C)C)cc1]

### Step 1: 4—((S)-2-((S)-2-Amino-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

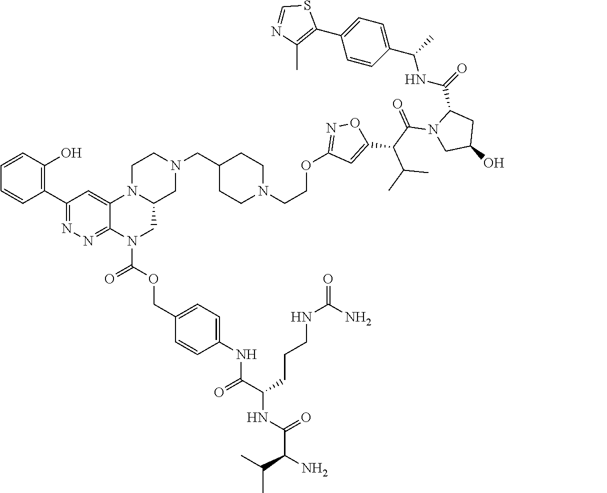 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](N)C(C)C)cc6)C[C@H]5C4)CC3)no2)C(C)C)cc1]

The title compound was synthesized by procedures analogous to those outlined in Example 3, Steps 5-6. LCMS calc. for C₆₇H₈₈N₁₅O₁₁S [M+H]⁺m/z=1310.6; Found 1310.7.

### Step 2: 4—((S)-2-((S)-2-(3-(2-(2,5-Dioxo-2,5-dihydro-1H-pyrrol-1-yl)ethoxy)propanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

To a solution of 2,5-dioxopyrrolidin-1-yl 3-(2-(2,5-dioxo-2,5-dihydro-1H-pyrrol-1-yl)ethoxy)propanoate (3.55 mg, 0.00931 mmol) and 4-((S)-2-((S)-2-amino-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate (7.5 mg, 0.0049 mmol) in DMF (0.5 mL) was added N,N-diisopropylethylamine (0.01 mL, 0.06 mmol). The reaction mixture was stirred at room temperature for 5 h. The reaction was diluted with MeOH and purified by prep-HPLC on C18 column (10-40% MeCN/0.05% TFA (aq.)) to give the title compound as a TFA salt (4.9 mg, 0.0032 mmol, 55% yield). LCMS calc. for C₇₆H₉₇N₁₆O₁₅S [M+H]⁺m/z=1505.7; Found 1505.7.

### Example 6. Synthesis of Compound 2.09—4-((S)-2-((S)-2-(6-(2,5-Dioxo-2,5-dihydro-1H-pyrrol-1-yl)hexanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-hydroxyphenyl)-6a-methyl-6a,7,8,9-tetrahydropyrrolo[1′,2′:4,5]pyrazino[2,3-c]pyridazine-5(6H)-carboxylate

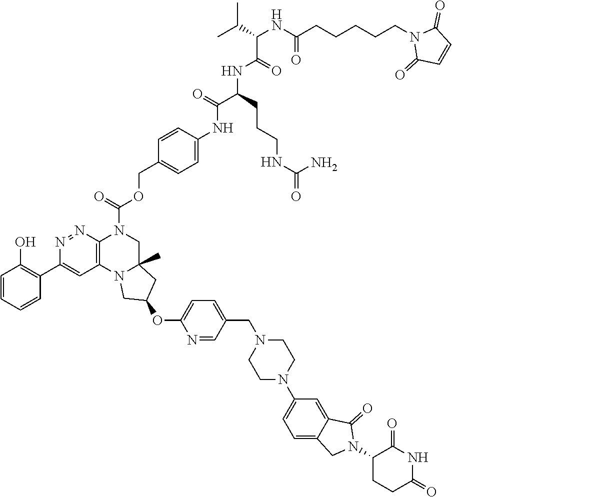 [SMILES: CC(C)[C@H](NC(=O)CCCCCN1C(=O)C=CC1=O)C(=O)N[C@@H](CCCNC(N)=O)C(=O)Nc1ccc(COC(=O)N2C[C@]3(C)C[C@@H](Oc4ccc(CN5CCN(c6ccc7c(c6)C(=O)N([C@H]6CCC(=O)NC6=O)C7)CC5)cn4)CN3c3cc(-c4ccccc4O)nnc32)cc1]

### Step 1: (6aS,8R)-8-((5-(1,3-Dioxolan-2-yl)pyridin-2-yl)oxy)-2-chloro-6a-methyl-5,6,6a,7,8,9-hexahydropyrrolo[1′,2′:4,5]pyrazino[2,3-c]pyridazine

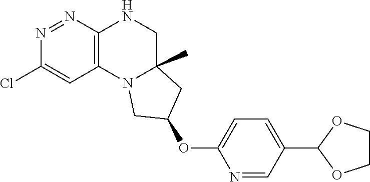 [SMILES: C[C@]12CNc3nnc(Cl)cc3N1C[C@H](Oc1ccc(C3OCCO3)cn1)C2]

To a solution of (6aS,8R)-2-chloro-6a-methyl-5,6,6a,7,8,9-hexahydropyrrolo[1′,2′:4,5]pyrazino[2,3-c]pyridazin-8-ol (300 mg, 1.25 mmol) in THE (5.0 mL) and DMF (0.5 mL) was added 5-(1,3-dioxolan-2-yl)-2-fluoropyridine (274 mg, 1.62 mmol) and sodium hydride (99.7 mg, 2.49 mmol) at 0° C. The reaction was warmed to room temperature and stirred for 1 h. The reaction was quenched with ice cold water. The mixture was diluted with 3:1 CHCl₃/IPA solution (10 mL), and the organic layer was removed, washed with water and brine, dried over Na₂SO₄, and concentrated. The crude product was purified by silica gel chromatography (0-10% MeOH/DCM) to afford the title compound (320 mg, 0.82 mmol, 66% yield). LCMS calc. for C₁₈H₂₁ClN₅O₃[M+H]⁺m/z=390.1; Found 390.1.

### Step 2: (6aS,8R)-8-((5-(1,3-Dioxolan-2-yl)pyridin-2-yl)oxy)-2-(2-(methoxymethoxy)phenyl)-6a-methyl-5,6,6a,7,8,9-hexahydropyrrolo[1′,2′:4,5]pyrazino[2,3-c]pyridazine

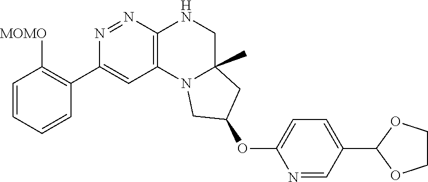

A solution of(6aS,8R)-8-((5-(1,3-dioxolan-2-yl)pyridin-2-yl)oxy)-2-chloro-6a-methyl-5,6,6a,7,8,9-hexahydropyrrolo[1′,2′:4,5]pyrazino[2,3-c]pyridazine (300 mg, 0.77 mmol), cataCXium A Pd G3 (CAS: 1651823-59-4) (56 mg, 0.077 mmol), K₃PO₄ (980 mg, 4.62 mmol) and (2-(methoxymethoxy)phenyl)boronic acid (280 mg, 1.54 mmol) in 1,4-dioxane (4.0 mL) and water (1.0 mL) was sparged with nitrogen for 15 min. The mixture was heated at 90° C. for 1 h. The reaction mixture was cooled to room temperature and diluted with 3:1 CHCl₃/IPA solution (10 mL). The organic layer was removed, washed with water and brine, dried over Na₂SO₄, and concentrated. The crude product was purified by silica gel chromatography (0-10% MeOH/DCM) to afford the title compound (310 mg, 0.63 mmol, 82% yield). LCMS calc. for C₂₆H₃₀N₅O₅ [M+H]⁺m/z=492.2; Found 492.1.

### Step 3: 6-(((6aS,8R)-2-(2-(Methoxymethoxy)phenyl)-6a-methyl-5,6,6a,7,8,9-hexahydropyrrolo[1′,2′:4,5]pyrazino[2,3-c]pyridazin-8-yl)oxy)nicotinaldehyde

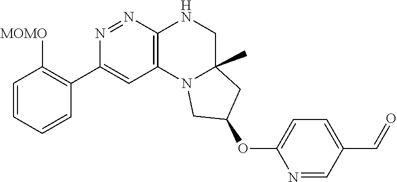

To a solution of (6aS,8R)-8-((5-(1,3-dioxolan-2-yl)pyridin-2-yl)oxy)-2-(2-(methoxymethoxy)phenyl)-6a-methyl-5,6,6a,7,8,9-hexahydropyrrolo[1′,2′:4,5]pyrazino[2,3-c]pyridazine (140 mg, 0.28 mmol) in acetone (1.0 mL) and water (1.0 mL) was added pyridinium p-toluenesulfonate (501 mg, 1.99 mmol). The reaction mixture was stirred at 70° C. for 2 h. The reaction mixture was cooled to room temperature and diluted with 3:1 CHCl₃/IPA solution (10 mL). The organic layer was removed, washed with water and brine, dried over Na₂SO₄, and concentrated. The crude material, which contained the title compound, was carried forward without further purification. LCMS calc. for C₂₄H₂₆N₅O₄ [M+H]⁺m/z=448.2; Found 448.1.

### Step 4: (S)-3-(6-(4-((6-(((6aS,8R)-2-(2-(Methoxymethoxy)phenyl)-6a-methyl-5,6,6a,7,8,9-hexahydropyrrolo[1′,2′:4,5]pyrazino[2,3-c]pyridazin-8-yl)oxy)pyridin-3-yl)methyl)piperazin-1-yl)-1-oxoisoindolin-2-yl)piperidine-2,6-dione

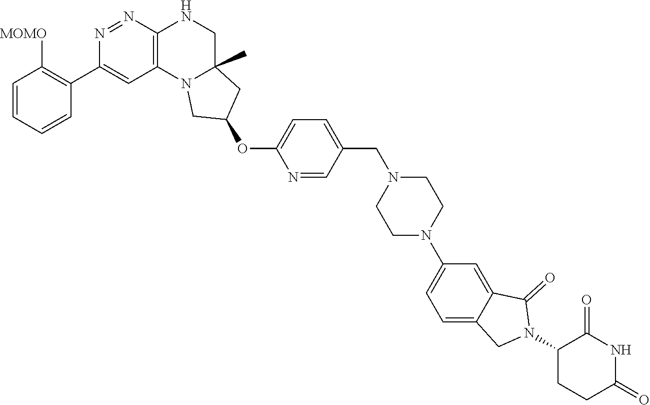

To a solution of 6-(((6aS,8R)-2-(2-(methoxymethoxy)phenyl)-6a-methyl-5,6,6a,7,8,9-hexahydropyrrolo[1′,2′:4,5]pyrazino[2,3-c]pyridazin-8-yl)oxy)nicotinaldehyde (100 mg, 0.22 mmol) and (S)-3-(1-oxo-6-(piperazin-1-yl)isoindolin-2-yl)piperidine-2,6-dione (110 mg, 0.34 mmol) in MeCN (6 mL) was added N,N-diisopropylethylamine (0.27 mL, 1.6 mmol). The reaction mixture was stirred for 2 h. Sodium triacetoxyborohydride (140 mg, 0.67 mmol) was added to the reaction mixture. The resulting mixture was stirred overnight. The reaction mixture was diluted with water and purified by prep-HPLC on C18 column (14-34% MeCN/0.2% TFA (aq.)) to give the title compound as a TFA salt (130 mg, 0.15 mmol, 67% yield). LCMS calc. for C₄₁H₄₆N₉O₆ [M+H]⁺m/z=760.4; Found 760.2.

### Step 5: 4—((S)-2-((S)-2-(6-(2,5-Dioxo-2,5-dihydro-1H-pyrrol-1-yl)hexanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-(methoxymethoxy)phenyl)-6a-methyl-6a,7,8,9-tetrahydropyrrolo[1′,2′:4,5]pyrazino[2,3-c]pyridazine-5(6H)-carboxylate

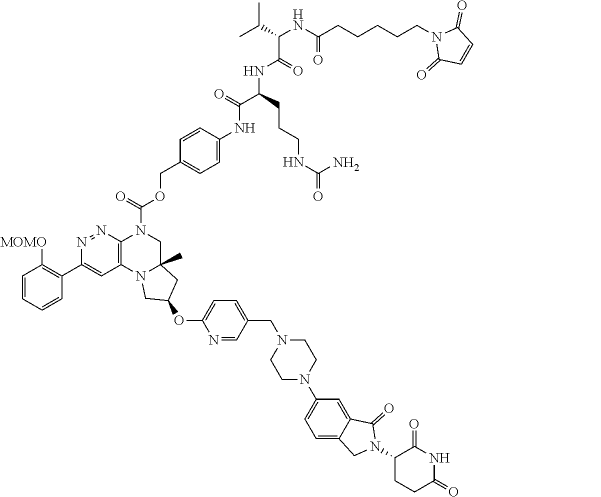

To a solution of (S)-3-(6-(4-((6-(((6aS,8R)-2-(2-(methoxymethoxy)phenyl)-6a-methyl-5,6,6a,7,8,9-hexahydropyrrolo[1′,2′:4,5]pyrazino[2,3-c]pyridazin-8-yl)oxy)pyridin-3-yl)methyl)piperazin-1-yl)-1-oxoisoindolin-2-yl)piperidine-2,6-dione (30 mg, 0.034 mmol) in DMF (1.0 mL) was added MC-Val-Cit-PAB-PNP (28 mg, 0.037 mmol) and 2,6-lutidine (80 uL, 0.69 mmol). The reaction mixture was stirred at 50° C. for 60 h. The reaction was cooled to room temperature, diluted with MeCN, and purified by prep-HPLC on C18 column (19-39% MeCN/0.2% TFA (aq.)) to give the title compound as a TFA salt (8.0 mg, 0.0054 mmol, 16% yield). LCMS calc. for C₇₀H₈₅N₁₅O₁₄ [M+2H]2+m/z=679.8; Found 680.2.

### Step 6: 4—((S)-2-((S)-2-(6-(2,5-Dioxo-2,5-dihydro-1H-pyrrol-1-yl)hexanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-hydroxyphenyl)-6a-methyl-6a,7,8,9-tetrahydropyrrolo[1′,2′:4,5]pyrazino[2,3-c]pyridazine-5(6H)-carboxylate

A solution of 4-((S)-2-((S)-2-(6-(2,5-dioxo-2,5-dihydro-1H-pyrrol-1-yl)hexanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-(methoxymethoxy)phenyl)-6a-methyl-6a,7,8,9-tetrahydropyrrolo[1′,2′:4,5]pyrazino[2,3-c]pyridazine-5(6H)-carboxylate (8.0 mg, 0.0054 mmol) in 1,4-dioxane (2.0 mL) and water (2.0 mL) was added HCl (1.0 mL, 4 N in 1,4-dioxane/water). The reaction mixture was stirred for 90 min. The mixture was diluted with MeCN and purified by prep-HPLC on C18 column (18-38% MeCN/0.2% TFA (aq.)) to give the title compound as a TFA salt (2.3 mg, 0.0016 mmol, 30% yield). LCMS calc. for C₆₈H₈₁N₁₅O₁₃ [M+2H]²⁺ m/z=657.8; Found 657.5.

### Example 7. Synthesis of Compound 2.13—4-((S)-2-((S)-2-(3-(2-(2,5-Dioxo-2,5-dihydro-1H-pyrrol-1-yl)ethoxy)propanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-hydroxyphenyl)-6a-methyl-6a,7,8,9-tetrahydropyrrolo[1′,2′:4,5]pyrazino[2,3-c]pyridazine-5(6H)-carboxylate

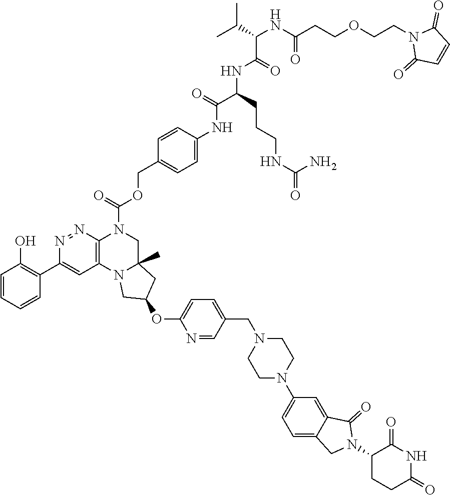 [SMILES: CC(C)[C@H](NC(=O)CCOCCN1C(=O)C=CC1=O)C(=O)N[C@@H](CCCNC(N)=O)C(=O)Nc1ccc(COC(=O)N2C[C@]3(C)C[C@@H](Oc4ccc(CN5CCN(c6ccc7c(c6)C(=O)N([C@H]6CCC(=O)NC6=O)C7)CC5)cn4)CN3c3cc(-c4ccccc4O)nnc32)cc1]

### Step 1: 4—((S)-2-((S)-2-((tert-Butoxycarbonyl)amino)-3-methylbutanamido)-5-ureidopentanamido)benzyl (6aS,8R)-8-((5-formylpyridin-2-yl)oxy)-2-(2-(methoxymethoxy)phenyl)-6a-methyl-6a,7,8,9-tetrahydropyrrolo[1′,2′:4,5]pyrazino[2,3-c]pyridazine-5(6H)-carboxylate

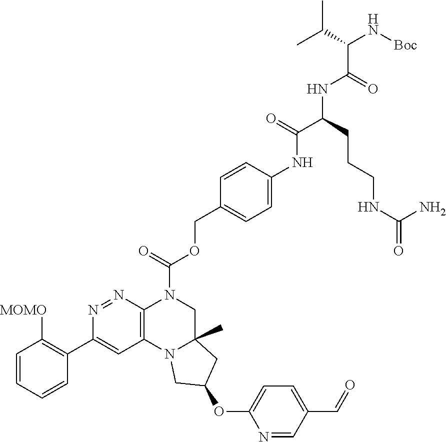

To the solution of (6aS,8R)-8-((5-(1,3-dioxolan-2-yl)pyridin-2-yl)oxy)-2-(2-(methoxymethoxy)phenyl)-6a-methyl-5,6,6a,7,8,9-hexahydropyrrolo[1′,2′:4,5]pyrazino[2,3-c]pyridazine (30 mg, 0.061 mmol) and tert-butyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (39 mg, 0.061 mmol) in DMF (1 mL) was added 2,6-dimethylpyridine (0.14 mL, 1.2 mmol). The mixture was stirred at 50° C. overnight. The reaction mixture was diluted with MeCN and purified by prep-HPLC on C18 column (31-51% MeCN/0.2% TFA (aq.)) to give the title compound as a TFA salt (55 mg, 0.047 mmol, 77% yield), a colorless solid. LCMS calc. for C₄₈H₆₁N₁₀O₁₁ [M+H]⁺m/z=953.4; Found 953.5.

### Step 2: 4—((S)-2-((S)-2-((tert-Butoxycarbonyl)amino)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-(methoxymethoxy)phenyl)-6a-methyl-6a,7,8,9-tetrahydropyrrolo[1′,2′:4,5]pyrazino[2,3-c]pyridazine-5(6H)-carboxylate

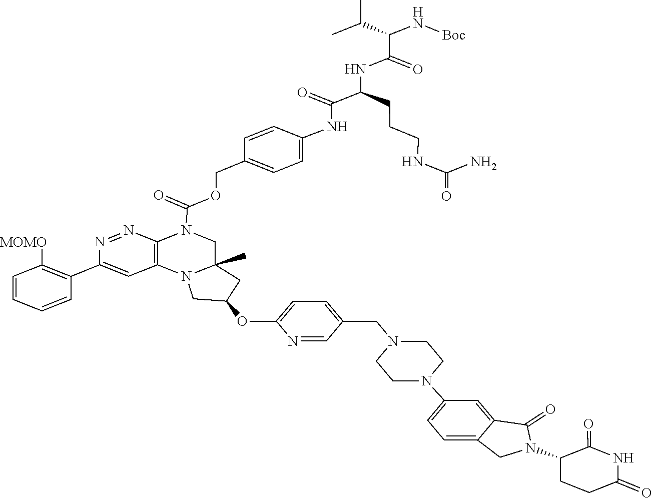

The title compound was synthesized by procedures analogous to those outlined in Example 6, Step 4. LCMS calc. for C₆₅H₈₂N₁₄O₁₃ [M+2H]²⁺ m/z=633.3; Found 633.2

### Step 3: 4—((S)-2-((S)-2-Amino-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-hydroxyphenyl)-6a-methyl-6a,7,8,9-tetrahydropyrrolo[1′,2′:4,5]pyrazino[2,3-c]pyridazine-5(6H)-carboxylate

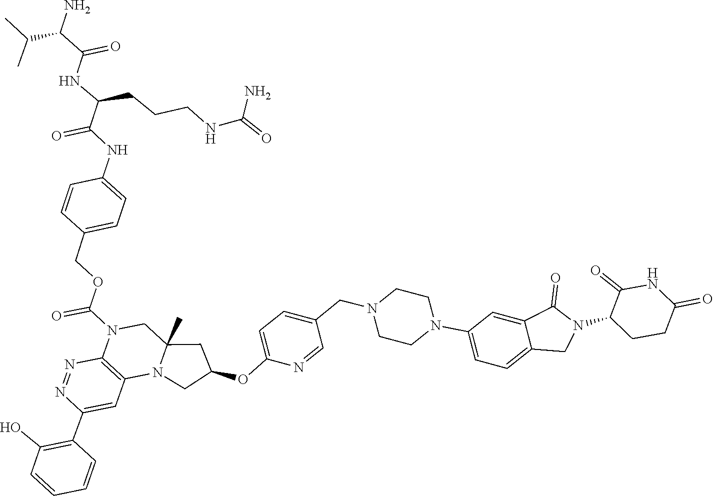 [SMILES: CC(C)[C@H](N)C(=O)N[C@@H](CCCNC(N)=O)C(=O)Nc1ccc(COC(=O)N2C[C@]3(C)C[C@@H](Oc4ccc(CN5CCN(c6ccc7c(c6)C(=O)N([C@H]6CCC(=O)NC6=O)C7)CC5)cn4)CN3c3cc(-c4ccccc4O)nnc32)cc1]

To 4-((S)-2-((S)-2-((tert-butoxycarbonyl)amino)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-(methoxymethoxy)phenyl)-6a-methyl-6a,7,8,9-tetrahydropyrrolo[1′,2′:4,5]pyrazino[2,3-c]pyridazine-5(6H)-carboxylate (10 mg, 0.0079 mmol) was added IPA (0.5 mL) and 6 N HCl (aq.) (0.5 mL). The mixture was stirred for 2 h. The mixture was diluted with MeCN and purified by prep-HPLC on C18 column (21-41% MeCN/0.2% TFA (aq.)) to give the title compound as a TFA salt (8 mg, 0.006 mmol, 80% yield). LCMS calc. for C₅₈H₇₀N₁₄O₁₀ [M+2H]2+m/z=561.3; Found 561.2

### Step 4: 4—((S)-2-((S)-2-(3-(2-(2,5-Dioxo-2,5-dihydro-1H-pyrrol-1-yl)ethoxy)propanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)pyridin-2-yl)oxy)-2-(2-hydroxyphenyl)-6a-methyl-6a,7,8,9-tetrahydropyrrolo[1′,2′:4,5]pyrazino[2,3-c]pyridazine-5(6H)-carboxylate

The title compound was synthesized by procedures analogous to those outlined in Example 5, Step 2. LCMS calc. for C₆₇H₇₉N₁₅O₁₄ [M+2H]2+m/z=658.8; Found 658.8.

### Example 8. Synthesis of Compound 2.37—4-((S)-5-Benzyl-18-(2,5-dioxo-2,5-dihydro-1H-pyrrol-1-yl)-4,7,10,13-tetraoxo-3,6,9,12-tetraazaoctadecanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

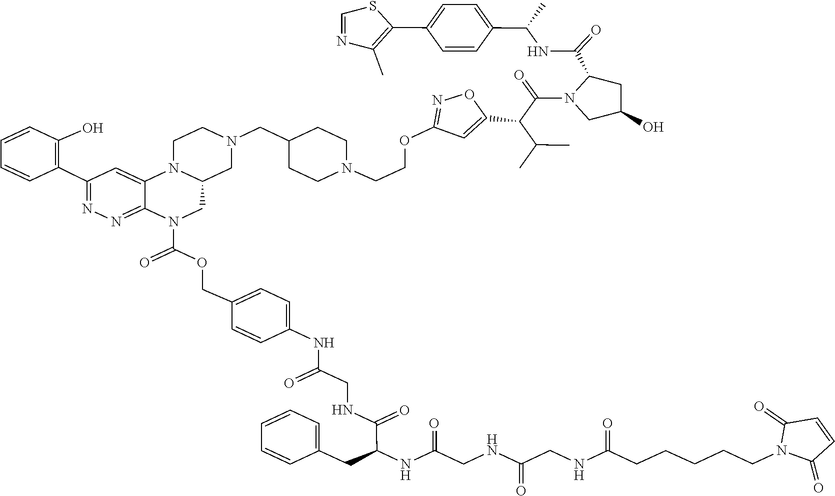 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)CNC(=O)[C@H](Cc7ccccc7)NC(=O)CNC(=O)CNC(=O)CCCCCN7C(=O)C=CC7=O)cc6)C[C@H]5C4)CC3)no2)C(C)C)cc1]

### Step 1: (S)-4-(5-Benzyl-18-(2,5-dioxo-2,5-dihydro-1H-pyrrol-1-yl)-4,7,10,13-tetraoxo-3,6,9,12-tetraazaoctadecanamido)benzyl (4-nitrophenyl) carbonate

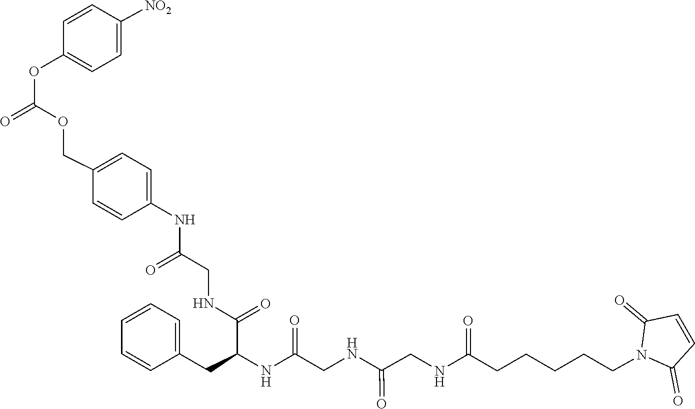 [SMILES: O=C(CCCCCN1C(=O)C=CC1=O)NCC(=O)NCC(=O)N[C@@H](Cc1ccccc1)C(=O)NCC(=O)Nc1ccc(COC(=O)Oc2ccc([N+](=O)[O-])cc2)cc1]

The title compound was synthesized by procedures analogous to those outlined in Example 4, Step 4 from Mc-Gly-Gly-Phe-Gly-PAB-OH (CAS #2632342-05-1). LCMS calc. for C₃₉H₄₂N₇O₁₂ [M+H]⁺: m/z=800.3; Found 800.8.

### Step 2: 4—((S)-5-Benzyl-18-(2,5-dioxo-2,5-dihydro-1H-pyrrol-1-yl)-4,7,10,13-tetraoxo-3,6,9,12-tetraazaoctadecanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

The title compound was synthesized by procedures analogous to those outlined in Example 3, Steps 5-6. LCMS calc. for C₈₁H₉₈N₁₆O₁₅S [M+2H]²⁺ m/z=783.4; Found 783.5.

### Example 9. Synthesis of Compound 2.50—4-((S)-2-((S)-2-(2-(2,5-Dioxo-2,5-dihydro-1H-pyrrol-1-yl)acetamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

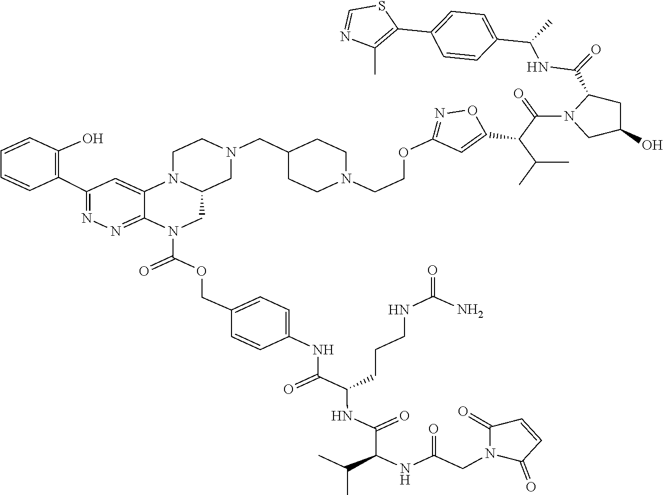 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CN7C(=O)C=CC7=O)C(C)C)cc6)C[C@H]5C4)CC3)no2)C(C)C)cc1]

To a solution of 4-((S)-2-((S)-2-amino-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate (19 mg, 0.015 mmol, Example 5, Step 1) in N-methyl-2-pyrrolidone (0.40 mL) was added N,N-diisopropylethylamine (0.025 mL, 0.14 mmol). The reaction mixture was stirred for 5 min, and then (2,5-dioxopyrrolidin-1-yl) 2-(2,5-dioxopyrrol-1-yl)acetate (5.5 mg, 0.022 mmol) was added. The resulting mixture was stirred for 4 h. The reaction mixture was diluted with MeCN and purified by prep-HPLC on C18 column (5-95% MeCN/0.1% TFA (aq.)) to give the title compound as a TFA salt (7.0 mg, 0.0042 mmol, 28% yield) as a white solid. LCMS calc. for C₇₃H₉₁N₁₆O₁₄S [M+H]m/z=1447.7; Found 1447.6.

### Example 10. Synthesis of Compound 2.23—4-((S)-2-((S)-2-(6-(2-Iodoacetamido)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

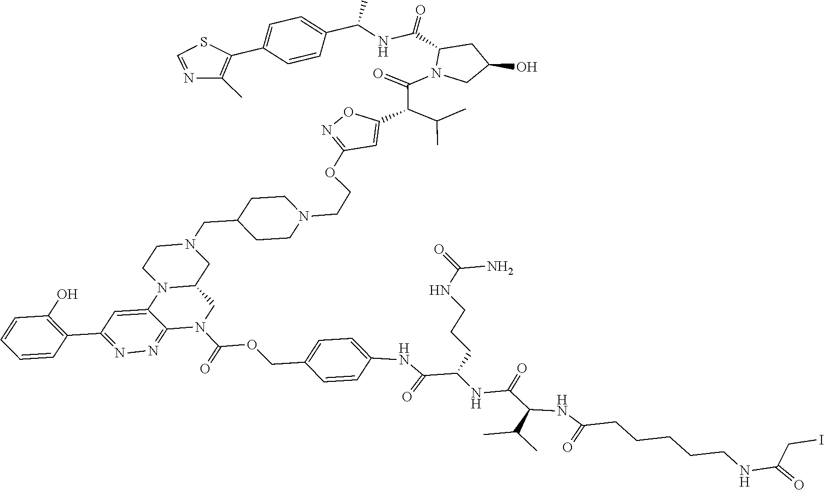 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCNC(=O)CI)C(C)C)cc6)C[C@H]5C4)CC3)no2)C(C)C)cc1]

### Step 1: 4-((13S,16S)-13-Isopropyl-2,2-dimethyl-4,11,14-trioxo-16-(3-ureidopropyl)-3-oxa-5,12,15-triazaheptadecan-17-amido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

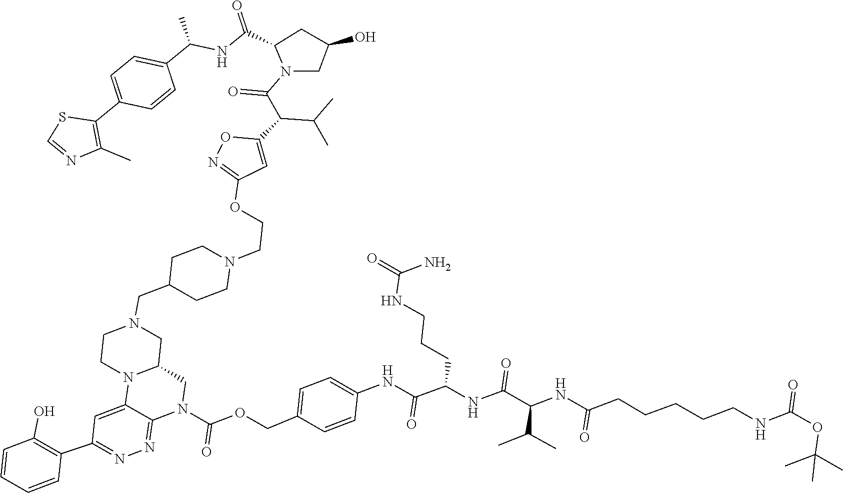 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCNC(=O)OC(C)(C)C)C(C)C)cc6)C[C@H]5C4)CC3)no2)C(C)C)cc1]

To a solution of 4-((S)-2-((S)-2-amino-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate (19 mg, 0.015 mmol, from Example 3, Step 4) in DMF (2 mL) was added triethylamine (4.1 μL, 0.029 mmol) and (2,5-dioxopyrrolidin-1-yl) 6-[(2-methylpropan-2-yl)oxycarbonylamino]hexanoate (9.6 mg, 0.029 mmol). The reaction mixture was stirred for 20 min. The crude material was purified by prep-HPLC on a C18 column (10-70% MeCN/0.1% TFA (aq.)) to afford a TFA salt of the title compound (8.4 mg, 0.0055 mmol, 38% yield) as a colorless solid. LC-MS calc. for C₇₈H₁₀₈N₁₆O₁₄S [M+2H]2+: m/z=762.2; Found: 762.3.

### Step 2. 4—((S)-2-((S)-2-(6-Aminohexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

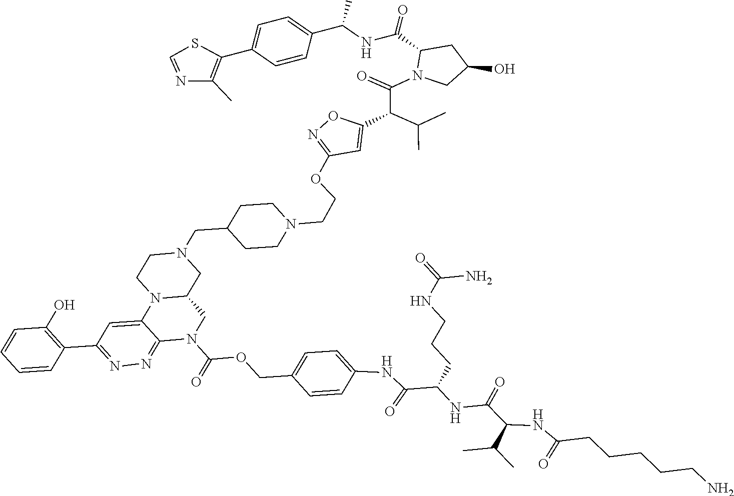 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCN)C(C)C)cc6)C[C@H]5C4)CC3)no2)C(C)C)cc1]

To a solution of 4-((13S,16S)-13-isopropyl-2,2-dimethyl-4,11,14-trioxo-16-(3-ureidopropyl)-3-oxa-5,12,15-triazaheptadecan-17-amido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate (8.4 mg, 0.0055 mmol) in THE (0.50 mL) and IPA (0.50 mL) was added 12 N HCl (aq.) (6.9 μL, 0.083 mmol). The reaction mixture was stirred for 5 min. The mixture was diluted with water and purified by prep-HPLC on a C18 column (10-50% MeCN/0.05% TFA (aq)) to afford the title compound as a TFA salt (4.9 mg, 0.0034 mmol, 62% yield), a colorless solid. LC-MS calc. for C₇₃H₉₉N₁₆O₁₂S [M+H]⁺: m/z=1423.7; Found: 1423.6.

### Step 3. 4—((S)-2-((S)-2-(6-(2-Iodoacetamido)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

To a solution of 4-((S)-2-((S)-2-(6-aminohexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate (5.0 mg, 0.0035 mmol) in DMF (1 mL) was added triethylamine (1.5 μL, 0.011 mmol) and (2,5-dioxopyrrolidin-1-yl) 2-iodoacetate (2.0 mg, 0.0070 mmol). The resulting mixture was stirred for 5 min. The crude material was purified by prep-HPLC on a C18 column (10-60% MeCN/0.05% TFA (aq)) to afford the title compound as a TFA salt (2.1 mg, 0.0013 mmol, 38% yield). LC-MS calc. for C₇₅H₁₀₀IN₁₆O₁₃S [M+H]⁺: m/z=1591.6; Found: 1592.0.

### Example 11. Synthesis of Compound 2.17—4-((S)-2-((S)-2-(6-(3,4-Dibromo-2,5-dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl- I-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

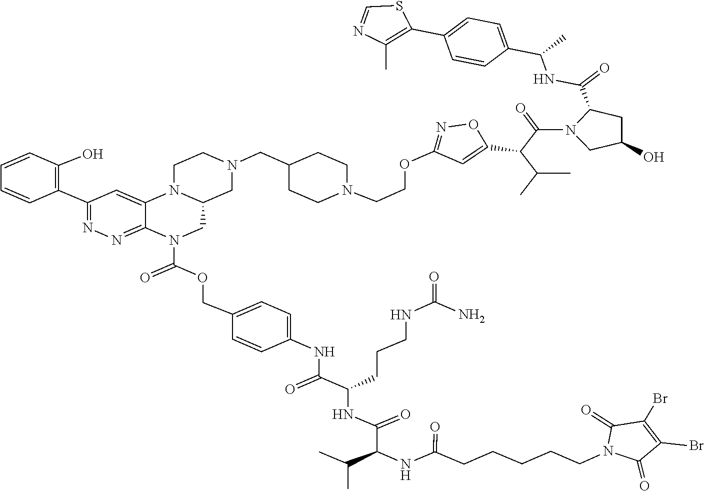 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C(Br)=C(Br)C7=O)C(C)C)cc6)C[C@H]5C4)CC3)no2)C(C)C)cc1]

### Step 1. 6-(2,5-Dioxo-2,5-dihydro-1H-pyrrol-1-yl)hexanoic acid

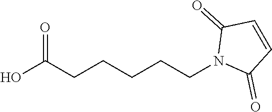 [SMILES: O=C(O)CCCCCN1C(=O)C=CC1=O]

A solution of 6-aminohexanoic acid (1.08 g, 8.22 mmol) and maleic anhydride (806 mg, 8.22 mmol) in acetic acid (10 mL) was refluxed for 6 h. The mixture was cooled to room temperature and used directly in the next step. LC-MS calc. for C₁₀H₁₄NO₄ [M+H]⁺: m/z=212.1; Found: 212.1.

### Step 2. 6-(3,4-Dibromo-2,5-dioxo-2,5-dihydro-1H-pyrrol-1-yl)hexanoic acid

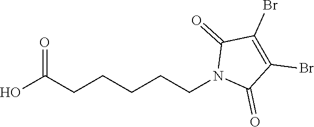 [SMILES: O=C(O)CCCCCN1C(=O)C(Br)=C(Br)C1=O]

To a solution of 6-(2,5-dioxo-2,5-dihydro-1H-pyrrol-1-yl)hexanoic acid (0.58 g, 2.7 mmol) in acetic acid (3 mL) was added bromine (0.31 mL, 6.0 mmol) and sodium acetate (495 mg, 6.03 mmol) at 0° C. The mixture was stirred at 135° C. for 4 h. The reaction mixture was cooled to room temperature and diluted with sat. sodium thiosulfate (aq.) (10 mL) and EtOAc (10 mL). The organic phase was separated, and the aqueous phase was extracted with EtOAc (3×10 mL). The combined organic phases were dried over Na₂SO₄, concentrated, and purified by silica gel chromatography (10-60% EtOAc/heptane) to afford the title compound (150 mg, 0.406 mmol, 14.8% yield) as a colorless solid. ¹H NMR (300 MHz, CDCl₃) δ 3.65 (t, J=7.2 Hz, 2H), 2.40 (t, J=7.4 Hz, 2H), 1.69 (h, J=7.3 Hz, 4H), 1.45-1.33 (m, 2H).

### Step 3. 4—((S)-2-((S)-2-((((9H-Fluoren-9-yl)methoxy)carbonyl)amino)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate

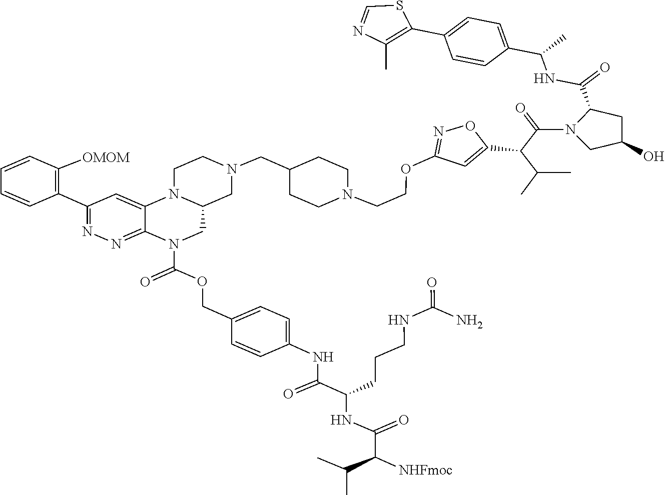

To a solution of (2S,4R)-4-((tert-butyldimethylsilyl)oxy)-1-((R)-2-(3-(2-(4-(((S)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (680 mg, 0.639 mmol, from Example 3, Step 4) and (9H-fluoren-9-yl)methyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate (735 mg, 0.959 mmol) in DMF (10 mL) was added 2,6-lutidine (1.34 mL, 11.5 mmol). The reaction mixture was stirred for 24 h. The mixture was poured into water (50 mL), and the resulting solid was collected by filtration and further purified by prep-HPLC on a C18 column (10-60% MeCN/0.5% TFA (aq)) to afford the title compound as a TFA salt (550 mg, 0.325 mmol, 50.861% yield), a colorless solid. LC-MS calc. for C₈₄H₁₀₂N₁₅O₁₄S [M+H]⁺: m/z=1576.7; Found: 1576.9.

### Step 4. 4—((S)-2-((S)-2-Amino-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate

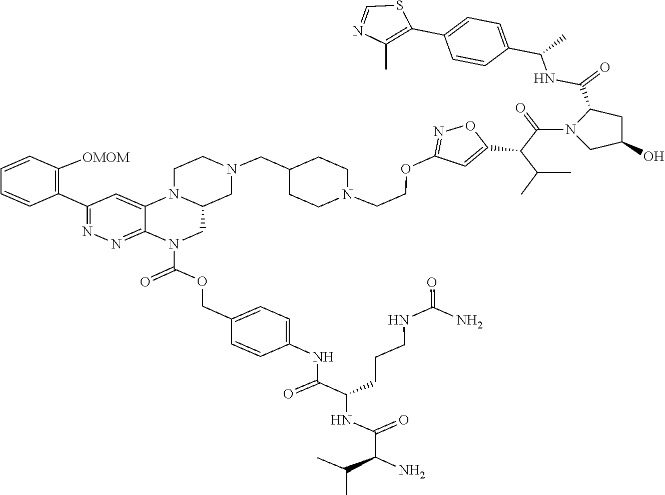

To a solution of 4-((S)-2-((S)-2-((((9H-fluoren-9-yl)methoxy)carbonyl)amino)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate (174 mg, 0.110 mmol) in DMF (2 mL) was added 1,8-diazabicyclo[5.4.0]undec-7-ene (82.4 μL, 0.552 mmol). The mixture was stirred for 16 h, washed with water (10 mL), dried over Na₂SO₄, concentrated, and purified by silica gel chromatography (0-10% MeOH/DCM). The material was dissolved in DCM (20 mL), washed with sat. aq. NaHCO₃ (2×10 mL), dried over Na₂SO₄, and concentrated to afford the title compound (101 mg, 0.0745 mmol, 67.5% yield) as a colorless solid. LC-MS calc. for C₆₉H₉₂N₁₅O₁₂S [M+H]⁺m/z=1354.7; Found 1354.6.

### Step 5. 4—((S)-2-((S)-2-(6-(3,4-Dibromo-2,5-dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate

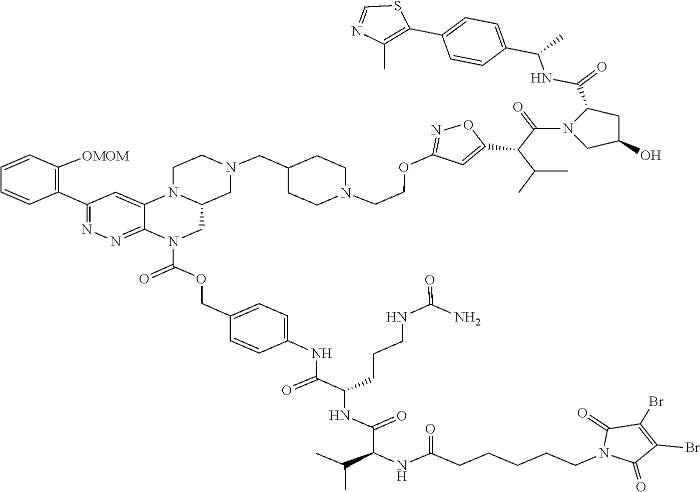

A solution of 6-(3,4-dibromo-2,5-dioxo-2,5-dihydro-1H-pyrrol-1-yl)hexanoic acid (8.0 mg, 0.022 mmol) and N-ethoxycarbonyl-2-ethoxy-1,2-dihydroquinoline (5.4 mg, 0.022 mmol) in MeCN (1 mL) was stirred in the dark for 1 h. To the mixture was added a solution of 4-((S)-2-((S)-2-amino-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate (15 mg, 0.011 mmol) in DMF (0.5 mL). The reaction mixture was stirred for 1 h. The mixture was filtered and purified by prep-HPLC on a C18 column (5-50% MeCN/0.05% TFA (aq.)) to afford the title compound as a TFA salt (4.0 mg, 0.0024 mmol, 22% yield), a colorless solid. LC-MS calc. for C₇₉H₁₀₁Br₂N₁₆O₁₅S [M+H]⁺m/z=1705.6; Found 1705.5.

### Step 6. 4—((S)-2-((S)-2-(6-(3,4-Dibromo-2,5-dioxo-2,5-dihydro-1H-pyrrol-1-yl)hexanamido)-3-methylbutanamido)-5-ureidopentanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

The title compound was synthesized according to procedures analogous to Example 3, Step 6. LC-MS calc. for C₇₇H₉₇Br₂N₁₆O₁₄S [M+H]⁺m/z=1661.5; Found 1661.5.

### Example 12. Synthesis of Compound 2.51—4-((R)-2-(4-Aminobutyl)-35-(4-(((1r,4r)-4-((2,5-dioxo-2,5-dihydro-1H-pyrrol-1-yl)methyl)cyclohexane-1-carboxamido)methyl)-1H-1,2,3-triazol-1-yl)-4,8-dioxo-6,12,15,18,21,24,27,30,33-nonaoxa-3,9-diazapentatriacontanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

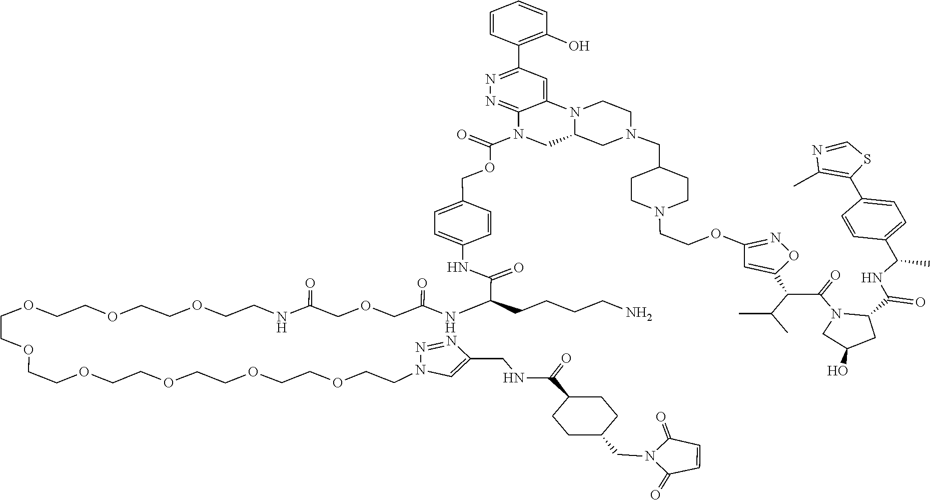 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(C(=O)OCc6ccc(NC(=O)[C@@H](CCCCN)NC(=O)COCC(=O)NCCOCCOCCOCCOCCOCCOCCOCCOCCn7cc(CNC(=O)[C@H]8CC[C@H](CN9C(=O)C=CC9=O)CC8)nn7)cc6)C[C@H]5C4)CC3)no2)C(C)C)cc1]

### Step 1. (1r,4r)-4-((2,5-Dioxo-2,5-dihydro-1H-pyrrol-1-yl)methyl)-N-(prop-2-yn-1-yl)cyclohexane-1-carboxamide

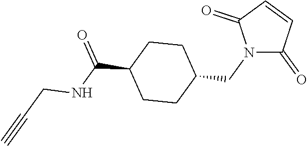 [SMILES: C#CCNC(=O)[C@H]1CC[C@H](CN2C(=O)C=CC2=O)CC1]

To a solution of (1r,4r)-4-((2,5-dioxo-2,5-dihydro-1H-pyrrol-1-yl)methyl)cyclohexane-1-carboxylic (200 mg, 0.60 mmol) in THE (3.0 mL) and water (3.0 mL) was added NaHCO₃ (150 mg, 1.8 mmol) and prop-2-yn-1-amine (40 μL, 0.63 mmol). The reaction mixture was stirred for 1 h then concentrated. The crude residue was purified by silica gel chromatography (0-20% MeOH/DCM) to give the title compound (140 mg, 0.49 mmol, 83% yield) as a white solid. LC-MS calc. for C₁₅H₁₉N₂O₃ [M+H]⁺: m/z=275.1; Found: 275.2.

### Step 2. (9H-Fluoren-9-yl)methyl (R)-(6-((diphenyl(p-tolyl)methyl)amino)-1-((4-(hydroxymethyl)phenyl)amino)-1-oxohexan-2-yl)carbamate

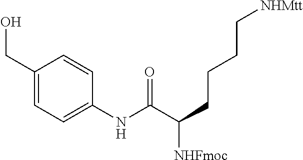

To a solution of N²-(((9H-fluoren-9-yl)methoxy)carbonyl)-N⁶-(diphenyl(p-tolyl)methyl)-D-lysine (300 mg, 0.48 mmol) in THE (2.5 mL) was added (4-aminophenyl)methanol (77 mg, 0.62 mmol) and EEDQ (150 mg, 0.62 mmol). The reaction mixture was stirred at room temperature overnight then concentrated. The crude residue was purified by silica gel chromatography (0%-80% EtOAc/heptane) to give the title compound (320 mg, 0.44 mmol, 91% yield) as a beige solid. LCMS calcd for C₄₈H₄₈N₃O₄ [M+H]⁺: m/z=730.4; Found: 730.3.

### Step 3. (R)-2-Amino-6-((diphenyl(p-tolyl)methyl)amino)-N-(4-(hydroxymethyl)phenyl)hexanamide

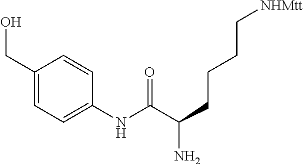

To a solution of (9H-fluoren-9-yl)methyl (R)-(6-((diphenyl(p-tolyl)methyl)amino)-1-((4-(hydroxymethyl)phenyl)amino)-1-oxohexan-2-yl)carbamate (320 mg, 0.44 mmol) in THE (2.9 mL) was added piperidine (86 μL, 0.87 mmol). The reaction mixture was stirred for 1 h then concentrated. The crude residue was purified by silica gel chromatography (0-40% MeOH/MTBE) to give the title compound (220 mg, 0.44 mmol, >99% yield). LCMS calcd for C₃₃H₃₈N₃O₂ [M+H]⁺: m/z=508.3; Found: 508.4.

### Step 4. (R)-2-(32-Azido-5-oxo-3,9,12,15,18,21,24,27,30-nonaoxa-6-azadotriacontanamido)-6-((diphenyl(p-tolyl)methyl)amino)-N-(4-(hydroxymethyl)phenyl)hexanamide

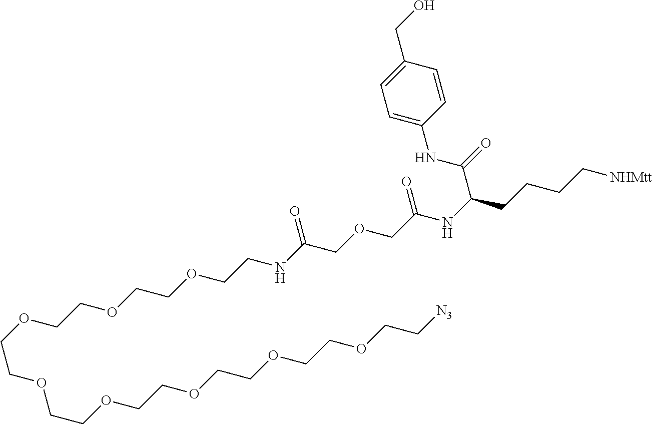

To a solution of (R)-2-amino-6-((diphenyl(p-tolyl)methyl)amino)-N-(4-(hydroxymethyl)phenyl)hexanamide (100 mg, 0.20 mmol) in DCM (2.0 mL) was added 32-azido-5-oxo-3,9,12,15,18,21,24,27,30-nonaoxa-6-azadotriacontanoic acid (120 mg, 0.24 mmol) and EEDQ (59 mg, 0.24 mmol). The reaction mixture was stirred overnight then concentrated. The crude residue was purified by silica gel chromatography (0-40% MeOH/EtOAc) to give the title compound (160 mg, 0.16 mmol, 79% yield) as a yellow oil. LCMS calcd for C₅₅H₇₈N₇O₁₃ [M+H]⁺: m/z=1044.6; Found: 1044.5.

### Step 5. (1r,4r)-4-((2,5-Dioxo-2,5-dihydro-1H-pyrrol-1-yl)methyl)-N-((1-((R)-7-((4-(hydroxymethyl)phenyl)carbamoyl)-9,13-dioxo-1,1-diphenyl-1-(p-tolyl)-11,17,20,23,26,29,32,35,38-nonaoxa-2,8,14-triazatetracontan-40-yl)-1H-1,2,3-triazol-4-yl)methyl)cyclohexane-1-carboxamide

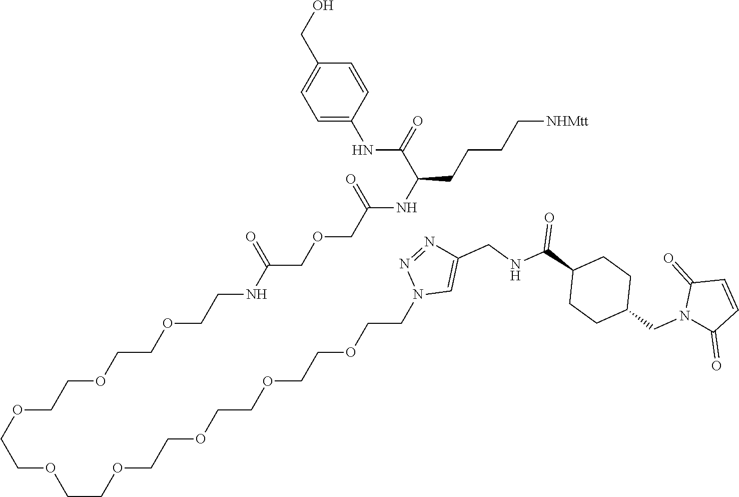

To a solution of (R)-2-(32-azido-5-oxo-3,9,12,15,18,21,24,27,30-nonaoxa-6-azadotriacontanamido)-6-((diphenyl(p-tolyl)methyl)amino)-N-(4-(hydroxymethyl)phenyl)hexanamide (190 mg, 0.18 mmol) and (1r,4r)-4-((2,5-dioxo-2,5-dihydro-1H-pyrrol-1-yl)methyl)-N-(prop-2-yn-1-yl)cyclohexane-1-carboxamide (70 mg, 0.26 mmol, Step 1) in DCM (1.5 mL) was added a solution of triphenylphosphine (7.2 mg, 27 μmol), copper(I) bromide (2.9 mg, 20 μmol), and N,N-diisopropylethylamine (0.48 mL, 2.7 mmol) in DCM (1.5 mL). The reaction mixture was stirred overnight and then concentrated. The crude residue was purified by prep-HPLC on a C18 column (15-50% MeCN/0.1% TFA (aq.)) and the desired fractions were adjusted to pH -8. The resulting solution was concentrated, and the remaining aqueous solution was extracted with 3:1 CHCl₃/IPA (50 mL×3). The combined organic layers were washed with 0.1 N EDTA (aq.), water, dried over sodium sulfate, filtered, and concentrated to afford the title compound (110 mg, 0.085 mmol, 47% yield) as a yellow solid. LCMS calcd for C₇₀H₉₆N₉O₁₆ [M+H]⁺: m/z=1318.7; Found: 1318.9.

### Step 6. 4—((R)-35-(4-(((1r,4r)-4-((2,5-Dioxo-2,5-dihydro-1H-pyrrol-1-yl)methyl)cyclohexane-1-carboxamido)methyl)-1H-1,2,3-triazol-1-yl)-2-(4-((diphenyl(p-tolyl)methyl)amino)butyl)-4,8-dioxo-6,12,15,18,21,24,27,30,33-nonaoxa-3,9-diazapentatriacontanamido)benzyl (4-nitrophenyl) carbonate

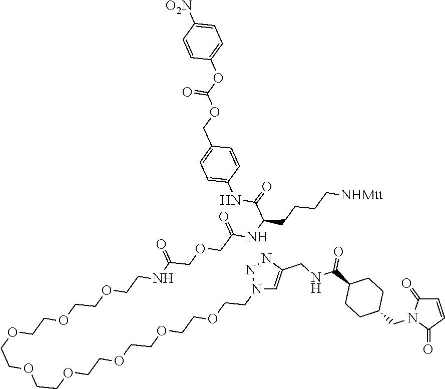

The title compound was synthesized by procedures analogous to those outlined in Example 4, Step 4. LCMS calc. for C₇₇H₁₀₀N₁₀O₂₀ [M+2H]²⁺ m/z=1483.7; Found 1483.8.

### Step 7. 4-((2R)-35-(4-((4-((2,5-Dioxo-2,5-dihydro-1H-pyrrol-1-yl)methyl)cyclohexane-1-carboxamido)methyl)-1H-1,2,3-triazol-1-yl)-2-(4-((diphenyl(p-tolyl)methyl)amino)butyl)-4,8-dioxo-6,12,15,18,21,24,27,30,33-nonaoxa-3,9-diazapentatriacontanamido)benzyl (6aR)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate

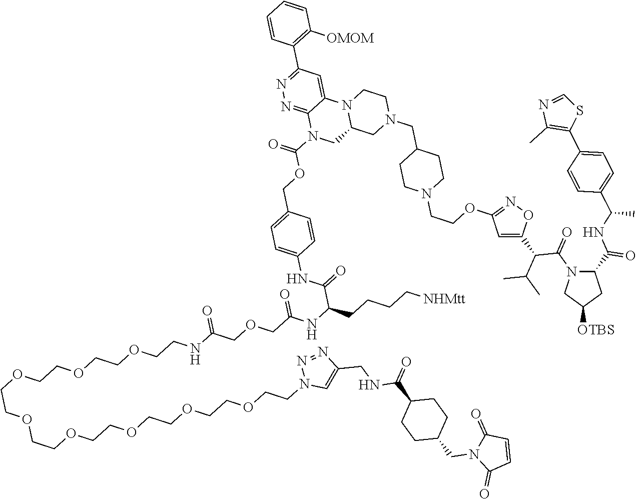

The title compound was synthesized by procedures analogous to those outlined in Example 3, Step 5. LCMS calc. for C₁₂₇H₁₇₃N₁₉O₂₄SSi [M+2H]²⁺ m/z=1204.1; Found 1204.8.

### Step 8. 4—((R)-2-(4-Aminobutyl)-35-(4-(((1r,4r)-4-((2,5-dioxo-2,5-dihydro-1H-pyrrol-1-yl)methyl)cyclohexane-1-carboxamido)methyl)-1H-1,2,3-triazol-1-yl)-4,8-dioxo-6,12,15,18,21,24,27,30,33-nonaoxa-3,9-diazapentatriacontanamido)benzyl (R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate

To a solution of 4-((2R)-35-(4-((4-((2,5-dioxo-2,5-dihydro-1H-pyrrol-1-yl)methyl)cyclohexane-1-carboxamido)methyl)-1H-1,2,3-triazol-1-yl)-2-(4-((diphenyl(p-tolyl)methyl)amino)butyl)-4,8-dioxo-6,12,15,18,21,24,27,30,33-nonaoxa-3,9-diazapentatriacontanamido)benzyl (6aR)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazine-5-carboxylate (47 mg, 19 μmol) in DMF (2.0 mL) was added conc. HCl solution (aq.) (2.0 mL). The reaction mixture was stirred for 20 min and then the crude mixture was purified by prep-HPLC on a C18 column (10-50% MeCN/0.05% TFA (aq.)) to afford the TFA salt of the title compound (23 mg, 11 μmol, 60% yield) as a white solid. LCMS calc. for C₉₉H₁₃₉N₁₉O₂₃S [M+2H]2+m/z=997.0; Found 997.0.

### Example 13. Synthesis of Compound 2.57 —((((29S,29'S,32S,32'S)-1,1′-((6-(2-(2-(2-(2,5-Dioxo-2,5-dihydro-1H-pyrrol-1-yl)acetamido)ethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate)

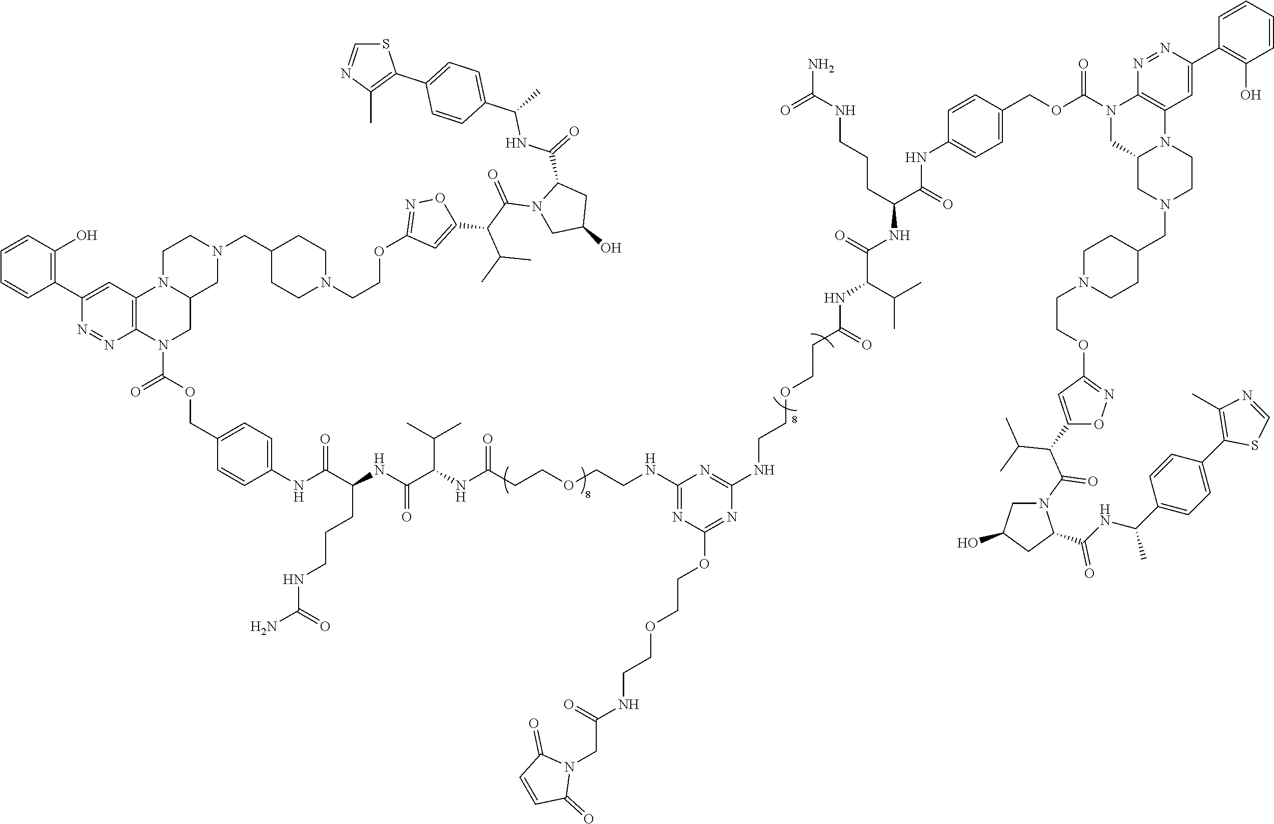

### Step 1. tert-Butyl (2-(2-((4,6-Dichloro-1,3,5-triazin-2-yl)oxy)ethoxy)ethyl)carbamate

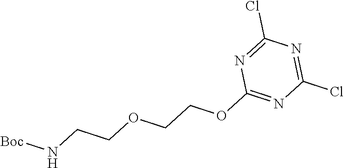 [SMILES: CC(C)(C)OC(=O)NCCOCCOc1nc(Cl)nc(Cl)n1]

To a solution of tert-butyl (2-(2-hydroxyethoxy)ethyl)carbamate (1.0 g, 4.9 mmol) in THE (20 mL) were added cyanuric chloride (1.4 g, 7.3 mmol) and N,N-diisopropylethylamine (1.3 mL, 7.3 mmol) dropwise. The reaction mixture was stirred for 72 h. The mixture was filtered, and the filter cake was washed with THF. The combined filtrate was concentrated, and the crude residue was purified by silica gel chromatography (0-30% EtOAc/heptane) to afford the title compound (1.6 g, 4.7 mmol, 96% yield) as a yellow oil. ¹H-NMR (300 MHz, CDCl₃) δ 4.86 (s, 1H), 4.68-4.57 (m, 2H), 3.87-3.76 (m, 2H), 3.56 (t, J=5.2 Hz, 2H), 3.30 (q, J=5.4 Hz, 2H), 1.43 (s, 9H).

### Step 2. Di-tert-butyl 1,1′-((6-(2-(2-((tert-butoxycarbonyl)amino)ethoxy)ethoxy)-1,3,5-triazine-2,4-diyl)bis(azanediyl))bis(3,6,9,12,15,18,21,24-octaoxaheptacosan-27-oate)

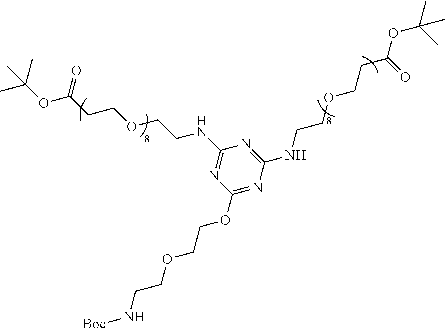

To a solution of tert-butyl (2-(2-((4,6-dichloro-1,3,5-triazin-2-yl)oxy)ethoxy)ethyl)carbamate (150 mg, 0.43 mmol) in 1,4-dioxane (1.0 mL) was added tert-butyl 1-amino-3,6,9,12,15,18,21,24-octaoxaheptacosan-27-oate (420 mg, 0.85 mmol) and N,N-diisopropylethylamine (0.30 mL, 1.7 mmol). After stirring overnight, the mixture was diluted with water (20 mL) and EtOAc (20 mL). The biphasic mixture was separated, and the aqueous layer extracted with EtOAc (20 mL×3). The combined organic layers were washed with brine, dried over sodium sulfate, filtered, and concentrated to afford the title compound (540 mg, 0.42 mmol, >99% yield). LCMS calc. for C₅₈H₁₁N₆O₂₄ [M+H]⁺m/z=1275.8; Found 1275.4.

### Step 3. 1,1′-((6-(2-(2-Aminoethoxy)ethoxy)-1,3,5-triazine-2,4-diyl)bis(azanediyl))bis(3,6,9,12,15,18,21,24-octaoxaheptacosan-27-oic acid)

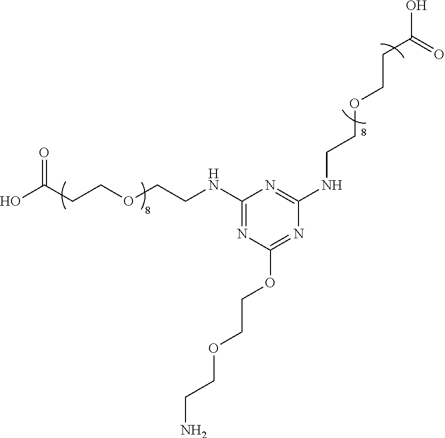

To a solution of crude di-tert-butyl 1,1′-((6-(2-(2-((tert-butoxycarbonyl)amino)ethoxy)ethoxy)-1,3,5-triazine-2,4-diyl)bis(azanediyl))bis(3,6,9,12,15,18,21,24-octaoxaheptacosan-27-oate) (720 mg, 0.57 mmol) in DCM (10 mL) was added TFA (5.0 mL, 65 mmol). After stirring for 3 h, the mixture was concentrated to afford the TFA salt of the title compound (600 mg, 0.57 mmol, 90% yield). LCMS calc. for C₄₅H₈₇N₆O₂₂ [M+H]⁺m/z=1063.6; Found 1063.4.

### Step 4. 1,1′-((6-(2-(2-((tert-Butoxycarbonyl)amino)ethoxy)ethoxy)-1,3,5-triazine-2,4-diyl)bis(azanediyl))bis(3,6,9,12,15,18,21,24-octaoxaheptacosan-27-oic acid)

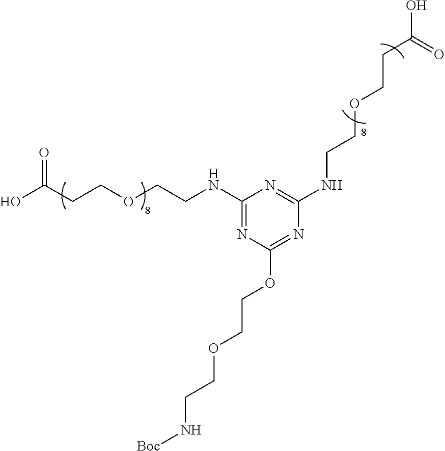

To a solution of 1,1′-((6-(2-(2-aminoethoxy)ethoxy)-1,3,5-triazine-2,4-diyl)bis(azanediyl))bis(3,6,9,12,15,18,21,24-octaoxaheptacosan-27-oic acid) (600 mg, 0.57 mmol) in THE (10 mL) and water (10 mL) was added NaHCO₃ (480 mg, 5.7 mmol) and di-tert butyl dicarbonate (250 mg, 1.1 mmol). After stirring overnight, the mixture was concentrated. The crude residue was diluted with EtOAc (20 mL) and water (10 mL), and then the mixture was adjusted to pH 3-4 with 1 M HCl (aq.). The two layers were separated, and the aqueous layer was extracted with 3:1 CHCl₃/IPA (20 mL×3). The combined organic layers were dried over sodium sulfate, filtered, and concentrated. The crude residue was purified by prep-HPLC on a C18 column (20-100% MeCN/0.1% TFA (aq.)) to afford the TFA salt of the title compound (350 mg, 0.31 mmol, 54% yield) as a light brown solid. LCMS calc. for C₅₀H₉₅N₆O₂₄ [M+H]⁺ m/z=1163.6; Found 1164.2.

### Step 5. Bis(2,5-dioxopyrrolidin-1-yl) 1,1′-((6-(2-(2-((tert-butoxycarbonyl)amino)ethoxy)ethoxy)-1,3,5-triazine-2,4-diyl)bis(azanediyl))bis(3,6,9,12,15,18,21,24-octaoxaheptacosan-27-oate)

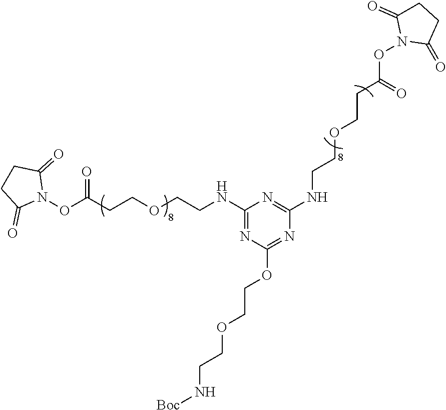

To a solution of 1,1′-((6-(2-(2-((tert-butoxycarbonyl)amino)ethoxy)ethoxy)-1,3,5-triazine-2,4-diyl)bis(azanediyl))bis(3,6,9,12,15,18,21,24-octaoxaheptacosan-27-oic acid) (160 mg, 0.14 mmol) in DCM (4.0 mL) was added 1-ethyl-3-(3-dimethylaminopropyl)carbodiimide (130 mg, 0.68 mmol), 4-dimethylaminopyridine (1.7 mg, 14 μmol), and N-hydroxysuccinimide (64 mg, 0.56 mmol). The reaction mixture was stirred for 3 h and then concentrated. The crude residue was purified by prep-HPLC on a C18 column (15-80% MeCN/0.05% TFA (aq.)) to afford the TFA salt of the title compound (95 mg, 70 μmol, 50% yield) as a brown oil. LCMS calc. for C₅₈H₁₀₁N₈O₂₈ [M+H]⁺m/z=1357.7; Found 1358.5.

### Step 6. 1-((4-(((6S,9S)-1-Amino-6-((4-((((R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carbonyl)oxy)methyl)phenyl)carbamoyl)-9-isopropyl-1,8,11-trioxo-14,17,20,23,26,29,32,35-octaoxa-2,7,10-triazaheptatriacontan-37-yl)amino)-6-(2-(2-((tert-butoxycarbonyl)amino)ethoxy)ethoxy)-1,3,5-triazin-2-yl)amino)-3,6,9,12,15,18,21,24-octaoxaheptacosan-27-oic acid and ((((29S,29'S,32S,32'S)-1,1′-((6-(2-(2-((tert-butoxycarbonyl)amino)ethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate)

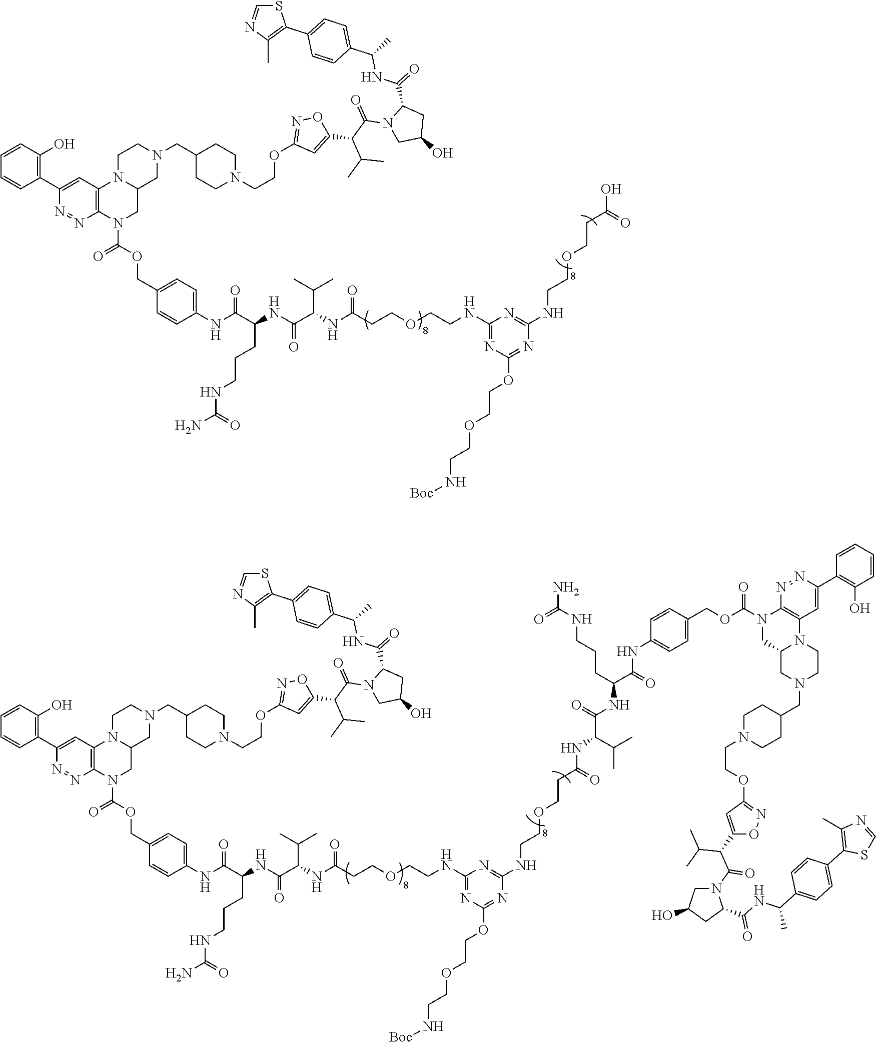

To a solution of 4-((S)-2-((S)-2-amino-3-methylbutanamido)-5-ureidopentanamido)benzyl 8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate (25 mg, 19 μmol, Example 5, Step 1) in DMF (1.0 mL) was added N,N-diisopropylethylamine (16 μL, 94 μmol) and bis(2,5-dioxopyrrolidin-1-yl) 1,1′-((6-(2-(2-((tert-butoxycarbonyl)amino)ethoxy)ethoxy)-1,3,5-triazine-2,4-diyl)bis(azanediyl))bis(3,6,9,12,15,18,21,24-octaoxaheptacosan-27-oate) (13 mg, 9.4 μmol). The reaction mixture was stirred overnight, and then the mixture was purified by prep-HPLC on a C18 column (10-70% MeCN/0.1% TFA (aq.)) to afford a mixture of the title compounds (34 mg) as TFA salts. 1-((4-(((6S,9S)-1-Amino-6-((4-((((R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carbonyl)oxy)methyl)phenyl)carbamoyl)-9-isopropyl-1,8,11-trioxo-14,17,20,23,26,29,32,35-octaoxa-2,7,10-triazaheptatriacontan-37-yl)amino)-6-(2-(2-((tert-butoxycarbonyl)amino)ethoxy)ethoxy)-1,3,5-triazin-2-yl)amino)-3,6,9,12,15,18,21,24-octaoxaheptacosan-27-oic acid. LCMS calc. for C₁₁₇H₁₈₁N₂₁O₃₄S [M+2H]²⁺ m/z=1228.6; Found 1228.5. ((((295,29'S,32S,32'S)-1,1′-((6-(2-(2-((tert-butoxycarbonyl)amino)ethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate) LCMS calc. for C₁₈₄H₂₆₆N₃₆O₄₄S₂[M+2H]2+m/z=1875.0; Found 1874.8.

### Step 7. 1-((4-(((6S,9S)-1-Amino-6-((4-((((R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carbonyl)oxy)methyl)phenyl)carbamoyl)-9-isopropyl-1,8,11-trioxo-14,17,20,23,26,29,32,35-octaoxa-2,7,10-triazaheptatriacontan-37-yl)amino)-6-(2-(2-aminoethoxy)ethoxy)-1,3,5-triazin-2-yl)amino)-3,6,9,12,15,18,21,24-octaoxaheptacosan-27-oic acid and ((((29S,29'S,32S,32'S)-1,1′-((6-(2-(2-aminoethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate)

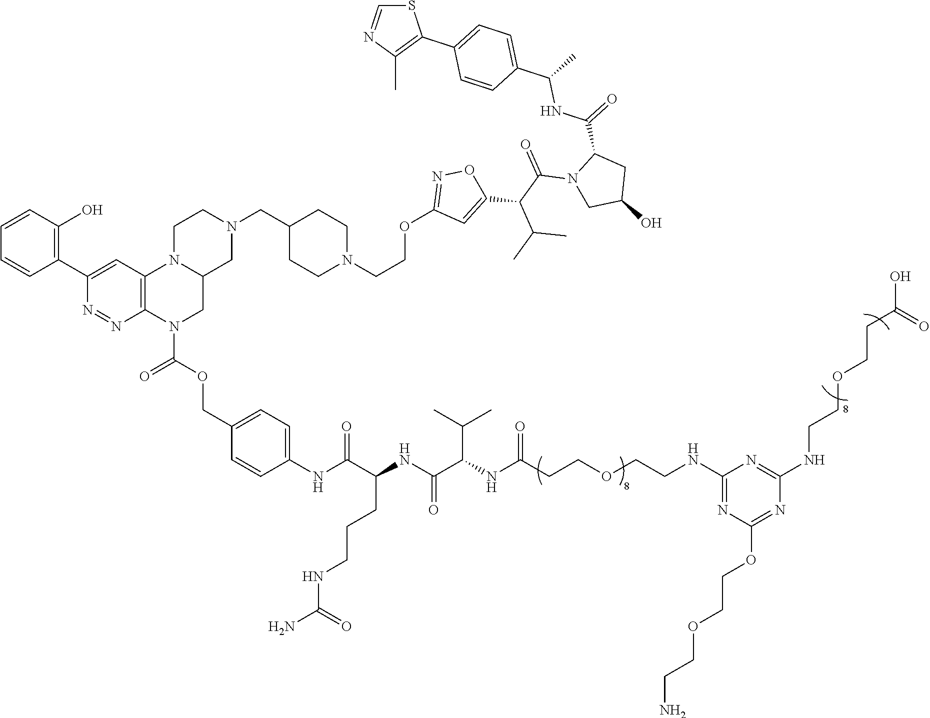

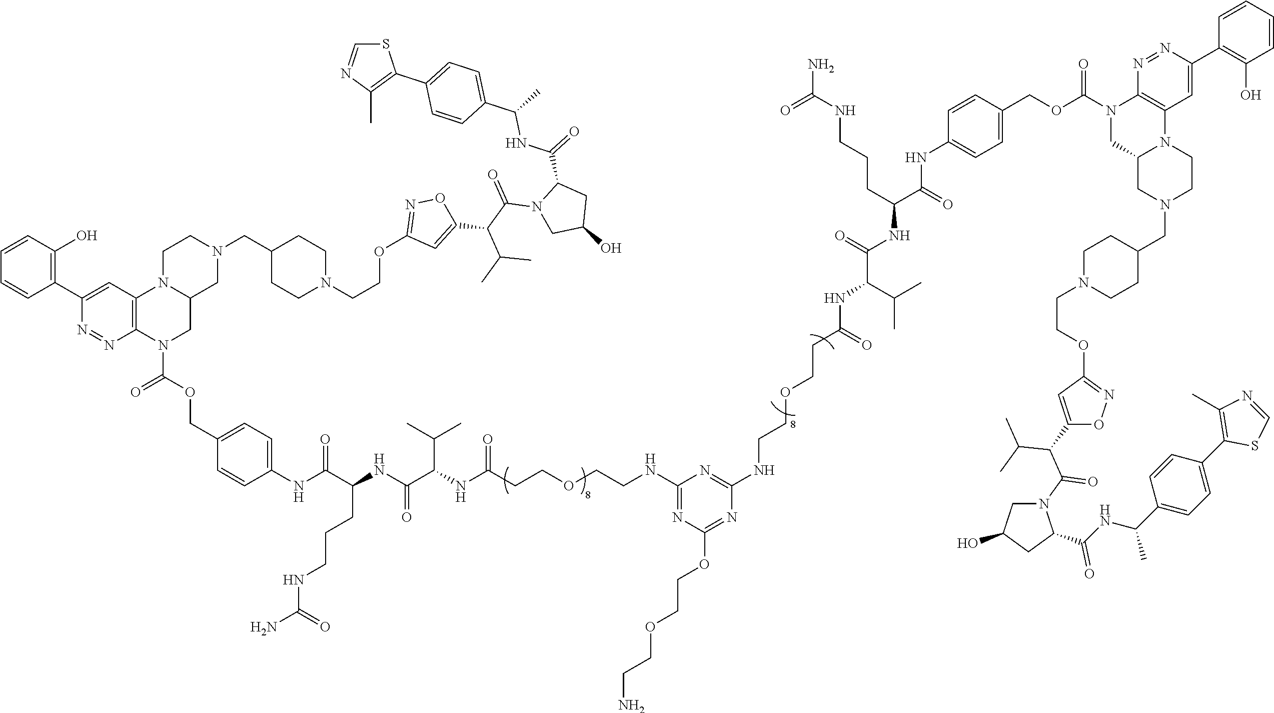

To a solution of the mixture isolated in Step 6 (34 mg) in THE (1.0 mL) was added 6 M HCl (aq.) (1.0 mL, 6.0 mmol). The reaction mixture was stirred for 2 h. The mixture was diluted with water (3.0 mL) and purified by prep-HPLC on a C18 column (15-70% MeCN/0.1% TFA (aq.)) to afford the TFA salt of 1-((4-(((6S,9S)-1-amino-6-((4-((((R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carbonyl)oxy)methyl)phenyl)carbamoyl)-9-isopropyl-1,8,11-trioxo-14,17,20,23,26,29,32,35-octaoxa-2,7,10-triazaheptatriacontan-37-yl)amino)-6-(2-(2-aminoethoxy)ethoxy)-1,3,5-triazin-2-yl)amino)-3,6,9,12,15,18,21,24-octaoxaheptacosan-27-oic acid (7.5 mg, 3.2 μmol) as a colorless oil. LCMS calc. for C₁₁₂H₁₇₃N₂₁O₃₂S [M+2H]2+m/z=1178.2; Found 1178.5. And the TFA salt of ((((29S,29'S,32S,32'S)-1,1′-((6-(2-(2-aminoethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate) (8.7 mg, 2.4 μmol) as a colorless oil. LCMS calc. for C₁₈₀H₂₅₈N₃₆O₄₂S₂[M+2H]2+m/z=1824.9; Found 1825.1.

### Step 8. ((((29S,29'S,32S,32'S)-1,1′-((6-(2-(2-(2-(2,5-Dioxo-2,5-dihydro-1H-pyrrol-1-yl)acetamido)ethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate)

To a solution of ((((29S,29'S,32S,32'S)-1,1′-((6-(2-(2-aminoethoxy)ethoxy)-1,3,5-triazine-2,4-diyl)bis(azanediyl))bis(29-isopropyl-27,30-dioxo-32-(3-ureidopropyl)-3,6,9,12,15,18,21,24-octaoxa-28,31-diazatritriacontan-33-oyl))bis(azanediyl))bis(4,1-phenylene))bis(methylene) (6aR,6a′R)-bis(8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carboxylate) (8.7 mg, 2.4 μmol) in DCM (2.0 mL) was added N,N-diisopropylethylamine (6.2 μL, 36 μmol) and 2,5-dioxopyrrolidin-1-yl 2-(2,5-dioxo-2,5-dihydro-1H-pyrrol-1-yl)acetate (1.8 mg, 7.2 μmol). The reaction mixture was stirred for 1 h. The mixture was diluted with DMSO (2.0 mL) and purified by prep-HPLC on a C18 column (15-70% MeCN/0.1% TFA (aq.)) to afford the TFA salt of the title compound (5.1 mg, 1.3 μmol, 54% yield) as a colorless oil. LCMS calc. for C₁₈₅H₂₆₁N₃₇O₄₅S₂ [M+2H]2+m/z=1893.4; Found 1893.1.

### Example 14. Synthesis of Compound 2.58—1-((4-(((6S,9S)-1-Amino-6-((4-((((R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carbonyl)oxy)methyl)phenyl)carbamoyl)-9-isopropyl-1,8,11-trioxo-14,17,20,23,26,29,32,35-octaoxa-2,7,10-triazaheptatriacontan-37-yl)amino)-6-(2-(2-(2-(2,5-dioxo-2,5-dihydro-1H-pyrrol-1-yl)acetamido)ethoxy)ethoxy)-1,3,5-triazin-2-yl)amino)-3,6,9,12,15,18,21,24-octaoxaheptacosan-27-oic acid

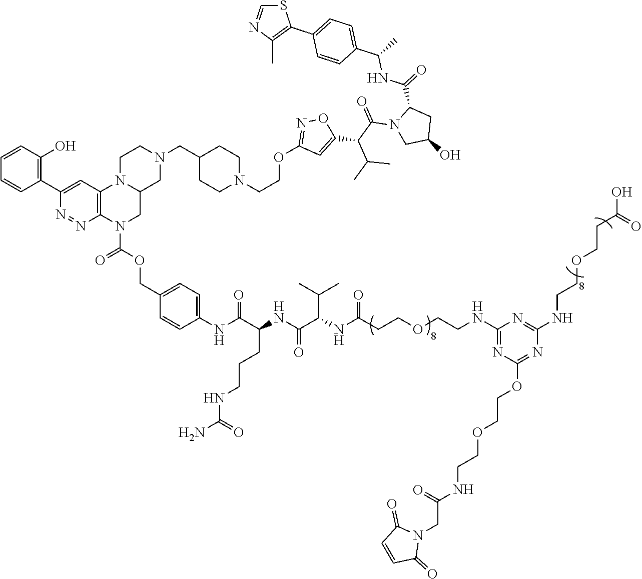

The title compound was synthesized by procedures analogous to those outlined in Example 13, Step 8 from 1-((4-(((6S,9S)-1-amino-6-((4-((((R)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-hydroxyphenyl)-6,6a,7,8,9,10-hexahydro-5H-pyrazino[1′,2′:4,5]pyrazino[2,3-c]pyridazine-5-carbonyl)oxy)methyl)phenyl)carbamoyl)-9-isopropyl-1,8,11-trioxo-14,17,20,23,26,29,32,35-octaoxa-2,7,10-triazaheptatriacontan-37-yl)amino)-6-(2-(2-aminoethoxy)ethoxy)-1,3,5-triazin-2-yl)amino)-3,6,9,12,15,18,21,24-octaoxaheptacosan-27-oic acid (see Example 13, Step 7). LCMS calc. for C₁₁₈H₁₇₆N₂₂O₃₅5 [M+2H]2+m/z=1247.1; Found 1247.5.

### Example 15. Synthesis of Compound 2.10—4-((S)-2-((S)-2-(6-(2,5-Dioxo-2,5-dihydro-1H-pyrrol-1-yl)hexanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-hydroxyphenyl)-6a,7,8,9-tetrahydropyrrolo[1′,2′:4,5]pyrazino[2,3-c]pyridazine-5(6H)-carboxylate

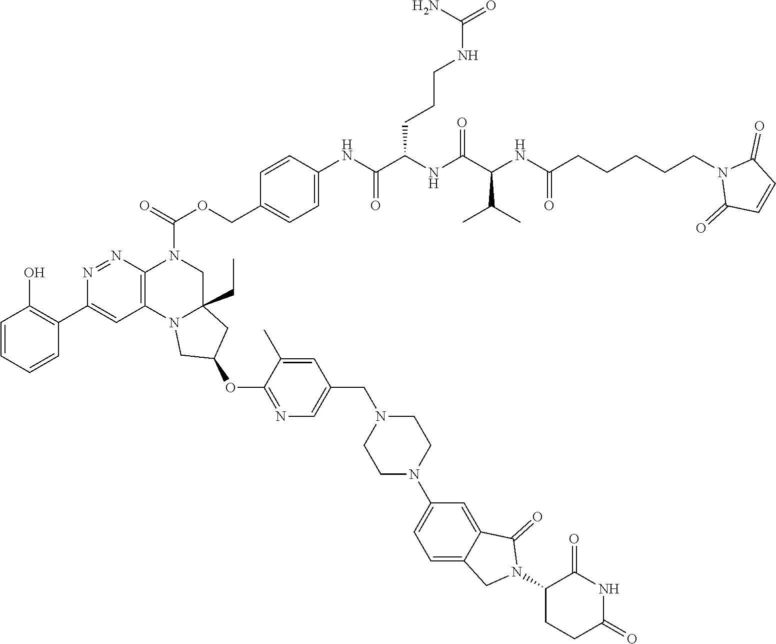 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3O)nnc1N(C(=O)OCc1ccc(NC(=O)[C@H](CCCNC(N)=O)NC(=O)[C@@H](NC(=O)CCCCCN3C(=O)C=CC3=O)C(C)C)cc1)C2]

The title compound was synthesized by procedures analogous to those outlined in Example 6, Steps 1-6, substituting (6aS,8R)-2-chloro-6a-ethyl-5,6,6a,7,8,9-hexahydropyrrolo[1′,2′:4,5]pyrazino[2,3-c]pyridazin-8-ol for (6aS,8R)-2-chloro-6a-methyl-5,6,6a,7,8,9-hexahydropyrrolo[1′,2′:4,5]pyrazino[2,3-c]pyridazin-8-ol in Step 1. LCMS calc. for C₇₀H₈₅N₁₅O₁₃ [M+2H]2+m/z=671.8; Found 672.2.

### Example 16. Synthesis of Compound 2.08—4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-hydroxyphenyl)-6a,7,8,9-tetrahydropyrrolo[1′,2′:4,5]pyrazino[2,3-c]pyridazine-5(6H)-carboxylate

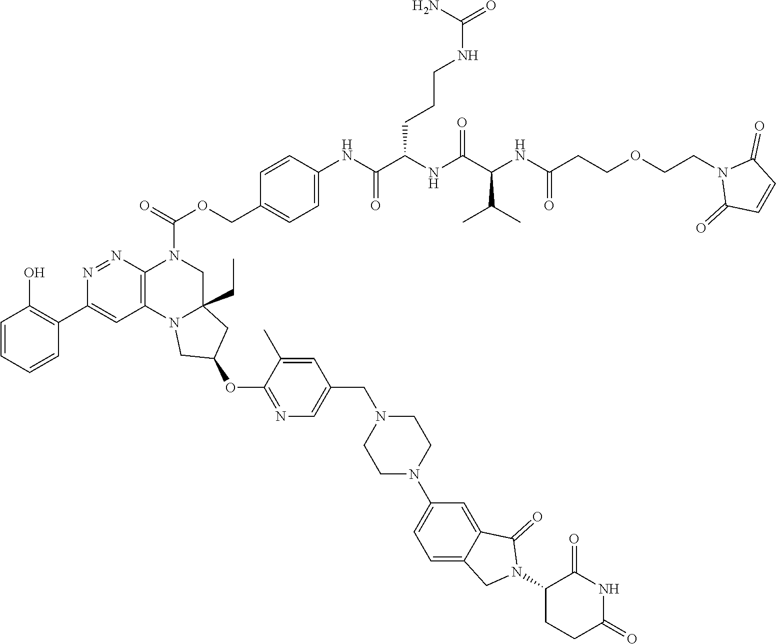 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3O)nnc1N(C(=O)OCc1ccc(NC(=O)[C@H](CCCNC(N)=O)NC(=O)[C@@H](NC(=O)CCOCCN3C(=O)C=CC3=O)C(C)C)cc1)C2]

The title compound was synthesized by procedures analogous to those outlined in Example 7, steps 1-4, substituting (6aS,8R)-8-((5-(1,3-dioxolan-2-yl)pyridin-2-yl)oxy)-6a-ethyl-2-(2-(methoxymethoxy)phenyl)-5,6,6a,7,8,9-hexahydropyrrolo[1′,2′:4,5]pyrazino[2,3-c]pyridazine for ((6aS,8R)-8-((5-(1,3-dioxolan-2-yl)pyridin-2-yl)oxy)-2-(2-(methoxymethoxy)phenyl)-6a-methyl-5,6,6a,7,8,9-hexahydropyrrolo[1′,2′:4,5]pyrazino[2,3-c]pyridazine in Step 1. LCMS calc. for C₆₉H₈₃N₁₅O₁₄ [M+2H]2+m/z=672.8; Found 673.1.

### Example 17. Synthesis of Compound 2.59 —(2S,4R)-1-((R)-2-(3-(2-(4-(((R)-5-((S)-7-benzyl-20-(2,5-dioxo-2,5-dihydro-1H-pyrrol-1-yl)-3,6,9,12,15-pentaoxo-2,5,8,11,14-pentaazaicosyl)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

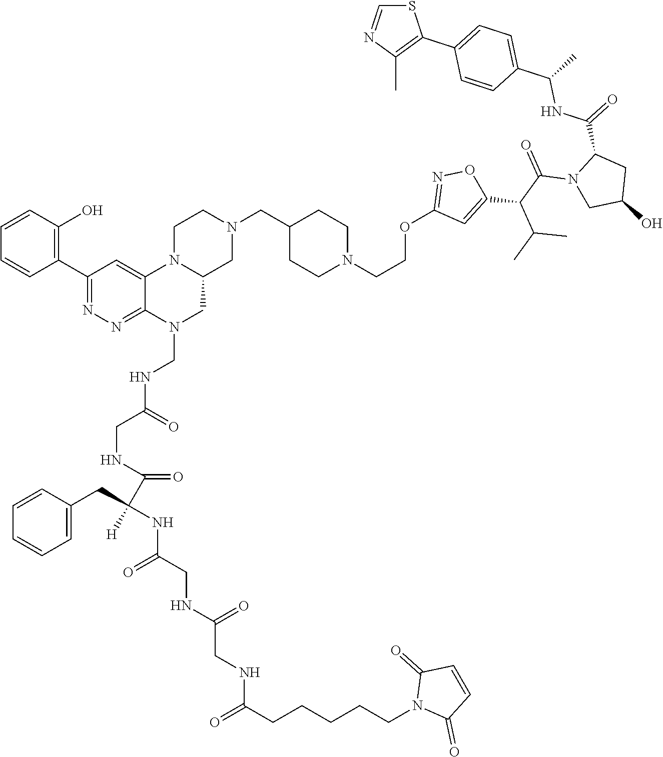 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6N(CNC(=O)CNC(=O)[C@H](Cc6ccccc6)NC(=O)CNC(=O)CNC(=O)CCCCCN6C(=O)C=CC6=O)C[C@H]5C4)CC3)no2)C(C)C)cc1]

### Step 1: (9H-Fluoren-9-yl)methyl (2-((((R)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazin-5-yl)methyl)amino)-2-oxoethyl)carbamate

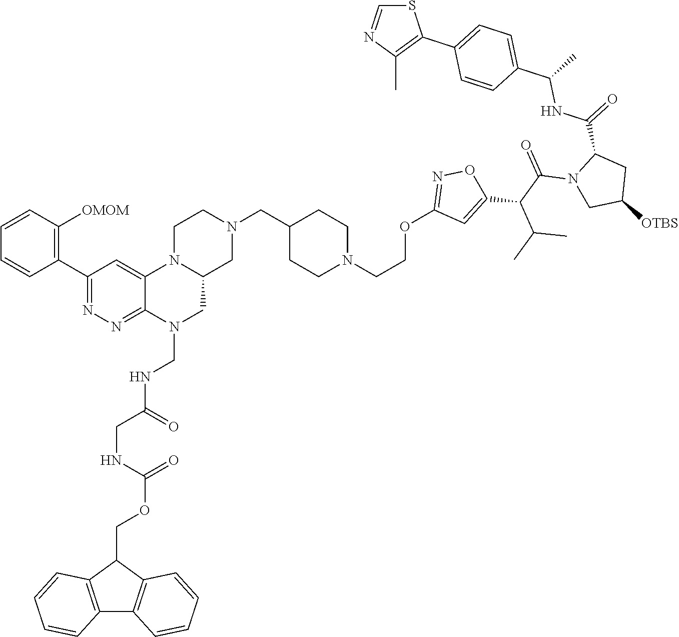

To a solution of (2S,4R)-4-((tert-butyldimethylsilyl)oxy)-1-((R)-2-(3-(2-(4-(((S)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (20 mg, 0.018 mmol) in DMF (1 mL) was added 2,6-lutidine (0.022 mL, 0.18 mmol) at 0° C. The resulting mixture was stirred for 30 min. (9H-Fluoren-9-yl)methyl (2-((chloromethyl)amino)-2-oxoethyl)carbamate (25.9 mg, 0.0752 mmol) was added. The reaction mixture was stirred for 15 h at 25° C. The reaction mixture was diluted with MeCN and purified by prep-HPLC on a C18 column (34.4-54.4% MeCN/0.2% TFA (aq.)) to give the title compound as a TFA salt (8.0 mg, 0.0058 mmol, 31% yield). LCMS calc. for C₆₈H₈₁N₁₅O₁₃ [M+2H]²⁺ m/z=686.3; Found 686.4.

### Step 2: (2S,4R)-1-((R)-2-(3-(2-(4-(((R)-5-((2-Aminoacetamido)methyl)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide

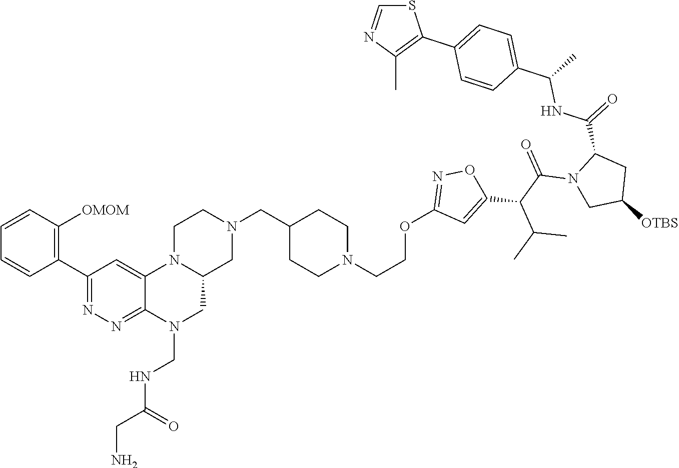

To a solution of (9H-fluoren-9-yl)methyl (2-((((R)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-2-(2-(methoxymethoxy)phenyl)-6,6a,7,8,9,10-hexahydro-5H-pyrazino[1′,2′:4,5]pyrazino[2,3-c]pyridazin-5-yl)methyl)amino)-2-oxoethyl)carbamate (8.0 mg, 0.0058 mmol) in DMF (1 mL) was added piperidine (5.8 μL, 0.058 mmol). The reaction mixture was stirred for 3 h. The reaction mixture was diluted with MeCN and purified by prep-HPLC on a C18 column (24.2-44.2% MeCN/0.2% TFA (aq.)) to give the title compound as a TFA salt (6 mg, 0.005 mmol, 90% yield). LCMS calc. for C₅₉H₈₅N₁₂O₈SSi [M+H]⁺m/z=1149.6; Found 1149.6.

### Step 3: (2S,4R)-1-((R)-2-(3-(2-(4-(((R)-5-((S)-7-Benzyl-20-(2,5-dioxo-2,5-dihydro-1H-pyrrol-1-yl)-3,6,9,12,15-pentaoxo-2,5,8,11,14-pentaazaicosyl)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide

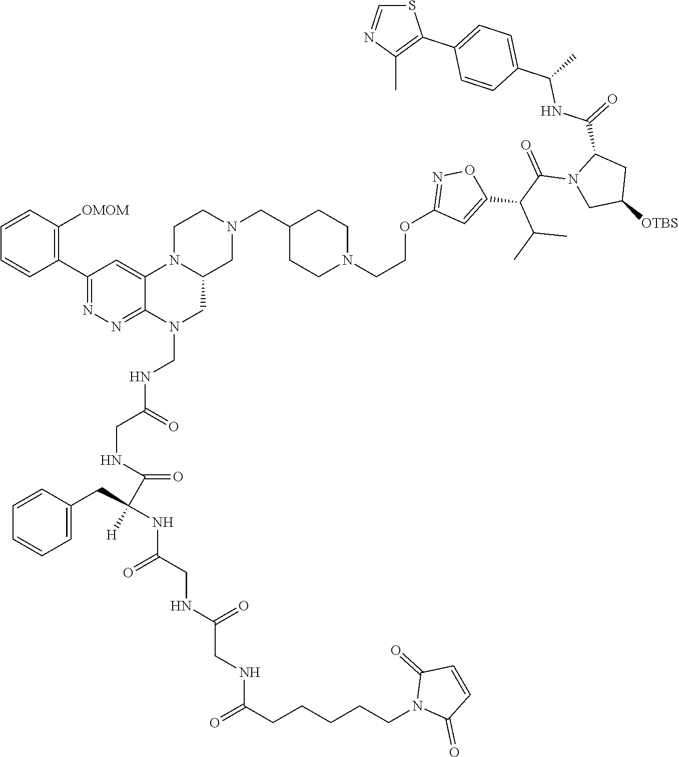

A solution of(6-(2,5-dioxo-2,5-dihydro-1H-pyrrol-1-yl)hexanoyl)glycylglycyl-D-phenylalanine (4.9 mg, 0.010 mmol), 1-hydroxybenzotriazole hydrate (1.8 mg, 0.013 mmol), and 1-[bis(dimethylamino)methylene]-1H-1,2,3-triazolo[4,5-b]pyridinium 3-oxid hexafluorophosphate(5.0 mg, 0.013 mmol) in DMF (1 mL) was stirred for 1 h. To the resulting solution was added (2S,4R)-1-((R)-2-(3-(2-(4-(((R)-5-((2-aminoacetamido)methyl)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (6 mg, 0.005 mmol) and N,N-diisopropylethylamine (5 μL, 0.03 mmol). The reaction mixture was stirred for 3 h. The reaction mixture was diluted with MeCN and purified by prep-HPLC on a C18 column (29.7-49.7% MeCN/0.2% TFA (aq.)) to give the title compound as a TFA salt (4.1 mg, 0.0025 mmol, 48% yield). LCMS calc. for C₈₂H₁₁₂N₁₆O₁₄SSi [M+2H]²⁺ m/z=802.4; Found 802.4.

### Step 4: (2S,4R)-1-((R)-2-(3-(2-(4-(((R)-5-((S)-7-Benzyl-20-(2,5-dioxo-2,5-dihydro-1H-pyrrol-1-yl)-3,6,9,12,15-pentaoxo-2,5,8,11,14-pentaazaicosyl)-2-(2-hydroxyphenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-4-hydroxy-N—((S)-1-(4-(4-methylthiazol-5-yl)phenyl)ethyl)pyrrolidine-2-carboxamide

The title compound was synthesized by procedures analogous to those outlined in Example 3, step 6. LCMS calc. for C₇₄H₉₄N₁₆O₁₃S [M+2H]2+m/z=723.3; Found 723.4.

### Example 18. Synthesis of Compound 2.60 —N-(5-(2,5-Dioxo-2,5-dihydro-1H-pyrrol-1-yl)pentyl)-N—((S)-1-((4-((2-((S)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)cyclobutane-1,1-dicarboxamide

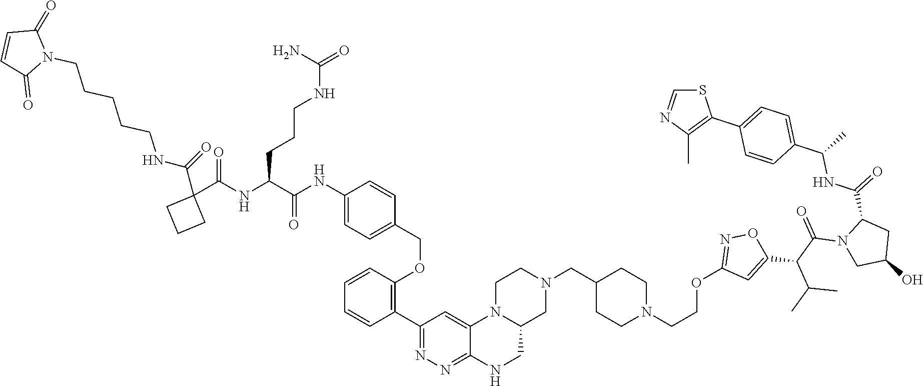 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7OCc7ccc(NC(=O)[C@H](CCCNC(N)=O)NC(=O)C8(C(=O)NCCCCCN9C(=O)C=CC9=O)CCC8)cc7)nnc6NC[C@H]5C4)CC3)no2)C(C)C)cc1]

### Step 1: 1—(((S)-1-((4-((2-((S)-8-((1-(tert-Butoxycarbonyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid

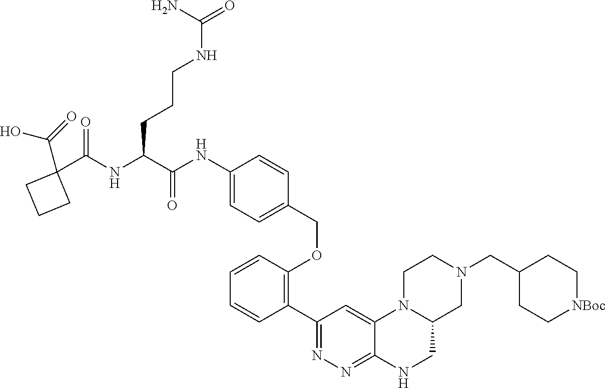 [SMILES: CC(C)(C)OC(=O)N1CCC(CN2CCN3c4cc(-c5ccccc5OCc5ccc(NC(=O)[C@H](CCCNC(N)=O)NC(=O)C6(C(=O)O)CCC6)cc5)nnc4NC[C@H]3C2)CC1]

To a solution of crude ethyl 1-[[(2S)-5-(carbamoylamino)-1-oxo-1-[4-[[2-(4,4,5,5-tetramethyl-1,3,2-dioxaborolan-2-yl)phenoxy]methyl]anilino]pentan-2-yl]carbamoyl]cyclobutane-1-carboxylate (200 mg) (prepared by following the procedures reported in WO2022/020288) in DMSO (4 mL) was added chloro[(di(1-adamantyl)-N-butylphosphine)-2-(2-aminobiphenyl)]palladium(II) (20 mg, 0.03 mmol), tert-butyl 4-[[(10S)-4-chloro-1,5,6,8,12-pentazatricyclo[8.4.0.02,7]tetradeca-2,4,6-trien-12-yl]methyl]piperidine-1-carboxylate (126 mg, 0.299 mmol), K₃PO₄ (190 mg, 0.90 mmol) and water (0.3 mL). The mixture was then bubbled with N₂ for 5 min. The reaction mixture was then stirred at 100° C. in a sealed tube for 12 h. The mixture was then cooled to room temperature, filtered, and purified by prep-HPLC on a C18 column (5-60% MeCN/0.05% formic acid (aq.)) to afford the title compound as a formic acid salt (30 mg, 0.035 mmol, 12% yield). LCMS calc. for C₄₅H₆₁N₁₀O₈ [M+H]⁺m/z=869.5; Found 869.2.

### Step 2: 1—(((S)-1-Oxo-1-((4-((2-((S)-8-(piperidin-4-ylmethyl)-6,6a,7,8,9,10-hexahydro-5H-pyrazino[1′,2′:4,5]pyrazino[2,3-c]pyridazin-2-yl)phenoxy)methyl)phenyl)amino)-5-ureidopentan-2-yl)carbamoyl)cyclobutane-1-carboxylic acid

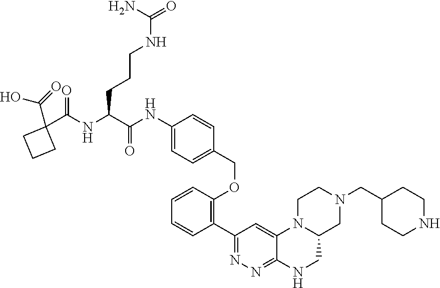 [SMILES: NC(=O)NCCC[C@H](NC(=O)C1(C(=O)O)CCC1)C(=O)Nc1ccc(COc2ccccc2-c2cc3c(nn2)NC[C@H]2CN(CC4CCNCC4)CCN32)cc1]

To a solution of 1-(((S)-1-((4-((2-((S)-8-((1-(tert-butoxycarbonyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid (30 mg, 0.035 mmol) in DCM (2 mL) was added TFA (1 mL). The mixture was stirred for 2 h. The mixture was concentrated and used directly in the next step without any further purification. LCMS calc. for C₄₀H₅₃N₁₀O₆[M+H]⁺m/z=769.4; Found 769.5.

### Step 3: 1—(((S)-1-((4-((2-((S)-8-((1-(2-((5-((R)-1-((2S,4R)-4-((tert-Butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid

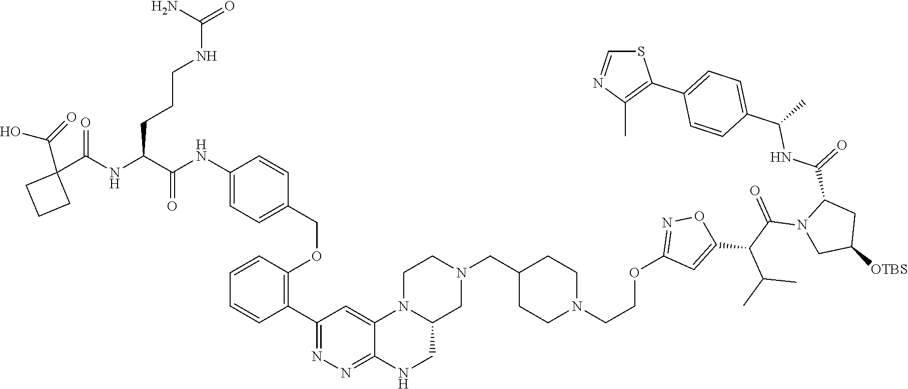 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCN3CCC(CN4CCN5c6cc(-c7ccccc7OCc7ccc(NC(=O)[C@H](CCCNC(N)=O)NC(=O)C8(C(=O)O)CCC8)cc7)nnc6NC[C@H]5C4)CC3)no2)C(C)C)cc1]

To a solution of (2S,4R)-4-[tert-butyl(dimethyl)silyl]oxy-1-[(2R)-3-methyl-2-[3-(2-oxoethoxy)-1,2-oxazol-5-yl]butanoyl]-N-[(1S)-1-[4-(4-methyl-1,3-thiazol-5-yl)phenyl]ethyl]pyrrolidine-2-carboxamide (40 mg, 0.06 mmol) and crude 1-(((S)-1-Oxo-1-((4-((2-((S)-8-(piperidin-4-ylmethyl)-6,6a,7,8,9,10-hexahydro-5H-pyrazino[1′,2′:4,5]pyrazino[2,3-c]pyridazin-2-yl)phenoxy)methyl)phenyl)amino)-5-ureidopentan-2-yl)carbamoyl)cyclobutane-1-carboxylic acid (40 mg) in DCM (2 mL) and methanol (2 mL) at 0° C. was added sodium cyanoborohydride (6.4 mg, 0.17 mmol) and then one drop of formic acid. The mixture was stirred at room temperature for 24 h and then purified by prep-HPLC on a C18 column (5-50% MeCN/0.05% formic acid (aq.)) to afford the title compound as a formic acid salt (20 mg, 0.14 mmol, 28% yield). LCMS calc. for C₇₃H₉₉N₁₄O₁₁SSi [M+H]⁺m/z=1407.7; Found 1407.9.

### Step 4: N—((S)-1-((4-((2-((S)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)-N-(5-(2,5-dioxo-2,5-dihydro-1H-pyrrol-1-yl)pentyl)cyclobutane-1,1-dicarboxamide

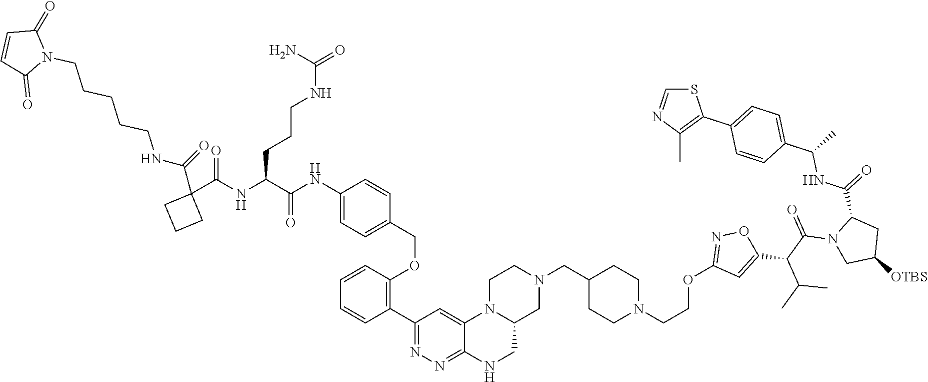 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCN3CCC(CN4CCN5c6cc(-c7ccccc7OCc7ccc(NC(=O)[C@H](CCCNC(N)=O)NC(=O)C8(C(=O)NCCCCCN9C(=O)C=CC9=O)CCC8)cc7)nnc6NC[C@H]5C4)CC3)no2)C(C)C)cc1]

To a solution of 1-(((S)-1-((4-((2-((S)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid (20 mg, 0.014 mmol) in DMF (1 mL) was added 1-ethyl-3-(3-dimethylaminopropyl)carbodiimide hydrochloride (4 mg, 0.02 mmol), 1-hydroxybenzotriazole hydrate (2.4 mg, 0.018 mmol), 1-(5-aminopentyl)pyrrole-2,5-dione (7.8 mg, 0.036 mmol) and N,N-diisopropylethylamine (9.2 mg, 0.071 mmol). The mixture was stirred for 12 h and purified by prep-HPLC on C18 column (5-50% MeCN/0.05% formic acid (aq.)) to afford the title compound as a formic acid salt (9.1 mg, 0.0058 mmol, 41% yield). LCMS calc. for C₈₂H₁₁₁N₁₆O₁₂SSi [M+H]⁺m/z=1571.8; Found 1572.0.

### Step 5: N-(5-(2,5-Dioxo-2,5-dihydro-1H-pyrrol-1-yl)pentyl)-N—((S)-1-((4-((2-((S)-8-((1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)cyclobutane-1,1-dicarboxamide

To a solution of N—((S)-1-((4-((2-((S)-8-((1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)methyl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)-N-(5-(2,5-dioxo-2,5-dihydro-1H-pyrrol-1-yl)pentyl)cyclobutane-1,1-dicarboxamide (9 mg, 0.006 mmol) in isopropyl alcohol (1 mL) and THE (1 mL) was added conc. HCl (0.5 m1). The mixture was stirred for 15 min and purified by prep-HPLC on a C₁₈ column (5-50% MeCN/0.05% formic acid (aq.)) to afford the title compound as a formic acid salt (4.5 mg, 0.0031 mmol, 50% yield). LCMS calc. for C₇₆H₉₇N₁₆O₁₂S [M+H]⁺m/z=1457.7; Found 1457.7.

### Example 19. Synthesis of Compound 2.61 —N—((S)-7-Benzyl-1-((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-hydroxyphenyl)-6a,7,8,9-tetrahydropyrrolo[1′,2′:4,5]pyrazino[2,3-c]pyridazin-5(6H)-yl)-3,6,9,12-tetraoxo-2,5,8,11-tetraazatridecan-13-yl)-6-(2,5-dioxo-2,5-dihydro-1H-pyrrol-1-yl)hexanamide

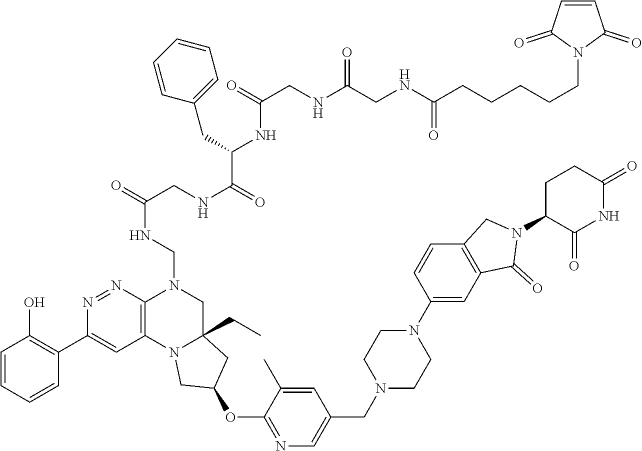 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3O)nnc1N(CNC(=O)CNC(=O)[C@H](Cc1ccccc1)NC(=O)CNC(=O)CNC(=O)CCCCCN1C(=O)C=CC1=O)C2]

### Step 1: 1-Bromo-2-((2-methoxyethoxy)methoxy)benzene

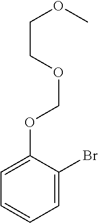

Sodium tert-butoxide (3.92 g, 40.8 mmol) was added to a solution of 2-bromophenol (6.41 g, 37.1 mmol) in anhydrous THE (100 mL) at 0° C. The mixture was stirred at 0° C. for 5 min then 1-((chloromethoxy)methoxy)-2-methoxyethane (5.16 mL, 40.8 mmol) was added drop wise. The ice bath was removed, the mixture was allowed to warm to room temperature, and stirred for 30 min. The reaction mixture was diluted with brine (30 mL) and extracted with ethyl acetate (2×30 mL). The combined organic phase was dried over anhydrous magnesium sulfate, filtered, and concentrated. The crude material was purified by silica gel chromatography (0-20% EtOAc/hexanes) to afford the title compound (9.01 g, 93% yield), as colorless oil. ¹H NMR (400 MHz, CDCl₃) δ 7.54 (dd, J=7.9, 1.6 Hz, 1H), 7.27-7.19 (m, 2H), 6.90 (td, J=7.6, 1.7 Hz, 1H), 5.35 (s, 2H), 3.94-3.83 (m, 2H), 3.62-3.51 (m, 2H), 3.38 (s, 3H).

### Step 2: 2-(2-((2-Methoxyethoxy)methoxy)phenyl)-4,4,5,5-tetramethyl-1,3,2-dioxaborolane

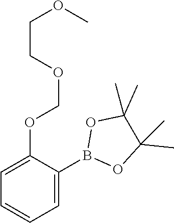 [SMILES: COCCOCOc1ccccc1B1OC(C)(C)C(C)(C)O1]

n-Butyllithium (16.3 mL, 40.7 mmol, 2.5 M in hexanes) was added drop wise to a solution of 1-bromo-2-(2-methoxyethoxymethoxy)benzene (8.85 g, 33.9 mmol) in anhydrous THE (100 mL) at −78° C. The mixture was stirred at −78° C. for 30 min then 2-isopropoxy-4,4,5,5-tetramethyl-1,3,2-dioxaborolane (8.30 mL, 40.7 mmol) was added drop wise. The resulting mixture was allowed to warm to room temperature and stirred for 3 h. The mixture was diluted by slow addition of water (50 mL) and extracted with ethyl acetate (2×100 mL). The combined organic phase was dried over anhydrous magnesium sulfate, filtered, and concentrated. The crude material was purified by silica gel chromatography (0-15% EtOAc/hexanes) to afford the title compound (8.20 g, 78% yield) as clear oil. ¹H NMR (400 MHz, CDCl₃) δ 7.71 (dd, J=7.3, 1.8 Hz, 1H), 7.40 (ddd, J=8.4, 7.3, 1.9 Hz, 1H), 7.16-6.98 (m, 2H), 5.31 (s, 2H), 3.94-3.85 (m, 2H), 3.65-3.52 (m, 2H), 3.39 (s, 3H), 1.36 (s, 12H).

### Step 3: (6aS,8R)-6a-Ethyl-2-(2-((2-methoxyethoxy)methoxy)phenyl)-5,6,6a,7,8,9-hexahydropyrrolo[1′,2′:4,5]pyrazino[2,3-c]pyridazin-8-ol

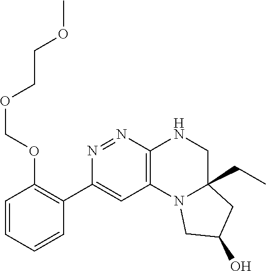 [SMILES: CC[C@]12CNc3nnc(-c4ccccc4OCOCCOC)cc3N1C[C@H](O)C2]

Chloro(2-dicyclohexylphosphino-2′,6′-dimethoxy-1,1′-biphenyl)[2-(2′-amino-1,1′-biphenyl)]palladium(II) (SPhos Pd G2) (322 mg, 0.450 mmol), 2-[2-(2-methoxyethoxymethoxy)phenyl]-4,4,5,5-tetramethyl-1,3,2-dioxaborolane (1.66 g, 5.37 mmol), and potassium carbonate (1.24 g, 8.95 mmol) were added to a solution of (6aS,8R)-2-chloro-6a-ethyl-5,6,6a,7,8,9-hexahydropyrrolo[1′,2′:4,5]pyrazino[2,3-c]pyridazin-8-ol (1.14 g, 4.48 mmol, Example 1, Compound 1A, Step 6) in 1,4-dioxane (20 mL) and water (4.0 mL). The mixture was sparged with nitrogen for 5 min then heated to 90° C. for 6 h. The reaction mixture was cooled to room temperature and extracted with a solution of DCM/MeOH (6:1 v/v) (3×40 mL). The combined organic phase was dried over anhydrous magnesium sulfate, filtered, and concentrated. The crude residue purified by silica gel chromatography (0-25% MeOH/DCM) to afford the title compound (1.02 g, 57% yield), as white solid. LCMS calc. for C₂₁H₂₉N₄O₄ [M+H]⁺: m/z=401.2; Found: 401.1.

### Step 4: (6aS,8R)-8-((5-(1,3-dioxolan-2-yl)-3-methylpyridin-2-yl)oxy)-6a-ethyl-2-(2-((2-methoxyethoxy)methoxy)phenyl)-5, 6,6a,7,8,9-hexahydropyrrolo[1′,2′:4,5]pyrazino[2,3-c]pyridazine

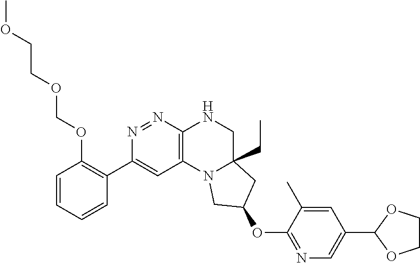 [SMILES: CC[C@]12CNc3nnc(-c4ccccc4OCOCCOC)cc3N1C[C@H](Oc1ncc(C3OCCO3)cc1C)C2]

5-(1,3-Dioxolan-2-yl)-2-fluoro-3-methylpyridine (458 mg, 2.50 mmol, Example 25, Step 2) and sodium hydride (150 mg, 3.75 mmol, 60% in mineral oil) were sequentially added to a solution of (6aS,8R)-6a-ethyl-2-(2-((2-methoxyethoxy)methoxy)phenyl)-5,6,6a,7,8,9-hexahydropyrrolo[1′,2′:4,5]pyrazino[2,3-c]pyridazin-8-ol (0.500 g, 1.25 mmol) in anhydrous THE (4.0 mL) and DMF (2.0 mL) at 0° C. The reaction was allowed to warm up to room temperature and stirred for 3 h. The mixture was quenched by slow addition of cold brine (10 mL) and extracted with a solution of DCM/MeOH (6:1 v/v) (3×40 mL). The combined organic phase was dried over anhydrous magnesium sulfate, filtered, and concentrated. The crude material was purified by silica gel chromatography (0-15% MeOH/DCM) to afford the title compound (522 mg, 74% yield) as a pale yellow solid. LCMS calc. for C₃₀H₃₈N₅O₆ [M+H]⁺: m/z=564.3; Found: 564.1.

### Step 5: (9H-fluoren-9-yl)methyl (2-((((6aS,8R)-6a-ethyl-8-((5-formyl-3-methylpyridin-2-yl)oxy)-2-(2-((2-methoxyethoxy)methoxy)phenyl)-6a,7,8,9-tetrahydropyrrolo[1′,2′:4,5]pyrazino[2,3-c]pyridazin-5(6H)-yl)methyl)amino)-2-oxoethyl)carbamate

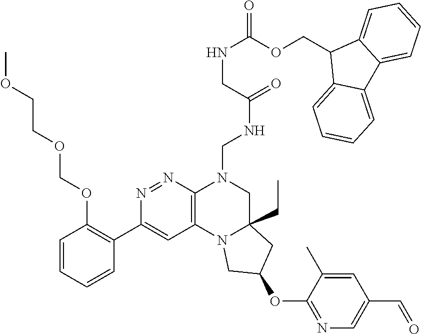 [SMILES: CC[C@@]12C[C@@H](Oc3ncc(C=O)cc3C)CN1c1cc(-c3ccccc3OCOCCOC)nnc1N(CNC(=O)CNC(=O)OCC1c3ccccc3-c3ccccc31)C2]

(6aS,8R)-8-((5-(1,3-Dioxolan-2-yl)-3-methylpyridin-2-yl)oxy)-6a-ethyl-2-(2-((2-methoxyethoxy)methoxy)phenyl)-5, 6,6a,7,8,9-hexahydropyrrolo[1′,2′:4,5]pyrazino[2,3-c]pyridazine (130 mg, 0.230 mmol), (2-((((9H-fluoren-9-yl)methoxy)carbonyl)amino)acetamido)methyl acetate (170 mg, 0.460 mmol), and 2,6-lutidine (247 mg, 2.31 mmol) were dissolved in 1,2-dichloroethane (2.0 mL) and heated to 50° C. for 3 h. The reaction mixture was cooled to room temperature, diluted with DMSO, and purified by prep-HPLC on a C18 column ((41-61% MeCN/0.1% TFA (sq.)) to afford the TFA salt of the title compound (55.0 mg, 25% yield), as white solid. LCMS calc. for C₄₆H₅₀N₇O₈ [M+H]⁺: m/z=828.4; Found: 828.2.

### Step 6: (9H-Fluoren-9-yl)methyl (2-((((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((2-methoxyethoxy)methoxy)phenyl)-6a,7,8,9-tetrahydropyrrolo[1′,2′:4,5]pyrazino[2,3-c]pyridazin-5(6H)-yl)methyl)amino)-2-oxoethyl)carbamate

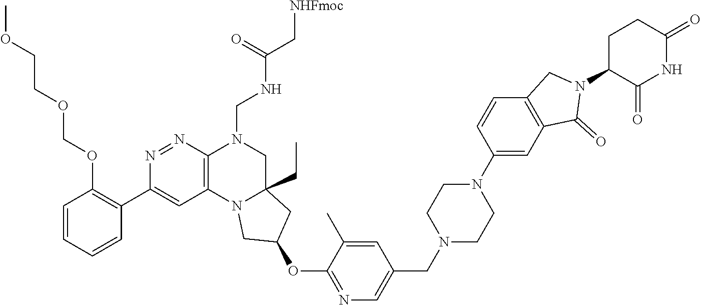

(S)-3-(1-Oxo-6-(piperazin-1-yl)isoindolin-2-yl)piperidine-2,6-dione (21.4 mg, 65.3 μmol), acetic acid (18.7 μL), and N,N-diisopropylethylamine (28.4 μL, 0.160 mmol) were dissolved in DMSO (0.50 mL) and stirred at room temperature for 15 min. (9H-Fluoren-9-yl)methyl (2-((((6aS,8R)-6a-ethyl-8-((5-formyl-3-methylpyridin-2-yl)oxy)-2-(2-((2-methoxyethoxy)methoxy)phenyl)-6a,7,8,9-tetrahydropyrrolo[1′,2′:4,5]pyrazino[2,3-c]pyridazin-5(6H)-yl)methyl)amino)-2-oxoethyl)carbamate (27.0 mg, 32.6 μmol) was added and the mixture was stirred at room temperature for 15 min. Next, sodium triacetoxyborohydride (69.1 mg, 0.330 mmol) was added and the mixture was stirred at room temperature for 5 h. The mixture was diluted with MeCN and purified by prep-HPLC on a C18 column (9-38% MeCN/0.1% TFA (aq.)) to afford the TFA salt of the title compound (9.30 mg, 23% yield), as white solid. LCMS calc. for C₆₃H₇₀N₁₁O₁₀[M+H]⁺: m/z=1140.5; Found: 1140.5.

### Step 7: 2-Amino-N-(((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((2-methoxyethoxy)methoxy)phenyl)-6a,7,8,9-tetrahydropyrrolo[1′,2′:4,5]pyrazino[2,3-c]pyridazin-5(6H)-yl)methyl)acetamide

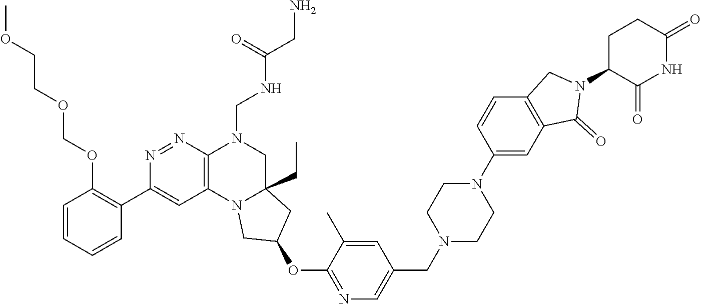 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3OCOCCOC)nnc1N(CNC(=O)CN)C2]

1,8-Diazabicyclo[5.4.0]undec-7-ene (7.85 μL, 52.6 μmol) was added to a solution of (9H-fluoren-9-yl)methyl (2-((((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((2-methoxyethoxy)methoxy)phenyl)-6a,7,8,9-tetrahydropyrrolo[1′,2′:4,5]pyrazino[2,3-c]pyridazin-5(6H)-yl)methyl)amino)-2-oxoethyl)carbamate (15.0 mg, 13.2 μmol) in anhydrous DMF(0.50 mL) and the mixture was stirred at room temperature for 15 min. The mixture was diluted with a solution of MeCN/H₂O (6/1 v/v) (4.0 mL) and purified by prep-HPLC on a C18 column (11-31% MeCN/0.1% TFA (aq.)) to afford the TFA salt of the title compound (6.50 mg, 48% yield), as white solid. LCMS calc. for C₄₈H₆₀N₁₁O₈[M+H]⁺: m/z=918.5; Found: 918.2.

### Step 8: N—((S)-7-Benzyl-1-((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((2-methoxyethoxy)methoxy)phenyl)-6a,7,8,9-tetrahydropyrrolo[1′,2′:4,5]pyrazino[2,3-c]pyridazin-5(6H)-yl)-3,6,9,12-tetraoxo-2,5,8,11-tetraazatridecan-13-yl)-6-(2,5-dioxo-2,5-dihydro-1H-pyrrol-1-yl)hexanamide

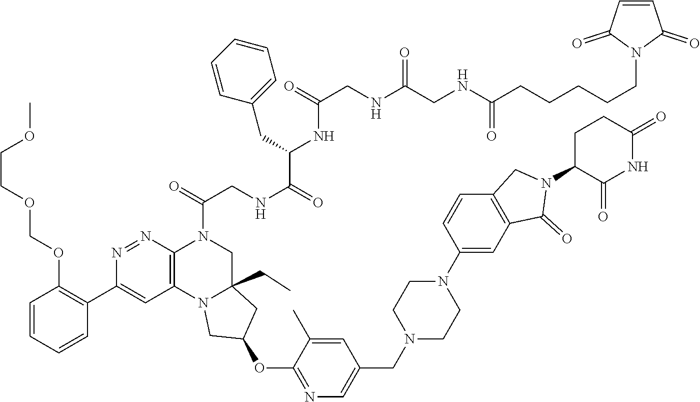 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3OCOCCOC)nnc1N(C(=O)CNC(=O)[C@H](Cc1ccccc1)NC(=O)CNC(=O)CNC(=O)CCCCCN1C(=O)C=CC1=O)C2]

(6-(2,5-Dioxo-2,5-dihydro-1H-pyrrol-1-yl)hexanoyl)glycylglycyl-L-phenylalanine (6.69 mg, 14.2 μmol), 1-hydroxybenzotriazole hydrate (2.87 mg, 18.8 μmol), 1-[bis(dimethylamino)methylene]-1H-1,2,3-triazolo[4,5-b]pyridinium 3-oxid hexafluorophosphate (HATU) (8.08 mg, 21.3 μmol) were dissolved in anhydrous DMF (0.50 mL) and the mixture was stirred at room temperature for 15 min. Next, 2-amino-N-(((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((2-methoxyethoxy)methoxy)phenyl)-6a,7,8,9-tetrahydropyrrolo[1′,2′:4,5]pyrazino[2,3-c]pyridazin-5(6H)-yl)methyl)acetamide (6.50 mg, 7.07 μmol) and N,N-diisopropylethylamine (7.40 μL, 42.5 μmol) were added to the mixture and stirred at room temperature for 10 min. The crude material was diluted with a solution of MeCn/H₂O (6/1 v/v) (4.0 mL) and purified by prep-HPLC on a C18 column (19-39% MeCN/0.1% TFA (aq.)) to afford the TFA salt of the title compound (6.20 mg, 59% yield), as white solid. LCMS calc. for C₇₁H₈₇N₁₅O₁₄ [M+2H]²⁺: m/z=686.8; Found: 687.1.

### Step 9: N—((S)-7-Benzyl-1-((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-hydroxyphenyl)-6a,7,8,9-tetrahydropyrrolo[1′,2′:4,5]pyrazino[2,3-c]pyridazin-5(6H)-yl)-3,6,9,12-tetraoxo-2,5,8,11-tetraazatridecan-13-yl)-6-(2,5-dioxo-2,5-dihydro-1H-pyrrol-1-yl)hexanamide

To a solution of N—((S)-7-benzyl-1-((6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((2-methoxyethoxy)methoxy)phenyl)-6a,7,8,9-tetrahydropyrrolo[1′,2′:4,5]pyrazino[2,3-c]pyridazin-5(6H)-yl)-3,6,9,12-tetraoxo-2,5,8,11-tetraazatridecan-13-yl)-6-(2,5-dioxo-2,5-dihydro-1H-pyrrol-1-yl)hexanamide (6.20 mg, 4.52 μmol) in isopropanol (0.50 mL) was added 6 N hydrochloric acid (aq.) (0.780 mL, 4.67 mmol). The mixture was stirred at room temperature for 1 h then diluted with MeCN and purified by prep-HPLC on a C18 column (19-39% CH₃CN/0.1% TFA (aq.)) to afford the TFA salt of the title compound (3.50 mg, 50% yield), as white solid. LCMS calc. for C₆₇H₇₉N₁₅O₁₂ [M+2H]2+: m/z=642.8; Found: 643.1.

### Example 20. Synthesis of Compound 2.62—4-((S)-2-((S)-2-(6-(2,5-Dioxo-2,5-dihydro-1H-pyrrol-1-yl)hexanamido)-3-methylbutanamido)-5-ureidopentanamido)benzyl (6S,6aS)-8-(1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

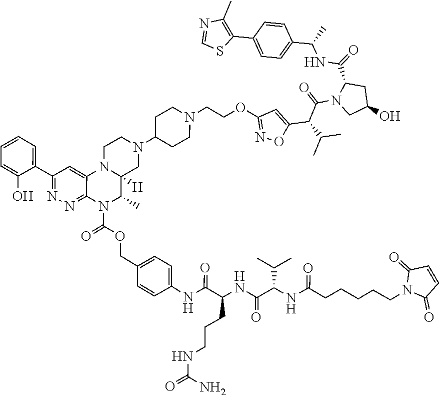 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCCCCN7C(=O)C=CC7=O)C(C)C)cc6)[C@@H](C)[C@@H]5C4)CC3)no2)C(C)C)cc1]

### Step 1: (6S,6aS)-2-Chloro-6-methyl-6,6a,7,8,9,10-hexahydro-5H-pyrazino[1′,2′:4,5]pyrazino[2,3-c]pyridazine

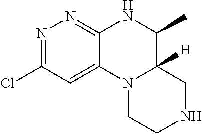 [SMILES: C[C@@H]1Nc2nnc(Cl)cc2N2CCNC[C@@H]12]

tert-Butyl (6S,6aS)-2-chloro-6-methyl-5,6,6a,7,9,10-hexahydro-8H-pyrazino[1′,2′:4,5]pyrazino[2,3-c]pyridazine-8-carboxylate (Example 2, Intermediate 4, Step 4) (50.0 mg, 0.147 mmol) was dissolved in HCl solution (3.00 mL, 12.0 mmol, 4N in 1,4-dioxane) and the mixture was stirred at room temperature for 3 h. The reaction mixture was concentrated and used in the next step without further purification. LCMS calc. for C₁₀H₁₅ClN₅[M+H]⁺: m/z=240.1; Found: 240.1.

### Step 2: tert-Butyl 4-((6S,6aS)-2-chloro-6-methyl-5,6,6a,7,9,10-hexahydro-8H-pyrazino[1′,2′:4,5]pyrazino[2,3-c]pyridazin-8-yl)piperidine-1-carboxylate

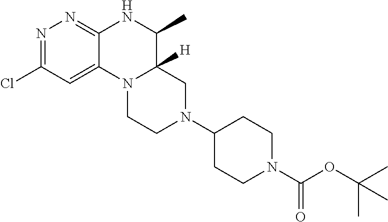 [SMILES: C[C@@H]1Nc2nnc(Cl)cc2N2CCN(C3CCN(C(=O)OC(C)(C)C)CC3)C[C@@H]12]

The title compound was synthesized by procedures analogous to those outlined in Example 3, Step 1 substituting 1-Boc-Piperidine-4-carboxaldehyde for tert-butyl 4-oxopiperidine-1-carboxylate. LCMS calc. for C₂₀H₃₂ClN₆O₂[M+H]⁺: m/z=423.2; Found: 423.2.

### Step 3: tert-butyl 4-((6S,6aS)-2-(2-(Methoxymethoxy)phenyl)-6-methyl-5,6,6a,7,9,10-hexahydro-8H-pyrazino[1′,2′:4,5]pyrazino[2,3-c]pyridazin-8-yl)piperidine-1-carboxylate

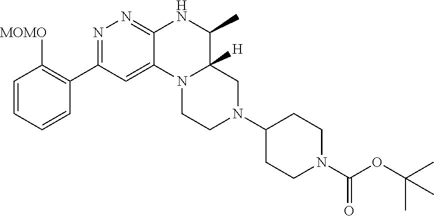

The title compound was synthesized by procedures analogous to those outlined in Example 3, Step 2 substituting tert-butyl (S)-4-((2-chloro-5,6,6a,7,9,10-hexahydro-8H-pyrazino[1′,2′:4,5]pyrazino[2,3-c]pyridazin-8-yl)methyl)piperidine-1-carboxylate for (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide. LCMS calc. for C₂₈H₄₁N₆O₄ [M+H]⁺: m/z=525.3; Found: 525.5.

### Step 4: (6S,6aS)-2-(2-(Methoxymethoxy)phenyl)-6-methyl-8-(piperidin-4-yl)-6,6a,7,8,9,10-hexahydro-5H-pyrazino[1′,2′:4,5]pyrazino[2,3-c]pyridazine

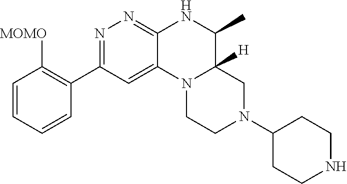

The title compound was synthesized by procedures analogous to those outlined in Example 3, Step 3 substituting tert-butyl (S)-4-((2-(2-(methoxymethoxy)phenyl)-5,6,6a,7,9,10-hexahydro-8H-pyrazino[1′,2′:4,5]pyrazino[2,3-c]pyridazin-8-yl)methyl)piperidine-1-carboxylate for tert-butyl 4-((6S,6aS)-2-(2-(Methoxymethoxy)phenyl)-6-methyl-5,6,6a,7,9,10-hexahydro-8H-pyrazino[1′,2′:4,5]pyrazino[2,3-c]pyridazin-8-yl)piperidine-1-carboxylate. LCMS calc. for C₂₃H₃₃N₆O₂ [M+H]⁺: m/z=425.3; Found: 425.4.

### Step 5: (2S,4R)-4-((tert-Butyldimethylsilyl)oxy)-1-((R)-2-(3-(2-(4-((6S,6aS)-2-(2-(methoxymethoxy)phenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

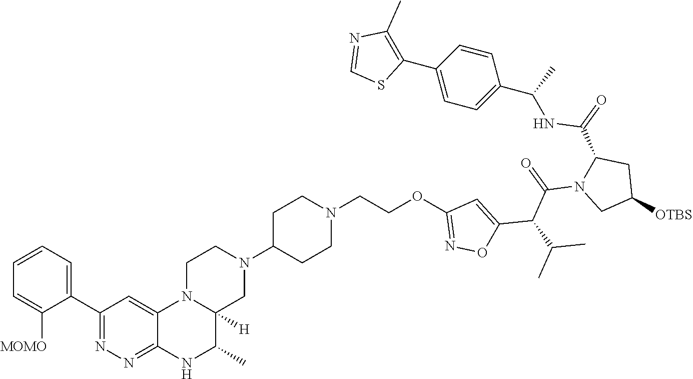

The title compound was synthesized by procedures analogous to those outlined in Example 3, Step 4 substituting (S)-2-(2-(Methoxymethoxy)phenyl)-8-(piperidin-4-ylmethyl)-6,6a,7,8,9,10-hexahydro-5H-pyrazino[1′,2′:4,5]pyrazino[2,3-c]pyridazine for (6S,6aS)-2-(2-(Methoxymethoxy)phenyl)-6-methyl-8-(piperidin-4-yl)-6,6a,7,8,9,10-hexahydro-5H-pyrazino[1′,2′:4,5]pyrazino[2,3-c]pyridazine. LCMS calc. for C₅₆H₇₉N₁₀O₇SSi [M+H]⁺: m/z=1063.6; Found: 1063.6.

### Step 6: 4—((S)-2-((S)-2-(6-(2,5-Dioxo-2,5-dihydro-1H-pyrrol-1-yl)hexanamido)-3-methylbutanamido)-5-ureidopentanamido)benzyl (6S,6aS)-8-(1-(2-((5-((R)-1-((2S,4R)-4-((tert-butyldimethylsilyl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-(methoxymethoxy)phenyl)-6-methyl-6,6a,7,8,9,10-hexahydro-5H-pyrazino[1′,2′:4,5]pyrazino[2,3-c]pyridazine-5-carboxylate

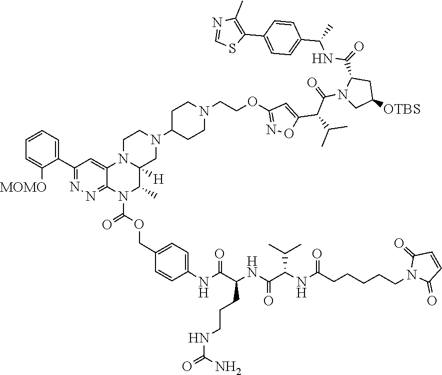

The title compound was synthesized by procedures analogous to those outlined in Example 3, Step 5 substituting (2S,4R)-4-((tert-butyldimethylsilyl)oxy)-1-((R)-2-(3-(2-(4-(((S)-2-(2-(methoxymethoxy)phenyl)-5,6,6a,7,9,10-hexahydro-8H-pyrazino[1′,2′:4,5]pyrazino[2,3-c]pyridazin-8-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide for (2S,4R)-4-((tert-Butyldimethylsilyl)oxy)-1—((R)-2-(3-(2-(4-((6S,6aS)-2-(2-(methoxymethoxy)phenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide. LCMS calc. for C₈₅H₁₁₈N₁₆O₁₅SSi [M+2H]2+: m/z=831.4; Found: 831.4.

### Step 7: 4—((S)-2-((S)-2-(6-(2,5-Dioxo-2,5-dihydro-1H-pyrrol-1-yl)hexanamido)-3-methylbutanamido)-5-ureidopentanamido)benzyl (6S,6aS)-8-(1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

The title compound was synthesized by procedures analogous to those outlined in Example 3, Step 6. LCMS calc. for C₇₇H₁₀₀N₁₆O₁₄S [M+2H]2+: m/z=752.4; Found: 752.2.

### Example 21. Synthesis of Compound 2.63—4-((S)-2-((S)-2-(3-(2-(2,5-Dioxo-2,5-dihydro-1H-pyrrol-1-yl)ethoxy)propanamido)-3-methylbutanamido)-5-ureidopentanamido)benzyl (6S,6aS)-2-(2-hydroxyphenyl)-6-methyl-8-(1-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperidin-4-yl)-6,6a,7,8,9,10-hexahydro-5H-pyrazino[1′,2′:4,5]pyrazino[2,3-c]pyridazine-5-carboxylate

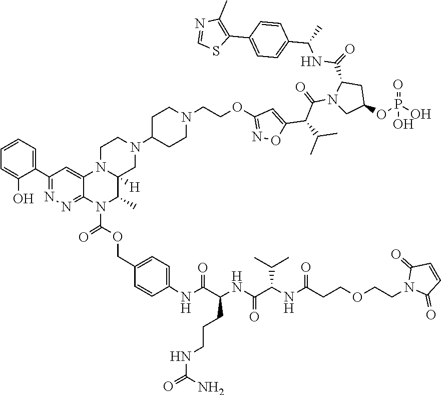 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)[C@@H](NC(=O)CCOCCN7C(=O)C=CC7=O)C(C)C)cc6)[C@@H](C)[C@@H]5C4)CC3)no2)C(C)C)cc1]

### Step 1: 2-((5-((R)-1-((2S,4R)-4-Hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl acetate

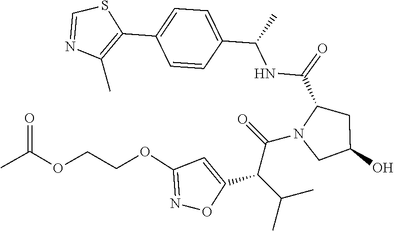 [SMILES: CC(=O)OCCOc1cc([C@H](C(=O)N2C[C@H](O)C[C@H]2C(=O)N[C@@H](C)c2ccc(-c3scnc3C)cc2)C(C)C)on1]

To a solution of (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide (500 mg, 0.761 mmol, Intermediate 2) and TEA (0.318 mL, 2.28 mmol) in DCM (10.0 mL) was added acetyl chloride (0.135 mL, 1.90 mmol). The resulting mixture was stirred at rt for 24 h. The reaction mixture was diluted with water (10 mL), the two phases were separated, and the aqueous phase was extracted with DCM (3×5.0 mL). The combined organic layers were dried over sodium sulfate, filtered, and concentrated. The crude residue was dissolved in THE (5.0 mL) then TBAF solution (0.761 mL, 0.791 mmol, 1M in THF) was added. The reaction was stirred at rt for 1 h. The reaction mixture was concentrated and purified by silica gel chromatography (0-7% MeOH/DCM) to afford the title compound (350 mg, 0.599 mmol, 78.6% yield) as a colorless solid. LCMS calc. for C₂₉H₃₇N₄O₇S [M+H]⁺: m/z=585.2; Found: 585.1.

### Step 2: 2-((5-((R)-1-((2S,4R)-4-((Di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl acetate

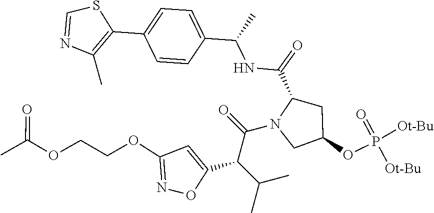 [SMILES: CC(=O)OCCOc1cc([C@H](C(=O)N2C[C@H](OP(=O)(OC(C)(C)C)OC(C)(C)C)C[C@H]2C(=O)N[C@@H](C)c2ccc(-c3scnc3C)cc2)C(C)C)on1]

To a solution of 2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl- I-oxobutan-2-yl)isoxazol-3-yl)oxy)ethyl acetate (258 mg, 0.441 mmol) and 1H-tetrazole (4.90 mL, 2.21 mmol, 0.45 M in MeCN) in THE (5.0 mL) was added di-tert-butyl diisopropylphosphoramidite (0.557 mL, 1.77 mmol). The reaction was stirred at rt for 24 h before addition of more 1H-tetrazole (4.90 mL, 2.21 mmol, 0.45 M in MeCN) and di-tert-butyl diisopropylphosphoramidite (0.557 mL, 1.77 mmol). The reaction was stirred a further 8 h before addition of more 1H-tetrazole (4.90 mL, 2.21 mmol, 0.45 M in MeCN) and di-tert-butyl diisopropylphosphoramidite (0.557 mL, 1.77 mmol). The reaction mixture was stirred for 16 h then concentrated before being partitioned between sat. NaHCO₃ (aq.) (10.0 mL) and DCM (10.0 mL). The two phases were separated and the aqueous layer was extracted with DCM (3×10 mL). The combined organic layers were dried over sodium sulfate, filtered, and concentrated. The crude residue was used directly in the next step without further purification. LCMS calc. for C₃₇H₅₄N₄O₁₀PS [M+H]⁺: m/z=777.3; Found: 777.4.

### Step 3: Di-tert-butyl ((3R,5S)-1-((R)-2-(3-(2-hydroxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate

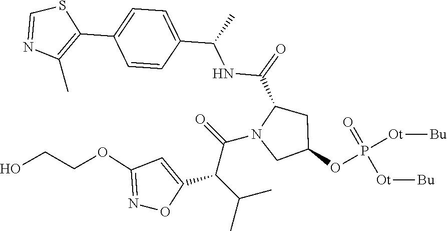

To a solution 2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl acetate (343 mg, 0.441 mmol) in MeOH (3.0 mL) was added potassium carbonate (183 mg, 1.32 mol) at 0° C. The reaction mixture was warmed to rt and stirred for 5 min. The mixture was filtered before being diluted with sat. NH₄Cl (aq.) (10 mL) and DCM (10 mL). The two phases were separated and the aqueous layer was extracted with DCM (3×10 mL). The combined organic layers were dried over sodium sulfate, filtered, and concentrated under reduced pressure. The crude residue was purified by silica gel chromatography (0-8% MeOH/DCM) to afford the title compound (200 mg, 0.272 mmol) as a yellow solid. LCMS calc. for C₃₅H₅₂N₄O₉PS [M+H]⁺: m/z=735.3; Found: 735.5.

### Step 4: 4—((S)-2-((S)-2-((((9H-Fluoren-9-yl)methoxy)carbonyl)amino)-3-methylbutanamido)-5-ureidopentanamido)benzyl (6S,6aS)-2-(2-hydroxyphenyl)-6-methyl-8-(piperidin-4-yl)-6,6a,7,8,9,10-hexahydro-5H-pyrazino[1′,2′:4,5]pyrazino[2,3-c]pyridazine-5-carboxylate

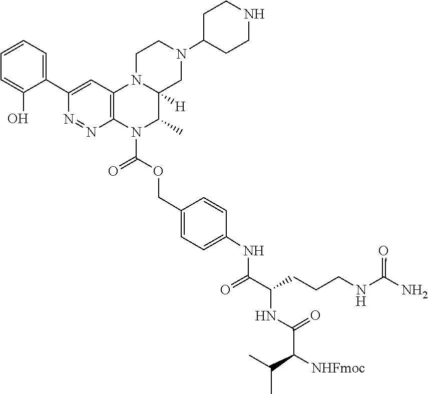

To a solution of tert-butyl 4-((6S,6aS)-2-(2-(methoxymethoxy)phenyl)-6-methyl-5,6,6a,7,9,10-hexahydro-8H-pyrazino[1′,2′:4,5]pyrazino[2,3-c]pyridazin-8-yl)piperidine-1-carboxylate (100 mg, 0.191 mmol, Example 20, Step 3) and Fmoc-Val-Cit-PAB-PNP (146 mg, 0.191 mmol, CAS #: 863971-53-3) in 1,4-dioxane (3.0 mL) and DMSO (0.15 mL) was added 2,6-lutidine (0.333 mL, 2.86 mmol). The resulting mixture was heated to 60° C. for 3 days. The mixture was cooled to rt, poured into water (50 mL), and the solid was collected by filtration. The resulting solid was washed with water and dried before being dissolved in a solution of conc. HCl (aq.) and IPA (1:1 v/v) (2.5 mL). The mixture was stirred at rt for 15 min. The solution was diluted with water and purified by prep-HPLC on a C18 column (5-40% MeCN/0.1% TFA (aq.)) to afford the title compound as a TFA salt (51.6 mg, 41.7 μmol, 21.9% yield) as a yellow solid. LCMS calc. for C₅₅H₆₆N₁₁O₈[M+H]⁺: m/z=1008.5; Found: 1008.4.

### Step 5: 4—((S)-2-((S)-2-((((9H-Fluoren-9-yl)methoxy)carbonyl)amino)-3-methylbutanamido)-5-ureidopentanamido)benzyl (6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

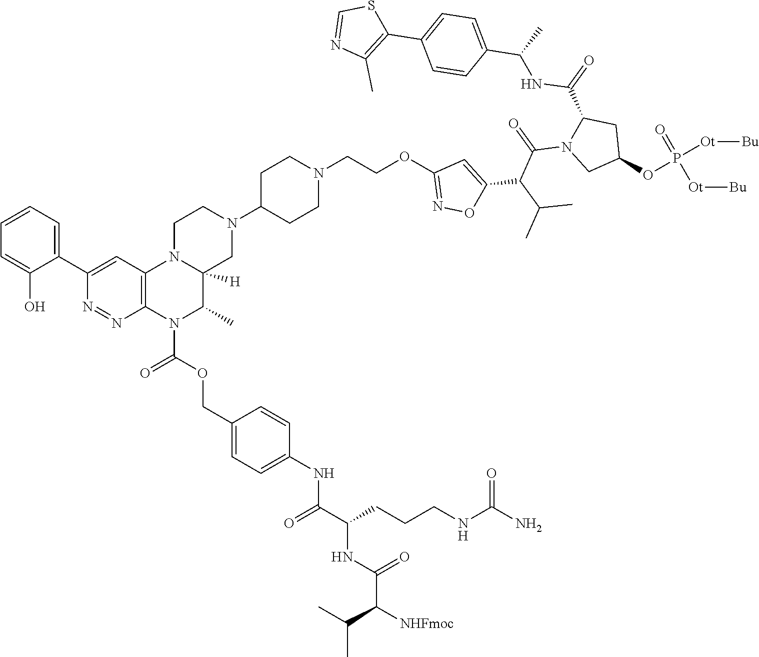

The title compound was synthesized by procedures analogous to those outlined in Example 3, Step 4 using 4-((S)-2-((S)-2-((((9H-fluoren-9-yl)methoxy)carbonyl)amino)-3-methylbutanamido)-5-ureidopentanamido)benzyl (6S,6aS)-2-(2-hydroxyphenyl)-6-methyl-8-(piperidin-4-yl)-6,6a,7,8,9,10-hexahydro-5H-pyrazino[1′,2′:4,5]pyrazino[2,3-c]pyridazine-5-carboxylate and di-tert-butyl ((3R,5S)-1-((R)-2-(3-(2-hydroxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate instead of (S)-2-(2-(methoxymethoxy)phenyl)-8-(piperidin-4-ylmethyl)-6,6a,7,8,9,10-hexahydro-5H-pyrazino[1′,2′:4,5]pyrazino[2,3-c]pyridazine and (2S,4R)-4-hydroxy-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide. LCMS calc. for C₉₀H₁₁₆N₁₅O₁₆PS [M+2H]2+: m/z=862.9; Found: 863.2.

### Step 6: 4—((S)-2-((S)-2-Amino-3-methylbutanamido)-5-ureidopentanamido)benzyl (6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

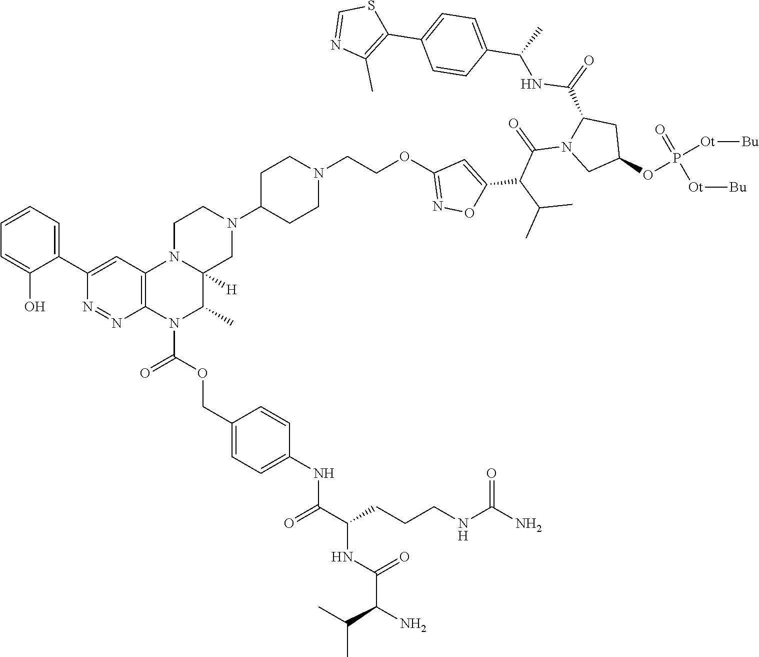

The title compound was synthesized by procedures analogous to those outlined in Example 11, Step 4 in addition to isolation by pouring the reaction mixture into ice cold 1N HCl solution (aq.) prior to purification using prep-HPLC on a C18 column (10-40% MeCN/0.1% TFA (aq.)). LCMS calc. for C₇₅H₁₀₆N₁₅O₁₄PS [M+2H]2+: m/z=751.9; Found: 752.3.

### Step 7: 4—((S)-2-((S)-2-(3-(2-(2,5-Dioxo-2,5-dihydro-1H-pyrrol-1-yl)ethoxy)propanamido)-3-methylbutanamido)-5-ureidopentanamido)benzyl (6S,6aS)-2-(2-hydroxyphenyl)-6-methyl-8-(1-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperidin-4-yl)-6,6a,7,8,9,10-hexahydro-5H-pyrazino[1′,2′:4,5]pyrazino[2,3-c]pyridazine-5-carboxylate

The title compound was synthesized by procedure analogous to those outlined in Example 5, Step 2. LCMS calc. for C₇₆H₉₉N₁₆O₁₈PS [M+2H]2+: m/z=793.3; Found: 793.1.

### Example 22. Synthesis of Compound 2.65 —(S)-4-(6-(2,5-Dioxo-2,5-dihydro-1H-pyrrol-1-yl)hexanamido)-5-((2-(((S)-1-((4-((((6S,6aS)-8-(1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carbonyl)oxy)methyl)phenyl)amino)-1-oxo-5-ureidopentan-2-yl)amino)-2-oxoethyl)amino)-5-oxopentanoic acid

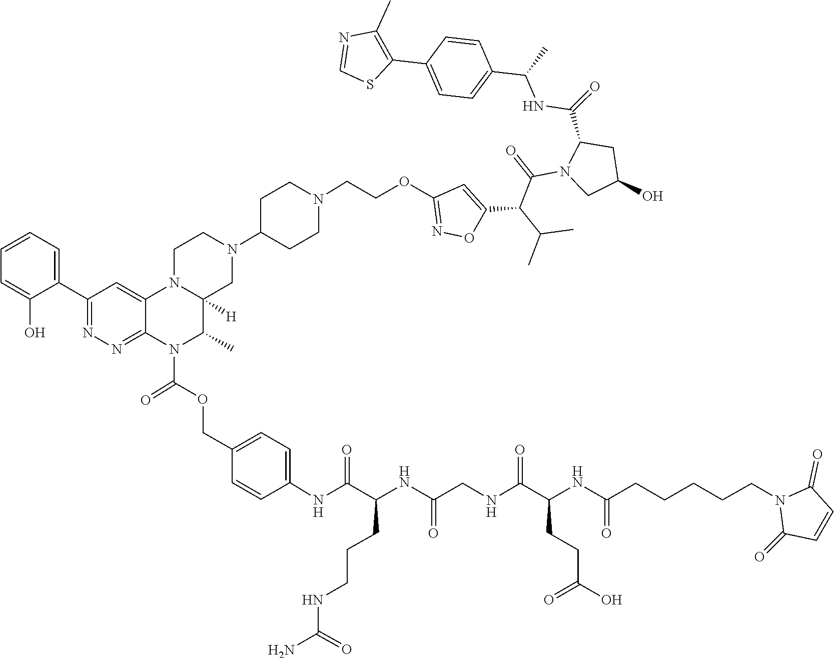 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)CNC(=O)[C@H](CCC(=O)O)NC(=O)CCCCCN7C(=O)C=CC7=O)cc6)[C@@H](C)[C@@H]5C4)CC3)no2)C(C)C)cc1]

### Step 1: (S)-2-(2-((((9H-Fluoren-9-yl)methoxy)carbonyl)amino)acetamido)-5-ureidopentanoic acid

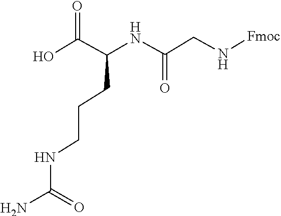

A solution of 2,5-dioxopyrrolidin-1-yl (((9H-fluoren-9-yl)methoxy)carbonyl)glycinate (968 mg, 2.45 mmol) in DME (10 mL) was added to a solution of (S)-2-amino-5-ureidopentanoic acid (430 mg, 2.45 mmol) and NaHCO₃ (227 mg, 2.70 mmol) in water (10 mL) and THE (6.5 mL) at 0° C. The solution was allowed to warm to rt and was stirred for 16 h. The reaction mixture was diluted with MTBE (20 mL) and the pH adjusted to -2 using 1N HCl (aq.). The two phases were separated and the aqueous layer was extracted with chloroform/IPA (3:1) (v/v) (6×20 mL). The combined organic layers were dried over sodium sulfate, filtered, and concentrated to afford the title compound (1.10 g, 2.42 mmol, 98.6% yield) as a sticky solid. LCMS calc. for C₂₃H₂₇N₄O₆ [M+H]⁺m/z=455.2; Found: 455.3.

### Step 2: (9H-Fluoren-9-yl)methyl (S)-(2-((1-((4-(hydroxymethyl)phenyl)amino)-1-oxo-5-ureidopentan-2-yl)amino)-2-oxoethyl)carbamate

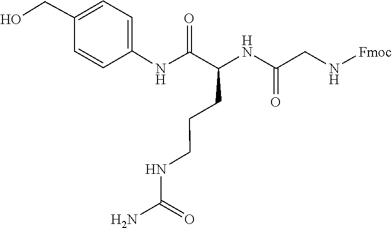

To a solution of (S)-2-(2-((((9H-fluoren-9-yl)methoxy)carbonyl)amino)acetamido)-5-ureidopentanoic acid (518 mg, 1.14 mmol), 1-hydroxybenzotriazole hydrate (145 mg, 1.14 mmol), and 1-ethyl-3-(3-dimethylaminopropyl)carbodiimide hydrochloride (328 mg, 1.71 mmol) in DMF (7.0 mL) was added (4-aminophenyl)methanol (168 mg, 1.37 mmol). The reaction mixture was stirred at rt overnight. The mixture was diluted with water and DMF before being purified by prep-HPLC on a C18 column (10-40% MeCN/0.05% TFA (aq.)) to afford the title compound (320 mg, 0.475 mmol, 47.3% yield) as a TFA salt. LCMS calc. for C₃₀H₃₄N₅O₆ [M+H]⁺m/z=560.2; Found: 560.4.

### Step 3: (S)-2-(2-Aminoacetamido)-N-(4-(hydroxymethyl)phenyl)-5-ureidopentanamide

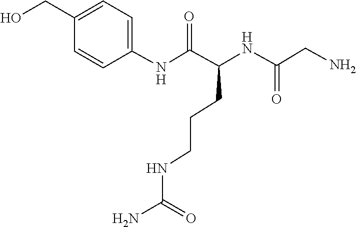 [SMILES: NCC(=O)N[C@@H](CCCNC(N)=O)C(=O)Nc1ccc(CO)cc1]

To a solution of (9H-fluoren-9-yl)methyl (S)-(2-((1-((4-(hydroxymethyl)phenyl)amino)-1-oxo-5-ureidopentan-2-yl)amino)-2-oxoethyl)carbamate (2.00 g, 3.57 mmol) in DMF (8.0 mL) was added diethylamine (0.555 mL, 5.36 mmol) at rt. The reaction mixture was stirred at rt for 40 min before being diluted with water (20 mL) and EtOAC (20 mL). The two phases were separated and the aqueous layer was extracted with EtOAc (2×20 mL). The combined organic layers were concentrated to afford the title compound (1.20 g, 3.56 mmol, 99.5% yield) as a dark red oil. LCMS calc. for C₁₅H₂₄N₅O₄ [M+H]⁺m/z=338.2; Found: 338.7.

### Step 4: tert-Butyl (S)-4-((((9H-fluoren-9-yl)methoxy)carbonyl)amino)-5-((2-(((S)-1-((4-(hydroxymethyl)phenyl)amino)-1-oxo-5-ureidopentan-2-yl)amino)-2-oxoethyl)amino)-5-oxopentanoate

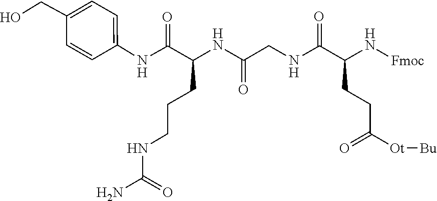

To a solution of (S)-2-(2-aminoacetamido)-N-(4-(hydroxymethyl)phenyl)-5-ureidopentanamide (1.20 g, 3.56 mmol) and NaHCO₃ (359 mg, 4.27 mmol) in DMF (8.0 mL) and water (10 mL) was added 5-(tert-butyl) 1-(2,5-dioxopyrrolidin-1-yl) (((9H-fluoren-9-yl)methoxy)carbonyl)-L-glutamate (2.04 g, 3.91 mmol) at 0° C. The reaction mixture was allowed to warm to rt and was stirred overnight. The mixture was diluted with chloroform/IPA (3:1) (v/v) (20 mL) and the resulting two phases were separated. The aqueous layer was further extracted with chloroform/IPA (3:1) (v/v) (3×20 mL). The combined organic layers were dried over sodium sulfate, filtered, and concentrated. The crude residue was purified by pre-HPLC on a C18 column (15-60% MeCN/0.05% TFA (aq.)) to afford the title compound (660 mg, 0.771 mmol, 21.7% yield) as a TFA salt. LCMS calc. for C₃₉H₄₉N₆O₉ [M+H]⁺m/z=745.4; Found: 745.2.

### Step 5: tert-Butyl (S)-4-((((9H-fluoren-9-yl)methoxy)carbonyl)amino)-5-((2-(((S)-1-((4-((((4-nitrophenoxy)carbonyl)oxy)methyl)phenyl)amino)-1-oxo-5-ureidopentan-2-yl)amino)-2-oxoethyl)amino)-5-oxopentanoate

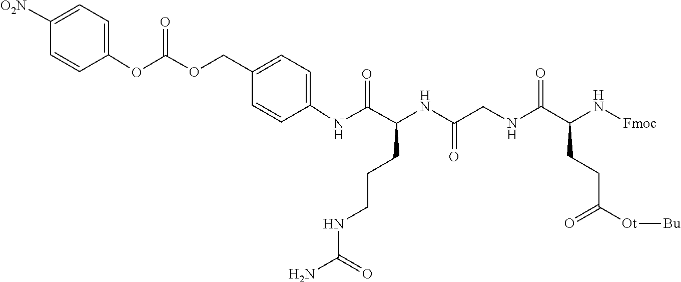

To a solution of tert-butyl (S)-4-((((9H-fluoren-9-yl)methoxy)carbonyl)amino)-5-((2-(((S)-1-((4-(hydroxymethyl)phenyl)amino)-1-oxo-5-ureidopentan-2-yl)amino)-2-oxoethyl)amino)-5-oxopentanoate (220 mg, 0.295 mmol) and bis(4-nitrophenyl) carbonate (180 mg, 0.591 mmol) in DMF (2.0 mL) was added DIPEA (82.3 μL, 0.473 mmol) at rt. The reaction mixture was stirred at rt for 3 h. The mixture was poured in MTBE (30 mL). The resulting solid was collected, washed with MTBE, and dried to afford the title compound (185 mg, 0.203 mmol, 68.8% yield) as a dark solid. LCMS calc. for C₄₆H₅₂N₇O₁₃ [M+H]⁺m/z=910.4; Found: 910.7.

### Step 6: (S)-4-((((9H-Fluoren-9-yl)methoxy)carbonyl)amino)-5-((2-(((S)-1-((4-((((6S,6aS)-2-(2-hydroxyphenyl)-6-methyl-8-(piperidin-4-yl)-6,6a,7,8,9,10-hexahydro-5H-pyrazino[1′,2′:4,5]pyrazino[2,3-c]pyridazine-5-carbonyl)oxy)methyl)phenyl)amino)-1-oxo-5-ureidopentan-2-yl)amino)-2-oxoethyl)amino)-5-oxopentanoic acid

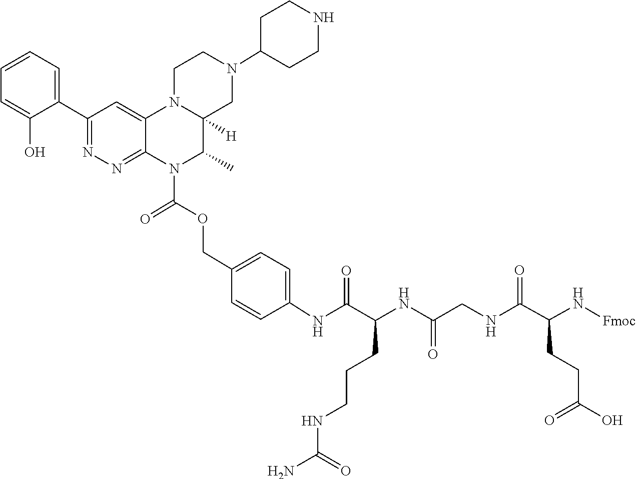

The title compound was synthesized by procedures analogous to those outlined in Example 21, Step 4 using tert-butyl (S)-4-((((9H-fluoren-9-yl)methoxy)carbonyl)amino)-5-((2-(((S)-1-((4-((((4-nitrophenoxy)carbonyl)oxy)methyl)phenyl)amino)-1-oxo-5-ureidopentan-2-yl)amino)-2-oxoethyl)amino)-5-oxopentanoate instead of Fmoc-Val-Cit-PAB-PNP (CAS #: 863971-53-3). LCMS calc. for C₅₇H₆₇N₁₂O₁₁ [M+H]⁺: m/z=1095.5; Found: 1095.4.

### Step 7: (S)-4-((((9H-Fluoren-9-yl)methoxy)carbonyl)amino)-5-((2-(((S)-1-((4-((((6S,6aS)-8-(1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carbonyl)oxy)methyl)phenyl)amino)-1-oxo-5-ureidopentan-2-yl)amino)-2-oxoethyl)amino)-5-oxopentanoic acid

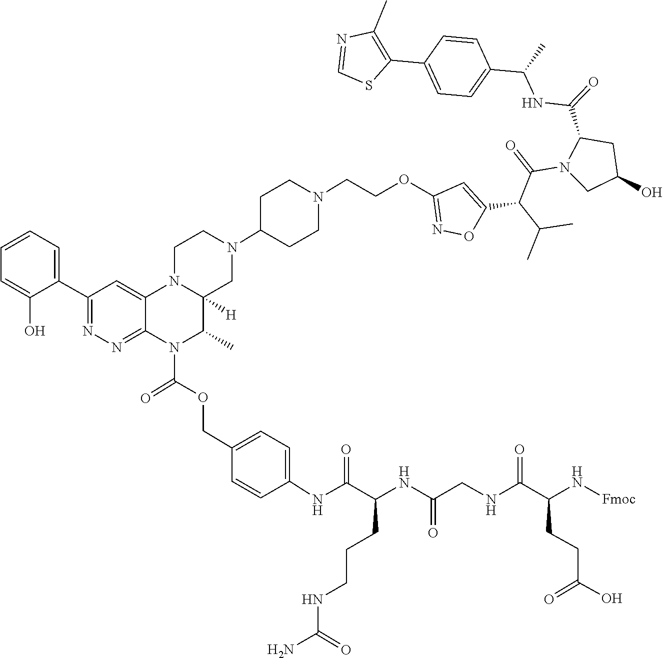

The title compound was synthesized by procedure analogous to those outlined in Example 3, Step 4 using TEA instead of NaHCO₃. LCMS calc. for C₈₄H₁₀₀N₁₆O₁₆S [M+2H]²⁺. m/z=810.4; Found: 810.3.

### Step 8: (S)-4-Amino-5-((2-(((S)-1-((4-((((6S,6aS)-8-(1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carbonyl)oxy)methyl)phenyl)amino)-1-oxo-5-ureidopentan-2-yl)amino)-2-oxoethyl)amino)-5-oxopentanoic acid

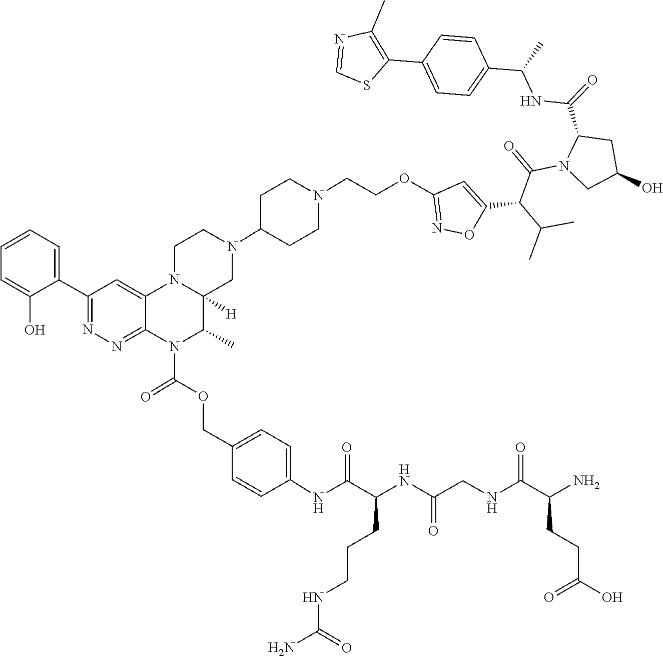 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N(C(=O)OCc6ccc(NC(=O)[C@H](CCCNC(N)=O)NC(=O)CNC(=O)[C@@H](N)CCC(=O)O)cc6)[C@@H](C)[C@@H]5C4)CC3)no2)C(C)C)cc1]

The title compound was synthesized by procedures analogous to those outlined in Example 11, Step 4. LCMS calc. for C₆₉H₉₀N₁₆O₁₄S [M+2H]²⁺: m/z=699.3; Found: 699.4.

### Step 9: (S)-4-(6-(2,5-Dioxo-2,5-dihydro-1H-pyrrol-1-yl)hexanamido)-5-((2-(((S)-1-((4-((((6S,6aS)-8-(1-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carbonyl)oxy)methyl)phenyl)amino)-1-oxo-5-ureidopentan-2-yl)amino)-2-oxoethyl)amino)-5-oxopentanoic acid

The title compound was synthesized by procedures analogous to those outlined in Example 5, Step 2. LCMS calc. for C₇₉H₁₀₁N₁₇O₁₇S [M+2H]²⁺: m/z=795.9; Found: 796.4.

### Example 23. Synthesis of Compound 2.69-4-((32S,35S)-1-(2,5-Dioxo-2,5-dihydro-1H-pyrrol-1-yl)-32-isopropyl-35-methyl-2,30,33-trioxo-6,9,12,15,18,21,24,27-octaoxa-3,31,34-triazahexatriacontan-36-amido)benzyl (6S,6aS)-2-(2-hydroxyphenyl)-6-methyl-8-(1-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperidin-4-yl)-6,6a,7,8,9,10-hexahydro-5H-pyrazino[1′,2′:4,5]pyrazino[2,3-c]pyridazine-5-carboxylate

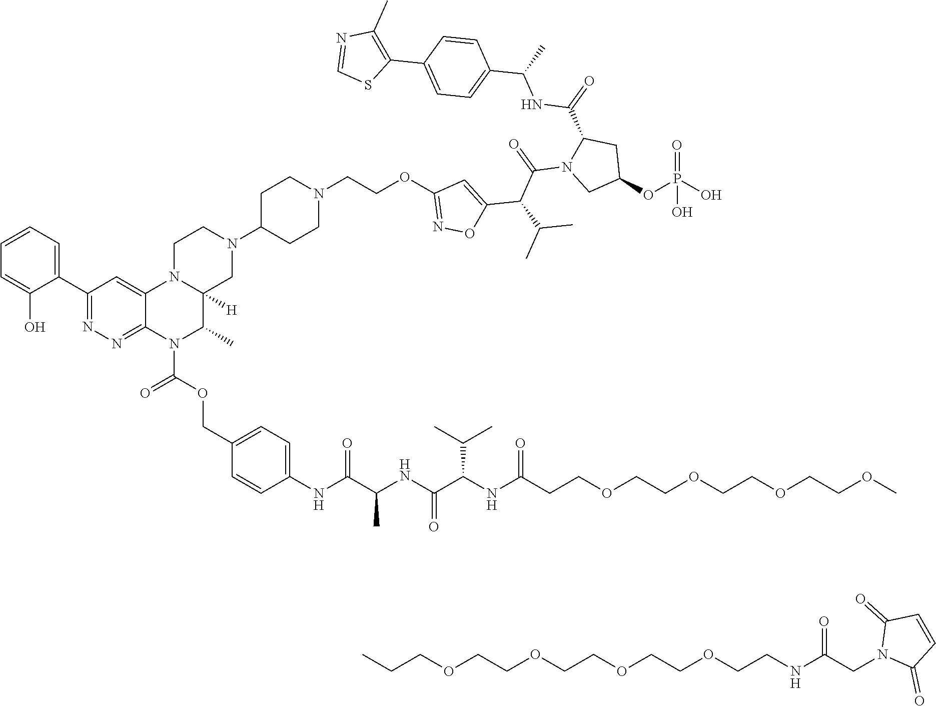

### Step 1: 4—((S)-2-((S)-2-((((9H-Fluoren-9-yl)methoxy)carbonyl)amino)-3 methylbutan amido)propanamido)benzyl (6S,6aS)-8-(1-(tert-butoxycarbonyl)piperidin-4-yl)-2-(2-(methoxymethoxy)phenyl)-6-methyl-6,6a,7,8,9,10-hexahydro-5H-pyrazino [1′,2′:4,5]pyrazino[2,3-c]pyridazine-5-carboxylate

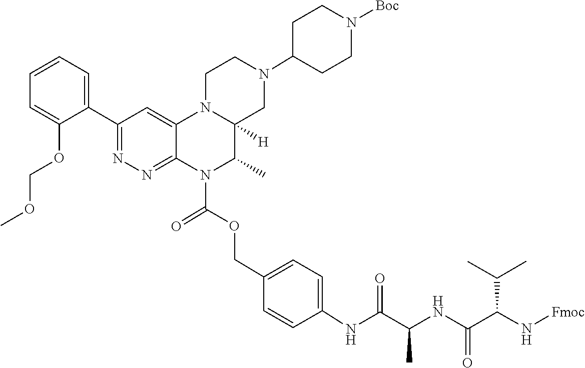

The title compound was synthesized by procedures analogous to those outlined in Example 21, Step 4 using Fmoc-Val-Ala-PAB-PNP (CAS #: 1394238-92-6) and instead of Fmoc-Val-Cit-PAB-PNP (CAS #: 863971-53-3). LCMS calc. for C₅₉H₇₂N₉O₁₀ [M+H]⁺m/z=1066.5; Found: 1067.2.

### Step 2: 4—((S)-2-((S)-2-((((9H-Fluoren-9-yl)methoxy)carbonyl)amino)-3-methylbutanamido)propanamido)benzyl (6S,6aS)-2-(2-hydroxyphenyl)-6-methyl-8-(piperidin-4-yl)-6,6a,7,8,9,10-hexahydro-5H-pyrazino[1′,2′:4,5]pyrazino[2,3-c]pyridazine-5-carboxylate

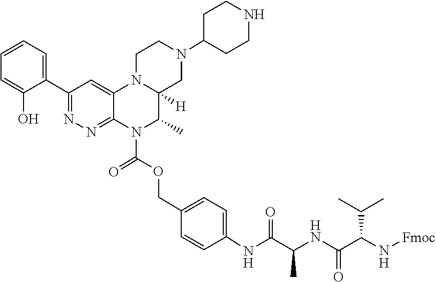

To a solution of 4-((S)-2-((S)-2-((((9H-fluoren-9-yl)methoxy)carbonyl)amino)-3 methylbutan amido)propanamido)benzyl (6S,6aS)-8-(1-(tert-butoxycarbonyl)piperidin-4-yl)-2-(2-(methoxymethoxy)phenyl)-6-methyl-6,6a,7,8,9,10-hexahydro-5H-pyrazino [1′,2′:4,5]pyrazino[2,3-c]pyridazine-5-carboxylate (140 mg, 0.13 mmol) in IPA (1.0 mL) was added conc. HCl (1.0 mL) at 0° C. The mixture was stirred at rt for 30 min. The mixture was diluted with water (1.0 mL) and purified by prep-HPLC on C18 column (20-60% MeCN/0.05% TFA (aq.)) to afford the TFA salt of the title compound (81 mg, 78 μmol, 60% yield) as a white solid.

LCMS calc. for C₅₂H₆₀N₉O₇[M+H]⁺m/z=922.5; Found: 922.3.

### Step 3: 4—((S)-2-((S)-2-((((9H-Fluoren-9-yl)methoxy)carbonyl)amino)-3-methylbutanamido)propanamido)benzyl (6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

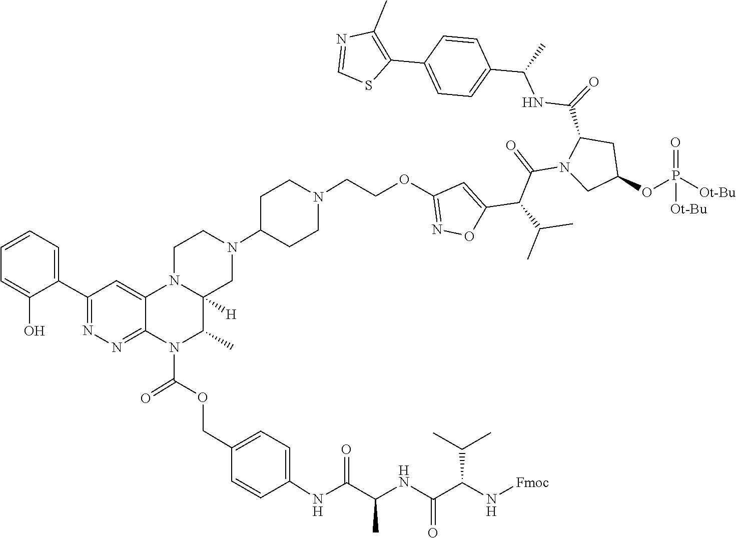

The title compound was synthesized by procedures analogous to those outlined in Example 3, Step 4 using 4-((S)-2-((S)-2-((((9H-fluoren-9-yl)methoxy)carbonyl)amino)-3-methylbutanamido)propanamido)benzyl (6S,6aS)-2-(2-hydroxyphenyl)-6-methyl-8-(piperidin-4-yl)-6,6a,7,8,9,10-hexahydro-5H-pyrazino[1′,2′:4,5]pyrazino[2,3-c]pyridazine-5-carboxylate and di-tert-butyl ((3R,5S)-1-((R)-2-(3-(2-hydroxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate instead of (S)-2-(2-(methoxymethoxy)phenyl)-8-(piperidin-4-ylmethyl)-6,6a,7,8,9,10-hexahydro-5H-pyrazino[1′,2′:4,5]pyrazino[2,3-c]pyridazine and (2S,4R)-4-((tert-butyldimethylsilyl)oxy)-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide. LCMS calc. for C₈₇H₁₀₉N₁₃O₁₅PS [M+H]⁺m/z=1638.8; Found: 1638.7.

### Step 4: 4—((S)-2-((S)-2-Amino-3-methylbutanamido)propanamido)benzyl (6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

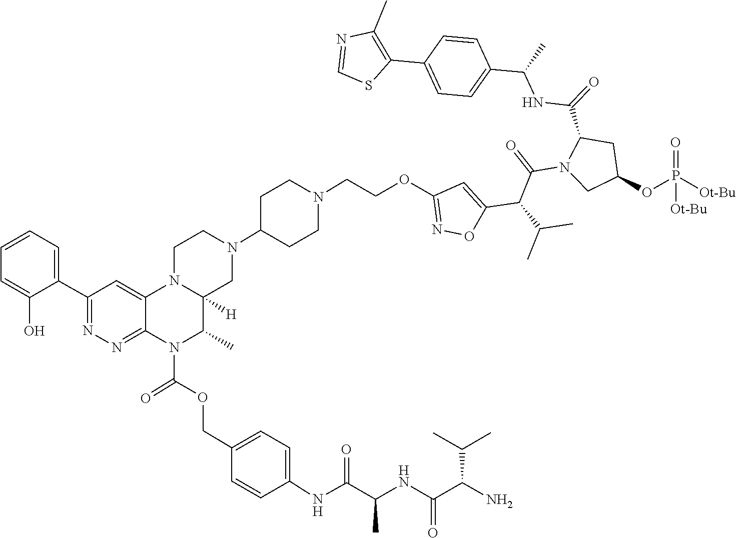 [SMILES: Cc1ncsc1-c1ccc([C@H](C)NC(=O)[C@@H]2C[C@@H](OP(=O)(OC(C)(C)C)OC(C)(C)C)CN2C(=O)[C@@H](c2cc(OCCN3CCC(N4CCN5c6cc(-c7ccccc7O)nnc6N(C(=O)OCc6ccc(NC(=O)[C@H](C)NC(=O)[C@@H](N)C(C)C)cc6)[C@@H](C)[C@@H]5C4)CC3)no2)C(C)C)cc1]

The title compound was synthesized by procedures analogous to those outlined in Example 11, Step 4. LCMS calc. for C₇₂H₉₉N₁₃O₁₃PS [M+H]⁺m/z=1416.7; Found: 1416.5.

### Step 5: 4-((34S,37S)-34-Isopropyl-2,2,37-trimethyl-4,32,35-trioxo-3,8,11,14,17,20,23,26,29-nonaoxa-5,33,36-triazaoctatriacontan-38-amido)benzyl (6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate

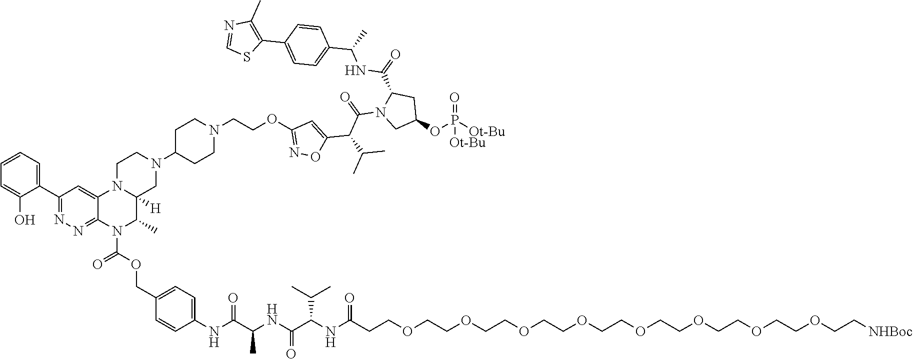 [SMILES: Cc1ncsc1-c1ccc([C@H](C)NC(=O)[C@@H]2C[C@@H](OP(=O)(OC(C)(C)C)OC(C)(C)C)CN2C(=O)[C@@H](c2cc(OCCN3CCC(N4CCN5c6cc(-c7ccccc7O)nnc6N(C(=O)OCc6ccc(NC(=O)[C@H](C)NC(=O)[C@@H](NC(=O)CCOCCOCCOCCOCCOCCOCCOCCOCCNC(=O)OC(C)(C)C)C(C)C)cc6)[C@@H](C)[C@@H]5C4)CC3)no2)C(C)C)cc1]

The title compound was synthesized by procedures analogous those outlined in Example 22, Step 2 using 4-((S)-2-((S)-2-amino-3-methylbutanamido)propanamido)benzyl (6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-2-(2-hydroxyphenyl)-6-methyl-6,6a,7,8,9,10-hexahydro-5H-pyrazino[1′,2′:4,5]pyrazino[2,3-c]pyridazine-5-carboxylate and 2,2-dimethyl-4-oxo-3,8,11,14,17,20,23,26,29-nonaoxa-5-azadotriacontan-32-oic acid instead of (S)-2-(2-((((9H-fluoren-9-yl)methoxy)carbonyl)amino)acetamido)-5-ureidopentanoic acid and (4-aminophenyl)methanol. LCMS calc. for C₉₆H₁₄₄N₁₄O₂₄PS [M+H]⁺m/z=1940.0; Found: 1940.0

### Step 6: 4-((29S,32S)-1-Amino-29-isopropyl-32-methyl-27,30-dioxo-3,6,9,12,15,18,21,24-octaoxa-28,31-diazatritriacontan-33-amido)benzyl (6S,6aS)-2-(2-hydroxyphenyl)-6-methyl-8-(1-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperidin-4-yl)-6,6a,7,8,9,10-hexahydro-5H-pyrazino[1′,2′:4,5]pyrazino[2,3-c]pyridazine-5-carboxylate

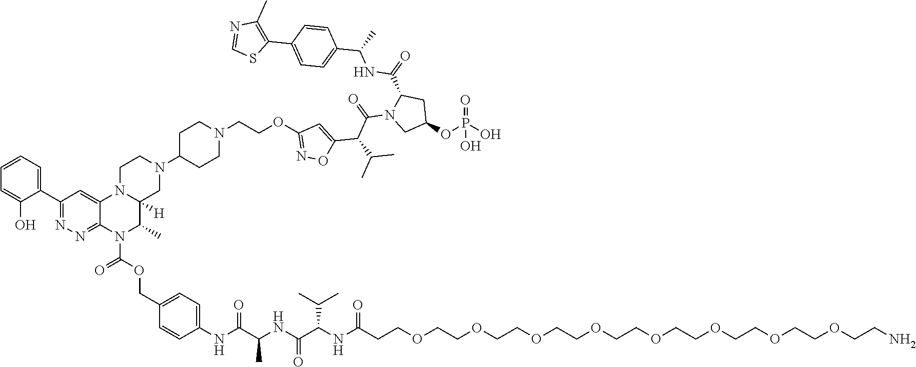 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7O)nnc6N(C(=O)OCc6ccc(NC(=O)[C@H](C)NC(=O)[C@@H](NC(=O)CCOCCOCCOCCOCCOCCOCCOCCOCCN)C(C)C)cc6)[C@@H](C)[C@@H]5C4)CC3)no2)C(C)C)cc1]

The title compound was synthesized by procedure analogous to those outlined in Example 4, Step 2. LCMS calc. for C₈₃H₁₂₀NwO₂₂PS [M+H]⁺m/z=1727.8; Found: 1727.5.

### Step 7: 4-((32S,35S)-1-(2,5-Dioxo-2,5-dihydro-1H-pyrrol-1-yl)-32-isopropyl-35-methyl-2,30,33-trioxo-6,9,12,15,18,21,24,27-octaoxa-3,31,34-triazahexatriacontan-36-amido)benzyl (6S,6aS)-2-(2-hydroxyphenyl)-6-methyl-8-(1-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperidin-4-yl)-6,6a,7,8,9,10-hexahydro-5H-pyrazino[1′,2′:4,5]pyrazino[2,3-c]pyridazine-5-carboxylate

The title compound was synthesized by procedure analogous to those outlined in Example 4, Step 3 using 2,5-dioxopyrrolidin-1-yl 2-(2,5-dioxo-2,5-dihydro-1H-pyrrol-1-yl)acetate instead of 6-maleimidohexanoic acid N-hydroxysuccinimide ester. LCMS calc. for C₈₉H₁₂₃N₁₅O₂₅PS [M+H]⁺ m/z=1864.8; Found: 1864.4.

### Example 24. Synthesis of Compound 2.70 —(3R,5S)-1-((R)-2-(3-(2-(4-((6S,6aS)-2-(2-((4-((S)-2-(1-((14-(2,5-Dioxo-2,5-dihydro-1H-pyrrol-1-yl)-3,6,9,12-tetraoxatetradecyl)carbamoyl)cyclobutane-1-carboxamido)-5-ureidopentanamido)benzyl)oxy)phenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate

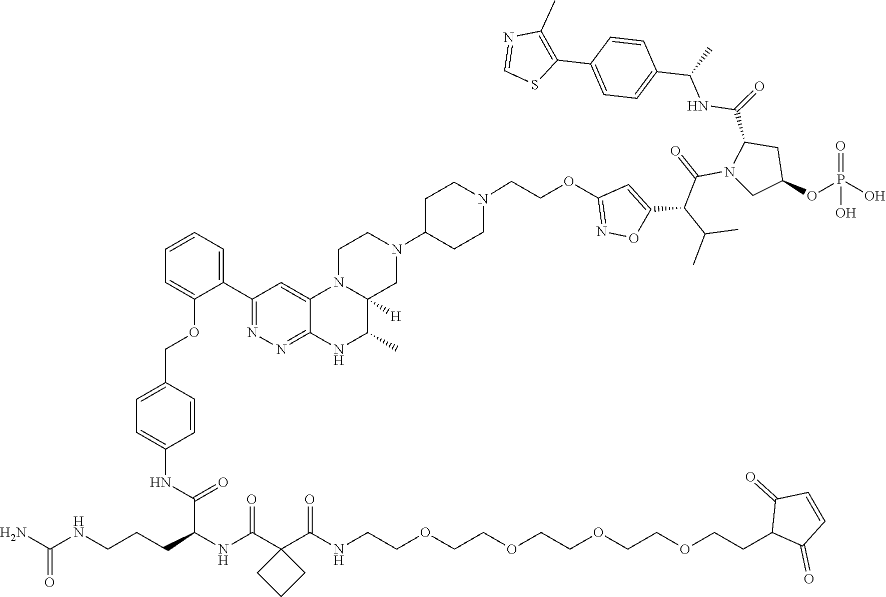 [SMILES: Cc1ncsc1-c1ccc([C@H](C)NC(=O)[C@@H]2C[C@@H](OP(=O)(O)O)CN2C(=O)[C@@H](c2cc(OCCN3CCC(N4CCN5c6cc(-c7ccccc7OCc7ccc(NC(=O)[C@H](CCCNC(N)=O)NC(=O)C8(C(=O)NCCOCCOCCOCCOCCC9C(=O)C=CC9=O)CCC8)cc7)nnc6N[C@@H](C)[C@@H]5C4)CC3)no2)C(C)C)cc1]

### Step 1: tert-Butyl 4-((6S,6aS)-2-(2-((4-((S)-2-(1-(ethoxycarbonyl)cyclobutane-1-carboxamido)-5-ureidopentanamido)benzyl)oxy)phenyl)-6-methyl-5,6,6a,7,9,10-hexahydro-8H-pyrazino[1′,2′:4,5]pyrazino[2,3-c]pyridazin-8-yl)piperidine-1-carboxylate

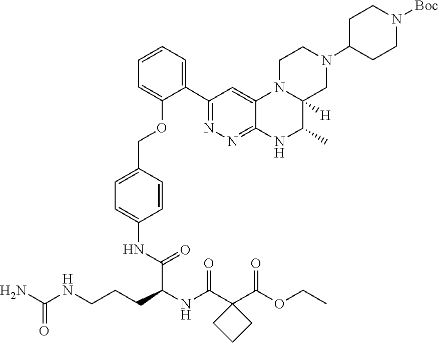 [SMILES: CCOC(=O)C1(C(=O)N[C@@H](CCCNC(N)=O)C(=O)Nc2ccc(COc3ccccc3-c3cc4c(nn3)N[C@@H](C)[C@@H]3CN(C5CCN(C(=O)OC(C)(C)C)CC5)CCN43)cc2)CCC1]

The title compound was synthesized by procedures analogous to those outlined in Example 18, Step 1 using tert-butyl 4-((6S,6aS)-2-chloro-6-methyl-5,6,6a,7,9,10-hexahydro-8H-pyrazino[1′,2′:4,5]pyrazino[2,3-c]pyridazin-8-yl)piperidine-1-carboxylate (Example 20, Step 2) instead of tert-butyl 4-[[(10S)-4-chloro-1,5,6,8,12-pentazatricyclo[8.4.0.02,7]tetradeca-2,4,6-trien-12-yl]methyl]piperidine-1-carboxylate. LCMS calc. for C₄₇H₆₅N₁₀O₈ [M+H]⁺m/z=897.5; Found: 897.3.

### Step 2: 1—(((S)-1-((4-((2-((6S,6aS)-8-(1-(tert-Butoxycarbonyl)piperidin-4-yl)-6-methyl-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid

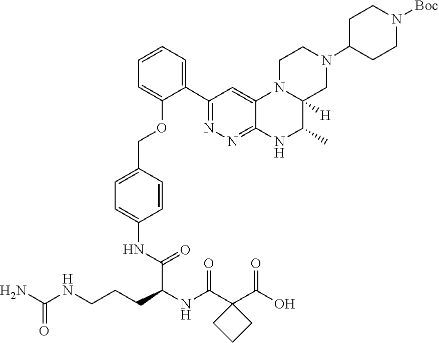 [SMILES: C[C@@H]1Nc2nnc(-c3ccccc3OCc3ccc(NC(=O)[C@H](CCCNC(N)=O)NC(=O)C4(C(=O)O)CCC4)cc3)cc2N2CCN(C3CCN(C(=O)OC(C)(C)C)CC3)C[C@@H]12]

To a solution of tert-butyl 4-((6S,6aS)-2-(2-((4-((S)-2-(1-(ethoxycarbonyl)cyclobutane-1-carboxamido)-5-ureidopentanamido)benzyl)oxy)phenyl)-6-methyl-5,6,6a,7,9,10-hexahydro-8H-pyrazino[1′,2′:4,5]pyrazino[2,3-c]pyridazin-8-yl)piperidine-1-carboxylate (65 mg, 73 μmol) in THE (1.0 mL) and water (1.0 mL) was added lithium hydroxide monohydrate (9.1 mg, 0.22 mmol). The reaction mixture was stirred at rt for 1 h. The mixture was concentrated and the crude residue was purified by prep-HPLC on C18 column (10-35% MeCN/0.05% TFA (aq.)) to afford the TFA salt of the title compound (45 mg, 51 μmol, 70% yield) as a white solid. LCMS calc. for C₄₅H₆₁N₁₀O₈ [M+H]⁺m/z=869.5; Found: 869.8.

### Step 3: 1—(((S)-1-((4-((2-((6S,6aS)-6-Methyl-8-(piperidin-4-yl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid

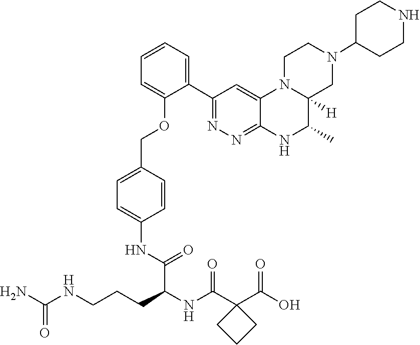 [SMILES: C[C@@H]1Nc2nnc(-c3ccccc3OCc3ccc(NC(=O)[C@H](CCCNC(N)=O)NC(=O)C4(C(=O)O)CCC4)cc3)cc2N2CCN(C3CCNCC3)C[C@@H]12]

The title compound was synthesized by procedures analogous to those outlined in Example 18, Step 2. LCMS calc. for C₄₀H₅₃N₁₀O₆[M+H]⁺m/z=769.4; Found: 769.7.

### Step 4: 1—(((S)-1-((4-((2-((6S,6aS)-8-(1-(2-((5-((R)-1-((2S,4R)-4-((Di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-6-methyl-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid

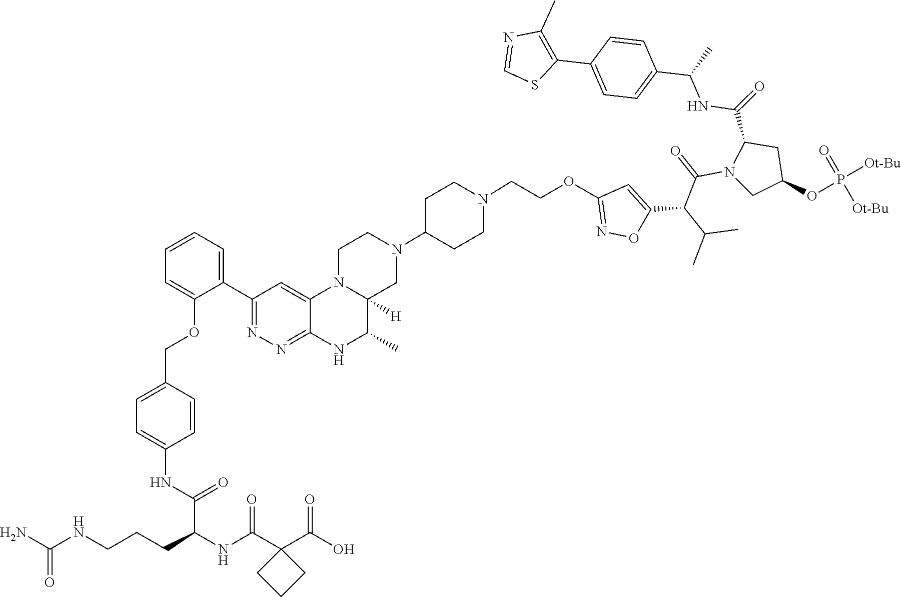 [SMILES: Cc1ncsc1-c1ccc([C@H](C)NC(=O)[C@@H]2C[C@@H](OP(=O)(OC(C)(C)C)OC(C)(C)C)CN2C(=O)[C@@H](c2cc(OCCN3CCC(N4CCN5c6cc(-c7ccccc7OCc7ccc(NC(=O)[C@H](CCCNC(N)=O)NC(=O)C8(C(=O)O)CCC8)cc7)nnc6N[C@@H](C)[C@@H]5C4)CC3)no2)C(C)C)cc1]

The title compound was synthesized by procedures analogous to those outlined in Example 3, Step 4 using 1-(((S)-1-((4-((2-((6S,6aS)-6-methyl-8-(piperidin-4-yl)-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid and di-tert-butyl ((3R,5S)-1-((R)-2-(3-(2-hydroxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate instead of (S)-2-(2-(methoxymethoxy)phenyl)-8-(piperidin-4-ylmethyl)-6,6a,7,8,9,10-hexahydro-5H-pyrazino[1′,2′:4,5]pyrazino[2,3-c]pyridazine and (2S,4R)-4-hydroxy-1-((R)-2-(3-(2-hydroxyethoxy)isoxazol-5-yl)-3-methylbutanoyl)-N—((S)-1-(4-(4-methylthiazol-5-yl)phenyl)ethyl)pyrrolidine-2-carboxamide. LCMS calc. for C₇₅H₁₀₂N₁₄O₁₄PS [M+H]⁺m/z=1485.7; Found: 1485.7.

### Step 5: Di-tert-butyl ((3R,5S)-1-((R)-2-(3-(2-(4-((6S,6aS)-2-(2-((4-((S)-2-(1-((14-(2,5-dioxo-2,5-dihydro-1H-pyrrol-1-yl)-3,6,9,12-tetraoxatetradecyl)carbamoyl)cyclobutane-1-carboxamido)-5-ureidopentanamido)benzyl)oxy)phenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate

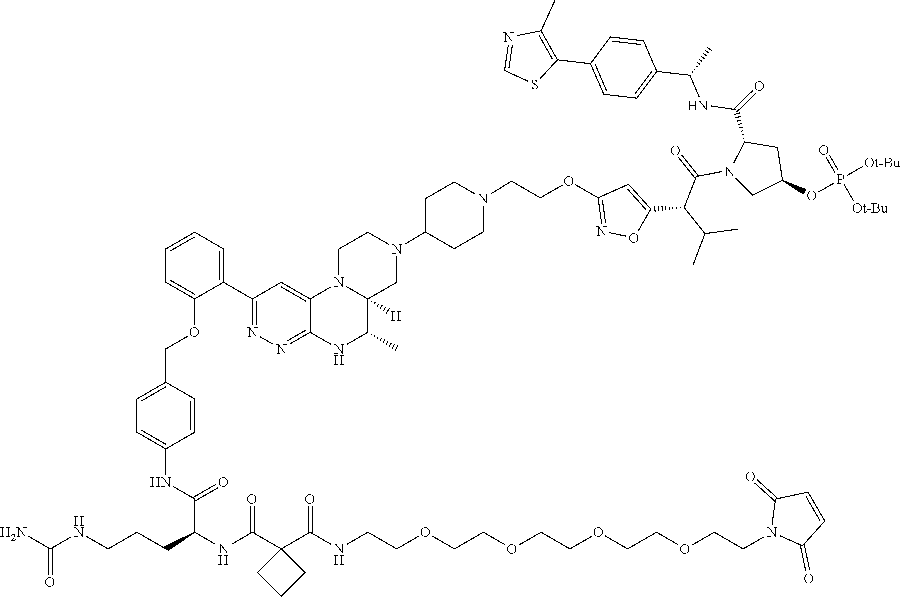 [SMILES: Cc1ncsc1-c1ccc([C@H](C)NC(=O)[C@@H]2C[C@@H](OP(=O)(OC(C)(C)C)OC(C)(C)C)CN2C(=O)[C@@H](c2cc(OCCN3CCC(N4CCN5c6cc(-c7ccccc7OCc7ccc(NC(=O)[C@H](CCCNC(N)=O)NC(=O)C8(C(=O)NCCOCCOCCOCCOCCN9C(=O)C=CC9=O)CCC8)cc7)nnc6N[C@@H](C)[C@@H]5C4)CC3)no2)C(C)C)cc1]

The title compound was synthesized by procedures analogous to those outlined in Example 22, Step 2 using 1-(((S)-1-((4-((2-((6S,6aS)-8-(1-(2-((5-((R)-1-((2S,4R)-4-((di-tert-butoxyphosphoryl)oxy)-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)piperidin-4-yl)-6-methyl-6,6a,7,8,9,10-hexahydro-5H-pyrazino[1′,2′:4,5]pyrazino[2,3-c]pyridazin-2-yl)phenoxy)methyl)phenyl)amino)-1-oxo-5-ureidopentan-2-yl)carbamoyl)cyclobutane-1-carboxylic acid and 1-[2-[2-[2-[2-(2-aminoethoxy)ethoxy]ethoxy]ethoxy]ethyl]pyrrole-2,5-dione hydrochloride instead of (S)-2-(2-((((9H-fluoren-9-yl)methoxy)carbonyl)amino)acetamido)-5-ureidopentanoic acid and (4-aminophenyl)methanol. LCMS calc. for C₈₉H₁₂₄N₁₆O₁₉PS [M+H]⁺m/z=1783.9; Found: 1783.8.

### Step 6: (3R,5S)-1-((R)-2-(3-(2-(4-((6S,6aS)-2-(2-((4-((S)-2-(1-((14-(2,5-Dioxo-2,5-dihydro-1H-pyrrol-1-yl)-3,6,9,12-tetraoxatetradecyl)carbamoyl)cyclobutane-1-carboxamido)-5-ureidopentanamido)benzyl)oxy)phenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate

A solution of 12N HCl (aq.) (100 μL) was added dropwise to a solution of di-tert-butyl ((3R,5S)-1-((R)-2-(3-(2-(4-((6S,6aS)-2-(2-((4-((S)-2-(1-((14-(2,5-dioxo-2,5-dihydro-1H-pyrrol-1-yl)-3,6,9,12-tetraoxatetradecyl)carbamoyl)cyclobutane-1-carboxamido)-5-ureidopentanamido)benzyl)oxy)phenyl)-6-methyl-5,6,6a,7,9,10-hexahydro-8H-pyrazino[1′,2′:4,5]pyrazino[2,3-c]pyridazin-8-yl)piperidin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate (8.0 mg, 4.5 μmol) in isopropyl alcohol (100 μL) at 0° C. The reaction mixture was warmed to rt and stirred for 5 min. The reaction mixture was purified directly by prep-HPLC on C18 column (5-40% MeCN/0.05% TFA (aq.)) to afford the TFA salt of the title compound (3.9 mg, 2.3 μmol, 50% yield) as a white solid. LCMS calc. for C₈₁H₁₀₈N₁₆O₁₉PS [M+H]⁺m/z=1671.7; Found: 1671.6.

### Example 25. Synthesis of Compound 2.85—4-((S)-2-((S)-2-(6-(2,5-Dioxo-2,5-dihydro-1H-pyrrol-1-yl)hexanamido)-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((phosphonooxy)methoxy)phenyl)-6a,7,8,9-tetrahydropyrrolo[1′,2′:4,5]pyrazino[2,3-c]pyridazine-5(6H)-carboxylate

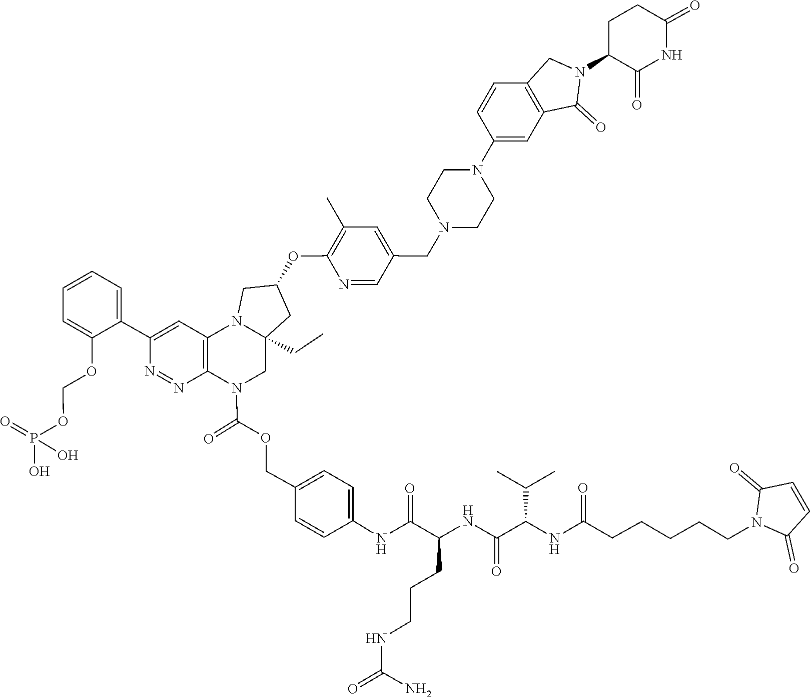 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3OCOP(=O)(O)O)nnc1N(C(=O)OCc1ccc(NC(=O)[C@H](CCCNC(N)=O)NC(=O)[C@@H](NC(=O)CCCCCN3C(=O)C=CC3=O)C(C)C)cc1)C2]

### Step 1: (2-Bromophenoxy)methyl di-tert-butyl phosphate

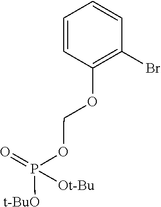 [SMILES: CC(C)(C)OP(=O)(OCOc1ccccc1Br)OC(C)(C)C]

To a solution of 2-bromophenol (4.00 g, 23.1 mmol) in DMF (50.0 mL) was added sequentially potassium carbonate (9.59 g, 69.4 mmol), sodium iodide (347 mg, 2.31 mmol), and di-tert-butyl (chloromethyl) phosphate (6.58 g, 25.4 mmol) at rt. The reaction mixture was stirred at rt over the weekend. The reaction mixture was filtered and the resulting solution was purified by prep-HPLC on a C18 column (5-45% MeCN/0.05% TFA (aq.)) to afford the TFA salt of the title compound (6.26 mg, 15.8 mmol, 68.5% yield) as a yellow oil. LCMS calc. for C₁₅H₂₅BrO₅P [M+H]⁺m/z=395.1, 397.1; Found: 395.4, 397.3.

### Step 2: 5-(1,3-Dioxolan-2-yl)-2-fluoro-3-methylpyridine

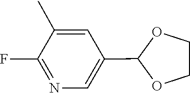

A solution of 2-fluoro-5-formyl-3-methylpyridine (2.0 g, 14 mmol), poly(ethylene glycol) (4.0 mL, 72 mmol), and pyridinium p-toluenesulfonate (360 mg, 1.4 mmol) in Toluene (20 mL) was heated to reflux overnight. The reaction mixture was poured into saturated sodium bicarbonate (100 mL), and ethyl acetate (150 mL) was subsequently added. The organic phase was separated, dried over anhydrous sodium sulfate, and concentrated. The crude material was purified by silica gel chromatography (0-45% EtOAC/hexanes) to afford the title compound (0.95 g, 36% yield). LCMS calc. for C₉H₁₁FNO₂ [M+H]⁺: m/z=184.1; Found: 184.0

### Step 3: (6aS,8R)-8-((5-(1,3-Dioxolan-2-yl)-3-methylpyridin-2-yl)oxy)-2-chloro-6a-ethyl-5,6,6a,7,8,9-hexahydropyrrolo[1′,2′:4,5]pyrazino[2,3-c]pyridazine

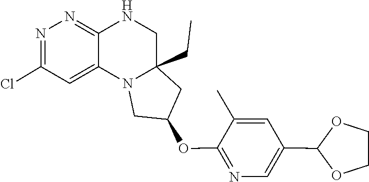 [SMILES: CC[C@]12CNc3nnc(Cl)cc3N1C[C@H](Oc1ncc(C3OCCO3)cc1C)C2]

The title compound was synthesized by procedures analogous to those outlined in Example 19, Step 4 using (6aS,8R)-2-chloro-6a-ethyl-5,6,6a,7,8,9-hexahydropyrrolo[1′,2′:4,5]pyrazino[2,3-c]pyridazin-8-ol instead of (6aS,8R)-6a-ethyl-2-(2-((2-methoxyethoxy)methoxy)phenyl)-5, 6,6a,7,8,9-hexahydropyrrolo[1′,2′:4,5]pyrazino[2,3-c]pyridazin-8-ol. LCMS calc. for C₂₀H₂₅ClN₅O₃[M+H]⁺: m/z=418.2; Found: 418.1.

### Step 4: (2-((6aS,8R)-8-((5-(1,3-Dioxolan-2-yl)-3-methylpyridin-2-yl)oxy)-6a-ethyl-5,6,6a,7,8,9-hexahydropyrrolo[1′,2′:4,5]pyrazino[2,3-c]pyridazin-2-yl)phenoxy)methyl tert-butyl hydrogen phosphate

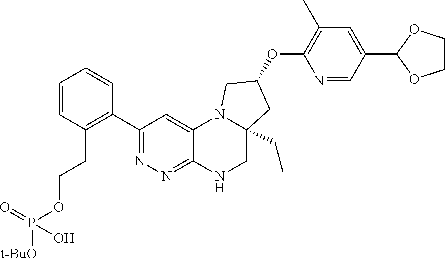 [SMILES: CC[C@]12CNc3nnc(-c4ccccc4CCOP(=O)(O)OC(C)(C)C)cc3N1C[C@H](Oc1ncc(C3OCCO3)cc1C)C2]

A mixture of (2-bromophenoxy)methyl di-tert-butyl phosphate (400 mg, 1.01 mmol), tetrahydroxydiboron (272 mg, 3.04 mmol), potassium acetate (298 mg, 3.04 mmol), XPhos Pd G4 (17.4 mg, 20.2 μmol) 2-(dicyclohexylphosphino)-2′,4′,6′-tri-isopropyl-1,1′-biphenyl (XPhos) (19.3 mg, 40.5 μmol) in EtOH (20.2 mL) was sparged with nitrogen then heated to 80° C. for 5 h. The reaction mixture was cooled to rt then (6aS,8R)-8-((5-(1,3-dioxolan-2-yl)-3-methylpyridin-2-yl)oxy)-2-chloro-6a-ethyl-5,6,6a,7,8,9-hexahydropyrrolo[1′,2′:4,5]pyrazino[2,3-c]pyridazine (240 mg, 0.574 mmol), potassium carbonate (238 mg, 1.72 mmol), XPhos Pd G4 (9.88 mg, 11.5 μmol) 2-(dicyclohexylphosphino)-2′,4′,6′-tri-isopropyl-1,1′-biphenyl (XPhos) (11.0 mg, 40.0 μmol), and water (6.5 mL) were added. The mixture was sparged with nitrogen and heated to 75° C. for 5 h. The reaction mixture was cooled to rt and diluted with DCM/MeOH (6:1) (v/v) (30 ml). The resulting two phases were separated and the organic layer was washed with water (2×20 mL). The organic layer was dried over sodium sulfate, filtered, and concentrated. The crude residue was purified by silica gel chromatography using (0-20% MeOH/DCM) to afford the title compound (80.0 mg, 0.125 mmol 21.7% yield) as a as a brown solid. LCMS calc. for C₃₁H₄₁N₅O₈P [M+H]⁺: m/z=642.3; Found: 642.1.

### Step 5: 4—((S)-2-((S)-2-((((9H-Fluoren-9-yl)methoxy)carbonyl)amino)-3-methylbutanamido)-5-ureidopentanamido)benzyl (6aS,8R)-2-(2-(((tert-butoxy(hydroxy)phosphoryl)oxy)methoxy)phenyl)-6a-ethyl-8-((5-formyl-3-methylpyridin-2-yl)oxy)-6a,7,8,9-tetrahydropyrrolo[1′,2′:4,5]pyrazino[2,3-c]pyridazine-5(6H)-carboxylate

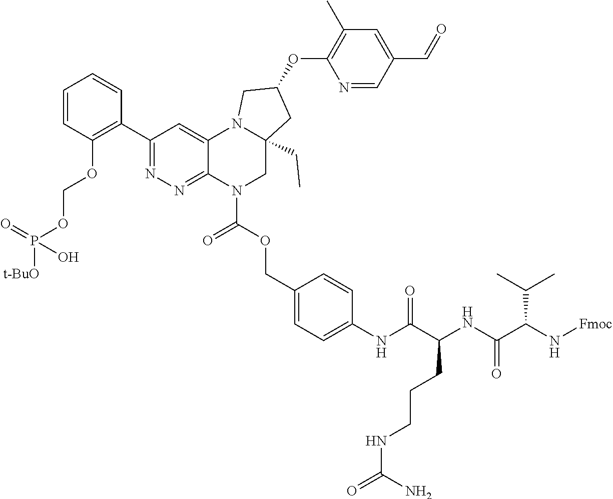

The title compound was synthesized by procedures analogous to those outlined in Example 21, Step 4 using N,N-diisopropylethyl amine instead of 2,6-lutidine. Note: Concomitant loss of the glycol protecting group was observed following prep-HPLC purification. LCMS calc. for C₆₃H₇₄N₁₀O₁₄P [M+H]⁺: m/z=1225.5; Found: 1225.5.

### Step 6: 4—((S)-2-((S)-2-Amino-3-methylbutanamido)-5-ureidopentanamido)benzyl (6aS,8R)-8-((5-((4-(2-((S)-2,6-dioxopiperidin-3-yl)-3-oxoisoindolin-5-yl)piperazin-1-yl)methyl)-3-methylpyridin-2-yl)oxy)-6a-ethyl-2-(2-((phosphonooxy)methoxy)phenyl)-6a,7,8,9-tetrahydropyrrolo[1′,2′:4,5]pyrazino[2,3-c]pyridazine-5(6H)-carboxylate

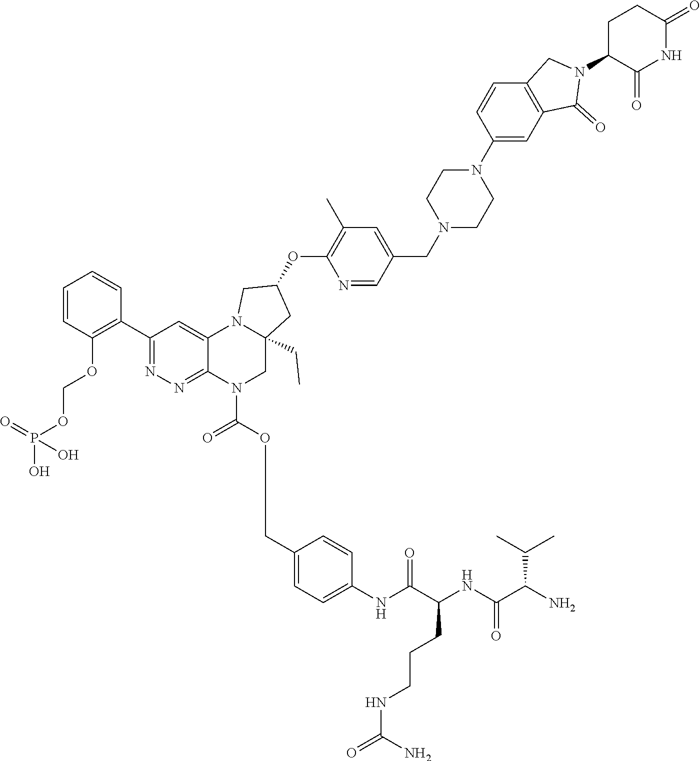 [SMILES: CC[C@@]12C[C@@H](Oc3ncc(CN4CCN(c5ccc6c(c5)C(=O)N([C@H]5CCC(=O)NC5=O)C6)CC4)cc3C)CN1c1cc(-c3ccccc3OCOP(=O)(O)O)nnc1N(C(=O)OCc1ccc(NC(=O)[C@H](CCCNC(N)=O)NC(=O)[C@@H](N)C(C)C)cc1)C2]

A solution of (S)-3-(1-oxo-6-(piperazin-1-yl)isoindolin-2-yl)piperidine-2,6-dione (20.5 mg, 46.3 μmol), 4-((S)-2-((S)-2-((((9H-fluoren-9-yl)methoxy)carbonyl)amino)-3-methylbutanamido)-5-ureidopentanamido)benzyl (6aS,8R)-8-((5-(1,3-dioxolan-2-yl)-3-methylpyridin-2-yl)oxy)-2-(2-(((tert-butoxy(hydroxy)phosphoryl)oxy)methoxy)phenyl)-6a-ethyl-6a,7,8,9-tetrahydropyrrolo[1′,2′:4,5]pyrazino[2,3-c]pyridazine-5(6H)-carboxylate (23.0 mg, 18.1 μmol), and N,N-diisopropylethylamine (32.7 μL, 0.180 mmol) in dimethyl sulfoxide (1.9 mL) was stirred at rt for 5 min. Acetic acid (10.7 μL, 0.180 mmol) was added to the reaction mixture and stirred for 5 min before addition of sodium triacetoxyborohydride (15.9 mg, 0.08 mmol). The reaction mixture was stirred at rt for 1 h before addition of N,N-diisopropylethylamine (32.7 μL, 0.18 mmol), acetic acid (10.7 μL, 0.18 mmol), and sodium triacetoxyborohydride (15.9 mg, 75.0 μmol) were added. The reaction mixture was stirred at rt overnight before addition of a further amount of acetic acid (10.7 μL, 0.180 mmol) and N,N-diisopropylethylamine (32.7 μL, 0.180 mmol). The reaction mixture was stirred at rt for 12 h. The crude reaction was diluted with acetonitrile/water (10:1) (v/v) and purified directly by prep-HPLC on a C18 column (21-40% MeCN/0.2% TFA (aq.)) the TFA salt of the title compound (4.00 mg, 2.69 μmol, 17.0% yield) as a white solid. LCMS calc. for C₆₁H₇₇N₁₄O₁₄P [M+2H]2+: m/z=630.3; Found: 630.4.

### Step 7: 4—((S)-2-((S)-2-(6-(2,5-dioxo-2,5-dihydro-1H-pyrrol-1-yl)hexanamido)-3-methylbutanamido)-5-ureidopentanamido)benzyl (6aS,8R)-8-((5-((4-(2-((S)-2,6-Dioxopiperidin-3-yl)-3-oxoisoindolin-5-yl)piperazin-1-yl)methyl)-3-methylpyridin-2-yl)oxy)-6a-ethyl-2-(2-((phosphonooxy)methoxy)phenyl)-6a,7,8,9-tetrahydropyrrolo[1′,2′:4,5]pyrazino[2,3-c]pyridazine-5(6H)-carboxylate

The title compound was synthesized by procedures analogous to those outlined in Example 19, Step 8 using 6-(2,5-dioxo-2,5-dihydro-1H-pyrrol-1-yl)hexanoic acid instead of (6-(2,5-dioxo-2,5-dihydro-1H-pyrrol-1-yl)hexanoyl)glycylglycyl-L-phenylalanine. LCMS calc. for C₇₁H₈₈N₁₅O₁₇P [M+2H]2+: m/z=726.8; Found: 727.1.

### Example 26. Preparation of Additional Degrader-Linker Compounds

Additional Degrader-Linker compounds described herein were synthesized and were analyzed using liquid chromatography-mass spectrometry (LCMS). LCMS results are summarized below in Table 3.

TABLE 3
LCMS Data for Degrader-Linker Compounds.

| Compound No. | Observed Ion | m/z |
|---|---|---|
| 2.01 | [M + H]⁺ | 1709.0 |
| 2.02 | [M + H]⁺ | 1576.7 |
| 2.03 | [M + H]⁺ | 1590.8 |
| 2.04 | [M + H]⁺ | 1340.7 |
| 2.05 | [M + H]⁺ | 1522.8 |
| 2.06 | [M + H]⁺ | 1591.9 |
| 2.07 | [M + H]⁺ | 1561.6 |
| 2.08 | [M + 2H]²⁺ | 673.1 |
| 2.09 | [M + 2H]²⁺ | 657.5 |
| 2.10 | [M + 2H]²⁺ | 672.2 |
| 2.11 | [M + 2H]²⁺ | 1114.4 |
| 2.12 | [M + H]⁺ | 1521.7 |
| 2.13 | [M + 2H]²⁺ | 658.8 |
| 2.14 | [M + 2H]²⁺ | 781.8 |
| 2.15 | [M + H]⁺ | 1517.6 |
| 2.16 | [M + H]⁺ | 1518.6 |
| 2.17 | [M + H]⁺ | 1661.5 |
| 2.18 | [M + 2H]²⁺ | 702.6 |
| 2.19 | [M + H]⁺ | 1157.4 |
| 2.20 | [M + H]⁺ | 1526.7 |
| 2.21 | [M + H]⁺ | 1501.7 |
| 2.22 | [M + H]⁺ | 1264.5 |
| 2.23 | [M + H]⁺ | 1592.0 |
| 2.24 | [M + 2H]²⁺ | 728.5 |
| 2.25 | [M + H]⁺ | 1127.5 |
| 2.26 | [M + H]⁺ | 1127.4 |
| 2.27 | [M + 2H]²⁺ | 762.3 |
| 2.28 | [M + H]⁺ | 1155.4 |
| 2.29 | [M + H]⁺ | 1156.3 |
| 2.30 | [M + 2H]²⁺ | 899.8 |
| 2.31 | [M + H]⁺ | 1156.5 |
| 2.32 | [M + H]⁺ | 1115.3 |
| 2.33 | [M + H]⁺ | 1115.2 |
| 2.34 | [M + H]⁺ | 1115.4 |
| 2.35 | [M + H]⁺ | 1417.7 |
| 2.36 | [M + H]⁺ | 1115.3 |
| 2.37 | [M + 2H]²⁺ | 783.5 |
| 2.38 | [M + H]⁺ | 1503.6 |
| 2.39 | [M + H]⁺ | 1549.7 |
| 2.40 | [M + H]⁺ | 1505.7 |
| 2.41 | [M + H]⁺ | 1583.8 |
| 2.42 | [M + H]⁺ | 1575.6 |
| 2.43 | [M + 2H]²⁺ | 691.3 |
| 2.44 | [M + H]⁺ | 1537.7 |
| 2.45 | [M + H]⁺ | 1589.7 |
| 2.46 | [M + H]⁺ | 1704.7 |
| 2.47 | [M + H]⁺ | 1807.9 |
| 2.48 | [M + 2H]²⁺ | 688.4 |
| 2.49 | [M + H]⁺ | 1562.1 |
| 2.50 | [M + H]⁺ | 1447.6 |
| 2.51 | [M + 2H]²⁺ | 997.0 |
| 2.52 | [M + H]⁺ | 1608.7 |
| 2.53 | [M + H]⁺ | 1662.9 |
| 2.54 | [M + 2H]²⁺ | 715.4 |
| 2.55 | [M + 2H]²⁺ | 1537.9 |
| 2.56 | [M + H]⁺ | 1530.8 |
| 2.57 | [M + 2H]²⁺ | 1893.1 |
| 2.58 | [M + 2H]²⁺ | 1247.5 |
| 2.59 | [M + 2H]²⁺ | 723.5 |
| 2.60 | [M + H]⁺ | 1457.7 |
| 2.61 | [M + 2H]²⁺ | 643.1 |
| 2.62 | [M + 2H]²⁺ | 752.2 |
| 2.63 | [M + 2H]²⁺ | 793.1 |
| 2.65 | [M + 2H]²⁺ | 796.3 |
| 2.66 | [M + 3H]³⁺ | 1257.1 |
| 2.67 | [M + H]¹⁺ | 1606.4 |
| 2.68 | [M + H]¹⁺ | 1743.5 (100.0%), |
| | | 1745.0 (90.0%) |
| | | (most prevalent |
| | | observed isotopes) |
| 2.69 | [M + H]¹⁺ | 1864.4 |
| 2.70 | [M + H]¹⁺ | 1671.6 |
| 2.71 | [M + H]¹⁺ | 1583.3 |
| 2.72 | [M + H]¹⁺ | 1672.7 |
| 2.73 | [M + H]¹⁺ | 1637.7 |
| 2.74 | [M + H]¹⁺ | 1708.9 |
| 2.76 | [M + H]¹⁺ | 1459.4 |
| 2.77 | [M + H]¹⁺ | 1704.6 |
| 2.78 | [M + 2H]²⁺ | 884.5 |
| 2.79 | [M + 2H]²⁺ | 688.4 |
| 2.85 | [M + 2H]²⁺ | 727.1 |

### Example 27. 1H-NMR Spectra of Degrader-Linker Compounds

Nuclear magnetic resonance (NMR) spectra were recorded by using a 400-MHz (Bruker) NMR spectrometer. Chemical shifts are reported in ppm (δ) referenced to tetramethylsilane (6=0.00 ppm), or dimethyl sulfoxide (6=2.50 ppm). The chemical shifts of representative peaks are tabulated.

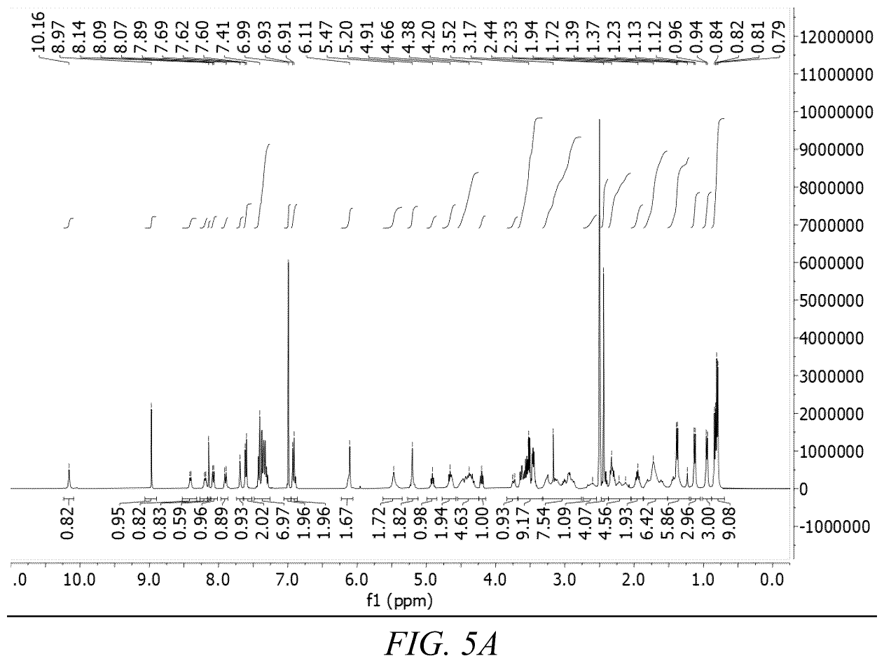

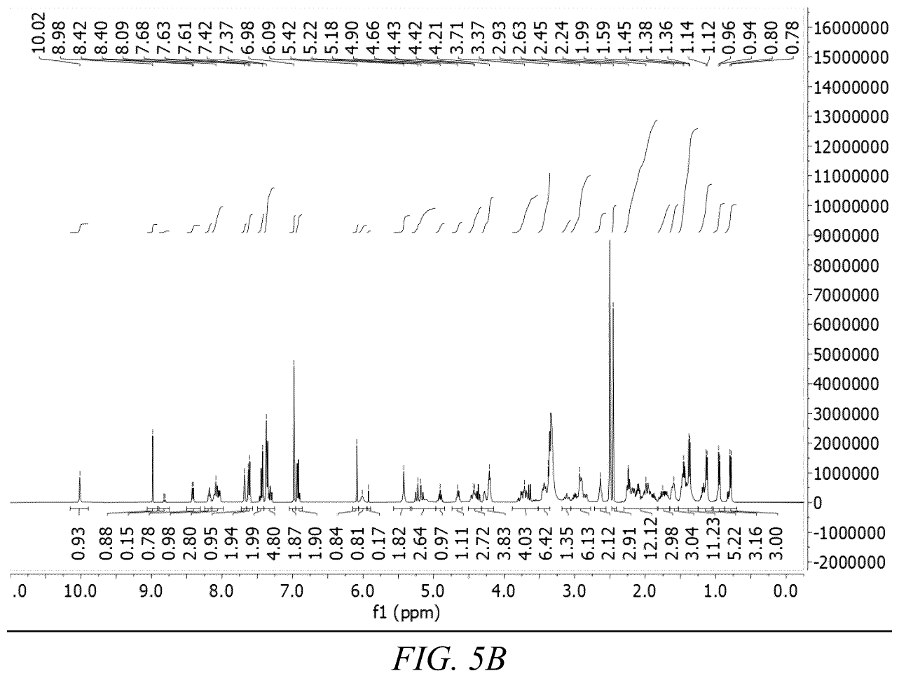

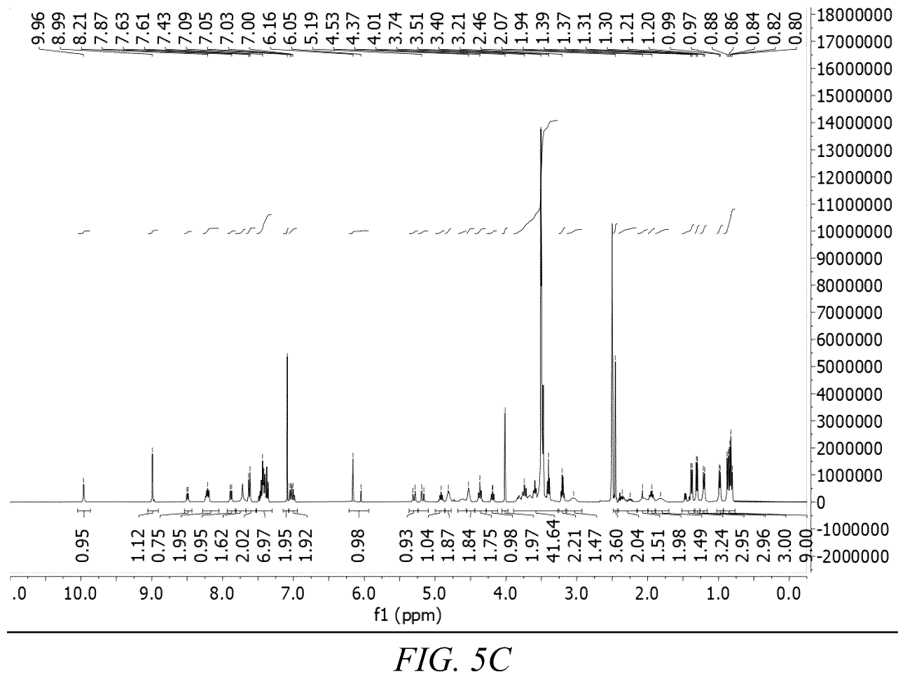

A ¹H-NMR spectrum of Compound 2.63 is shown in FIG. 5A. ¹H-NMR (400 MHz, DMSO-d₆) exemplary peaks in ppm (δ): 10.16 (s, 1H), 8.97 (s, 1H), 8.41 (d, J=7.5 Hz, 1H), 8.19 (d, J=7.3 Hz, 1H), 8.14 (s, 1H), 8.08 (d, J=8.1 Hz, 1H), 7.90 (d, J=8.8 Hz, 1H), 7.69 (s, 1H), 7.61 (d, J=8.3 Hz, 2H), 7.48-7.19 (m, 7H), 6.99 (s, 2H), 6.95-6.85 (m, 2H), 6.19-6.01 (m, 2H), 5.95 (s, 0.05, minor rotamer), 5.75-5.36 (m, 2H), 5.20 (s, 2H), 4.91 (p, J=6.9 Hz, 1H), 4.76-4.58 (m, 2H), 4.45-4.26 (m, 5H), 4.20 (dd, J=8.6, 6.7 Hz, 1H), 3.74 (d, J=11.3 Hz, 1H), 2.71-2.52 (m, 1H), 2.48-2.38 (m, 4H), 2.37-2.04 (m, 5H), 2.03-1.88 (m, 1H), 1.87-1.52 (m, 6H), 1.50-1.20 (m, 6H), 1.13 (d, J=6.9 Hz, 3H), 0.95 (d, J=6.4 Hz, 3H), 0.83 (d, J=6.7 Hz, 3H), 0.80 (d, J=6.7 Hz, 6H).

A ¹H-NMR spectrum of Compound 2.65 is shown in FIG. 5B. ₁H-NMR (400 MHz, DMSO-d₆) exemplary peaks in ppm (δ): 10.02 (s, 1H), 8.98 (s, 1H), 8.82 (d, J=7.7 Hz, 0.2H, minor rotamer), 8.41 (d, J=7.7 Hz, 1H), 8.18 (t, J=5.8 Hz, 1H), 8.14-7.97 (m, 3H), 7.68 (s, 1H), 7.62 (d, J=8.3 Hz, 2H), 7.43 (d, J=8.3 Hz, 2H), 7.39-7.25 (m, 5H), 6.98 (s, 2H), 6.93 (d, J=7.9 Hz, 2H), 6.09 (s, 0.85H, major rotamer), 6.01 (bs, 1H), 5.92 (s, 0.1H, minor rotamer), 5.42 (s, 2H), 5.28-4.98 (m, 3H), 4.95-4.84 (m, 1H), 4.74-4.57 (m, 1H), 4.50-4.33 (m, 3H), 4.31-4.15 (m, 4H), 2.69-2.57 (m, 2H), 2.45 (s, 3H), 2.30-1.82 (m, 12H), 1.82-1.65 (m, 3H), 1.65-1.53 (m, 3H), 1.53-1.27 (m, 11H), 1.24-1.08 (m, 5H), 1.03-0.90 (m, 3H), 0.85-0.75 (m, 3H).

A ¹H-NMR spectrum of Compound 2.69 is shown in FIG. 5C. ¹H-NMR (400 MHz, DMSO-d₆) exemplary peaks in ppm (δ): 9.96 (s, 1H), 9.02-8.95 (m, 1H), 8.50 (d, J=7.6 Hz, 1H), 8.26-8.10 (m, 2H), 7.88 (d, J=8.5 Hz, 1H), 7.72 (s, 2H), 7.62 (d, J=8.4 Hz, 2H), 7.55-7.37 (m, 7H), 7.09 (s, 2H), 7.08-6.92 (m, 2H), 6.16 (s, 0.85H, major rotamer), 6.05 (s, 0.15H, minor rotamer), 5.30 (d, J=12.7 Hz, 1H), 5.17 (d, J=12.7 Hz, 1H), 4.91 (p, J=6.9 Hz, 1H), 4.86-4.68 (m, 2H), 4.67-4.45 (m, 3H), 4.37 (t, J=7.4 Hz, 2H), 4.19 (dd, J=8.5, 6.8 Hz, 1H), 4.01 (s, 2H), 3.20 (q, J=5.8 Hz, 2H), 3.11-2.95 (m, 2H), 2.48-2.42 (m, 4H), 2.41-2.15 (m, 2H), 2.13-1.99 (m, 2H), 1.99-1.89 (m, 2H), 1.89-1.69 (s, 2H), 1.47 (d, J=6.9 Hz, 0.6H, minor rotamer), 1.38 (d, J=7.0 Hz, 2.4H, major rotamer), 1.30 (d, J=7.0 Hz, 3H), 1.20 (d, J=6.9 Hz, 3H), 0.98 (d, J=6.5 Hz, 3H), 0.90-0.76 (m, 9H).

### Example 28. SMARCA2 HiBiT and SMARCA4 HiBiT Degradation Assay

### Preparation of SMARCA2 4-HiBiT Knock-In Cells

HiBiT peptide knock-in of SMARCA2 in LgBiT expressing HEK293T cells was performed by CRISPR-mediated tagging system as described Promega. The homozygous HiBiT knock-in on c-terminus SMARCA2 was confirmed by sanger sequence. SMARCA2-HiBiT knock-in Hela monoclonal cell (CS302366) and SMARCA4-HiBiT knock-in Hela monoclonal cell (CS3023226) were purchased from Promega. The heterozygous HiBiT-knock-in was confirmed by sanger sequence in both SMARCA2-HiBiT and SMARCA4-HiBiT monoclonal cells.

### Smarca2 HiBiT and Smarca4 HiBiT Degradation Assay in HeLa Cells

Dispense 10ul aliquot of prepared Hela-SMARCA2-HiBiT or Hela-SMARCA4-HiBiT cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 2500 cells/well @20 μL/well. Transfer Hela-HiBiT cells to 50 mL conical (#430290, Corning). Spin down at 1000 rpm for 5 min using tabletop centrifuge (SPINCHRON 15, Beckman). Discard supernatant and resuspend cell pellet in modified EMEM (#30-2003, ATCC) cell culture media containing 10% FBS (F2422-500ML, Sigma), and 1× Penicillin/Streptomycin (200 g/L) (30-002-CI, Corning) to a cell density of 125,000 cells/mL. Dispense 20 mL of resuspended Hela-HiBit cells per well in 384-well TC treated plate (#12-565-343, Thermo Scientific) using standard cassette (#50950372, Thermo Scientific) on Multidrop Combi (#5840310, Thermo Scientific) inside laminar flow cabinet.

The degrader compounds of Table 4 were dissolved in DMSO to make 10 mM stock and 3-fold series dilutions were further conducted keeping the highest concentration 10 μM. Dispense test compounds onto plates using digital liquid dispenser (D300E, Tecan). Incubate plates in humidified tissue culture incubator at 37° C. for 18 hours. Add 20 mL of prepared Nano-Glo® HiBiT Lytic detection buffer (N3050, Promega) to each well of 384-well plate using small tube cassette (#24073295, Thermo Scientific) on Multidrop Combi, incubate at room temperature for 30-60 min. Read plates on microplate reader (Envision 2105, PerkinElmer) using 384 well Ultra-Sensitive luminescence mode. Raw data files and compound information reports are swept into centralized data lake and deconvoluted using automated scripts designed by TetraScience, Inc. Data analysis, curve-fitting and reporting done in Dotmatics Informatics Suite using Screening Ultra module. Results are summarized below in Table 5.

TABLE 4
Degrader Compounds

| Degrader | |
|---|---|
| Compound | |
| No. | Degrader Compound Structure |
| 1 | 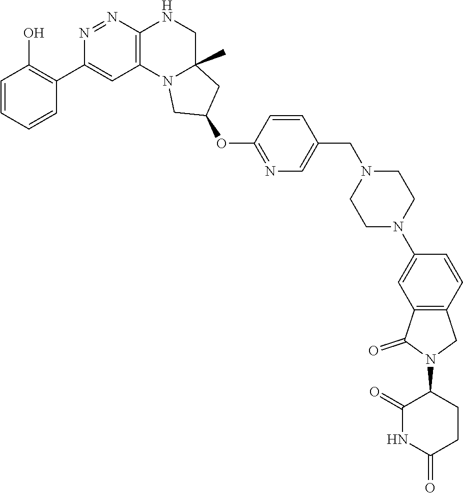 [SMILES: C[C@]12CNc3nnc(-c4ccccc4O)cc3N1C[C@H](Oc1ccc(CN3CCN(c4ccc5c(c4)C(=O)N([C@H]4CCC(=O)NC4=O)C5)CC3)cn1)C2] |
| 2 | 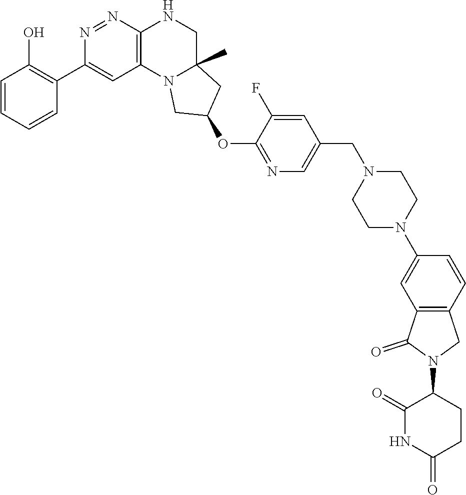 [SMILES: C[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)cc1F)C2] |
| 3 | 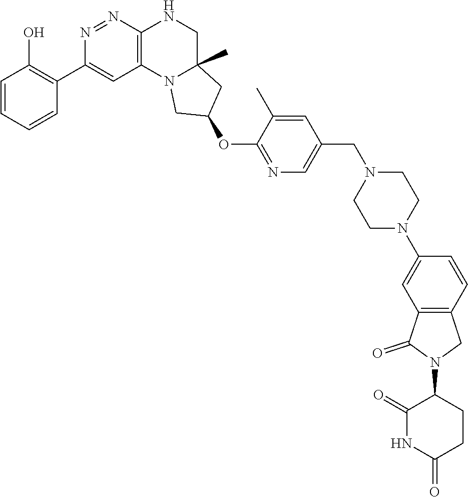 [SMILES: Cc1cc(CN2CCN(c3ccc4c(c3)C(=O)N([C@H]3CCC(=O)NC3=O)C4)CC2)cnc1O[C@H]1CN2c3cc(-c4ccccc4O)nnc3NC[C@]2(C)C1] |
| 4 | 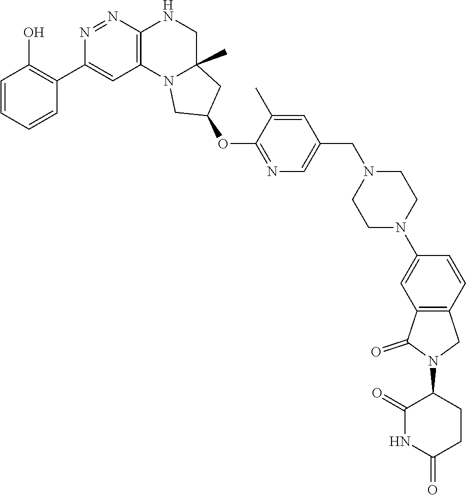 [SMILES: Cc1cc(CN2CCN(c3ccc4c(c3)C(=O)N([C@H]3CCC(=O)NC3=O)C4)CC2)cnc1O[C@H]1CN2c3cc(-c4ccccc4O)nnc3NC[C@]2(C)C1] |
| 5 | 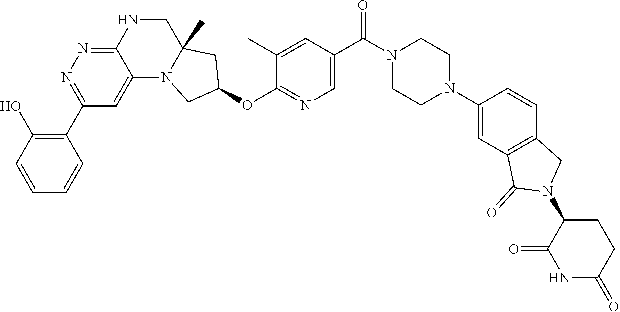 [SMILES: Cc1cc(C(=O)N2CCN(c3ccc4c(c3)C(=O)N([C@H]3CCC(=O)NC3=O)C4)CC2)cnc1O[C@H]1CN2c3cc(-c4ccccc4O)nnc3NC[C@]2(C)C1] |
| 6 | 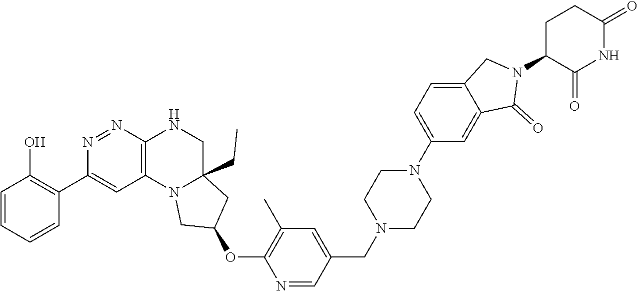 [SMILES: CC[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)cc1C)C2] |
| 7 | 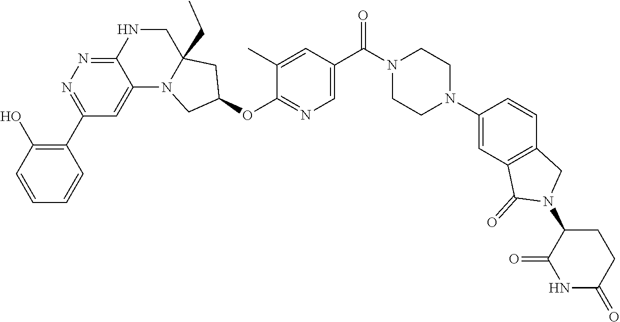 [SMILES: CC[C@]12CNc3nnc(-c4ccccc4O)cc3N1C[C@H](Oc1ncc(C(=O)N3CCN(c4ccc5c(c4)C(=O)N([C@H]4CCC(=O)NC4=O)C5)CC3)cc1C)C2] |
| 8 | 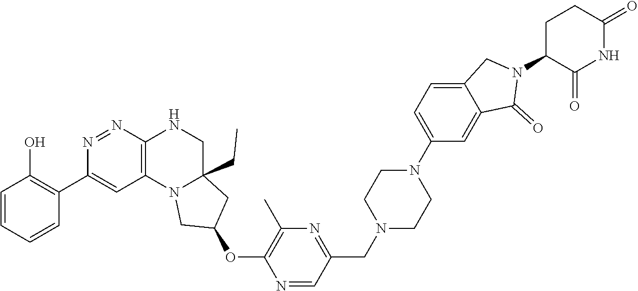 [SMILES: CC[C@]12CNc3nnc(-c4ccccc4O)cc3N1C[C@H](Oc1ncc(CN3CCN(c4ccc5c(c4)C(=O)N([C@H]4CCC(=O)NC4=O)C5)CC3)nc1C)C2] |
| 9 | 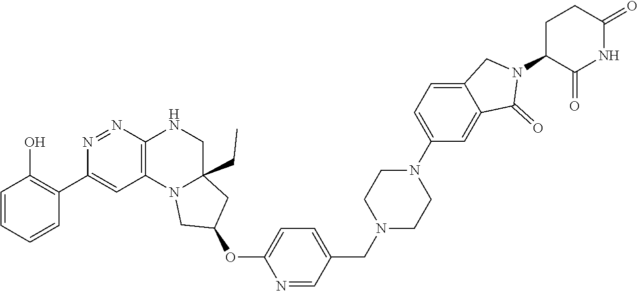 [SMILES: CC[C@]12CNc3nnc(-c4ccccc4O)cc3N1C[C@H](Oc1ccc(CN3CCN(c4ccc5c(c4)C(=O)N([C@H]4CCC(=O)NC4=O)C5)CC3)cn1)C2] |
| 10 | 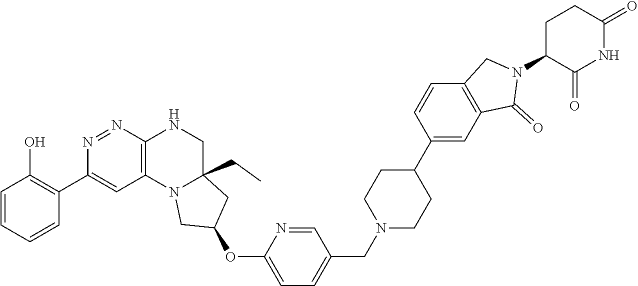 [SMILES: CC[C@]12CNc3nnc(-c4ccccc4O)cc3N1C[C@H](Oc1ccc(CN3CCC(c4ccc5c(c4)C(=O)N([C@H]4CCC(=O)NC4=O)C5)CC3)cn1)C2] |
| 11 | 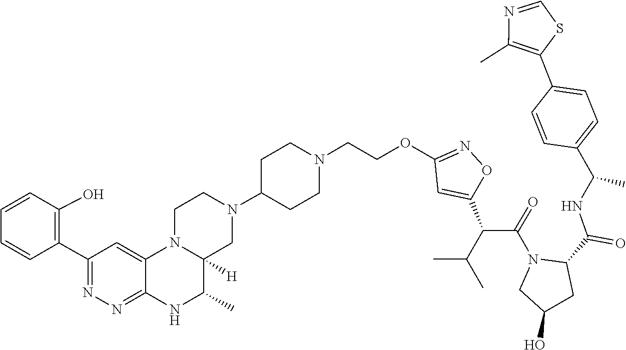 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@@H](C)[C@@H]5C4)CC3)no2)C(C)C)cc1] |
| 12 | 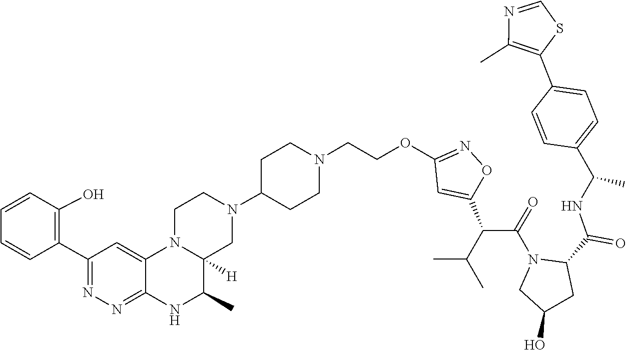 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@H](C)[C@@H]5C4)CC3)no2)C(C)C)cc1] |
| 13 | 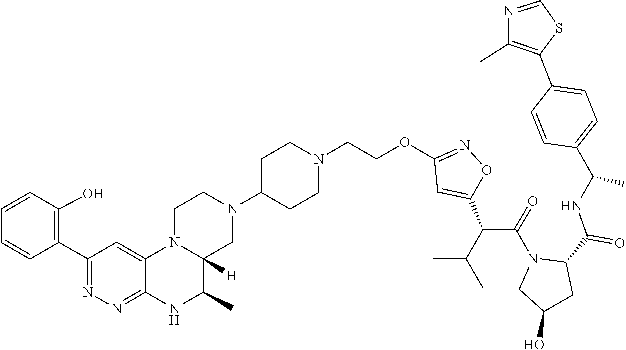 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@H](C)[C@H]5C4)CC3)no2)C(C)C)cc1] |
| 14 | 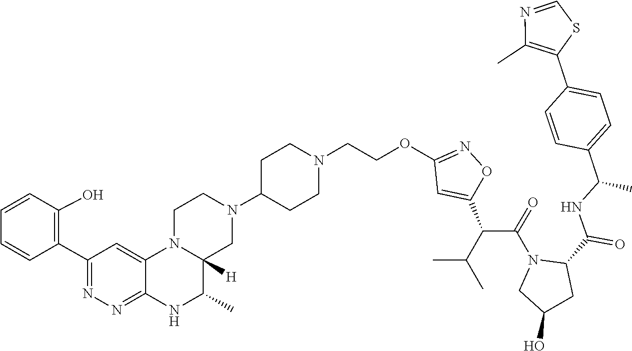 [SMILES: Cc1ncsc1-c1ccc([C@H](C)NC(=O)[C@@H]2C[C@@H](O)CN2C(=O)[C@@H](c2cc(OCCN3CCC(N4CCN5c6cc(-c7ccccc7O)nnc6N[C@@H](C)[C@H]5C4)CC3)no2)C(C)C)cc1] |

TABLE 5
SMARCA2 and SMARCA4 Degradation

| Degrader | SMARCA2 | SMARCA2 | SMARCA4 | SMARCA4 |
|---|---|---|---|---|
| Compound | DC₅₀ | Dₘₐₓ | DC₅₀ | Dₘₐₓ |
| 1 | A | A | B | A |
| 2 | B | A | B | A |
| 3 | B | A | B | B |
| 4 | A | A | B | A |
| 5 | A | A | B | B |
| 6 | A | A | A | A |
| 7 | A | A | B | B |
| 8 | A | A | B | A |
| 9 | A | A | B | A |
| 10 | B | A | B | B |
| 11 | B | A | C | A |
| 12 | C | A | C | A |
| 13 | B | A | C | A |
| 14 | C | B | C | B |

In Table 5, columns DC₅₀, an “A” denotes DC₅₀<0.1 nM; a “B” denotes 0.1 nM<DC₅₀<1 nM; a “C” denotes 1.0 nM<DC₅₀<10 nM; a “D” denotes DC₅₀≥10 nM. In Table 5, columns Dₘₐₓ, an “A” denotes Dₘₐₓ>90%; a “B” denotes 50%<Dₘₐₓ≤90%; a “C” denotes that a Dₘₐₓ≤50%.

### Example 29. ADC Preparation and In Vitro Evaluation

### Bioconjugation Procedure

A solution of payload-linker (containing a thiol-reactive group) in DMSO (1.25 or 10 mM) was added to a solution of reduced mono-clonal antibody in PBS pH 7.4. The reaction was incubated or gently swirled until completion (40 min to 24 h) to afford the desired ADCs.

For ADC Nos. 1.85A and 1.85B listed in Table 6, the solution of reduced mono-clonal antibody was buffer exchanged using Amicon Ultracel 15 centrifugal spin filters (Millipore) with 50 k MWCO at 3260 g for 4 cycles prior to incubation with the solution of payload-linker (containing a thiol-reactive group).

ADC Nos. 1.40, 1.38A, 1.15, 1.09, 1.13, and 1.38B listed in Table 6 were optionally purified by prep-HPLC on a SEC column (Waters PremierSEC 250 Å) or sterilized with a 0.25 mm sterile filter. The ADCs were formulated into PBS pH 7.4, concentrated by centrifugal ultrafiltration, and held at 4° C. for storage.

Each of the remaining ADCs listed in Table 6 were optionally prepared using one of the following methods: (i) ADCs were purified by Akta on a HIC column (butyl HP), buffer exchanged into 1×PBS buffer pH 7.4, concentrated with Amicon 50 kD MWCO, spiked with sucrose (6% w:v), adjusted to the desired concentration (e.g., 1 mg/mL), and held at −20° C. for storage. (ii) ADCs were buffer exchanged by using Amicon Ultracel 15 centrifugal spin filters (Millipore) with 50 k MWCO at 3260 g for 4 cycles.

The ADCs listed in Table 6 were prepared according to the methods described above using the indicated antibody and payload linker compound. Antibody A is a humanized anti-PSMA antibody (Creative Biolabs). Gemtuzumab is a commercially available anti-CD33 antibody. Rosopatamab is a commercially available anti-PSMA antibody, sacituzumab is a commercially available anti-TROP2 antibody, tusamitamab is a commercially available anti-CEACAM5 antibody, telisotuzumab is a commercially available anti-c-MET antibody, human IgG1 Kapa is a commercially available isotype control (MedChemExpress), pivekimab is a commercially available anti-CD123 antibody, mirzotamab is a commercially available anti-B7-H3 antibody, Antibody B is a commercially available anti-CALR antibody (Novus Biologicals: Catalog #: NBP2-50053), and human IgG2b Kapa is a commercially available isotype control (MedChemExpress).

TABLE 6
List of ADCs

| | | Degrader-Linker | |
|---|---|---|---|
| ADC No. | mAb | Compound No. | DAR |
| 1.40 | Antibody A | 2.40 | 4.6 |
| 1.38A | Antibody A | 2.38 | 4.0 |
| 1.15 | Antibody A | 2.15 | 3.1 |
| 1.09 | Antibody A | 2.09 | 3.1 |
| 1.13 | Antibody A | 2.13 | 3.6 |
| 1.38B | gemtuzumab | 2.38 | 4.7 |
| 1.59 | Antibody A | 2.59 | 4.3 |
| 1.60 | Antibody A | 2.60 | 4.1 |
| 1.10A | rosopatamab | 2.10 | 2.0 |
| 1.08 | rosopatamab | 2.08 | 2.0 |
| 1.38C | sacituzumab | 2.38 | 3.1 |
| 1.10B | sacituzumab | 2.10 | 2.0 |
| 1.38D | tusamitamab | 2.38 | 3.3 |
| 1.10C | tusamitamab | 2.10 | 2.0 |
| 1.38E | rosopatamab | 2.38 | 3.6 |
| 1.61A | tusamitamab | 2.61 | 2.1 |
| 1.61B | sacituzumab | 2.61 | 2.0 |
| 1.61C | human IgG1 Kappa | 2.61 | 2.0 |
| | isotype control | | |
| 1.61D | telisotuzumab | 2.61 | 2.0 |
| 1.62 | rosopatamab | 2.62 | 4.1 |
| 1.10D | pivekimab | 2.10 | 2.0 |
| 1.61E | mirzotamab | 2.61 | 2.6 |
| 1.41 | rosopatamab | 2.41 | 4.3 |
| 1.45 | rosopatamab | 2.45 | 3.8 |
| 1.18 | rosopatamab | 2.18 | 4.1 |
| 1.07 | rosopatamab | 2.07 | 2.8 |
| 1.02 | rosopatamab | 2.02 | 4.0 |
| 1.35 | rosopatamab | 2.35 | 4.1 |
| 1.65 | rosopatamab | 2.65 | 3.4 |
| 1.66 | rosopatamab | 2.66 | 4.0 |
| 1.67 | rosopatamab | 2.67 | 4.0 |
| 1.68 | rosopatamab | 2.68 | 3.2 |
| 1.79 | rosopatamab | 2.79 | 4.0 |
| 1.85A | Antibody B | 2.85 | 2.7 |
| 1.85B | human IgG2b Kappa | 2.85 | 2.9 |
| | isotype control | | |

### Internalization

Efficient internalization was observed for ADC No. 1.38A in PSMA-positive prostate cancer cell lines, but not in PSMA-negative prostate cancer cell lines.

### In-Cell Western Blot for SMARCA2 and SMARCA4 Degradation in LNCAP and PC3 Cell Lines

The LNCAP (PSMA positive) and PC3 (PSMA negative) cell lines were obtained from ATCC. All cells were cultured in either RPMI1640, EMEM, or F-12K media, supplemented with 10% FBS and 1% penicillin/streptomycin, and were incubated at 37° C. with 5% CO₂in a standard incubator. The harvested cells were re-suspended at a concentration of 1-3×10≡cells/ml, and 100 μL of the cell suspension was aliquoted to each well of 384-well plates with black walls and clear bottoms (Corning). Once the cells settled at the bottom (1-16 hours of incubation at 37° C.), antibody-drug conjugates were dispensed using the D300e Tecan digital dispense or IDOT (Dispendix). The plates were then placed in a humidified 37° C., 5% CO₂ incubator for 16-18 hours. After incubation, the cells were fixed with 4% paraformaldehyde or glyoxal for 30 minutes at room temperature. Cell permeabilization was achieved by washing four times (with a five-minute incubation at room temperature before each wash) with PBS containing 0.1% Triton X-100, followed by a 1-hour blocking using Odyssey blocking buffer (LiCOR). Following permeabilization and blocking, the cells were probed with the primary antibodies SMARCA2 (Cell Signaling Technology, #D9E8B) or SMARCA4 (Cell Signaling Technology, #D1Q7F) in Odyssey blocking buffer overnight at 4° C. on a rocker. Excess primary antibody was removed by five washes (with a five-minute incubation at room temperature before each wash) with PBS buffer containing 0.1% Triton X-100. All washed cells were then stained with the secondary antibody (800CW goat anti-rabbit IgG, LiCOR) and the nuclear staining reagent DRAQ5 (Abcam) for one hour at room temperature. Excess secondary antibody was removed by five washes (with a five-minute incubation at room temperature before each wash) with PBS buffer containing 0.1% Triton X-100 followed by a final wash with water. All wells were air-dried for 30 minutes at room temperature before scanning. Plate scanning was conducted using the LiCOR Odyssey CLx imaging system to capture integrated intensities at 700 nm and 800 nm channels. The percentage SMARCA2 or SMARCA4 degradation was calculated using the DMSO-only well as a control. The ratio of signals at the 800/700 nm was normalized to background (no treatment), and DC₅₀ and maximum inhibition (Dₘₐₓ) values were determined using a four-parameter logistic curve fit. DC₅₀ and Max inhibition (Dₘₐₓ) values were determined using GraphPad Prism v8.

Odyssey CLx imaging system to acquire integrated intensities at 700 nm and 800 nm channel and analyzed Image Studio software v5.2. Results are shown in Tables 7 and 8.

TABLE 7
LNCAP Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.40 | D | C | C | C |
| 1.38A | D | C | C | C |
| 1.15 | D | C | C | C |
| 1.09 | C | C | B | C |
| 1.13 | C | C | C | C |
| 1.59 | D | C | C | C |
| 1.60 | D | C | C | C |
| 1.10A | — | — | C | C |
| 1.08 | — | — | C | C |
| 1.38E | D | C | D | C |
| 1.61D | C | C | B | C |
| 1.61C | B | C | A | C |
| 1.62 | D | C | C | C |
| 1.61E | D | C | D | C |
| 1.41 | C | C | C | C |
| 1.45 | D | C | C | C |
| 1.18 | D | C | D | C |
| 1.07 | D | C | C | B |
| 1.02 | — | — | C | C |
| 1.35 | — | — | C | C |
| 1.65 | — | — | C | C |
| 1.66 | D | C | C | C |
| 1.67 | D | C | D | C |
| 1.68 | — | — | C | C |
| 1.79 | C | C | C | C |

TABLE 8
PC3 Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.40 | B | C | A | C |
| 1.38A | B | C | A | B |
| 1.15 | B | C | A | A |
| 1.09 | B | C | A | B |
| 1.13 | B | C | B | B |
| 1.59 | A | A | A | A |
| 1.60 | A | A | A | A |
| 1.38E | C | C | B | B |
| 1.62 | A | A | A | A |
| 1.41 | B | C | A | B |
| 1.45 | B | C | B | B |
| 1.18 | C | C | B | B |
| 1.07 | B | C | B | B |
| 1.02 | — | — | B | B |
| 1.65 | — | — | A | A |
| 1.66 | — | — | B | B |
| 1.67 | B | B | A | A |
| 1.68 | — | — | A | A |
| 1.79 | C | C | B | C |

In Tables 7 and 8, an “A” denotes a DC₅₀ value of >5000 ng/mL; a “B” denotes a DC₅₀ value of 500 ng/mL<DC₅₀<5000 ng/mL; a “C” denotes a DC₅₀ value of 50 ng/mL<DC₅₀<500 ng/mL; a “D” denotes a DC₅₀ value of <50 ng/mL.

In Tables 7 and 8, an “A” denotes a Dₘₐₓ<50%; a “B” denotes a Dₘₐₓ value of 50%<Dₘₐₓ≤75%; a “C” denotes a Dₘₐₓ >75%.

### Western Blot for SMARCA2/4 Degradation in MV411 Cell Line

MV411 (CD33 positive) cells were cultured under standard culture conditions (37° C., 5% CO₂) prior to the experiment. Cells were seeded in complete media (RPMI-1640, 10% Fetal Bovine Serum, 1× penicillin-streptomycin) in 12-well, clear-bottom plates (Corning, 353043) at a density of 1e⁶ cells per well. Cells were treated for 18 h with 1000 ng/mL of ADC formulated in PBS+0.3% tween+glycerol. Following treatment, cell suspensions were centrifuged, and pellets isolated. Cell pellets were incubated with RIPA buffer supplemented with Pierce protease inhibitor on ice for 15 minutes. The resulting cell suspension was centrifuged at 11,000 RPM for 15 minutes and protein supernatant recovered. Protein quantification was performed using Bio-Rad's Bradford Protein Assay (5000001). Protein lysates were boiled at 95° C. for 5 minutes in 1× Laemmli Buffer (1610747) supplemented with 355 mM 2-mercaptoethanol. Denatured proteins were resolved by SDS-PAGE using 4-15% polyacrylamide gels and transferred onto 0.45 μm Low Fluorescence PVDF membranes. Membranes were blocked in 5% non-fat dry milk/TBS-T at room temperature for 1 h. Primary antibody incubations were performed according to supplier recommendations. Near InfraRed (NIR) antibodies were used to detect protein on the membranes using the LiCOR Odyssey (CLX-2687) imaging system. Results are shown in Table 9.

TABLE 9
MV411 Biological data

| | SMARCA2 | SMARCA4 |
|---|---|---|
| ADC | % Degradation | % Degradation |
| 1.38B | C | B |

In Table 9, an “A” denotes a % Degradation <50%; a “B” denotes a % Degradation value of 50%<% Degradation ≤75%; a “C” denotes a % Degradation >75%.

### In-Cell Western Blot for SMARCA2/4 Degradation in CL40 and HCT116 Cell Lines

Western blots for SMARCA2/4 degradation in CL40 (CEACAM5 positive) and HCT116 (CEACAM5 negative) cells lines were obtained using analogous procedures as described above for the in-cell western blot in LNCAP and PC3 cell lines. The results are shown in Tables 10 and 11.

TABLE 10
CL40 Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.61A | C | C | C | C |
| 1.61C | A | A | A | A |

TABLE 11
HCT116 Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.61A | A | C | A | C |
| 1.61C | A | C | A | C |

In Tables 10 and 11, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀ <500 ng/mL; a “D” denotes a DC₅₀ value of <50 ng/mL.

In Tables 10 and 11, an “A” denotes a Dₘₐₓ<50%; a “B” denotes a Dₘₐₓ value of 50%<Dₘₐₓ≤75%; a “C” denotes a Dₘₐₓ>75%.

### In-Cell Western Blot for SMARCA2/4 Degradation in HCC1954 and A549 Cell Lines

Western blots for SMARCA2/4 degradation in HCC1954 (TROP2 positive) and A549 (TROP2 negative) cells lines were obtained using analogous procedures as described above for the in-cell western blot in LNCAP and PC3 cell lines. The results are shown in Tables 12 and 13.

TABLE 12
HCC1954 Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.61B | D | C | C | C |
| 1.61C | A | A | A | B |

TABLE 13
A549 Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.61B | A | B | — | — |
| 1.61C | A | B | — | — |

In Tables 12 and 13, an “A” denotes a DC₅₀ value of >5000 ng/mL; a “B” denotes a DC₅₀ value of 500 ng/mL<DC₅₀<5000 ng/mL; a “C” denotes a DC₅₀ value of 50 ng/mL<IC₅₀ <500 ng/mL; a “D” denotes a DC₅₀ value of <50 ng/mL.

In Tables 12 and 13, an “A” denotes a Dₘₐₓ<50%; a “B” denotes a Dₘₐₓ value of 50%<Dₘₐₓ<75%; a “C” denotes a Dₘₐₓ>75%.

### In-Cell Western Blot for SMARCA2/4 Degradation in HS756T Cell Line

Western blot for SMARCA2/4 degradation in HS756T (c-MET positive) cell line was obtained using analogous procedures as described above for the in-cell western blot in LNCAP and PC3 cell lines. The results are shown in Table 14.

TABLE 14
HS746T Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.61D | D | C | C | C |
| 1.61C | B | C | A | C |

In Table 14, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀<500 ng/mL; a “D” denotes a DC₅₀ value of <50 ng/mL.

In Table 14, an “A” denotes a Dₘₐₓ<50%; a “B” denotes a Dₘₐₓ value of 50%<Dₘₐₓ≤75%; a “C” denotes a Dₘₐₓ>75%.

### In-Cell Western Blot for SMARCA2/4 Degradation in CD123′ Cell Line

Western blot for SMARCA2/4 degradation in MOLM13 (CD123 positive) cell line was obtained using analogous procedures as described above for the in-cell western blot in LNCAP and PC3 cell lines. The results are shown in Table 15.

TABLE 15
MOLM13 Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.10D | D | C | C | C |

In Table 15, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀<500 ng/mL; a “D” denotes a DC₅₀ value of <50 ng/mL.

In Table 15, an “A” denotes a Dₘₐₓ<50%; a “B” denotes a Dₘₐₓ value of 50%<Dₘₐₓ≤75%; a “C” denotes a Dₘₐₓ>75%.

### In-Cell Western Blot for SMARCA2/4 Degradation in CD123⁻ Cell Line

Western blot for SMARCA2/4 degradation in JURKAT (CD123 negative) cell line was obtained using analogous procedures as described above for the in-cell western blot in LNCAP and PC3 cell lines. The results are shown in Table 16.

TABLE 16
JURKAT Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.10D | B | B | B | B |

In Table 16, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀<500 ng/mL; a “D” denotes a DC₅₀ value of <50 ng/mL.

In Table 16, an “A” denotes a Dₘₐₓ<50%; a “B” denotes a Dₘₐₓ value of 50%<Dₘₐₓ≤75%; a “C” denotes a Dₘₐₓ>75%.

### In-Cell Western Blot for SMARCA2/4 Degradation in B7H3⁻ Cell Line

Western blot for SMARCA2/4 degradation in KATOIII (B7H3 negative) cell line was obtained using analogous procedures as described above for the in-cell western blot in LNCAP and PC3 cell lines. The results are shown in Table 17.

TABLE 17
KATOIII Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.61E | C | C | C | C |

In Table 17, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀<500 ng/mL; a “D” denotes a DC₅₀ value of <50 ng/mL.

In Table 17, an “A” denotes a Dₘₐₓ<50%; a “B” denotes a Dₘₐₓ value of 50%<Dₘₐₓ≤75%; a “C” denotes a Dₘₐₓ>75%.

### Western Blot for SMARCA2/4 Degradation in Ba/F3-MPL-CALRins5 Cell Line

Western blot for SMARCA2/4 degradation in Ba/F3-MPL-CALRins5 cell line (Kyinno Biotechnology: Catalog #: KC-3784-DW) was obtained using analogous procedures as described above for the western blot in the MV411 cell line. The results are shown in Table 18.

TABLE 18
Ba/F3 CALR^{ins5} Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.85A | C | C | C | C |
| 1.85B | B | C | B | C |

In Table 18, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀<500 ng/mL; a “D” denotes a DC₅₀ value of <50 ng/mL.

In Table 18, an “A” denotes a Dₘₐₓ<50%; a “B” denotes a Dₘₐₓ value of 50%<Dₘₐₓ≤75%; a “C” denotes a Dₘₐₓ>75%.

### Western Blot for SMARCA2/4 Degradation in Ba/F3-MPL-CALR Cell Line

Western blot for SMARCA2/4 degradation in Ba/F3-MPL-CALR cell line (Kyinno Biotechnology: Catalog #: KC-3914) was obtained using analogous procedures as described above for the western blot in the MV411 cell line. The results are shown in Table 19.

TABLE 19
Ba/F3 CALR^{WT} Biological Data

| | SMARCA2 | SMARCA2 | SMARCA4 | SMARCA4 |
|---|---|---|---|---|
| ADC No. | DC₅₀ | Dₘₐₓ % | DC₅₀ | Dₘₐₓ % |
| 1.85A | B | C | B | C |

In Table 19, an “A” denotes a DC₅₀ value of >5000 ng/mL; a “B” denotes a DC₅₀ value of 500 ng/mL<DC₅₀≤5000 ng/mL; a “C” denotes a DC₅₀ value of 50 ng/mL<IC₅₀<500 ng/mL; a “D” denotes a DC₅₀ value of <50 ng/mL.

In Table 19, an “A” denotes a Dₘₐₓ<50%; a “B” denotes a Dₘₐₓ value of 50%<Dₘₐₓ≤75%; a “C” denotes a Dₘₐₓ>75%.

### Example 30. In Vivo Studies

In vivo studies were performed in a PSMA+ cell line derived xenograft to determine whether anti-PSMA-SMARCA2/4 degrader antibody conjugate 1.38A (“Compound 1”) would demonstrate anti-tumor activity and target engagement, while improving tolerability compared to direct administration of the corresponding payload (“Compound 2”) having the following structure:

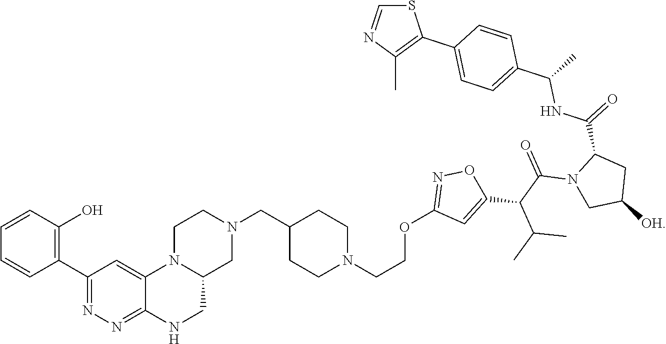 [SMILES: Cc1ncsc1-c1ccc([C@H](C)NC(=O)[C@@H]2C[C@@H](O)CN2C(=O)[C@@H](c2cc(OCCN3CCC(CN4CCN5c6cc(-c7ccccc7O)nnc6NC[C@H]5C4)CC3)no2)C(C)C)cc1]

Efficacy and single dose pharmacodynamic studies with Compound 1 and Compound 2 were performed in a PSMA+ prostate cancer CDX (LNCaP). In-life evaluation was conducted using 6-8-week-old male CB17 SCID mice that were subcutaneously inoculated with LNCaP tumor cells mixed 1:1 with Matrigel. Mice were castrated 3 days prior to randomization, which was done when tumors for efficacy evaluation would reach 100 mm³-400 mm³. Mice were randomized into vehicle or treatment groups (n=6 mice/group) to administer Compound 1 (20 mg/kg, i.v. infusion, every week “QW”) or Compound 2 (10 mg/kg, s.c., every 3 days “Q3D”) for efficacy studies. For pharmacodynamic studies, mice were randomized into vehicle or treatment groups (n=9 mice/group) to administer Compound 1 (20 mg/kg, i.v. infusion) or Compound 2 (10 mg/kg, s.c.) given as a single dose before collecting tumors 1-5 days later. Tumors were subsequently homogenized, and proteins were extracted before evaluating SMARCA2 and SMARCA4 expression by western blot.

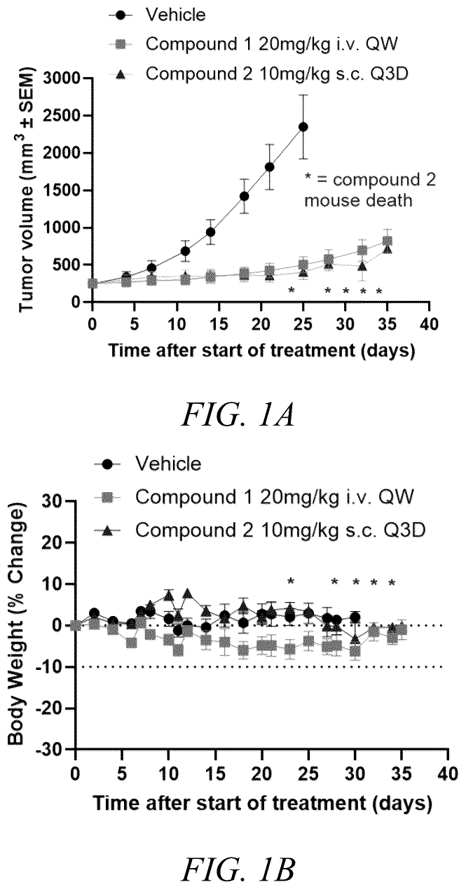

As shown in FIGS. 1A and 1i, Compound 1 resulted in similar tumor growth inhibition (TGI) (TGI=89%, p=0.0014, day 21) as compared to Compound 2 (TGI=93%, p=0.0010, day 21). However, 83.3% of mice treated with Compound 2 died during the study, while mice treated with Compound 1 demonstrated improved tolerability with minimal body weight loss and no mouse deaths.

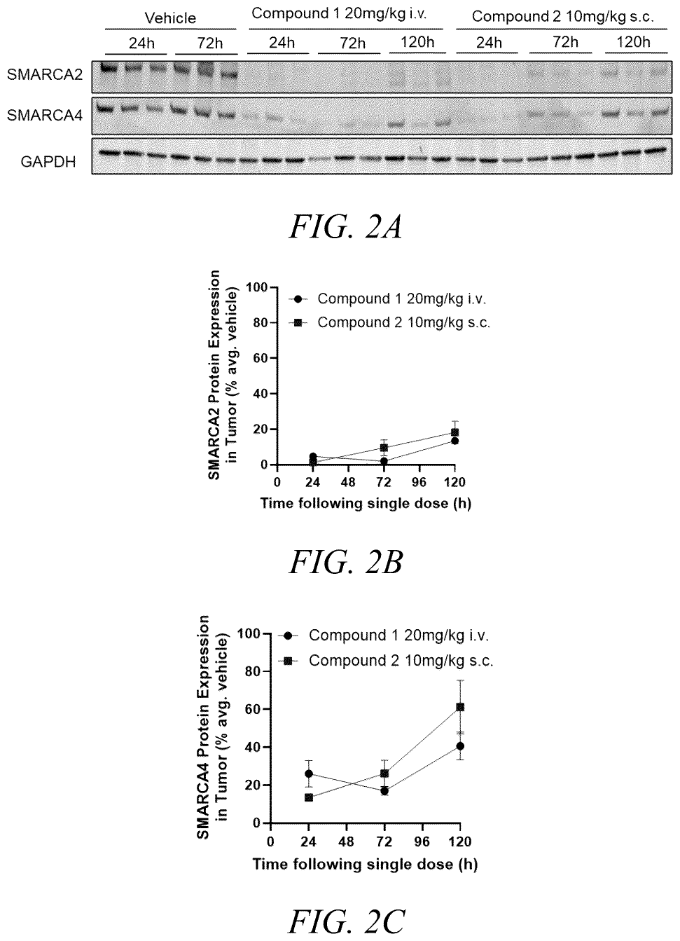

As shown in FIGS. 2A-2C, treatment of mice harboring LNCaP tumors with a single dose of Compound 1 maximally induced degradation of SMARCA2 (98%) and SMARCA4 (83%), which was comparable to that achieved by Compound 2 (SMARCA2=99%, SMARCA4=86%). Greater than 50% degradation of both SMARCA2 and SMARCA4 was observed for longer than 3 days with a single dose of either Compound 1 or Compound 2, while more than 80% degradation of SMARCA2 was achieved for at least 5 days.

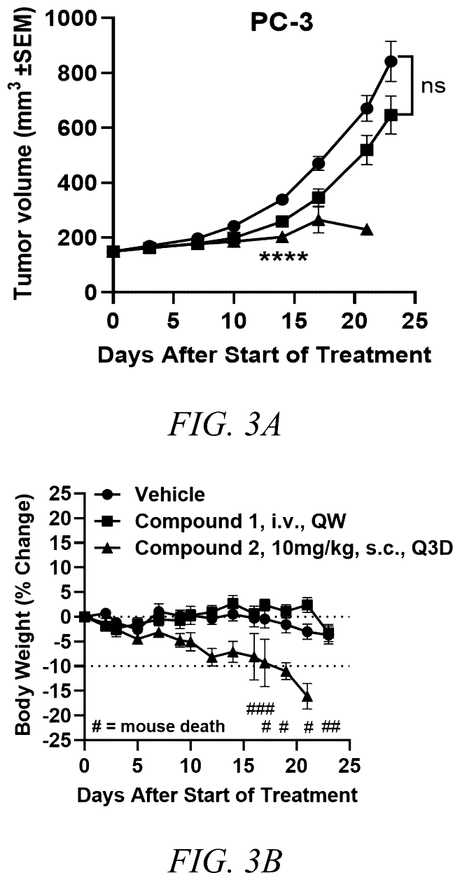

Weekly administration of Compound 1 did not induce significant tumor growth inhibition in PSMA negative PC3 tumors, in comparison to Compound 2 which was efficacious, but caused mouse body weight loss and death, as shown in FIGS. 3A-3B.

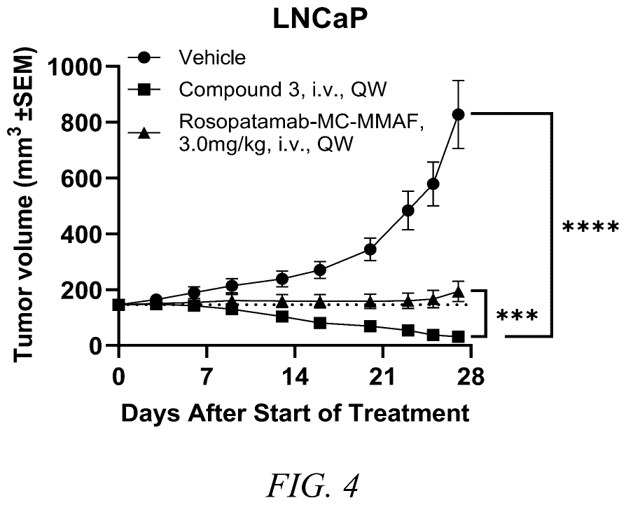

Weekly intravenous administration of anti-PSMA-SMARCA2/4 degrader antibody conjugate 1.38E (“Compound 3”) demonstrated tumor regression and significantly better efficacy compared to a PSMA cytotoxic antibody drug conjugate (rosopatamab-maleimidocaproyl monomethylauristatin F, DAR2) in LNCaP tumors, as shown in FIG. 4 (“****” indicates a p value <0.0001 by Mann-Whitney test or unpaired t-test).

What is claimed is:

1. An Antibody Drug Conjugate compound, or a pharmaceutically acceptable salt thereof, having the structure of: 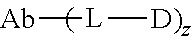
wherein,
Ab is an antibody or an antigen-binding fragment thereof,
L is a linker;
D is a degrader compound of Formula (I):
PTM-ULM (I)
wherein,
PTM is a moiety of Formula IA: 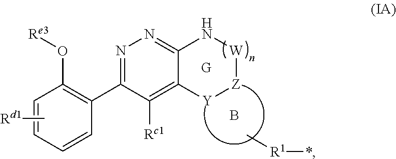
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase; and
subscript z is an integer ranging from 1 to 14.

2. An Antibody Drug Conjugate compound, or a pharmaceutically acceptable salt thereof, of claim 1 having the structure of: 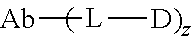
wherein,
Ab is an antibody or an antigen-binding fragment thereof,
D is a degrader compound of Formula (I):
PTM-ULM (I)
wherein,
PTM is a moiety of Formula IA: 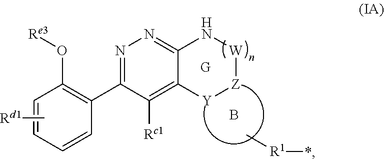
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (II): 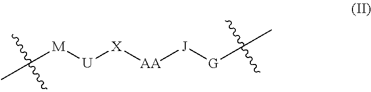
wherein,
M is selected from the group consisting of: 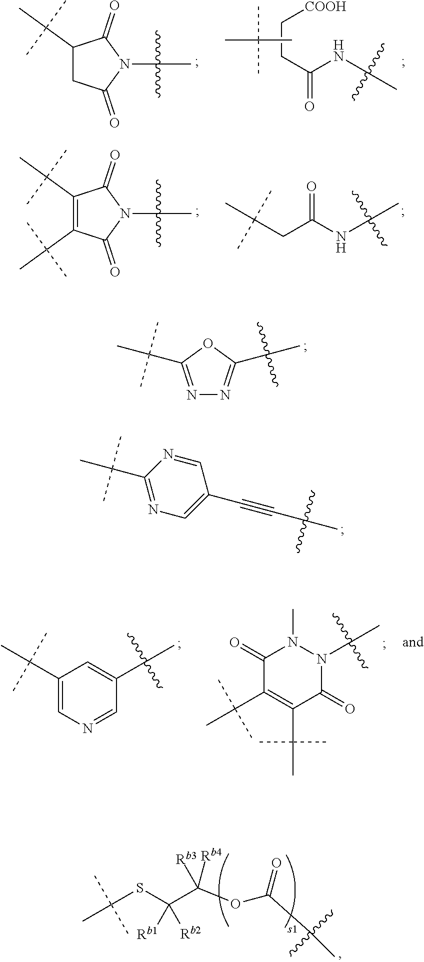
wherein
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1,
wherein the dashed line indicates the point of covalent attachment to the Ab and the wavy line indicates the point of covalent attachment to the remainder of the structure of L;
U is absent or is
—(CH₂)_{b}(R^{v1})ₛₛ(C═O)ᵤ(NH)ᵥ— wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—, wherein R^{x5} is hydrogen or C₁-C₆ alkyl and R^{x6} is hydrogen, C₁-C₆ alkyl, or 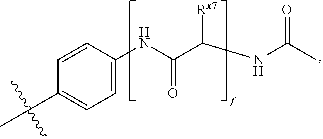
wherein subscript f is an integer ranging from 0 to 4 and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
AA is absent or has the structure of: 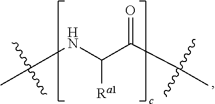
wherein subscript c is an integer ranging from 1 to 12; each R^{a1} is independently —COOH, —NH₂, or an independently selected side chain of an amino acid;
J is absent, —(R^{j3})N(C(R^{j1})(R^{j2}))ₘ₁—, or has the structure of: 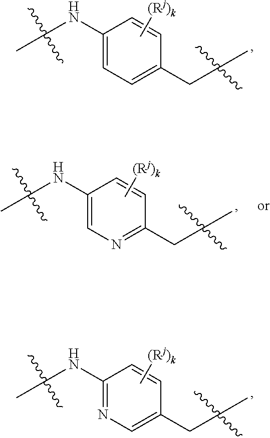
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
G is absent, 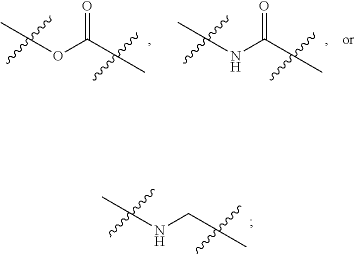
and
wherein the wavy line to M of Formula (II) indicates the point of covalent attachment to Ab and the wavy line to G of Formula (II) indicates the point of covalent attachment to D,
wherein subscript z is an integer ranging from 1 to 14.

3. An Antibody Drug Conjugate compound, or a pharmaceutically acceptable salt thereof, of claim 1 having the structure of: 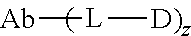
wherein,
Ab is an antibody or an antigen-binding fragment thereof;
D is a degrader compound of Formula (I):
PTM-ULM (I)
wherein,
PTM is a moiety of Formula IA: 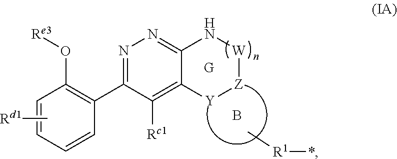
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase;
L is a linker of Formula (IIa): 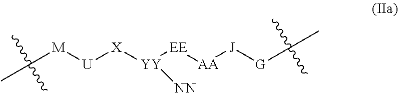
wherein,
M is selected from the group consisting of: 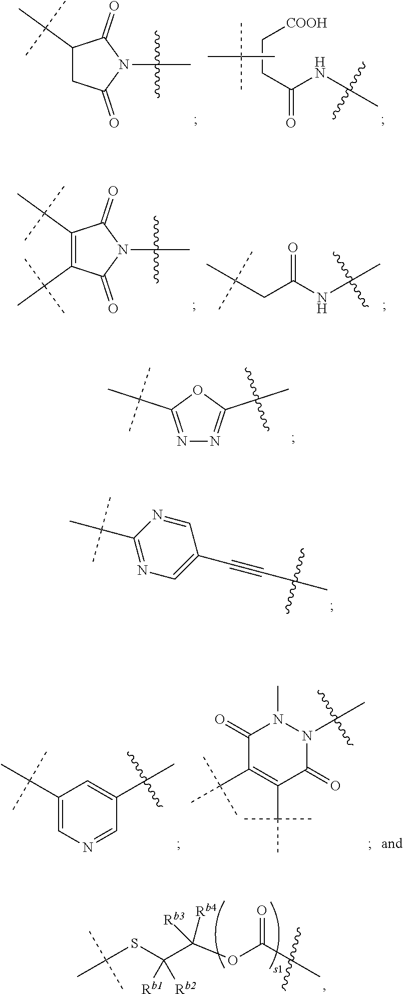
wherein
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1,
wherein the dashed line indicates the point of covalent attachment to the Ab and the wavy line indicates the point of covalent attachment to the remainder of the structure of L′;
U is absent or is —(CH₂)_{b}(R^{v1})ₛₛ(C═O)ᵤ(NH)ᵥ— wherein subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—, wherein R^{x5} is hydrogen or C₁-C₆ alkyl and R^{x6} is hydrogen, C₁-C₆ alkyl, or 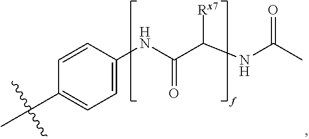
wherein subscript f is an integer ranging from 0 to 4 and each R^{x7} is independently hydrogen, —COOH, —NH₂, C₁-C₆ alkyl, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of: 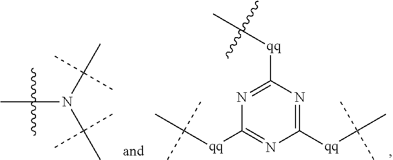
wherein
each qq is independently —N(R^{y1})— or —O—, wherein R^{y1} is hydrogen or C₁-C₆ alkyl,
one dashed line indicates the point of covalent attachment to EE when EE is present, or to AA when EE is absent, or to J when AA and EE are absent, or to G when AA, EE, and J are absent, and the other dashed line indicates the point of covalent attachment to NN, wherein the wavy line indicates the point of covalent attachment to X, when X is present, or to U, when X is absent, or to M, when X and U are absent;
NN is —(CH₂CH₂O)_{k′}CH₂CH₂C(O)—Rⁿⁿ¹-, wherein subscript k′ is an integer ranging from 0 to 16 and Rⁿⁿ¹ is hydrogen, C₁-C₆ alkyl, or —OH;
EE is absent or —(CH₂CH₂O)_{x′}CH₂CH₂C(O)—, wherein subscript x′ is an integer ranging from 0 to 16;
AA is absent or has the structure of: 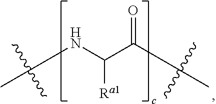
wherein subscript c is an integer ranging from 1 to 12; each R^{a1} is independently —COOH, —NH₂, or an independently selected side chain of an amino acid;
J is independently absent, —(R^{j3})N(C(R^{j1})(R^{j2}))ₘ₁—, or has the structure of: 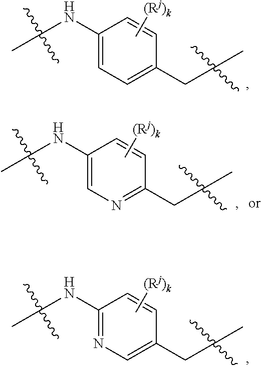
wherein
R^{j3} is hydrogen, C₁-C₆ alkyl, or C₃-C₆ cycloalkyl;
each R^{j} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
G is absent, 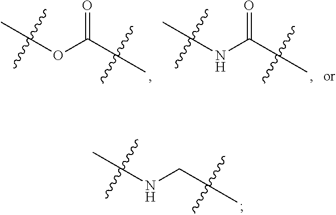
and
wherein the wavy line to M of Formula (IIa) indicates the point of covalent attachment to Ab and the wavy line to G of Formula (IIa) indicates the point of covalent attachment to D.

4. The Antibody Drug Conjugate compound of any one of the preceding claims, or a pharmaceutically acceptable salt thereof, wherein R¹ is a covalent bond.

5. The Antibody Drug Conjugate compound of any one of the preceding claims, or a pharmaceutically acceptable salt thereof, wherein R¹ is a chemical moiety represented by the formula:
-(A)q-,
wherein:
q is an integer from 1 to 14;
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR¹d, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a} (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d} 3-11 membered cycloalkyl, optionally substituted with 0-6 R^{1a} and/or R^{1b} groups, 3-11 membered heterocyclyl optionally substituted with 0-6 R^{1a} and/or R^{1b} groups, aryl optionally substituted with 0-6 R^{1a} and/or R^{1b} groups, and heteroaryl optionally substituted with 0-6 R^{1a} and/or R^{1b} groups,
wherein R^{1a}, R^{1b}, R^{1c}, R^{1d} and R^{1c} are each independently, —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC1-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R groups.

6. The Antibody Drug Conjugate compound of any one of the preceding claims, or a pharmaceutically acceptable salt thereof, wherein ULM binds Von Hippel-Lindau E3 Ubiquitin Ligase.

7. The Antibody Drug Conjugate compound of claim 6, or a pharmaceutically acceptable salt thereof, wherein ULM is a moiety having the Formula ULM-I-VHL-1: 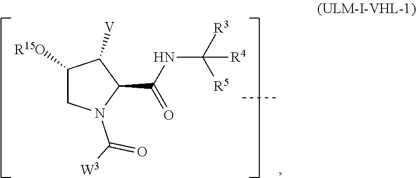
wherein
the dashed line ( [symbol] ) indicates the position of attachment of ULM-I-VHL-1 to R¹;
R¹⁵ is hydrogen or —PO₃H₂;
V is H or F;
R³ is optionally substituted phenyl, optionally substituted napthyl, or an optionally substituted 5-10 membered heteroaryl;
one of R⁴ or R⁵ is H, deuterium, haloalkyl, optionally substituted alkyl, optionally substituted cycloalkyl, optionally substituted heterocycloalkyl, or —COR^{d}v CONR^{e1}R^{e2};
the other of R⁴ or R⁵ is H or deuterium;
or R⁴ and R⁵, together with the carbon atom to which they are both attached, form an optionally substituted 3-5 membered cycloalkyl or heterocyclyl;
W³ is an optionally substituted aryl, optionally substituted heteroaryl, or 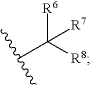
R⁶ and R⁷ are independently H, deuterium, optionally substituted alkyl, optionally substituted cycloalkyl, or optionally substituted haloalkyl,
or R⁶, R⁷, and the carbon atom to which they are attached form an optionally substituted cycloalkyl or optionally substituted heterocyclyl;
R⁸ is an optionally substituted heterocyclyl, optionally substituted heteroaryl, optionally substituted aryl, CONR^{av}R^{bv}, NR^{av}R^{bv}, 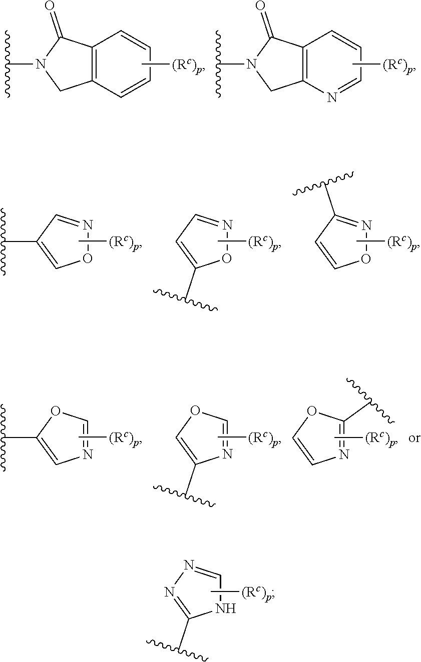
R^{av} is H or optionally substituted alkyl;
R^{bv} is H, —C(O)—* wherein * is a point of attachment to R¹, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{c} is independently H, halo, optionally substituted alkoxy, cyano, optionally substituted alkyl, haloalkyl, or haloalkoxy;
each R^{dv} is independently H, optionally substituted alkyl or NR^{e1}R^{e2};
each R^{e1} and R^{e2} is independently H, deuterium, or optionally substituted alkyl,
or R^{e1} and R^{e2} together with the nitrogen atom to which they are attached form a 4-7 membered heterocyclyl; and
p is 0, 1, 2, 3, or 4.

8. The Antibody Drug Conjugate compound of claim 6 or 7, or a pharmaceutically acceptable salt thereof, wherein ULM-I-VHL-1 is a compound of formula: 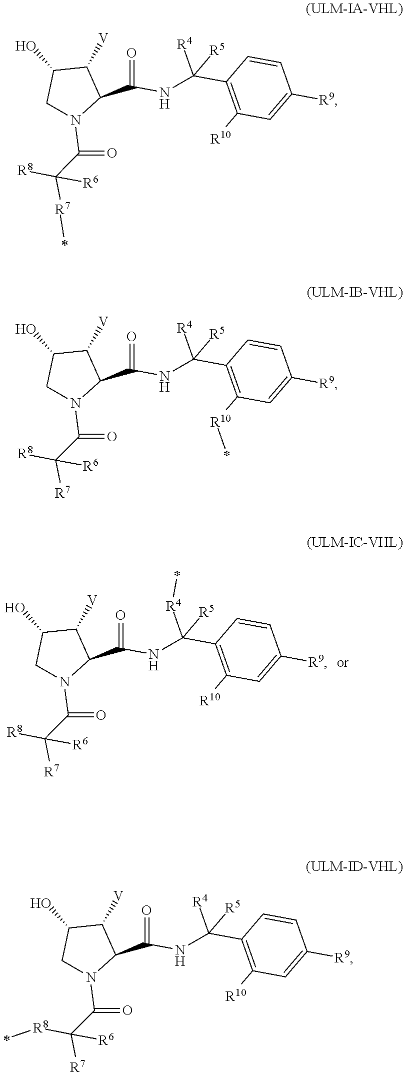
wherein * is a point of attachment of the ULM to R¹.

9. The Antibody Drug Conjugate compound of any one of claims 6 to 8, or a pharmaceutically acceptable salt thereof, wherein D has a structure of: 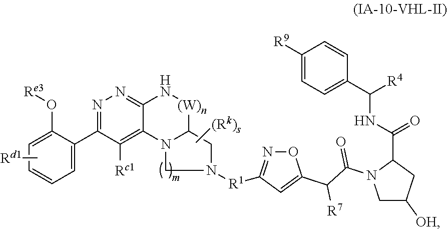
wherein
W is optionally substituted —CH₂—, —C(O)—, —S(O)—, or —S(O)₂—; wherein when n=2 or 3, only one W may be —C(O)—, —S(O)—, or —S(O)₂—;
n=0-3;
m=1-3;
R^{k}=H, deuterium, F, C₁₋₃ alkyl, C₁₋₃ haloalkyl, or C₁₋₄ alkoxyl;
s=0-3;
R^{c1} and R^{d1} are independently H, deuterium, Halo, C₁₋₃ alkyl, C₁₋₃ haloalkyl, or C₁₋₄ alkoxyl;
R^{e3} is H, —C(O)R, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b}groups, —(CR^{1a}R^{1b})₁₋₅, —(CR^{1a}═CR^{1b})—, —(CR^{1a}R^{1b})₁₋₅-A- wherein A is O, S, or NR^{1c}, —(CR^{1a}R^{1b})₁₋₅-A-(CR^{1a}R^{1b})₁₋₅— wherein A is O, S, or NR^{1c}—(CR^{1a}R^{b})₁₋₅-A-(CR^{1a}R^{b})₁₋₅-A- wherein A is O, S, or NR^{1c}, —(CR^{1a}R^{1b})₁₋₅—(CR^{1a}=CR^{1b})—(CR^{1a}R^{1b})₁₋₅—, —(CR^{1a}R^{1b})l5-(CR^{1a}=CR^{1b})—(CR^{1a}R^{1b})₁₋₅-A- wherein A is O, S, or NR^{1c}—(CR^{1a}R^{b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b} groups)-, -(3-11 membered cycloalkyl optionally substituted with 0-6 R^{1a} and/or R^{1b} groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b}groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-(3-11 membered heterocyclyl optionally substituted with 0-6 R^{1a} and/or R^{1b} groups)-A- wherein A is O, S, or NR^{1c}—(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 0-6 R^{1a} and/or R^{1b}groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}—(CR^{1a}R^{1b})₁₋₅-A-(CR^{1a}R^{1b})₁₋₅-A- wherein A is O, S, or NR^{1c}—(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}—(CR^{1a}R^{1b})₁₋₅-A-(CO) wherein A is O, S, or NR^{1c}—(CR^{1a}R^{1b})₁₋₅—(CR^{1a}═CR^{1b})—(CR^{1a}R^{1b})₁₋₅-A-(CO)— wherein A is O, S, or NR^{1c}—(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b} groups)- wherein A is O, S, or NR^{1c}—(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}-(3-11 membered cycloalkyl optionally substituted with 0-6 R^{1a} and/or R^{1b} groups)-(CR^{1a}R^{1b})₁₋₅₋, or -(3-11 membered heterocyclyl optionally substituted with 0-6 R^{1a} and/or R^{1b} groups)-(CR^{1a}R^{1b})₁₋₅; or R^{j} is -(3-11 membered heterocyclyl optionally substituted with 0-6 R^{1a} and/or R^{1b} groups)-(CO)—(CR^{1a}R^{1b})₁₋₅-A- wherein A is O, S, or NR^{1c}; -(3-11 membered heterocyclyl optionally substituted with 0-6 R^{1a} and/or R^{1b} groups)-(CO)-A-(CR^{1a}R^{1b})₁₋₅— wherein A is O, S, or NR^{1c}; -A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}; -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-A-(CR^{1a}R^{1b})₁₋₅-A- wherein each A is independently O, S, or NR^{1c}; -(heteroaryl optionally substituted with 0-4 R^{1a} and/or R^{1b} groups)-A-(CR^{1a}R^{1b})₁₋₅— wherein A is O, S, or NR^{1c}; -(3-11 membered heterocyclyl optionally substituted with 0-6 R^{1a} and/or R^{1b}groups)-(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)—(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or —(CO)-(3-11 membered cycloalkyl optionally substituted with 0-6 R^{1a} and/or R^{1b} groups)-(CR^{1a}R^{1b})₁₋₅-A- wherein A is O, S, or NR^{1c};
R⁴ is H, optionally substituted alkyl, optionally substituted C₁-C₆alkyl, or —CH₃;
R⁷ is optionally substituted alkyl, preferably optionally substituted C₁-C₆alkyl, and more preferably C₁-C₆alkyl; and
R⁹ is H, deuterium, halo, —CN, —OH, —NO₂, —NR^{e1}R^{e2}, —OR^{e1}, —CONR^{e1}R^{e2}, —NR^{e1}COR^{e2} —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R^{1a}, R^{1b}, R^{1c}, and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{a1}° groups; and
each R^{e1} and R^{e2} is independently H, deuterium, or optionally substituted alkyl, or R^{e1} and R^{e2} together with the nitrogen atom to which they are attached form a 4-7 membered heterocyclyl.

10. The Antibody Drug Conjugate compound of any one of claims 6 to 9, or a pharmaceutically acceptable salt thereof, wherein D has a structure of: 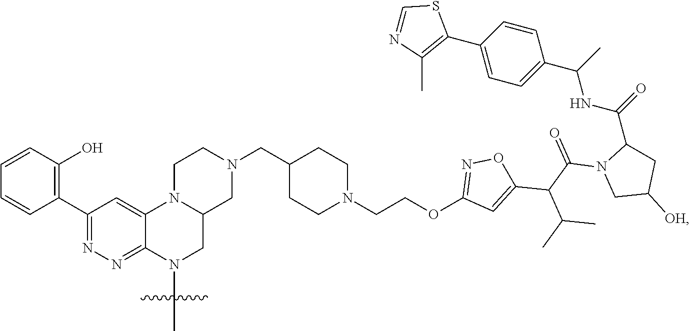 [SMILES: *N1CC2CN(CC3CCN(CCOc4cc(C(C(=O)N5CC(O)CC5C(=O)NC(C)c5ccc(-c6scnc6C)cc5)C(C)C)on4)CC3)CCN2c2cc(-c3ccccc3O)nnc21]
wherein the wavy line indicates the point of covalent attachment to L.

11. The Antibody Drug Conjugate compound of any one of claims 6 to 9, or a pharmaceutically acceptable salt thereof, wherein D has a structure of 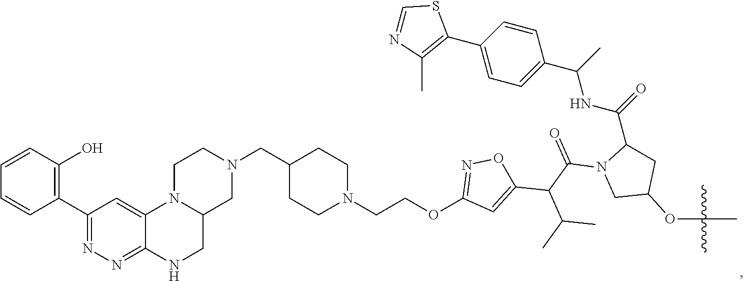 [SMILES: *OC1CC(C(=O)NC(C)c2ccc(-c3scnc3C)cc2)N(C(=O)C(c2cc(OCCN3CCC(CN4CCN5c6cc(-c7ccccc7O)nnc6NCC5C4)CC3)no2)C(C)C)C1]
wherein the wavy line indicates the point of covalent attachment to L.

12. The Antibody Drug Conjugate compound of any one of claims 6 to 9, or a pharmaceutically acceptable salt thereof, wherein D has a structure of 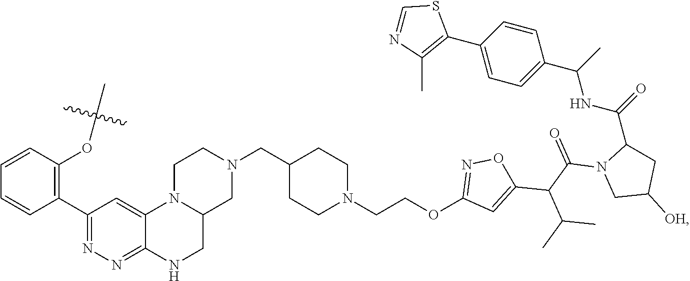 [SMILES: *Oc1ccccc1-c1cc2c(nn1)NCC1CN(CC3CCN(CCOc4cc(C(C(=O)N5CC(O)CC5C(=O)NC(C)c5ccc(-c6scnc6C)cc5)C(C)C)on4)CC3)CCN21]
wherein the wavy line indicates the point of covalent attachment to L.

13. The Antibody Drug Conjugate compound of claim 6 or 7, or a pharmaceutically acceptable salt thereof, wherein D has a structure of: 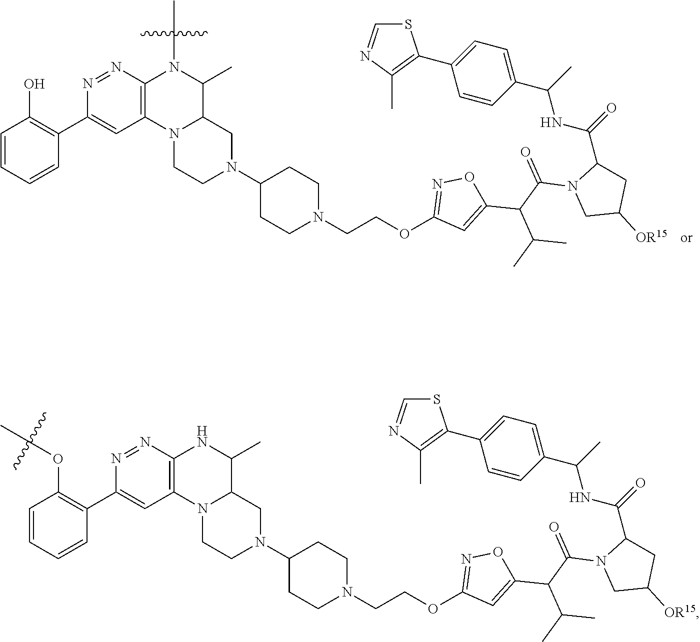
wherein R¹⁵ is hydrogen or —PO₃H₂and the wavy line indicates the point of covalent attachment to L.

14. The Antibody Drug Conjugate compound of claim 13, or a pharmaceutically acceptable salt thereof, wherein D has a structure of:

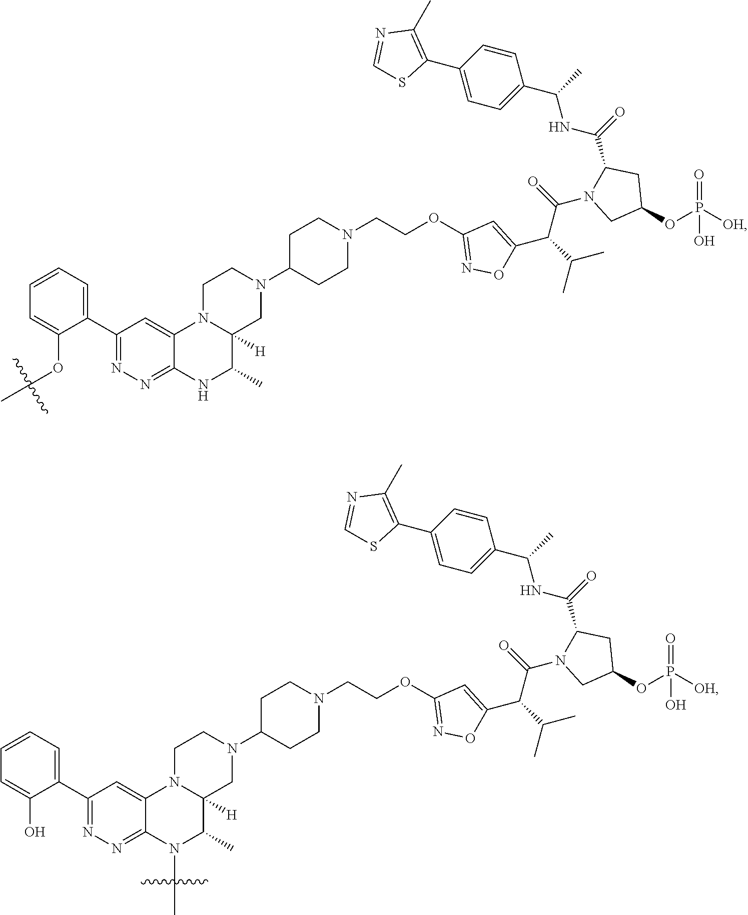 [SMILES: *Oc1ccccc1-c1cc2c(nn1)N[C@@H](C)[C@@H]1CN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)CCN21 ; *N1c2nnc(-c3ccccc3O)cc2N2CCN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)C[C@H]2[C@@H]1C]

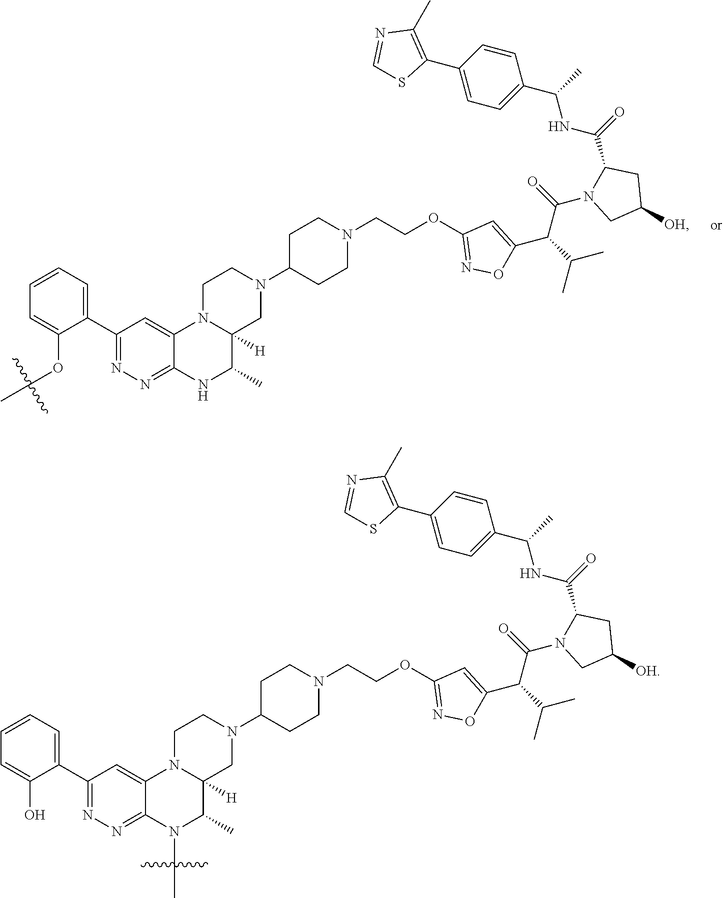 [SMILES: *Oc1ccccc1-c1cc2c(nn1)N[C@@H](C)[C@@H]1CN(C3CCN(CCOc4cc([C@H](C(=O)N5C[C@H](O)C[C@H]5C(=O)N[C@@H](C)c5ccc(-c6scnc6C)cc5)C(C)C)on4)CC3)CCN21 ; *N1c2nnc(-c3ccccc3O)cc2N2CCN(C3CCN(CCOc4cc([C@H](C(=O)N5C[C@H](O)C[C@H]5C(=O)N[C@@H](C)c5ccc(-c6scnc6C)cc5)C(C)C)on4)CC3)C[C@H]2[C@@H]1C]

15. The Antibody Drug Conjugate compound of any one of claims 1 to 14, or a pharmaceutically acceptable salt thereof, wherein ULM binds Cereblon E3 Ubiquitin Ligase.

16. The Antibody Drug Conjugate compound of claim 15, or a pharmaceutically acceptable salt thereof, wherein ULM is a moiety having the Formula ULM-II-CRBN: 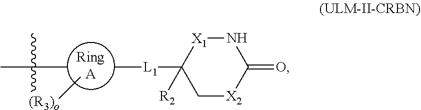
wherein [symbol]
is a point of attachment to PTM;
Ring A is a monocyclic, bicyclic or tricyclic aryl, heteroaryl or heterocycle group,
L₁ is a bond, —O—, —S—, —NR^{a}—, —C(R^{a})₂—, or —C(O)NR^{a}—;
X₁ is a bond, —C(O)—, —C(S)—, —CH₂—, —CHCF₃—, SO₂—, —S(O), P(O)R^{b}—or —P(O)OR^{b}—;
X₂ is —C(R^{a})₂—, —NR^{a}— or —S—;
R₂ is H, deuterium, optionally substituted C₁₋₄ alkyl, C₁₋₄ alkoxyl, C₁₋₄ haloalkyl, —CN, —OR^{a}, —OR^{b} or —SR^{b};
each R₃ is independently H, deuterium, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{e2}R^{d} —NR^{a}R^{c2}, —C(O)R^{b}, —OC(O)R^{a}, —C(O)OR^{a}, —C(O)NR^{e2}R^{d}, —S(O)R^{b}, —S(O)₂NR^{e2}R^{d}, —S(O)(═NR^{b})R^{b}, —SF₅, —P(O)R^{b}R^{b}, —P(O)(OR^{b})(OR^{b}), —B(OR^{d})(OR^{c2}) or —S(O)₂R^{b};
each R^{a} is independently H, deuterium, —C(O)R^{b}, —C(O)OR^{c2}, —C(O)NR^{e2}R^{d}, —C(═NR^{b})NR^{b}R^{c2}, —C(═NOR^{b})NR^{b}R^{c2}, —C(═NCN)NR^{b}R^{c2}, —P(OR^{c2})₂, —P(O)R^{c2}R^{b}, —P(O)OR^{c2}OR^{b}, —S(O)R^{b}, —S(O)NR^{c2}R^{d}, —S(O)₂R^{b}, —S(O)₂NR^{c2}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, deuterium, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c2} or R^{d} is independently H, deuterium, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c2} and R^{d}, together with the atom to which they are both attached, form a monocyclic or multicyclic heterocycloalkyl, or a monocyclic or multicyclic heterocyclo-alkenyl group; and
o is 1, 2, 3, 4, or 5.

17. The Antibody Drug Conjugate compound of claim 15 or 16, or a pharmaceutically acceptable salt thereof, wherein ULM-II-CRBN is a compound of formula: 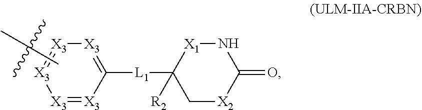
wherein each X₃ is independently N, N-oxide or CR₃ and at least one X₃ is N or N-oxide;
wherein [symbol] is a point of attachment to PTM; or 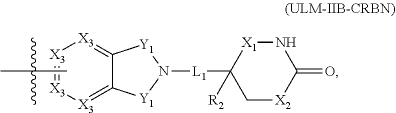
wherein each X₃ is independently N, N-oxide or CR₃;
wherein each Y₁ is independently —C(O)— or —C(R^{a})₂—and at least one Y₁ is —C(O)—; and
wherein [symbol] is a point of attachment to PTM; or 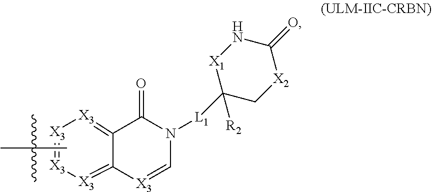
wherein each X₃ is independently N, N-oxide or CR₃ and wherein [symbol] is a point of attachment to PTM; or 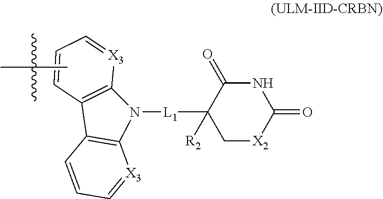
wherein each X₃ is independently N, N-oxide or CR₃ and wherein [symbol] is a point of attachment to PTM.

18. The Antibody Drug Conjugate compound of any one of claims 15 to 17, or a pharmaceutically acceptable salt thereof, wherein D has a structure of: 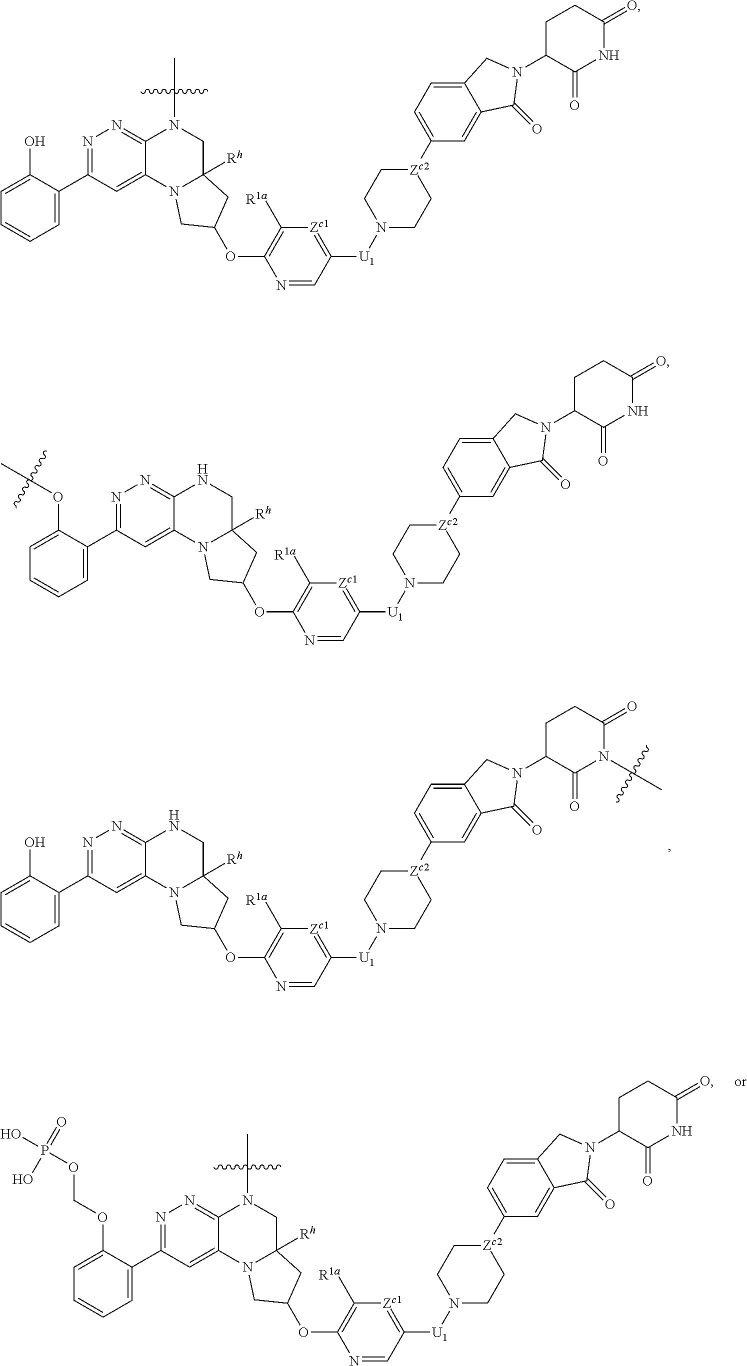 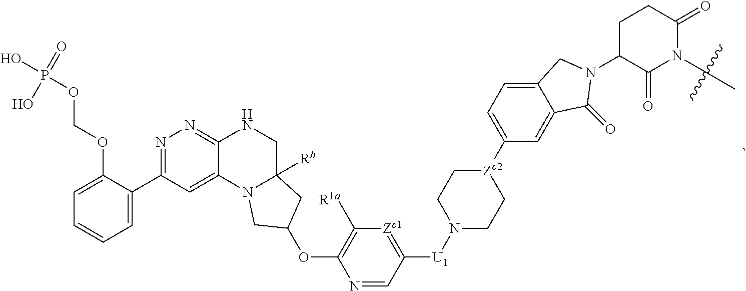
wherein
the wavy line indicates the point of covalent attachment to L;
R^{h} is C₁₋₃ alkyl;
R^{1a} is hydrogen, C₁₋₃ alkyl, or halogen;
Z^{c1} and Z^{c2} are each independently CH or N; and
U₁ is —CH₂— or —C(O)—.

19. The Antibody Drug Conjugate compound of claim 18, or a pharmaceutically acceptable salt thereof, wherein D has a structure of: 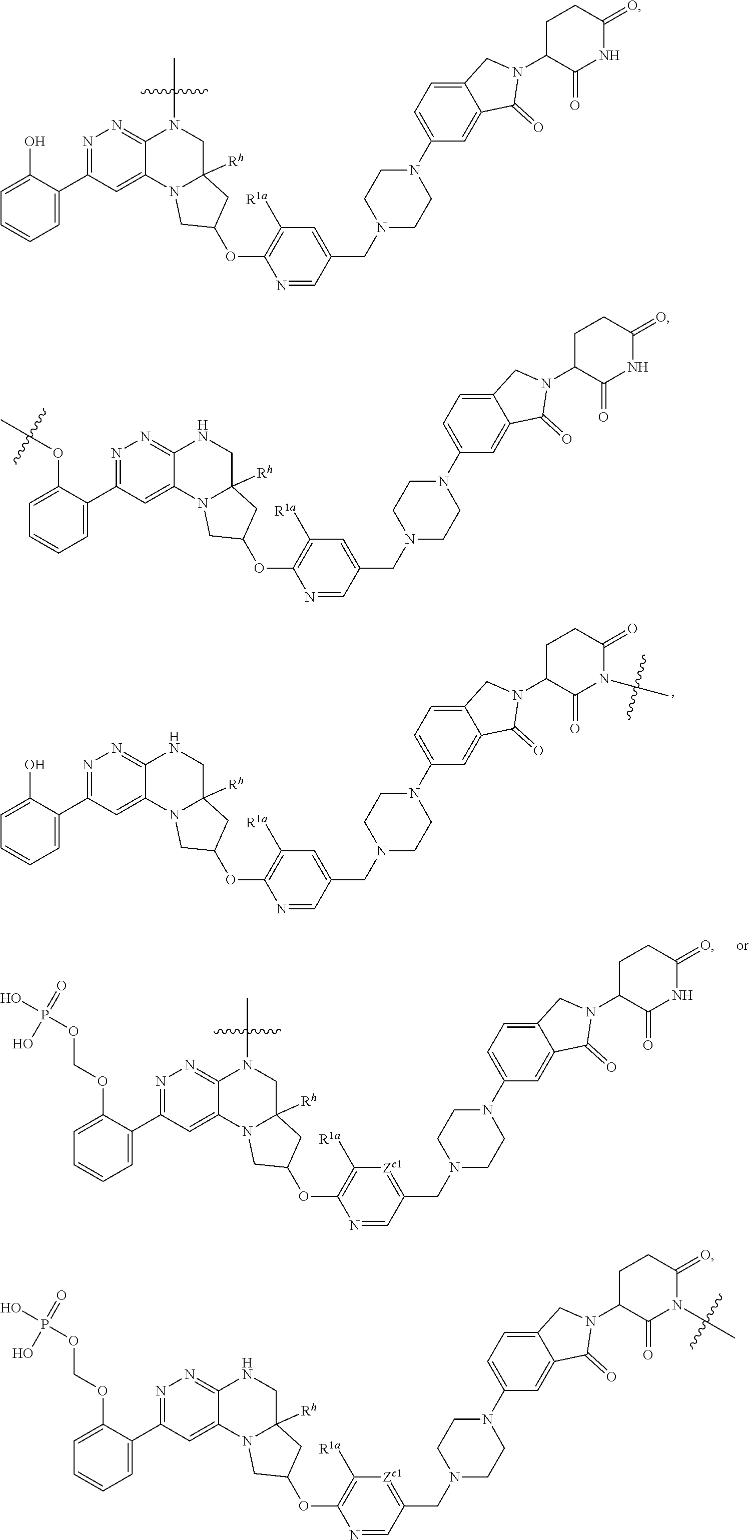
wherein
the wavy line indicates the point of covalent attachment to L;
R^{h} is methyl or ethyl; and
R^{1a} is hydrogen, methyl, or fluorine.

20. The Antibody Drug Conjugate compound of claim 19, or a pharmaceutically acceptable salt thereof, wherein D has a structure of: 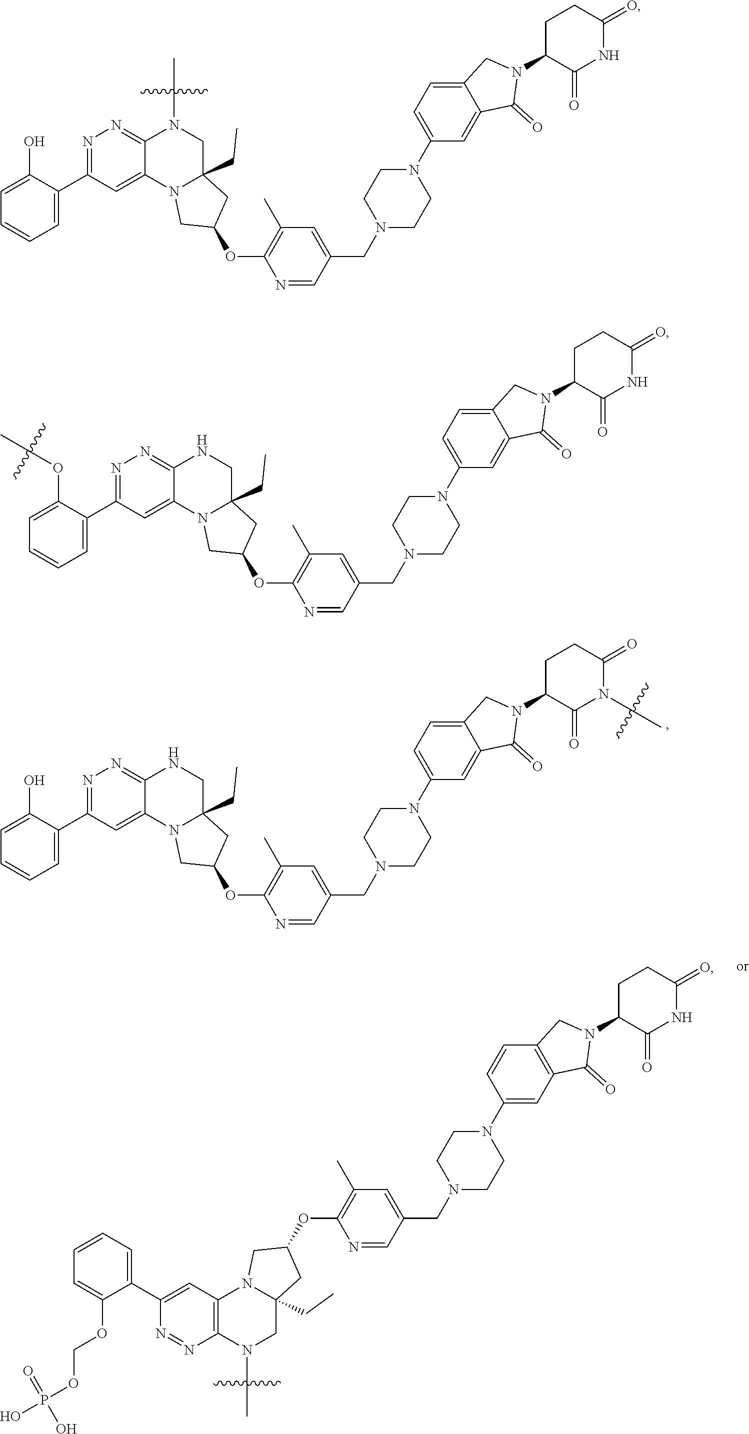 [SMILES: *N1C[C@]2(CC)C[C@@H](Oc3ncc(CN4CCN(c5ccc6c(c5)C(=O)N([C@H]5CCC(=O)NC5=O)C6)CC4)cc3C)CN2c2cc(-c3ccccc3O)nnc21 ; *Oc1ccccc1-c1cc2c(nn1)NC[C@]1(CC)C[C@@H](Oc3ncc(CN4CCN(c5ccc6c(c5)C(=O)N([C@H]5CCC(=O)NC5=O)C6)CC4)cc3C)CN21 ; *N1C(=O)CC[C@H](N2Cc3ccc(N4CCN(Cc5cnc(O[C@H]6CN7c8cc(-c9ccccc9O)nnc8NC[C@]7(CC)C6)c(C)c5)CC4)cc3C2=O)C1=O ; *N1C[C@]2(CC)C[C@@H](Oc3ncc(CN4CCN(c5ccc6c(c5)C(=O)N([C@H]5CCC(=O)NC5=O)C6)CC4)cc3C)CN2c2cc(-c3ccccc3OCOP(=O)(O)O)nnc21 ; "or"] 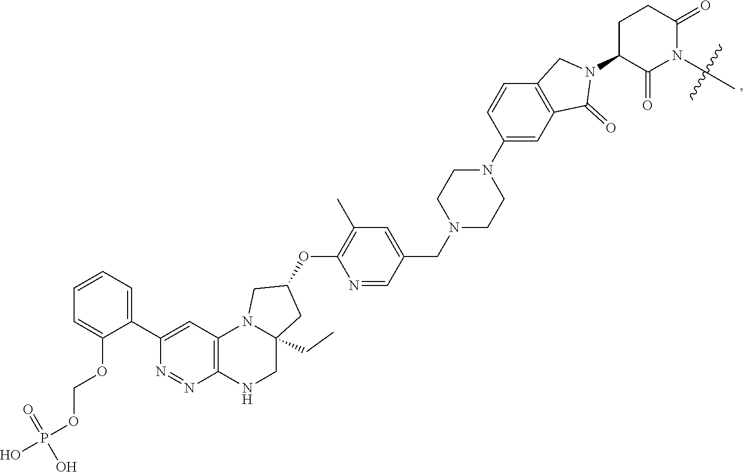 [SMILES: *N1C(=O)CC[C@H](N2Cc3ccc(N4CCN(Cc5cnc(O[C@H]6CN7c8cc(-c9ccccc9OCOP(=O)(O)O)nnc8NC[C@]7(CC)C6)c(C)c5)CC4)cc3C2=O)C1=O]
wherein the wavy line indicates the point of attachment to L.

21. The Antibody Drug Conjugate compound of any one of the preceding claims, or a pharmaceutically acceptable salt thereof, having the formula of:

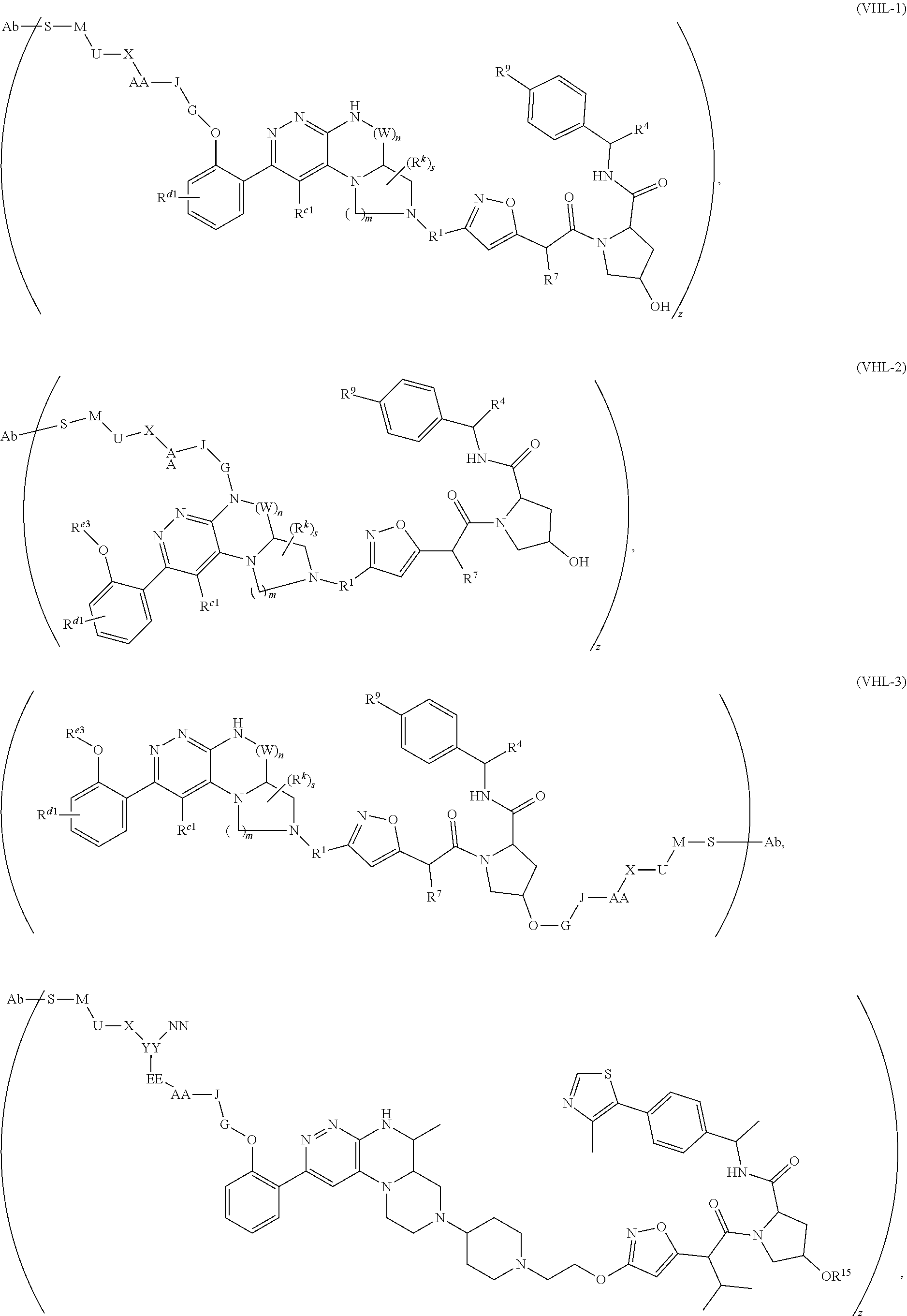

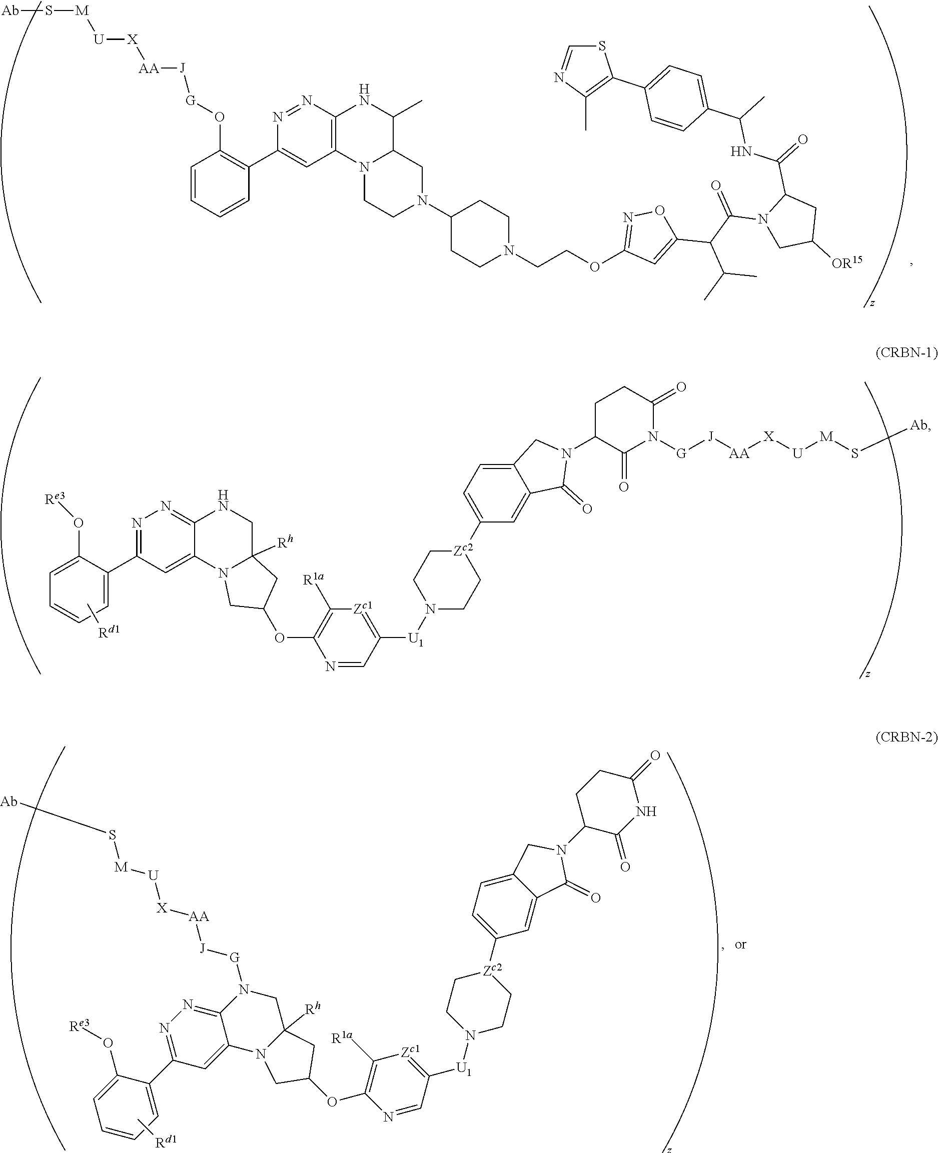

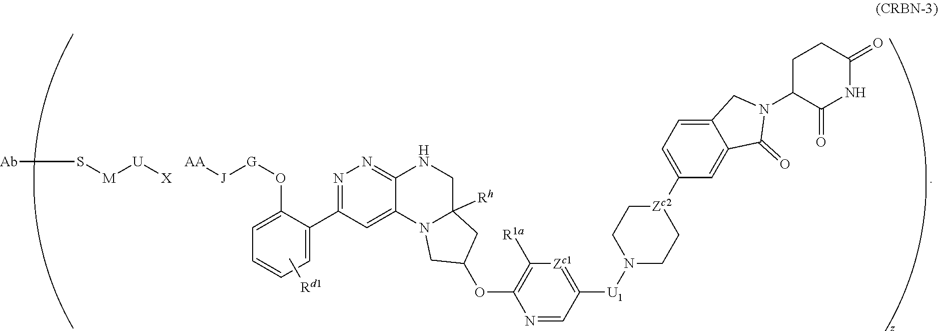

22. The Antibody Drug Conjugate compound of any one of the preceding claims, or a pharmaceutically acceptable salt thereof,
wherein
AA is present;
subscript c is an integer ranging between 1 and 12; and
each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine.

23. The Antibody Drug Conjugate compound of claim 21 or 22, or a pharmaceutically acceptable salt thereof, represented by the structure of: 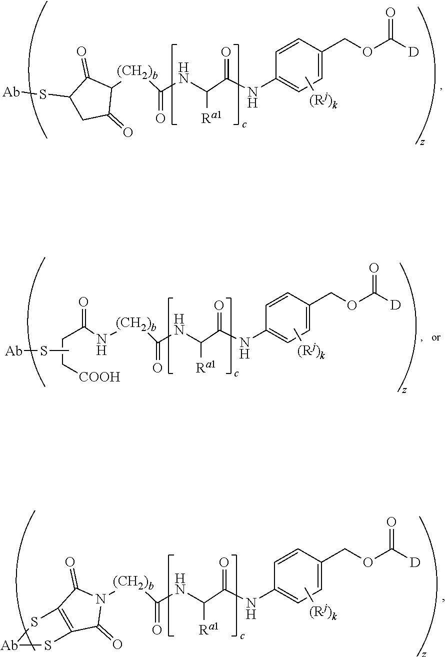
wherein
S is a sulfur atom of the Ab;
subscript b is an integer ranging from 1 to 5;
subscript c is an integer ranging from 1 to 5;
each R^{a1} is independently the side chain of valine, glutamic acid, tryptophan, glycine, citrulline, lysine, alanine, or phenylalanine;
each R^{j} is independently fluorine, chlorine, methyl, or —C(O)NHCH₃; and
subscript k is an integer ranging from 0 to 4.

24. The Antibody Drug Conjugate compound of claim 22, or a pharmaceutically acceptable salt thereof, represented by the structure of: 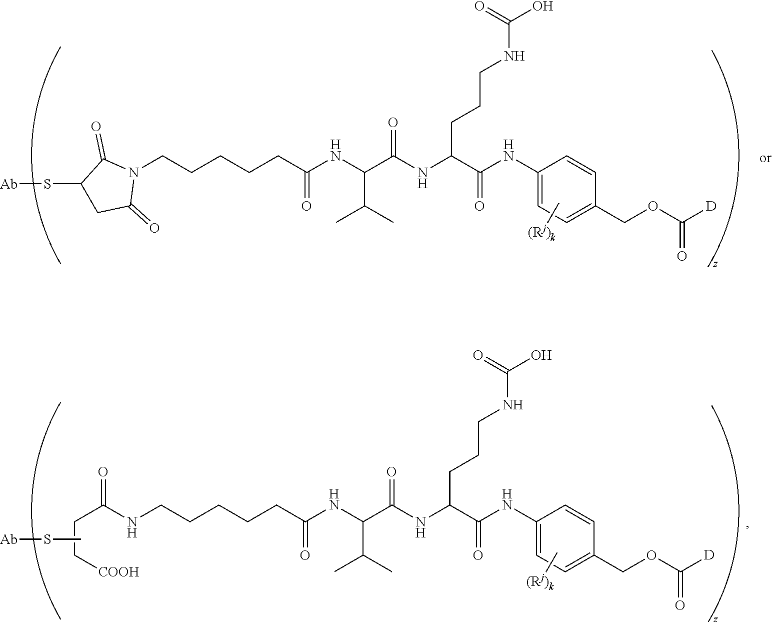
wherein R^{j} is methyl, —F, —Cl, or —C(O)NHCH₃, and subscript k is 0 or 1.

25. The Antibody Drug Conjugate compound of claim 22, or a pharmaceutically acceptable salt thereof, represented by the structure of:

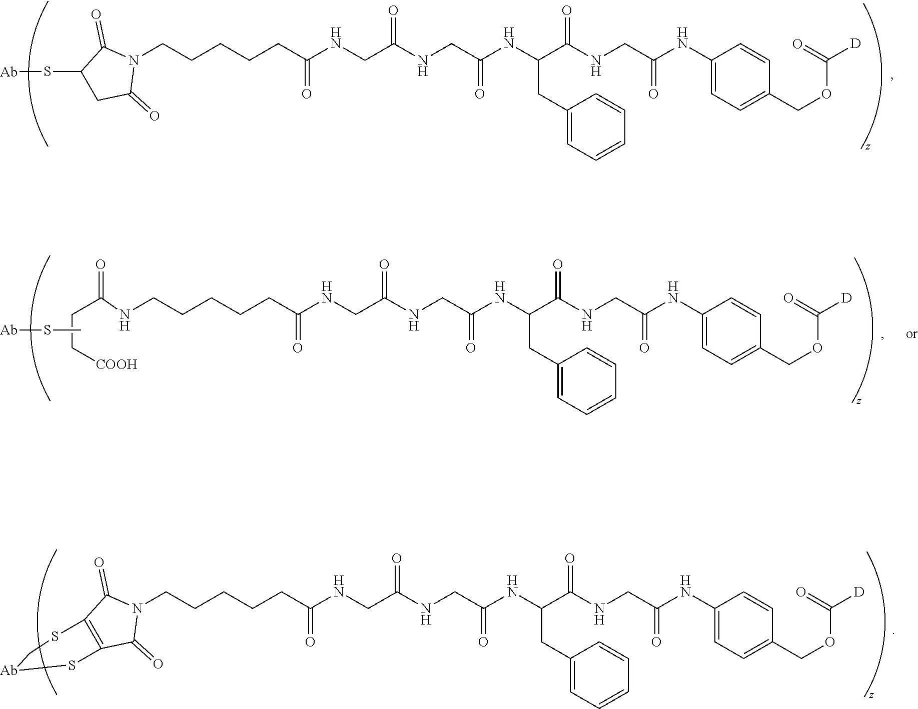

26. The Antibody Drug Conjugate compound of claim 25, or a pharmaceutically acceptable salt thereof, represented by the structure of:

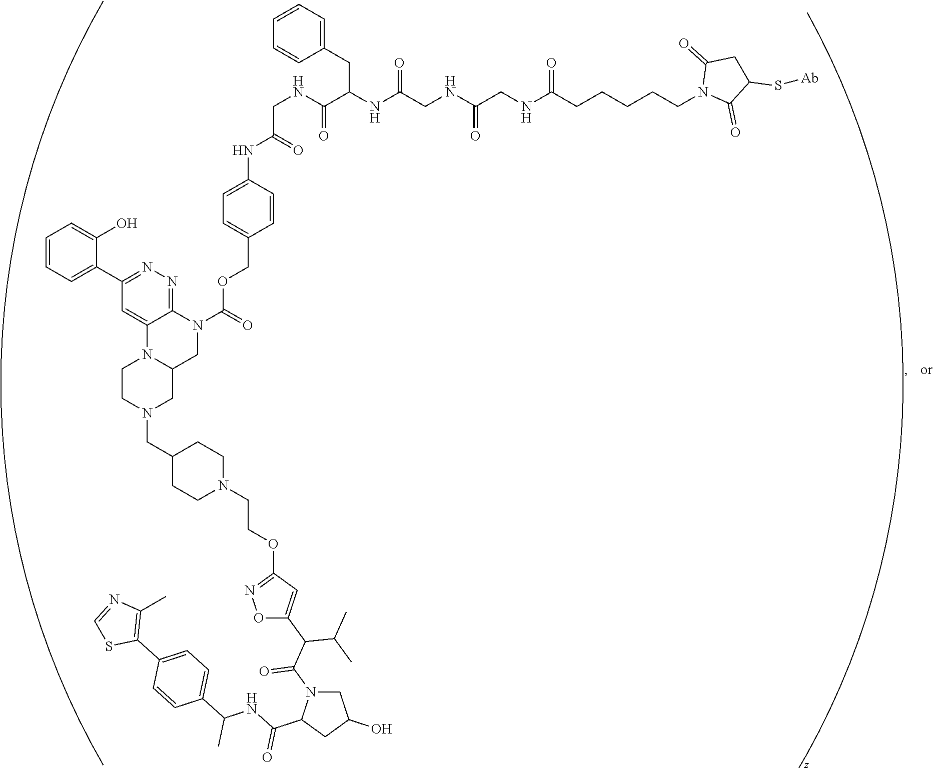

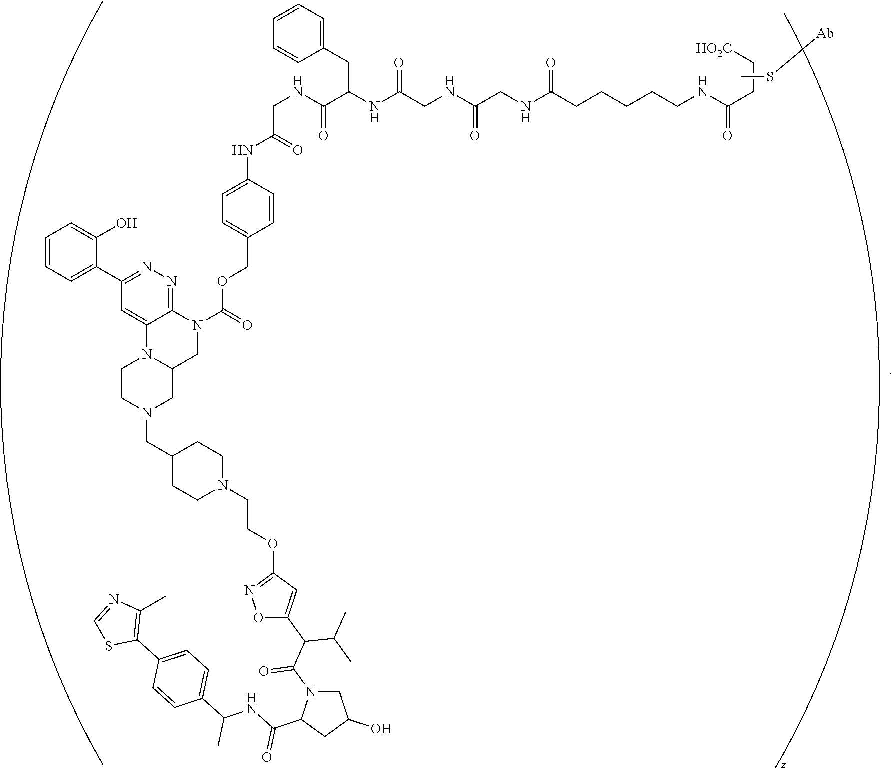

27. The Antibody Drug Conjugate compound of claim 21 or 22, or a pharmaceutically acceptable salt thereof, represented by the structure of: 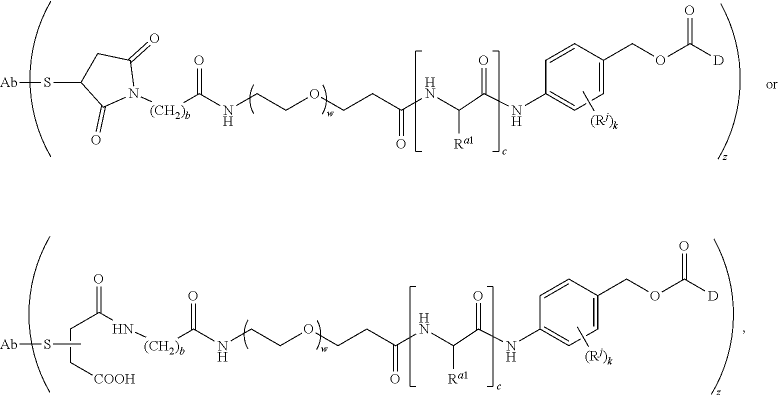
wherein
S is a sulfur atom of the Ab;
subscript b is an integer ranging from 1 to 5;
subscript w is an integer ranging from 1 to 8;
subscript c is an integer ranging from 1 to 3;
each R^{a1} is independently the side chain of valine, glutamic acid, tryptophan, glycine, citrulline, lysine, alanine, or phenylalanine;
each R^{j} is independently fluorine, chlorine, methyl, or —C(O)NHCH₃; and
subscript k is an integer ranging from 0 to 4.

28. The Antibody Drug Conjugate compound of claim 27, or a pharmaceutically acceptable salt thereof, represented by the structure of:

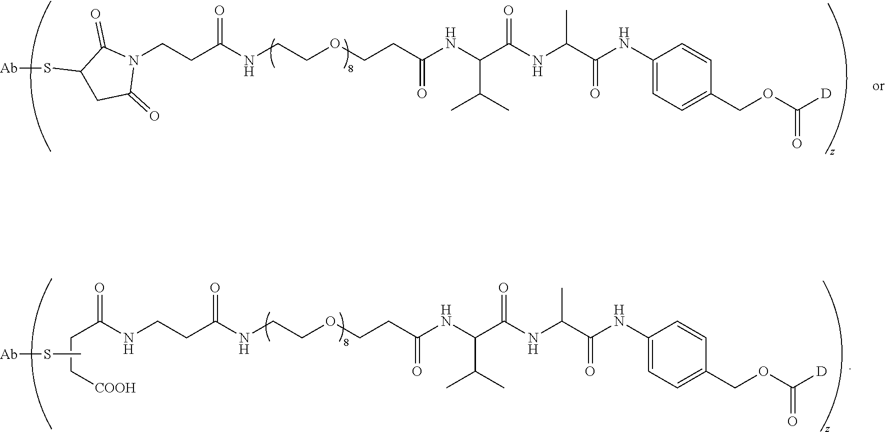

29. The Antibody Drug Conjugate compound of claim 21 or 22, or a pharmaceutically acceptable salt thereof, represented by the structure of: 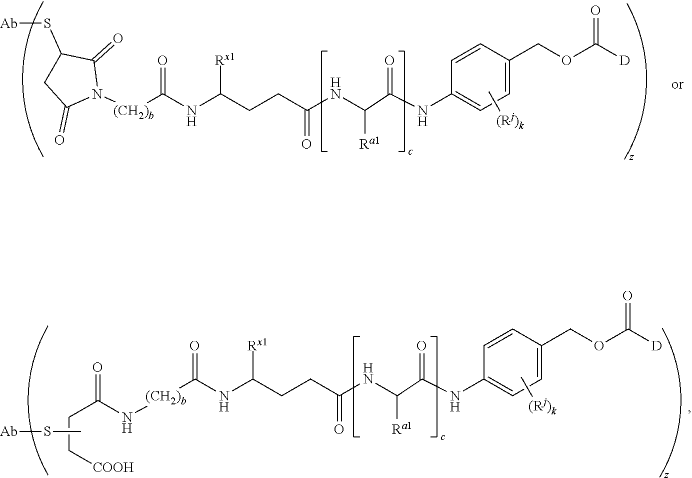
wherein
S is a sulfur atom of the Ab;
subscript b is an integer ranging from 1 to 5;
subscript c is an integer ranging from 1 to 3;
R^{x1} is hydrogen, —COOH, —C(O)NHCH₃, or —(C(O)NHCH₂)ₓ(CH₂OCH₂)_{y}R^{x2}wherein R^{x2} is —CH₂NHC(O)CH₃, —CH₂NH₂, or —CH₂C(O)OR^{x2a}, wherein R^{x2a} is hydrogen or C₁-C₆ alkyl;
each R^{a1} is independently the side chain of valine, glutamic acid, tryptophan, glycine, citrulline, lysine, alanine, or phenylalanine;
each R^{j} is independently fluorine, chlorine, methyl, or —C(O)NHCH₃; and
subscript k is an integer ranging from 0 to 4.

30. The Antibody Drug Conjugate compound of claim 29, or a pharmaceutically acceptable salt thereof, represented by the structure of:

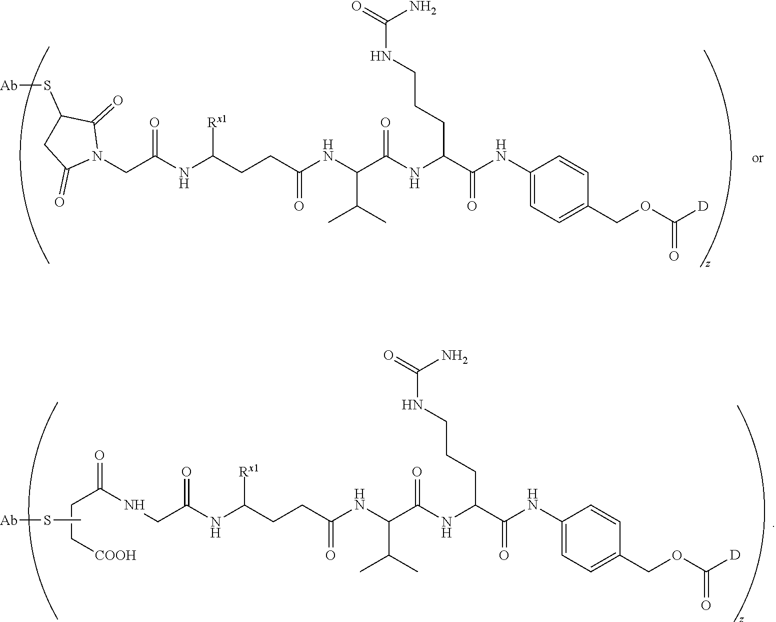

31. The Antibody Drug Conjugate compound of claim 29 or 30, or a pharmaceutically acceptable salt thereof, wherein RX is

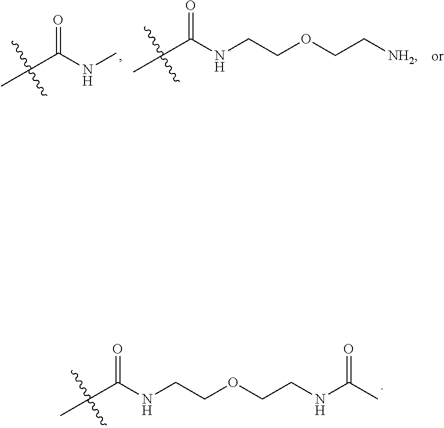

32. The Antibody Drug Conjugate compound of claim 21 or 22, or a pharmaceutically acceptable salt thereof, represented by the structure of: 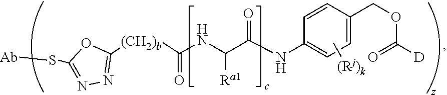
wherein
S is a sulfur atom of the Ab;
subscript b is an integer ranging from 1 to 5;
subscript c is 1 or 2;
each R^{a1} is independently the side chain of valine or citrulline;
each R^{j} is independently fluorine, chlorine, methyl, or —C(O)NHCH₃; and
subscript k is an integer ranging from 0 to 4.

33. The Antibody Drug Conjugate compound of claim 32, or a pharmaceutically acceptable salt thereof, represented by the structure of:

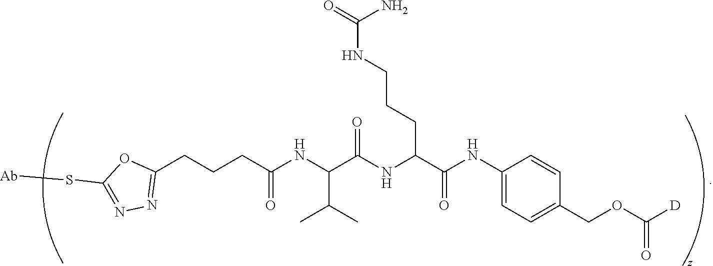

34. The Antibody Drug Conjugate compound of claim 33, or a pharmaceutically acceptable salt thereof, represented by the structure of:

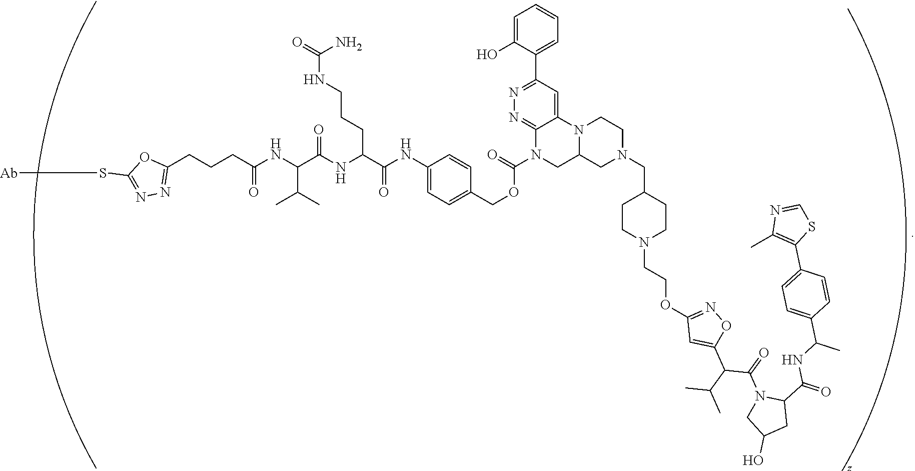

35. The Antibody Drug Conjugate compound of claim 21 or 22, or a pharmaceutically acceptable salt thereof, represented by the structure of: 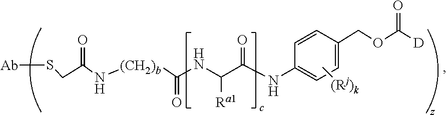
wherein
S is a sulfur atom of the Ab;
subscript b is an integer ranging from 1 to 5;
subscript c is 1 or 2;
each R^{a1} is independently the side chain of valine or citrulline;
each R^{j} is independently fluorine, chlorine, methyl, or —C(O)NHCH₃; and
subscript k is an integer ranging from 0 to 4.

36. The Antibody Drug Conjugate compound of claim 35, or a pharmaceutically acceptable salt thereof, represented by the structure of:

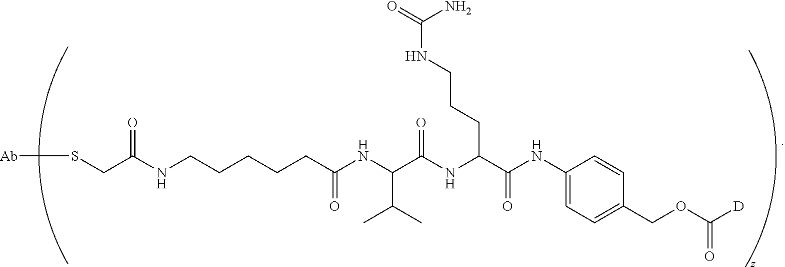

37. The Antibody Drug Conjugate compound of claim 36, or a pharmaceutically acceptable salt thereof, represented by the structure of:

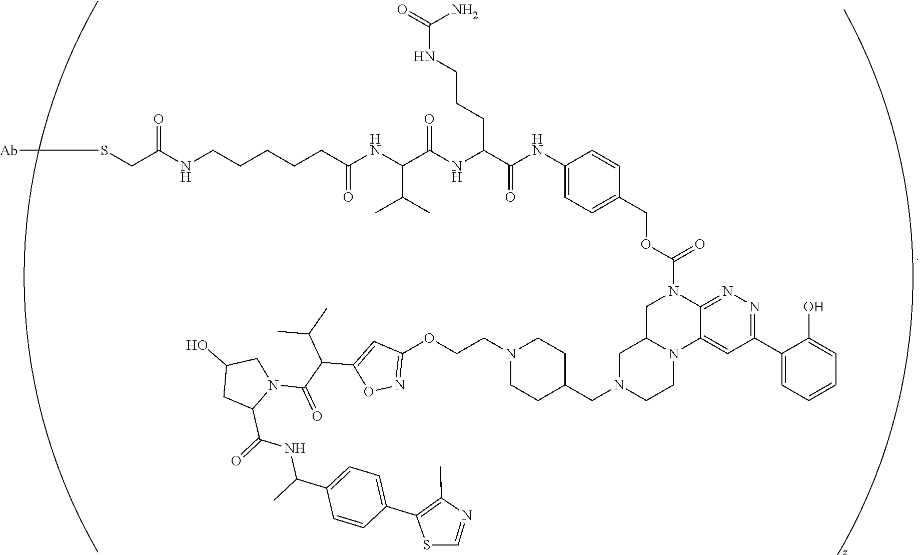

38. The Antibody Drug Conjugate compound of claim 21 or 22, or a pharmaceutically acceptable salt thereof, represented by the structure of: 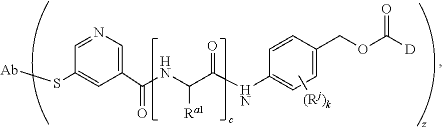
wherein
S is a sulfur atom of the Ab;
subscript c is an integer ranging from 1 to 4;
each R^{a1} is independently the side chain of valine or citrulline;
each R^{j} is independently fluorine, chlorine, methyl, or —C(O)NHCH₃; and
subscript k is an integer ranging from 0 to 4.

39. The Antibody Drug Conjugate compound of claim 38, or a pharmaceutically acceptable salt thereof, represented by the structure of:

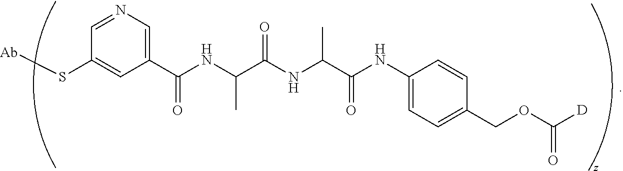

40. The Antibody Drug Conjugate compound of claim 21 or 22, or a pharmaceutically acceptable salt thereof, represented by the structure of: 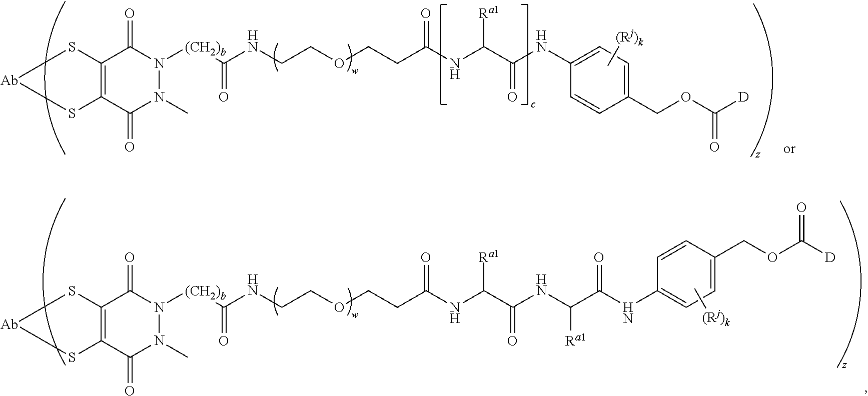
wherein
S is a sulfur atom of the Ab;
subscript c is an integer ranging from 1 to 4;
each R^{a1} is independently the side chain of valine or citrulline;
each R^{j} is independently fluorine, chlorine, methyl, or —C(O)NHCH₃; and
subscript k is an integer ranging from 0 to 4.

41. The Antibody Drug Conjugate compound of claim 40, or a pharmaceutically acceptable salt thereof, represented by the structure of:

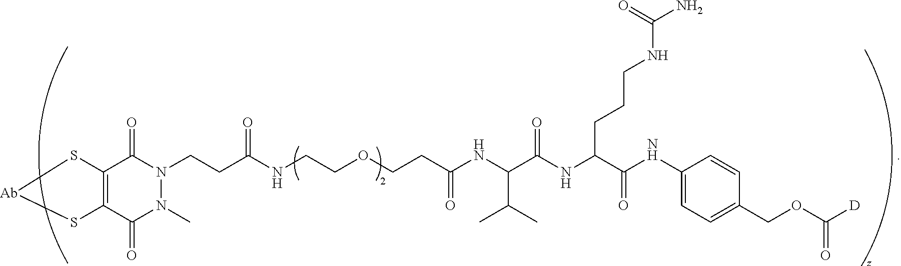

42. The Antibody Drug Conjugate compound of claim 21 or 22, or a pharmaceutically acceptable salt thereof, represented by the structure of:

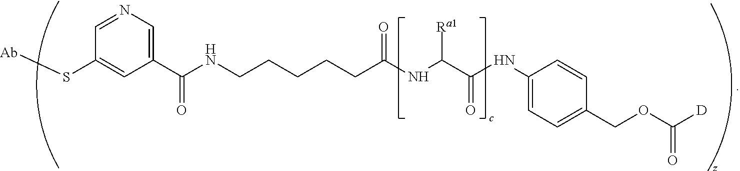

43. The Antibody Drug Conjugate compound of claim 41, or a pharmaceutically acceptable salt thereof, represented by the structure of:

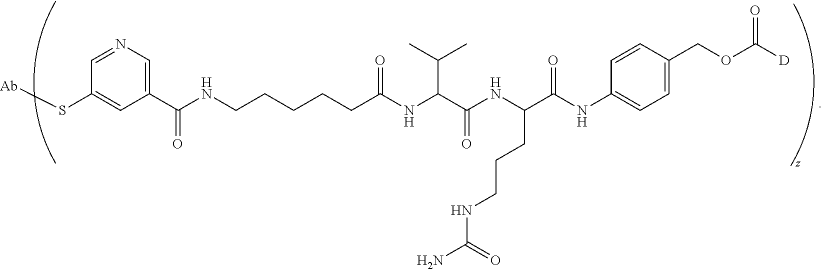

44. The Antibody Drug Conjugate compound of claim 21 or 22, or a pharmaceutically acceptable salt thereof, represented by the structure of: 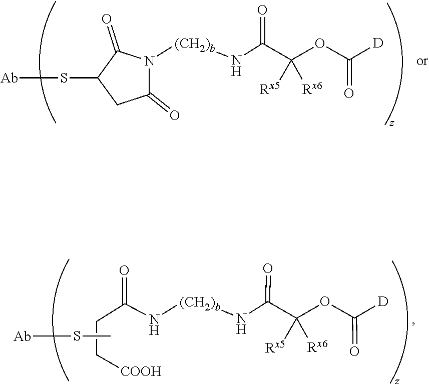
wherein
subscript b is an integer ranging from 1 to 5;
R^{x5} is hydrogen or C₁-C₆ alkyl; and
R^{x6} is hydrogen, C₁-C₆ alkyl, or 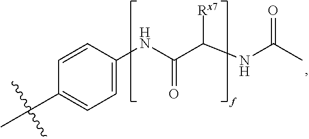
wherein subscript f is an integer ranging from 0 to 4 and each R^{x7} is independently hydrogen, C₁-C₆ alkyl, or an independently selected side chain of an amino acid.

45. The Antibody Drug Conjugate compound of claim 44, or a pharmaceutically acceptable salt thereof, represented by the structure of: 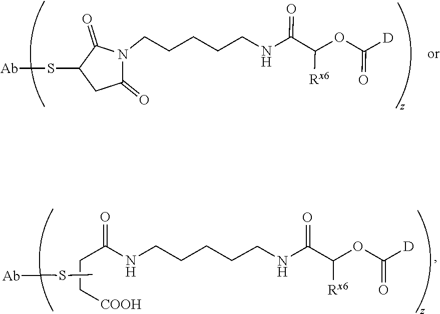
wherein
R^{x6} is 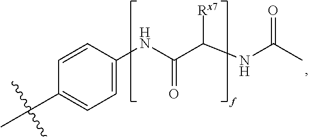
wherein subscript f is an integer ranging from 0 to 4 and each R^{x7} is independently hydrogen, C₁-C₆ alkyl, or an independently selected side chain of an amino acid.

46. The Antibody Drug Conjugate compound of claim 45, or a pharmaceutically acceptable salt thereof, wherein R^{x6} is

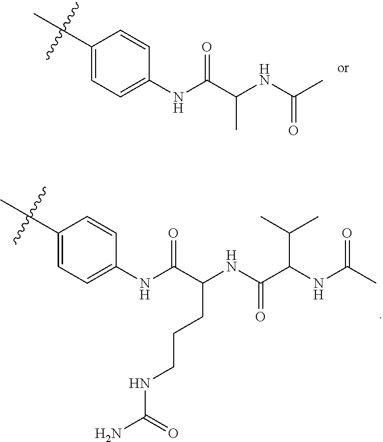 [SMILES: *c1ccc(NC(=O)C(C)NC(C)=O)cc1 ; *c1ccc(NC(=O)C(CCCNC(N)=O)NC(=O)C(NC(C)=O)C(C)C)cc1]

47. The Antibody Drug Conjugate compound of claim 21 or 22, or a pharmaceutically acceptable salt thereof, represented by the structure of: 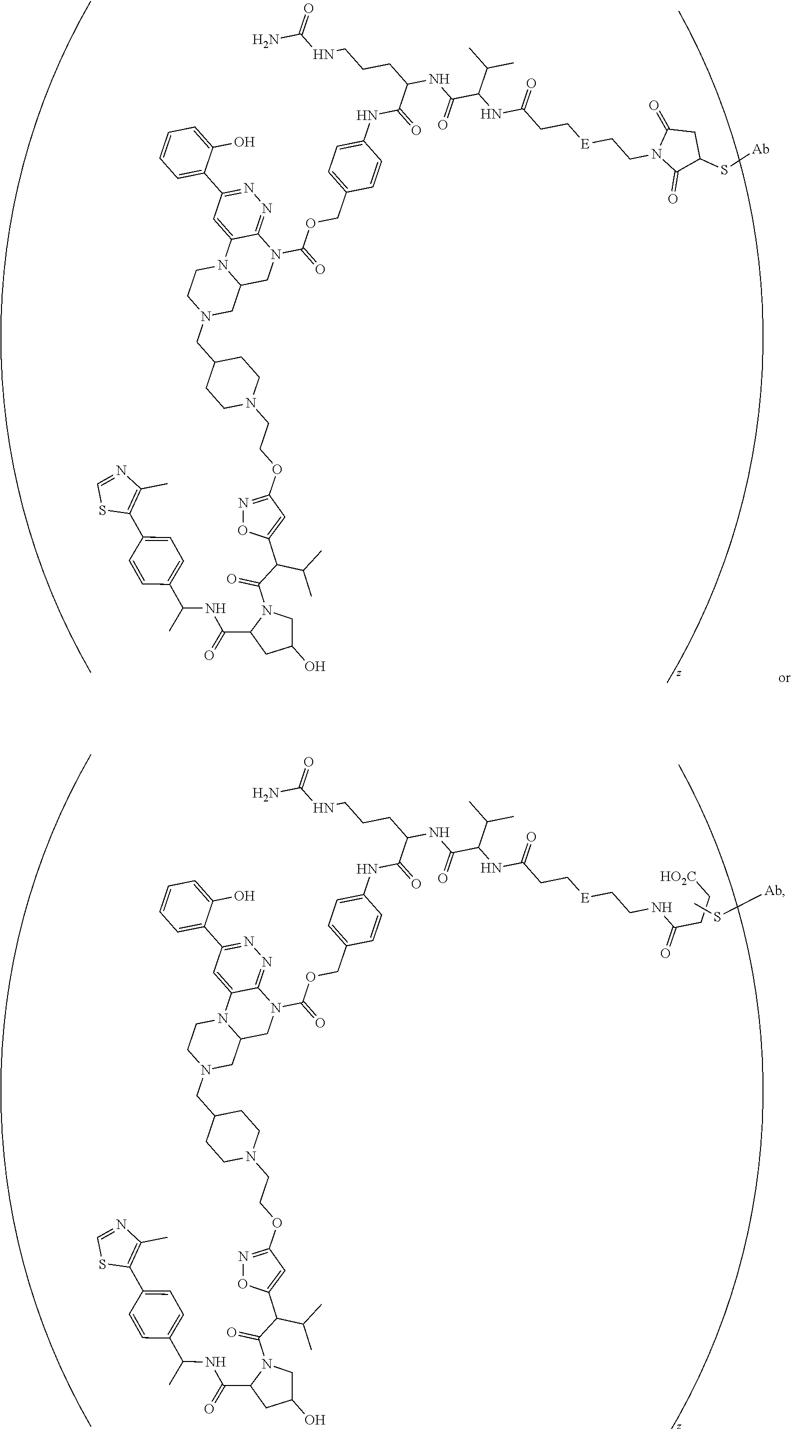
wherein E is —CH₂— or —O—.

48. The Antibody Drug Conjugate compound of claim 21 or 22, or a pharmaceutically acceptable salt thereof, represented by the structure of: 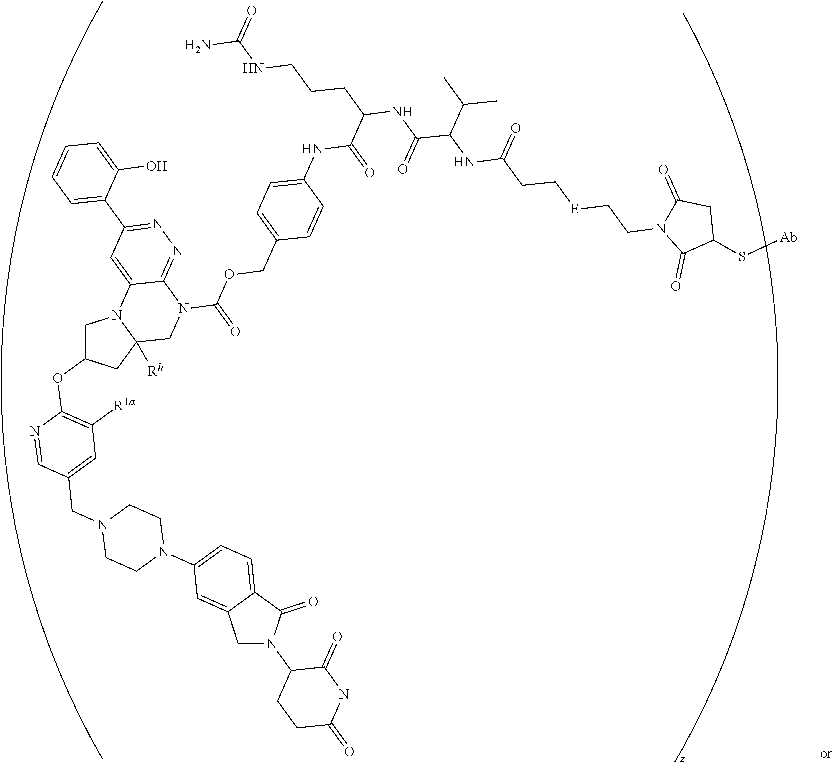 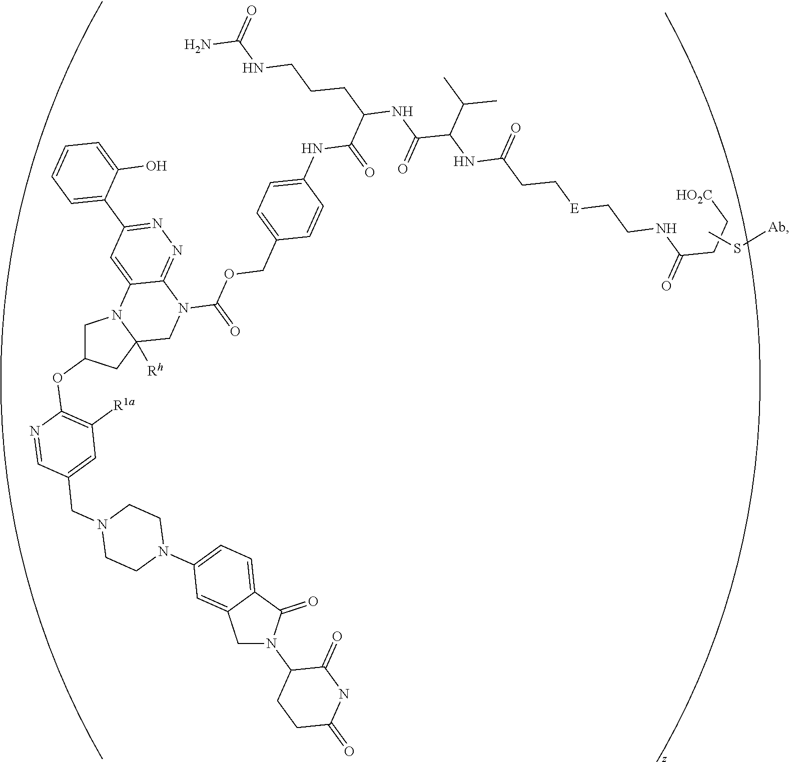
wherein
E is —CH₂— or —O—;
R^{h} is methyl or ethyl; and
R^{1a} is hydrogen, methyl, or fluorine.

49. The Antibody Drug Conjugate compound of claim 21 or 22, or a pharmaceutically acceptable salt thereof, represented by the structure of: 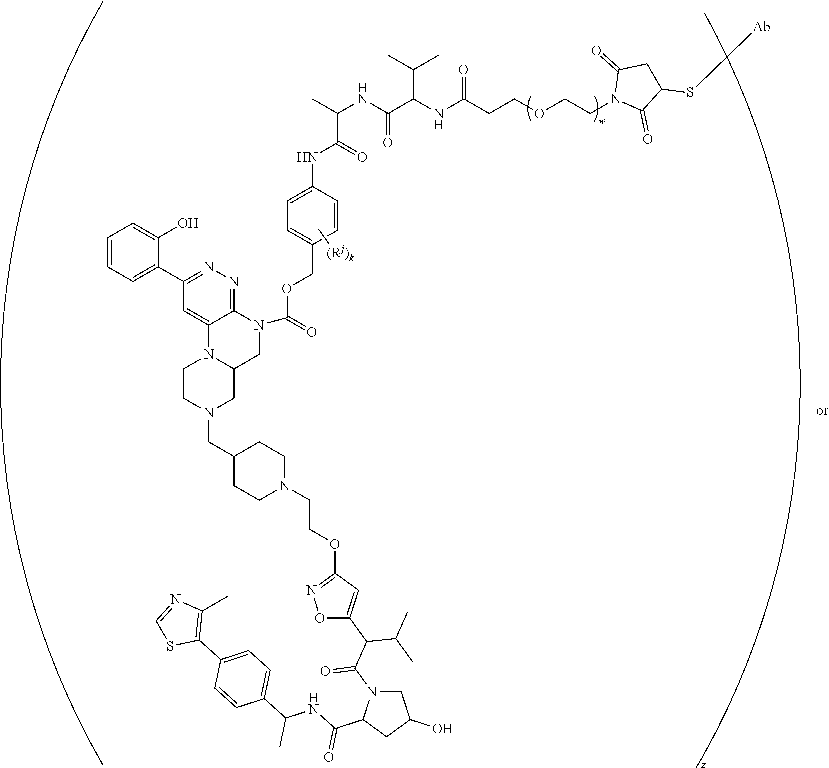 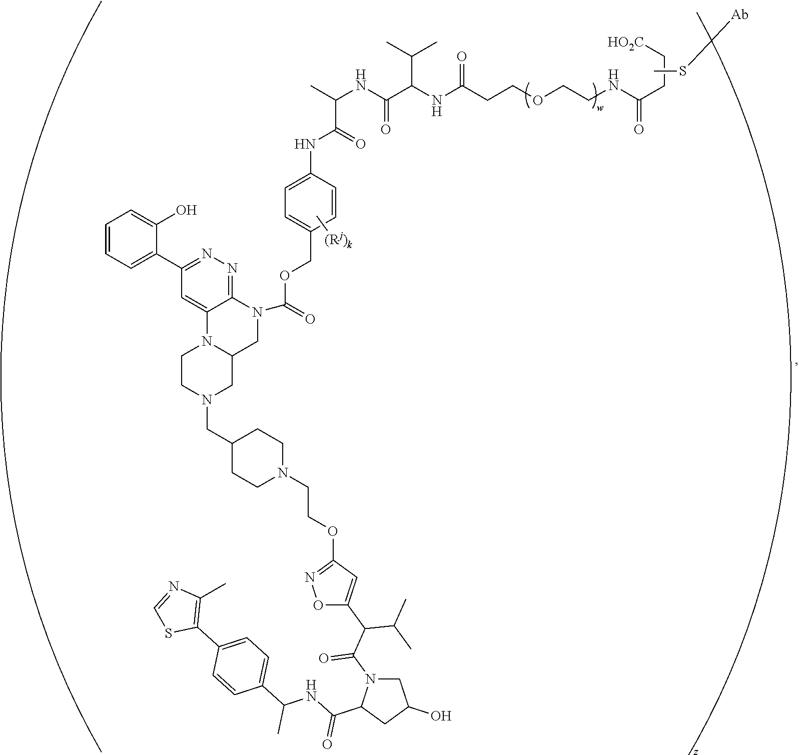
wherein subscript w is an integer ranging from 1 to 8; R^{j} is methyl, —F, —Cl, or —C(O)NHCH₃; and subscript k is 0 or 1.

50. The Antibody Drug Conjugate compound of claim 21 or 22, or a pharmaceutically acceptable salt thereof, represented by the structure of:

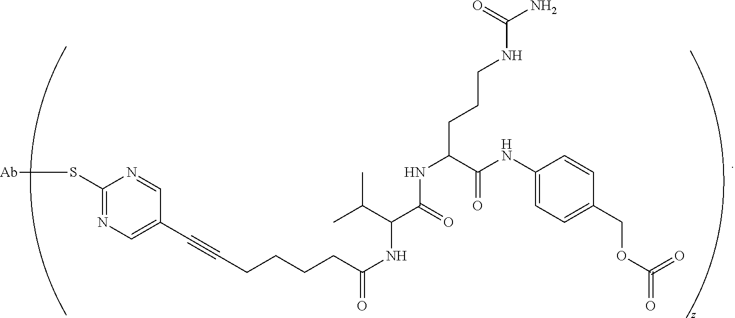

51. The Antibody Drug Conjugate compound of claim 21 or 22, or a pharmaceutically acceptable salt thereof, represented by the structure of: 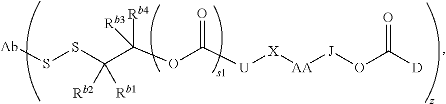
wherein
R^{b1} and R^{b2} are each independently hydrogen, C₁-C₆ alkyl, or both R^{b1} and R^{b2}, together with the carbon to which they are attached, comprise a C₄-C₆ cycloalkyl or a C₄-C₆ heterocycloalkyl;
R^{b3} and R^{b4} are each independently hydrogen or C₁-C₆ alkyl;
subscript s1 is 0 or 1; and
U, X, and AA are each absent.

52. The Antibody Drug Conjugate compound of claim 51, or a pharmaceutically acceptable salt thereof, represented by the structure of: 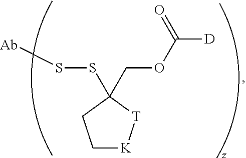
wherein T is absent or is —CH₂CH₂—; K is —CH₂— or —NH₂—.

53. The Antibody Drug Conjugate compound of claim 51 or 52, or a pharmaceutically acceptable salt thereof, represented by the structure of:

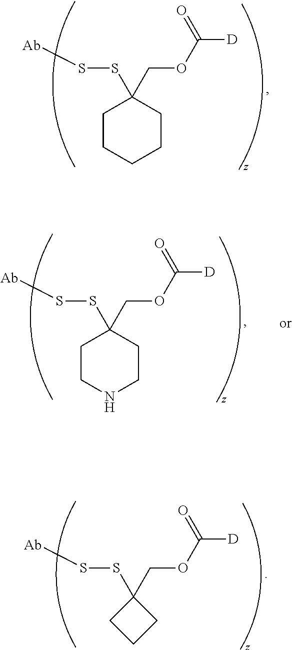

54. The Antibody Drug Conjugate compound of claim 51, or a pharmaceutically acceptable salt thereof, represented by the structure of:

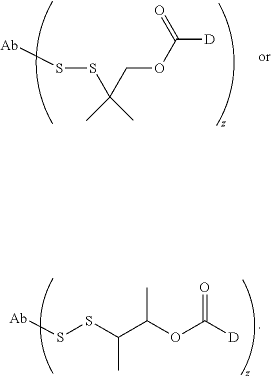

55. The Antibody Drug Conjugate compound of claim 6 or 7, or a pharmaceutically acceptable salt thereof, represented by the structure of: 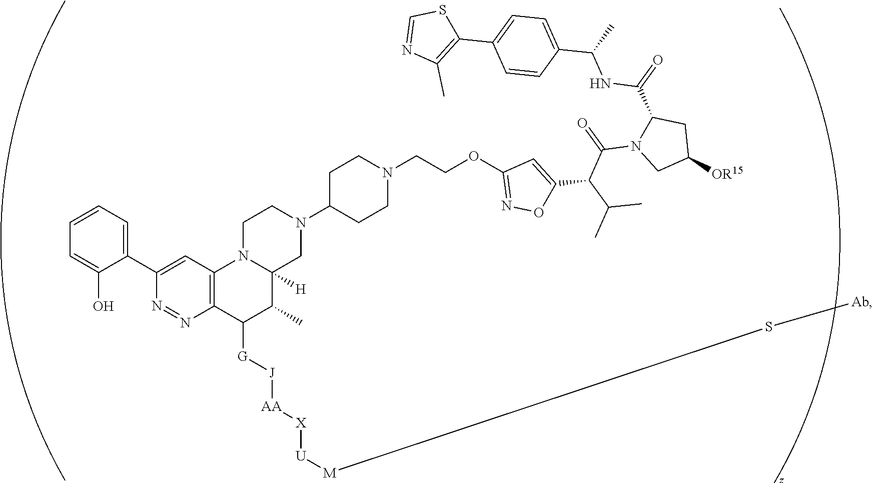
wherein S is a sulfur atom of the Ab and R¹⁵ is hydrogen or —PO₃H₂.

56. The Antibody Drug Conjugate compound of claim 55, or a pharmaceutically acceptable salt thereof, represented by the structure of: 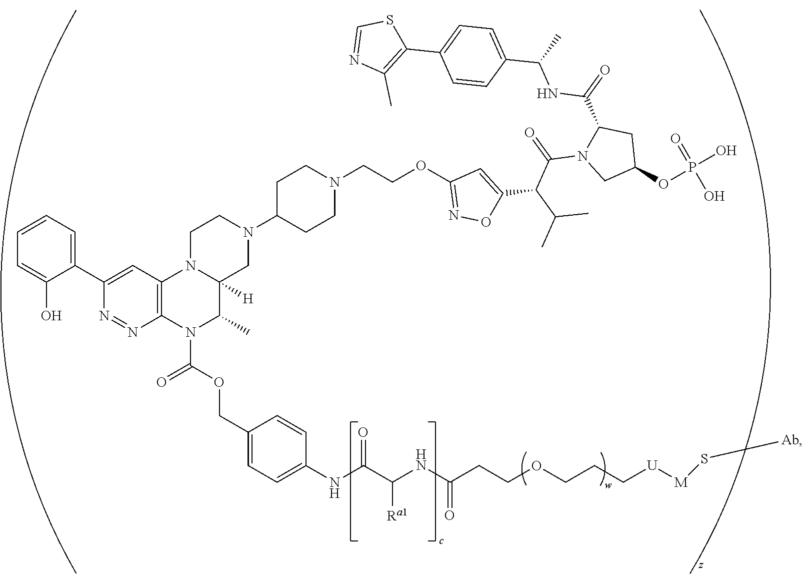
wherein
S is a sulfur of the Ab;
subscript c is an integer ranging between 1 and 4;
subscript w is an integer ranging between 1 and 8; and
U is absent or is —CH₂—(C═O)—(NH)—.

57. The Antibody Drug Conjugate compound of claim 6 or 7, or a pharmaceutically acceptable salt thereof, represented by the structure of: 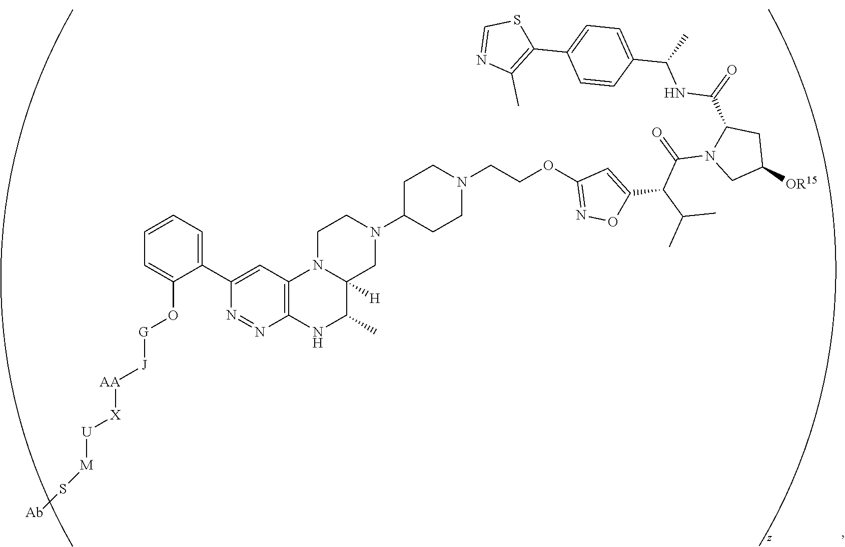
wherein S is a sulfur of the Ab and R¹⁵ is hydrogen or —PO₃H₂.

58. The Antibody Drug Conjugate compound of claim 57, or a pharmaceutically acceptable salt thereof, represented by the structure of: 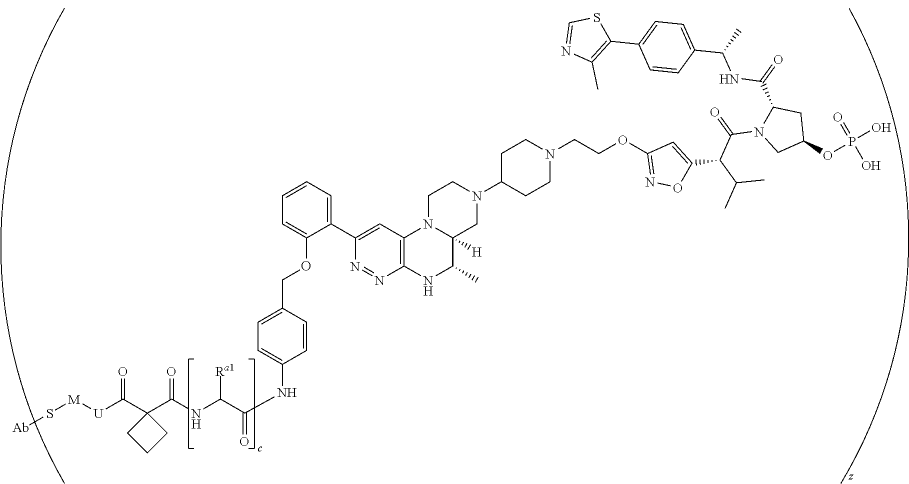
wherein
S is a sulfur of the Ab;
subscript c is an integer ranging between 1 and 4; and
U is —(CH₂CH₂O)_{w}CH₂CH₂(NH)—, wherein subscript w is an integer ranging between 1 and 4.

59. An Antibody Drug Conjugate compound, or a pharmaceutically acceptable salt thereof, having the structure of: 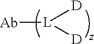
wherein,
Ab is an antibody or an antigen-binding fragment thereof,
L is a linker;
D is a degrader compound of Formula (I):
PTM-ULM (I)
wherein,
PTM is a moiety of Formula IA: 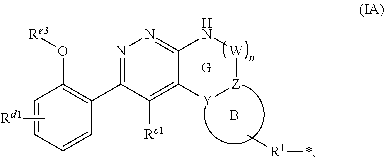
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase; and
subscript z is an integer ranging from 1 to 14.

60. The Antibody Drug Conjugate compound of claim 59, or a pharmaceutically acceptable salt thereof, wherein L is a linker of Formula (III): 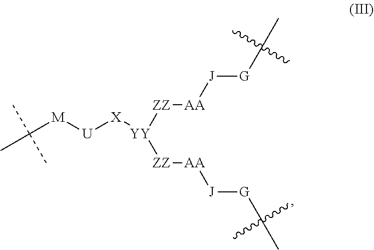
wherein,
M is selected from the group consisting of: 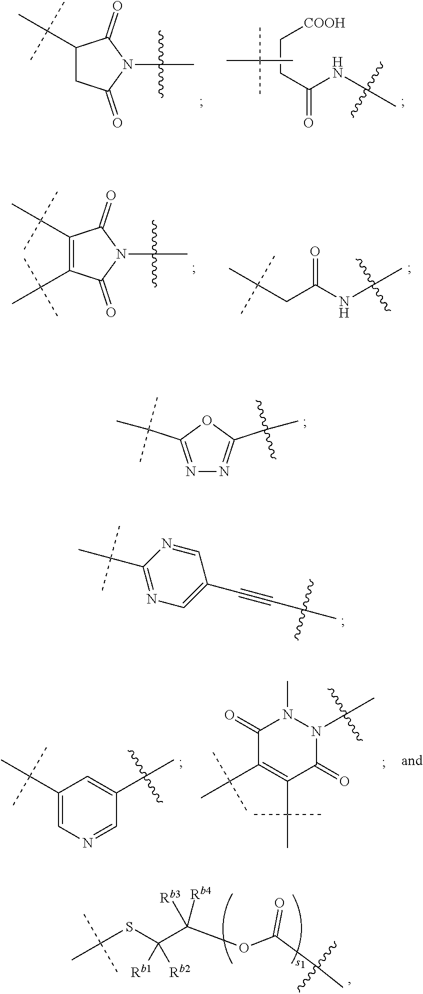
wherein
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1,
wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—OC(O)—, wherein R^{x5} is hydrogen or C₁-C₆ alkyl and R^{x6} is hydrogen, C₁-C₆ alkyl, or 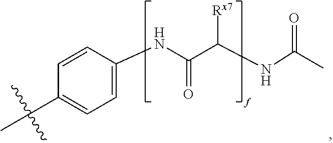
wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of: 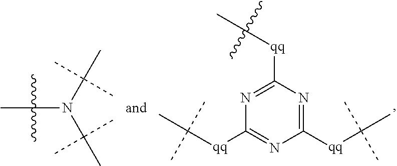
wherein
each qq is independently —N(R^{y1})— or —O—, wherein R^{y1} is hydrogen or C₁-C₆ alkyl;
the wavy line indicates the point of attachment to X, when present, or U, when X is absent, or M, when X and U are absent; and
each dashed line indicates the point of attachment to ZZ when ZZ is present, or to AA when ZZ is absent, or to J when AA and ZZ are absent, or to G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of: 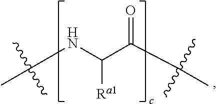
wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of: 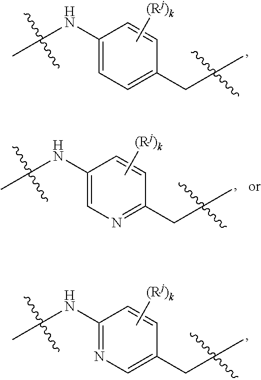
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
each G is independently absent, 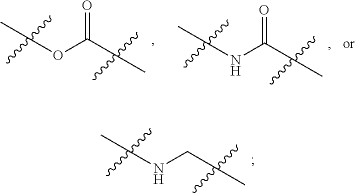
and
wherein the wavy line to M of Formula (III) indicates the point of covalent attachment to Ab and the wavy line to each G of Formula (III) indicates the point of covalent attachment to D,
wherein subscript z is an integer ranging from 1 to 14.

61. The Antibody Drug Conjugate compound of claim 59 or 60, or a pharmaceutically acceptable salt thereof, wherein L has the structure of:

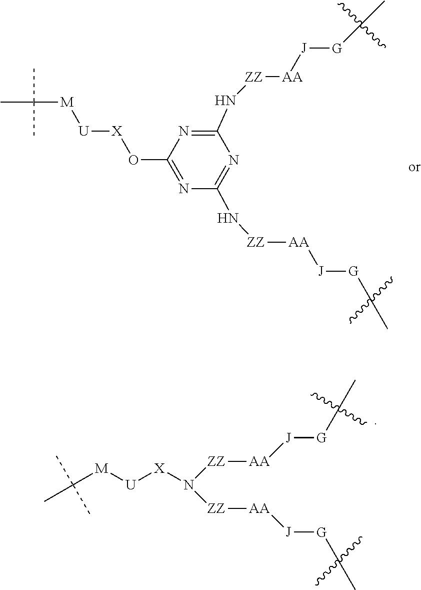

62. The Antibody Drug Conjugate compound of any one of claims 59 to 61, or a pharmaceutically acceptable salt thereof, wherein L has the structure of: 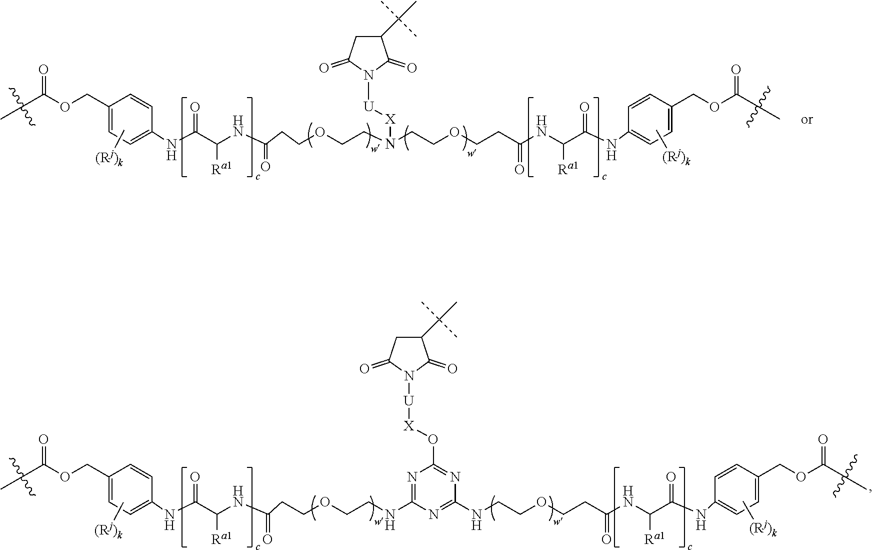
wherein
U is absent or is —(CH₂)_{b}(R^{v1})ₛₛ(C═O)ᵤ(NH₁)ᵥ—, wherein subscript b is 0, 1, 2, 3, 4,PG-05C or 5; subscript ss is 0 or 1; subscript u is 0 or 1; subscript v is 0 or 1; R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl;
X is absent or is —(CH₂CH₂O)_{w}CH₂CH₂—(C═O)_{d}—, wherein subscript w is an integer ranging from 0 to 8 and subscript d is 0 or 1;
subscript c is an integer ranging from 1 to 12 and each R^{a1} is independently —COOH, —NH₂, or an independently selected side chain of an amino acid, wherein each amino acid is selected from the group consisting of alanine, valine, citrulline, glutamic acid, tryptophan, glycine, phenylalanine, and lysine;
each R^{j} is independently fluorine, chlorine, methyl, or —C(O)NHCH₃; and
subscript k is an integer ranging from 0 to 4.

63. An Antibody Drug Conjugate compound of claim 59, or a pharmaceutically acceptable salt thereof, wherein L is a linker of Formula (IIb): 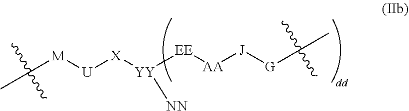
wherein,
subscript dd is 2;
M is selected from the group consisting of: 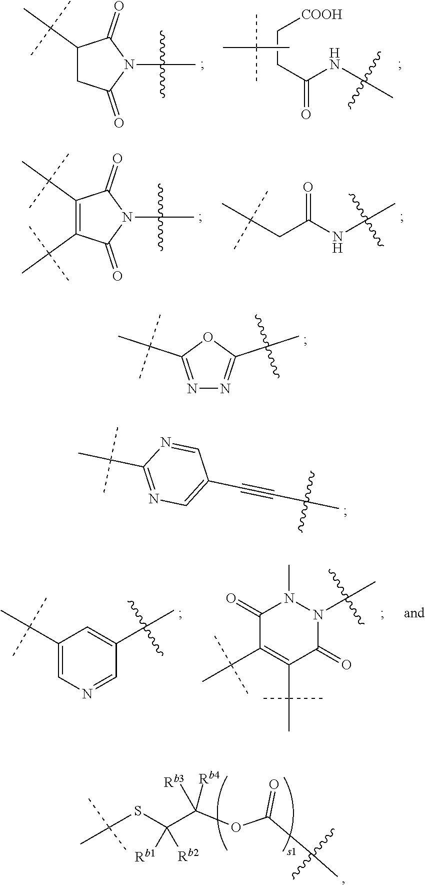
wherein
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1,
wherein the dashed line indicates the point of covalent attachment to the Ab and the wavy line indicates the point of covalent attachment to the remainder of the structure of L;
U is absent or is —(CH₂)_{b}(R^{v1})ₛₛ(C═O)ᵤ(NH)ᵥ— wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—, wherein R^{x5} is hydrogen or C₁-C₆ alkyl and R^{x6} is hydrogen, C₁-C₆ alkyl, or 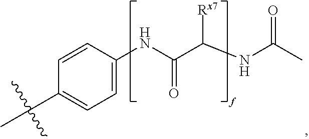
wherein subscript f is an integer ranging from 0 to 4 and each R^{x7} is independently hydrogen, —COOH, —NH₂, C₁-C₆ alkyl, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit having the structure of: 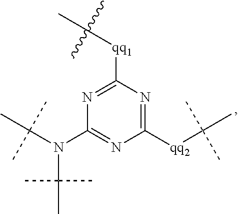
wherein
qq₁ and qq₂ are each independently —N(R^{y1})— or —O—, wherein R^{y1} is hydrogen or C₁-C₆ alkyl, and
the wavy line of YY indicates the point of attachment to X, when present, or to U when X is absent, or to M when X and U are absent; and
the dashed lines of YY indicate the point of attachment to EE when EE is present, or to AA when EE is absent, or to J when AA and EE are absent, or to G when AA, EE, and J are absent, and to NN;
NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
each EE is independently absent or —(CH₂CH₂O)_{x′}CH₂CH₂C(O)—, wherein subscript x′ is an integer ranging from 0 to 16;
each AA is independently absent or has the structure of: 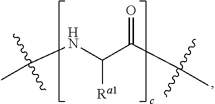
wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of: 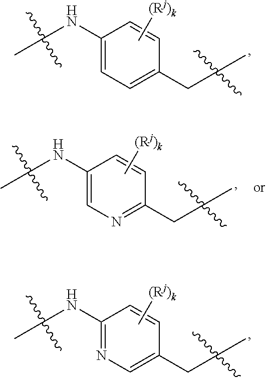
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
each G is independently absent, 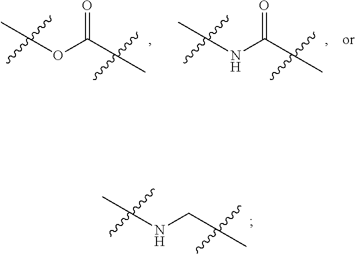
and
wherein the wavy line to M of Formula (IIb) indicates the point of covalent attachment to Ab and the wavy line to G of Formula (IIb) indicates the point of covalent attachment to an independently selected D.

64. The Antibody Drug Conjugate compound of claim 63, or a pharmaceutically acceptable salt thereof, wherein L has the structure of: 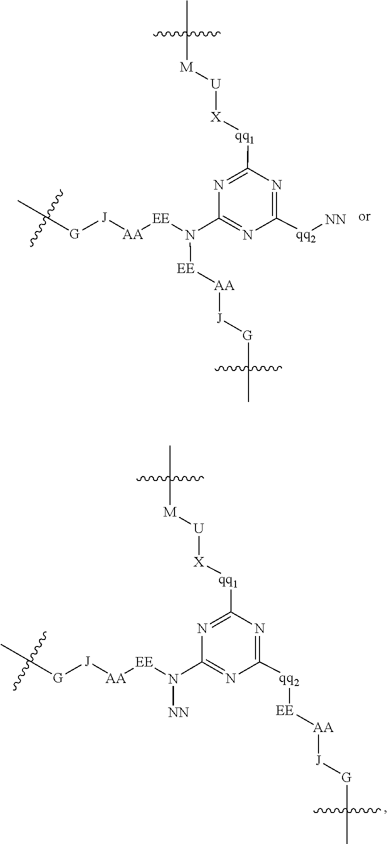
wherein qq₁ and qq₂ are each independently —N(R^{y1} )- or —O—, wherein R^{y1} is hydrogen or C₁-C₆ alkyl.

65. The Antibody Drug Conjugate compound of any one of claims 1 to 64, or a pharmaceutically acceptable salt thereof, wherein z is an integer ranging from 2 to 8.

66. The Antibody Drug Conjugate compound of any one of claims 1 to 65, or a pharmaceutically acceptable salt thereof, wherein z is 4.

67. The Antibody Drug Conjugate compound of any one of claims 1 to 66, or a pharmaceutically acceptable salt thereof, wherein z is 6.

68. The Antibody Drug Conjugate compound of any one of claims 1 to 67, or a pharmaceutically acceptable salt thereof, wherein Ab binds to prostate-specific membrane antigen (PSMA).

69. The Antibody Drug Conjugate compound of any one of claims 1 to 68, or a pharmaceutically acceptable salt thereof, wherein Ab is an anti-PSMA antibody.

70. The Antibody Drug Conjugate compound of any one of claims 1 to 67, or a pharmaceutically acceptable salt thereof, wherein Ab binds to CD33 antigen.

71. The Antibody Drug Conjugate compound of claim 70, or a pharmaceutically acceptable salt thereof, wherein Ab is an anti-CD33 antibody.

72. A pharmaceutical composition comprising an Antibody Drug Conjugate compound of any one of claims 1 to 71, or claims 135 or 136, and at least one pharmaceutically acceptable excipient.

73. A method of treating cancer in a subject in need thereof comprising administering to the subject an effective amount of an Antibody Drug Conjugate compound of any one of claims 1 to 71, or claims 135 or 136, or a pharmaceutically acceptable salt thereof, or a pharmaceutical composition of claim 72.

74. The method of claim 73, wherein the cancer is prostate cancer or acute myeloid leukemia (AML).

75. A Degrader-Linker compound, or a pharmaceutically acceptable salt or solvate thereof, having the structure of:
L′-D
wherein,
L′ is a linker precursor; and
D is a degrader compound of Formula (I):
PTM-ULM (I)
wherein,
PTM is a moiety of Formula IA: 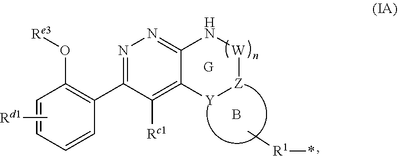
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z; and
ULM is a small molecule E3 Ubiquitin Ligase binding moiety that binds a Von Hippel-Lindau E3 Ubiquitin Ligase or a Cereblon E3 Ubiquitin Ligase.

76. A Degrader-Linker compound, or a pharmaceutically acceptable salt or solvate thereof, having the structure of:
L′-D
wherein,
D is a degrader compound of Formula (i):
PTM-ULM (i),
wherein,
PTM is a moiety of Formula ia: 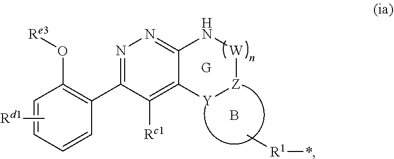
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, or C₁₋₃ alkyl, absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase;
L′ is a linker precursor of Formula (ii): 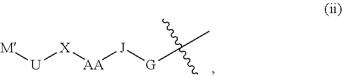
wherein
M′ is selected from the group consisting of: 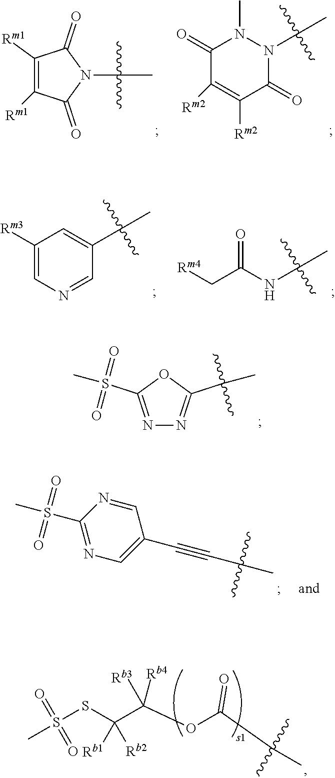
wherein
each R^{m1} and R^{m2} is independently hydrogen, halogen, or —S-Ph;
R^{m3} and R^{m4} are each halogen;
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1;
wherein the wavy line indicates the point of covalent attachment to the remainder of the structure of L′;
U is absent or is —(CH₂)_{b}(R^{v1})ₛₛ(C═O)ᵤ(NH)ᵥ— wherein subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—, wherein R^{x5} is hydrogen or C₁-C₆ alkyl and R^{x6} is hydrogen, C₁-C₆ alkyl, or 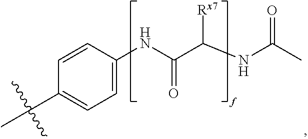
wherein subscript f is an integer ranging from 0 to 4 and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
AA is absent or has the structure of: 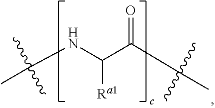
wherein subscript c is an integer ranging from 1 to 12; each R^{a1} is independently —COOH, —NH₂, or an independently selected side chain of an amino acid;
J is absent, —(R^{j3})N(C(R^{j1})(R^{j2}))ₘ₁—, or has the structure of: 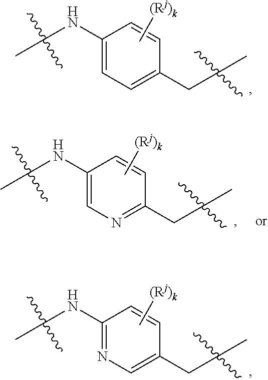
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
G is absent, 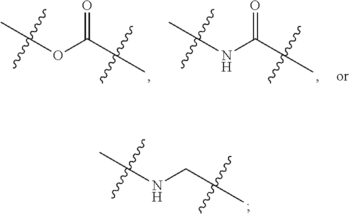
and
wherein the wavy line to G in Formula (ii) indicates the point of covalent attachment to D.

77. A Degrader-Linker compound, or a pharmaceutically acceptable salt or solvate thereof, having the structure of:
L′-D
wherein,
D is a degrader compound of Formula (i):
PTM-ULM (i),
wherein,
PTM is a moiety of Formula ia: 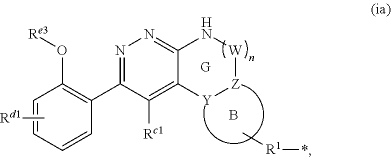
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, —CH₂—O—P(O)(OR^{g})₂, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
Z and Y are each independently N; CR^{h} wherein R^{h}=H, or C₁₋₃ alkyl, absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z;
ULM is a small molecule E3 Ubiquitin Ligase binding moiety that binds a Von Hippel-Lindau E3 Ubiquitin Ligase or a Cereblon E3 Ubiquitin Ligase;
L′ is a linker precursor of Formula (iia): 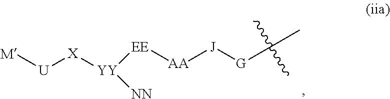
wherein
M′ is selected from the group consisting of 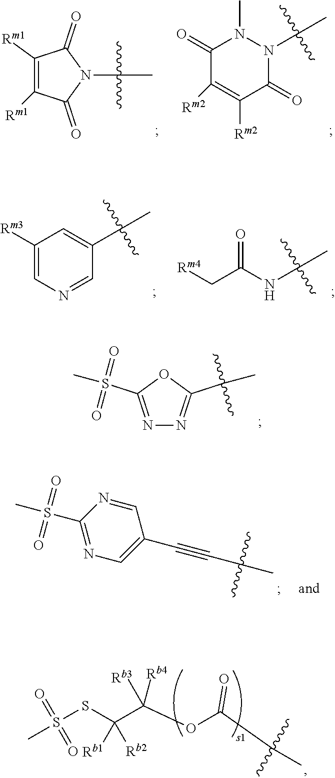
wherein
each R^{m1} and R^{m2} is independently hydrogen, halogen, or —S-Ph;
R^{m3} and R^{m4} are each halogen;
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1;
wherein the wavy line indicates the point of covalent attachment to the remainder of the structure of L′;
U is absent or is —(CH₂)_{b}(R^{v1})ₛₛ(C═O)ᵤ(NH)ᵥ— wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—, wherein R^{x5} is hydrogen or C₁-C₆ alkyl and R^{x6} is hydrogen, C₁-C₆ alkyl, or 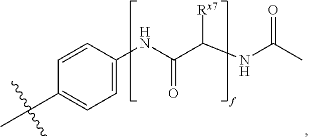
wherein subscript f is an integer ranging from 0 to 4 and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of: 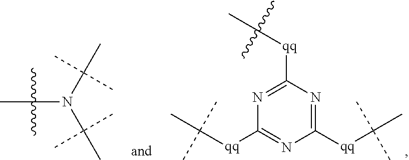
wherein
each qq is independently —N(R^{y1})— or —O—, wherein R^{y1} is hydrogen or C₁-C₆ alkyl,
one dashed line indicates the point of covalent attachment to EE when EE is present, or to AA when EE is absent, or to J when AA and EE are absent, or to G when AA, EE, and J are absent, and the other dashed line indicates the point of covalent attachment to NN, wherein the wavy line indicates the point of covalent attachment to X, when X is present, or to U, when X is absent, or to M, when X and U are absent;
NN is —(CH₂CH₂O)_{k′}CH₂CH₂C(O)—Rⁿⁿ¹-, wherein subscript k′ is an integer ranging from 0 to 16 and Rⁿⁿ¹ is hydrogen, C₁-C₆ alkyl, or —OH;
EE is absent or —(CH₂CH₂O)_{x′}CH₂CH₂C(O)—, wherein subscript x′ is an integer ranging from 0 to 16;
AA is absent or has the structure of: 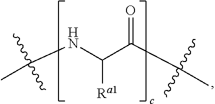
wherein subscript c is an integer ranging from 1 to 12; each R^{a1} is independently —COOH, —NH₂, or an independently selected side chain of an amino acid;
J is independently absent, —(R^{j3})N(C(R^{j1})(R^{j2}))ₘ₁—, or has the structure of: 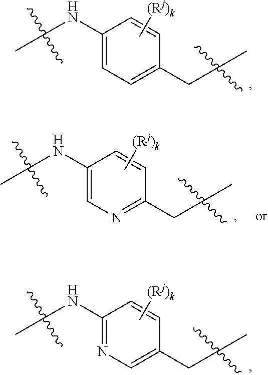
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
G is absent, 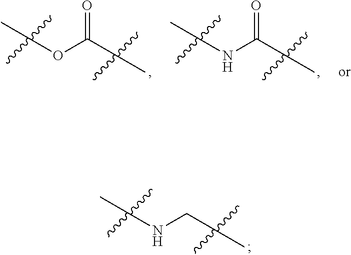
and
wherein the wavy line to G in Formula (iia) indicates the point of covalent attachment to D.

78. The Degrader-Linker compound of claim 76 or 77, or a pharmaceutically acceptable salt thereof, wherein R^{j} is a covalent bond.

79. The Degrader-Linker compound of claim 76 or 77, or a pharmaceutically acceptable salt thereof, wherein R^{j} is a chemical moiety represented by the formula:
-(A)_{q}-,
wherein:
q is an integer from 1 to 14;
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a} (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₄, NR^{1c}C(═NCN)NR^{1d}NR^{1c}C(═NCN), NR^{1c}C(═CNO₂)NR^{1d} 3-11 membered cycloalkyl, optionally substituted with 0-6 R^{1a} and/or R^{1b} groups, 3-11 membered heteocyclyl optionally substituted with 0-6 R^{1a} and/or R^{1b} groups, aryl optionally substituted with 0-6 R^{1a} and/or R^{1b} groups, and heteroaryl optionally substituted with 0-6 R^{1a} and/or R^{1b} groups,
wherein R^{1a}, R^{1b}, R^{1c}, R^{1d} and R^{1e} are each independently, —H, deuterium, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC1-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups.

80. The Degrader-Linker compound of any one of claims 76 to 79, or a pharmaceutically acceptable salt thereof, wherein ULM binds Von Hippel-Lindau E3 Ubiquitin Ligase.

81. The Degrader-Linker compound of claim 80, or a pharmaceutically acceptable salt thereof, wherein ULM is a moiety having the Formula ULM-I-VHL-1: 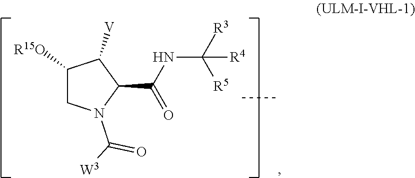
wherein
the dashed line ( [symbol] ) indicates the position of attachment of ULM-I-VHL-1 to R¹;
R¹⁵ is hydrogen or —PO₃H₂;
V is H or F;
R³ is optionally substituted phenyl, optionally substituted napthyl, or an optionally substituted 5-10 membered heteroaryl;
one of R⁴ or R⁵ is H, deuterium, haloalkyl, optionally substituted alkyl, optionally substituted cycloalkyl, optionally substituted heterocycloalkyl, —COR^{dv}, or CONR^{e1}R^{e2};
the other of R⁴ or R⁵ is H or deuterium;
or R⁴ and R⁵, together with the carbon atom to which they are both attached, form an optionally substituted 3-5 membered cycloalkyl or heterocyclyl;
W³ is an optionally substituted aryl, optionally substituted heteroaryl, or 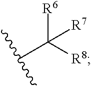
R⁶ and R⁷ are independently H, deuterium, optionally substituted alkyl, optionally substituted cycloalkyl, or optionally substituted haloalkyl,
or R⁶, R⁷, and the carbon atom to which they are attached form an optionally substituted cycloalkyl or optionally substituted heterocyclyl;
R⁸ is an optionally substituted heterocyclyl, optionally substituted heteroaryl, optionally substituted aryl, CONR^{av}R^{bv}, NR^{av}R^{bv}, 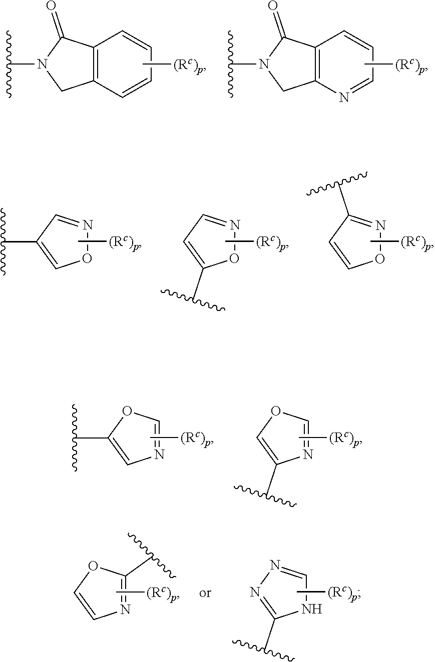
R^{av} is H or optionally substituted alkyl;
R^{bv} is H, —C(O)—* wherein * is a point of attachment to R¹, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{c} is independently H, halo, optionally substituted alkoxy, cyano, optionally substituted alkyl, haloalkyl, or haloalkoxy;
each R^{dv} is independently H, optionally substituted alkyl or NR^{e1}R^{e2};
each R^{e1} and R^{e2} is independently H, deuterium, or optionally substituted alkyl,
or R^{e1} and R^{e2} together with the nitrogen atom to which they are attached form a 4-7 membered heterocyclyl; and
p is 0, 1, 2, 3, or 4.

82. The Degrader-Linker compound of claim 80 or 81, wherein ULM-I-VHL-1 is a compound of formula: 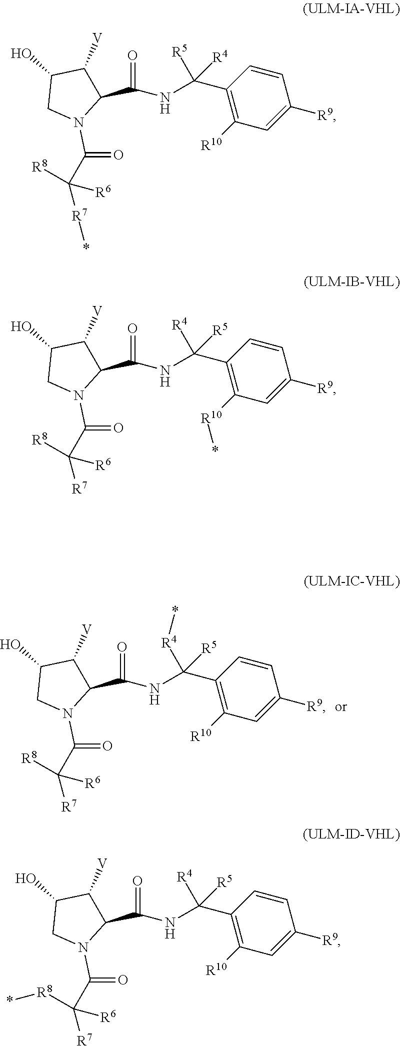
wherein * is a point of attachment of the ULM to R¹.

83. The Degrader-Linker compound of any one of claims 80 to 82, or a pharmaceutically acceptable salt thereof, wherein D has a structure of: 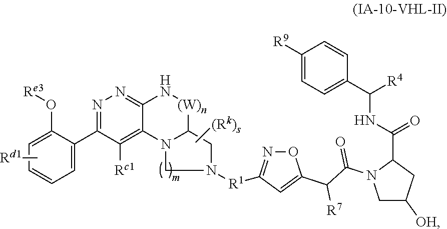
wherein
W is optionally substituted —CH₂—, —C(O)—, —S(O)—, or —S(O)₂—; wherein when n=2 or 3, only one W may be —C(O)—, —S(O)—, or —S(O)₂—;
n=0-3;
m=1-3;
R^{k}=H, deuterium, F, C₁₋₃ alkyl, C₁₋₃ haloalkyl, or C₁₋₄ alkoxyl;
s=0-3;
R^{c1} and R^{d1} are independently H, deuterium, Halo, C₁₋₃ alkyl, C₁₋₃ haloalkyl, or C₁₋₄ alkoxyl;
R^{e3} is H, —C(O)R^{f}, —CH₂—O—P(O)(OR^{g})₂, or —P(O)(OR^{g})₂; wherein Rand R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
R¹ is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}, —(CR^{1a}R^{1b})₁₋₅-A-(CR^{1a}R^{1b})₁₋₅— wherein A is O, S, or NR^{1c}—(CR^{1a}R^{b})₁₋₅-A-(CR^{1a}R^{b})₁₋₅-A- wherein A is O, S, or NR^{c1}, —(CR^{1a}R^{1b})₁₋₅—(CR^{1a}=CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b} groups)-, -(3-11 membered cycloalkyl optionally substituted with 0-6 R^{1a} and/or R^{1b} groups)-(CR^{1a}R^{1b})₁₋₅—, -(3-11 membered heterocyclyl optionally substituted with 0-6 R^{1a} and/or R^{1b} groups)-(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-(3-11 membered heterocyclyl optionally substituted with 0-6 R^{1a} and/or R^{1b} groups)-A- wherein A is O, S, or NR^{1c}, —(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c}, —(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}, —(CR^{1a}R^{1b})₁₋₅-A-(CR^{1a}R^{1b})₁₋₅-A- wherein A is O, S, or NR^{1c}—(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CO) wherein A is O, S, or NR^{1c}, —(CR^{1a}R^{1b})₁₋₅—(CR^{1a}═CR^{1b})—(CR^{1a}R^{1b})₁₋₅-A-(CO)— wherein A is O, S, or NR^{1c}, —(CR^{1a}R^{1b})₁₋₅—(C≡C)—(CR^{1a}R^{1b})₁₋₅-A-(CO)— wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}, —(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}-(3-11 membered cycloalkyl optionally substituted with 0-6 R^{1a} and/or R^{1b} groups)-(CR^{1a}R^{1b})₁₋₅-, or -(3-11 membered heterocyclyl optionally substituted with 0-6 R^{1a} and/or R^{1b} groups)-(CR^{1a}R^{1b})₁₋₅; or R^{j} is -(3-11 membered heterocyclyl optionally substituted with 0-6 R^{1a} and/or R^{1b} groups)-(CO)—(CR^{1a}R^{1b})₁₋₅-A- wherein A is O, S, or NR^{c}; -(3-11 membered heterocyclyl optionally substituted with 0-6 R^{1a} and/or R^{1b} groups)-(CO)-A-(CR^{1a}R^{1b})₁₋₅— wherein A is O, S, or NR^{c}; -A-(3-11 membered heterocyclyl optionally substituted with 0-6 R^{1a} and/or R^{1b} groups)-(CR^{1a}R^{1b})₁₋₅-A- wherein each A is independently O, S, or NR^{c}; -(3-11 membered heterocyclyl optionally substituted with 0-6 R^{1a} and/or R^{1b} groups)-(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}; -(3-11 membered heterocyclyl optionally substituted with 0-6 R^{1a} and/or R^{1b} groups)-(3-11 membered heterocyclyl optionally substituted with 0-6 R^{1a} and/or R^{1b} groups)-(CR^{1a}R^{1b})₁₋₅-A- wherein A is O, S, or NR^{c}; -(3-11 membered heterocyclyl optionally substituted with 0-6 R^{1a} and/or R^{1b} groups)-A-(CR^{1a}R^{1b})₁₋₅-A- wherein each A is independently O, S, or NR^{c}; -(heteroaryl optionally substituted with 0-4 R^{1a} and/or R^{1b} groups)-A-(CR^{1a}R^{1b})₁₋₅— wherein A is O, S, or NR^{c}; -(3-11 membered heterocyclyl optionally substituted with 0-6 R^{1a} and/or R^{1b} groups)-(CR^{1a}R^{1b})₁₋₅-A- wherein A is O, S, or NR^{c}; -(3-11 membered cycloalkyl optionally substituted with 0-6 R^{1a} and/or R^{1b} groups)-(CO)—(CR^{1a}R^{1b})₁₋₅-A- wherein A is O, S, or NR^{1c}; -(3-11 membered cycloalkyl optionally substituted with 0-6 R^{1a} and/or R^{1b} groups)-(CO)-A-(CR^{1a}R^{1b})₁₋₅— wherein A is O, S, or NR^{1c}; or —(CO)-(3-11 membered cycloalkyl optionally substituted with 0-6 R^{1a} and/or R^{1b} groups)-(CR^{1a}R^{1b})₁₋₅-A- wherein A is O, S, or NR^{1c};
R⁴ is H, optionally substituted alkyl, optionally substituted C₁-C₆alkyl, or —CH₃;
R⁷ is optionally substituted alkyl, preferably optionally substituted C₁-C₆alkyl, and more preferably C₁-C₆alkyl; and
R⁹ is H, deuterium, halo, —CN, —OH, —NO₂, —NR^{e1}R^{e2}, —OR^{e1}, —CONR^{e1}R^{e2}, —NR^{e1}COR^{e2}, —SO₂NR^{e1}R^{e2}, —NR^{e1}SO₂R^{e2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R^{1a}, R^{1b}, R^{1c}, and R^{1e} are each independently, —H, deuterium, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1c} groups; and
each R^{e1} and R^{e2} is independently H, deuterium, or optionally substituted alkyl, or R^{e1} and R^{e2} together with the nitrogen atom to which they are attached form a 4-7 membered heterocyclyl.

84. The Degrader-Linker compound of any one of claims 80 to 83, or a pharmaceutically acceptable salt thereof, wherein D has a structure of: 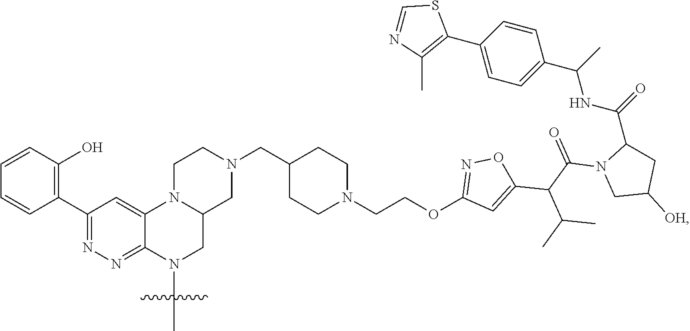 [SMILES: *N1CC2CN(CC3CCN(CCOc4cc(C(C(=O)N5CC(O)CC5C(=O)NC(C)c5ccc(-c6scnc6C)cc5)C(C)C)on4)CC3)CCN2c2cc(-c3ccccc3O)nnc21]
wherein the wavy line indicates the point of covalent attachment to L′.

85. The Degrader-Linker compound of any one of claims 80 to 83, or a pharmaceutically acceptable salt thereof, wherein D has a structure of: 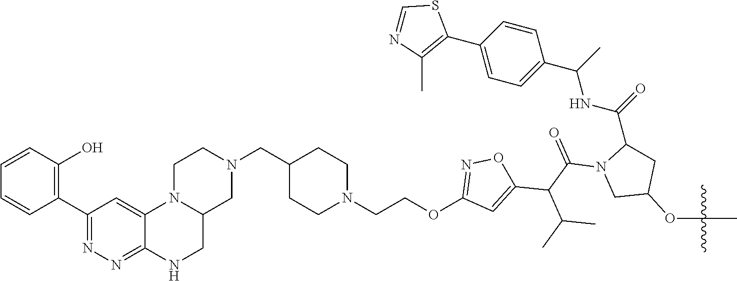 [SMILES: *OC1CC(C(=O)NC(C)c2ccc(-c3scnc3C)cc2)N(C(=O)C(c2cc(OCCN3CCC(CN4CCN5c6cc(-c7ccccc7O)nnc6NCC5C4)CC3)no2)C(C)C)C1]
wherein the wavy line indicates the point of covalent attachment to L′.

86. The Degrader-Linker compound of any one of claims 80 to 83, or a pharmaceutically acceptable salt thereof, wherein D has a structure of: 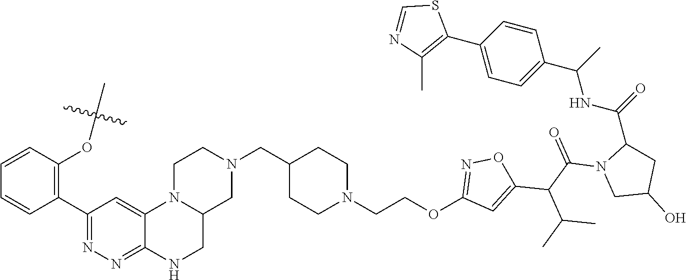 [SMILES: *Oc1ccccc1-c1cc2c(nn1)NCC1CN(CC3CCN(CCOc4cc(C(C(=O)N5CC(O)CC5C(=O)NC(C)c5ccc(-c6scnc6C)cc5)C(C)C)on4)CC3)CCN21]
wherein the wavy line indicates the point of covalent attachment to L′.

87. The Degrader-Linker compound of claim 80 or 81, or a pharmaceutically acceptable salt thereof, wherein D has a structure of: 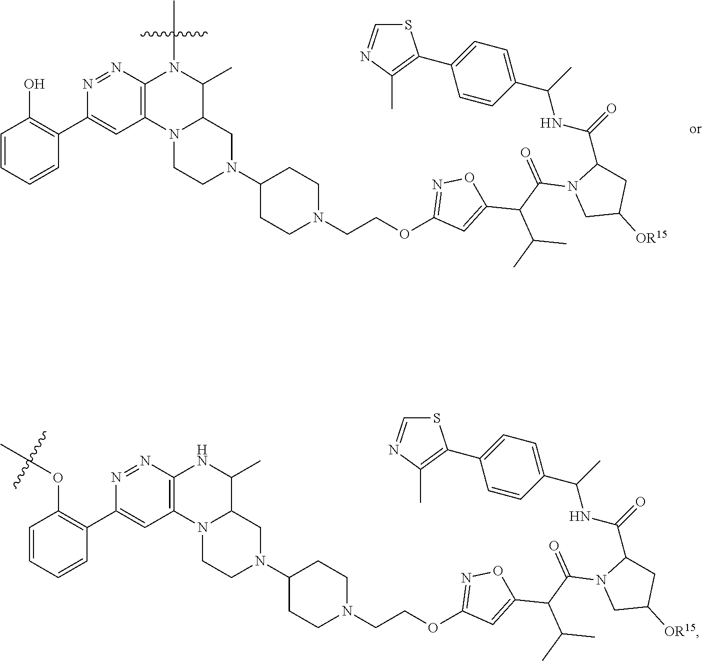
wherein R¹⁵ is hydrogen or —PO₃H₂ and the wavy line indicates the point of covalent attachment to L′.

88. The Degrader-Linker compound of claim 87, or a pharmaceutically acceptable salt thereof, wherein D has a structure of:

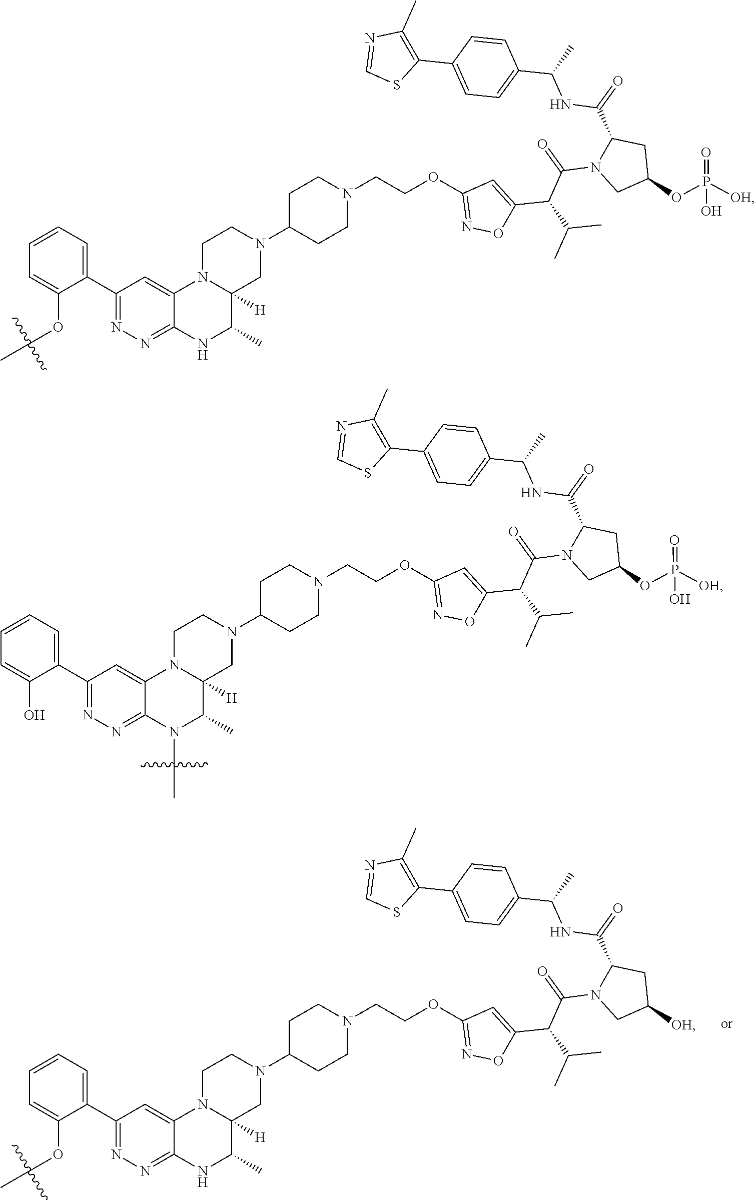 [SMILES: *Oc1ccccc1-c1cc2c(nn1)N[C@@H](C)[C@@H]1CN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)CCN21 ; *N1c2nnc(-c3ccccc3O)cc2N2CCN(C3CCN(CCOc4cc([C@H](C(=O)N5C[C@H](OP(=O)(O)O)C[C@H]5C(=O)N[C@@H](C)c5ccc(-c6scnc6C)cc5)C(C)C)on4)CC3)C[C@H]2[C@@H]1C ; *Oc1ccccc1-c1cc2c(nn1)N[C@@H](C)[C@@H]1CN(C3CCN(CCOc4cc([C@H](C(=O)N5C[C@H](O)C[C@H]5C(=O)N[C@@H](C)c5ccc(-c6scnc6C)cc5)C(C)C)on4)CC3)CCN21 ; "or"]

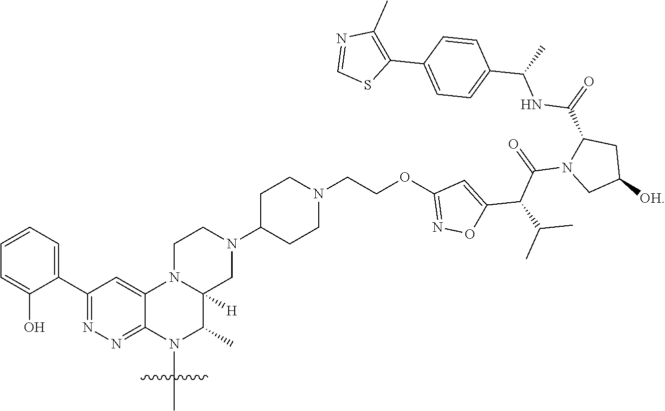 [SMILES: *N1c2nnc(-c3ccccc3O)cc2N2CCN(C3CCN(CCOc4cc([C@H](C(=O)N5C[C@H](O)C[C@H]5C(=O)N[C@@H](C)c5ccc(-c6scnc6C)cc5)C(C)C)on4)CC3)C[C@H]2[C@@H]1C]

89. The Degrader-Linker compound of any one of claims 76 to 79, or a pharmaceutically acceptable salt thereof, wherein ULM binds Cereblon E3 Ubiquitin Ligase.

90. The Degrader-Linker compound of claim 89, or a pharmaceutically acceptable salt thereof, wherein ULM is a moiety having the Formula ULM-II-CRBN: 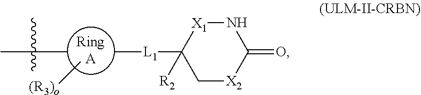
wherein: [symbol]
is a point of attachment to PTM;
Ring A is a monocyclic, bicyclic or tricyclic aryl, heteroaryl or heterocycle group,
L₁ is a bond, —O—, —S—, —NR^{a}—, —C(R^{a})₂—, or —C(O)NR^{a}—;
X₁ is a bond, —C(O)—, —C(S)—, —CH₂—, —CHCF₃—, SO₂—, —S(O), P(O)R^{b}—or —P(O)OR^{b}—;
X₂ is —C(R^{a})₂—, —NR^{a}— or —S—;
R₂ is H, deuterium, optionally substituted C₁₋₄ alkyl, C₁₋₄ alkoxyl, C₁₋₄ haloalkyl, —CN, —OR^{a}, —OR^{b} or —SR^{b};
each R₃ is independently H, deuterium, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{e2}R^{d} —NR^{a}R^{c2}, —C(O)R^{b}, —OC(O)R^{a}, —C(O)OR^{a}, —C(O)NR^{e2}R^{d}, —S(O)R^{b}, —S(O)₂NR^{e2}R^{d}, —S(O)(═NR^{b})R^{b}, —SF₅, —P(O)R^{b}R^{b}, —P(O)(OR^{b})(OR^{b}), —B(OR^{d})(OR^{c2}) or —S(O)₂R^{b};
each R^{a} is independently H, deuterium, —C(O)R^{b}, —C(O)OR^{c2}, —C(O)NR^{e2}R^{d}, —C(═NR^{b})NR^{b}R^{c2}, —C(═NOR^{b})NR^{b}R^{c2}, —C(═NCN)NR^{b}R^{c2}, —P(OR^{c2})₂, —P(O)R^{e2}R^{b}, —P(O)OR^{e2}OR^{b}, —S(O)R^{b}, —S(O)NR^{e2}R^{d}, —S(O)₂R^{b}, —S(O)₂NR^{e2}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, deuterium, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c2} or R^{d} is independently H, deuterium, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c2} and R^{d}, together with the atom to which they are both attached, form a monocyclic or multicyclic heterocycloalkyl, or a monocyclic or multicyclic heterocyclo-alkenyl group; and
o is 1, 2, 3, 4, or 5.

91. The Degrader-Linker compound of claim 89 or 90, or a pharmaceutically acceptable salt thereof, wherein ULM-II-CRBN is a compound of formula: 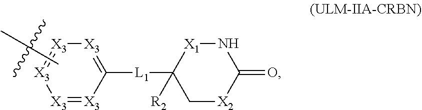
wherein each X₃ is independently N, N-oxide or CR₃ and at least one X₃ is N or N-oxide;
wherein [symbol] is a point of attachment to PTM; or 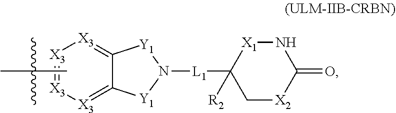
wherein each X₃ is independently N, N-oxide or CR₃;
wherein each Y₁ is independently —C(O)— or —C(R^{a})₂—and at least one Y₁ is —C(O)—; and
wherein [symbol] is a point of attachment to PTM; or 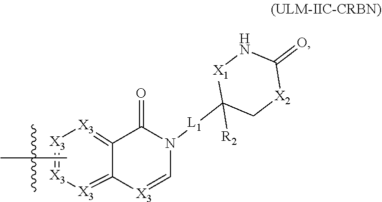
wherein each X₃ is independently N, N-oxide or CR₃ and wherein [symbol] is a point of attachment to PTM; or 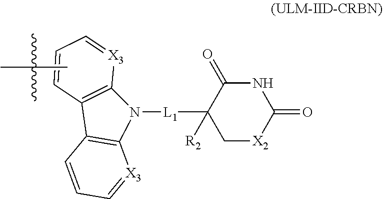
wherein each X₃ is independently N, N-oxide or CR₃ and wherein [symbol] is a point of attachment to PTM.

92. The Degrader-Linker compound of any one of claims 89 to 91, or a pharmaceutically acceptable salt thereof, wherein D has a structure of: 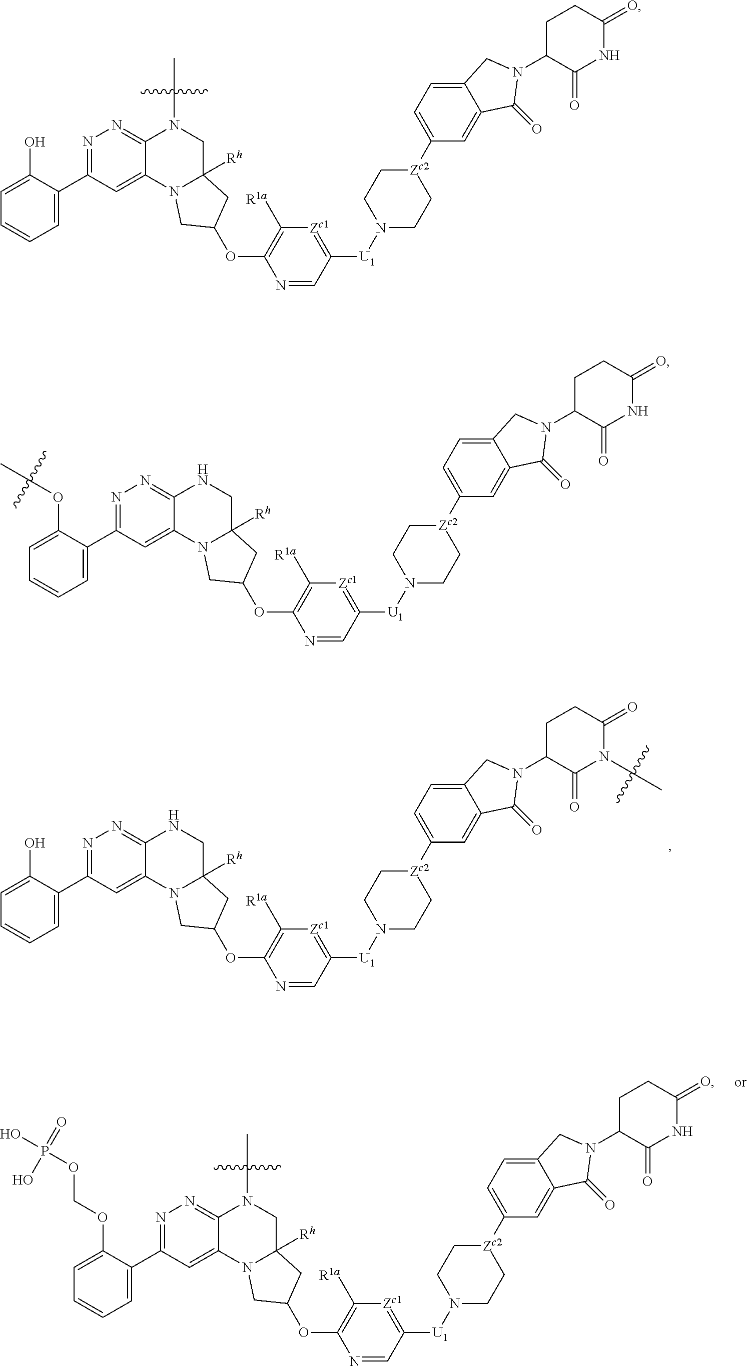 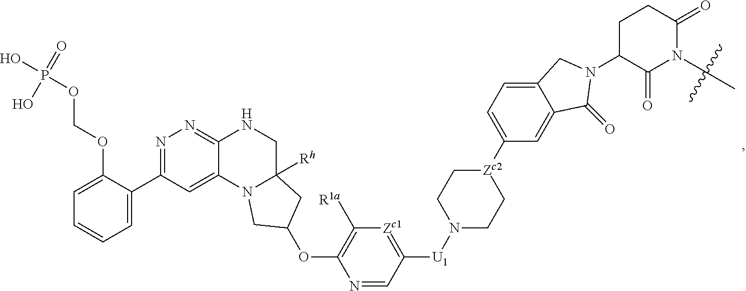
wherein
the wavy line indicates the point of covalent attachment to L′;
R^{h} is methyl or ethyl;
R^{1a} is hydrogen, C₁₋₃ alkyl, or halo;
each of Z^{c1} and Z^{c2} is independently CH or N; and
U₁ is —CH₂— or —C(O)—.

93. The Degrader-Linker compound of claim 92, or a pharmaceutically acceptable salt thereof, wherein D has a structure of: 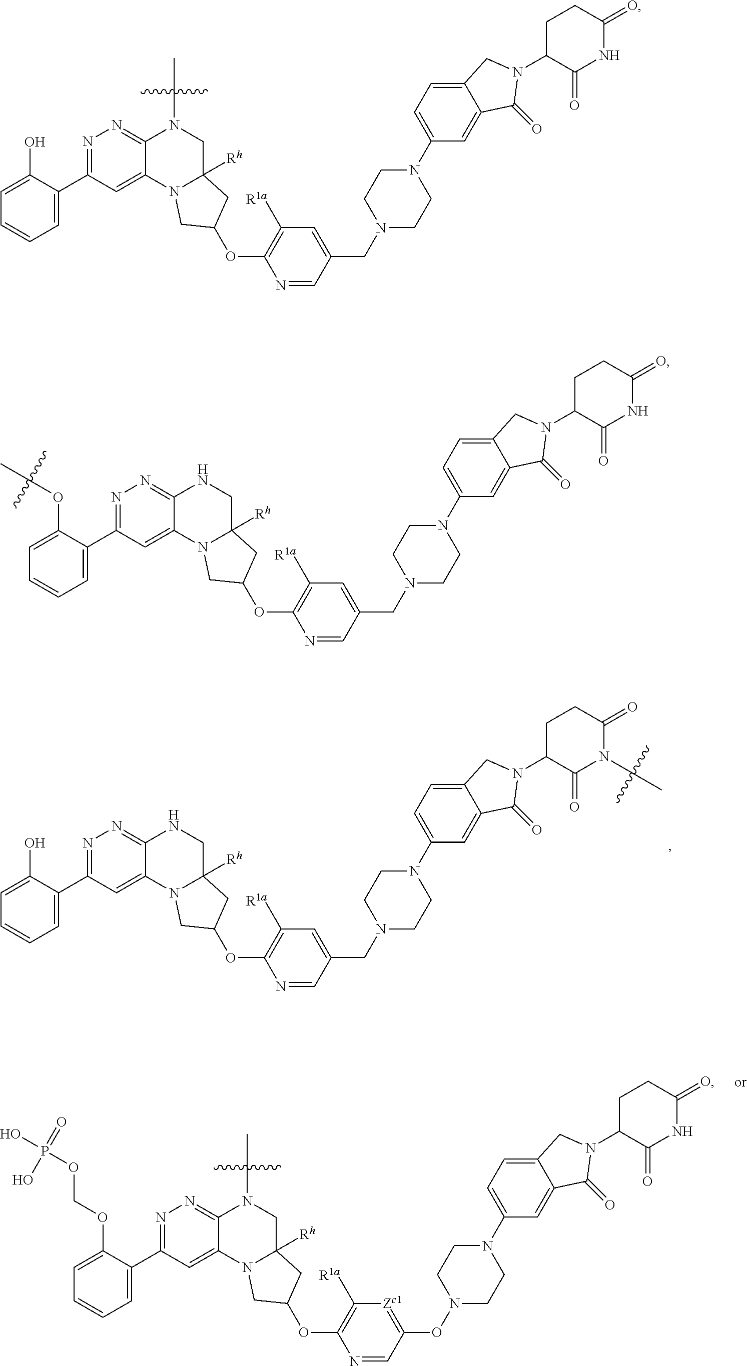 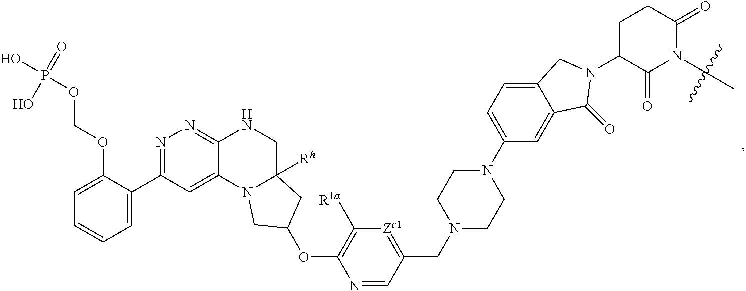
wherein R^{h} is methyl or ethyl; R^{1a} is hydrogen, methyl, or fluorine, and the wavy line indicates the point of covalent attachment to L′.

94. The Degrader-Linker compound of any one of claims 76 to 93, or a pharmaceutically acceptable salt thereof, having the formula of:

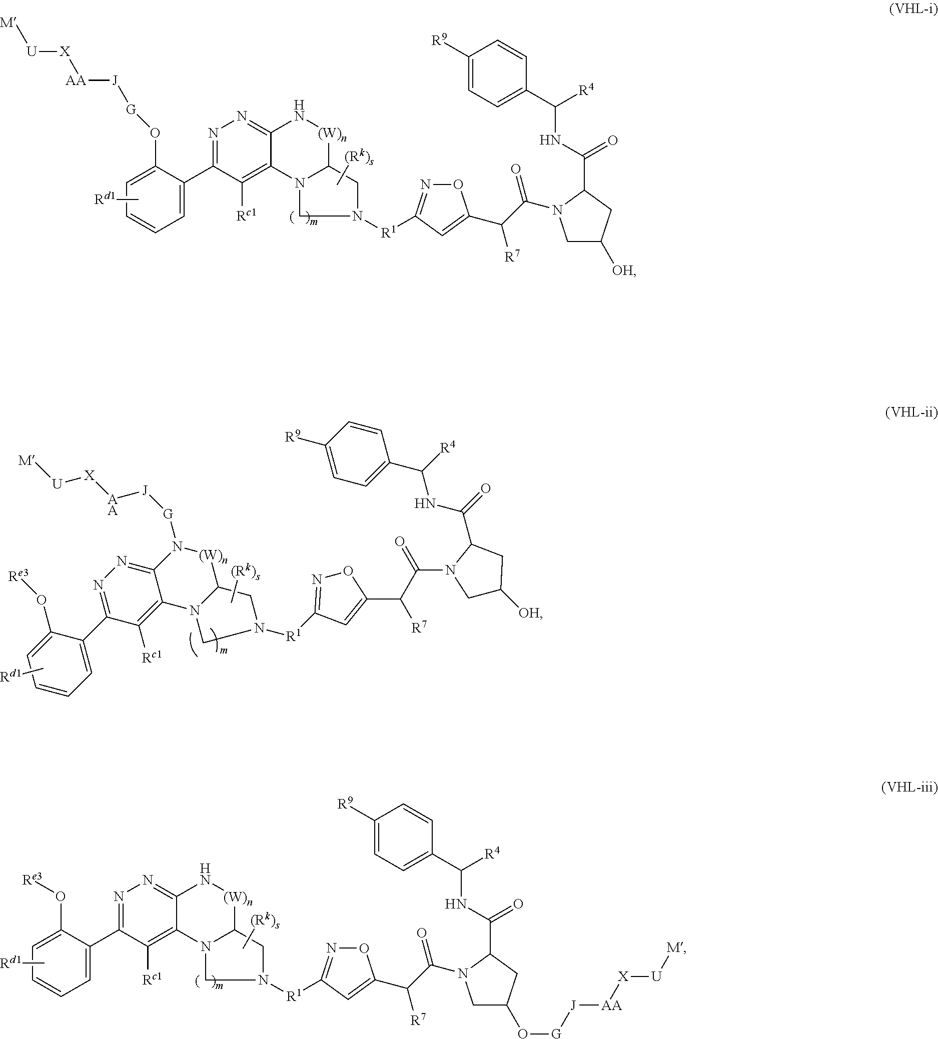

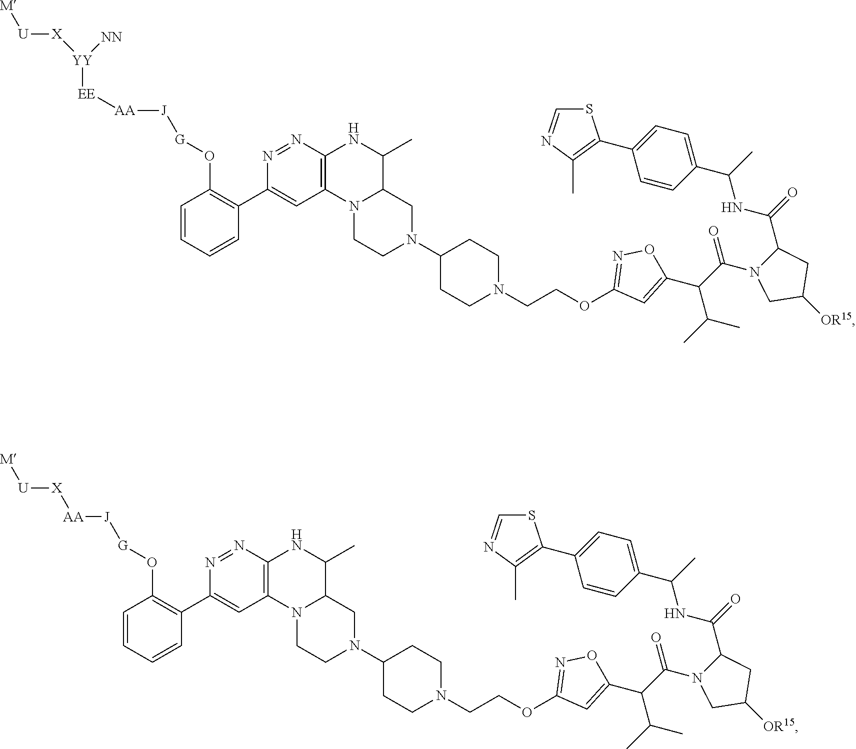

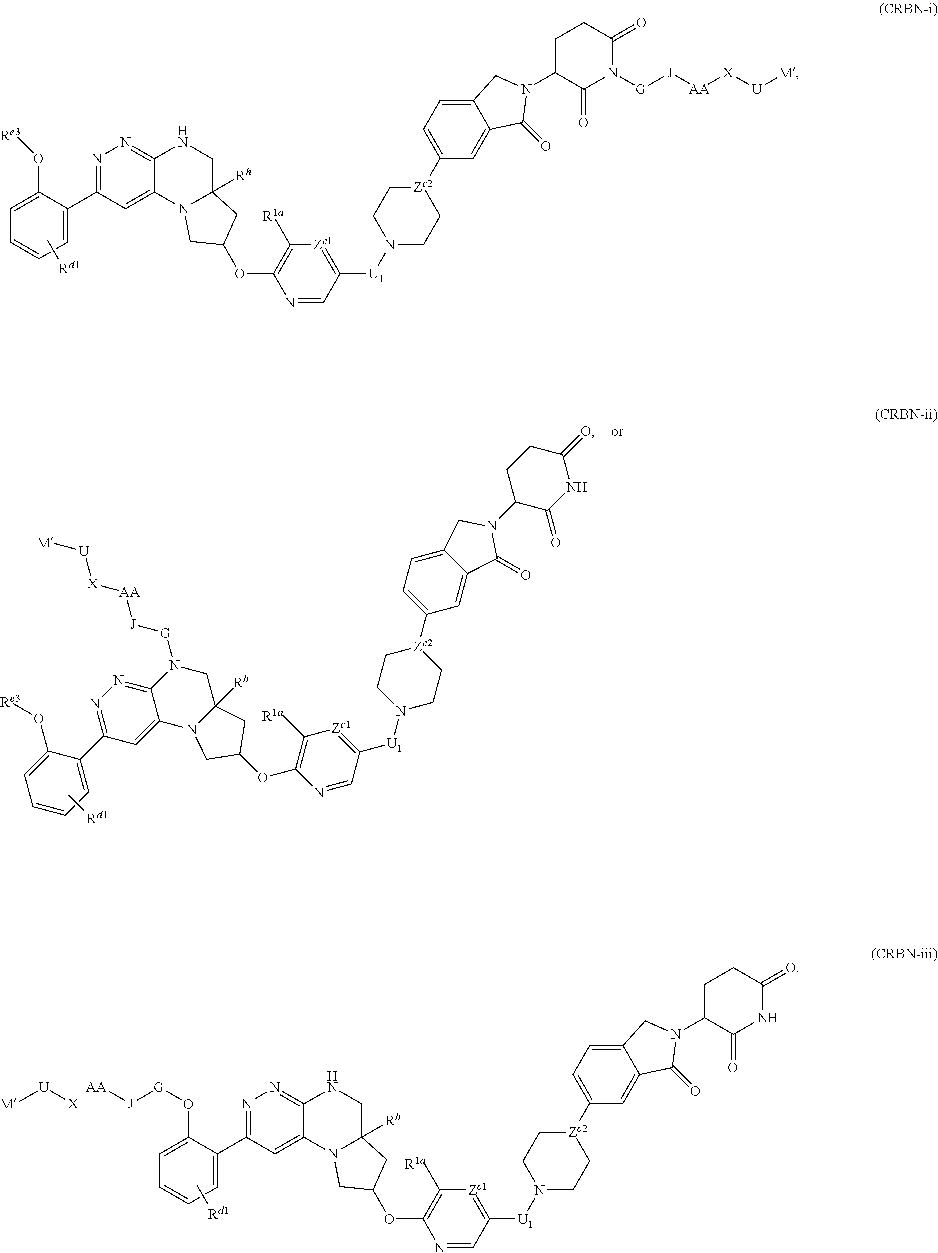

95. The Degrader-Linker compound of any one of claims 76 to 94, or a pharmaceutically acceptable salt thereof, wherein
AA is present;
subscript c is an integer ranging between 1 and 12; and
each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine.

96. The Degrader-Linker compound of claim 94 or 95, or a pharmaceutically acceptable salt thereof, represented by the structure of: 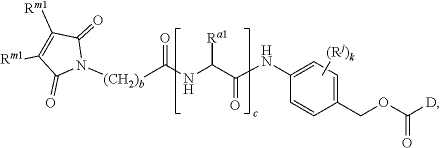
wherein
each R^{m1} is hydrogen, halogen, or —S-Ph;
subscript b is an integer ranging from 1 to 5;
subscript c is an integer ranging from 1 to 5;
each R^{a1} is independently the side chain of valine, glutamic acid, tryptophan, glycine, citrulline, lysine, alanine, or phenylalanine;
each R^{j} is independently fluorine, chlorine, methyl, or —C(O)NHCH₃; and
subscript k is an integer ranging from 0 to 4.

97. The Degrader-Linker compound of claim 96, or a pharmaceutically acceptable salt thereof, represented by the structure of: 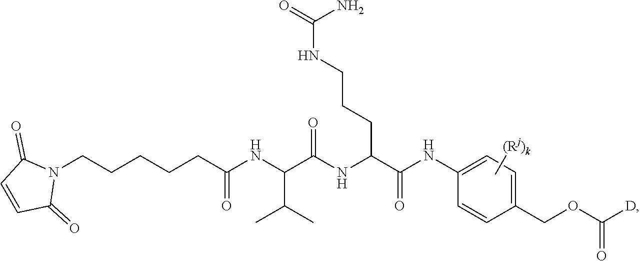
wherein each R^{j} is methyl, —F, —Cl, or —C(O)NHCH₃, and subscript k is 0 or 1.

98. The Degrader-Linker compound of claim 96, or a pharmaceutically acceptable salt thereof, represented by the structure of:

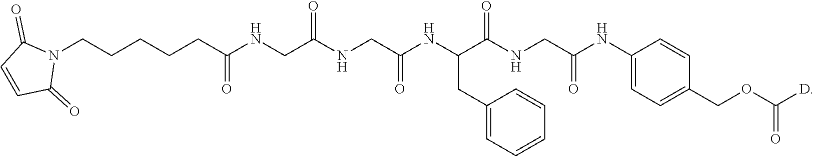

99. The Degrader-Linker compound of claim 98, or a pharmaceutically acceptable salt thereof, represented by the structure of:

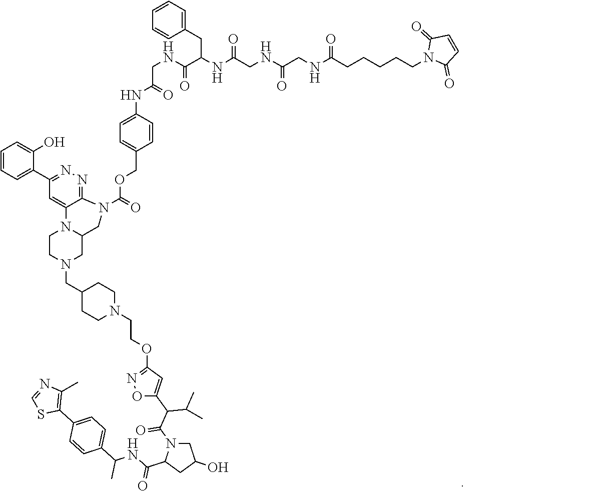 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6ccc(NC(=O)CNC(=O)C(Cc7ccccc7)NC(=O)CNC(=O)CNC(=O)CCCCCN7C(=O)C=CC7=O)cc6)CC5C4)CC3)no2)C(C)C)cc1]

100. The Degrader-Linker compound of claim 94 or 95, or a pharmaceutically acceptable salt thereof, represented by the structure of: 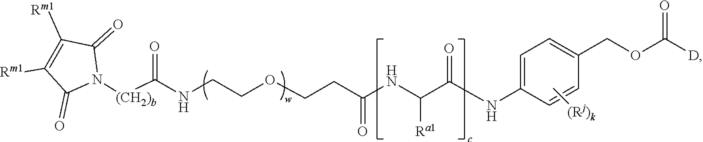
each R^{m1} is hydrogen, halogen, or —S-Ph;
subscript b is an integer ranging from 1 to 5;
subscript w is an integer ranging from 1 to 8;
subscript c is an integer ranging from 1 to 3;
each R^{a1} is independently the side chain of valine, glutamic acid, tryptophan, glycine, citrulline, lysine, alanine, or phenylalanine;
each R^{j} is independently fluorine, chlorine, methyl, or —C(O)NHCH₃; and
subscript k is an integer ranging from 0 to 4.

101. The Degrader-Linker compound of claim 100, or a pharmaceutically acceptable salt thereof, represented by the structure of: 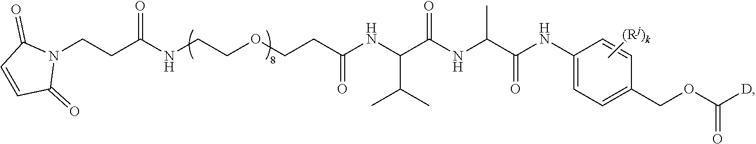
wherein R^{j} is methyl, —F, —Cl, or —C(O)NHCH₃, and subscript k is 0 or 1.

102. The Degrader-Linker compound of claim 100, or a pharmaceutically acceptable salt thereof, represented by the structure of:

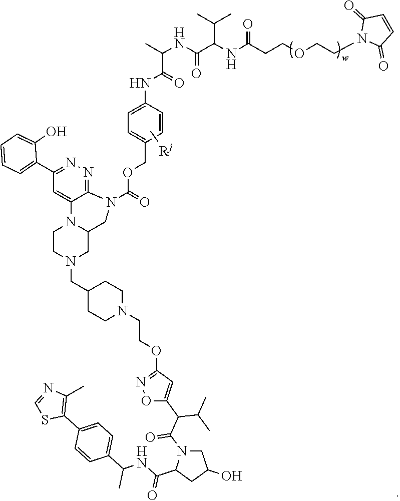

103. The Degrader-Linker compound of claim 94 or 95, or a pharmaceutically acceptable salt thereof, represented by the structure of: 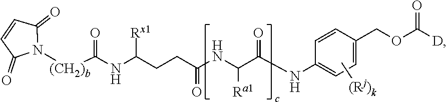
wherein
subscript b is an integer ranging from 1 to 5;
subscript c is an integer ranging from 1 to 3;
R^{x1} is hydrogen, —COOH, —C(O)NHCH₃, or —(C(O)NHCH₂)ₓ(CH₂OCH₂)_{y}R^{x2} wherein R^{x2} is —CH₂NHC(O)CH₃, —CH₂NH₂, or —CH₂C(O)OR^{x2a}, wherein R^{x2a} is hydrogen or C₁-C₆ alkyl;
each R^{a1} is independently the side chain of valine, glutamic acid, tryptophan, glycine, citrulline, lysine, alanine, or phenylalanine;
each R^{j} is independently fluorine, chlorine, methyl, or —C(O)NHCH₃; and
subscript k is an integer ranging from 0 to 4.

104. The Degrader-Linker compound of claim 103, or a pharmaceutically acceptable salt thereof, wherein R^{x1} is

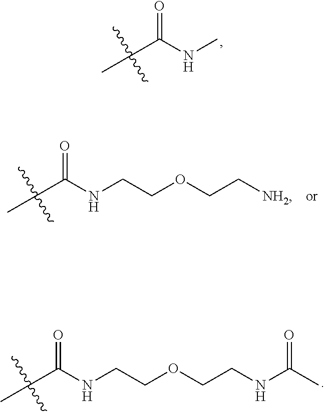

105. The Degrader-Linker compound of claim 94 or 95, or a pharmaceutically acceptable salt thereof, represented by the structure of: 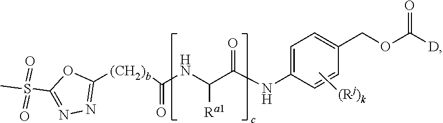
wherein
subscript b is an integer ranging from 1 to 5;
subscript c is 1 or 2;
each R^{a1} is independently the side chain of valine or citrulline;
each R^{j} is independently fluorine, chlorine, methyl, or —C(O)NHCH₃; and
subscript k is an integer ranging from 0 to 4.

106. The Degrader-Linker compound of claim 105, or a pharmaceutically acceptable salt thereof, represented by the structure of:

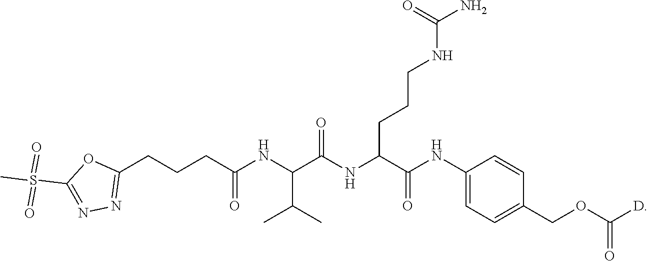

107. The Degrader-Linker compound of claim 105 or 106, or a pharmaceutically acceptable salt thereof, represented by the structure of:

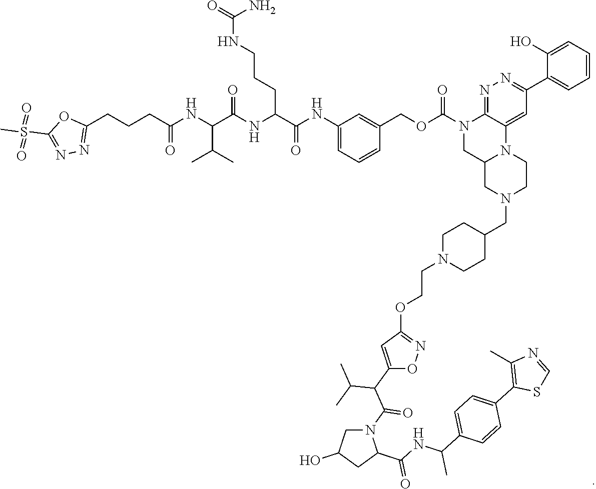 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6cccc(NC(=O)C(CCCNC(N)=O)NC(=O)C(NC(=O)CCCc7nnc(S(C)(=O)=O)o7)C(C)C)c6)CC5C4)CC3)no2)C(C)C)cc1]

108. The Degrader-Linker compound of claim 94 or 95, or a pharmaceutically acceptable salt thereof, represented by the structure of: 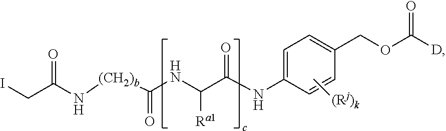
wherein
subscript b is an integer ranging from 1 to 5;
subscript c is 1 or 2;
each R^{a1} is independently the side chain of valine or citrulline;
each R^{j} is independently fluorine, chlorine, methyl, or —C(O)NHCH₃; and
subscript k is an integer ranging from 0 to 4.

109. The Degrader-Linker compound of claim 108, or a pharmaceutically acceptable salt thereof, represented by the structure of:

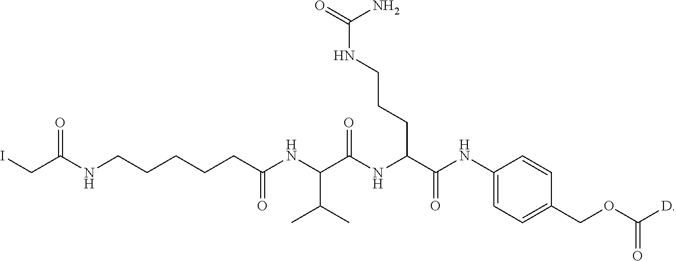

110. The Degrader-Linker compound of claim 108, or a pharmaceutically acceptable salt thereof, represented by the structure of:

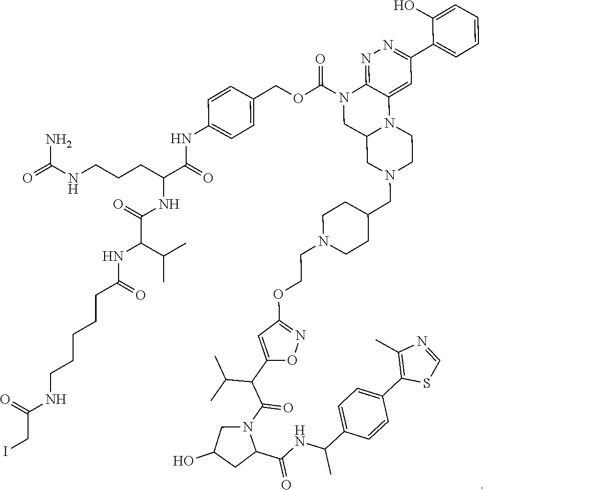 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6ccc(NC(=O)C(CCCNC(N)=O)NC(=O)C(NC(=O)CCCCCNC(=O)CI)C(C)C)cc6)CC5C4)CC3)no2)C(C)C)cc1 ; "."]

111. The Degrader-Linker compound of claim 94 or 95, or a pharmaceutically acceptable salt thereof, represented by the structure of: 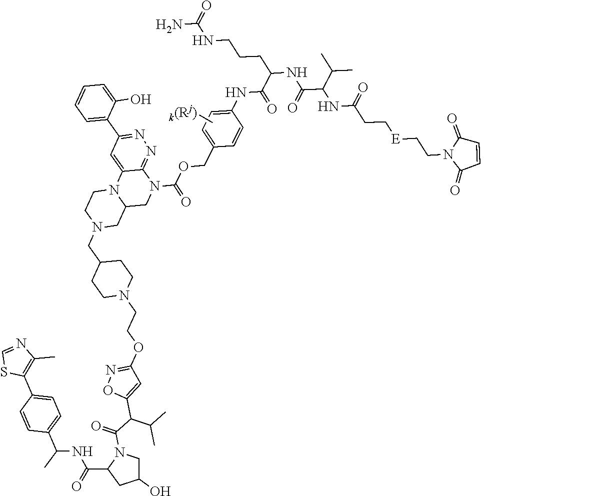
wherein E is —CH₂— or —O—; R^{j} is methyl, —F, —Cl, or —C(O)NHCH₃; and subscript k is 0 or 1.

112. The Degrader-Linker compound of claim 94 or 95, or a pharmaceutically acceptable salt thereof, represented by the structure of: 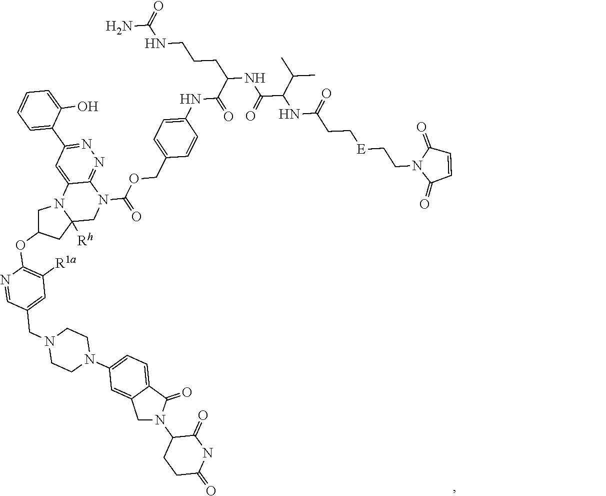
wherein E is —CH₂— or —O—; R^{h} is methyl or ethyl; and R^{1a} is hydrogen or methyl.

113. The Degrader-Linker compound of claim 94 or 95, or a pharmaceutically acceptable salt thereof, represented by the structure of: 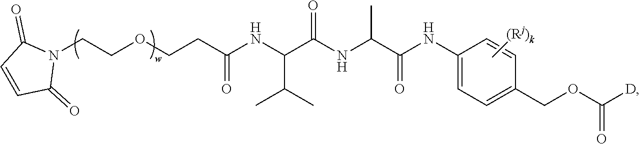
wherein
subscript w is 1 or 2;
each R^{j} is independently fluorine, chlorine, methyl, or —C(O)NHCH₃; and
subscript k is an integer ranging from 0 to 4.

114. The Degrader-Linker compound of claims 94 or 95, or a pharmaceutically acceptable salt thereof, represented by the structure of:

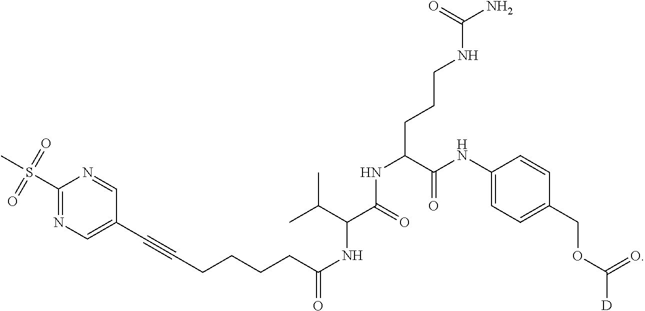 [SMILES: [2H]C(=O)OCc1ccc(NC(=O)C(CCCNC(N)=O)NC(=O)C(NC(=O)CCCCC#Cc2cnc(S(C)(=O)=O)nc2)C(C)C)cc1]

115. The Degrader-Linker compound of claim 94 or 95, or a pharmaceutically acceptable salt thereof, represented by the structure of: 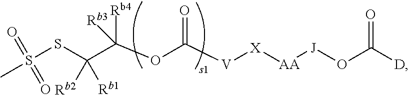
wherein
R^{b1} and R^{b2} are each independently hydrogen, C₁-C₆ alkyl, or both R^{b1} and R^{b2}, together with the carbon to which they are attached, comprise a C₄-C₆ cycloalkyl or a C₄-C₆ heterocycloalkyl;
R^{b3} and R^{b4} are each independently hydrogen or C₁-C₆ alkyl; and
subscript s1 is 0.

116. The Degrader-Linker compound of claim 115, or a pharmaceutically acceptable salt thereof, represented by the structure of:

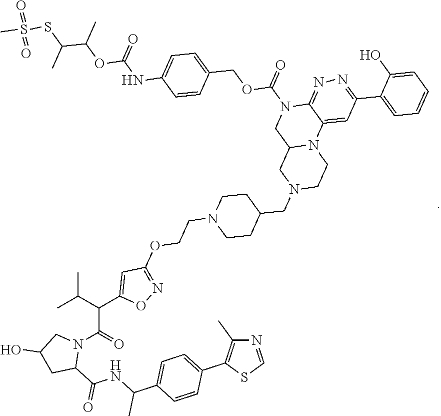 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCc6ccc(NC(=O)OC(C)C(C)SS(C)(=O)=O)cc6)CC5C4)CC3)no2)C(C)C)cc1 ; "."]

117. The Degrader-Linker compound of claim 116, or a pharmaceutically acceptable salt thereof, wherein U, X, and AA are each absent.

118. The Degrader-Linker compound of claim 117, or a pharmaceutically acceptable salt thereof, represented by the structure of: 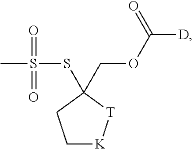
wherein T is absent or is —CH₂CH₂—and K is —CH₂— or —NH₂—.

119. The Degrader-Linker compound of claim 118, or a pharmaceutically acceptable salt thereof, represented by the structure of:

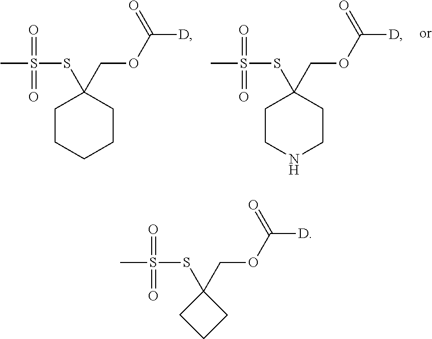

120. The Degrader-Linker compound of claim 119, or a pharmaceutically acceptable salt thereof, represented by the structure of:

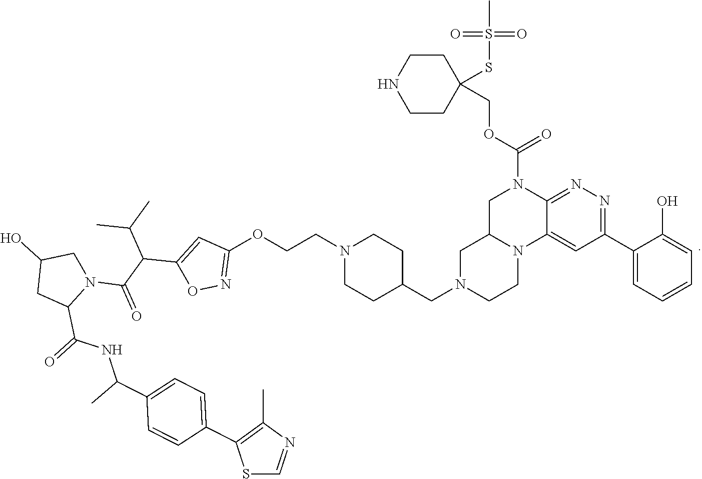 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OCC6(SS(C)(=O)=O)CCNCC6)CC5C4)CC3)no2)C(C)C)cc1 ; "."]

121. The Degrader-Linker compound of claim 117, or a pharmaceutically acceptable salt thereof, represented by the structure of:

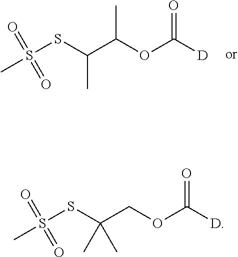

122. The Degrader-Linker compound of claim 121, or a pharmaceutically acceptable salt thereof, represented by the structure of:

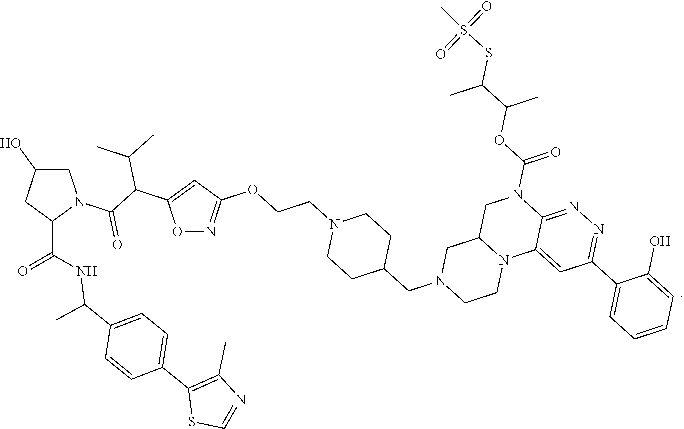 [SMILES: Cc1ncsc1-c1ccc(C(C)NC(=O)C2CC(O)CN2C(=O)C(c2cc(OCCN3CCC(CN4CCN5c6cc(-c7ccccc7O)nnc6N(C(=O)OC(C)C(C)SS(C)(=O)=O)CC5C4)CC3)no2)C(C)C)cc1 ; "."]

123. The Degrader-Linker compound of claim 80 or 81, or a pharmaceutically acceptable salt thereof, represented by the structure of: 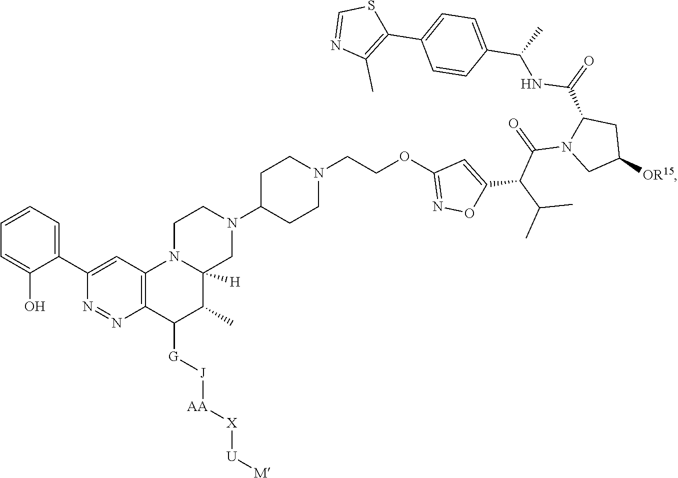
wherein R¹⁵ is hydrogen or —PO₃H₂.

124. The Degrader-Linker compound of claim 123, or a pharmaceutically acceptable salt thereof, represented by the structure of: 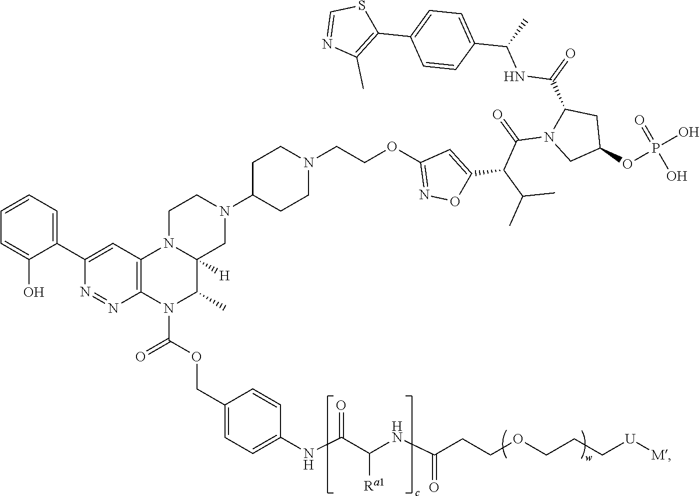
wherein
subscript c is an integer ranging between 1 and 4;
subscript w is an integer ranging between 1 and 8; and
U is absent or is —CH₂—(C═O)—(NH)—.

125. The Degrader-Linker compound of claim 80 or 81, or a pharmaceutically acceptable salt thereof, represented by the structure of: 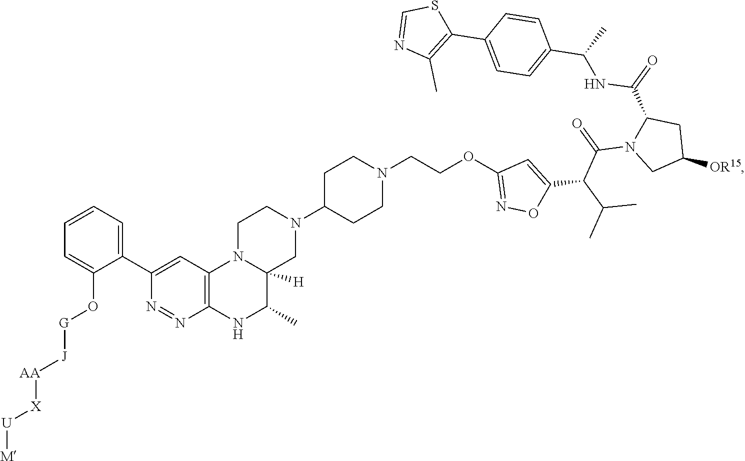
wherein R¹⁵ is hydrogen or —PO₃H₂.

126. The Degrader-Linker compound of claim 125, or a pharmaceutically acceptable salt thereof, represented by the structure of: 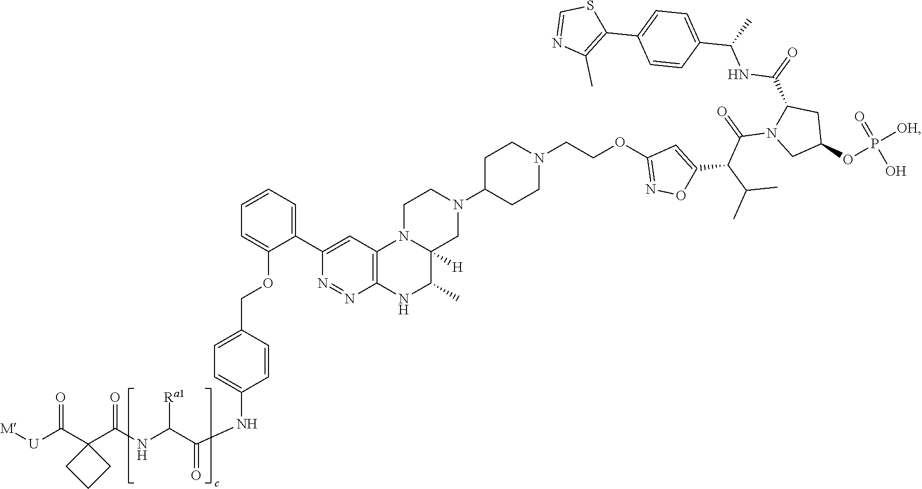
wherein
subscript c is an integer ranging between 1 and 4; and
U is —(CH₂CH₂O)_{w}CH₂CH₂(NH)—, wherein subscript w is an integer ranging from 1 to 4.

127. A Degrader-Linker compound, or a pharmaceutically acceptable salt thereof, represented by the structure of: 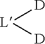
wherein,
L′ is a linker precursor of Formula
each D is independently a degrader compound of Formula (I):
PTM-ULM (I)
wherein,
PTM is a moiety of Formula IA: 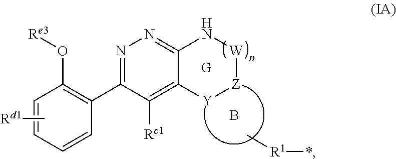
wherein,
R¹ is a covalent bond, or chemical moiety that links PTM and ULM;
* is a point of attachment to ULM;
n=0-3;
each W is independently optionally substituted —CH₂—, —C(O)—, —S(O)—, or —S(O)₂—, wherein when n=2 or 3, only one W is —C(O)—, —S(O)—, or —S(O)₂—, and the other W are —CH₂— or substituted —CH₂—;
R^{c1} and R^{d1} are independently H, deuterium, Halo, C₁₋₃ alkyl, C₁₋₃ haloalkyl, or C₁₋₄ alkoxyl;
R^{e3} is hydrogen, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃-8 substituted cyclcoalkyl, C₃₋₈ heterocyclcoalkyl, or C₃₋₈ substituted heterocyclcoalkyl;
Z and Y are each independently N; CR^{h} wherein R^{h}=H, C₁₋₃ alkyl, or absent; or, if R¹ is attached to Z, then Z is C and Y is N or CR^{h} wherein R^{h} is H or C₁₋₃ alkyl; or if R¹ is attached to Y, then Y is C and Z is N or CR^{h} wherein R^{h} is H or C₁₋₃ alkyl;
B is an optionally substituted 5-7 membered cycloalkyl ring, an optionally substituted 5-7 membered heteroaryl ring, or an optionally substituted 5-7 membered heterocyclic ring, wherein ring B is fused to ring G through Y and Z; and
ULM is a small molecule E3 Ubiquitin Ligase binding moiety that binds a Von Hippel-Lindau E3 Ubiquitin Ligase or a Cereblon E3 Ubiquitin Ligase.

128. The Degrader-Linker compound of claim 127, or a pharmaceutically acceptable salt thereof, wherein L′ is a linker precursor of Formula (iii): 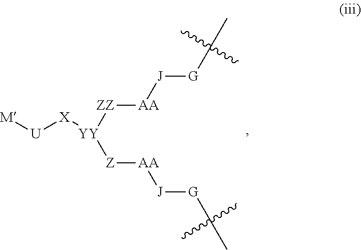
wherein
M′ is selected from the group consisting of: 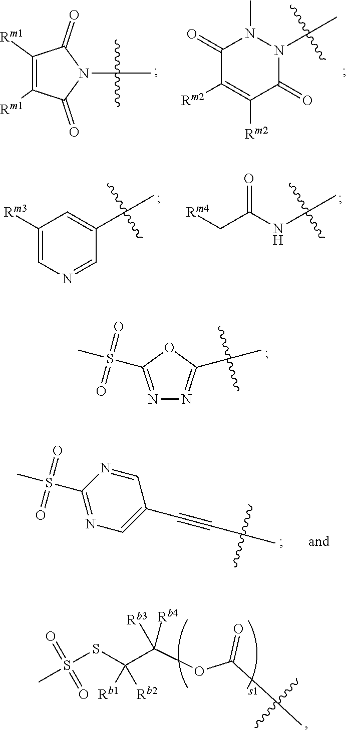
wherein
each R^{m1} and R^{m2} is independently hydrogen, halogen, or —S-Ph;
R^{m3} and R^{m4} are each halogen;
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1;
wherein the wavy line of M′ indicates the point of covalent attachment to the remainder of the structure of L′ and the wavy line of Formula (iii) indicates the point of covalent attachment to the degrader compound (D); and
U is absent or is —(CH₂)_{b}(R^{v1})ₛₛ(C═O)ᵤ(NH)ᵥ— wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—OC(O)—, wherein R^{x5} is hydrogen or C₁-C₆ alkyl and R^{x6} is hydrogen, C₁-C₆ alkyl, or 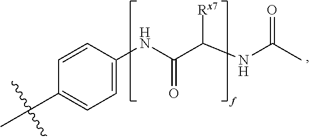
wherein subscript f is an integer ranging from 0 to 4, and each R^{x7} is independently hydrogen, —COOH, —NH₂, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit selected from the group consisting of: 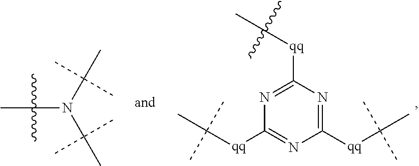
wherein
each qq is independently —N(R^{y1})— or —O—, wherein R^{y1} is hydrogen or C₁-C₆ alkyl;
the wavy line indicates the point of attachment to X, when present, or U, when X is absent, or M′, when X and U are absent; and
each dashed line indicates the point of attachment to ZZ when ZZ is present, or AA when ZZ is absent, or J when AA and ZZ are absent, or G when AA, ZZ, and J are absent;
each ZZ is independently —(CH₂CH₂O)_{w′}CH₂CH₂C(O)—, wherein subscript w′ is an integer ranging from 0 to 16;
each AA is independently absent or has the structure of: 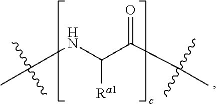
wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of: 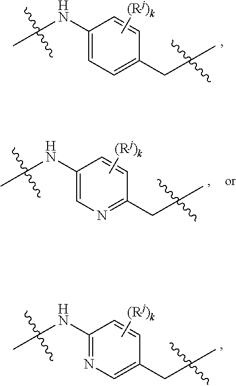
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
each G is independently absent, 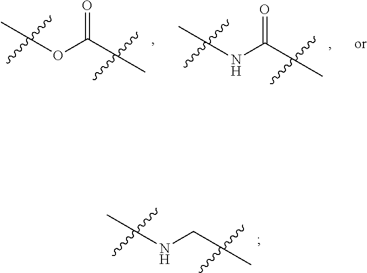
and
wherein the wavy line to each G of Formula (iii) indicates the point of covalent attachment to an independently selected D.

129. The Degrader-Linker compound of claim 128, or a pharmaceutically acceptable salt thereof, wherein L′ has the structure of

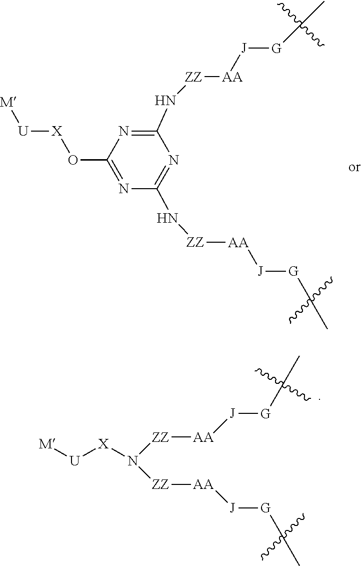

130. The Degrader-Linker compound of claim 128 or 129, or a pharmaceutically acceptable salt thereof, wherein L′ has the structure of 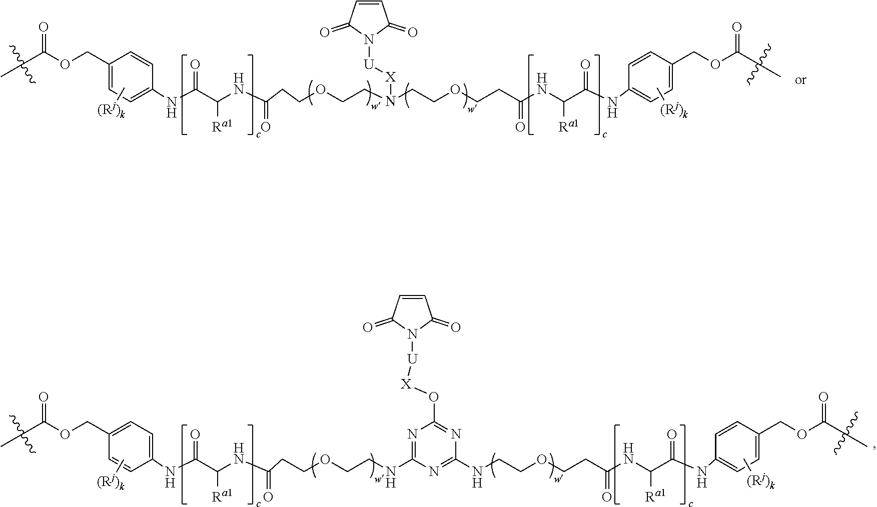
wherein
U is absent or is —(CH₂)_{b}(R^{v1})ₛₛ(C═O)ᵤ(NH)ᵥ—, wherein subscript b is 0, 1, 2, 3, 4, or 5; subscript ss is 0 or 1; subscript u is 0 or 1; subscript v is 0 or 1; R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl;
X is absent or is —(CH₂CH₂O)_{w}CH₂CH₂—(C═O)_{d}—, wherein subscript w is an integer ranging from 0 to 8 and subscript d is 0 or 1;
subscript c is an integer ranging from 1 to 12 and each R^{a1} is independently —COOH, —NH₂, or an independently selected side chain of an amino acid, wherein each amino acid is selected from the group consisting of alanine, valine, glutamic acid, citrulline, tryptophan, glycine, phenylalanine, and lysine;
each R³ is independently fluorine, chlorine, methyl, or —C(O)NHCH₃; and
subscript k is an integer ranging from 0 to 4.

131. The Degrader-Linker compound of any one of claims 128-130, or a pharmaceutically acceptable salt thereof, wherein L′ has the structure of

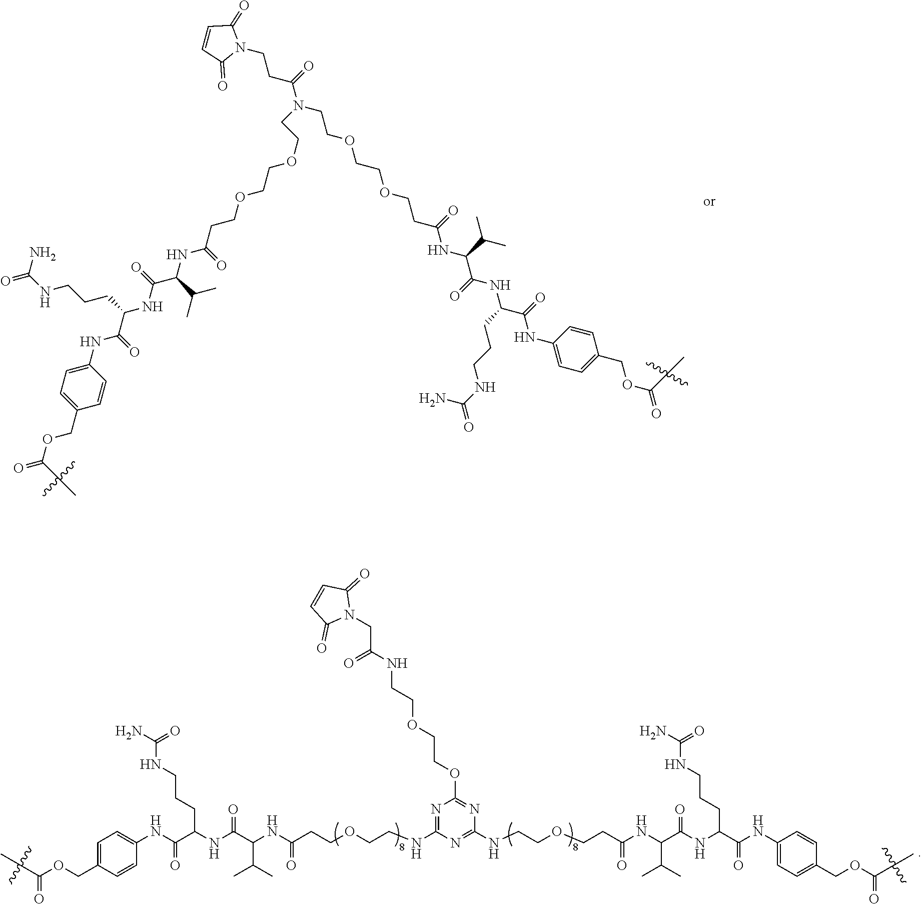

132. The Degrader-Linker compound of claim 127, or a pharmaceutically acceptable salt thereof, wherein L′ is a linker precursor of Formula (iib): 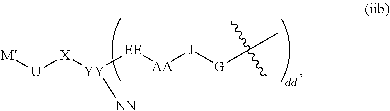
wherein
subscript dd is 2;
M′ is selected from the group consisting of: 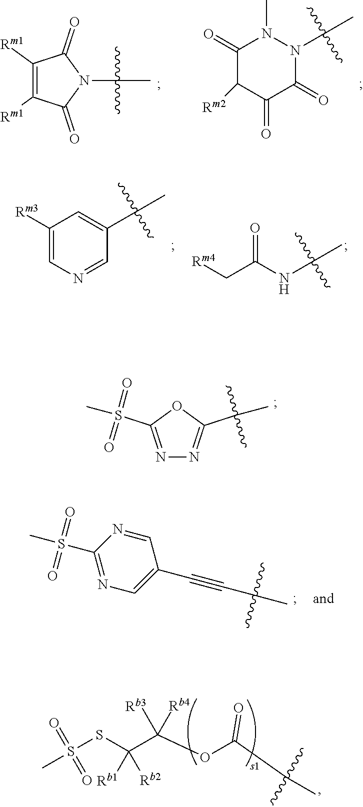
wherein
each R^{m1} and R^{m2} is independently hydrogen, halogen, or —S-Ph;
R^{m3} and R^{m4} are each halogen;
R^{b1} and R^{b2} are each independently hydrogen or C₁-C₆ alkyl, or R^{b1} and R^{b2} together with the carbon to which they are attached form a C₄-C₆ cycloalkyl or a C₄-C₆ heterocycloalkyl;
R^{b3} and R^{b4} are each independently hydrogen or C₁-C₆ alkyl,
subscript s1 is 0 or 1;
wherein the wavy line of M′ indicates the point of covalent attachment to the remainder of the structure of L′ and the wavy line of Formula (iii) indicates the point of covalent attachment to the degrader compound (D); and
U is absent or is —(CH₂)_{b}(R^{v1})ₛₛ(C═O)ᵤ(NH)ᵥ— wherein
subscript b is 0, 1, 2, 3, 4, or 5;
subscript ss is 0 or 1;
subscript u is 0 or 1;
subscript v is 0 or 1;
R^{v1} is —C₃-C₆ cycloalkyl- or —C₃-C₆ cycloalkyl-C(O)NHCH₂—R^{v2}—, wherein R^{v2} is C₃-C₆ cycloalkyl, C₃-C₆ heterocycle, or C₃-C₆ heteroaryl; or
—(CH₂CH₂O)_{w}CH₂CH₂(NH)—, wherein subscript w is an integer ranging from 1 to 16;
X is absent or is
—(CH₂CH₂O)_{w}CH₂CH₂—(C═O)_{d}—, wherein subscript w is an integer ranging from 0 to 16 and subscript d is 0 or 1;
—CH(R^{x1})(CH₂)ₙₙC(O)—, wherein R^{x1} is hydrogen, —COOH, —C(O)NHCH₃, or —(C(O)NHCH₂)ₓ(CH₂OCH₂)_{y}R^{x2}, wherein R^{x2} is —CH₂NHC(O)CH₃, —CH₂NH₂, or —CH₂C(O)OR^{x2a}, wherein R^{x2a} is hydrogen or C₁-C₆ alkyl; subscript nn is an integer ranging from 1 to 6; subscript x and subscript y are each independently an integer ranging from 0 to 8;
—C(O)—C(R^{x3})(R^{x4})—C(O)—, wherein R^{x3} and R^{x4} are independently hydrogen or C₁-C₆ alkyl or R^{x3} and R^{x4} together with the carbon to which they are attached form a C₃-C₆ cycloalkyl;
-heterocycloalkyl-O(CH₂)ᵢC(O)—, wherein subscript i is an integer ranging from 0 to 6;
—C(O)—C(R^{x5})(R^{x6})—, wherein R^{x5} is hydrogen or C₁-C₆ alkyl and R^{x6} is hydrogen, C₁-C₆ alkyl, or 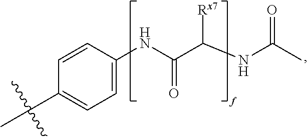
wherein subscript f is an integer ranging from 0 to 4 and each R^{x7} is independently hydrogen, —COOH, —NH₂, C₁-C₆ alkyl, C₁-C₆ alkyl, or an independently selected side chain of an amino acid; or
—(CH₂CH₂O)_{yy}CH₂CH₂NHC(O)CH₂OCH₂C(O)—, wherein subscript yy is an integer ranging from 0 to 16;
YY is a branching unit having the structure of: 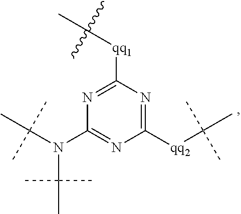
wherein
qq₁ and qq₂ are each independently —N(R^{y1})— or —O—, wherein R^{y1} is hydrogen or C₁-C₆ alkyl, and
the wavy line of YY indicates the point of attachment to X, when present, or to U when X is absent, or to M when X and U are absent; and
the dashed lines of YY indicate the point of attachment to EE when EE is present, or to AA when EE is absent, or to J when AA and EE are absent, or to G when AA, EE, and J are absent, and to NN;
NN is —(CH₂CH₂O)_{k′}CH₂CH₂C(O)—Rⁿⁿ¹—, wherein subscript k′ is an integer ranging from 0 to 16 and Rⁿⁿ¹ is hydrogen, C₁-C₆ alkyl, or —OH;
each EE is independently absent or —(CH₂CH₂O)_{x′}CH₂CH₂C(O)—, wherein subscript x′ is an integer ranging from 0 to 16;
each AA is independently absent or has the structure of: 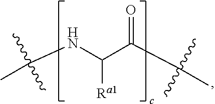
wherein subscript c is an integer ranging from 1 to 12; each R^{a1} is independently —COOH, —NH₂, or an independently selected side chain of an amino acid;
each J is independently absent, —(R^{j3})N(C(R^{j1})(R^{j2}))ₘ₁—, or has the structure of: 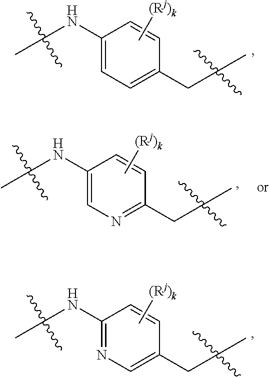
wherein
R^{j3} is hydrogen, C₁-C₆ alkyl, or C₃-C₆ cycloalkyl;
each R^{j1} and R^{j2} is independently hydrogen, C₁-C₆ alkyl, C₃-C₆ cycloalkyl, —OH, or —NR^{j4}R^{j5}, wherein R^{j4} and R^{j5} are each —OH or C₁-C₆ alkyl;
subscript m1 is an integer ranging from 1 to 6;
each R^{j} is independently halogen, C₁-C₆ alkyl, or —C(O)NH(C₁-C₆ alkyl);
subscript k is an integer ranging from 0 to 4;
each G is independently absent, 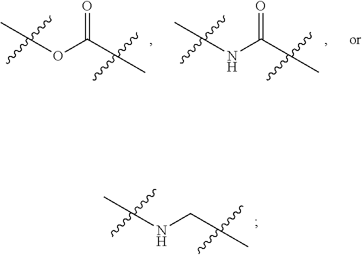
and
wherein the wavy line to each G of Formula (iib) indicates the point of covalent attachment to an independently selected D.

133. An Antibody Drug Conjugate compound of Table 1, or a pharmaceutically acceptable salt thereof.

134. A Degrader-Linker compound of Table 2, or a pharmaceutically acceptable salt thereof.

135. The Antibody Drug Conjugate compound of any one of claims 1 to 67, or a pharmaceutically acceptable salt thereof, wherein Ab binds to CALR antigen.

136. The Antibody Drug Conjugate compound of claim 135, or a pharmaceutically acceptable salt thereof, wherein Ab is an anti-CALR antibody.

137. A method of preparing the Antibody Drug conjugate compound of claim 135 or 136, the method comprising:
buffer exchanging a solution of a reduced Ab to produce a buffer exchanged solution of the reduced Ab; and
contacting a degrader-linker compound with the buffer exchanged solution of the reduced Ab to produce the Antibody Drug Conjugate compound.
